(19) United States
(12) Patent Application Publication
(10) Pub. No.: US 2026/0209374 A1
(43) Pub. Date: Jul. 23, 2026

(54) TNFR1 ANTAGONISTS LACKING AGONIST ACTIVITY AND USES THEREOF

(71) Applicant: ENOSI THERAPEUTICS CORPORATION, Eugene, OR (US)

(72) Inventors: H. Michael SHEPARD, Eugene, OR (US); Pei JIN, Palo Alto, CA (US)

(21) Appl. No.: 19/554,453

(22) Filed: Mar. 2, 2026

Related U.S. Application Data
(63) Continuation of application No. PCT/US24/44540, filed on Aug. 29, 2024.
(60) Provisional application No. 63/579,491, filed on Aug. 29, 2023.

(51) Int. Cl. C07K 16/28; A61P 11/00; A61P 19/02; A61P 37/06; C07K 14/765

(52) U.S. Cl. CPC C07K 16/2878; A61P 11/00; A61P 19/02; A61P 37/06; C07K 14/765; C07K 2317/569; C07K 2319/30

(57) ABSTRACT

Provided are molecular constructs that target tumor necrosis factor receptor 1 (TNFR1). The constructs are for treating diseases, disorders, and conditions in which TNFR1 and/or TNF is/are involved in the etiology or in which their inhibition or activation thereof can ameliorate the disease, disorder, and condition or a symptom thereof. Among the constructs provided herein, are TNFR1 antagonist constructs that are engineered to inhibit TNFR1 function; the constructs do not exhibit TNFR1 agonist activity. Methods of treatment of the various diseases in which TNF and its receptors play a role also are provided.

### RELATED APPLICATIONS

This application is a continuation of International PCT application No. PCT/US2024/044540, filed, Aug. 29, 2024, entitled “TNFR1 ANTAGONISTS LACKING AGONIST ACTIVITY AND USES THEREOF,” to inventors H. Michael Shepard and Pei Jin, and to Applicant Enosi Therapeutics Corporation, which claims benefit of priority to U.S. provisional application Ser. No. 63/579,491, filed Aug. 29, 2023, entitled “TNFR1 ANTAGONISTS LACKING AGONIST ACTIVITY AND USES THEREOF,” to inventors H. Michael Shepard and Pei Jin, and to Applicant Enosi Therapeutics Corporation.

Benefit of priority also is claimed to U.S. provisional application Ser. No. 63/579,491, filed Aug. 29, 2023, entitled “TNFR1 ANTAGONISTS LACKING AGONIST ACTIVITY AND USES THEREOF,” to inventors H. Michael Shepard and Pei Jin, and to Applicant Enosi Therapeutics Corporation.

This application is related to International PCT application No. PCT/US2021/048074, filed Aug. 27, 2021, published as International PCT application No WO 2022/047243 on Mar. 3, 2022, and U.S. application Ser. No. 17/731,595, filed on Apr. 28, 2022, and published as US Publication No.: 2022/0288226 on Sep. 15, 2022, each to inventor H. Michael Shepard, and Applicant Enosi Therapeutics Corporation, and each entitled “METHODS AND COMPOSITIONS TO TREAT AUTOIMMUNE DISEASES AND CANCER,” which claims the benefit of priority to U.S. Provisional Application Ser. No. 63/071,313, filed Aug. 27, 2020, entitled “METHODS AND COMPOSITIONS TO TREAT AUTOIMMUNE DISEASES AND CANCER” to inventor H. Michael Shepard, and Applicant Enosi Life Sciences Corp., and now assigned to Enosi Therapeutics Corporation.

This application also is related to International PCT application No. PCT/US2020/018739, filed Feb. 19, 2020, entitled “ANTIBODIES AND ENONOMERS,” published on Aug. 27, 2020, as International PCT Publication No. WO 2020/172218, to inventor H. Michael Shepard, and Applicant Enosi Life Sciences Corp., and now assigned to Enosi Therapeutics Corporation. This application also is related to the U.S. application Ser. No. 17/432,720, filed Aug. 20, 2021, which is the U.S. National Stage Application of PCT/US2020/018739, filed Feb. 19, 2020, which claims the benefit of priority to U.S. Provisional Application Ser. No. 62/808,635, filed Feb. 21, 2019.

The subject matter of each of these applications is incorporated by reference in its entirety.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file was created on Mar. 2, 2026, is 2,609,388 bytes in size, and is titled 5306SEQ001.xml.

### FIELD

This application is directed to nucleic acid constructs and encoded products for use as anti-TNF therapies. The treated diseases are those in which TNF receptors and/or TNF or the TNF/TNF receptor(s) pathways is involved or plays a role in the etiology thereof.

### BACKGROUND

Anti-TNF therapies/TNF-blockers (a type of biological Disease Modifying Anti-Rheumatic Drugs; DMARDs) typically are prescribed after the failure of conventional DMARDs. These therapies include monoclonal antibodies (mAbs), such as the chimeric mAb infliximab (Remicade®); containing a murine variable region and a human IgG1 constant region, and the fully humanized mAbs (IgG1s) adalimumab (sold, for example under the trademark Humira®), and golimumab (Simponi® antibody); the PEGylated humanized Fab′ fragment of a mAb targeting TNF, certolizumab pegol (Cimzia® antibody); and TNFR2 fusion proteins, such as the TNFR2-Fc fusion protein etanercept (sold under the trademark Enbrel®), which contains the extracellular receptor region that contains the binding site of human TNFR2 fused to the Fc of human IgG1. The drugs sold under the trademarks Remsima® and Inflectra® are biosimilars of infliximab that are approved for use in the European Union for the treatment of various autoimmune and chronic inflammatory diseases and disorders. These TNF inhibitors, which sequester TNF, are used for the treatment of various diseases and conditions, including, for example, RA, psoriasis, psoriatic arthritis, ankylosing spondylitis, juvenile idiopathic arthritis (JIA), and/or inflammatory bowel disease (IBD; such as, Crohn's disease and ulcerative colitis).

Such therapies, however, are associated with severe side effects, including, for example, an increased risk of sepsis and serious infections, such as listeriosis, reactivation of tuberculosis, reactivation of hepatitis B/C, reactivation of herpes zoster, and invasive fungal and other opportunistic infections, including reactivation of M. tuberculosis infection. These therapies have been shown to induce macrophage apoptosis in the rheumatoid synovium. Infliximab is associated with increased apoptosis in the inflammatory cell infiltrate in the guts of patients with Crohn's disease. Other anti-rheumatic drugs, such as methotrexate and glucocorticoids, also can induce apoptosis in immune cells (see, e.g., Vigna-Perez et al. (2005) Clin. Exp. Immunol. 141(2):372-380). These therapeutic agents also can cause worsening of severe congestive heart failure, drug-induced lupus, and demyelinating central nervous system (CNS) diseases, as well as lymphomas and non-melanoma skin cancers (see, e.g., Benjamin et al. Disease Modifying Anti-Rheumatic Drugs (DMARDs) [Updated 2020 Feb. 27]. In: StatPearls [Internet]. Treasure Island (FL): StatPearls Publishing; 2020 January Available from: (ncbi.nlm.nih.gov/books/NBK507863/)). Other adverse side effects include liver injury, demyelinating disease/CNS disorders, lupus, psoriasis, sarcoidosis, and an increased susceptibility to the development of additional autoimmune diseases, as well as cancers, including lymphomas and solid malignancies (see, e.g., Dong et al. (2016) Proc. Natl. Acad. Sci. U.S.A. 113(43):12304-12309; Zalevsky et al. (2007) J. Immunol. 179:1872-1883; Zoran et al. (2019) Sci. Rep. 9:17231).

Thus, the uses of these therapeutic agents, particularly for chronic diseases/conditions that require long-term administration, such as arthritis and inflammatory bowel disease (IBD, are limited. Approximately 30% of RA patients are non-responsive, or therapeutic benefits are not sustained, with the use of anti-TNF therapies (see, e.g., McCann et al. (2014) Arthritis & Rheumatology 66(10):2728-2738). Non-responsiveness also occurs in non-RA patients receiving anti-TNF therapeutics. Depending on the anti-TNF agent, 13-33% of treated patients do not respond to treatment, and up to 46% stop responding, resulting in discontinuation or dose increase (see, e.g., Richter et al. (2019) MABS 11(4):653-665). Thus, there is a need for therapies with improved therapeutic efficacy and safety.

### SUMMARY

Provided are molecular constructs, and nucleic acids encoding them, that antagonize tumor necrosis factor receptor 1 (TNFR1). Constructs provided herein include those that do not have or have reduced TNFR1 agonist activity, or they do not have or have reduced TNFR2 antagonist activity compared to available TNFR1 antagonists. The constructs are for treating diseases, disorders, and conditions in which these receptors and/or TNF are involved in the etiology or in which their inhibition or activation can ameliorate the disease, disorder, and/or condition or a symptom thereof.

As discussed herein, bivalent antibodies that target TNFR1 reportedly are partially agonistic or fully agonistic by virtue of receptor clustering by the antibody (see, e.g., Richter et al., PMID 23977237, Richter et al., PMID 30252601, Belka et al., PMID 7720706, Mandik-Nayak et al., doi.org/10.4049/jimmunol.167.4.1920, Yu et al., doi.org/10.1038/s42003-021-02309-5). Prior art that describes TNFR1 antagonist antibody constructs showed that bivalency in anti-TNFR1 antibodies results in TNFR1 activation due to receptor clustering by the construct antibody. For example, US Patent Publication 2014/0112929 describes the agonistic activity of bivalent TNFR1 binding antibodies and states that such antibodies are not useful as inhibitors of TNFR1 because of the concomitant agonist activity. In contrast, it is shown herein, that among anti-TNFR1 bivalent constructs, such as constructs that contain dAbs or VhH domains, are those that, when provided as a bivalent construct that includes modified Fc's as described herein, do not have TNFR1 agonist activity. For example, the constructs designated herein as EN-1206 bivalent and monovalent that contain the dAb from SEQ ID NO:59, do not have agonist activity. In contrast, constructs that include the designation EN208 or EN1208, containing the dAb of SEQ ID NO:54 (DOM1 h-574-208) bivalent does have agonist activity. The dAbs in each of these constructs, 1206 vs 1208, bind different epitopes.

Constructs provided herein are those that bind to the same epitope as the dAb (SEQ ID NO:59) in the EN-1206 constructs and have no agonist activity in bivalent form or reduced agonist activity compared to the EN1208 bivalent construct. These include constructs that comprise one of the dAbs set forth in any of SEQ ID NOs: 55-84, 86-88, 450, 495-498, which bind to the same epitope as the EN1206 constructs, which constructs contain the dAb (SEQ ID NO:59) and variants thereof that have at least 95% sequence identity to one or more of the dAbs set forth in any of SEQ ID NOs: 55-84, 86-88, 450, 495-498 and retain binding to the same epitope as the EN1206 constructs.

Identification of dAbs with such properties are detailed herein; see FIG. 19 and accompanying text. dAbs that bind to the same epitope as EN1206 include those that comprises CDRS that have at least 95% sequence identity to HCDR1, HCDR2, and HCDR3 of DOM1 h-131-511 (SEQ ID NO: 60), which binds to domain 3 of TNFR1 extracellular domain. FIG. 18 shows the CDRs for DOM1 h-131-511 and DOM1 h-131-206 derived therefrom (SEQ ID NO:59). EN1206 constructs contain the dAb DOM1 h-131-206.

The constructs contained herein are monovalent or divalent constructs that contain one or two, respectively, dAbs linked directly or via a polypeptide linker to an Fc, generally a modified Fc. Modified Fc's are detailed below. Among the dAbs are those of SEQ ID NOs: 54-672 that when in the bivalent construct have antagonist activity and do not have agonist activity. In particular, the dAb comprise one of the dAbs set forth in any of SEQ ID NOs: 55-84, 86-88, 450, 495-498 and variants there that have at least 95% sequence identity thereto and bind to the same epitope as EN 1206. EN1206 (DOM1 h-131-206; SEQ ID NO: 59) binds to the domain 3 of TNFR1 ECD with an affinity of 407 pM measured by SPR. The DOM1 h-131-206 was optimized for affinity and stability based on DOM1 h-131-511. As detailed herein, EN-1000-206 (‘206’) is a VhH single domain polypeptide, EN-1206-Fc-MV is a knobs in hole construct pairing an IgG1 Fc-only domain to a fusion of 206-IgG1-Fc).EN-1206-Fc-BV is a homodimeric/bivalent 206-IgG1-Fc; and EN-1208-Fc-BV is Same as EN-1206-Fc-BV except the VhH domain was EN-1000-208 (containing the dAb of SEQ ID NO:54), a different VhH domain fused with IgG1-Fc to form a bivalent antibody-like protein. 1208 is binds to a different epitope on TNFR1 from the dAb in the 1206s (see, FIGS. 8A and 8B, which depict the structures).

The constructs also can include all or a portion of nanobodies (SEQ ID NOs: 480-487) that bind to TNFR1, and that, when in the constructs, result in constructs that are TNFR1 antagonists that do not antagonize TNFR2 and that are not TNFR1agonists. Linkers and modified Fc's for including in the constructs are described throughout the disclosure. The constructs can be monovalent, but in general are bivalent. When monovalent, the Fc's can include holes and knobs.

Suitable dAbs can be identified by the methods exemplified in the Examples. Selection constructs that do not have agonist activity can be effected by screening, such as identifying those with high affinity binding specific for TNFR1, such as in the nM range of affinity; followed by screening using the THP1 assay (see the Examples) for inhibition of TNF-stimulated expression of nuclear factor kappa-light-chain-enhancer of activated B cells (NF-κB) and or expression of inflammatory cytokines, and selecting the constructs that lack agonistic activity in the THP1 assay. Alternatively, the constructs contain dAbs that bind to the same epitope as the dAb of SEQ ID NO:59 (Dom-131-206). Hence also provided are methods for identifying TNFR1 antagonists that do not have agonist activity. For discussions regarding the role of cytokines and the NF-κB signaling in immunity see, e.g., Yoshimura et al., PMID 28716890; Front Immunol. 2021; 12: 705485, Published online 2021 Jul. 7. doi: 10.3389/fimmu.2021.705485; see, also doi: 10.1080/19420862.2019.1596512; ncbi.nlm.nih.gov>articles>PMC3011218 by K A Zettlitz (2010), which describes In vitro, ATROSAB inhibited TNF-mediated responses, such as apoptosis induction and activation of NFκB-dependent gene expression, such as IL-6 and IL-8; ncbi.nlm.nih.gov>articles>PMC8294390 by F Richter.

As shown in the application the 1206 and 1208 constructs (detailed in the Examples) meet the criteria of specific binding to TNFR1, and of blocking TNF binding to TNFR1, but they differ in activity as demonstrated in assays that show showing agonism. These assays can be used and were used to identify constructs that are TNFR1 antagonists and that do not have TNFR1 agonist activity. Such constructs are provided herein.

Cells have two TNF receptors: TNFR1 and TNFR2. These pathways balance one another in normal physiology. TNF/TNFR1 drives inflammation, while TNF/TNFR2 is anti-inflammatory. TNFR2 generally is activated later than TNFR1, and so does not immediately impact useful TNF-induced inflammation but activates later to suppress over activation of inflammatory pathways. Simultaneous inhibition of both pathways removes the inflammation-dampening effect of TNFR2. Existing TNF blockers limit their own efficacy because the Treg generator (TNFR2), which is anti-inflammatory, is turned down/off.

The constructs provided herein, among other properties that differ from prior therapeutics that target TNF/TNFRs, inhibit TNFR1 signaling or activity without compromising the ability of a treated subject to fight opportunistic infections. Among the constructs provided herein is one type that is a modified single chain antibody or other configuration as detailed herein that specifically targets and inhibits TNFR1, but does not antagonize TNFR2, and does not exhibit transient activation of TNFR1 via receptor clustering. Constructs provided herein silence the TNF inflammatory pathway mediated by TNFR1, but retain, and in some embodiments enhance, the healing pathway of TNFR2. These constructs can be administered to treat indications where TNF blockers have failed.

Among the constructs provided herein are constructs that specifically inhibit tumor necrosis factor receptor type 1; provided are methods and uses of the constructs for treating diseases, disorders, and conditions in which TNF or receptors therefor play a role in the etiology or in the symptoms.

Existing anti-TNF drugs block overzealous inflammation, which occurs in autoimmune diseases, including rheumatoid arthritis, polyarticular juvenile idiopathic arthritis, axial spondylarthritis, ankylosing spondylitis, psoriatic arthritis, psoriasis, Crohn's disease, pediatric Crohn's disease, and ulcerative colitis. The constructs herein can be used to treat the same diseases, but avoid the deleterious or adverse side effects. Constructs provided herein are more effective at suppressing inflammatory cytokines in vivo than prior therapeutics, such as the TNFR2-Fc fusion protein etanercept (sold under the trademark Enbrel®), and preserve regulatory T-cell function. The constructs can include activity modifiers or property modifiers to increase serum half-life, and have demonstrated activity in blocking TNFR1 signaling, such as in TNF assays that compare activity with adalimumab (brand name, e.g., Humira®) and/or etanercept (sold as Enbrel®).

The constructs preserve macrophage function better than adalimumab, showing they do not lead to opportunistic infections; they also preserve Treg function substantially better than adalimumab (e.g., sold as Humira®) or etanercept (sold under the trademark Enbrel®), and are as therapeutically effective in treating diseases, disorders, and conditions, such as rheumatoid arthritis. In some embodiments, the Kd is <1 nM, and the t_{1/2} in vivo is about 10-12 days. The constructs can be administered by any suitable route for the particular indication. Routes include, but are not limited to, subcutaneously, intravenously, intratumorally, intra-hepatically, topically, mucosally, intradermally, and any other suitable route.

Specific constructs provided herein are the monovalent and bivalent constructs that comprise the dAb of SEQ ID NO:59 linked to an Fc that is modified for increased half-life with the mutations YTE as detailed below. The monovalent construct (EN1206-MV; see FIG. 8A) comprises a first chain that is the modified Fc that also is modified to include a “knob,” a second chain with the dAb linked to the modified Fc that also is modified to include a “hole,”, to form the two chain structure. The Fc portions also include mutations to mutations to minimize its in vivo Fe effector functions. The sequences are as follows:

Chain 1 of EN1206 monovalent - SEQ ID NO:1505 containing modified Fc hole:
Fc only (hole: T366S, L368A, Y407V); half- life increase - YTE
(M252Y, S254T, T256E), and minimized its in vivo Fc effector function - LALAPG
(L234A, L235A, P329G)
DKTHTCPPCPAPEAAGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALGAPIE
KTISKAKGQPREPQVYTLPPSRDELTKNQVSLSCAVKGFYPSDIAVEWESNGQPENN
YKTTPPVLDSDGSFFLVSKLTVDKSRWQQGNVFSCSVMHEALHNHYTQKSLSLSPGK
Chain 2 of EN1206 monovalent dAB with Fc knob modified for increased half-life
and minimized Fc effector function - One-armed VHH-Fc (knob: T366W) including YTE
(M252Y, S254T, T256E) LALAPG (L234A, L235A, P329G))(SEQ ID NO: 1506)
EVQLLESGGGLVQPGGSLRLSCAASGFTFAHETMVWVRQAPGKGLEWVSHIPPDGQ
DPFYADSVKGRFTISRDNSKNTLYLQMNSLRAEDTAVYHCALLPKRGPWFDYWGQG
TLVTVSSDKTHTCPPCPAPEAAGGPSVFLFPPKPKDTLYITREPEVTCVVVDVSHEDPE
VKFNWYVDGVEVHNAKTKPREEQYNSTYRVVSVLTVLHQDWLNGKEYKCKVSNK
ALGAPIEKTISKAKGQPREPQVYTLPPSRDELTKNQVSLWCLVKGFYPSDIAVEWESN
GQPENNYKTTPPVLDSDGSFFLYSKLTVDKSRWQQGNVFSCSVMHEALHNHYTQKS
LSLSPGK
The bivalent construct, designated EN1206-BV, includes two identical chains.
Both chains of EN1206 bivalent include the dAb and Fc with modifications - YTE
(M252Y, S254T, T256E)LALAPG(L234A, L235A, P329G)(see, (SEQ ID NO: 1507):
EVQLLESGGGLVQPGGSLRLSCAASGFTFAHETMVWVRQAPGKGLEWVSHIPPDGQ
DPFYADSVKGRFTISRDNSKNTLYLQMNSLRAEDTAVYHCALLPKRGPWFDYWGQG
TLVTVSSDKTHTCPPCPAPEAAGGPSVFLFPPKPKDTLYITREPEVTCVVVDVSHEDPE
VKFNWYVDGVEVHNAKTKPREEQYNSTYRVVSVLTVLHQDWLNGKEYKCKVSNK
ALGAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKS
LSLSPGK

In contrast, the construct designated EN1208-BV YTE-LALAPG includes two chains containing the dAb (SEQ ID NO:54) linked to the modified Fc; YTE (M252Y,S254T T256E) LALAPG (L234A,L235A,P329G):

EVQLLESGGGLVQPGGSLRLSCAASGFTFDKYSMGWVRQAPGKGLEWVSQ
ISDTADRTYYAHAVKGRFTISRDNSKNTLYLQMNSLRAEDTAVYYCAIYT
GRWVPFEYWGQGTLVTVSSDKTHTCPPCPAPEAAGGPSVFLFPPKPKDTL
YITREPEVTCVVVDVSHEDPEVKFNWYVDGVEVHNAKTKPREEQYNSTYR
VVSVLTVLHQDWLNGKEYKCKVSNKALGAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK

As shown herein, each of EN1208-MV and BV has TNFR1 agonist activity: MGWSCIILFLVATATGVHS (SEQ ID NO:1509) is the leader sequence linked, upon expression, to the N-terminus of each chain, that is cleaved upon expression the cell. The Knob-into-hole mutations are engineered into the monovalent EN1206 for its heterodimer paring; the YTE mutations are engineered into the Fc to extend its in vivo half-life; the “LALAPG,” which refers to the replacements, by EU numbering: L234A/L235A/P329G, mutations are engineered into the Fc to minimize its in vivo Fc effector function; and numbering for the Fc modifications is EU numbering.

As demonstrated in the Examples, the EN1206 constructs do not exhibit TNRF1 agonist activity, and are TNRFR1 antagonists. As detailed herein, the EN1206 constructs comprise the dAb of SEQ ID NO:59, and the EN1208 constructs comprise the dAb of SEQ ID NO:54. The EN1206 constructs in all formats and forms do not have TNFR1 agonist activity; the EN1208 constructs do. Thus, constructs provided herein are EN1206 constructs or those that have TNFR1 inhibitor portions such that the resulting construct does not have TNFR1 agonist activity.

Other constructs that include a dAb or dAbs are those that bind to the same epitope as the dAb of SEQ ID NO:59 or SEQ ID NO: 60 also have antagonist activity, and not have agonist activity. In contrast, it is shown herein, that the EN1208 constructs have agonist activity. The dAb in the 1208 construct binds to a different epitope on TNFR1. Constructs that contain dAb's that bind to the same epitope as the dAb in the 1206 constructs. EN1206 (DOM1 h-131-206; SEQ ID NO: 59) binds to the domain 3 of TNFR1 ECD with an affinity of 407 pM measured by SPR. The dAb DOM1 h-131-206 (SEQ ID NO:59) was optimized for affinity and stability based on DOM1 h-131-511 (SEQ ID NO:60). The dAb are identified, and include the dAbs set forth in any of SEQ ID NOs: 55-84, 86-88, 450, 495-498 bind to the same epitope as EN 1206. Also included are dAbs and constructs containing the dAbs that comprise CDRs that have at least 95% sequence identity to HCDR1, HCDR2, and HCDR3 of DOM1 h-131-511 (SEQ ID NO: 60), which binds domain 3 of TNFR1 extracellular domain. FIG. 18 shows the CDRs for 511 and 206.

Constructs include those that comprise the dAb of SEQ ID NO:59 or a variant thereof having at least 95% sequence identity thereto and binding to the same epitope as SEQ ID NO:59 linked directly or indirectly via a linker to a modified Fc, wherein the Fc is modified to have increased half-life. These include the constructs designated EN1206-BV or EN1206-MV, wherein the EN1206-MV construct comprises Fc's with knobs into holes. Also provided are constructs that comprise the sequence of amino acids set forth in any or combinations of SEQ ID NOs: 1488-1490 and/or of SEQ ID NOs: 1505-1507 and variants thereof that bind TNRF1 and have anti-TNFR1 antagonist activity and retain the half-life of constructs that comprise SEQ ID NO:1488-1491.

Provided are monovalent constructs that comprise SEQ ID NO: 1489, the dAb-knob chain, linked to SEQ ID NO:1490, and the Fc hole chain, and variants thereof that bind TNRF1 and have anti-TNFR1 antagonist activity and retain the half-life of constructs, where the knob and hole Fc portions form an Fc multimer that displays the dAb portion.

Also provided are bivalent constructs that comprise the chain of SEQ ID NO: 1492 and variants thereof that bind TNRF1 and have anti-TNFR1 antagonist activity and retain the half-life of constructs.

Provided are constructs that are a tumor necrosis factor receptor 1 (TNFR1) antagonist construct of formula 1: (TNFR1 inhibitor)ₙ-linkerₚ-(activity modifier)_{q}, wherein: each of n and q is an integer, and each is independently 1, 2, or 3; p is 0, 1, 2 or 3; a TNFR1 inhibitor is a molecule that binds TNFR1 to inhibit (antagonize) activity of TNFR1; an activity modifier is a moiety that modulates or alters the activity or a pharmacological property of the construct compared to the construct in the absence of the activity modifier; and linkers increases flexibility of the construct, and/or moderates or reduces steric effects of the construct or its interaction with a receptor, and/or increases solubility in aqueous media of the construct. Linkers can contain a plurality of components. Linkers include chemical linkers, polypeptide linkers, and combinations thereof. The constructs can be linked via chemical and/or physical bonds. The constructs can be fusion proteins.

The TNFR1 inhibitor can comprise a domain antibody (dAb) or a single chain antibody. The construct includes those in which the TNFR1 inhibitor is a domain antibody (dAb), the activity modifier is not an unmodified single Fc region or a human serum albumin antibody. The constructs generally are bivalent and are those that are selected so that they do not have agonist activity. For example, the activity modifier (or property modifier) is a modified Fc region or is human serum albumin. In the constructs, the TNFR1 inhibitor can be one that inhibits TNFR1 signaling, and/or the activity modifier increases serum half-life of the construct. For example, the constructs include those in which the activity modifier is albumin or an Fc that is modified to have reduced or no ADCC (antibody dependent cellular cytotoxicity) activity and/or reduced or no CDC (complement-dependent cytotoxicity) activity. The TNFR1 inhibitor can be one that inhibits a TNFR1 activity, but does not antagonize tumor necrosis factor receptor 2 (TNFR2) activity. The TNFR1 inhibitor can be one that inhibits TNFR1 signaling.

Also provided are multi-specific constructs. For example, provided are multi-specific constructs, comprising a TNFR1 inhibitor and a Treg expander, wherein a bi-specific construct interacts with two different target receptors or antigens or epitopes on a receptor. Among the multi-specific constructs are those that are bi-specific for TNFR1 and a Treg expander. The Treg expander can be a TNFR2 agonist.

The constructs can comprise a linker to provide flexibility, increase solubility, and/or to relieve and/or reduce steric hindrance and/or Van der Waals interactions. The constructs, optionally, but generally comprise an activity modifier to alter or modulate the activity or a property of the construct. Provided are constructs that have Formula 2: (TNFR1 inhibitor)₁-(activity modifier)ᵣ₁ (Linker (L))ₚ-(activity modifier)ᵣ₂ (TNFR2 agonist)_{q}, or (TNFR1 inhibitor)₁-(activity modifier)ᵣ₁-(Linker (L))ₚ-(activity modifier)ᵣ₂ (Treg expander)_{q}, where: n=1, 2, or 3, p=1, 2, or 3, q=0, 1 or 2, and each of r1 and r2 is independently 0, 1, or 2; and the components can be in the order specified or any other order as long as the construct interacts with TNFR1 and TNFR2 to antagonize TNFR1 and agonize TNFR2, or has Treg expander activity. For example, included are constructs, among any of those provided herein, where the TNFR1 inhibitor moiety inhibits binding of TNF-α binding to TNFR1 and/or inhibits signaling.

Also provided are constructs of formula 3a or 3b: (TNFR2 agonist or Treg expander)₁-linkerₚ-(activity modifier)_{q}, formula 3a, or (activity modifier)_{q}-linkerₚ-(TNFR2 agonist or Treg expander)ₙ, formula 3b, where: each of n and q is an integer, and each is independently 1, 2, or 3; p is 0, 1, 2 or 3; an activity modifier is a moiety that alters a pharmacological property or an activity of the construct; a TNFR2 agonist interacts with TNFR2 resulting in TNFR2 activity; a Treg expander, includes TNFR2 agonists, and is a molecule that results in increased Treg cells; and a linker increases flexibility and/or moderates or reduces steric effects of the construct or its interaction with a receptor; and/or alters solubility of the construct. In some embodiments, the activity modifier is an Fc region or a modified Fc region or a short FcRnBP; and the linker comprise a hinge region, or is a linker comprising G and S residues. Exemplary of linkers are those that increase serum half-life of the construct. For example, the linker can have a sequence set forth in any of SEQ ID NOs: 812-834 or is a PEG moiety linker. In some embodiments, the construct comprises an activity modifier that is a modified Fc region or a peptide that increases serum half-life of the construct. The Fc region can be an Fc dimer; the Fc region can be modified to have reduced ADCC and/or CDC activity, such as an Fc modified to have reduced or no ADCC activity.

Included among the constructs provided herein are those in which the TNFR1 inhibitor is any as defined in the sequence listing, listed below, or known in the art; the Treg expander is any known in the art, a TNFR2 agonist, or any Treg expander set forth in the sequence listing, or known in the art; the linker is any listed in the sequence listing or below or known in the art; and the activity modifier is any set forth in the sequence listing, known in the art, and/or set forth below.

Provided are constructs that are TNFR1 antagonist constructs, comprising a TNFR1 inhibitor that is a single chain antibody or antigen-binding portion thereof that specifically targets and inhibits TNFR1, but does not antagonize TNFR2, thereby preventing transient activation of TNFR1 via receptor clustering. In such constructs that comprise an antibody or antigen-binding portion thereof, the antibody or antigen-binding fragment thereof can contain a modification that improves a pharmacological property and/or structure of the construct.

In any of the constructs provided herein, the constructs include component(s) that agonize(s) TNFR2 signaling to thereby increase expression of regulatory T cells (Tregs), thereby providing TNFR1 antagonism and concomitant (or substantially concomitant) increase in expression of Tregs. In the constructs provided herein, the TNFR1 inhibitor can be a single chain antibody that inhibits TNFR1 by inhibiting TNFR1 signaling, such as, for example, where the antibody portion or antigen binding portion of the construct inhibits binding of TNF-α binding to TNFR1. Among the constructs are those where the TNFR1 inhibitor is an antibody or antigen binding portion that does not inhibit binding of TNF-α to TNFR1, but does inhibit TNFR1 signaling. The property or activity that can be modulated/altered can be serum half-life.

The constructs can comprise an Fc modified to eliminate ADCC and/or CDC activity. The construct can comprise an Fc dimer, such as one in which one Fc monomer comprises holes, and the other comprises knobs, to form a heterodimer. For example, the knob mutation(s) is/are selected from among S354C, T366Y, T366W, and T394W by EU numbering; and the hole mutation(s) is/are selected from among Y349C, T366S, L368A, F405A, Y407T, Y407A, and Y407V by EU numbering, whereby the Fc monomers form the heterodimer. In some embodiments where the construct comprises an Fc, the Fc is from trastuzumab. The construct can be dimerized by fusion of the N-terminus with the C-terminus of trastuzumab.

In some embodiments in which the constructs comprise a linker the linker is or comprises a hinge region from an Fc region. For example, in which the hinge region is from trastuzumab, and it is linked to the Fc region. The constructs include those that comprise a linker that is linked to the anti-TNFR1 antagonist antibody or antigen-binding portion thereof. The linker can be linked to the anti-TNFR1 antagonist antibody or antigen-binding portion thereof, and directly or via a hinge region to an Fc region. The Fc region or modified Fc region, for example, comprises the sequence of amino acids set forth in any of SEQ ID NOs:10, 12, 14, 16, 27, 30, 1469, and 1470.

Also provided are constructs that bind to neonatal Fc receptor (FcRn). For example, provided are TNFR1 constructs that comprise a short FcRn-binding peptide (FcRnBP), where a short FcRn-binding peptide (FcRnBPs) provides for the interaction of the construct with FcRn, and contains 6-25, or 10-20 amino acid residues. For example, the FcRnBP contains 12-20 residues or 15 residues or 16 residues. Exemplary of these are TNFR1 antagonist constructs where the FcRn-binding peptide (FcRnBP) comprises or consists of a peptide of any SEQ ID NOs:48-51. The constructs include TNFR1 constructs that comprise an Fc heterodimer, where one Fc monomer comprises holes, and the other comprises knobs, whereby the Fc dimer that results is a heterodimer.

Provided are constructs that are TNFR1 antagonist constructs that comprise: a TNFR1 inhibitor; an Fc dimer; and a Treg expander, where: the Fc dimer comprises two complementary Fc monomers; the TNFR1 inhibitor is linked to one of the Fc monomer, and the Treg expander is linked to the other Fc monomer. In such constructs the Treg expander can be a TNFR2 agonist. They can further comprise a second Treg expander linked to the same Fc monomer as the TNFR1 inhibitor, where the first and second Treg expanders are the same or different. The second Treg expander can be a TNFR2 agonist. In some embodiments, the Treg expanders are the same. The TNFR1 inhibitor can be one that inhibits or blocks TNFR1 signaling. In some embodiments, the TNFR1 inhibitor binds to TNFR1 and blocks or inhibits TNF-a binding and TNFR1 signaling. In some embodiments, the TNFR1 inhibitor binds to TNFR1, does not or interfere with TNF-α binding, and blocks or inhibits TNFR1 signaling. In some embodiments of these constructs, the Treg expander is a TNFR2 agonist. The TNFR2 agonist can be one that stimulates or induces TNFR2 signaling. Exemplary of the Treg expanders is a TNFR2 agonist that is an scFv, VHH single domain antibody, or Fab of aTNFR2 agonist monoclonal antibody. In these constructs, the Treg expander can be a TNFR2 agonist that is a small molecule, or a nucleic acid aptamer, or a peptide aptamer.

Also provided are any of these constructs that is or also is a TNFR2 agonist. The TNFR2 agonist is a construct of formula 3a or 3b, where: formula 3a is (Treg expander)₁-linkerₚ-(activity modifier)_{q}, and formula 3b is (activity modifier)_{q}-linkerₚ-(Treg expander)ₙ. In these formulae, each of n and q is an integer, and each is independently 1, 2, or 3; p is 0, 1, 2 or 3; an activity modifier is a moiety that modulates or alters the activity or a pharmacological property of the construct compared to the construct in the absence of the activity modifier; and the linker increases flexibility of the construct, and/or moderates or reduces steric effects of the construct or its interaction with a receptor, and/or increases solubility in aqueous media of the construct. In any of these constructs, the Treg expander in the construct is a TNFR2 agonist. For example, the TNFR2 agonist stimulates or induces TNFR2 signaling. In other examples, the Treg expander is a TNFR2 agonist that is an scFv, VHH single domain antibody, or Fab of a TNFR2 agonist monoclonal antibody. The Treg expander can be a TNFR2 agonist that is a small molecule, or a nucleic acid, or peptide aptamer. In the constructs that comprise all or a portion of trastuzumab, such as the Fc portion and/or Fc and hinge region or modified forms thereof, the construct can be dimerized by N-terminal fusion with the C-terminus of trastuzumab.

Provided are constructs that comprise a TNFR1 inhibitor moiety linked via a central PEG linker to one more Treg expanders, or that comprise at least two TNFR1 inhibitors that are the same or different, or that comprise two Treg expanders that are the same or different. The constructs that comprise a PEG moiety, such as a central PEG linker can comprise a branched PEG moiety linking the TNFR1 inhibitor and one or more Treg expanders. Exemplary are those that have a structure selected from among formulae 4A to 4D:

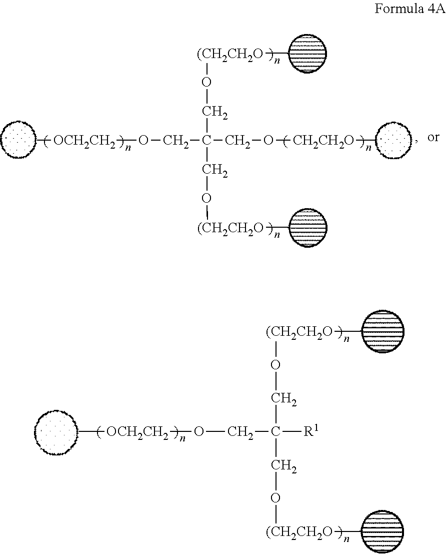

n is 1 to 5;
R¹ is H or CH₃, or CH₂CH₃ or other C1-C5 alkyl
[symbol] is aTNFR1 inhibitor (TNFR1 antagonist);
[symbol] is a Treg expander; or

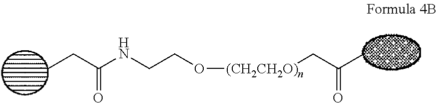

[symbol] is a TNFR1 inhibitor (TNFR1 antagonist)
[symbol] is a Treg expander;
n is 1 to 5; or

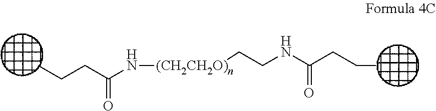

[symbol] is a TNFR1 inhibitor (TNFR1 antagonist), or a Treg expander; and
n is 1 to 5; or

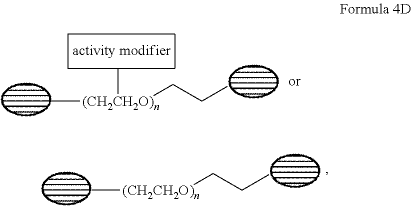

wherein
each [symbol] is same or different and each is independently selected from a TNFR1 inhibitor (TNFR1 antagonist), and a TNFR2 agonist;
the activity modifier is optional, and can be linked to any suitable locus in the molecule; and n is 1 to 5.

In TNFR1 antagonist constructs and other constructs provided herein, the Treg expander can be a TNFR2 agonist. These constructs can include an activity modifier, such as, for example, where the activity modifier is an Fc region, or is an Fc region that includes a hinge region or other linker; and the Fc region or Fc region with hinge region is an Fc that is modified to reduce or eliminate ADCC and/or CDC activity. Exemplary thereof are constructs where the Fc or modified Fc is an IgG Fc or is an IgG1 or IgG4 Fc, and/or are constructs that bind to neonatal Fc receptor (FcRn). Exemplary of these constructs are those where: the construct comprises a short FcRn-binding peptide (FcRnBP), where the short FcRn-binding peptide (FcRnBPs) provides for the interaction of the construct with FcRn, and contains 6-25, such as 10-20 amino acid residues; or wherein the FcRnBP contains 12-20 residues or 15 residues or 16 residues, such as, for example where the FcRn-binding peptide (FcRnBP) comprises or consists of a peptide of any SEQ ID NOs:48-51.

Also provided are TNFR1 antagonist constructs of any of the formulae above and in the application that comprise: a) a TNFR1 inhibitor moiety that is a TNFR1-selective; b) optionally, one or more linkers; and c) optionally, a half-life extending moiety, where the antagonist construct comprises at least one of b) and c). In such constructs, the TNFR1-selective antagonist selectively binds and inhibits TNFR1 signaling, but not TNFR2 signaling. Additionally, as described herein, there resulting construct is one that does not have TNFR1 agonist activity. As exemplified, for example, in the Examples, the constructs designated 1206, such as the monovalent construct and the divalent construct, that contain the dAb from SEQ ID NO:59, linked via a polypeptide linker, to a modified Fc, do not exhibit agonist activity; whereas the corresponding construct, designated 1208, that contains the dAb from SEQ ID NO:54 has agonist activity. The dAb 019 construct does not have activity when tested with the dAb linked to human serum albumin (HSA).

As described for the constructs above, the TNFR1 inhibitor, linkers, and other components can be those as described above. These include constructs where the TNFR1 inhibitor that is a selective antagonist comprises an antigen-binding fragment that selectively binds and inhibits TNFR1 signaling but not TNFR2 signaling. For example, the antigen-binding fragment that selectively binds and inhibits TNFR1 signaling but not TNFR2 signaling can comprise a domain antibody (dAb), scFv, or Fab fragment. In any of the constructs described herein, the TNFR1 inhibitor comprises an antigen-binding fragment of a human anti-TNFR1 antagonist monoclonal antibody. For example, the human anti-TNFR1 antagonist monoclonal antibody is H398 that comprises SEQ ID NO:678, or ATROSAB, or an antigen binding portion thereof or a sequence having at least 95% sequence identity to SEQ ID NO:31 or 32 or 673 or 678 or an antigen-binding portion thereof that binds to TNFR1. Exemplary of TNFR1 inhibitors are those that comprise a domain antibody (dAb) or antigen binding portion thereof or comprises the sequence of amino acids set forth in any of SEQ ID NOs: 54-672 or a sequence having at least 95% sequence identity thereto that retains TNFR1 inhibitor activity; and/or comprise the scFv set forth in any of SEQ ID NOs:673-678 or variants of these polypeptides having at least 90% or 95% sequence identity thereto that retains TNFR1 inhibitor activity; and/or comprise the Fab set forth in any of SEQ ID NOs:679-682 or a sequence having at least 90% or 95% sequence identity thereto that retains TNFR1 inhibitor or binding activity; and/or comprises the nanobody whose sequence is set forth in SEQ ID NO: 683 or 684 or a sequence having at least 90% or 95% sequence identity thereto that retains TNFR1 inhibitor or binding activity.

Among the TNFR1 inhibitors, are those, for example, that comprise a dominant-negative tumor necrosis factor (DN-TNF) or TNF mutein, such as, for example, a DN-TNF or TNF mutein is a soluble TNF molecule, comprising one or more amino acid replacements that confer selective inhibition of TNFR1 and are selected from among:

V1M, L29S, L29G, L29Y, R31C, R31E, R31N, R32Y, R32W, C69V, A84S, V85T, S86T, Y87H, Q88N, T89Q, I97T, C101A, A145R, E146R, L29S/R32W, L29S/S86T, R32W/S86T, L29S/R32W/S86T, R31N/R32T, R31E/S86T, R31N/R32T/S86T, I97T/A145R, V1M/R31C/C69V/Y87H/C101A/A145R, and A84S/V85T/S86T/Y87H/Q88N/T89Q, with reference to the sequence of soluble TNF, set forth in SEQ ID NO:2. For example, the TNFR1 inhibitor is a TNF mutein that comprises the sequence of residues set forth in any one of SEQ ID NOs:701-703, or a sequence with at least or at least about 90% or 95% sequence identity to the sequence of residues set forth in any one of SEQ ID NOs:701-703 or fragment thereof that retains TNFR1 inhibitor activity. As described herein, the resulting constructs have the additional property, which can be identified by assay, that they do not have TNFR1 agonist activity.

Any of the foregoing constructs provided herein can include a linker, where the linker comprises all or a portion of the hinge sequence of trastuzumab, SCDKTH corresponding to residues 222-227 of SEQ ID NO:26 or up to the full sequence of the hinge region of trastuzumab, that contains or has the sequence EPKSCDKTHTCPPCP (corresponding to residues 219-233 of SEQ ID NO:26), or at least 5, 6, 7, 8, 9, 10, or 11 contiguous residues thereof, or residues ESKYGPPCPPCP, corresponding to residues 212-223 of SEQ ID NO:29, or a sequence having at least 98% or 99% sequence identity thereto that is a linker. For example, the construct can comprise a linker, where the linker comprises the sequence SCDKTH, corresponding to residues 222-227 of SEQ ID NO:26. The constructs can comprise in place of or in addition to another of the linkers, a linker that comprises glycine and serine (GS) residues, a GS linker. Exemplary GS linkers for any of the constructs provided herein include those selected from among (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)ₙ, where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS. Also included are linkers that comprise a GS linker and all or a portion of the hinge sequence of trastuzumab, corresponding to residues EPKSCDKTHTCPPCP (corresponding to residues 219-233 of SEQ ID NO:26), for example, the linker can comprise a GS linker and comprise or contain only the sequence SCDKTH, corresponding to residues 217-222 of SEQ ID NO:31, from the hinge sequence. Such linkers include, for example, those that comprise a GS linker and all or a portion of the hinge sequence of nivolumab, corresponding to residues 212-223 of SEQ ID NO:29.

The constructs herein can contain an activity modifier. The activity modifiers include any described herein, including those described above, and below, and others known to those of skill in the art; the activity modifier alters and activity or property of the construct. The activity modified can be one that is a half-life extending moiety that is an IgG Fc, a polyethylene glycol (PEG) molecule, or human serum albumin (HSA). Examples of an IgG Fc is an IgG1 or IgG4 Fc. The IgG1 Fc can be the Fc of trastuzumab, set forth in SEQ ID NO:27 or a sequence of amino acids having at least 95% sequence identity therewith; the IgG4 Fc can be the Fc of nivolumab, set forth in SEQ ID NO:30 or a sequence of amino acids having at least 95% sequence identity therewith. For example, the IgG1 Fc is the Fc of human IgG1, set forth in SEQ ID NO:10, and the IgG4 Fc is the Fc of human IgG4, set forth in SEQ ID NO:16.

The constructs described herein include those that are TNFR1 inhibitors or comprise a TNFR1 inhibitor(s). These include constructs where the TNFR1 inhibitor is monovalent. These can include linkers, such as where the linker comprises (Gly₄Ser)₃, and/or linkers that comprise (Gly₄Ser)₃ and SCDKTH (residues 217-222 of SEQ ID NO:31); and/or linkers that comprise (Gly₄Ser)₃ and the hinge sequence of trastuzumab, corresponding to residues 219-233 of SEQ ID NO:26; and/or those that comprise (Gly₄Ser)₃ and the hinge sequence of nivolumab, corresponding to residues 212-223 of SEQ ID NO:29. Exemplary of constructs provided herein that inhibit TNFR1 are those that comprise the sequence of residues set forth in any of SEQ ID NOs:704-764, or a construct that inhibits TNFR1 and has a sequence with at least or at least about 95% sequence identity to the sequence of residues set forth in any one of SEQ ID NOs:704-764.

Provided herein are TNFR1 antagonist constructs. These include those where the TNFR1 construct comprises a short FcRn-binding peptide (FcRnBP); and the short FcRn-binding peptide (FcRnBPs) provides for the interaction of the construct with FcRn, and contains 6-25, such as 10-20 amino acid residues, such as for, example, those where the FcRnBP contains 12-20 residues or 15 residues or 16 residues, such as, for example those where the FcRn-binding peptide (FcRnBP) comprises a peptide of any SEQ ID NOs:48-51 or a peptide having at least about 95% sequence identity therewith, or an FcRn-binding peptide (FcRnBP) that includes a peptide of any SEQ ID NOs:48-51.

Other exemplary TNFR1-inhibiting constructs provided herein include constructs that comprise: a) a domain antibody that inhibits TNFR1; b) a linker that increases flexibility; reduces steric effects, or increases solubility; and c) a half-life extending moiety. Included are such constructs where the half-life extending moiety is not a human serum albumin antibody or an unmodified Fc. These constructs include those that are a TNFR1 antagonist, comprising: a) the domain antibody (dAb) of any of SEQ ID NOs:52-672, or the scFv of any of SEQ ID NOs:673-678 or the Fab of any of SEQ ID NOs:679-682, or the nanobody of SEQ ID NO: 683 or 684, or the TNF mutein of any of SEQ ID NOs:685-703; b) a GS linker selected from among (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)ₙ, where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS; and c) a half-life extending moiety that is an IgG Fc. In these constructs, or any provided herein that include one or more of these components, the GS linker can be (GGGGS)₃; and the IgG Fc can be the Fc of trastuzumab or the Fc of nivolumab.

Others of the constructs provided herein that are TNFR1 antagonist constructs include constructs comprising: a) the domain antibody (dAb) of any of SEQ ID NOs:52-672, or the scFv of any of SEQ ID NOs:673-678 or the Fab of any of SEQ ID NOs:679-682, or the nanobody of SEQ ID NO: 683 or 684, or the TNF mutein of any of SEQ ID NOs:685-703; b) a linker selected from among all or a portion of the hinge sequence of trastuzumab and all or a portion of the hinge sequence of nivolumab; and c) a half-life extending moiety that is an IgG Fc. In such constructs, the linker can comprise all or a portion of the hinge sequence of trastuzumab, where the IgG Fc is the Fc of trastuzumab. In other embodiments, the linker can comprise all or a portion of the hinge sequence of nivolumab, where the IgG Fc is the Fc of nivolumab.

Provided are any of the constructs provided herein that is a TNFR1 antagonist construct, comprising:
a) the domain antibody (dAb) of any of SEQ ID NOs:52-672, or the scFv of any of SEQ ID NOs:673-678 or the Fab of any of SEQ ID NOs:679-682, or the nanobody of SEQ ID NO: 683 or 684, or the TNF mutein of any of SEQ ID NOs:685-703;
b) a first linker that is a GS linker selected from among (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)ₙ, where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS;
c) a second linker selected from among all or a portion of the hinge sequence of trastuzumab and all or a portion of the hinge sequence of nivolumab; and
d) a half-life extending moiety that is an IgG Fc.

In some embodiments, these constructs can contain a first linker that is a GS linker is (GGGGS)₃; and a second linker comprises the sequence SCDKTH (residues 217-222 of SEQ ID NO:31); and the IgG Fc is the Fc of trastuzumab. In other embodiments, the first linker is the GS linker is (GGGGS)₃; the second linker comprises all or a portion of the hinge sequence of nivolumab; and the IgG Fc is the Fc of nivolumab.

Provided are the constructs that are TNFR1 antagonists that comprise:
a) the domain antibody (dAb) of any of SEQ ID NOs:52-672, or the scFv of any of SEQ ID NOs:673-678 or the Fab of any of SEQ ID NOs:679-682, or the nanobody of SEQ ID NO: 683 or 684, or the TNF mutein of any of SEQ ID NOs:685-703;
b) a GS linker selected from among (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)ₙ, where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS; and
c) a half-life extending moiety that is a PEG molecule. The GS linker can be any described herein or known to those of skill in the art, such as (GGGGS)₃. The PEG molecule can be one that has a molecular weight of at least 25 kDa, generally at least 30 kDa or more, such as at least 40 kDa or 50 kDa, or 60 kDa, or 80 kDa, or more.

Provided are the constructs that are TNFR1 antagonist constructs, comprising:
a) the domain antibody (dAb) of any of SEQ ID NOs:52-672, or the scFv of any of SEQ ID NOs:673-678, or the Fab of any of SEQ ID NOs:679-682, or the nanobody of SEQ ID NO: 683 or 684, or the TNF mutein of any of SEQ ID NOs:685-703;
b) a GS linker selected from among (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)., where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS; and
c) a half-life extending moiety that is human serum albumin. Exemplary of the linkers are any described herein, such as where the GS linker is (GGGGS)₃.

The primary amino acid sequence of any of the constructs provided herein (those described above, and below) can be optimized or modified to eliminate immunogenic sequences or immunogenic epitopes. For example, in constructs that contain an IgG Fc, the IgG Fc can be modified to comprise one or more of the following modifications: a) a modification(s) to introduce knobs-into-holes; b) a modification(s) to increase or enhance neonatal Fc receptor (FcRn) recycling; and c) a modification(s) to reduce or eliminate immune effector functions. In such constructs and in any that contain an IgG Fc the knob mutation can be selected from among S354C, T366Y, T366W, and T394W by EU numbering; and the hole mutation is selected from among Y349C, T366S, L368A, F405A, Y407T, Y407A, and Y407V by EU numbering. These TNFR1 antagonist constructs can be one where the modification(s) to increase or enhance FcRn recycling is selected from among one or more of: T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering. The TNFR1 antagonist constructs that can be modified to reduce or eliminate immune effector function(s), such as immune effector function(s) that is/are selected from among one or more of complement-dependent cytotoxicity (CDC), antibody-dependent cell-mediated cytotoxicity (ADCC), and antibody-dependent cell-mediated phagocytosis (ADCP).

For example, in these TNFR1 antagonist constructs, the modification(s) to reduce or eliminate immune effector functions are selected from among one or more of: in IgG1: L235E, L234A/L235A, L234E/L235F/P331S, L234F/L235E/P331S, L234A/L235A/P329G, L234A/L235A/G237A/P238S/H268A/A330S/P331S, G236R/L328R, G237A, E318A, D265A, E233P, N297A, N297Q, N297D, N297G, N297G/D265A, A330L, D270A, P329A, P331A, K322A, V264A, and F241A, by EU numbering; and in IgG4: L235E, F234A/L235A, S228P/L235E, and S228P/F234A/L235A, by EU numbering.

The TNFR1 antagonist or multispecific constructs can comprise a central PEG linker moiety; and the construct can comprise a modified Fc region, such as those described above, where Fc region is a modified IgG Fc and the modified IgG Fc comprises one or more of the following modifications:
a) a modification(s) to introduce knobs-into-holes, wherein:
   the knob mutation is selected from among S354C, T366Y, T366W, and T394W by EU numbering; and
   the hole mutation is selected from among Y349C, T366S, L368A, F405A, Y407T, Y407A, and Y407V by EU numbering;
b) a modification(s) to increase or enhance neonatal Fc receptor (FcRn) recycling, wherein the modification is selected from among one or more of: T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering; and
c) a modification(s) to increase or enhance one or more immune effector functions, wherein:
   the immune effector function(s) is/are selected from among one or more of CDC, ADCC and ADCP; and
   the modification(s) to increase or enhance an immune effector function is/are selected from among one or more of:
   in IgG1: S239D, 1332E, S239D/I332E, S239D/A330L/I332E, S298A/E333A/K334A; F243L/R292P/Y300L/V305I/P396L; L235V/F243L/R292P/Y300L/P396L; F243L/R292P/Y300L; L234Y/G236W/S298A in the first heavy chain and S239D/A330L/I332E in the second heavy chain; L234Y/L235Q/G236W/S239M/H268D/D270E/S298A in the first heavy chain and D270E/K326D/A330M/K334E in the second heavy chain; A327Q/P329A; D265A/S267A/H268A/D270A/K326A/S337A; T256A/K290A/S298A/E333A/K334A; G236A; G236A/1332E; G236A/S239D/I332E; G236A/S239D/A330L/I332E; introduction of a biantennary glycan at residue N297; introduction of an afucosylated glycan at residue N297; K326W; K326A; E333A; K326A/E333A; K326W/E333S; K326M/E333S; K222W/T223W; K222W/T223W/H224W; D221W/K222W; C220D/D221C; C220D/D221C/K222W/T223W; H268F/S324T; S267E; H268F; S324T; S267E/H268F/S324T; G236A/I332E/S267E/H268F/S324T; E345R; and E345R/E430G/S440Y; by EU numbering.

In some embodiments, of any of the constructs that comprises an Fc region, the construct can comprise an IgG1 Fc that comprises one or more modifications to increase binding to the inhibitory Fcγ receptor (FcγR) FcγRIIb. For example, the modification or modifications that increase binding to FcγRIIb is/are selected from among one or more of S267E, N297A, L328F, L351S, T366R, L368H, P395K, S267E/L328F and L351S/T366R/L368H/P395K, by EU numbering.

The constructs can include linker, such as:
a) a GS linker selected from among (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)ₙ, where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS; and
b) an activity modifier that is a half-life extending moiety that is an IgG Fc modified as detailed herein. In exemplary embodiments, the GS linker can be (GGGGS)₃; and the IgG Fc is the Fc of trastuzumab or the Fc of nivolumab.
c) a second linker selected from among all or a portion of the hinge sequence of trastuzumab and all or a portion of the hinge sequence of nivolumab; and
d) an activity modifier that is a half-life extending moiety that is an IgG Fc.

Exemplary of such constructs are those in which the first GS linker is (GGGGS)₃, and the second linker comprises the sequence SCDKTH (residues 217-222 of SEQ ID NO:31); and the IgG Fc is the Fc of trastuzumab. In other embodiments, the first linker is (GGGGS)₃, the second linker comprises all or a portion of the hinge sequence of nivolumab; and the IgG Fc is the Fc of nivolumab.

Provided are TNFR1 antagonist constructs, TNFR1antagonsit-TNFR2 agonist constructs, where the IgG Fc is a monomer or a dimer. The constructs provided herein can comprise a dAb (or a Vhh). The constructs can comprise a Vhh single chain or double chain containing a dAb. These constructs can contain HSA linked to the dAb directly or via a linker. They HSA and dAb can be linked in any order, such as the C-terminus of the dAb linked directly or via a linker, such as any described above, to the N-terminus of HSA. Exemplary of such constructs are those that comprise:
a) residues 20-732, which is the dAb Dom1h-131-206 of SEQ ID NO:59, linked via a linker to HSA, as set forth in SEQ ID NO:1475, or a construct having at least 95%, 96%, 97%, 98%, 99% sequence identity to the construct of SEQ ID NO:1475 or to residues 20-732 of SEQ ID NO:1475 and having TNFR1 antagonist activity; or
b) a dAb set forth in in any of SEQ ID NOs: 53-83 and 503-671, and variants thereof having at least 95%, 96%, 97%, 98%, 99% sequence identity thereto, whereby the construct has TNFR1 antagonist activity; or
c) a dAb that has the sequence set forth in any of SEQ ID NOs:57-59 and variants thereof have at least 95% sequence identity thereto, whereby the construct has TNFR1 antagonist activity; or
d) the dAb is designated DOM1 h-131-206 of SEQ ID NO:59 and variants thereof that have at least 95%, 96%, 97%, 98%, 99% sequence identity thereto, and have TNFR1 antagonist activity; or
e) combinations of any of a)-d); or
f) humanized sequences of any of a)-f) or where a sufficient portion of the construct for administration to a human is humanized, where a sufficient portion is sufficient to eliminate or reduce any immune response to the construct when administered to a human.

The constructs provided herein that are TNFR1 constructs can further comprise a TNFR2 agonist or the construct can be a TNFR2 agonist construct. In the constructs that comprise a TNFR2 agonist, the TNFR2 agonist can be modified to eliminate sequences of amino acids or epitopes that are immunogenic in the subject to be treated, such as for administration to a human subject. In the constructs that contain TNFR2 agonist, it can be a TNFR2-selective agonist. These constructs can comprise a modified IgG Fc. For example, the IgG Fc can comprise one or more of the following modifications:
a) a modification(s) to introduce knobs-into-holes;
b) a modification(s) to increase or enhance neonatal Fc receptor (FcRn) recycling; and
c) a modification(s) to reduce or eliminate immune effector functions, selected from among one or more of complement-dependent cytotoxicity (CDC), antibody-dependent cell-mediated cytotoxicity (ADCC) and antibody-dependent cell-mediated phagocytosis (ADCP). Exemplary of such modifications are:
a) a modification(s) to introduce knobs-into-holes are selected from:
one or more knob mutations selected from among S354C, T366Y, T366W, and T394W by EU numbering; and
one or more hole mutations selected from among Y349C, T366S, L368A, F405A, Y407T, Y407A, and Y407V by EU numbering, whereby the Fc forms a dimer;
b) the modification(s) to increase or enhance FcRn recycling is selected from among one or more of T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering; and
c) the modification(s) to reduce or eliminate immune effector functions are selected from among one or more of:
   in IgG1: L235E, L234A/L235A, L234E/L235F/P331S, L234F/L235E/P331S, L234A/L235A/P329G, L234A/L235A/G237A/P238S/H268A/A330S/P331S, G236R/L328R, G237A, E318A, D265A, E233P, N297A, N297Q, N297D, N297G, N297G/D265A, A330L, D270A, P329A, P331A, K322A, V264A, and F241A, by EU numbering; and
   in IgG4: L235E, F234A/L235A, S228P/L235E, and S228P/F234A/L235A, by EU numbering.

Constructs provided herein include TNFR2 agonist constructs that contain a modified IgG Fc, where the IgG Fc comprises one or more of the following modifications:
a) one or more modification(s) to introduce knobs-into-holes, wherein:
   the knob mutation is selected from among S354C, T366Y, T366W, and T394W by EU numbering; and
   the hole mutation is selected from among Y349C, T366S, L368A, F405A, Y407T, Y407A, and Y407V by EU numbering;
b) a modification(s) to increase or enhance neonatal Fc receptor (FcRn) recycling, wherein the modification is selected from among one or more of:
   T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering; and
c) a modification(s) to increase or enhance immune effector functions, wherein:
   the immune effector functions are selected from among one or more of CDC, ADCC and ADCP; and
   the modification(s) in to increase or enhance immune effector functions is selected from among one or more of:
      in IgG1: S239D, 1332E, S239D/I332E, S239D/A330L/I332E, S298A/E333A/K334A; F243L/R292P/Y300L/V305I/P396L; L235V/F243L/R292P/Y300L/P396L; F243L/R292P/Y300L; L234Y/G236W/S298A in the first heavy chain and S239D/A330L/I332E in the second heavy chain; L234Y/L235Q/G236W/S239M/H268D/D270E/S298A in the first heavy chain and D270E/K326D/A330M/K334E in the second heavy chain; A327Q/P329A; D265A/S267A/H268A/D270A/K326A/S337A; T256A/K290A/S298A/E333A/K334A; G236A; G236A/I332E; G236A/S239D/I332E; G236A/S239D/A330L/I332E; introduction of a biantennary glycan at residue N297; introduction of an afucosylated glycan at residue N297; K326W; K326A; E333A; K326A/E333A; K326W/E333S; K326M/E333S; K222W/T223W; K222W/T223W/H224W; D221W/K222W; C220D/D221C; C220D/D221C/K222W/T223W; H268F/S324T; S267E; H268F; S324T; S267E/H268F/S324T; G236A/I332E/S267E/H268F/S324T; E345R; and E345R/E430G/S440Y; by EU numbering.

The constructs provided herein that are TNFR2 agonist construct can comprise a modified IgG1 Fe, such as where the Fc is modified to increase binding to the inhibitory Fcγ receptor (FcγR) FcγRIIb, which can include modifications that increase binding to FcγRIIb. Exemplary of such modifications are those selected from among one or more of S267E, N297A, L328F, L351S, T366R, L368H, P395K, S267E/L328F and L351S/T366R/L368H/P395K, by EU numbering.

These constructs comprise a modified Fc, wherein the IgG Fc comprises one or more of the following modifications:
a) a modification(s) to introduce knobs-into-holes;
b) a modification(s) to increase or enhance neonatal Fc receptor (FcRn) recycling; and
c) a modification(s) to reduce or eliminate immune effector functions.

Exemplary of the Fc that comprise knobs-into-holes modifications are:
the knob mutation is selected from among one or more of S354C, T366Y, T366W, and T394W by EU numbering; and
the hole mutation is selected from among one or more of Y349C, T366S, L368A, F405A, Y407T, Y407A, and Y407V by EU numbering.

Other examples are multi-specific constructs that comprise an Fc, such as where the Fc comprises modifications to increase or enhance FcRn recycling is/are selected from among one or more of T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering. The Fc can comprise modifications to immune effector functions that are selected from among one or more of complement-dependent cytotoxicity (CDC), antibody-dependent cell-mediated cytotoxicity (ADCC) and antibody-dependent cell-mediated phagocytosis (ADCP). The Fc can comprise modification(s) to reduce or eliminate immune effector functions in IgG1 and/or IgG4:
in IgG1: L235E, L234A/L235A, L234E/L235F/P331S, L234F/L235E/P331S, L234A/L235A/P329G, L234A/L235A/G237A/P238S/H268A/A330S/P331S, G236R/L328R, G237A, E318A, D265A, E233P, N297A, N297Q, N297D, N297G, N297G/D265A, A330L, D270A, P329A, P331A, K322A, V264A, and F241A, by EU numbering; and/or
in IgG4: L235E, F234A/L235A, S228P/L235E, and S228P/F234A/L235A, by EU numbering.

The IgG Fc can comprise one or more of the following modifications:
a) a modification(s) to introduce knobs-into-holes, wherein:
   the knob mutation is selected from among one or more of S354C, T366Y, T366W, and T394W by EU numbering; and
   the hole mutation is selected from among one or more of Y349C, T366S, L368A, F405A, Y407T, Y407A, and Y407V by EU numbering;
b) a modification(s) to increase or enhance neonatal Fc receptor (FcRn) recycling, wherein the modification is selected from among one or more of:
   T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering; and
c) a modification(s) to increase or enhance immune effector functions, wherein:
   the immune effector functions are selected from among one or more of CDC, ADCC and ADCP; and
   the modification(s) in to increase or enhance immune effector functions is selected from among one or more of:
      in IgG1: S239D, 1332E, S239D/I332E, S239D/A330L/I332E, S298A/E333A/K334A; F243L/R292P/Y300L/V305I/P396L; L235V/F243L/R292P/Y300L/P396L; F243L/R292P/Y300L; L234Y/G236W/S298A in the first heavy chain and S239D/A330L/I332E in the second heavy chain; L234Y/L235Q/G236W/S239M/H268D/D270E/S298A in the first heavy chain and D270E/K326D/A330M/K334E in the second heavy chain; A327Q/P329A; D265A/S267A/H268A/D270A/K326A/S337A; T256A/K290A/S298A/E333A/K334A; G236A; G236A/I332E; G236A/S239D/I332E; G236A/S239D/A330L/I332E; introduction of a biantennary glycan at residue N297; introduction of an afucosylated glycan at residue N297; K326W; K326A; E333A; K326A/E333A; K326W/E333 S; K326M/E333 S; K222W/T223W; K222W/T223W/H224W; D221W/K222W; C220D/D221C; C220D/D221C/K222W/T223W; H268F/S324T; S267E; H268F; S324T; S267E/H268F/S324T; G236A/1332E/S267E/H268F/S324T; E345R; and E345R/E430G/S440Y; by EU numbering.

Other of such multi-specific constructs are those where: the construct that comprises an IgG1 Fc that is modified to increase binding to the inhibitory Fcγ receptor (FcγR) FcγRIIb. Exemplary of such are those where the modifications that increase binding to FcγRIIb are selected from among one or more of S267E, N297A, L328F, L351S, T366R, L368H, P395K, S267E/L328F and L351S/T366R/L368H/P395K, by EU numbering.

Also provided are constructs that are a multi-specific TNFR1 antagonist/TNFR2 agonist, the TNFR1 inhibitor (antagonist) is monovalent; and the TNFR2 agonist is monovalent. Also provided are multi-specific constructs that are a multi-specific TNFR1 antagonist/TNFR2 agonist constructs, where the TNFR1 antagonist is monovalent; and the TNFR2 agonist is bivalent. As with all of the constructs provided the TNFR1 antagonist does not exhibit TNFR1 agonist activity.

In some embodiments, the multi-specific constructs are multi-specific TNFR1 antagonist/TNFR2 agonist constructs, where:
a) the TNFR1 antagonist is selected from:
   i) an antigen-binding fragment of a human anti-TNFR1 antagonist monoclonal antibody selected from H398 or ATROSAB; or
   ii) the domain antibody (dAb) of any of SEQ ID NOs:52-672, or the scFv of any of SEQ ID NOs:673-678, or the Fab of any of SEQ ID NOs:679-682, or the nanobody of SEQ ID NO: 683 or 684, or the TNF mutein of any of SEQ ID NOs:701-703, or a sequence with at least or at least about 95% sequence identity thereto; or
   iii) a dominant-negative tumor necrosis factor (DN-TNF) or TNF mutein comprising a soluble TNF molecule, with one or more amino acid replacements that confer selective inhibition of TNFR1 and are selected from among:
      VIM, L29S, L29G, L29Y, R31C, R31E, R31N, R32Y, R32W, C69V, A84S, V85T, S86T, Y87H, Q88N, T89Q, I97T, C101A, A145R, E146R, L29S/R32W, L29S/S86T, R32W/S86T, L29S/R32W/S86T, R31N/R32T, R31E/S86T, R31N/R32T/S86T, I97T/A145R, V1M/R31C/C69V/Y87H/C101A/A145R, and A84S/V85T/S86T/Y87H/Q88N/T89Q, with reference to the sequence of soluble TNF, set forth in SEQ ID NO:2;
b) the linker is a branched chain PEG molecule that is at least or at least about 30 kDa in size; and
c) the TNFR2 agonist is selected from:
   i) an antigen-binding fragment that binds to one or more epitopes within human TNFR2 that is selected from among the epitopes set forth in SEQ ID NOs:839-865, 1202 and 1204; or
   ii) an antigen-binding fragment of an agonistic human anti-TNFR2 antibody selected from MR2-1 or MAB2261; or
   iii) a TNFR2-selective TNF mutein that is a soluble TNF variant comprising one or more TNFR2-selective mutations selected from among K65W, D143Y, D143F, D143N, D143E, D143W, D143V, A145R, A145H, A145K, A145F, A145W, E146Q, E146H, E146K, E146N, D143N/A145R, A145R/S147T, Q88N/T89S/A145S/E146A/S147D, Q88N/A1451/E146G/S147D, A145H/E146S/S147D, A145H/S147D, L29V/A145D/E146D/S147D, A145N/E146D/S147D, A145T/E146S/S147D, A145Q/E146D/S147D, A145T/E146D/S147D, A145D/E146G/S147D, A145D/S147D, A145K/E146D/S147T, A145R/E146T/S147D, A145R/S147T, E146D/S147D, D143V/F144L/A145S, S95C/G148C, and D143V/A145S, with reference to SEQ ID NO:2; or
   iv) a single-chain TNFR2-selective TNF mutein trimer, comprising the mutations D143N/A145R, wherein the TNF muteins are linked by (GGGGS)ₙ, where n=1-5, or all or a portion of the stalk region of TNF (SEQ ID NO:812); or
   v) a TNFR2-selective agonist comprising the formula:
   MD-L1-TNFmut-L2-TNFmut-L3-TNFmut (Formula II); or
   TNFmut-L1-TNFmut-L2-TNFmut-L3-MD (Formula III);
   whereby MD is a multimerization domain; TNFmut is a TNFR2-selective TNF mutein; and L1, L2 and L3 are linkers that can be the same or different, and wherein:
      the MD is selected from EHD2 (SEQ ID NO:808), MHD2 (SEQ ID NO:811), the trimerization domain of chicken tenascin C (TNC) (residues 110-139 of SEQ ID NO:804; SEQ ID NO:805), or the trimerization domain of human TNC (residues 110-139 of SEQ ID NO:806, SEQ ID NO:807);
      L1, L2 and L3 each are (GGGGS)ₙ, where n=1-5, or all or a portion of the stalk region of TNF (SEQ ID NO:812), or a mixture thereof; and
      the TNF muteins comprise the TNFR2-selective mutations D143N/A145R.

Also provided are multi-specific constructs where each of the TNFR1 antagonist and TNFR2 agonist is monovalent. Also provided are such constructs where the TNFR1 antagonist is monovalent, and the TNFR2 agonist is bivalent.

The constructs provided herein can be used for treatments and uses for treatment of various diseases, disorders, and conditions. Provided are the multi-specific constructs that are multi-specific TNFR1 antagonist/TNFR2 agonist, for use for the treatment of a chronic inflammatory, autoimmune, neurodegenerative, demyelinating or respiratory disease or disorder, or a disease, condition or disorder characterized by overexpression of TNF or deregulated TNFR1 signaling in its etiology. Uses of multi-specific TNFR1 antagonist/TNFR2 agonist constructs for the treatment of a chronic inflammatory, autoimmune, neurodegenerative, demyelinating or respiratory disease or disorder, or a disease, condition or disorder characterized by overexpression of TNF or deregulated TNFR1 signaling in its etiology are provided.

Also provided are compositions, comprising a construct of any of the constructs provided herein in a pharmaceutically acceptable carrier or vehicle. These compositions can be used for or in methods of treatment of diseases, disorders, and conditions, such as, but not limited to, a chronic inflammatory, autoimmune, neurodegenerative, demyelinating or respiratory disease or disorder, and a disease, condition or disorder characterized by overexpression of TNF or deregulated TNFR1 signaling in its etiology. Exemplary diseases, disorders, and conditions, are inflammatory, autoimmune, neurodegenerative, demyelinating or respiratory diseases or disorders, and diseases, disorders, and conditions characterized by overexpression of TNF or deregulated TNFR1 signaling in its etiology. These include diseases, disorders, and conditions selected from: rheumatoid arthritis (RA), psoriasis, psoriatic arthritis, juvenile idiopathic arthritis (JIA), spondyloarthritis, ankylosing spondylitis, Crohn's disease, ulcerative colitis, inflammatory bowel disease (IBD), uveitis, fibrotic diseases, endometriosis, lupus, multiple sclerosis (MS), congestive heart failure, cardiovascular disease, myocardial infarction (MI), atherosclerosis, metabolic diseases, cytokine release syndrome, septic shock, sepsis, acute respiratory distress syndrome (ARDS), severe acute respiratory syndrome (SARS), SARS-CoV-2, influenza, acute and chronic neurodegenerative diseases, demyelinating diseases and disorders, stroke, Alzheimer's disease, Parkinson's disease, Behget's disease, Dupuytren's disease, Tumor Necrosis Factor Receptor-Associated Periodic Syndrome (TRAPS), pancreatitis, type I diabetes, chronic obstructive pulmonary disease (COPD), chronic bronchitis, emphysema, graft rejection, graft versus host disease (GvHD), lung inflammation, pulmonary diseases and conditions, asthma, cystic fibrosis, idiopathic pulmonary fibrosis, acute fulminant viral or bacterial infections, pneumonia, genetically inherited diseases with TNF/TNFR1 as the causative pathologic mediator, periodic fever syndrome, or cancer. In particular, constructs provided herein, such as, but not limited to, the TNFR1 antagonist constructs, can be used in uses, methods of treatment, and compositions for the treatment of rheumatoid arthritis.

Among the constructs, constituent chains, and other variants provided herein are the following. Provided are constructs that are tumor necrosis factor receptor 1 (TNFR1) antagonist constructs of formula 1:

(TNFR1 inhibitor)ₙ-linkerₚ-(activity modifier)_{q}, wherein:
each of n and q is an integer, n is 1 or 2, and q is 1;
p is 0, 1, 2 or 3;
a TNFR1 inhibitor is a molecule that binds TNFR1 to inhibit (antagonize) TNFR1;
an activity modifier is a moiety that modulates or alters the activity or the pharmacological property of the construct compared to the construct in the absence of the activity modifier;
a linker increases flexibility of the construct, and/or moderates or reduces steric effects of the construct or its interaction with a receptor, and/or increases solubility of the construct in aqueous medium;
the construct is a fusion protein that comprises a TNFR1 inhibitor that comprises a domain antibody (dAb);
the dAb comprises a dAb selected from those with a sequence set forth in any of SEQ ID NOs: 54-672, wherein the resulting construct, when bivalent where n is 2, does not have TNRF1 agonist activity;
when n is 2 the construct is bivalent and the domain antibody (dAb) is selected so that the construct has antagonist activity, but does not have agonist activity;
the activity modifier is a modified Fc that has one or more of
   a) mutations to increase serum half-life;
   mutations the Fc for the purpose of silencing the antibody effector function; and, when n=1 and q=2, the fc has a modification(s) to introduce knobs-into-holes;
b) a modification(s) to increase or enhance neonatal Fc receptor (FcRn) recycling; and
c) a modification(s) to reduce or eliminate immune effector functions.

Included are constructs in which the TNFR1 inhibitor comprises a domain antibody (dAb), or antigen-binding portion thereof or comprises the sequence of amino acids set forth in any of SEQ ID NOs: 54-672, or a sequence having at least 95% sequence identity thereto that retains TNFR1 inhibitor activity; and the dAb binds to the same epitope as the dAb of SEQ ID NO:59, and/or is one that, when n is 2 does not have TNFR1 agonist activity.

Included are those in which the dAb is one that binds to the same epitope on TNRF1 as the dAb of SEQ ID NO:59 binds. These include the dAbs whose sequence are set forth set forth in any of SEQ ID NOs: 55-84, 86-88, 450, and 495-498, which bind to the same epitope as dAb of SEQ ID NO:59. Also included are those in which the dAb portion comprises CDRs that have at least 95% sequence identity to HCDR1, HCDR2, and HCDR3 (SEQ ID NOs: 1510-1512, respectively) of DOM1h-131-511 (SEQ ID NO: 60), which binds domain 3 of the TNFR1 extracellular domain (ECD). FIG. 18 shows the complementary-determining regions (CDRs) for the dAb DOM1 h-311-511 and its variant 206.

As shown herein, the dAb, designated EN1206 (DOM1 h-131-206; SEQ ID NO: 59), binds to the domain 3 of the TNFR1 extracellular domain (ECD). As assayed herein it binds with an affinity of about 300-500 pM as measured by SPR.

The Fc portions of the constructs are modified to increase half-life and to reduce or eliminate immune effector functions, such as, one or more of complement-dependent cytotoxicity (CDC), antibody-dependent cell-mediated cytotoxicity (ADCC) and antibody-dependent cell-mediated phagocytosis (ADCP).

The constructs optionally include linkers between components. Linkers include, but are not limited to, chemical linkers, polypeptide linkers, and combinations thereof.

Provided are polypeptide chain selected from:
a) SEQ ID NO:1488, which is an Fc only comprising T366S,L368A,Y407V the hole, M252Y, S254T, T256E for increased half-life, 234A,L235A,P329G for minimizing, eliminating, or reducing Fc effector function, and includes a leader sequence, MGWSCIILFLVATATGVHS (SEQ ID NO:1509), that is cleaved off in a cell in which is the polypeptide is expressed and comprises the sequence:

MGWSCIILFLVATATGVHSDKTHTCPPCPAPEAAGGPSVFLFPPKPKDTL
YITREPEVTCVVVDVSHEDPEVKFNWYVDGVEVHNAKTKPREEQYNSTYR
VVSVLTVLHQDWLNGKEYKCKVSNKALGAPIEKTISKAKGQPREPQVYTL
PPSRDELTKNQVSLSCAVKGFYPSDIAVEWESNGQPENNYKTTPPVLDSD
GSFFLVSKLTVDKSRWQQGNVFSCSVMHEALHNHYTQKSLSLSPGK;

b) SEQ ID NO:1489, which comprises a dAb and Fc, where the Fc comprises the modifications T366W to form a knob, M252Y, S254T, T256E for increased half-life, and L234A,L235A,P329G for minimizing, reducing, or eliminating Fc effector function, and the chain includes a leader sequence, MGWSCIILFLVATATGVHS (SEQ ID NO:1509), that is cleaved off in a cell in which is the polypeptide is expressed:

MGWSCIILFLVATATGVHSEVQLLESGGGLVQPGGSLRLSCAASGFTFAH
ETMVWVRQAPGKGLEWVSHIPPDGQDPFYADSVKGRFTISRDNSKNTLYL
QMNSLRAEDTAVYHCALLPKRGPWFDYWGQGTLVTVSSDKTHTCPPCPAP
EAAGGPSVFLFPPKPKDTLYITREPEVTCVVVDVSHEDPEVKFNWYVDGV
EVHNAKTKPREEQYNSTYRVVSVLTVLHQDWLNGKEYKCKVSNKALGAPI
EKTISKAKGQPREPQVYTLPPSRDELTKNQVSLWCLVKGFYPSDIAVEWE
SNGQPENNYKTTPPVLDSDGSFFLYSKLTVDKSRWQQGNVFSCSVMHEAL
HNHYTQKSLSLSPGK;

c) SEQ ID NO:1505, which is an Fe with T366S,L368A,Y407V to form a hole, M252Y, S254T, T256E for increased half-life, L234A,L235A,P329G for minimizing, reducing, or eliminating Fc effector function, and comprises the sequence:

DKTHTCPPCPAPEAAGGPSVFLFPPKPKDTLYITREPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPGK;

and
d) SEQ ID NO:1506, which comprises a dAb and Fc, where the Fc comprises T366W to form a knob, M252Y, S254T, T256E for increased half-life, and L234A, L235A, P329G for minimizing, reducing, or eliminating Fc effector function, and comprises the sequence:

EVQLLESGGGLVQPGGSLRLSCAASGFTFAHETMVWVRQAPGKGLEWVSH
IPPDGQDPFYADSVKGRFTISRDNSKNTLYLQMNSLRAEDTAVYHCALLP
KRGPWFDYWGQGTLVTVSSDKTHTCPPCPAPEAAGGPSVFLFPPKPKDTL
YITREPEVTCVVVDVSHEDPEVKFNWYVDGVEVHNAKTKPREEQYNSTYR
VVSVLTVLHQDWLNGKEYKCKVSNKALGAPIEKTISKAKGQPREPQVYTL
PPSRDELTKNQVSLWCLVKGFYPSDIAVEWESNGQPENNYKTTPPVLDSD
GSFFLYSKLTVDKSRWQQGNVFSCSVMHEALHNHYTQKSLSLSPGK.

Exemplary chains for forming the monovalent construct comprising two chains, where one chain comprises EN1206 monovalent and the Fc with hole, and the other chain comprises EN1206 and the Fc with knob, include those where: each chain comprises the leader sequence MGWSCIILFLVATATGVHS that is cleaved when expressed in a cell and is not present in the resulting construct; and each chain, when expressed, comprises the leader sequence and comprises the sequences:
a) a first chain that is an Fc with modified residues T366S,L368A,Y407V to form a hole, M252Y, S254T, T256E for increasing half-life and L234A, L235A, P329G for minimizing, reducing, or eliminating Fc effector function and having the sequence (SEQ ID NO:1488):

MGWSCIILFLVATATGVHSDKTHTCPPCPAPEAAGGPSVFLFPPKPKDTL
YITREPEVTCVVVDVSHEDPEVKFNWYVDGVEVHNAKTKPREEQYNSTYR
VVSVLTVLHQDWLNGKEYKCKVSNKALGAPIEKTISKAKGQPREPQVYTL
PPSRDELTKNQVSLSCAVKGFYPSDIAVEWESNGQPENNYKTTPPVLDSD
GSFFLVSKLTVDKSRWQQGNVFSCSVMHEALHNHYTQKSLSLSPGK,

wherein MGWSCIILFLVATATGVHS is the leader sequence; and
b) the second chain that comprises the dAb and a modified Fc with T366W to form a knob, M252Y, S254T, T256E for increasing half-life, and L234A, L235A, P329G minimizing, reducing, or eliminating Fc effector function and having the sequence (SEQ ID NO:1489):

MGWSCIILFLVATATGVHSEVQLLESGGGLVQPGGSLRLSCAASGFTFAH
ETMVWVRQAPGKGLEWVSHIPPDGQDPFYADSVKGRFTISRDNSKNTLYL
QMNSLRAEDTAVYHCALLPKRGPWFDYWGQGTLVTVSSDKTHTCPPCPAP
EAAGGPSVFLFPPKPKDTLYITREPEVTCVVVDVSHEDPEVKFNWYVDGV
EVHNAKTKPREEQYNSTYRVVSVLTVLHQDWLNGKEYKCKVSNKALGAPI
EKTISKAKGQPREPQVYTLPPSRDELTKNQVSLWCLVKGFYPSDIAVEWE
SNGQPENNYKTTPPVLDSDGSFFLYSKLTVDKSRWQQGNVFSCSVMHEAL
HNHYTQKSLSLSPGK.

wherein MGWSCIILFLVATATGVHS (SEQ ID NO:1509) is a leader sequence present when the chain is expressed but is not present in the two-chain monovalent construct.

Other chains, such as those for bivalent constructs with two identical chains include those that have comprise the sequence

(SEQ ID NO: 1507)
EVQLLESGGGLVQPGGSLRLSCAASGFTFAHETMVWVRQAPGKGLEWVSH
IPPDGQDPFYADSVKGRFTISRDNSKNTLYLQMNSLRAEDTAVYHCALLP
KRGPWFDYWGQGTLVTVSSDKTHTCPPCPAPEAAGGPSVFLFPPKPKDTL
YITREPEVTCVVVDVSHEDPEVKFNWYVDGVEVHNAKTKPREEQYNSTYR
VVSVLTVLHQDWLNGKEYKCKVSNKALGAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK.

Constructs comprising two chains, include those in which:
a) one chain comprises the polypeptide (SEQ ID NO:1505)

DKTHTCPPCPAPEAAGGPSVFLFPPKPKDTLYITREPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPGK,
with modified residues T366S, L368A, Y407V to form
a hole, M252Y, S254T, T256E for increasing half-
life and L234A, L235A, P329G that reduce or
eliminate Fc effector function;

and
b) the second chain comprises the polypeptide (SEQ ID NO:1506)

EVQLLESGGGLVQPGGSLRLSCAASGFTFAHETMVWVRQAPGKGLE
WVSHIPPDGQDPFYADSVKGRFTISRDNSKNTLYLQMNSLRAEDTA
VYHCALLPKRGPWFDYWGQGTLVTVSSDKTHTCPPCPAPEAAGGPS
VFLFPPKPKDTLYITREPEVTCVVVDVSHEDPEVKFNWYVDGVEVH
NAKTKPREEQYNSTYRVVSVLTVLHQDWLNGKEYKCKVSNKALGAP
IEKTISKAKGQPREPQVYTLPPSRDELTKNQVSLWCLVKGFYPSDI
AVEWESNGQPENNYKTTPPVLDSDGSFFLYSKLTVDKSRWQQGNVF
SCSVMHEALHNHYTQKSLSLSPGK,

which comprises a dAb linked to the Fe with the hole and modified to have increased half-life and reduced or eliminated Fc effector function as in the first chain.

Exemplary constructs are those designated EN1206-MV or EN1206-BV that comprises the dAb of SEQ ID NO:59 and the modified Fc that comprises M252Y, S254T, T256E for increasing half-life and L234A,L235A,P329G for minimizing, reducing, or eliminating Fc effector function, or a construct that comprises the dAb of any of SEQ ID NOs: 55-58, 60-84, 86-88, 450, or 495-498, in place of the dAb of SEQ ID NO:59. The construct designated EN1206-BV two chains where each chain comprises the sequence (SEQ ID NO:1507):

EVQLLESGGGLVQPGGSLRLSCAASGFTFAHETMVWVRQAPGKGLE
WVSHIPPDGQDPFYADSVKGRFTISRDNSKNTLYLQMNSLRAEDTA
VYHCALLPKRGPWFDYWGQGTLVTVSSDKTHTCPPCPAPEAAGGPS
VFLFPPKPKDTLYITREPEVTCVVVDVSHEDPEVKFNWYVDGVEVH
NAKTKPREEQYNSTYRVVSVLTVLHQDWLNGKEYKCKVSNKALGAP
IEKTISKAKGQPREPQVYTLPPSRDELTKNQVSLTCLVKGFYPSDI
AVEWESNGQPENNYKTTPPVLDSDGSFFLYSKLTVDKSRWQQGNVF
SCSVMHEALHNHYTQKSLSLSPGK.

In contrast provided is a bivalent construct designated EN1208-BV, wherein each chain comprises the sequence:

(SEQ ID NO: 1508)
EVQLLESGGGLVQPGGSLRLSCAASGFTFDKYSMGWVRQAPGKGLE
WVSQISDTADRTYYAHAVKGRFTISRDNSKNTLYLQMNSLRAEDTA
VYYCAIYTGRWVPFEYWGQGTLVTVSSDKTHTCPPCPAPEAAGGPS
VFLFPPKPKDTLYITREPEVTCVVVDVSHEDPEVKFNWYVDGVEVH
NAKTKPREEQYNSTYRVVSVLTVLHQDWLNGKEYKCKVSNKALGAP
IEKTISKAKGQPREPQVYTLPPSRDELTKNQVSLTCLVKGFYPSDI
AVEWESNGQPENNYKTTPPVLDSDGSFFLYSKLTVDKSRWQQGNVF
SCSVMHEALHNHYTQKSLSLSPGK.

Provided is a construct that is a bivalent construct comprising two chains, comprising a dAb and a modified Fc, wherein the leader sequence MGWSCIILFLVATATGVHS is cleaved upon expression in a cell; and is not present in the resulting construct:

(SEQ ID NO: 1490)
MGWSCIILFLVATATGVHSEVQLLESGGGLVQPGGSLRLSCAASGF
TFAHETMVWVRQAPGKGLEWVSHIPPDGQDPFYADSVKGRFTISRD
NSKNTLYLQMNSLRAEDTAVYHCALLPKRGPWFDYWGQGTLVTVSS
DKTHTCPPCPAPEAAGGPSVFLFPPKPKDTLYITREPEVTCVVVDV
SHEDPEVKFNWYVDGVEVHNAKTKPREEQYNSTYRVVSVLTVLHQD
WLNGKEYKCKVSNKALGAPIEKTISKAKGQPREPQVYTLPPSRDEL
TKNQVSLTCLVKGFYPSDIAVEWESNGQPENNYKTTPPVLDSDGSF
FLYSKLTVDKSRWQQGNVFSCSVMHEALHNHYTQKSLSLSPGK

The constructs herein include those that comprise a dAb, an optional linker, and a modified Fe, wherein the dAb is selected from

a) DOM1h-576-208 SEQ ID NO: 54:
EVQLLESGGGLVQPGGSLRLSCAASGFTFDKYSMGWVRQAPGKGLE
WVSQISDTADRTYYAHAVKGRFTISRDNSKNTLYLQMNSLRAEDTA
VYYCAIYTGRWVPFEYWGQGTLVTVSS;
or
b) DOM1h-131-206 SEQ ID NO: 59:
EVQLLESGGGLVQPGGSLRLSCAASGFTFAHETMVWVRQAPGKGLE
WVSHIPPDGQDPFYADSVKGRFTISRDNSKNTLYLQMNSLRAEDTA
VYHCALLPKRGPWFDYWGQGTLVTVSS.

Those that comprise SEQ ID NO:59 have TNFR1 antagonist activity, but do not exhibit agonist activity.

Provided are constructs that comprise any of the chains provided herein. Thes include those in which the activity modifier is a modified Fc that has one or more of, generally at least b) and c):
a) a modification(s) to introduce knobs-into-holes;
b) a modification(s) to increase or enhance neonatal Fc receptor (FcRn) recycling; and
c) a modification(s) to reduce or eliminate immune effector functions, selected from among one or more of complement-dependent cytotoxicity (CDC), antibody-dependent cell-mediated cytotoxicity (ADCC) and antibody-dependent cell-mediated phagocytosis (ADCP). For example, the Fc is modified to have reduced or no ADCC activity and/or reduced or no CDC activity.

Also provided are constructs in which the TNFR1 inhibitor inhibits a TNFR1 activity, but does not antagonize tumor necrosis factor receptor 2 (TNFR2) activity. TNFR1 inhibition can include inhibition of TNFR1 signaling and/or ligand binding to TNFR1 or other such activities.

Also provided herein are constructs that are TNFR1 antagonist constructs that comprise a TNFR1 inhibitor that specifically targets and inhibits TNFR1, but that does not antagonize TNFR2. The constructs can prevent transient activation of TNFR1 via receptor clustering.

Among the constructs are those that comprise a linker between the dAb and Fc portions. Exemplary linkers are selected from among:
a) a linker that comprises all or a portion of the hinge sequence of trastuzumab, SCDKTH (corresponding to residues 222-227 of SEQ ID NO:26) or up to the full sequence of the hinge region of trastuzumab, that contains or has the sequence EPKSCDKTHTCPPCP (corresponding to residues 219-233 of SEQ ID NO:26), or at least 5, 6, 7, 8, 9, 10, or 11 contiguous residues thereof, or residues ESKYGPPCPPCP, set forth as residues 212-223 of SEQ ID NO:29, or a sequence having at least 98% or 99% sequence identity thereto that is a linker;
b) a linker that is or comprises a glycine-serine (GS) linker;
c) a GS linker selected from among (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)ₙ, where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS;
d) a linker that comprises a GS linker and all or a portion of the hinge sequence of trastuzumab, corresponding to residues EPKSCDKTHTCPPCP, set forth as residues 219-233 of SEQ ID NO:26;
e) a linker that comprises a GS linker and comprises the sequence SCDKTH, corresponding to residues 217-222 of SEQ ID NO:31; and
f) a linker that comprises a GS linker and all or a portion of the hinge sequence of nivolumab, corresponding to residues 212-223 of SEQ ID NO:29.

Linker can be selected from among:
i) a GS linker selected from among (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)ₙ, where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS;
ii) a second linker selected from among all or a portion of the hinge sequence of trastuzumab and all or a portion of the hinge sequence of nivolumab; and
iii) a half-life extending moiety that is an IgG Fc.

The constructs can comprise a dAb set forth in any of SEQ ID NOs:54-83, 503-672, 1478 and 1479 or these include the dAbs whose sequence are set forth in any of SEQ ID NOs: 55-84, 86-88, 450, and 495-498, which bind to the same epitope as dAb of SEQ ID NO:59, and variants thereof having at least 95%, 96%, 97%, 98%, or 99% sequence identity thereto, whereby the construct has TNFR1 antagonist activity, and does not have agonist activity.

The constructs comprise modified Fc's where the unmodified Fc is an IgG1 or IgG4 Fc; and where:
the IgG1 Fc is selected from the IgG1 Fc of human IgG1, set forth in SEQ ID NO:10, or the IgG1 Fc of trastuzumab, set forth in SEQ ID NO:27;
the IgG4 Fc is selected from the IgG4 Fc of human IgG4, set forth in SEQ ID NO:16, or the IgG4 Fc of nivolumab, set forth in SEQ ID NO:30; and
optionally, the Fc includes one or more modifications to introduce knobs-into-holes, and/or increase or enhance neonatal Fc receptor (FcRn) recycling, and/or reduce or eliminate immune effector functions.

The modified Fc can be is an IgG Fc that further comprises modification(s) to increase or enhance neonatal Fc receptor (FcRn) recycling. Modifications in the Fc portions include, for example Fc portions selected from among:
a) an Fc that comprises knobs-into-holes modifications, wherein:
   the knob mutation is selected from among one or more of S354C, T366Y, T366W, and T394W by EU numbering; and
   the hole mutation is selected from among one or more of Y349C, T366S, L368A, F405A, Y407T, Y407A, and Y407V by EU numbering;
b) an Fc that comprises modifications to increase or enhance FcRn recycling that is/are selected from among one or more of: T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering;
c) an Fc that comprises modifications to immune effector functions that are selected from among one or more of complement-dependent cytotoxicity (CDC), antibody-dependent cell-mediated cytotoxicity (ADCC) and antibody-dependent cell-mediated phagocytosis (ADCP);
d) a construct that comprises modification(s) in the Fe to reduce or eliminate immune effector functions, wherein the Fc and modifications are selected from among one or more of:
in IgG1: L235E, L234A/L235A, L234E/L235F/P331S, L234F/L235E/P331S, L234A/L235A/P329G, L234A/L235A/G237A/P238S/H268A/A330S/P331S, G236R/L328R, G237A, E318A, D265A, E233P, N297A, N297Q, N297D, N297G, N297G/D265A, A330L, D270A, P329A, P331A, K322A, V264A, and F241A, by EU numbering; and
in IgG4: L235E, F234A/L235A, S228P/L235E, and S228P/F234A/L235A, by EU numbering;
e) an Fc that is an IgG Fc that comprises one or more of the following modifications:
   i) a modification(s) to introduce knobs-into-holes, wherein:
   the knob mutation is selected from among one or more of S354C, T366Y, T366W, and T394W by EU numbering; and
   the hole mutation is selected from among one or more of Y349C, T366S, L368A, F405A, Y407T, Y407A, and Y407V by EU numbering;
   ii) a modification(s) to increase or enhance neonatal Fc receptor (FcRn) recycling, wherein the modification is selected from among one or more of:
   T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering; and
   iii) a modification(s) to increase or enhance immune effector functions, wherein:
   the immune effector functions are selected from among one or more of CDC, ADCC and ADCP; and
   the modification(s) to increase or enhance immune effector functions is selected from among one or more of:
      in IgG1: S239D; 1332E; S239D/I332E; S239D/A330L/I332E; S298A/E333A/K334A; F243L/R292P/Y300L/V305I/P396L; L235V/F243L/R292P/Y300L/P396L; F243L/R292P/Y300L; L234Y/G236W/S298A in the first heavy chain and S239D/A330L/I332E in the second heavy chain; L234Y/L235Q/G236W/S239M/H268D/D270E/S298A in the first heavy chain and D270E/K326D/A330M/K334E in the second heavy chain; A327Q/P329A; D265A/S267A/H268A/D270A/K326A/S337A; T256A/K290A/S298A/E333A/K334A; G236A; G236A/1332E; G236A/S239D/I332E; G236A/S239D/A330L/I332E; introduction of a biantennary glycan at residue N297; introduction of an afucosylated glycan at residue N297; K326W; K326A; E333A; K326A/E333A; K326W/E333 S; K326M/E333 S; K222W/T223W; K222W/T223W/H224W; D221W/K222W; C220D/D221C; C220D/D221C/K222W/T223W; H268F/S324T; S267E; H268F; S324T; S267E/H268F/S324T; G236A/I332E/S267E/H268F/S324T; E345R; and E345R/E430G/S440Y, by EU numbering; and
f) an Fc that is modified to increase binding to the inhibitory Fcγ receptor (FcγR) FcγRIIb.
g) combinations of a)-f). Generally, in the constructs herein, the Fc's include modifications to increase half-life and at least one or two additional modification.

Modifications that increase binding to FcγRIIb are selected from among one or more of S267E, N297A, L328F, L351S, T366R, L368H, P395K, S267E/L328F and L351S/T366R/L368H/P395K, by EU numbering.

In particular examples, the constructs comprises the dAb of SEQ ID NO:59 or a variant thereof having at least 95% sequence identity thereto and binding to the same epitope as SEQ ID NO:59 linked directly or indirectly via a linker to a modified Fc, wherein the Fc is modified to have increased half-life. Exemplary thereof are those designated EN1206-BV and EN1206-MV, wherein the EN1206-MV construct comprises Fc's with knobs into holes. Constructs comprising polypeptides of SEQ ID NOs: 1488-1490 and/or SEQ ID NOs:1505-1507 and variants thereof, with 95% or greater sequence identity, that bind TNRF1 and have anti-TNFR1 antagonist activity and retain the half-life of constructs. Variants generally have at least 95% sequence identity with the recited sequence. Exemplary constructs include a monovalent construct, comprising the dAb-knob chain (SEQ ID NO: 1506) linked to the Fe hole chain (SEQ ID NO:1505), and variants thereof that bind TNRF1 and have anti-TNFR1 antagonist activity and retain the half-life of constructs, wherein the knob and hole Fc portions form an Fc multimer that displays the dAb portion. Also exemplary is a bivalent construct comprising the polypeptide of SEQ ID NO: 1507 and variants thereof that bind TNRF1 and have anti-TNFR1 antagonist activity and retain the half-life of constructs.

Provided are pharmaceutical compositions containing one or a mixture of any of the constructs provided herein in a pharmaceutically acceptable vehicle. The constructs and pharmaceutical compositions are for use for treating a disease, disorder, or condition that is treated by inhibiting TNFR1 or in methods of treating such diseases, disorders, and conditions by administering the composition or construct. Diseases, disorders, and conditions include, for example, a chronic inflammatory, autoimmune, neurodegenerative, demyelinating, or respiratory disease or disorder, or a disease, condition or disorder characterized by overexpression of TNF or deregulated TNFR1 signaling in its etiology. Diseases, disorders, and conditions include, but are not limited to: rheumatoid arthritis (RA), psoriasis, psoriatic arthritis, juvenile idiopathic arthritis (JIA), spondylarthritis, ankylosing spondylitis, Crohn's disease, ulcerative colitis, inflammatory bowel disease (IBD), uveitis, fibrotic diseases, endometriosis, lupus, multiple sclerosis (MS), congestive heart failure, cardiovascular disease, myocardial infarction (MI), atherosclerosis, metabolic diseases, cytokine release syndrome, septic shock, sepsis, acute respiratory distress syndrome (ARDS), severe acute respiratory syndrome (SARS), SARS-CoV-2, influenza, acute and chronic neurodegenerative diseases, demyelinating diseases and disorders, stroke, Alzheimer's disease, Parkinson's disease, Behget's disease, Dupuytren's disease, Tumor Necrosis Factor Receptor-Associated Periodic Syndrome (TRAPS), pancreatitis, type I diabetes, chronic obstructive pulmonary disease (COPD), chronic bronchitis, emphysema, graft rejection, graft versus host disease (GvHD), lung inflammation, pulmonary diseases and conditions, asthma, cystic fibrosis, idiopathic pulmonary fibrosis, acute fulminant viral or bacterial infections, pneumonia, genetically inherited diseases with TNF/TNFR1 as the causative pathologic mediator, periodic fever syndrome, or cancer.

Methods for identifying a construct or TNFR1 inhibitor that is a TNFR1 antagonist, and that is not a TNFR1 agonist are provided. Exemplary of such methods is a methods comprising:
a) identifying a construct that has high affinity binding, such as, for example, nanomolar amount (10⁻⁹ M) or less. specific for TNFR1;
b) screening the constructs of a) using an assay that identifies constructs that exhibit inhibit TNF-stimulated expression of NF-κB and/or expression of inflammatory cytokines; and
c) from among the constructs identified in b), selecting those that lack agonist activity. For example, in such methods, the assays in a) and c) can be THP1 cell assays.

Also provided are constructs that are a tumor necrosis factor receptor 1 (TNFR1) antagonist construct of formula 1:

(TNFR1 inhibitor)ₙ-linkerₚ-(activity modifier)_{q}, wherein:
each of n and q is an integer, n is 1 or 2, and q is 1;
p is 0, 1, 2 or 3;
a TNFR1 inhibitor is a molecule that binds TNFR1 to inhibit (antagonize) TNFR1;
the TNFR1 inhibitor binds to the same epitope as the dAb of SEQ ID NO:59, whereby a construct that is bivalent, where n is 2, does not have agonist activity;
an activity modifier is a moiety that modulates or alters the activity or the pharmacological property of the construct compared to the construct in the absence of the activity modifier and is a modified Fc or human serum albumin (HSA), where the Fc is modified to have increased half-life and reduced immune effector activity compared to the unmodified Fc; and
the linker increases flexibility of the construct, and/or moderates or reduces steric effects of the construct or its interaction with a receptor, and/or increases solubility in aqueous media of the construct.

Also provided are constructs that are tumor necrosis factor receptor 1 (TNFR1) antagonist constructs of formula 1:

(TNFR1 inhibitor)ₙ-linkerₚ-(activity modifier)_{q}, wherein:
each of n and q is an integer, n is 1 or 2, and q is 1;
p is 0, 1, 2 or 3;
a TNFR1 inhibitor is a molecule that binds TNFR1 to inhibit (antagonize) TNFR1;
an activity modifier is a moiety that modulates or alters the activity or the pharmacological property of the construct compared to the construct in the absence of the activity modifier;
a linker increases flexibility of the construct, and/or moderates or reduces steric effects of the construct or its interaction with a receptor, and/or increases solubility of the construct in aqueous medium; the construct is a fusion protein that comprises a TNFR1 inhibitor, wherein the resulting construct, when bivalent, where n is 2, does not have TNRF1 agonist activity;
when n is 2 whereby the construct is bivalent, the TNFR1 inhibitor is selected so that the construct has antagonist activity, but does not have agonist activity;
the activity modifier is a modified Fc that has one or more of mutations to increase serum half-life and comprises modifications for the purpose of silencing the antibody effector function; and, when n=1 and q=2, the Fc has a modification(s) to introduce knobs-into-holes.

The Fc portions of the constructs include those that have one or more modification(s) to increase or enhance neonatal Fc receptor (FcRn) recycling; and modification(s) to reduce or eliminate immune effector functions. TNFR1 inhibitors include, but are not limited to, a TNFR1 inhibitor that comprises an scFv, or a Fab, or that is a nanobody, or a dominant-negative TNF or a TNF mutein, or that is a dAb. The TNFR1 inhibitor can be one that is a selective antagonist comprises an antigen-binding fragment that selectively binds and inhibits TNFR1 signaling but not TNFR2 signaling.

Also provided are construct that further comprise a TNFR2 agonist, such as construct that comprises the formula:

(TNFR1 inhibitor)₁-(activity modifier)ᵣ₁(Linker(L))ₚ-(activity modifier)ᵣ₂-(TNFR2 agonist)_{q}, or

(TNFR1 inhibitor)₁-(activity modifier)ᵣ₁(Linker(L))ₚ-(activity modifier)ᵣ₂-(Treg expander)_{q},
where: n=lor 2, or 3, p=1, 2, or 3, q=0, 1 or 2, and each of r1 and r2 is independently 0, 1, or 2; and the components can be in the order specified or any other order as long as the construct interacts with TNFR1 and TNFR2 to antagonize TNFR1 and agonize TNFR2, or has Treg expander activity, but does not have TNFR1 agonist activity.

Nucleic acid molecules encoding any of the constructs and constituent chains are provided. Also provided are vectors and cells containing the vectors and nucleic acids. Methods for producing the constructs by expressing the chains and constructs in the cells and isolating the resulting chains and constructs are provided. The chains can be produced separately, isolated, and combined into constructs. Alternatively, mixtures of the constructs can be co-expressed in the cells and the constructs of interest isolated, and, if needed, separated. Those of skill in the art are familiar with the variety of methods by which products, such as antibodies are produced.

The claims set forth in the application as filed and as filed in the priority application are incorporated by reference into this Summary.

### BRIEF DESCRIPTION OF THE DRAWINGS

FIG. 1 depicts a plasmid map of the pCBL-1 expression plasmid containing the CMV promoter where TE19080L is the inserted fragment.

FIG. 2 sets forth an exemplary bi-specific construct—with a linker (part of a hinge region) and activity modifier joining two ligands, such as TNFR1 inhibitor (TNFR1 antagonist) and a TNFR2 agonist.

FIGS. 3A-3D depict exemplary PEG-centered multi-specific constructs, which are for presenting/providing two or more moieties that interact with one or more targets, or with one target at a plurality of sites. FIG. 3A depicts an exemplary bivalent construct. One of the circles is, for example, a polypeptide agonist, antagonist or a binding protein, such as an antibody or antigen-binding fragment thereof, or an aptamer (nucleic acid or peptide). The other circle represents polysaccharides or receptor ligands or other moieties that interact with a target of interest. The bivalent nature provides for clustering of targets for receptor activation. In embodiments provided herein, the targets include TNFR1 and TNFR2; and as described throughout the disclosure herein, moieties include TNFR1 inhibitors, such as moieties that inhibit TNFR1 signaling, and TNFR2 agonists or other moieties that are Treg expanders.

FIG. 3B depicts a monovalent single ligand, such as CD3+, to prevent cytokine release syndrome, linked via the PEG moieties to the agonist, antagonist, or binding protein, which is bivalent for receptor clustering. Again, exemplary targets include TNFR1 and/or TNFR2. FIG. 3C depicts a heterobifunctional PEG for crosslinking two different cell targeting agents, or two agents, such as trastuzumab and pertuzumab or portions thereof, that bind to different sites on the same receptor. This construct can be used, for example, to cluster a checkpoint control receptor for either stimulation or inhibition of an immune response, or to crosslink two different receptors to achieve suppression of receptor activity (i.e., CD3 vs CD450, or to deliver two different ligands, such as a stimulatory and a co-stimulatory ligand, to two different receptors on the same cells. FIG. 3D depicts a homobifunctional PEG for clustering identical receptors on the same or different cells, depending upon chain length, or to trap circulating disease target, such as a soluble receptor or ligand, such as TNF. Additionally, in all of these embodiments additional PEG side chain(s), optionally linked to another reactive group or functional group, such as a serum half-life extending moiety, such as HSA, or an FcRn polypeptide, can be included in these constructs. The PEG moieties can be modified or replaced with moieties with similar properties for presentation of the binding moieties.

FIG. 4 depicts additional exemplary configurations and structures of PEG-centered constructs for displaying or providing binding moieties or reactive moieties, such as the TNFR1 inhibitors and/or the TNFR2 agonists as described herein.

FIG. 5 depicts additional exemplary configurations and structures of PEG-centered constructs for displaying or providing binding moieties or reactive moieties, such as the TNFR1 inhibitors and/or the TNFR2 agonists. X and Y can be ligands and reactive moieties.

FIG. 6 shows the effects of the exemplary construct designated Vhh-4 (see Example 4) on gene expression in THP1 cells that were stimulated with TNF-α. These effects were compared to the effects on gene expression by etanercept (Enbrel®) and adalimumab/Humira® for the ability to suppress TNF-induced gene expression of interleukin-6, IL-8 and TNF (three inflammatory cytokines). Controls (four bars on the left side of each panel) show the level of cytokine expression when cells are exposed to the inhibitors in the absence of added TNF. The four bars on the right side of each panel show the level of cytokine expression in the presence of TNF. The first bar on the right side of each panel shows relative TNF-induced gene expression of the respective cytokine (IL-6, IL-8, TNF) in the absence of inhibitor. The next bar, in each of the graphs, shows the relative level of cytokine expression in the presence of Vhh-4, the next bars show results in the in the presence of etanercept/Enbrel®, and adalimumab/Humira®. TNF-induced gene expression of IL-6, IL-8 and TNF was reduced about 10-fold in each case, indicating that Vhh-4 is at least as potent as etanercept/Enbrel® or adalimumab/Humira® (n=3; ±SEM; *p<0.05; **p<0.01; ***p<0.001).

FIGS. 7A and 7B show relative gene expression of TNF-induced inflammatory cytokine release in THP1 cells treated with TNF and different does and combinations of EN 1206, 206, 541(208), and 019. FIG. 7A shows the relative gene expression of CXCL8(IL8) in THP1 cells treated with one of EN-1206, 206, 541(208), or 019 each at 32, 16, 8, or 4 nM or treated with combinations of 206+541, 206+019, and 541+019 each at concentrations of 8 or 4 nM. The 206 peptide shows the greatest reduction in CXCL8(IL8) relative gene expression of all the tested constructs and combinations. FIG. 7B shows the relative gene expression of TNF in THP1 cells treated with one of EN-1206, 206, 541(208), or 019 each at 32, 16, 8, or 4 nM, or treated with combinations of 206+541, 206+019, and 541+019 each at concentrations of 8 or 4 nM. The 206 peptide alone shows the greatest reduction in TNF relative gene expression of all the tested constructs and combinations.

FIGS. 8A and 8B depict TNF inhibitor constructs. FIG. 8A shows the structure of constructs designated EN1000-206 (206), EN1206-MV (monovalent), EN1206-BV (bivalent), and EN1208-BV. EN-1000-206 (206), is a humanized nanobody binding to domain 4 of TNFR1. EN-1000-208 is a humanized nanobody binding to domain 3 of TNFR1. EN-1206-Fc-MV (EN1206-MV or EN1206-OA) is a one-armed antibody with the EN-1000-206 nanobody fused without a linker to the N-terminus a human Fc region of human IgG1a. EN-1206-Fc-BV (EN1206-BV or EN1206) is a bivalent antibody knob-in-hole construct with EN-1000-206 fused with the N-terminus of 2 IgG1a Fc subunits. EN-1208-Fc-BV (EN1208-BV or EN1208) is similar to EN-1206-Fc-BV but with EN-1000-208 fused with two subunits of IgG1a. The Knobs-into-holes mutation of EN1206-MV facilitates heterodimer pairing.

FIG. 8B depicts the constructs EN1206-MV, EN1206-BV, including the knob-in-holds, and EN1208-BV,

FIG. 9 is an illustrative depiction of the target binding assay performed to assess the binding of each of EN1206-MV, EN1206-BV, and EN1208-BV to the analytes hTNFR1 and hTNFR2 using a protein A chip. Also included are the binding curves from the human TNFR1 binding assay for EN1206-MV, EN1206-BV, and EN1208-BV.

FIG. 10 is an illustrative depiction of the target binding assay performed to assess the binding of EN1206-BV and EN1208-BV to the analytes hTNFR1 and hTNFR2 using a CM5 chip. Also included are the binding curves for TNFR1 to each of EN1206-BV and EN1208-BV.

FIGS. 11A and 11B show the binding curves and quantified (table), dose dependent binding of EN1206-mv and EN1208-bv to immobilized soluble human TNFR1. Octet RED96 was used to measure the binding affinity of EN1206mv and EN1208bv. FIG. 11A shows dose-depending binding of EN1206mv to immobilized TNFR1 on HIS1k chip detected within the concentration orange of 0.412 nM to 100 nM. FIG. 11B shows dose dependent binding of EN1208bv to the immobilized TNFR1 on SA chip detected within the concentration range of 3.7 nm to 300 nM.

FIGS. 12A and 12B depict ligand binding to TNFR1 that is blocked by EN1206-MV (EN1206-OA), EN1206-BV (EN1206), and EN1208-BV (EN1208).

FIG. 12A is a diagram depicting ELISA binding assay setup. FIG. 12B binding curves demonstrate that binding of TNFR1 to the immobilized TNF-α (left panel) or lymphotoxin (TNF-β) (right panel) can be blocked by EN1206-MV (circles), EN1206-BV (squares), or EN1208-BV (diamonds) in a dose-dependent manner. EN1206-BV is a potent TNFR1 antagonist.

FIGS. 13A-C show CXCL8(IL8) (FIG. 13A) and TNF-α (FIG. 13B) relative gene expression in THP1 cells treated with 0, 1, 3, or 10 nM of an anti-TNFR1 agent (206 VhH (the dAB), EN1206-MV, EN1206-BV, or EN1208-BV) followed by a 6-hour treatment of TNF-α (100 ng/mL). The RNA samples were collected, and qPCR analysis was performed to analyze gene expression using HPRT as an internal control (N=3, mean±SEM). FIG. 13C shows a comparison of EN-1000-206 Domain Antibody (206dAb) vs.EN-1206-Fc-MV (monovalent) and EN-1206-Fc-BV (bivalent) for Suppression of TNF-induced Expression of CXCL8(IL8) and TNF; the horizontal line (relative gene expression=1) indicates baseline expression vs. the HGPRT.

FIGS. 14A-E are sets of bar graphs that quantify the relative potency of CXCL8(IL8) and TNF inhibition by EN-1000-206dAb vs. EN-1206-Fc-MV, BV. The data shown are the background subtracted ratio of gene expression values from FIG. 13C wherein the potency of EN-1000-206dAb=1. FIG. 14A is the relative potency for EN-1206-Fc-MV suppression of TNF expression. FIG. 14B is the relative potency for EN-1206-Fc-BV suppression of TNF expression. FIG. 14C is the relative potency for EN-1206-Fc-MV suppression of CXCL8(IL8) expression. FIG. 14D is the relative potency for EN-1206-Fc-BV suppression of CXCL8 (IL8) expression. FIG. 14E is a table summarizing the quantified results of bar graphs in FIGS. 14A-D.

FIG. 15 is a graph that quantifies the percentage of cytokine expression, IL8(CXCL8) (left panel), TNF (right panel) following TNF-α treatment as a function of concentration of anti-TNFR1 agent administered (0.05, 0.1, 0.25, 0.5, 1, 3 or 10 nM of etanercept (Enbrel®) (triangles), or EN1206-BV (circles), or EN1206-MV (squares)). The graphs show the quantified results of the qPCR analysis of gene expression using HPRT as an internal control (N=3, mean±SEM).

FIG. 16 is a second graph that quantifies the percentage of cytokine expression, IL8(CXCL8) (left panel), TNF-α (right panel) following TNF-α treatment as a function of concentration of anti-TNFR1 agent administered (0.05, 0.1, 0.25, 0.5, 1, 3 or 10 nM of etanercept (Enbrel®) (light grey), or EN1206-BV (black), or EN1206-MV (grey)). The results demonstrate that monovalent and bivalent forms of EN-1206-Fc are more potent than etanercept in blocking TNF-induced cytokine release.

FIG. 17 depicts a diagram (right drawing) of the binding assay using the Octet Streptavidin (SA) biosensor to immobilize the TNFR1-His-avi tag protein to assess the binding of EN1208-BV (703291) and EN1206-MV (70269_1). The graph shows the results of the epitope binning assay wherein the Streptavidin (SA) biosensor chip was used to immobilize the TNFR1. This was followed by binding of the EN1208bv (the 1^{st} antibody) and EN1206mv (the 2^{nd} antibody), sequentially, to the immobilized TNFR1. Each generated a binding curve typical of non-overlapping epitopes confirming that EN1206/Fc-mv and EN1208/Fc-bv bind to the non-overlapping, separate epitopes.

FIG. 18 is a table, which illustrates the amino acid sequences of the dAbs that bind to human TNFR1 (from Enever et al., Stress selections on domain antibodies: ‘what doesn't kill you makes you stronger’. Protein Eng Des Sel., 28(3):59-66 (2015)). One of the clones studied, designated Dom1h-131-511(highlighted), is a VH dAb that binds to domain three of human TNFR1. As detailed in Enever et al., after stability optimization, top variants were selected and their mutations in CDRs and frameworks were compared. The variants derived from Dom1h-131-511 also included dAbs designated Dom1h-131-202, -203, -204, and -206 (highlighted). Compared to the parent clone Dom-131-511, the optimized variant Dom1h-131-206 (SEQ ID NO:59) showed better binding affinity to TNFR1 (Enever et al., Table II), more resistant to proteases (Enever et al., FIG. 2), improved melting temperature and EC50 (Enever et al., Table III).

FIG. 19 provides sequence alignment between and among Dom1h-131-206 (EN1206) and other dABs to select sequences (in black) that will bind the same epitope in TNFR1. It was found herein that constructs that comprise the -206 dAb, do not exhibit agonist activity. The EN1206 dAb is the VHH that binds to TNFR1 and that is the affinity and stability optimized clone derived from the parental clone DOM1 h-131-511; the underlined portions of sequence in DOM1 h-131-206 (SEQ ID NO:59) comprise the three heavy chain complementarity-determining regions (HCDR) that bind domain 3 of TNFR1 ECD (HCDR1: AHETMV; HCDR2: HIPPDGQDPFYADSVKG; HCDR3: LPKRGPWFDY). The optimization processes generated many other variants as well, see complete list of aligned sequences in FIG. 19. The VHH portion of EN1206 or DOM1 h-131-206 was selected from among other variants. Sequence alignment herein indicates there is only one AA difference between DOM1 h-131-511 CDRs (parental) and DOM1 h-131-206 CDRs (3%). Among the dAbs (SEQ ID NOs: 54-672, at least 36 (in black text) bind to the same epitope based on the combined AA similarity in the three CDRs of each sequence. Any dAbs with sequences having >~95% sequence identity to the CDRs will bind to the same epitope as the -206 dAb. These include the dAbs set forth in any of SEQ ID NOs: 55-84, 86-88, 450, 495-498 bind to the same epitope as EN 1206.

FIG. 20 is a diagram illustrating an assay to show that EN1206 suppresses in vivo TNF-induced cytokine expression.

FIG. 21 are the quantified results of an in vivo assay showing the EN-1206-Fc-MV suppresses in vivo LPS-induced IL-6 and CXCL1. The assay involves a 2 hour Predose with Infliximab, an anti-TNF antibody sold under the brand names Remicade® and Inflectra®) at 10 mg/kg, or EN-1206-Fc-MV at 3 mg/kg and 30 mg/kg; Time 0: 10 ug LPS; Time 5H: Serum Samples are collected and ELISA performed. The experimental results confirm the clinical observation that the product sold as Inflectra® leaves behind ~20% of non-responders. Specific downregulation of TNFR1 by EN-1206-Fc-MV gives a more complete response rate, likely due to TREGS with higher FOXP3 levels, see FIGS. 23A-F.

FIG. 22 is a diagram to illustrate the approach used to assess the role of regulatory T cells.

FIGS. 23A-23F show the scatter plots and bar graphs generated from a FACS analysis that demonstrate that EN-1206-Fc-MV and EN-1206-Fc-BV are superior to etanercept (Enbrel®; TNF Blocker) in preserving the human regulatory T cell population in cultures of human T cells (CD25+FOXP3+T-Cells, n=3, mean±SEM). CD4+T cells were thawed and maintained in the presence of IL2 (10 ng/ml) for 2 days. In separate cultures, 2×10⁵ CD4+T cells were treated with TNF-α (100 ng/ml) alone or combined with 10 nM TNF-α Blocker etanercept (Enbrel®) or 10 nM of either P55-TNFRSF1a inhibitor EN-1206-Fc-BV (bivalent) or EN-1206-Fc-MV (monovalent) in the absence or presence of 1 g/ml anti-CD3 and anti-CD28 monoclonal antibodies (activating reagents to amplify proliferation of T cells) for 24 hrs (FIGS. 23A and 23B), 48 hrs (FIGS. 23C and 23D), and 72 hrs (FIGS. 23E and 23F). Cultures were analyzed by FACS gated for CD25^{hi} FOXP3+ without activation (top panels) or with activation (bottom panels). For the bar-graph representation of the FACS data (FIGS. 23B, 23D, and 23F) statistical significance is represented as: *<p0.05. **<p0.01, ***<p0.001 for differences between the samples. The solid bar under each ‘*’ indicates the samples being compared. Background levels of staining intensity were lower in the non-activated samples, but in each sample the highest intensity of staining for Treg cells was present in samples treated with TNF+IL2 as shown on the FACS diagram, and the bar-graph to the right. The bar-graph shows about 67% reduction in FOXP3 with Enbrel, but only 33% reduction when treated with EN-1206-Fc-MV or -BV.

FIGS. 24A and 24B are graphs of TNF-α binding assay data from US 2014/0112929 A1 for comparison with the data herein. FIG. 24A is a graph showing the results of a TNF-α receptor binding assay (RBA) comparing the effect of a non-competitive TNFR1 binding protein (DOM1 h-574-208) and a competitive TNFR1 binding protein (DOM1 h-131-206) on the ability of a TNF-α to bind TNFR1; compare with FIG. 12B. FIG. 24B is a graph showing the results of a TNF-α function assay showing that both competitive and non-competitive TNFR1 binding proteins are capable of inhibiting TNF-α signal transduction; compare with FIGS. 13A and 13B.

### DETAILED DESCRIPTION

Outline
A. DEFINITIONS
B. OVERVIEW OF CONSTRUCTS AND METHODS
C. TUMOR NECROSIS FACTOR (TNF) AND CHRONIC INFLAMMATORY AND AUTOIMMUNE DISEASES AND DISORDERS
1. Tumor Necrosis Factor (TNF)
2. Tumor Necrosis Factor Receptors (TNFRs)
   a. TNFR1
   b. TNFR2
3. Regulatory T Cells (Tregs) and Their Role in the Autoimmune Microenvironment
4. Autoimmune/Inflammatory Diseases Mediated by or involving TNF
   a. Arthritis
      i. Rheumatoid Arthritis and other types of arthritis
   b. Inflammatory Bowel Disease (IBD) and Uveitis
   c. Fibrotic Diseases
   d. Tumor Necrosis Factor Receptor-Associated Periodic Syndrome (TRAPS)
   e. Other Diseases Mediated by or involving TNF
      i. Neurodegenerative Diseases
         a) Alzheimer's Disease
         b) Parkinson's Disease
         c) Multiple Sclerosis (MS)
      ii. Endometriosis
      iii. Cardiovascular Disease
      iv. Acute Respiratory Distress Syndrome (ARDS)
      v. Severe Acute Respiratory Syndrome (SARS) and COVID-19
D. THERAPIES FOR RHEUMATOID ARTHRITIS AND OTHER CHRONIC INFLAMMATORY AND AUTOIMMUNE DISEASES AND DISORDERS
   1. Conventional Synthetic Disease Modifying Anti-Rheumatic Drugs (csDMARDs)
   2. Anti-TNF Therapies/TNF Blockers
E. THERAPEUTICS FOR TARGETING TNFR1/TNFR2
   1. TNFR1-Selective Antagonists
      a. TNFR1 antagonistic Antibodies
      b. Monovalent TNFR1 antagonistic Antibodies/Antibody Fragments
         i. Fab- and scFv-Based TNFR1 antagonists
         ii. Domain Antibody (dAb)-Based TNFR1 antagonists
         a) Anti-TNFR1 dAb-Anti-Albumin dAb Fusion Constructs
         b) Domain antibody fragments designated GSK1995057 and GSK2862277
         iii. Nanobodies (Nbs)
         iv. Anti-TNFR1 Nanobody-Anti-Albumin Nanobody Fusion Constructs
      c. Dominant-Negative Inhibitors of TNF (DN-TNFs)/TNF Muteins
   2. TNFR2-Selective Agonists
      a. TNFR2 agonistic Antibodies
      b. TNFR2-Selective TNF Muteins and Fusions Thereof
   3. Anti-TNFR2 Antagonistic Antibodies and Small Molecule Inhibitors
F. SELECTIVE TARGETING OF THE TNFR1 AND/OR THE TNFR1 AND TNFR2 AXIS
   1. Selective Blockade of TNFR1 with TNFR1 antagonists
   2. Selective Activation of TNFR2 with TNFR2 agonists
   3. TNFR1 antagonist constructs, TNFR2 agonist constructs; Multi-Specific, Including Bi-Specific, TNFR1 Antagonist and TNFR2 Agonist Constructs
   4. Components of the TNFR1 antagonist constructs
      a. TNFR1 inhibitor moiety (TNFR1 antagonist)
      b. Linkers
         i. Peptide Linkers
         a) Flexible linkers
         b) Rigid linkers
         ii. Chemical Linkers
      c. Fc-Activity modifiers
         i. Modifications to the Fc portions
         a) Knobs-in-Holes
         b) Modifications that Enhance Neonatal Fe Receptor (FcRn) Recycling
         c) Enhancement of or Reduction/Elimination of Fc Immune Effector Functions
         ii. Other Modifications of Fc portions
         iii. Human Serum Albumin
      d. Additional Activity Modifiers—Fusion Proteins that Include Portions or Entire Polypeptides that Increase Serum Half-Life
G. ASSESSING TNFR1 ANTAGONIST AND TNFR1 ANTAGONIST/TNFR2 AGONIST CONSTRUCT ACTIVITY AND EFFICACY
   1. Disease Activity Score (DAS28)
   2. SOMAscan® Proteomic Analysis and other proteomic tools for quantifying analytes
   3. Transcriptome Analysis to Predict Responsiveness to Therapy and to select subjects likely to benefit from treatment
   4. L929 Cytotoxicity Assay
   5. HeLa IL-8 Assay
   6. HUVEC Assay
   7. Quantification and Evaluation of Treg Cell Activity
   8. Evaluation of Binding Properties of the TNFR1 antagonist/TNFR2 Agonist Constructs
   9. Antibody-Dependent Cellular Cytotoxicity (ADCC) and Complement-Dependent Cytotoxicity (CDC) Assays
   10. Disease Models
      a. Collagen-Induced Arthritis (CIA)
      b. Rheumatoid Arthritis Synovial Membrane Mononuclear Cell Cultures
      c. Tg197 Mouse Model of Arthritis
      d. AARE Mouse Model of Arthritis/IBD
      e. Humanized TNF/TNFR2 Mice
H. METHODS OF PRODUCING NUCLEIC ACIDS ENCODING TNFR1 ANTAGONIST CONSTRUCTS AND TNFR1 ANTAGONIST/TNFR2 AGONIST CONSTRUCTS
   1. Isolation or Preparation of Nucleic Acids Encoding TNFR1 Antagonist and TNFR2 Agonist Polypeptides
   2. Generation of Mutant or Modified Nucleic Acids and Encoding Polypeptides
   3. Vectors and Cells
   4. Expression
      a. Prokaryotic Cells
      b. Yeast Cells
      c. Insects and Insect Cells
      d. Mammalian Expression Cells
      e. Plants
   5. Purification
   6. Additional Modifications
      a. PEGylation
      b. Albumination
      c. Purification Tags
   7. Nucleic Acid Molecules and Gene Therapy
I. COMPOSITIONS, FORMULATIONS AND DOSAGES
   1. Formulations
   2. Administration of the TNFR1 Antagonist Constructs, TNFR2 Agonist Constructs, the Multi-specific, such as Bi-Specific, Constructs and Nucleic acids
   3. Administration of Nucleic Acids Encoding Polypeptides (Gene Therapy)
J. THERAPEUTIC USES AND METHODS OF TREATMENT
   1. Treatment of Chronic Inflammatory/Autoimmune Diseases and Disorders
   2. Treatment of Neurodegenerative and Demyelinating Diseases and Disorders
   3. Treatment of Cancer and other Immunosuppressing Diseases, Disorders, and Conditions
   4. Combination Therapies
K. EXAMPLES

### A. Definitions

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change, and particular information on the internet can come and go, but equivalent information can be found by searching the internet. Reference thereto evidences the availability and public dissemination of such information.

As used herein, a construct is a product that contains one more components, generally at least two. The components can be polypeptides, small molecules, aptamers, nucleic acids, and/or other such components as described herein or known to those of skill in the art. Various constructs are described and exemplified herein; the components and variety thereof is apparent from the description herein. Those of skill in the art in view of the description can envision other constructs that are within the disclosure and claims herein. The term construct is employed because the products can include a variety of different types of components.

As used herein, a construct that is a TNFR1 construct or a TNFR2 antagonist construct, is a construct that comprises a TNFR1 inhibitor moiety, which is a moiety that inhibits or reduces a TNFR1 activity, such as signaling.

As used herein, a construct that is a TNFR2 construct or a TNFR2 agonist construct, is a construct that comprises a TNFR2 agonist moiety, which is a moiety that activates or induces an activity of a TNFR2, such as signaling or an activity the results in increased Treg cells.

As used herein, a construct that is a TNFR2 antagonist construct, is a construct that comprises a TNFR2 antagonist.

As used herein, a construct that is a multi-specific construct is a construct that comprises more than one antagonist or agonist or both moieties, such as a construct that contains a TNFR1 inhibitor and a TNFR2 agonist, or a construct that contains two TNFR1 antagonists, such as where each interacts with a different epitope on TNFR1 or each has a different TNFR1 antagonist activity, or two TNFR2 agonists, such as where each interacts with a different TNFR2 epitope, or each has a different TNFR2 agonist activity.

As used herein, “tumor necrosis factor,” “tumor necrosis factor alpha,” “TNF,” “TNF-alpha,” “TNF-α” and “TNFα” are used interchangeably to refer to a pleiotropic proinflammatory cytokine that is a member of the TNF superfamily and is associated with inflammatory and immuno-regulatory activities, including the regulation of tumorigenesis/cancer, host defense against pathogenic infections, apoptosis, autoimmunity, and septic shock. When other members of the TNF superfamily are intended, they will be identified by name. TNF participates in coordination of innate and adaptive immune responses, as well as in organogenesis, particularly of the lymphoid organs. TNF is produced as a homotrimeric membrane-bound protein containing 233 amino acids that can be cleaved by the protease TACE (TNF alpha converting enzyme; also known as ADAM17) to release soluble TNF (solTNF), which contains 157 amino acids; membrane-bound and soluble forms of TNF are biologically active. Homotrimers of TNF bind to and signal through two high-affinity, specific receptors, TNFR1 and TNFR2; membrane-bound TNF primarily activates TNFR2, while soluble TNF primarily activates TNFR1. The uncontrolled or dysregulated production of TNF is associated with several chronic inflammatory and autoimmune diseases and conditions, including, but not limited to, for example, septic shock, rheumatoid arthritis, psoriasis, psoriatic arthritis, ankylosing spondylitis, juvenile idiopathic arthritis, and inflammatory bowel disease (IBD), as well as neurodegenerative and demyelinating diseases and conditions, including, but not limited to, for example, Alzheimer's disease, Parkinson's disease, stroke and multiple sclerosis.

As used herein, a “TNF mutein” or “TNF-α mutein” or “modified TNF polypeptide” refers to a polypeptide that has an amino acid sequence that, for TNF from a particular species, differs from the amino acid sequence of a corresponding wild-type TNF (TNF-α) by one or more amino acids. Generally, such modified TNF polypeptides retain the ability to activate or inhibit TNFR1 and/or TNFR2. Specific mutations in TNF can render the resulting TNF mutein selective for binding to TNFR1 or TNFR2, and can result in TNF muteins with antagonistic or agonistic properties. For example, as described herein, there are TNFR1-selective antagonistic TNF muteins, and TNFR2-selective agonistic TNF muteins.

As used herein, a “dominant-negative inhibitor of TNF” or “DN-TNF” is a TNF mutein with one or more mutations that abrogate binding to and signaling through TNFR1 and/or TNFR2. DN-TNFs selectively inhibit soluble TNF (sTNF or solTNF) by rapidly exchanging subunits with native TNF homotrimers, forming inactive mixed TNF heterotrimers with disrupted receptor binding surfaces, thus preventing interaction with TNF receptors. DN-TNFs leave transmembrane TNF (tmTNF) unaffected, maintaining the protective roles of TNF signaling through TNFR2. Examples of DN-TNFs are TNF mutants containing one or more of the replacements L133Y, S162Q, Y163H, I173T, Y191Q and A221R, with reference to the sequence of amino acids set forth in SEQ ID NO:1 (corresponding to residues L57Y, S86Q, Y87H, I97T, Y115Q, and A145R, with reference to the sequence of solTNF, as set forth in SEQ ID NO:2), which impair binding to TNFRs.

As used herein, a “modification” is in reference to the modification of a sequence of amino acids in a polypeptide, or a sequence of nucleotides in a nucleic acid molecule, and includes deletions, insertions, transpositions, replacements and combinations thereof of amino acids or nucleotides, respectively. Methods of modifying a polypeptide or nucleic acid are routine to those of skill in the art, such as by using recombinant DNA methodologies.

As used herein, “deletion,” when referring to a nucleic acid or polypeptide sequence, refers to the deletion of one or more nucleotides or amino acids compared to a sequence, such as a target polynucleotide or polypeptide, or a native or wild-type sequence.

As used herein, “insertion,” when referring to a nucleic acid or amino acid sequence, describes the inclusion of one or more additional nucleotides or amino acids, within a target, native, wild-type or other related sequence. Thus, a nucleic acid molecule that contains one or more insertions compared to a wild-type sequence, contains one or more additional nucleotides within the linear length of the sequence.

As used herein, “addition,” when referring to a nucleic acid or amino acid sequence, describes the addition of one or more nucleotides or amino acids onto either termini, compared to another sequence.

As used herein, a “substitution” or “replacement” refers to the replacing of one or more nucleotides or amino acids in a native, target, wild-type or other nucleic acid or polypeptide sequence, with an alternative nucleotide or amino acid, without changing the length (as described in numbers of residues) of the molecule. Thus, one or more substitutions in a molecule does not change the number of amino acid residues or nucleotides of the molecule. Amino acid replacements compared to a particular polypeptide can be expressed in terms of the number of the amino acid residue along the length of the polypeptide sequence. For example, a modified polypeptide having a modification in the amino acid at the 100^{th} position of the amino acid sequence that is a substitution/replacement of tyrosine (Tyr; Y) with glutamic acid (Glu; E), can be expressed as Y100E, Tyr100Glu, or 100E. Y100 can be used to indicate that the amino acid at the modified 100^{th} position is a tyrosine. For purposes herein, since modifications are in a heavy chain (HC) or light chain (LC) of an antibody, modifications also can be denoted by reference to HC- or LC- to indicate the chain of the polypeptide.

As used herein, “at a position corresponding to,” or recitation that nucleotides or amino acid positions “correspond to” nucleotides or amino acid positions in a disclosed sequence, such as set forth in the Sequence Listing, refers to nucleotides or amino acid positions identified upon alignment with a referenced sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carrillo et al. (1988) SIAM J. Applied Math 48:1073).

As used herein, alignment of a sequence refers to the use of homology to align two or more sequences of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with a genomic DNA sequence. Related or variant polypeptides or nucleic acid molecules can be aligned by any method known to those of skill in the art. Such methods typically maximize matches, and include methods, such as using manual alignments and by using the numerous alignment programs available (e.g., BLASTP) and others known to those of skill in the art. By aligning the sequences of polypeptides or nucleic acids, one skilled in the art can identify analogous portions or positions, using conserved and identical amino acid residues as guides. Further, one skilled in the art also can employ conserved amino acid or nucleotide residues as guides to find corresponding amino acid or nucleotide residues between and among human and non-human sequences. Corresponding positions also can be based on structural alignments, for example, by using computer simulated alignments of protein structure. In other instances, corresponding regions can be identified. One skilled in the art also can employ conserved amino acid residues as guides to find corresponding amino acid residues between and among human and non-human sequences.

As used herein, recitation that proteins are “compared under the same conditions” means that different proteins are treated identically or substantially identically such that any one or more conditions that can influence the activity or properties of a protein or agent are not varied or not substantially varied between the test agents. For example, when the activity of an antibody is compared to another antibody, any one or more conditions, such as the amount or concentration of the polypeptide; the presence, including amount, of excipients, carriers or other components in a formulation other than the active agent (e.g., antibody); temperature; pH; time of storage; storage vessel; properties of storage (e.g., agitation); and/or other conditions associated with exposure or use, are identical or substantially identical between and among the compared polypeptides/antibodies.

As used herein, an “adverse effect,” or “side effect,” or “adverse event,” or “adverse side effect,” refers to a harmful, deleterious and/or undesired effect associated with administering a therapeutic agent. For example, side effects associated with the administration of an anti-TNF antibody, such as adalimumab (sold, for example, under the trademark Humira®), are known to one of skill in the art, and some are described herein. Such adverse side effects include, for example, serious infections, such as tuberculosis, and other infections caused by viruses, fungi and bacteria, including upper respiratory infections, as well as dermatological and dermal toxicity, such as rash, headaches and nausea. Thus, “adverse effect” or “side effect” refers to a harmful, deleterious and/or undesired effect of administering a therapeutic agent. Side effects or adverse effects are graded on toxicity, and various toxicity scales exist, providing definitions for each grade. Examples of such scales are toxicity scales of the National Cancer Institute Common Toxicity Criteria version 2.0, and the World Health Organization or Common Terminology Criteria for Adverse Events (CTCAE) scale. Assigning grades of severity is within the skill of an experienced physician or other health care professional. The severity of symptoms can be quantified using the NCI Common Terminology Criteria for Adverse Events (CTCAE) grading system. The CTCAE is a descriptive terminology used for Adverse Event (AE) reporting. The grading (severity) scale is provided for each AE term. The CTCAE displays Grades 1 through 5, with clinical descriptions for severity for each adverse event based on the following general guideline: Grade 1 (Mild AE); Grade 2 (Moderate AE); Grade 3 (Severe AE); Grade 4 (Life-threatening or disabling AE); and Grade 5 (Death related to AE/fatal).

As used herein, a “property” of a polypeptide, such as an antibody, refers to any property exhibited by a polypeptide, including, but not limited to, binding specificity, structural configuration or conformation, protein stability, resistance to proteolysis, conformational stability, thermal tolerance, and tolerance to pH conditions. Changes in properties can alter an “activity” of the polypeptide. For example, a change in the binding specificity of the antibody polypeptide can alter the ability to bind an antigen, and/or various binding activities, such as affinity or avidity, or in vivo activities of the polypeptide.

As used herein, an “activity” or a “functional activity” of a polypeptide, such as an antibody, refers to any activity exhibited by the polypeptide. Such activities can be empirically determined. Exemplary activities include, but are not limited to, the ability to interact with a biomolecule, for example, through antigen-binding, DNA binding, ligand binding, or dimerization; and enzymatic activity, for example, kinase activity or proteolytic activity. For an antibody (including antibody fragments), activities include, but are not limited to, the ability to specifically bind a particular antigen, affinity of antigen-binding (e.g., high or low affinity), avidity of antigen-binding (e.g., high or low avidity), on-rate, off-rate, effector functions, such as the ability to promote antigen neutralization or clearance, virus neutralization, and in vivo activities, such as the ability to prevent infection or invasion of a pathogen, or to promote clearance, or to penetrate a particular tissue or fluid or cell in the body. Activity can be assessed in vitro or in vivo using recognized assays, such as ELISA, flow cytometry, surface plasmon resonance or equivalent assays to measure on- or off-rate, immunohistochemistry and immunofluorescence histology and microscopy, cell-based assays, flow cytometry, and binding assays (e.g., panning assays). For example, for an antibody polypeptide, activities can be assessed by measuring binding affinities, avidities, and/or binding coefficients (e.g., for on-/off-rates), and other activities in vitro, or by measuring various effects in vivo, such as immune effects, e.g., antigen clearance; penetration or localization of the antibody into tissues; protection from disease, e.g., infection; serum or other fluid antibody titers; or other assays that are well-known in the art. The results of such assays that indicate that a polypeptide exhibits an activity can be correlated to activity of the polypeptide in vivo, in which in vivo activity can be referred to as therapeutic activity, or biological activity. Activity of a modified polypeptide can be any level of percentage of activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, of activity compared to the unmodified polypeptide. Assays to determine functionality or activity of modified (or variant) antibodies are well-known in the art.

As used herein, “bind,” “bound,” and grammatical variations thereof, refers to the participation of a molecule in any attractive interaction with another molecule, resulting in a stable association in which the two molecules are in close proximity to one another. Binding interactions include, but are not limited to, non-covalent bonds, covalent bonds (such as reversible and irreversible covalent bonds), and includes interactions between molecules, such as, but not limited to, proteins, nucleic acids, carbohydrates, lipids, and small molecules, such as chemical compounds, including drugs. Exemplary bonds are antibody-antigen interactions and receptor-ligand interactions. When an antibody “binds” a particular antigen, “bind” refers to the specific recognition of the antigen by the antibody, through cognate antibody-antigen interaction, at antibody combining sites. Binding also can include the association of multiple chains of a polypeptide, such as antibody chains, which interact through disulfide bonds.

As used herein, “binding activity” refers to characteristics of a molecule, e.g., a polypeptide, relating to whether or not, and how, it binds one or more binding partners. Binding activities include the ability to bind the binding partner(s), the affinity with which it binds to the binding partner (e.g., high affinity), the avidity with which it binds to the binding partner, the strength of the bond with the binding partner, and/or the specificity for binding with the binding partner.

As used herein, “affinity” or “binding affinity” describes the strength of the interaction between two or more molecules, such as binding partners, and typically, the strength of the noncovalent interactions between two binding partners. The affinity of an antibody or antigen-binding fragment thereof for an antigen epitope is the measure of the strength of the total noncovalent interactions between a single antibody combining site and the epitope. Low-affinity antibody-antigen interaction is weak, and the molecules tend to dissociate rapidly, while high affinity antibody-antigen binding is strong and the molecules remain bound for a longer amount of time. Binding affinity can be determined in terms of binding kinetics, such as by measuring rates of association (kₐ or kₒₙ) and/or dissociation (k_{d} or k_{off}), half maximal effective concentration (EC₅₀) values, and/or thermodynamic data (e.g., Gibbs free energy (ΔG), enthalpy (ΔH), entropy (−TΔS), and/or calculating association (Kₐ) or dissociation (K_{d}) constants. EC₅₀, also called the apparent K_{d}, is the concentration (e.g., ng/mL) of antibody, where 50% of the maximal binding is observed to a fixed amount of antigen. Typically, EC₅₀ values are determined from sigmoidal dose-response curves, where the EC₅₀ is the concentration at the inflection point. A high antibody affinity for its substrate correlates with a low EC₅₀ value, and a low affinity corresponds to a high EC₅₀ value. Affinity constants can be determined by standard kinetic methodology for antibody reactions, for example, immunoassays, such as ELISA, followed by curve-fitting analysis.

As used herein, “affinity constant” refers to an association constant (Kₐ) used to measure the affinity of an antibody for an antigen. The higher the affinity constant, the greater the affinity of the antibody for the antigen. Affinity constants are expressed in units of reciprocal molarity (i.e., M⁻¹), and can be calculated from the rate constant for the association-dissociation reaction, as measured by standard kinetic methodology for antibody reactions (e.g., immunoassays, surface plasmon resonance, or other kinetic interaction assays known in the art). The binding affinity of an antibody also can be expressed as a dissociation constant, or K_{d}. The dissociation constant is the reciprocal of the association constant, i.e., K_{d}=1/Kₐ. Hence, an affinity constant also can be represented by the K_{d}. Affinity constants can be determined by standard kinetic methodology for antibody reactions, for example, immunoassays, surface plasmon resonance (SPR) (see, e.g., Rich and Myszka (2000) Curr. Opin. Biotechnol 11:54; Englebienne (1998) Analyst. 123:1599), isothermal titration calorimetry (ITC) or other kinetic interaction assays known in the art (see, e.g., Paul, ed., Fundamental Immunology, 2nd ed., Raven Press, New York, pages 332-336 (1989); see also, U.S. Pat. No. 7,229,619, for a description of exemplary SPR and ITC methods for calculating the binding affinity of antibodies).

Instrumentation and methods for real time detection and monitoring of binding rates are known and are commercially available (e.g., BIAcore 2000, BIAcore AB, Upsala, Sweden and GE Healthcare Life Sciences; Malmqvist (2000) Biochem. Soc. Trans. 27:335).

Methods for calculating affinity are well-known, such as methods for determining EC₅₀ values, or methods for determining association/dissociation constants. For example, in terms of EC₅₀, high binding affinity means that the antibody specifically binds to a target protein with an EC₅₀ that is less than about 10 ng/mL, 9 ng/mL, 8 ng/mL, 7 ng/mL, 6 ng/mL, 5 ng/mL, 3 ng/mL, 2 ng/mL, 1 ng/mL or less. High binding affinity also can be characterized by an equilibrium dissociation constant (K_{d}) of 10⁻⁶ M or lower, such as 10⁻⁷ M, 10⁻⁸ M, 10⁻⁹ M, 10⁻¹⁰ M, 10⁻¹¹ M, or 10⁻¹² M, or lower. In terms of equilibrium association constant (Kₐ), high binding affinity is generally associated with Kₐ values of greater than or equal to about 10⁶ M⁻¹, greater than or equal to about 10⁷ M⁻¹, greater than or equal to about 10⁸ M⁻¹, or greater than or equal to about 10⁹ M⁻¹, 101° M⁻¹, 10¹¹ M⁻¹, or 10¹² M⁻¹. Affinity can be estimated empirically, or affinities can be determined comparatively, e.g., by comparing the affinity of two or more antibodies for a particular antigen, for example, by calculating pairwise ratios of the affinities of the antibodies tested. For example, such affinities can be readily determined using conventional techniques, such as by ELISA; equilibrium dialysis; surface plasmon resonance; by radioimmunoassay using a radiolabeled target antigen; or by another method known to the skilled artisan. The affinity data can be analyzed, for example, by the method of Scatchard et al., (1949) Ann N.Y. Acad. Sci., 51:660, or by curve fitting analysis, for example, using a 4 Parameter Logistic nonlinear regression model using the equation: y=((A−D)/(1+((x/C)\hat{ }B)))+D, where A is the minimum asymptote, B is the slope factor, C is the inflection point (EC₅₀), and D is the maximum asymptote.

As used herein, “antibody avidity” refers to the strength of multiple interactions between a multivalent antibody and its cognate antigen, such as with antibodies containing multiple binding sites associated with an antigen with repeating epitopes or an epitope array. A high avidity antibody has a higher strength of such interactions compared to a low avidity antibody.

As used herein, “specificity for a target,” such as TNFR1, refers to a preference, higher binding affinity, for binding to the target compared to a non-target. Selective binding refers to binding to a target with an affinity, generally, of at least about 10⁷-10⁸ M⁻¹. It also can refer to relative activity in which the affinity of a moiety or molecule for one target molecule is compared to the affinity for another molecule, and if the difference is of a certain magnitude, such as about 10-fold, the moiety or molecule is said to have greater specificity for the first target relative to the second.

As used herein, “specifically binds” or “immunospecifically binds,” with respect to an antibody or antigen-binding fragment thereof, are used interchangeably herein and refer to the ability of the antibody or antigen-binding fragment to form one or more noncovalent bonds with a cognate antigen, by noncovalent interactions between the antibody combining site(s) of the antibody and the antigen. Typically, an antibody that immunospecifically binds (or that specifically binds), for example, to TNFR1, is one that binds to TNFR1 with an affinity constant (Kₐ) of about or 1×10⁷ M⁻¹ or 1×10⁸ M⁻¹ or greater (or a dissociation constant (K_{d}) of 1×10⁻⁷ M or 1×10⁻⁸ M or less). Antibodies or antigen-binding fragments that immunospecifically bind to a particular antigen can be identified, for example, by immunoassays, such as radioimmunoassays (RIA), enzyme-linked immunosorbent assays (ELISAs), surface plasmon resonance (SPR), or other techniques known to those of skill in the art.

As used herein, “steric effects” refer to the effects of the size of atoms or groups on the molecule. Steric effects include, but are not limited to, steric hindrance and van der Waals repulsion. Steric effects are the effects resulting from the fact that atoms occupy space; when atoms are put close to each other, this costs energy, as the electrons near the atoms repel each other.

As used herein, “exhibits at least one activity” or “retains at least one activity” refers to the activity exhibited by an antibody polypeptide, such as a variant antibody or other therapeutic polypeptide, compared to the target or unmodified polypeptide, that does not contain the modification. A modified, or variant, polypeptide that retains an activity of a target polypeptide can exhibit improved activity, decreased activity, or maintain the activity of the unmodified polypeptide. In some instances, a modified, or variant, polypeptide can retain an activity that is increased compared to a target or unmodified polypeptide. In some cases, a modified, or variant, polypeptide can retain an activity that is decreased compared to an unmodified or target polypeptide. Activity of a modified, or variant, polypeptide can be any level of percentage of activity of the unmodified or target polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more activity, compared to the unmodified or target polypeptide. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times, greater than the unmodified or target polypeptide. Assays for retention of an activity depend on the activity to be retained.

Such assays can be performed in vitro or in vivo. Activity can be measured, for example, using assays known in the art and described below for activities, such as, but not limited to, ELISA and panning assays. Activities of a modified, or variant, polypeptide compared to an unmodified or target polypeptide also can be assessed in terms of an in vivo therapeutic or biological activity or result following administration of the polypeptide.

As used herein, the “surface plasmon resonance” refers to an optical phenomenon that allows for the analysis of real-time interactions by detection of alterations in protein concentrations within a biosensor matrix. Commercial systems are available. For example, the BIAcore system (GE Healthcare Life Sciences) is an exemplary commercial system.

As used herein, “antibody” refers to immunoglobulins and immunoglobulin fragments, whether natural, or partially or wholly synthetically, such as recombinantly, produced, including any fragment thereof containing at least a portion of the variable heavy chain and/or variable light chain regions of the immunoglobulin molecule that is sufficient to form an antigen-binding site and, when assembled, to specifically bind an antigen. Hence, an antibody includes any protein having a binding domain that is homologous or substantially homologous to an immunoglobulin antigen-binding domain (antibody combining site). For example, an antibody refers to an antibody that contains two heavy chains (which can be denoted H and H′) and two light chains (which can be denoted L and L′), where each heavy chain can be a full-length immunoglobulin heavy chain or a portion thereof sufficient to form an antigen-binding site (e.g., heavy chains include, but are not limited to, V_{H} chains, V_{H}-C_{H}1 chains, and V_{H}-C_{H}1-C_{H}2-C_{H}3 chains), and each light chain can be a full-length light chain or a portion thereof sufficient to form an antigen-binding site (e.g., light chains include, but are not limited to, V_{L} chains and V_{L}-C_{L} chains). Each heavy chain (H and H′) pairs with one light chain (L and L′, respectively). Typically, antibodies minimally include all or at least a portion of the variable heavy (V_{H}) chain and/or the variable light (V_{L}) chain. An antibody also can include other regions, such as, for example, all or a portion of the constant region, and/or all or a portion (sufficient to provide flexibility) of the hinge region.

For purposes herein, the term “antibody,” unless otherwise specified, includes full-length antibodies and portions thereof, including antibody fragments, such as, for example, anti-TNFR1, antibody fragments. Antibody fragments, include, but are not limited to, for example, Fab fragments, Fab′ fragments, F(ab′)₂ fragments, Fv fragments, disulfide-linked Fvs (dsFv), Fd fragments, Fd′ fragments, single-chain Fvs (scFvs), single-chain Fabs (scFab), hsFv (helix-stabilized Fv), single domain antibodies (dAbs, or sdAbs), minibodies, diabodies, anti-idiotypic (anti-Id) antibodies, nanobodies and camelid antibodies, free light chains, VHH antibodies (or nanobodies), or antigen-binding fragments of any of the above. Antibody fragments also can include combinations of any of the above fragments, such as, for example, tandem scFv, Fab-scFv (HC C-term, or LC C-term), Fab-(scFv)₂ (C-term), scFv-Fab-scFv, Fab-C_{H}2-scFv, scFv fusions (C term, or N term), Fab-fusions (HC C-term, or LC C-term), scFv-scFv-dAb, scFv-dAb-scFv, dAb-scFv-scFv, and tribodies. The term “antibody” includes synthetic antibodies, recombinantly produced antibodies, multi-specific and heteroconjugate antibodies (e.g., bi-, tri- and quad-specific antibodies, diabodies, triabodies and tetrabodies), human antibodies, non-human antibodies, humanized antibodies, chimeric antibodies, and intrabodies. Antibodies provided herein include members of any immunoglobulin class (e.g., IgG, IgM, IgD, IgE, IgA and IgY), any subclass (e.g., IgG1, IgG2, IgG3, IgG4, IgA1 and IgA2) or sub-subclass (e.g., IgG2a and IgG2b).

As used herein, a “form of an antibody” refers to a particular structure of an antibody. Antibodies herein include full-length antibodies and portions thereof, such as, for example, a Fab fragment or other antibody fragment. Thus, a Fab is a particular form of an antibody.

As used herein, reference to a “corresponding form” of an antibody means that, when comparing a property or activity of two antibodies, the property is compared using the same form of the antibody. For example, if it is stated that an antibody has less activity compared to the activity of the corresponding form of a first antibody, that means that a particular form, such as a Fab of that antibody, has less activity compared to the Fab form of the first antibody.

As used herein, a full-length antibody is an antibody having two full-length heavy chains (e.g., V_{H}-C_{H}1-C_{H}2-C_{H}3, or V_{H}-C_{H}1-C_{H}2-C_{H}3-C_{H}4), two full-length light chains (V_{L}-C_{L}), and hinge regions, such as human antibodies produced by antibody secreting B cells, and antibodies with the same domains that are produced synthetically.

As used herein, a “multi-specific construct” refers to a construct, such as an antibody or construct comprising portions of an antibody, that exhibits affinity for more than one target antigen so that it can specifically interact with the targets. Multi-specific constructs herein can have structures similar to full immunoglobulin molecules and include Fc regions, for example IgG Fc regions, and antigen-binding regions, such as portions that specifically bind to TNFR1 or TNFR2.

As used herein, a “bispecific construct” refers to a multi-specific construct that has binding specificity for two different antigens. Bispecific constructs include, for example, monoclonal antibodies or antigen-binding fragments thereof linked to a polypeptide region, such as Fc or modified Fc, that modifies the activity of the construct. For human therapeutics, the constructs are derived from human sources or are derived from a human source or are humanized, and the constructs have binding specificities for at least two different antigens. Bi-specific constructs/molecules provided herein can have binding specificities that are directed to TNFR1, and TNFR2. For example, the bi-specific constructs include a TNFR1 antagonist and a TNFR2 agonist. A bispecific antibody or construct includes antibodies and antigen-binding fragment thereof that includes two separate antigen-binding domains (e.g., two scFvs, or two dAbs, or two Fabs, joined by a linker). The antigen-binding domains can bind to the same antigen or different antigens.

As used herein, “antibody fragment” or “antibody portion” refers to any portion of a full-length antibody that is less than full-length, but contains at least a portion of the variable region(s) of the antibody sufficient to form an antigen-binding site (e.g., one or more complementarity-determining region (CDRs)), and thus, retains the binding specificity and/or an activity of the full-length antibody; antibody fragments include antibody derivatives produced by enzymatic treatment of full-length antibodies, as well as synthetically, e.g., recombinantly, produced derivatives.

Examples of antibody fragments include, but are not limited to, Fab, Fab′, F(ab)₂, single-chain Fvs (scFvs), Fv, dsFv, diabody, triabody, affibody, nanobody, aptamer, dAb, Fd and Fd fragments (see, for example, Methods in Molecular Biology, Vol 207: Recombinant Antibodies for Cancer Therapy Methods and Protocols (2003); Chapter 1; pp. 3-25, Kipriyanov). The fragment can include multiple chains linked together, such as by disulfide bridges, and/or by peptide linkers. An antibody fragment generally contains at least about 50 amino acids, such as at about or at least 100 amino acids, and typically, at least about or at least 110, 120, 150, 170, 180, or 200 amino acids.

As used herein, an “Fv antibody fragment” is composed of one variable heavy domain (V_{H}) and one variable light (V_{L}) domain, linked by noncovalent interactions.

As used herein, a dsFv (disulfide-linked Fv) refers to an Fv with an engineered intermolecular disulfide bond, which stabilizes the V_{H}-V_{L} pair.

As used herein, an “scFv fragment” refers to an antibody fragment that contains a variable light chain (V_{L}) and variable heavy chain (V_{H}), covalently connected by a polypeptide linker in any order. The linker is of a length, such that the two variable domains are bridged without substantial interference. Exemplary linkers are (Gly-Ser)₁ residues with some Glu or Lys residues dispersed throughout to increase solubility.

As used herein, “diabodies” are dimeric scFv; diabodies typically have shorter peptide linkers than scFvs, and preferentially dimerize.

As used herein, “triabodies” are trimeric scFv; they contain three peptide chains, each of which contains one V_{H} domain and one V_{L} domain joined by a short linker (e.g., a linker composed of 1-2 amino acids) to permit intramolecular association of V_{H} and V_{L} domains within the same peptide chain; triabodies typically trimerize.

As used herein, a “Fab fragment” is an antibody fragment that results from digestion of a full-length immunoglobulin with papain, or a fragment having the same structure that is produced synthetically, e.g., by recombinant methods. A Fab fragment contains a light chain (containing a V_{L} and C_{L}), and another chain containing a variable domain of a heavy chain (V_{H}) and one constant region domain of the heavy chain (C_{H}1).

As used herein, a “F(ab′)₂ fragment” is an antibody fragment that results from digestion of an immunoglobulin with pepsin at pH 4.0-4.5, or a fragment having the same structure that is produced synthetically, e.g., by recombinant methods. The F(ab′)₂ fragment essentially contains two Fab fragments, where each heavy chain portion contains an additional few amino acids, such as, for example, all or a portion, sufficient to provide flexibility, of the hinge region, including cysteine residues that form disulfide linkages joining the two fragments.

As used herein, a Fab′ fragment is a fragment containing one half (i.e., one heavy chain and one light chain) of the F(ab′)₂ fragment.

As used herein, an Fd fragment is a fragment of an antibody containing a variable domain (V_{H}) and one constant region domain (C_{H}1) of an antibody heavy chain.

As used herein, an Fd′ fragment is a fragment of an antibody containing one heavy chain portion of a F(ab′)₂ fragment.

As used herein, an Fv′ fragment is a fragment containing only the V_{H} and V_{L} domains of an antibody molecule.

As used herein, hsFv (helix-stabilized Fv) refers to an antibody fragment in which the constant domains normally present in a Fab fragment have been substituted with a heterodimeric coiled-coil domain (see, e.g., Arndt et al. (2001) J. Mol. Biol. 7:312:221-228).

As used herein, a “domain antibody,” “single domain antibody,” “sdAb,” or “dAb,” used interchangeably, refers to a monomeric small antibody fragment that contains a variable domain of the heavy chain (V_{H}) or of the light chain (V_{L}) of an antibody. dAbs are the smallest antigen-binding fragments of antibodies; they are about approximately 11-15 kDa in size (about 100-150 amino acids), which is approximately one-tenth the size of a full monoclonal antibody (mAb). There are three complementarity determining regions (CDRs) on each V_{H} and each V_{L}. Each dAb contains three out of the six CDRs, which are the highly diversified loop regions that bind to the target antigen, from a V_{H}-V_{L} pair in an antibody.

As used herein, a camelid antibody, also referred to as a nanobody or VHHs, lacks a light chain and is composed of two identical heavy chains. They occur naturally in camelids, such as camels and alpacas.

As used herein, a polypeptide “domain” is a part of a polypeptide (a sequence of 3 or more, generally 5, 10, or more, amino acids) that is structurally and/or functionally distinguishable or definable. An exemplary polypeptide domain is a part of the polypeptide that can form an independently folded structure within a polypeptide made up of one or more structural motifs (e.g., combinations of alpha helices and/or beta strands connected by loop regions), and/or that is recognized by a particular functional activity, such as enzymatic activity, dimerization or antigen-binding. A polypeptide can have one or more, typically more than one, distinct domains. For example, the polypeptide can have one or more structural domains and one or more functional domains. A single polypeptide domain can be distinguished based on structure and function. A domain can encompass a contiguous linear sequence of amino acids. Alternatively, a domain can encompass a plurality of non-contiguous amino acid portions, which are non-contiguous along the linear sequence of amino acids of the polypeptide. Typically, a polypeptide contains a plurality of domains. For example, each heavy chain and each light chain of an antibody molecule contains a plurality of immunoglobulin (Ig) domains, each about 110 amino acids in length. Those of skill in the art are familiar with polypeptide domains and can identify them by virtue of structural and/or functional homology with other such domains. For exemplification herein, definitions are provided, but it is understood that it is well within the skill in the art to recognize particular domains by name. If needed, appropriate software can be employed to identify domains.

As used herein, a “functional region” of a polypeptide is a region of the polypeptide that contains at least one functional domain (which imparts a particular function, such as an ability to interact with a biomolecule, for example, through antigen-binding, DNA binding, ligand binding, or dimerization, or by enzymatic activity, for example, kinase activity or proteolytic activity); exemplary functional regions of polypeptides are antibody domains, such as V_{H}, V_{L}, C_{H}, C_{L}, and portions thereof, such as CDRs, including CDR1, CDR2 and CDR3, or antigen-binding portions, such as antibody combining sites.

As used herein, a “structural region” of a polypeptide is a region of the polypeptide that contains at least one structural domain.

As used herein, an “Ig domain” is a domain, recognized as such by those in the art, that is distinguished by a structure, called the Immunoglobulin (Ig) fold, which contains two beta-pleated sheets, each containing anti-parallel beta strands of amino acids connected by loops. The two beta sheets in the Ig fold are sandwiched together by hydrophobic interactions and a conserved intra-chain disulfide bond. Individual immunoglobulin domains within an antibody chain further can be distinguished based on function. For example, a light chain contains one variable region domain (V_{L}) and one constant region domain (C_{L}), while a heavy chain contains one variable region domain (V_{H}) and three or four constant region domains (C_{H}). Each V_{L}, C_{L}, V_{H}, and C_{H} domain is an example of an immunoglobulin domain.

As used herein, a “variable domain,” with reference to an antibody, is a specific immunoglobulin (Ig) domain of an antibody heavy or light chain that contains a sequence of amino acids that varies among different antibodies. Each light chain and each heavy chain has one variable region domain (V_{L} and V_{H}, respectively). The variable domains provide antigen specificity, and thus, are responsible for antigen recognition. Each variable region contains complementarity-determining regions (CDRs) that are part of the antigen-binding site domain and framework regions (FRs).

As used herein, “hypervariable region,” “HV,” “complementarity-determining region,” “CDR” and “antibody CDR” are used interchangeably to refer to one of a plurality of portions within each variable region that together form an antigen-binding site of an antibody. Each variable region domain contains three CDRs, named CDR1, CDR2, and CDR3. The three CDRs are non-contiguous along the linear amino acid sequence, but are proximate in the folded polypeptide. The CDRs are located within the loops that join the parallel strands of the beta sheets of the variable domain.

As used herein, “antigen-binding domain,” “antigen-binding site,” “antigen-binding fragment,” “antigen combining site” and “antibody combining site” are used synonymously to refer to a domain within an antibody that recognizes and physically interacts with the cognate antigen. A native conventional full-length antibody molecule has two conventional antigen-binding sites, each containing portions of a heavy chain variable region and portions of a light chain variable region. A conventional antigen-binding site contains the loops that connect the anti-parallel beta strands within the variable region domains. The antigen combining sites can contain other portions of the variable region domains. Each conventional antigen-binding site contains three hypervariable regions from the heavy chain and three hypervariable regions from the light chain. The hypervariable regions also are called complementarity-determining regions (CDRs).

As used herein, “portion thereof,” with reference to an antibody heavy or light chain, or variable heavy or light chain, refers to a contiguous portion thereof that is sufficient to form an antigen-binding site such that, when assembled into an antibody containing a heavy and light chain, it contains at least 1 or 2, typically 3, 4, 5 or all 6 CDRs of the variable heavy (V_{H}) and variable light (V_{L}) chains sufficient to retain at least a portion of the binding specificity of the corresponding full-length antibody containing all 6 CDRs. Generally, a sufficient antigen-binding site requires the CDR3 of the heavy chain (CDRH3). It typically further requires the CDR3 of the light chain (CDRL3). As described herein, one of skill in the art knows and can identify the CDRs based on Kabat or Chothia numbering (see e.g., Kabat, E. A. et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917).

As used herein, “framework regions” or “FRs” are the domains within the antibody variable region domains that are located within the beta sheets; the FR regions are comparatively more conserved, in terms of their amino acid sequences, than the hypervariable regions. Each variable region contains four framework regions that separate the three hypervariable regions.

As used herein, a “constant region” domain is a domain in an antibody heavy or light chain that contains a sequence of amino acids that is comparatively more conserved among antibodies than the variable region domain. Each light chain has a single light chain constant region (C_{L}) domain, and each heavy chain contains one or more heavy chain constant region (C_{H}) domains, which include, C_{H}1, C_{H}2, C_{H}3 and C_{H}4. Full-length IgA, IgD and IgG isotypes contain C_{H}1, C_{H}2 and C_{H}3 domains and a hinge region, while IgE and IgM contain C_{H}1, C_{H}2, C_{H}3 and C_{H}4 domains. C_{H}1 and C_{L} domains extend the Fab arm of the antibody molecule, thus contributing to the interaction with the antigen and rotation of the antibody arms. Antibody constant regions can serve effector functions, such as, but not limited to, clearance of antigens, pathogens and toxins to which the antibody specifically binds, e.g., through interactions with various cells, biomolecules and tissues.

As used herein, an “antibody hinge region” or “hinge region” refers to a polypeptide region in the heavy chain of the gamma, delta and alpha antibody isotypes, that occurs between the C_{H}1 and C_{H}2 domains, joins the Fab and Fc regions, and has no homology with the other antibody domains. This region is rich in proline residues and provides flexibility to IgG, IgD and IgA antibodies, allowing the two “arms” (each containing one antibody combining site) of the Fab portion to be mobile, assuming various angles with respect to one another as they bind an antigen. This flexibility allows the Fab arms to move in order to align the antibody combining sites to interact with epitopes on cell surfaces or other antigens. Two interchain disulfide bonds within the hinge region stabilize the interaction between the two heavy chains.

In some embodiments provided herein, the synthetically produced antibody fragments contain one or more hinge regions, for example, to promote stability via interactions between two antibody chains. Hinge regions are examples parts of dimerization domains, and, for purposes herein are part of the linkers.

As used herein, a “fragment crystallizable region” or “Fc” or “Fc region” or “Fc domain” refers to a polypeptide containing the constant region of an antibody heavy chain, excluding the first constant region immunoglobulin domain. Fc refers to the last two constant region immunoglobulin domains of IgA, IgD, and IgG (C_{H}2 and C_{H}3, also referred to as Cγ2 and Cγ3), or the last three constant region immunoglobulin domains of IgE and IgM (C_{H}2, C_{H}3 and C_{H}4). Optionally, an Fc domain can include all or part of the flexible hinge region, which is N-terminal to these domains. For IgA and IgM, the Fc can include the J chain. For an exemplary Fc domain of IgG, Fc contains immunoglobulin domains C_{H}2 and C_{H}3, and optionally, all or part of the hinge between C_{H}1 and C_{H}2 (also referred to as Cγ1 and Cγ2). The boundaries of the Fc region can vary, but typically, include at least part of the hinge region. For purposes herein, Fc also includes any allelic or species variant, or any variant or modified form, such as any variant or modified form of Fc that has altered binding to an Fc receptor (FcR) or alters an Fc-mediated effector function. Mutations in the Fc region and their effects are well-documented in the art.

As used herein, “Fc chimera” refers to a chimeric polypeptide in which one or more polypeptides is/are linked, directly or indirectly, to an Fc region or a derivative thereof. Typically, an Fc chimera combines the Fc region of an immunoglobulin with another polypeptide. Derivatives of, or modified Fc polypeptides, are known to those of skill in the art.

As used herein, “Kabat numbering” refers to the index numbering of the IgG1 Kabat antibody (see e.g., Kabat, E. A. et al. (1991) Sequences of Proteins of Immunological Interest, Fifth Edition, U.S. Department of Health and Human Services, NIH Publication No. 91-3242); it permits easy comparison among antibodies, similar to way chymotrypsin numbering permits comparison among proteases. One of skill in the art can identify regions of the constant region using Kabat numbering.

As used herein, “EU numbering” or “EU index” refer to the numbering scheme of the EU antibody described in Edelman et al., (1969) Proc. Natl. Acad. Sci. USA 63:78-85. “EU index as in Kabat” refers to EU index numbering of the human IgG1 Kabat antibody as set forth in Kabat, E. A. et al. (1991) Sequences of Proteins of Immunological Interest, Fifth Edition, U.S. Department of Health and Human Services, NIH Publication No. 91-3242. EU numbering, or EU numbering as in Kabat, are frequently used by those of skill in the art to number amino acid residues of the Fc regions of the light and heavy antibody chains. For example, one of skill in the art can identify regions of the constant region using EU numbering. For example, the C_{L} domain of the Ig kappa light chain corresponds to residues R108-C214 according to Kabat and EU numbering (see, e.g., Table 2 below). The C_{H}1 domain of IgG1 corresponds to residues 118-215 (EU numbering) or 114-223 (Kabat numbering); C_{H}2 corresponds to residues 231-340 (EU numbering) or 244-360 (Kabat numbering); C_{H}3 corresponds to residues 341-447 (EU numbering) or 361-478 (Kabat numbering).

The following tables define the numbering for the IgG1 and IgG4 heavy chain constant domains, and the Ig kappa light constant domain, by EU, Kabat, and sequential numbering. Table 1 shows the IgG1 heavy chain constant domain by EU, Kabat and sequential numbering, where sequential numbering is with respect to the sequence of amino acids set forth in SEQ ID NO:9, and identifies residues within the C_{H}1, C_{H}2 and C_{H}3 domains, as well as the hinge region. Table 2 shows the immunoglobulin (Ig) kappa light chain constant domain by EU, Kabat and sequential numbering, where sequential numbering is with respect to the sequence of amino acids set forth in SEQ ID NO:17. In Table 2, the top row (bold) sets forth the amino acid residue number by sequential numbering (with reference to SEQ ID NO:17); the second row (bold) provides the 1-letter code for the amino acid residue at the position indicated by the number in the top row; the third row (in italics) indicates the corresponding Kabat number according to Kabat numbering; and the fourth row indicates the corresponding EU index number according to EU numbering. Table 3 shows the IgG4 heavy chain constant domain by EU, Kabat and sequential numbering, where sequential numbering is with respect to the sequence of amino acids set forth in SEQ ID NO:15, and identifies residues within the C_{H}1, C_{H}2 and C_{H}3 domains, as well as the hinge region.

TABLE 1
IgG1 Heavy Chain Constant Domain by
EU, Kabat and Sequential Numbering

Residue Numbering

| | EU | | Sequential | IgG1 |
|---|---|---|---|---|
| Domain | Index | Kabat | (SEQ ID NO: 9) | Sequence |
| CH1 | 118 | 114 | 1 | A |
| CH1 | 119 | 115 | 2 | S |
| CH1 | 120 | 116 | 3 | T |
| CH1 | 121 | 117 | 4 | K |
| CH1 | 122 | 118 | 5 | G |
| CH1 | 123 | 119 | 6 | P |
| CH1 | 124 | 120 | 7 | S |
| CH1 | 125 | 121 | 8 | V |
| CH1 | 126 | 122 | 9 | F |
| CH1 | 127 | 123 | 10 | P |
| CH1 | 128 | 124 | 11 | L |
| CH1 | 129 | 125 | 12 | A |
| CH1 | 130 | 126 | 13 | P |
| CH1 | 131 | 127 | 14 | S |
| CH1 | 132 | 128 | 15 | S |
| CH1 | 133 | 129 | 16 | K |
| CH1 | 134 | 130 | 17 | S |
| CH1 | 135 | 133 | 18 | T |
| CH1 | 136 | 134 | 19 | S |
| CH1 | 137 | 135 | 20 | G |
| CH1 | 138 | 136 | 21 | G |
| CH1 | 139 | 137 | 22 | T |
| CH1 | 140 | 138 | 23 | A |
| CH1 | 141 | 139 | 24 | A |
| CH1 | 142 | 140 | 25 | L |
| CH1 | 143 | 141 | 26 | G |
| CH1 | 144 | 142 | 27 | C |
| CH1 | 145 | 143 | 28 | L |
| CH1 | 146 | 144 | 29 | V |
| CH1 | 147 | 145 | 30 | K |
| CH1 | 148 | 146 | 31 | D |
| CH1 | 149 | 147 | 32 | Y |
| CH1 | 150 | 148 | 33 | F |
| CH1 | 151 | 149 | 34 | P |
| CH1 | 152 | 150 | 35 | E |
| CH1 | 153 | 151 | 36 | P |
| CH1 | 154 | 152 | 37 | V |
| CH1 | 155 | 153 | 38 | T |
| CH1 | 156 | 154 | 39 | V |
| CH1 | 157 | 156 | 40 | S |
| CH1 | 158 | 157 | 41 | W |
| CH1 | 159 | 162 | 42 | N |
| CH1 | 160 | 163 | 43 | S |
| CH1 | 161 | 164 | 44 | G |
| CH1 | 162 | 165 | 45 | A |
| CH1 | 163 | 166 | 46 | L |
| CH1 | 164 | 167 | 47 | T |
| CH1 | 165 | 168 | 48 | S |
| CH1 | 166 | 169 | 49 | G |
| CH1 | 167 | 171 | 50 | V |
| CH1 | 168 | 172 | 51 | H |
| CH1 | 169 | 173 | 52 | T |
| CH1 | 170 | 174 | 53 | F |
| CH1 | 171 | 175 | 54 | P |
| CH1 | 172 | 176 | 55 | A |
| CH1 | 173 | 177 | 56 | V |
| CH1 | 174 | 178 | 57 | L |
| CH1 | 175 | 179 | 58 | Q |
| CH1 | 176 | 180 | 59 | S |
| CH1 | 177 | 182 | 60 | S |
| CH1 | 178 | 183 | 61 | G |
| CH1 | 179 | 184 | 62 | L |
| CH1 | 180 | 185 | 63 | Y |
| CH1 | 181 | 186 | 64 | S |
| CH1 | 182 | 187 | 65 | L |
| CH1 | 183 | 188 | 66 | S |
| CH1 | 184 | 189 | 67 | S |
| CH1 | 185 | 190 | 68 | V |
| CH1 | 186 | 191 | 69 | V |
| CH1 | 187 | 192 | 70 | T |
| CH1 | 188 | 193 | 71 | V |
| CH1 | 189 | 194 | 72 | P |
| CH1 | 190 | 195 | 73 | S |
| CH1 | 191 | 196 | 74 | S |
| CH1 | 192 | 197 | 75 | S |
| CH1 | 193 | 198 | 76 | L |
| CH1 | 194 | 199 | 77 | G |
| CH1 | 195 | 200 | 78 | T |
| CH1 | 196 | 203 | 79 | Q |
| CH1 | 197 | 205 | 80 | T |
| CH1 | 198 | 206 | 81 | Y |
| CH1 | 199 | 207 | 82 | I |
| CH1 | 200 | 208 | 83 | C |
| CH1 | 201 | 209 | 84 | N |
| CH1 | 202 | 210 | 85 | V |
| CH1 | 203 | 211 | 86 | N |
| CH1 | 204 | 212 | 87 | H |
| CH1 | 205 | 213 | 88 | K |
| CH1 | 206 | 214 | 89 | P |
| CH1 | 207 | 215 | 90 | S |
| CH1 | 208 | 216 | 91 | N |
| CH1 | 209 | 217 | 92 | T |
| CH1 | 210 | 218 | 93 | K |
| CH1 | 211 | 219 | 94 | V |
| CH1 | 212 | 220 | 95 | D |
| CH1 | 213 | 221 | 96 | K |
| CH1 | 214 | 222 | 97 | K |
| CH1 | 215 | 223 | 98 | V |
| Hinge | 216 | 226 | 99 | E |
| Hinge | 217 | 227 | 100 | P |
| Hinge | 218 | 228 | 101 | K |
| Hinge | 219 | 232 | 102 | S |
| Hinge | 220 | 233 | 103 | C |
| Hinge | 221 | 234 | 104 | D |
| Hinge | 222 | 235 | 105 | K |
| Hinge | 223 | 236 | 106 | T |
| Hinge | 224 | 237 | 107 | H |
| Hinge | 225 | 238 | 108 | T |
| Hinge | 226 | 239 | 109 | C |
| Hinge | 227 | 240 | 110 | P |
| Hinge | 228 | 241 | 111 | P |
| Hinge | 229 | 242 | 112 | C |
| Hinge | 230 | 243 | 113 | P |
| CH2 | 231 | 244 | 114 | A |
| CH2 | 232 | 245 | 115 | P |
| CH2 | 233 | 246 | 116 | E |
| CH2 | 234 | 247 | 117 | L |
| CH2 | 235 | 248 | 118 | L |
| CH2 | 236 | 249 | 119 | G |
| CH2 | 237 | 250 | 120 | G |
| CH2 | 238 | 251 | 121 | P |
| CH2 | 239 | 252 | 122 | S |
| CH2 | 240 | 253 | 123 | V |
| CH2 | 241 | 254 | 124 | F |
| CH2 | 242 | 255 | 125 | L |
| CH2 | 243 | 256 | 126 | F |
| CH2 | 244 | 257 | 127 | P |
| CH2 | 245 | 258 | 128 | P |
| CH2 | 246 | 259 | 129 | K |
| CH2 | 247 | 260 | 130 | P |
| CH2 | 248 | 261 | 131 | K |
| CH2 | 249 | 262 | 132 | D |
| CH2 | 250 | 263 | 133 | T |
| CH2 | 251 | 264 | 134 | L |
| CH2 | 252 | 265 | 135 | M |
| CH2 | 253 | 266 | 136 | I |
| CH2 | 254 | 267 | 137 | S |
| CH2 | 255 | 268 | 138 | R |
| CH2 | 256 | 269 | 139 | T |
| CH2 | 257 | 270 | 140 | P |
| CH2 | 258 | 271 | 141 | E |
| CH2 | 259 | 272 | 142 | V |
| CH2 | 260 | 273 | 143 | T |
| CH2 | 261 | 274 | 144 | C |
| CH2 | 262 | 275 | 145 | V |
| CH2 | 263 | 276 | 146 | V |
| CH2 | 264 | 277 | 147 | V |
| CH2 | 265 | 278 | 148 | D |
| CH2 | 266 | 279 | 149 | V |
| CH2 | 267 | 280 | 150 | S |
| CH2 | 268 | 281 | 151 | H |
| CH2 | 269 | 282 | 152 | E |
| CH2 | 270 | 283 | 153 | D |
| CH2 | 271 | 284 | 154 | P |
| CH2 | 272 | 285 | 155 | E |
| CH2 | 273 | 286 | 156 | V |
| CH2 | 274 | 287 | 157 | K |
| CH2 | 275 | 288 | 158 | F |
| CH2 | 276 | 289 | 159 | N |
| CH2 | 277 | 290 | 160 | W |
| CH2 | 278 | 291 | 161 | Y |
| CH2 | 279 | 292 | 162 | V |
| CH2 | 280 | 295 | 163 | D |
| CH2 | 281 | 296 | 164 | G |
| CH2 | 282 | 299 | 165 | V |
| CH2 | 283 | 300 | 166 | E |
| CH2 | 284 | 301 | 167 | V |
| CH2 | 285 | 302 | 168 | H |
| CH2 | 286 | 303 | 169 | N |
| CH2 | 287 | 304 | 170 | A |
| CH2 | 288 | 305 | 171 | K |
| CH2 | 289 | 306 | 172 | T |
| CH2 | 290 | 307 | 173 | K |
| CH2 | 291 | 308 | 174 | P |
| CH2 | 292 | 309 | 175 | R |
| CH2 | 293 | 310 | 176 | E |
| CH2 | 294 | 311 | 177 | E |
| CH2 | 295 | 312 | 178 | Q |
| CH2 | 296 | 313 | 179 | Y |
| CH2 | 297 | 314 | 180 | N |
| CH2 | 298 | 317 | 181 | S |
| CH2 | 299 | 318 | 182 | T |
| CH2 | 300 | 319 | 183 | Y |
| CH2 | 301 | 320 | 184 | R |
| CH2 | 302 | 321 | 185 | V |
| CH2 | 303 | 322 | 186 | V |
| CH2 | 304 | 323 | 187 | S |
| CH2 | 305 | 324 | 188 | V |
| CH2 | 306 | 325 | 189 | L |
| CH2 | 307 | 326 | 190 | T |
| CH2 | 308 | 327 | 191 | V |
| CH2 | 309 | 328 | 192 | L |
| CH2 | 310 | 329 | 193 | H |
| CH2 | 311 | 330 | 194 | Q |
| CH2 | 312 | 331 | 195 | D |
| CH2 | 313 | 332 | 196 | W |
| CH2 | 314 | 333 | 197 | L |
| CH2 | 315 | 334 | 198 | N |
| CH2 | 316 | 335 | 199 | G |
| CH2 | 317 | 336 | 200 | K |
| CH2 | 318 | 337 | 201 | E |
| CH2 | 319 | 338 | 202 | Y |
| CH2 | 320 | 339 | 203 | K |
| CH2 | 321 | 340 | 204 | C |
| CH2 | 322 | 341 | 205 | K |
| CH2 | 323 | 342 | 206 | V |
| CH2 | 324 | 343 | 207 | S |
| CH2 | 325 | 344 | 208 | N |
| CH2 | 326 | 345 | 209 | K |
| CH2 | 327 | 346 | 210 | A |
| CH2 | 328 | 347 | 211 | L |
| CH2 | 329 | 348 | 212 | P |
| CH2 | 330 | 349 | 213 | A |
| CH2 | 331 | 350 | 214 | P |
| CH2 | 332 | 351 | 215 | I |
| CH2 | 333 | 352 | 216 | E |
| CH2 | 334 | 353 | 217 | K |
| CH2 | 335 | 354 | 218 | T |
| CH2 | 336 | 355 | 219 | I |
| CH2 | 337 | 357 | 220 | S |
| CH2 | 338 | 358 | 221 | K |
| CH2 | 339 | 359 | 222 | A |
| CH2 | 340 | 360 | 223 | K |
| CH3 | 341 | 361 | 224 | G |
| CH3 | 342 | 363 | 225 | Q |
| CH3 | 343 | 364 | 226 | P |
| CH3 | 344 | 365 | 227 | R |
| CH3 | 345 | 366 | 228 | E |
| CH3 | 346 | 367 | 229 | P |
| CH3 | 347 | 368 | 230 | Q |
| CH3 | 348 | 369 | 231 | V |
| CH3 | 349 | 370 | 232 | Y |
| CH3 | 350 | 371 | 233 | T |
| CH3 | 351 | 372 | 234 | L |
| CH3 | 352 | 373 | 235 | P |
| CH3 | 353 | 374 | 236 | P |
| CH3 | 354 | 375 | 237 | S |
| CH3 | 355 | 376 | 238 | R |
| CH3 | 356 | 377 | 239 | D |
| CH3 | 357 | 378 | 240 | E |
| CH3 | 358 | 381 | 241 | L |
| CH3 | 359 | 382 | 242 | T |
| CH3 | 360 | 383 | 243 | K |
| CH3 | 361 | 384 | 244 | N |
| CH3 | 362 | 385 | 245 | Q |
| CH3 | 363 | 386 | 246 | V |
| CH3 | 364 | 387 | 247 | S |
| CH3 | 365 | 388 | 248 | L |
| CH3 | 366 | 389 | 249 | T |
| CH3 | 367 | 390 | 250 | C |
| CH3 | 368 | 391 | 251 | L |
| CH3 | 369 | 392 | 252 | V |
| CH3 | 370 | 393 | 253 | K |
| CH3 | 371 | 394 | 254 | G |
| CH3 | 372 | 395 | 255 | F |
| CH3 | 373 | 396 | 256 | Y |
| CH3 | 374 | 397 | 257 | P |
| CH3 | 375 | 398 | 258 | S |
| CH3 | 376 | 399 | 259 | D |
| CH3 | 377 | 400 | 260 | I |
| CH3 | 378 | 401 | 261 | A |
| CH3 | 379 | 402 | 262 | V |
| CH3 | 380 | 405 | 263 | E |
| CH3 | 381 | 406 | 264 | W |
| CH3 | 382 | 407 | 265 | E |
| CH3 | 383 | 408 | 266 | S |
| CH3 | 384 | 410 | 267 | N |
| CH3 | 385 | 411 | 268 | G |
| CH3 | 386 | 414 | 269 | Q |
| CH3 | 387 | 415 | 270 | P |
| CH3 | 388 | 416 | 271 | E |
| CH3 | 389 | 417 | 272 | N |
| CH3 | 390 | 418 | 273 | N |
| CH3 | 391 | 419 | 274 | Y |
| CH3 | 392 | 420 | 275 | K |
| CH3 | 393 | 421 | 276 | T |
| CH3 | 394 | 422 | 277 | T |
| CH3 | 395 | 423 | 278 | P |
| CH3 | 396 | 424 | 279 | P |
| CH3 | 397 | 425 | 280 | V |
| CH3 | 398 | 426 | 281 | L |
| CH3 | 399 | 427 | 282 | D |
| CH3 | 400 | 428 | 283 | S |
| CH3 | 401 | 430 | 284 | D |
| CH3 | 402 | 433 | 285 | G |
| CH3 | 403 | 434 | 286 | S |
| CH3 | 404 | 435 | 287 | F |
| CH3 | 405 | 436 | 288 | F |
| CH3 | 406 | 437 | 289 | L |
| CH3 | 407 | 438 | 290 | Y |
| CH3 | 408 | 439 | 291 | S |
| CH3 | 409 | 440 | 292 | K |
| CH3 | 410 | 441 | 293 | L |
| CH3 | 411 | 442 | 294 | T |
| CH3 | 412 | 443 | 295 | V |
| CH3 | 413 | 444 | 296 | D |
| CH3 | 414 | 445 | 297 | K |
| CH3 | 415 | 446 | 298 | S |
| CH3 | 416 | 447 | 299 | R |
| CH3 | 417 | 448 | 300 | W |
| CH3 | 418 | 449 | 301 | Q |
| CH3 | 419 | 450 | 302 | Q |
| CH3 | 420 | 451 | 303 | G |
| CH3 | 421 | 452 | 304 | N |
| CH3 | 422 | 453 | 305 | V |
| CH3 | 423 | 454 | 306 | F |
| CH3 | 424 | 455 | 307 | S |
| CH3 | 425 | 456 | 308 | C |
| CH3 | 426 | 457 | 309 | S |
| CH3 | 427 | 458 | 310 | V |
| CH3 | 428 | 459 | 311 | M |
| CH3 | 429 | 460 | 312 | H |
| CH3 | 430 | 461 | 313 | E |
| CH3 | 431 | 462 | 314 | A |
| CH3 | 432 | 463 | 315 | L |
| CH3 | 433 | 464 | 316 | H |
| CH3 | 434 | 465 | 317 | N |
| CH3 | 435 | 466 | 318 | H |
| CH3 | 436 | 467 | 319 | Y |
| CH3 | 437 | 468 | 320 | T |
| CH3 | 438 | 469 | 321 | Q |
| CH3 | 439 | 470 | 322 | K |
| CH3 | 440 | 471 | 323 | S |
| CH3 | 441 | 472 | 324 | L |
| CH3 | 442 | 473 | 325 | S |
| CH3 | 443 | 474 | 326 | L |
| CH3 | 444 | 475 | 327 | S |
| CH3 | 445 | 476 | 328 | P |
| CH3 | 446 | 477 | 329 | G |
| CH3 | 447 | 478 | 330 | K |

TABLE 2
Kabat and EU Numbering of Ig Kappa Light Chain Constant Domain

| 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|
| R | T | V | A | A | P | S | V | F | I | F | P | P | S | D |
| 108 | 109 | 110 | 111 | 112 | 113 | 114 | 115 | 116 | 117 | 118 | 119 | 120 | 121 | 122 |
| 108 | 109 | 110 | 111 | 112 | 113 | 114 | 115 | 116 | 117 | 118 | 119 | 120 | 121 | 122 |
| 16 | 17 | 18 | 19 | 20 | 21 | 22 | 23 | 24 | 25 | 26 | 27 | 28 | 29 | 30 |
| E | Q | L | K | S | G | T | A | S | V | V | C | L | L | N |
| 123 | 124 | 125 | 126 | 127 | 128 | 129 | 130 | 131 | 132 | 133 | 134 | 135 | 136 | 137 |
| 123 | 124 | 125 | 126 | 127 | 128 | 129 | 130 | 131 | 132 | 133 | 134 | 135 | 136 | 137 |
| 31 | 32 | 33 | 34 | 35 | 36 | 37 | 38 | 39 | 40 | 41 | 42 | 43 | 44 | 45 |
| N | F | Y | P | R | E | A | K | V | Q | W | K | V | D | N |
| 138 | 139 | 140 | 141 | 142 | 143 | 144 | 145 | 146 | 147 | 148 | 149 | 150 | 151 | 152 |
| 138 | 139 | 140 | 141 | 142 | 143 | 144 | 145 | 146 | 147 | 148 | 149 | 150 | 151 | 152 |
| 46 | 47 | 48 | 49 | 50 | 51 | 52 | 53 | 54 | 55 | 56 | 57 | 58 | 59 | 60 |
| A | L | Q | S | G | N | S | Q | E | S | V | T | E | Q | D |
| 153 | 154 | 155 | 156 | 157 | 158 | 159 | 160 | 161 | 162 | 163 | 164 | 165 | 166 | 167 |
| 153 | 154 | 155 | 156 | 157 | 158 | 159 | 160 | 161 | 162 | 163 | 164 | 165 | 166 | 167 |
| 61 | 62 | 63 | 64 | 65 | 66 | 67 | 68 | 69 | 70 | 71 | 72 | 73 | 74 | 75 |
| S | K | D | S | T | Y | S | L | S | S | T | L | T | L | S |
| 168 | 169 | 170 | 171 | 172 | 173 | 174 | 175 | 176 | 177 | 178 | 179 | 180 | 181 | 182 |
| 168 | 169 | 170 | 171 | 172 | 173 | 174 | 175 | 176 | 177 | 178 | 179 | 180 | 181 | 182 |
| 76 | 77 | 78 | 79 | 80 | 81 | 82 | 83 | 84 | 85 | 86 | 87 | 88 | 89 | 90 |
| K | A | D | Y | E | K | H | K | V | Y | A | C | E | V | T |
| 183 | 184 | 185 | 186 | 187 | 188 | 189 | 190 | 191 | 192 | 193 | 194 | 195 | 196 | 197 |
| 183 | 184 | 185 | 186 | 187 | 188 | 189 | 190 | 191 | 192 | 193 | 194 | 195 | 196 | 197 |
| 91 | 92 | 93 | 94 | 95 | 96 | 97 | 98 | 99 | 100 | 101 | 102 | 103 | 104 | 105 |
| H | Q | G | L | S | S | P | V | T | K | S | F | N | R | G |
| 198 | 199 | 200 | 201 | 202 | 203 | 204 | 205 | 206 | 207 | 208 | 209 | 210 | 211 | 212 |
| 198 | 199 | 200 | 201 | 202 | 203 | 204 | 205 | 206 | 207 | 208 | 209 | 210 | 211 | 212 |

| 106 | 107 |
|---|---|
| E | C |
| 213 | 214 |
| 213 | 214 |

TABLE 3
IgG4 Heavy Chain Constant Domain by
EU, Kabat and Sequential Numbering

Residue Numbering

| | EU | | Sequential | IgG4 |
|---|---|---|---|---|
| Domain | Index | Kabat | (SEQ ID NO: 15) | Sequence |
| CH1 | 118 | 114 | 1 | A |
| CH1 | 119 | 115 | 2 | S |
| CH1 | 120 | 116 | 3 | T |
| CH1 | 121 | 117 | 4 | K |
| CH1 | 122 | 118 | 5 | G |
| CH1 | 123 | 119 | 6 | P |
| CH1 | 124 | 120 | 7 | S |
| CH1 | 125 | 121 | 8 | V |
| CH1 | 126 | 122 | 9 | F |
| CH1 | 127 | 123 | 10 | P |
| CH1 | 128 | 124 | 11 | L |
| CH1 | 129 | 125 | 12 | A |
| CH1 | 130 | 126 | 13 | P |
| CH1 | 131 | 127 | 14 | C |
| CH1 | 132 | 128 | 15 | S |
| CH1 | 133 | 129 | 16 | R |
| CH1 | 134 | 130 | 17 | S |
| CH1 | 135 | 133 | 18 | T |
| CH1 | 136 | 134 | 19 | S |
| CH1 | 137 | 135 | 20 | E |
| CH1 | 138 | 136 | 21 | S |
| CH1 | 139 | 137 | 22 | T |
| CH1 | 140 | 138 | 23 | A |
| CH1 | 141 | 139 | 24 | A |
| CH1 | 142 | 140 | 25 | L |
| CH1 | 143 | 141 | 26 | G |
| CH1 | 144 | 142 | 27 | C |
| CH1 | 145 | 143 | 28 | L |
| CH1 | 146 | 144 | 29 | V |
| CH1 | 147 | 145 | 30 | K |
| CH1 | 148 | 146 | 31 | D |
| CH1 | 149 | 147 | 32 | Y |
| CH1 | 150 | 148 | 33 | F |
| CH1 | 151 | 149 | 34 | P |
| CH1 | 152 | 150 | 35 | E |
| CH1 | 153 | 151 | 36 | P |
| CH1 | 154 | 152 | 37 | V |
| CH1 | 155 | 153 | 38 | T |
| CH1 | 156 | 154 | 39 | V |
| CH1 | 157 | 156 | 40 | S |
| CH1 | 158 | 157 | 41 | W |
| CH1 | 159 | 162 | 42 | N |
| CH1 | 160 | 163 | 43 | S |
| CH1 | 161 | 164 | 44 | G |
| CH1 | 162 | 165 | 45 | A |
| CH1 | 163 | 166 | 46 | L |
| CH1 | 164 | 167 | 47 | T |
| CH1 | 165 | 168 | 48 | S |
| CH1 | 166 | 169 | 49 | G |
| CH1 | 167 | 171 | 50 | V |
| CH1 | 168 | 172 | 51 | H |
| CH1 | 169 | 173 | 52 | T |
| CH1 | 170 | 174 | 53 | F |
| CH1 | 171 | 175 | 54 | P |
| CH1 | 172 | 176 | 55 | A |
| CH1 | 173 | 177 | 56 | V |
| CH1 | 174 | 178 | 57 | L |
| CH1 | 175 | 179 | 58 | Q |
| CH1 | 176 | 180 | 59 | S |
| CH1 | 177 | 182 | 60 | S |
| CH1 | 178 | 183 | 61 | G |
| CH1 | 179 | 184 | 62 | L |
| CH1 | 180 | 185 | 63 | Y |
| CH1 | 181 | 186 | 64 | S |
| CH1 | 182 | 187 | 65 | L |
| CH1 | 183 | 188 | 66 | S |
| CH1 | 184 | 189 | 67 | S |
| CH1 | 185 | 190 | 68 | V |
| CH1 | 186 | 191 | 69 | V |
| CH1 | 187 | 192 | 70 | T |
| CH1 | 188 | 193 | 71 | V |
| CH1 | 189 | 194 | 72 | P |
| CH1 | 190 | 195 | 73 | S |
| CH1 | 191 | 196 | 74 | S |
| CH1 | 192 | 197 | 75 | S |
| CH1 | 193 | 198 | 76 | L |
| CH1 | 194 | 199 | 77 | G |
| CH1 | 195 | 200 | 78 | T |
| CH1 | 196 | 203 | 79 | K |
| CH1 | 197 | 205 | 80 | T |
| CH1 | 198 | 206 | 81 | Y |
| CH1 | 199 | 207 | 82 | T |
| CH1 | 200 | 208 | 83 | C |
| CH1 | 201 | 209 | 84 | N |
| CH1 | 202 | 210 | 85 | V |
| CH1 | 203 | 211 | 86 | D |
| CH1 | 204 | 212 | 87 | H |
| CH1 | 205 | 213 | 88 | K |
| CH1 | 206 | 214 | 89 | P |
| CH1 | 207 | 215 | 90 | S |
| CH1 | 208 | 216 | 91 | N |
| CH1 | 209 | 217 | 92 | T |
| CH1 | 210 | 218 | 93 | K |
| CH1 | 211 | 219 | 94 | V |
| CH1 | 212 | 220 | 95 | D |
| CH1 | 213 | 221 | 96 | K |
| CH1 | 214 | 222 | 97 | R |
| CH1 | 215 | 223 | 98 | V |
| Hinge | 216 | 226 | 99 | E |
| Hinge | 217 | 227 | 100 | S |
| Hinge | 218 | 228 | 101 | K |
| Hinge | 219 | 229 | 102 | Y |
| Hinge | 220 | 230 | 103 | G |
| Hinge | 224 | 237 | 104 | P |
| Hinge | 225 | 238 | 105 | P |
| Hinge | 226 | 239 | 106 | C |
| Hinge | 227 | 240 | 107 | P |
| Hinge | 228 | 241 | 108 | S |
| Hinge | 229 | 242 | 109 | C |
| Hinge | 230 | 243 | 110 | P |
| CH2 | 231 | 244 | 111 | A |
| CH2 | 232 | 245 | 112 | P |
| CH2 | 233 | 246 | 113 | E |
| CH2 | 234 | 247 | 114 | F |
| CH2 | 235 | 248 | 115 | L |
| CH2 | 236 | 249 | 116 | G |
| CH2 | 237 | 250 | 117 | G |
| CH2 | 238 | 251 | 118 | P |
| CH2 | 239 | 252 | 119 | S |
| CH2 | 240 | 253 | 120 | V |
| CH2 | 241 | 254 | 121 | F |
| CH2 | 242 | 255 | 122 | L |
| CH2 | 243 | 256 | 123 | F |
| CH2 | 244 | 257 | 124 | P |
| CH2 | 245 | 258 | 125 | P |
| CH2 | 246 | 259 | 126 | K |
| CH2 | 247 | 260 | 127 | P |
| CH2 | 248 | 261 | 128 | K |
| CH2 | 249 | 262 | 129 | D |
| CH2 | 250 | 263 | 130 | T |
| CH2 | 251 | 264 | 131 | L |
| CH2 | 252 | 265 | 132 | M |
| CH2 | 253 | 266 | 133 | I |
| CH2 | 254 | 267 | 134 | S |
| CH2 | 255 | 268 | 135 | R |
| CH2 | 256 | 269 | 136 | T |
| CH2 | 257 | 270 | 137 | P |
| CH2 | 258 | 271 | 138 | E |
| CH2 | 259 | 272 | 139 | V |
| CH2 | 260 | 273 | 140 | T |
| CH2 | 261 | 274 | 141 | C |
| CH2 | 262 | 275 | 142 | V |
| CH2 | 263 | 276 | 143 | V |
| CH2 | 264 | 277 | 144 | V |
| CH2 | 265 | 278 | 145 | D |
| CH2 | 266 | 279 | 146 | V |
| CH2 | 267 | 280 | 147 | S |
| CH2 | 268 | 281 | 148 | Q |
| CH2 | 269 | 282 | 149 | E |
| CH2 | 270 | 283 | 150 | D |
| CH2 | 271 | 284 | 151 | P |
| CH2 | 272 | 285 | 152 | E |
| CH2 | 273 | 286 | 153 | V |
| CH2 | 274 | 287 | 154 | Q |
| CH2 | 275 | 288 | 155 | F |
| CH2 | 276 | 289 | 156 | N |
| CH2 | 277 | 290 | 157 | W |
| CH2 | 278 | 291 | 158 | Y |
| CH2 | 279 | 292 | 159 | V |
| CH2 | 280 | 295 | 160 | D |
| CH2 | 281 | 296 | 161 | G |
| CH2 | 282 | 299 | 162 | V |
| CH2 | 283 | 300 | 163 | E |
| CH2 | 284 | 301 | 164 | V |
| CH2 | 285 | 302 | 165 | H |
| CH2 | 286 | 303 | 166 | N |
| CH2 | 287 | 304 | 167 | A |
| CH2 | 288 | 305 | 168 | K |
| CH2 | 289 | 306 | 169 | T |
| CH2 | 290 | 307 | 170 | K |
| CH2 | 291 | 308 | 171 | P |
| CH2 | 292 | 309 | 172 | R |
| CH2 | 293 | 310 | 173 | E |
| CH2 | 294 | 311 | 174 | E |
| CH2 | 295 | 312 | 175 | Q |
| CH2 | 296 | 313 | 176 | F |
| CH2 | 297 | 314 | 177 | N |
| CH2 | 298 | 317 | 178 | S |
| CH2 | 299 | 318 | 179 | T |
| CH2 | 300 | 319 | 180 | Y |
| CH2 | 301 | 320 | 181 | R |
| CH2 | 302 | 321 | 182 | V |
| CH2 | 303 | 322 | 183 | V |
| CH2 | 304 | 323 | 184 | S |
| CH2 | 305 | 324 | 185 | V |
| CH2 | 306 | 325 | 186 | L |
| CH2 | 307 | 326 | 187 | T |
| CH2 | 308 | 327 | 188 | V |
| CH2 | 309 | 328 | 189 | L |
| CH2 | 310 | 329 | 190 | H |
| CH2 | 311 | 330 | 191 | Q |
| CH2 | 312 | 331 | 192 | D |
| CH2 | 313 | 332 | 193 | W |
| CH2 | 314 | 333 | 194 | L |
| CH2 | 315 | 334 | 195 | N |
| CH2 | 316 | 335 | 196 | G |
| CH2 | 317 | 336 | 197 | K |
| CH2 | 318 | 337 | 198 | E |
| CH2 | 319 | 338 | 199 | Y |
| CH2 | 320 | 339 | 200 | K |
| CH2 | 321 | 340 | 201 | C |
| CH2 | 322 | 341 | 202 | K |
| CH2 | 323 | 342 | 203 | V |
| CH2 | 324 | 343 | 204 | S |
| CH2 | 325 | 344 | 205 | N |
| CH2 | 326 | 345 | 206 | K |
| CH2 | 327 | 346 | 207 | G |
| CH2 | 328 | 347 | 208 | L |
| CH2 | 329 | 348 | 209 | P |
| CH2 | 330 | 349 | 210 | S |
| CH2 | 331 | 350 | 211 | S |
| CH2 | 332 | 351 | 212 | I |
| CH2 | 333 | 352 | 213 | E |
| CH2 | 334 | 353 | 214 | K |
| CH2 | 335 | 354 | 215 | T |
| CH2 | 336 | 355 | 216 | I |
| CH2 | 337 | 357 | 217 | S |
| CH2 | 338 | 358 | 218 | K |
| CH2 | 339 | 359 | 219 | A |
| CH2 | 340 | 360 | 220 | K |
| CH3 | 341 | 361 | 221 | G |
| CH3 | 342 | 363 | 222 | Q |
| CH3 | 343 | 364 | 223 | P |
| CH3 | 344 | 365 | 224 | R |
| CH3 | 345 | 366 | 225 | E |
| CH3 | 346 | 367 | 226 | P |
| CH3 | 347 | 368 | 227 | Q |
| CH3 | 348 | 369 | 228 | V |
| CH3 | 349 | 370 | 229 | Y |
| CH3 | 350 | 371 | 230 | T |
| CH3 | 351 | 372 | 231 | L |
| CH3 | 352 | 373 | 232 | P |
| CH3 | 353 | 374 | 233 | P |
| CH3 | 354 | 375 | 234 | S |
| CH3 | 355 | 376 | 235 | Q |
| CH3 | 356 | 377 | 236 | E |
| CH3 | 357 | 378 | 237 | E |
| CH3 | 358 | 381 | 238 | M |
| CH3 | 359 | 382 | 239 | T |
| CH3 | 360 | 383 | 240 | K |
| CH3 | 361 | 384 | 241 | N |
| CH3 | 362 | 385 | 242 | Q |
| CH3 | 363 | 386 | 243 | V |
| CH3 | 364 | 387 | 244 | S |
| CH3 | 365 | 388 | 245 | L |
| CH3 | 366 | 389 | 246 | T |
| CH3 | 367 | 390 | 247 | C |
| CH3 | 368 | 391 | 248 | L |
| CH3 | 369 | 392 | 249 | V |
| CH3 | 370 | 393 | 250 | K |
| CH3 | 371 | 394 | 251 | G |
| CH3 | 372 | 395 | 252 | F |
| CH3 | 373 | 396 | 253 | Y |
| CH3 | 374 | 397 | 254 | P |
| CH3 | 375 | 398 | 255 | S |
| CH3 | 376 | 399 | 256 | D |
| CH3 | 377 | 400 | 257 | I |
| CH3 | 378 | 401 | 258 | A |
| CH3 | 379 | 402 | 259 | V |
| CH3 | 380 | 405 | 260 | E |
| CH3 | 381 | 406 | 261 | W |
| CH3 | 382 | 407 | 262 | E |
| CH3 | 383 | 408 | 263 | S |
| CH3 | 384 | 410 | 264 | N |
| CH3 | 385 | 411 | 265 | G |
| CH3 | 386 | 414 | 266 | Q |
| CH3 | 387 | 415 | 267 | P |
| CH3 | 388 | 416 | 268 | E |
| CH3 | 389 | 417 | 269 | N |
| CH3 | 390 | 418 | 270 | N |
| CH3 | 391 | 419 | 271 | Y |
| CH3 | 392 | 420 | 272 | K |
| CH3 | 393 | 421 | 273 | T |
| CH3 | 394 | 422 | 274 | T |
| CH3 | 395 | 423 | 275 | P |
| CH3 | 396 | 424 | 276 | P |
| CH3 | 397 | 425 | 277 | V |
| CH3 | 398 | 426 | 278 | L |
| CH3 | 399 | 427 | 279 | D |
| CH3 | 400 | 428 | 280 | S |
| CH3 | 401 | 430 | 281 | D |
| CH3 | 402 | 433 | 282 | G |
| CH3 | 403 | 434 | 283 | S |
| CH3 | 404 | 435 | 284 | F |
| CH3 | 405 | 436 | 285 | F |
| CH3 | 406 | 437 | 286 | L |
| CH3 | 407 | 438 | 287 | Y |
| CH3 | 408 | 439 | 288 | S |
| CH3 | 409 | 440 | 289 | R |
| CH3 | 410 | 441 | 290 | L |
| CH3 | 411 | 442 | 291 | T |
| CH3 | 412 | 443 | 292 | V |
| CH3 | 413 | 444 | 293 | D |
| CH3 | 414 | 445 | 294 | K |
| CH3 | 415 | 446 | 295 | S |
| CH3 | 416 | 447 | 296 | R |
| CH3 | 417 | 448 | 297 | W |
| CH3 | 418 | 449 | 298 | Q |
| CH3 | 419 | 450 | 299 | E |
| CH3 | 420 | 451 | 300 | G |
| CH3 | 421 | 452 | 301 | N |
| CH3 | 422 | 453 | 302 | V |
| CH3 | 423 | 454 | 303 | F |
| CH3 | 424 | 455 | 304 | S |
| CH3 | 425 | 456 | 305 | C |
| CH3 | 426 | 457 | 306 | S |
| CH3 | 427 | 458 | 307 | V |
| CH3 | 428 | 459 | 308 | M |
| CH3 | 429 | 460 | 309 | H |
| CH3 | 430 | 461 | 310 | E |
| CH3 | 431 | 462 | 311 | A |
| CH3 | 432 | 463 | 312 | L |
| CH3 | 433 | 464 | 313 | H |
| CH3 | 434 | 465 | 314 | N |
| CH3 | 435 | 466 | 315 | H |
| CH3 | 436 | 467 | 316 | Y |
| CH3 | 437 | 468 | 317 | T |
| CH3 | 438 | 469 | 318 | Q |
| CH3 | 439 | 470 | 319 | K |
| CH3 | 440 | 471 | 320 | S |
| CH3 | 441 | 472 | 321 | L |
| CH3 | 442 | 473 | 322 | S |
| CH3 | 443 | 474 | 323 | L |
| CH3 | 444 | 475 | 324 | S |
| CH3 | 445 | 476 | 325 | L |
| CH3 | 446 | 477 | 326 | G |
| CH3 | 447 | 478 | 327 | K |

As used herein, the phrase “derived from,” when referring to antibody fragments derived from another antibody, such as a monoclonal antibody, refers to the engineering of antibody fragments (e.g., Fab, F(ab′), F(ab′)₂, single-chain Fv (scFv), Fv, dsFv, dAb, diabody, Fd and Fd′ fragments) that retain the binding specificity of the original antibody. Such fragments can be derived by a variety of methods known in the art, including, but not limited to, enzymatic cleavage, chemical crosslinking, recombinant means, or combinations thereof. Generally, the derived antibody fragment shares the identical, or substantially identical, heavy chain variable region (V_{H}) and light chain variable region (V_{L}) of the parent antibody, such that the antibody fragment and the parent antibody bind the same epitope.

As used herein, a “parent antibody” or “source antibody” refers to an antibody from which an antibody fragment (e.g., Fab, F(ab′), F(ab)₂, single-chain Fv (scFv), Fv, dsFv, dAb, diabody, Fd and Fd′ fragments) is derived.

As used herein, the term “epitope” refers to any antigenic determinant on an antigen or protein, to which the paratope of an antibody can bind. Epitopic determinants typically contain chemically active surface groupings of molecules, such as amino acids or sugar side chains, and typically have specific three-dimensional structural characteristics, as well as specific charge characteristics.

As used herein, “humanized antibodies” and human therapeutics refer to antibodies and other protein therapeutics that are modified to include “human” sequences of amino acids, so that administration to a human does not provoke an immune response. A humanized antibody, for example, typically contains complementarity determining regions (CDRs or hypervariable loops) derived from a non-human species immunoglobulin, and the remainder of the antibody molecule derived mainly from a human immunoglobulin. Methods for humanizing proteins, including antibodies, and producing them are well known and readily available to those of skill in the art. For example, DNA encoding a monoclonal antibody can be altered by recombinant DNA techniques to encode an antibody in which the amino acid composition of the non-variable regions is based on human antibodies. Methods for identifying such regions are known, including computer programs, which are designed for identifying the variable and non-variable regions of immunoglobulins. Hence, in general, the humanized antibody contains substantially all of at least one, and typically two, variable domains, in which all or substantially all of the hypervariable loops (e.g., CDRs) correspond to those of a non-human immunoglobulin, and all or substantially all of the framework regions (FRs) are those of a human immunoglobulin sequence. The humanized antibody, optionally, also contains at least a portion of an immunoglobulin constant region (Fc), typically that of a human immunoglobulin.

As used herein, a “multimerization domain” refers to a sequence of amino acids that promotes stable interaction of a polypeptide molecule with one or more additional polypeptide molecules, each containing a complementary multimerization domain, which can be the same or a different multimerization domain, to form a stable multimer with the first domain. Generally, a polypeptide is joined directly or indirectly to the multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3 or IgG4 subtypes, IgA, IgE, IgD and IgM, and modified forms thereof.

As used herein, “dimerization domains” are multimerization domains that facilitate interaction between two polypeptide sequences (such as, but not limited to, antibody chains). Dimerization domains include, but are not limited to, an amino acid sequence containing a cysteine residue that facilitates the formation of a disulfide bond between two polypeptide sequences, such as all or a part of a full-length antibody hinge region, or one or more dimerization sequences, which are sequences of amino acids known to promote interaction between polypeptides (e.g., leucine zippers, GCN4 zippers).

As used herein, a “chimeric polypeptide” refers to a polypeptide that contains portions from at least two different polypeptides or from two non-contiguous portions of a single polypeptide. Thus, a chimeric polypeptide generally includes a sequence of amino acid residues from all or a part of one polypeptide, and a sequence of amino acids from all or a part of another different polypeptide. The two portions can be linked directly or indirectly and can be linked via peptide bonds, other covalent bonds, or other non-covalent interactions of sufficient strength to maintain the integrity of a substantial portion of the chimeric polypeptide under equilibrium conditions and physiologic conditions, such as in isotonic pH 7 buffered saline.

As used herein, a “fusion protein” is a polypeptide engineered to contain sequences of amino acids corresponding to two distinct polypeptides, which are joined together, such as by expressing the fusion protein from a vector containing two nucleic acids, encoding the two polypeptides, in close proximity, e.g., adjacent, to one another along the length of the vector. Accordingly, a fusion protein refers to a chimeric protein containing two, or portions from two, or more proteins or peptides that are linked directly or indirectly via peptide bonds. The two molecules can be adjacent in the construct, or can be separated by a linker, or spacer polypeptide.

As used herein, a “linker,” “linker unit,” or “link,” refers to a peptide or chemical moiety containing a chain of atoms that covalently attaches an antibody or antigen-binding fragment thereof to another therapeutic moiety or another antibody or fragment thereof. Linkers are included, for example, to increase flexibility, modify steric effects, including steric hindrance, and increase solubility in aqueous medium.

As used herein, a “linker peptide” or “spacer peptide” refers to short sequences of amino acids that join two polypeptide sequences (or nucleic acids encoding such as an amino acid sequence). “Peptide linker” refers to the short sequence of amino acids joining the two polypeptide sequences. Exemplary of polypeptide linkers are linkers joining a peptide transduction domain to an antibody, or linkers joining two antibody chains in a synthetic antibody fragment, such as an scFv fragment. Linkers are well-known, and any known linkers can be used in the provided methods. Exemplary polypeptide linkers include (Gly-Ser)ₙ amino acid sequences, with some Glu or Lys residues dispersed throughout to increase solubility. Other exemplary linkers are described herein; any of these and other known linkers can be used with the polypeptides, antibodies, and other products and methods provided herein.

As used herein, a “tag” or an “epitope tag” refers to a sequence of amino acids, typically added to the N- or C-terminus of a polypeptide, such as an antibody and an antibody fragment/construct, provided herein. The inclusion of tags fused to a polypeptide can facilitate polypeptide purification and/or detection. Typically, a tag or tag polypeptide refers to a polypeptide that has enough residues to provide an epitope recognized by an antibody, or that can serve for detection or purification, yet is short enough such that it does not interfere with activity of the polypeptide to which it is linked. The tag polypeptide typically is sufficiently unique so that an antibody that specifically binds thereto does not substantially cross-react with epitopes in the polypeptide to which it is linked. Suitable tag polypeptides generally have at least 5 or 6 amino acid residues, and usually between about 8-50 amino acid residues, typically between 9-30 residues. The tags can be linked to one or more chimeric polypeptides in a multimer and permit detection of the multimer or its recovery from a sample or mixture. Such tags are well-known and can be readily synthesized and designed.

Exemplary tag polypeptides include those used for affinity purification and include, for example, FLAG tags; His tags; the influenza hemagglutinin (HA) tag polypeptide and its antibody 12CA5 (see, e.g., Field et al. (1988) Mol. Cell. Biol. 8:2159-2165); the c-myc tag and the 8F9, 3C7, 6E10, G4, B7 and 9E10 antibodies thereto (see, e.g., Evan et al. (1985) Molecular and Cellular Biology 5:3610-3616); and the Herpes Simplex virus glycoprotein D (gD) tag and its antibody (see, e.g., Paborsky et al. (1990) Protein Engineering 3:547-553). An antibody used to detect an epitope-tagged antibody is typically referred to herein as a secondary antibody.

As used herein, a “label” or “detectable moiety” is a detectable marker (e.g., a fluorescent molecule, chemiluminescent molecule, bioluminescent molecule, contrast agent (e.g., a metal), radionuclide, chromophore, detectable peptide, or an enzyme that catalyzes the formation of a detectable product) that can be attached or linked directly or indirectly to a molecule (e.g., an antibody or antigen-binding fragment thereof, such as an anti-TNFR1 antibody or antigen-binding fragment thereof provided herein), or associated therewith, and can be detected in vivo and/or in vitro. The detection method can be any method known in the art, including known in vivo and/or in vitro methods of detection (e.g., imaging by visual inspection, magnetic resonance (MR) spectroscopy, ultrasound signal, X-ray, gamma ray spectroscopy (e.g., positron emission tomography (PET) scanning, single-photon emission computed tomography (SPECT)), fluorescence spectroscopy, or absorption). Indirect detection refers to measurement of a physical phenomenon, such as energy or particle emission or absorption, of an atom, molecule or composition that binds directly or indirectly to the detectable moiety (e.g., detection of a labeled secondary antibody or antigen-binding fragment thereof that binds to a primary antibody (e.g., an anti-TNFR antibody or antigen-binding fragment thereof provided herein)).

As used herein, “nucleic acid” refers to at least two linked nucleotides or nucleotide derivatives, including a deoxyribonucleic acid (DNA) and a ribonucleic acid (RNA), joined together, typically by phosphodiester linkages. Also included in the term “nucleic acid” are analogs of nucleic acids, such as peptide nucleic acid (PNA), phosphorothioate DNA, and other such analogs and derivatives or combinations thereof. Nucleic acids also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (i.e., peptide nucleic acid). The term also includes, as equivalents, derivatives, variants and analogs of either RNA or DNA made from nucleotide analogs, single (sense or antisense) and double-stranded nucleic acids. Deoxyribonucleotides include deoxyadenosine, deoxycytidine, deoxyguanosine and deoxythymidine. For RNA, the uracil base is uridine.

As used herein, an “isolated nucleic acid molecule” is one which is separated from other nucleic acid molecules which are present in the natural source of the nucleic acid molecule. An “isolated” nucleic acid molecule, such as a cDNA molecule, can be substantially free of other cellular material, or culture medium, when produced by recombinant techniques, or substantially free of chemical precursors or other chemicals, when chemically synthesized. Exemplary isolated nucleic acid molecules provided herein include isolated nucleic acid molecules encoding an antibody or antigen-binding fragments provided.

As used herein, “operably linked,” with reference to nucleic acid sequences, regions, elements or domains, means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to nucleic acid encoding a second polypeptide, and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, “synthetic,” with reference to, for example, a synthetic nucleic acid molecule or a synthetic gene or a synthetic peptide, refers to a nucleic acid molecule or gene or polypeptide molecule that is produced by recombinant methods and/or by chemical synthesis methods.

As used herein, the residues of naturally occurring a-amino acids are the residues of those 20 α-amino acids found in nature which are incorporated into a protein by the specific recognition of the charged tRNA molecule with its cognate mRNA codon in humans.

As used herein, “polypeptide” refers to two or more amino acids covalently joined. The terms “polypeptide” and “protein” are used interchangeably herein.

As used herein, a “peptide” refers to a polypeptide that is from 2 to about or 40 amino acids in length.

As used herein, an “amino acid” is an organic compound containing an amino group and a carboxylic acid group. A polypeptide contains two or more amino acids.

For purposes herein, amino acids in the polypeptides, such as antibodies, provided include the twenty naturally-occurring amino acids (Table 4), non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations (see, Table 4). The nucleotides, which occur in the various nucleic acid molecules and fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968), and adopted at 37 C.F.R. §§ 1.821-1.822, abbreviations for amino acid residues are shown in Table 4:

TABLE 4
Table of Correspondence

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glutamic Acid and/or Glutamine |
| W | Trp | Tryptophan |
| R | Arg | Arginine |
| D | Asp | Aspartic acid |
| N | Asn | Asparagine |
| B | Asx | Aspartic Acid and/or Asparagine |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence (Table 4), modified, non-natural and unusual amino acids. Furthermore, a dash at the beginning or end of an amino acid residue sequence indicates a peptide bond to a further sequence of one or more amino acid residues, or to an amino-terminal group, such as NH₂, or to a carboxyl-terminal group, such as COOH. In a peptide or protein, suitable conservative substitutions of amino acids are known to those of skill in the art and generally can be made without altering a biological activity of a resulting molecule. Those of skill in the art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. Co., p. 224).

Such substitutions can be made in accordance with the exemplary substitutions set forth in Table 5 as follows:

TABLE 5
Exemplary Conservative Amino Acid Substitutions

| Original Residue | Conservative Substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, “naturally occurring amino acids” refer to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-stereoisomers of amino acids. Exemplary non-natural amino acids are known to those of skill in the art, and include, but are not limited to, 2-Aminoadipic acid (Aad), 3-Aminoadipic acid (bAad), β-alanine/β-Amino-propionic acid (Bala), 2-Aminobutyric acid (Abu), 4-Aminobutyric acid/piperidinic acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethylglycine (EtGly), N-Ethylasparagine (EtAsn), Hydroxylysine (Hyl), allo-Hydroxylysine (Ahyl), 3-Hydroxyproline (3Hyp), 4-Hydroxyproline (4Hyp), Isodesmosine (Ide), allo-Isoleucine (Aile), N-Methylglycine, sarcosine (MeGly), N-Methylisoleucine (MeIle), 6-N-Methyllysine (MeLys), N-Methylvaline (MeVal), Norvaline (Nva), Norleucine (Nle), and Ornithine (Orn).

As used herein, a “DNA construct” is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a “DNA segment” is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term “polynucleotide” means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules, it is used to denote overall length and is understood to be equivalent to the term base pairs. It will be recognized by those skilled in the art that the two strands of a double-stranded polynucleotide can differ slightly in length and that the ends thereof can be staggered; thus, all nucleotides within a double-stranded polynucleotide molecule cannot be paired. Such unpaired ends will, in general, not exceed 20 nucleotides in length.

As used herein, production by recombinant means by using recombinant DNA methods refers to the use of the well-known methods of molecular biology for expressing proteins encoded by cloned DNA.

As used herein, “expression” refers to the process by which polypeptides are produced by transcription and translation of polynucleotides. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay, and Bradford protein assay.

As used herein, a “host cell” is a cell that is used to receive, maintain, reproduce and/or amplify a vector. A host cell also can be used to express the polypeptide encoded by the vector. The nucleic acid in the vector is replicated when the host cell divides, thereby amplifying the nucleic acids.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide, such as a modified anti-TNFR1 antibody. The vector is used to introduce the nucleic acid encoding the polypeptide into the host cell for amplification of the nucleic acid, or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well-known to those of skill in the art. A vector also includes “virus vectors” or “viral vectors.” Viral vectors are engineered viruses that are operatively linked to exogenous genes to transfer (as vehicles or shuttles) the exogenous genes into cells.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well-known to those of skill in the art and include those that are replicable in eukaryotic cells and/or prokaryotic cells, and those that remain episomal, or those which integrate into the host cell genome.

As used herein, “primary sequence” refers to the sequence of amino acid residues in a polypeptide or the sequence of nucleotides in a nucleic acid molecule.

As used herein, “sequence identity” refers to the number of identical or similar amino acids or nucleotide bases in a comparison between a test and a reference polypeptide or polynucleotide. Sequence identity can be determined by sequence alignment of nucleic acid or protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids or nucleotides inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps, as the number of identical positions/length of the total aligned sequence×100.

As used herein, a “global alignment” is an alignment that aligns two sequences from beginning to end, aligning each letter in each sequence only once. An alignment is produced, regardless of whether or not there is similarity or identity between the sequences. For example, 50% sequence identity based on “global alignment” means that in an alignment of the full sequence of two compared sequences, each of 100 nucleotides in length, 50% of the residues are the same. It is understood that global alignment also can be used in determining sequence identity even when the length of the aligned sequences is not the same. The differences in the terminal ends of the sequences are taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman et al. (1970) J. Mol. Biol. 48:443). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2:482 (1981)). For example, 50% sequence identity based on “local alignment” means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non-identities) and the weighted comparison matrix of Gribskov et al. Nucl. Acids Res. 14:6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences, or any two polypeptides have amino acid sequences, that are at least 80%, 85%, 90%, 95%, 96%, 97%, 98% or 99% “identical,” or other similar variations reciting a percent identity, can be determined using known computer algorithms based on local or global alignment (see, e.g., wikipedia.org/wiki/Sequence_alignment_software, providing links to dozens of known and publicly available alignment databases and programs). Generally, for purposes herein sequence identity is determined using computer algorithms based on global alignment, such as the Needleman-Wunsch Global Sequence Alignment tool available from NCBI/BLAST (blast.ncbi.nlm.nih.gov/Blast.cgi?CMD=Web&Page_TYPE=BlastHome); LAlign (William Pearson implementing the Huang and Miller algorithm (Adv. Appl. Math. (1991) 12:337-357)); and the program from Xiaoqui Huang, available at deepc2.psi.iastate.edu/aat/align/align.html. Typically, the full-length sequence of each of the compared polypeptides or nucleotides is aligned across the full-length of each sequence in a global alignment. Local alignment also can be used when the sequences being compared are substantially the same length.

As used herein, the term “identity” represents a comparison or alignment between a test and a reference polypeptide or polynucleotide. In one non-limiting example, “at least 90% identical to” refers to percent identities from 90% to 100%, relative to the reference polypeptide or polynucleotide. Identity at a level of 90% or more is indicative of the fact that, assuming for exemplification purposes, when a test and reference polypeptide or polynucleotide with a length of 100 amino acids or nucleotides are compared, no more than 10% (i.e., 10 out of 100) of amino acids or nucleotides in the test polypeptide or polynucleotide differ from those of the reference polypeptide or polynucleotide. Similar comparisons can be made between a test and reference polynucleotide. Such differences can be represented as point mutations randomly distributed over the entire length of an amino acid sequence, or they can be clustered in one or more locations of varying length, up to the maximum allowable, e.g., 10/100 amino acid difference (approximately 90% identity). Differences also can be due to deletions or truncations of amino acid residues. Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein, a “disulfide bond” (also called an S—S bond or a disulfide bridge) is a single covalent bond derived from the coupling of thiol groups. Disulfide bonds in proteins are formed between the thiol groups of cysteine residues, and stabilize interactions between polypeptide domains, such as antibody domains.

As used herein, “coupled” or “conjugated” means attached via a covalent or noncovalent interaction.

As used herein, the phrase “conjugated to an antibody” or “linked to an antibody” or grammatical variations thereof, when referring to the attachment of a moiety to an antibody or antigen-binding fragment thereof, such as a diagnostic or therapeutic moiety, means that the moiety is attached to the antibody or antigen-binding fragment thereof by any known means for linking peptides, such as, for example, by production of fusion proteins by recombinant means, or post-translationally by chemical means. Conjugation can employ any of a variety of linking agents to effect conjugation, including, but not limited to, peptide or compound linkers, or chemical cross-linking agents.

As used herein, “antibody-dependent cell-mediated cytotoxicity,” “antibody-dependent cellular cytotoxicity” and “ADCC” refer, interchangeably, to cell-mediated reactions in which nonspecific cytotoxic cells that express Fc receptors (FcRs) (e.g., natural killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch et al. (1991) Annu. Rev. Immunol, 9:457-492. To assess ADCC activity of a molecule of interest, an in vitro ADCC assay may be performed (see, e.g., U.S. Pat. Nos. 5,500,362 and 5,821,337).Exemplary effector cells for such assays include peripheral blood mononuclear cells (PBMCs) and natural killer (NK) cells. Alternatively, or additionally, ADCC activity of the molecule of interest may be assessed in vivo, e.g., in an animal model, such as that disclosed in Clynes et al. (1998) Proc. Natl. Acad. Sci. USA 95:652-656.

As used herein, complement-dependent cytotoxicity (CDC) is an effector function of IgG and IgM antibodies. When such antibodies are bound to a surface antigen on target cell, such as a bacterial cell or viral-infected cell, the classical complement pathway is triggered by bonding protein C1q to these antibodies, resulting in formation of a membrane attack complex (MAC) and subsequent cell lysis.

As used herein, antibody-dependent cellular phagocytosis (ADCP) is a cellular process by which effector cells with phagocytic potential, such as monocytes and macrophages, internalize target cells. Once phagocytosed, the target cell resides in a phagosome, which fuses with a lysosome for degradation of the target cell via an oxygen-dependent or independent mechanism.

As used herein “therapeutic activity” refers to the in vivo activity of a therapeutic polypeptide. Generally, the therapeutic activity is the activity that is associated with treatment of a disease or condition. Therapeutic activity of a modified polypeptide can be any level of percentage of the therapeutic activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, of the therapeutic activity compared to the unmodified polypeptide.

As used herein, the term “assessing” is intended to include quantitative and qualitative determination in the sense of obtaining an absolute value for the activity of a protein, such as an antibody, or an antigen-binding fragment thereof, present in the sample, and also, of obtaining an index, ratio, percentage, visual, or other value indicative of the level of the activity. Assessment can be direct or indirect.

As used herein, a “disease or disorder” refers to a pathological condition in an organism, resulting from a cause or condition including, but not limited to, infections, acquired conditions, and genetic conditions, and characterized by identifiable symptoms.

As used herein, “treating” a subject with a disease or condition means that the subject's symptoms are partially or totally alleviated, or remain static following treatment. Hence, treatment encompasses prophylaxis, therapy and/or cure. Prophylaxis refers to prevention of a potential disease and/or a prevention of worsening of symptoms or progression of a disease. Treatment also encompasses any pharmaceutical use of any antibody or antigen-binding fragment thereof, or compositions, provided herein.

As used herein, treatment means amelioration of a symptom or manifestation of a disease, disorder, or condition.

As used herein, “prevention” or “prophylaxis,” refers to methods in which the risk of developing a disease or condition is reduced. To prevent a disease means to reduce the risk of developing the disease.

As used herein, a “pharmaceutically effective agent” includes any therapeutic agent or bioactive agent, including, but not limited to, for example, anesthetics, vasoconstrictors, dispersing agents, and conventional therapeutic drugs, including small molecule drugs and therapeutic proteins.

As used herein, a “therapeutic effect” means an effect resulting from treatment of a subject that alters, typically improves or ameliorates, the symptoms of a disease or condition, or that cures a disease or condition.

As used herein, a “therapeutically effective amount” or a “therapeutically effective dose” refers to the quantity of an agent, compound, material, or composition containing a compound that is at least sufficient to produce a therapeutic effect following administration to a subject. Hence, it is the quantity necessary for preventing, curing, ameliorating, arresting or partially arresting a symptom of a disease or disorder.

As used herein, “therapeutic efficacy” refers to the ability of an agent, compound, material, or composition containing a compound to produce a therapeutic effect in a subject to whom the agent, compound, material, or composition containing a compound has been administered.

As used herein, a “prophylactically effective amount” or a “prophylactically effective dose” refers to the quantity of an agent, compound, material, or composition containing a compound, that, when administered to a subject, will have the intended prophylactic effect, e.g., preventing or delaying the onset, or reoccurrence, of disease or symptoms, reducing the likelihood of the onset, or reoccurrence, of disease or symptoms, or reducing the incidence of viral infection. The full prophylactic effect does not necessarily occur by administration of one dose, and can occur only after administration of a series of doses. Thus, a prophylactically effective amount can be administered in one or more administrations.

As used herein, amelioration of the symptoms of a particular disease or disorder by a treatment, such as by administration of a pharmaceutical composition or other therapeutic, refers to any lessening, whether permanent or temporary, lasting or transient, of the symptoms, that can be attributed to or associated with administration of the composition or therapeutic.

As used herein, a “prodrug” is a precursor or derivative form of a pharmaceutically active substance that is less cytotoxic to tumor cells compared to the parent drug and is capable of being enzymatically activated or converted into the more active parent form (see, e.g., Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.), pp. 247-267, Humana Press, 1985).

As used herein, an “anti-cancer agent” refers to any agent that is destructive or toxic to malignant cells and tissues. For example, anti-cancer agents include agents that kill cancer cells or otherwise inhibit or impair the growth of tumors or cancer cells. Exemplary anti-cancer agents are chemotherapeutic agents.

As used herein, an “anti-angiogenic agent” or “angiogenesis inhibitor” is a compound that blocks, or interferes with, the development of blood vessels.

As used herein, a TNF-related or TNF-mediated disease refers to a disease, condition, or disorder in which TNFR1 or TNFR1 signaling plays a role in the etiology; included are diseases, disorders, and conditions in which inhibition of TNFR1 signaling can be ameliorative of a symptom of the disease, condition, or disorder.

As used herein, a “TNFR2 agonist,” or an “anti-TNFR2 agonist,” refers to compounds, including small molecules and TNFR2 antibodies or antigen-binding fragments thereof, and other polypeptides that initiate, promote, or increase activation of TNFR2 and/or potentiate one or more signal transduction pathways mediated by TNFR2. For example, TNFR2 agonists can promote or increase the proliferation of a population of Treg cells. TNFR2 agonists can promote or increase TNFR2 activation by binding to TNFR2, e.g., to induce a conformational change that renders the receptor biologically active. For example, TNFR2 agonists can nucleate the trimerization of TNFR2 in a manner similar to or that mimics the interaction between TNFR2 and its cognate ligand, TNF (TNF-α), thus inducing TNFR2-mediated signaling. TNFR2 agonists also can induce the proliferation of CD4⁺, CD25⁺, FOXP3⁺ Treg cells. TNFR2 agonists can also suppress the proliferation of cytotoxic T lymphocytes (e.g., CD8⁺ T-cells), e.g., through activation of immunomodulatory Treg cells or by directly binding to TNFR2 on the surface of an autoreactive cytotoxic T-cell and inducing apoptosis. A TNFR2 agonist antibody or fragment thereof, for use in the methods herein, can specifically bind to TNFR2, and generally is sufficiently specific so that it does not specifically binding to another receptor of the tumor necrosis factor receptor (TNFR) superfamily member, such as TNFR1.

As used herein, a TNFR2-selective agonist is a TNFR2 agonist that does not or substantially does not result in TNFR1 signaling activity.

As used herein, a Treg expander is a molecule, including small molecules and polypeptides, that increases regulatory T cells (Treg cells or Tregs), which are an immunosuppressive subpopulation of T cells with immunosuppressive properties via production of cytokines.

As used herein, an “extracellular domain” or “ECD” is the portion of a cell surface receptor that occurs on the surface of the receptor and includes the ligand-binding site(s). For purposes herein, reference to an “ECD polypeptide” includes any ECD-containing molecule, or portion thereof, as long as the ECD polypeptide does not contain any contiguous sequence associated with another domain (e.g., transmembrane domain, protein kinase domain, or others) of a cognate receptor.

As used herein, “knobs into holes” or “knobs-in-holes” (KIH), refers to multimerization domains, such as immunoglobulin Fc domains, engineered so that steric interactions between and/or among such domains, promote stable interaction, and promote the formation of heterodimers (or heteromultimers) compared to homodimers (or homomultimers) from a mixture of monomers. This can be achieved, for example, by constructing knobs or protuberances and holes or cavities in the complementary multimerizing domains. “Knobs” can be constructed by replacing small amino acid side chains from the interface of the first multimerizing domain polypeptide (e.g., first Fc monomer) with larger side chains (e.g., tyrosine or tryptophan). Compensatory “holes” of identical or similar size to the knobs optionally are created on the interface of the second complementary multimerizing polypeptide (e.g., second Fc monomer) by replacing large amino acid side chains with smaller ones (e.g., alanine or threonine).

As used herein, “tethering” refers to the interaction between two domains of a receptor monomer, whereby the monomer occurs in a conformation that renders it less available for interaction. For example, subdomain II in HER1, HER3 and HER4, can interact with subdomain IV, forming a tethered, inactive structure. When in a tethered state, a receptor or isoform thereof is less available, or is unavailable, for dimerization and/or ligand binding. The ECDs of the monomeric forms of HER1, HER3 and HER4 occur in a tethered form that exhibits lower ligand affinity than the untethered form. HER2, which lacks certain residues in subdomain IV, occurs in an untethered form and is available for dimerization with HER1, HER3 and HER4. Upon ligand binding to a tethered (monomeric) form, the tethering interaction is released, and the ECD (or receptor) is in a conformation available for dimerization, which involves interactions between domains II of two ECDs.

As used herein, the term “subject” refers to an animal, including a mammal, such as a human being.

As used herein, a “patient” refers to a human subject.

As used herein, “animal” includes any animal, such as, but not limited to, primates including humans, gorillas and monkeys; rodents, such as mice and rats; fowl, such as chickens; ruminants, such as goats, cows, deer, and sheep; pigs; and other animals. Non-human animals exclude humans as the contemplated animal. The polypeptides provided herein are from any source, animal, plant, prokaryotic and fungal. Most polypeptides are of animal origin, including mammalian origin, and generally, for therapeutic use, are human or humanized.

As used herein, a “composition” refers to any mixture. It can be a solution, suspension, liquid, powder, paste, aqueous, non-aqueous, or any combination thereof.

As used herein, a “stabilizing agent” refers to compound added to the formulation to protect either the antibody or conjugate, such as under the conditions (e.g., temperature) at which the formulations herein are stored or used. Thus, included are agents that prevent proteins from degradation from other components in the compositions. Exemplary of such agents are amino acids, amino acid derivatives, amines, sugars, polyols, salts and buffers, surfactants, inhibitors, or substrates and other agents as described herein.

As used herein, a “combination” refers to any association between or among two or more items. The combination can be two or more separate items, such as two compositions or two collections, a mixture thereof, such as a single mixture of the two or more items, or any variation thereof. The elements of a combination are generally functionally associated or related, such as elements used in a method.

As used herein, “combination therapy” refers to the administration of two or more different therapeutics, such as an anti-TNFR construct or such as an antibody or antigen-binding fragment thereof, provided herein, and one or more therapeutics or other treatment(s), such as radiation and surgery. Multiple therapeutic agents can be provided and administered separately, sequentially, intermittently, simultaneously, or in a single composition.

As used herein, a “kit” is a packaged combination that optionally includes other elements, such as additional reagents and instructions for use of the combination or elements thereof, for a purpose including, but not limited to, activation, administration, diagnosis, and assessment of a biological activity or property.

As used herein, a “unit dose form” refers to physically discrete units suitable for human and animal subjects, and packaged individually, as is known in the art.

As used herein, a “single dosage formulation” refers to a formulation for direct administration.

As used herein, a “multi-dose formulation” refers to a formulation that contains multiple doses of a therapeutic agent and that can be directly administered to provide several single doses of the therapeutic agent. The doses can be administered over the course of minutes, hours, weeks, days or months. Multi-dose formulations can allow dose adjustment, dose-pooling, and/or dose-splitting. Because multi-dose formulations are used over time, they generally contain one or more preservatives to prevent microbial growth.

As used herein, an “article of manufacture” is a product that is made and sold.

As used throughout this application, the term is intended to encompass any of the compositions provided herein contained in articles of or for packaging.

As used herein, a “fluid” refers to any composition that can flow. Fluids thus encompass compositions that are in the form of semi-solids, pastes, solutions, aqueous mixtures, gels, lotions, creams and other such compositions.

As used herein, an isolated or purified polypeptide or protein (e.g., an isolated antibody or antigen-binding fragment thereof), or biologically-active portion thereof (e.g., an isolated antigen-binding fragment), is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis, and high performance liquid chromatography (HPLC), used by those of skill in the art to assess such purity, or sufficiently pure such that further purification does not detectably alter the physical and chemical properties, such as enzymatic and biological activities, of the substance.

Methods for purification of the compounds to produce substantially chemically pure compounds are known to those of skill in the art. A substantially chemically pure compound, however, can be a mixture of stereoisomers. In such instances, further purification might increase the specific activity of the compound.

As used herein, a “cellular extract” or “lysate” refers to a preparation or fraction which is made from a lysed or disrupted cell.

As used herein, a “control” refers to a sample that is substantially identical to the test sample, except that it is not treated with a test parameter, or, if it is a plasma sample, it can be from a normal volunteer not affected with the condition of interest.

A control also can be an internal control.

As used herein, the singular forms “a,” “an” and “the” include plural referents unless the context clearly dictates otherwise. Thus, for example, reference to a polypeptide, containing “an immunoglobulin domain” includes polypeptides with one or a plurality of immunoglobulin domains.

As used herein, the term “or” is used to mean “and/or” unless explicitly indicated to refer to alternatives only, or the alternatives are mutually exclusive.

As used herein, ranges and amounts can be expressed as “about” a particular value or range. “About” also includes the exact amount. Hence “about 5 amino acids” means “about 5 amino acids” and also “5 amino acids.” For particular parameters about is a range within experimental error or a range acceptable to one of skill in the art for a particular parameter.

As used herein, “optional” or “optionally” means that the subsequently described event or circumstance does or does not occur, and that the description includes instances where said event or circumstance occurs and instances where it does not. For example, an optionally variant portion means that the portion is variant or non-variant.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

For clarity of disclosure, and not by way of limitation, the detailed description is divided into the subsections that follow.

### B. Overview of Constructs and Methods

Autoimmune disease occurs when the body's immune system attacks itself. The resulting inflammation and tissue destruction is initiated by an inflammatory hormone called tumor necrosis factor (TNF). There are more than 100 types of autoimmune disease; overall, about 75% of those with an autoimmune disease are women. Prior drugs for autoimmune disease have adverse side effects, including infections, heart problems, and other diseases and disorders,

TNF interacts with immune cells via two receptors, TNFR1, which is overactive in autoimmune disease, and TNFR2 which suppresses autoimmune disease, but is muted when TNFR1 is overactive. TNF blockers, such as infliximab (sold as Remicade®), adalimumab (sold as Humira®), and etanercept (sold as Enbrel®) block TNFR1 and TNFR2, resulting in the adverse side effects. Constructs provided herein address this problem. Constructs provided herein shut down only TNFR1, which leads to increased TNFR2 activity, thereby not only treating autoimmune disease symptoms, but providing improved treatment and reduced or no adverse side effects because TNFR2 activity is not blocked. Provided herein a variety of constructs that address the problems with the prior art TNF blockers. Types of constructs identified by their activity, and detailed and provided herein, are summarized in the following table:

| Type of Construct | Disease to be Treated | Action |
|---|---|---|
| TNFR1 | Autoimmune disease | Specific blockade of TNFR1; |
| Antagonists | and acute | spares TNFR2 |
| | inflammation | |
| TNFR2 | Cancer checkpoint | Inhibition of tumoral |
| Antagonists | inhibitor | suppressor Treg function |
| | | thus increases active |
| | | immunity |
| TNFR2 Agonists | Inflammation and | Induces proliferation of |
| | fibrosis | Treg to reduce inflammation |

Provided are constructs for treatment of TNF-mediated diseases, disorders, and conditions, or diseases, disorders, and conditions in which TNF plays a role in the etiology, or in which interference with TNFR1 signaling has an ameliorative effect. For example, the TNFR1 antagonists can be used for treatment of a variety of disorders, including autoimmune disorders, and also diseases and conditions, such as endometriosis, brain fog, such as from chemotherapy and COVID, Alzheimer's disease, acute inflammation, such as results from infection by influenza viruses, and SARS-COV2, which results in long-lasting or permanent damage to the lungs, kidneys, and other tissues. Because of the adverse effects and consequent safety concerns with prior TNF blockers, they cannot be used for most of these indications. The TNFR1 antagonist constructs provided herein can be used. These constructs as described herein are monovalent in that they only inhibit TNFR1 and do not cause receptor clustering, they are specific, non-immunogenic, and have a half-life of at least about 3-4 weeks, permitting approximately once-a-month dosing.

Hence, provided are TNFR1 antagonist constructs, TNFR2 agonist constructs, and multi-specific, such as bi-specific constructs that include TNFR1 antagonist and TNFR2 agonist activity. The constructs include at least one moiety that specifically interacts with TNFR1 or TNFR2, and, generally, a further moiety that modulates the interaction directly or indirectly or that provides a pharmacological (pharmacodynamic or pharmacokinetic or both) property to the construct. Hence a construct as provided herein includes at least two moieties: a binding moiety that interacts with TNFR1 or TNFR2, and a second moiety that modulates or alters pharmacological properties or activities of the construct or the binding moiety.

Among the constructs provided herein are those that are antagonists of TNFR1 activity. The TNFR1 antagonist constructs contain a portion that binds to or interacts with TNFR1 and inhibits TNFR1-mediated signaling, and a second portion that confers additional properties, such as extended serum half-life, elimination of ADCC and/or CDC activity, and modulation of interaction with particular receptors. The TNFR1 antagonists and constructs also include modification(s) so that they have none or reduced immunogenicity, particularly in a human, and also can include modifications to eliminate or reduce binding to pre-existing antibodies.

The TNFR1 antagonist constructs, are selected to specifically bind to TNFR1, and to have minimal or no binding to TNFR2 or no TNFR2 antagonist activity. Thus, the constructs only modulate TNFR1. In some embodiments, the TNFR1 antagonist constructs are selected to also have or to be linked to a second domain or moiety that has TNFR2 agonist activity. The TNFR1 constructs, include those that are designed or selected to interact with TNFR1 with affinity, such as K_{d}<50 nM or <10 nM or <5 nM, and particularly with higher affinity (as K_{d}<1 nM or <0.1 nM or higher affinity) and/or potent inhibition of TNFR1 signaling (e.g., IC₅₀50 nM or <10 nM or <5 nM or <3 nM or, 1 nM or <0.5 nM).

Also provided are multi-specific, such as bi-specific, constructs that contain a TNFR1 antagonist moiety, linked directly, or via a linker, to a TNFR2 agonist moiety. The linker provides advantageous properties to the molecules, such as, for example, increased serum half-life, increased stability, proper three-dimensional structure and flexibility, and improved pharmacological properties. These constructs solve problems associated with the administration of other therapies, such as anti-TNF therapies (“TNF Blockers,” (examples include etanercept (Enbrel®), adalimumab (Humira®), Infliximab)), because these constructs increase the specificity of TNFR1 inflammatory blockade and result in conservation or amplification of TNFR2 function, which is a natural immunosuppressor, at least in part by up-regulation of immunosuppressive Tregs, and the induction of protective and anti-inflammatory signaling pathways. In addition, TNF Blockade resulting in inhibition of TNFR2 function also reduces the T cell-induced monocyte activation leading to increased possibility of opportunistic infections (see e.g., Rossel et al. (2007) J. Immunol. 179:4239-48).

There are numerous differences between the activity of exemplary TNFR1 antagonist constructs provided herein and existing approved TNF Blockers: TNF Blockers, such as etanercept, adalimumab, infliximab, is that they are not specific for TNFR1. Other blockers, such as like 1L6, IL17, IL23 only block their own part of the cytokine cascade, not the whole thing. Existing TNF blockers have the same mechanism of action for TNFR1 and TNFR2, thereby blocking the activity of both. JAK inhibitors pose similar problems; they have inflammatory and anti-inflammatory activities. For example, the inflammatory cytokine Ill is not blocked by JAK inhibitors, the inflammatory cytokine IL6 is blocked by JAK inhibitors (a second line use for rheumatoid arthritis treatment), and IL10, which is anti-inflammatory, is not blocked by JAK inhibitors. Constructs provided herein, in contrast, combine the effectiveness of TNFR1 and TNF inhibitor therapies with the benefits of TNFR2 agonists that eliminate or reduce the adverse effects of anti-TNFR1/anti-TNF therapies, and also contribute additional therapeutic modalities advantages, including the up-regulation of immunosuppressive Tregs, and the induction of protective and anti-inflammatory signaling pathways.

The TNFR1 antagonist constructs contain one or more TNFR1 inhibitors, one or more linkers, and one or more activity modifiers. For example, the structure of the TNFR1 antagonist constructs provided herein can be represented by the formulae 1:

(TNFR1 inhibitor)ₙ-linkerₚ-(activity modifier)_{q}, Formula 1a, or

(activity modifier)g-linkerₚ-(TNFR1 inhibitor)ₙ Formula 1b, where:

each of n and q is an integer, and each is independently 1, 2, or 3; p is 0, 1, 2 or 3; and an activity modifier is a moiety, such as a polypeptide, such as albumin, or an Fc that is modified to have reduced or no ADCC activity, that increases serum half-life of the TNFR1 inhibitor; and the TNFR1 inhibitor is a molecule, such as a polypeptide or small drug molecule that binds to TNFR1 and inhibits its activity, such as signaling activity. The activity modifier is not a human serum albumin antibody or an unmodified single Fc. Activity modifiers include modified Fc regions, such as Fc modified to eliminate ADCC and/or CDC activity, Fc dimers, and other antibody domains. The linkers include chemical linkers, and polypeptides, such as GS linkers, and hinge regions, such as from antibodies, so that the constructs include chemical conjugates, fusion proteins, and combinations of both.

Also provided are multi-specific constructs. The structure of the multi-specific, such as, bi-specific, constructs provided herein is represented by the following formula (Formula 2):

(TNFR1 inhibitor)ₙ-(activity modifier)ᵣ₁-(Linker(L))ₚ-(activity modifier)ᵣ₂-(TNFR2 agonist)_{q},

where n=1, 2, or 3, p=1, 2, or 3, q=0, 1 or 2, and each of r1 and r2 is independently 0, 1, or 2. As with the constructs of formulae 1 the order of components can be varied and there can be additional linkers as needed. The constructs can include additional linkers as required for conferring properties such as flexibility. Each linker can contain a plurality of components. Formula 2 also can include an activity modifier in place of or in addition to a linker. Activity modifiers and linkers include, an Fc or and Fc with a hinge region, or an Fc with a GS linker, or other combinations of components. The Fc in these constructs include unmodified Fc regions; the linkers are as described above, and detailed below.

Also provided are TNFR2 agonist constructs that have formulae 3:

(TNFR2 agonist)ₙ-linkerₚ-(activity modifier)_{q}, formula 3a, or

(activity modifier)g-linkerₚ-(TNFR2 agonist)ₙ, formula 3b,

where n, p and q are as set forth for formula 1, and the linkers and activity modifier are as described in formula 1.

The components, which are discussed in detail in the following sections, of formulae 1-3 can be polypeptides or other molecules, such as small drugs that specifically bind to or interact with the targeted receptor. Each component of the constructs/molecules provided herein is described in turn in sections below.

The properties of each component of the constructs provided herein is discussed in detail in sections below. The components of the constructs, thus, include, but are not limited to, the following components, which are discussed in detail in Sections that follow:
1. TNFR1 Antagonists
2. TNFR2 Agonists
3. Linkers
   a. Glycine-Serine Linkers
   b. Hinge Regions
   c. chemical linkers
4. Activity modifiers
   a. Modified Fcs
   b. Polypeptides and other moieties that confer improved or altered pharmacological properties, such as increased serum half-life, resistance to degradation by endogenous proteases, and other such properties.
      Other constructs, detailed in Sections that follow, also are provided.

The constructs are used in methods of treatment of diseases, disorders, and conditions in which TNF in a pathologic modifier of the disease, condition, or disorder, such that inhibition TNFR1 signaling is reduced or inhibited, and/or in which inhibition of TNF or TNFR1 signaling can suppress or cause regression of the disease, disorder, or condition, and/or in which inhibition ameliorates a symptom of the disease, disorder, and/or condition. Such diseases, conditions, and disorders, which include inflammatory diseases, including autoimmune diseases, are discussed in the section that follows.

Also provided are pharmaceutical compositions for use in the methods and uses, and nucleic acids and vectors for producing constructs that include polypeptides and those that are fusion proteins. The following sections describe diseases, disorders, and conditions, TNFR1/TNFR2 activities and their roles in the diseases, disorders, and conditions, existing treatments for the diseases, disorders, and conditions, constructs and components thereof that are provided herein, methods of producing the constructs, pharmaceutical compositions containing the constructs and/or encoding nucleic acids, and methods of treatment.

### C. Tumor Necrosis Factor (TNF) and Chronic Inflammatory and Autoimmune Diseases and Disorders

This section describes the role that tumor necrosis factor (TNF) and/or its receptors play in inflammatory and autoimmune diseases, particulars of exemplary diseases, and problems with existing therapies, and shows how the constructs provided herein address these problems.

TNF-α acts as a trimer and has two high affinity receptors:TNFR1 and TNFR2. TNFR1 is the first to be activated by TNF. It is ubiquitously expressed, and is involved in autoimmune disorders, bone loss, cytokine release syndrome, inflammatory disorders, Alzheimer's Disease, acute and long-term Covid, and other diseases, disorders, and conditions. TNFR2 is slower to activate; it has higher expression in myeloid and regulatory TT cells; it is associated with immunosuppression, with tissue repair, including cardiac; and is involved in macrophage surveillance/killing of invading pathogens and incipient tumor cells. As described throughout the disclosure herein, prior art TNF blockers inhibit both receptors, thereby compromising advantageous TNFR2 activities.

Anti-TNF-α treatments of the prior art block TNFR1 (pro-inflammatory) and TNFR2 (anti-inflammatory), which results in serious side effects including a risk of infection, cardiovascular abnormalities and cancer. The constructs provided herein are TNFR1-specific antagonists that do not inhibit TNFR2 and do not have TNFR1 agonist activity. These constructs can be used for treatment of a variety of diseases, disorders, and conditions in which TNF plays a role. These include rheumatoid arthritis, other autoimmune diseases, and cognitive disorders. By sparing TNFR2 function, a specific TNFR1 antagonist provides a safer treatment with reduced side effects, and a longer duration of symptom relief, thus benefiting a broader patient population.

TNFR1 has ubiquitous expression, dominant inflammatory signaling and necrosis/apoptosis, and is associated with arthritis, cytokine storm syndromes, heart failure, Alzheimer's disease and others. TNFR2 has higher expression in myeloid and regulatory T-cells. TNFR2 is slow to activate and is associated with being anti-inflammatory, immunoregulatory, it also promotes Treg function, tissue repair and proliferation. Prior art clinical TNF blockers inhibit both TNFR1 and TNFR2, thus compromising a natural mechanism of return to homeostasis after an immune insult activates TNF production.

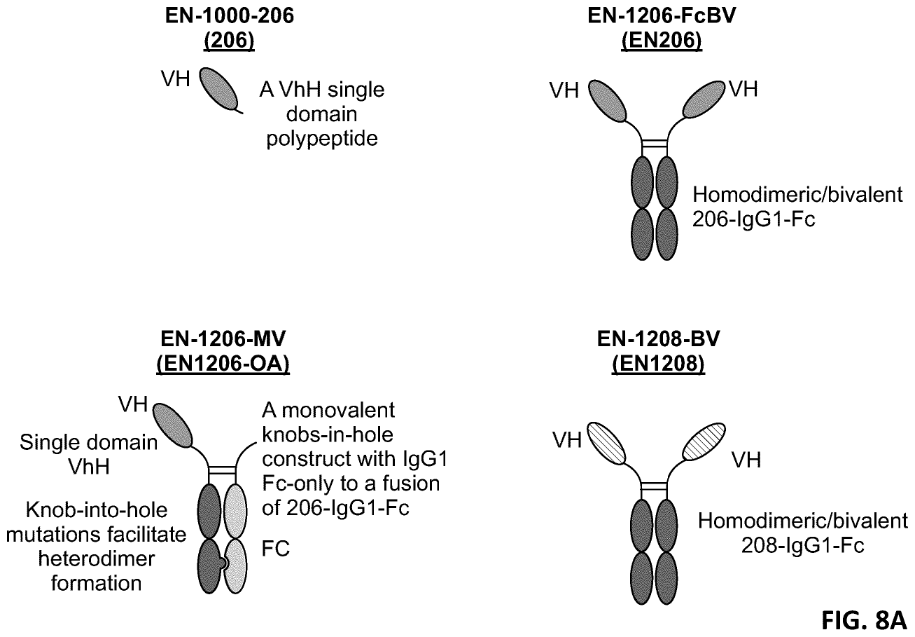

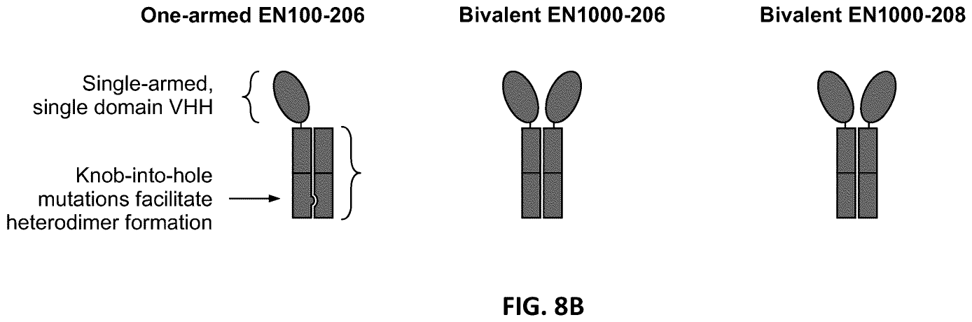

Constructs exemplified herein in the Examples, including constructs designated EN-1206 (EN-1206-MV (monovalent) and -BV (bivalent)), are shown to be more potent than etanercept and have no detectable agonist activity. Constructs provided herein, such as the EN-1206 molecules, are designed to minimize FcR binding and to enhance FcRn recycling. The constructs herein can be engineered for monthly subcutaneous dosing. Exemplary constructs are depicted in FIGS. 8A and 8B.

In contrast to the 1206 construct, the 1208 construct has agonist activity, and thus, is not included among the constructs provided herein as having the ability to antagonize TNFR1, while not inhibiting TNFR2, and, not agonizing TNFR1. The dAbs (VHH) employed in the construct provided herein are those that bind to the same epitope as the 1206 constructs and do not bind the epitope to which 1208 binds.

Components of the constructs are detailed below and in the Examples.

Constructs contemplated herein are those in which the VHH component, a dAb, such as those exemplified herein (see e.g., SEQ ID Nos: 54-672), is one that binds TNFR1, does not bind TNFR2, and is identified by assay or by identifying those that bind the same epitope as the 1206 constructs.

### 1. Tumor Necrosis Factor (TNF)

Tumor necrosis factor (TNF; see e.g., SEQ ID NO:1; also referred to as TNF alpha, TNF-α, or TNFα) is a pleiotropic, proinflammatory cytokine that is associated with inflammatory and immuno-regulatory activities, including the regulation of tumorigenesis/cancer, host defense against pathogenic infections, apoptosis, autoimmunity, and septic shock, and that plays an important role in the coordination of innate and adaptive immune responses, as well as organogenesis, particularly of the lymphoid organs. In humans, TNF is produced primarily by macrophages, and also can be produced by monocytes, dendritic cells (DCs), B cells, T cells, fibroblasts and other cell types. It is produced as a homotrimeric membrane-bound protein containing 233 amino acids (26 kDa) that can be cleaved by the protease TACE (TNF alpha converting enzyme; also known as ADA17) to release soluble TNF, which contains 157 amino acids (17 kDa); membrane-bound and soluble forms of TNF are biologically active. Transmembrane human TNF contains 233 amino acids, and contains a cytoplasmic domain, corresponding to residues 1-35, a transmembrane domain, corresponding to residues 36-56, and an extracellular domain, corresponding to residues 57-233, with reference to SEQ ID NO:1. The soluble form of TNF corresponds to amino acid residues 77-233, as set forth in SEQ ID NO:1 (see, SEQ ID NO:2 for the sequence of amino acid residues of soluble TNF).

Uncontrolled production of TNF is associated with several inflammatory and autoimmune diseases and conditions, including, for example, septic shock, rheumatoid arthritis, psoriasis, psoriatic arthritis, ankylosing spondylitis, juvenile idiopathic arthritis, and inflammatory bowel disease (IBD). The overexpression of TNF also has been associated with neurodegenerative diseases and conditions, such as, for example, Alzheimer's disease, Parkinson's disease, stroke and multiple sclerosis. Additionally, TNF promotes osteoclastogenesis, and overproduction of TNF is associated with bone loss. In rheumatoid arthritis (RA), TNF is over-expressed in synovial fluids and in the synovial membrane, while expression of TNF receptors (TNFRs) is up-regulated in the synovial membrane. For example, overexpression of human TNF in mice results in the development of spontaneous RA-like lesions in the joints with the formation of hyperplastic synovial membranes and the destruction of cartilage and bone (see, e.g., Blüml et al. (2010) Arthritis & Rheumatism 62(6):1608-1619; Keffer et al. (1991) EMBO J. 10(13):4025-4031; Esperito Santo et a. (2015) Biochem. Biophys. Res. Commun. 464:1145-1150; Blüml et al. (2012) International Immunology 24(5):275-281; Dong et al. (2016) Proc. Natl. Acad. Sci. USA 113(43):12304-12309).

As discussed further below, TNF signals through two high-affinity, specific receptors, TNFR1 and TNFR2; TNFR1 is associated with detrimental inflammatory processes, while TNFR2 is associated with beneficial immuno-regulatory processes. It has been shown that membrane-bound TNF primarily activates TNFR2, while soluble TNF primarily activates TNFR1 (Blüml et al. (2010) Arthritis & Rheumatism 62(6):1608-1619). Soluble TNF (solTNF; corresponding to residues 77-233 of SEQ ID NO:1; see, also, the sequence set forth in SEQ ID NO:2), which is involved in paracrine signaling (primarily via TNFR1), is associated with chronic inflammation, whereas transmembrane TNF (tmTNF), which acts via cell-to-cell contact to induce juxtacrine signaling (primarily via TNFR2), is associated with the resolution of inflammation and with the induction of immunity against pathogens, such as Listeria monocytogenes and Mycobacterium tuberculosis (Zalevsky et al. (2007) J. Immunol. 179:1872-1883). Thus, TNF signaling through TNFR1 and TNFR2, effects different outcomes, depending on the receptor type.

Due to the association between TNF overexpression and the development of inflammatory and autoimmune diseases and conditions, the blockade of TNF has been used in the treatment of various such diseases and conditions, including, but not limited to, rheumatoid arthritis (RA), psoriasis, psoriatic arthritis, ankylosing spondylitis, juvenile idiopathic arthritis (JIA), and inflammatory bowel disease (IBD; e.g., Crohn's disease, ulcerative colitis). The use of TNF blockers, which block TNF and prevent signaling via both TNFR1 and TNFR2, is associated with an increased risk of serious infections, such as tuberculosis and listeriosis, due to immunosuppression. TNF blockers not only block detrimental inflammatory signaling via TNFR1, but also block beneficial, immune-regulatory signaling via TNFR2. As a result, the use of TNF blockers, particularly in the case of chronic diseases/conditions that require long-term administration, such as arthritis or IBD, can be limited. Approximately one-third of RA patients are non-responsive, or therapeutic benefits are not sustained, with the use of anti-TNF therapies. Thus, there is a need for therapies with improved therapeutic efficacy and safety, particularly therapies that block the inflammatory effects of TNFR1 signaling, but maintain, or boost, the beneficial anti-inflammatory effects of TNFR2 signaling. Such therapies are provided herein.

### 2. Tumor Necrosis Factor Receptors (TNFRs)

Homotrimers of TNF bind to and signal through two specific, high-affinity homotrimeric receptors, TNFR1 (TNF receptor type 1; also known to as TNFRI, p55, p60, CD120a, TNF receptor superfamily member 1A, and TNFRSF1A), and TNFR2 (TNF receptor type 2; also known as TNFRII, p75, p80, CD120b, TNF receptor superfamily member 1B, and TNFRSF1B). TNFR1 is expressed by all nucleated cell types; TNFR2 expression is restricted to immune cells (e.g., monocytes, macrophages, activated T cells, regulatory T cells (Tregs), B cells and natural killer (NK) cells), endothelial cells, particular central nervous system (CNS) cells, and particular cardiac cells. TNFR2 expression on Tregs is induced upon T-cell receptor activation.

In vivo, TNFR1 and TNFR2 exist as membrane-bound receptors, and as soluble, “decoy” (i.e., non-signaling) receptors, following shedding from cell surfaces. Soluble TNF preferentially/selectively binds to TNFR1; binding of the membrane-bound and soluble forms of TNF, however, activates TNFR1. The primary ligand for TNFR2 is membrane-bound TNF. Soluble TNF does not fully activate TNFR2, but the soluble form of TNFR2 (following TNFR2 shedding) has a high binding affinity for TNF, allowing it to scavenge and inhibit TNF from binding membrane-bound, signaling receptors, which contributes to the anti-inflammatory effects of TNFR2. Membrane-bound TNFR2 binds TNF with rapid on and off kinetics, allowing TNFR2 to concentrate TNF on cell surfaces and pass the ligand to TNFR1, which mediates TNFR1 signaling. Each of TNFR1 and TNFR2 contains extracellular, transmembrane and cytoplasmic domains. The extracellular domains of TNFR1 and TNFR2 contain four cysteine-rich domains (CRDs) that are required for ligand binding. The intracellular domains of TNFR1 and TNFR2 initiate different signaling cascades, and mediate different effector functions, in response to TNF ligand binding.

TNFR signaling abnormalities are associated with several autoimmune diseases, and the administration of TNF can be used as a treatment strategy for such diseases. For example, low dose TNF selectively destroys autoreactive T cells in blood samples from type I diabetes and scleroderma patients, and in an animal model of Sjogren's syndrome. The administration of TNF can result in systemic toxicity, for example, in cancer patients with high TNF levels. As described herein, the toxicity results from the ubiquitous cellular expression of TNFR1; as described herein, agonizing TNFR2 is a safer therapeutic option than administration of TNF, due to its more restricted cellular expression. Promotion of TNF signaling via TNFR2 can be effected by administering a TNFR1 antagonist (see, e.g., Faustman et al. (2013) Front. Immunol. 4:478).

a. TNFR1

Human TNFR1 (see, SEQ ID NO:3), is the major inflammatory receptor, and accounts for the majority of the proinflammatory, cytotoxic and apoptotic effects attributed to TNF. Human TNFR1 is a homotrimeric receptor, and its binding by TNF induces a pro-inflammatory response (see, e.g., Morton et al. (2019) Sci Signal. 12(592):eaaw2418, for a description of TNFR1 signaling). TNFR1 contains 455 amino acid residues; residues 1-29 correspond to the signal peptide, residues 30-211 correspond to the extracellular domain, residues 212-232 correspond to the transmembrane domain, and residues 233-455 correspond to the cytoplasmic domain. Within the extracellular domain, TNFR1 contains cysteine-rich domains (CRDs) 1-4, corresponding to amino acid residues 43-82, 83-125, 126-166 and 167-196 of SEQ ID NO:3, respectively. CRDs 2 and 3 contact bound TNF, and CRD1, particularly amino acid residues 30-82 with reference to SEQ ID NO:3, forms the pre-ligand binding assembly domain (PLAD), a hemophilic interaction motif that is necessary for ligand binding and receptor function. The cytoplasmic domain contains a death domain (corresponding to residues 356-441 of SEQ ID NO:3) that binds to the TNFR1-associated death domain (TRADD) and the Fas-associated death domain (FADD) following the binding of TNF to TNFR1, resulting in signaling pathways that activate caspases and induce apoptosis. The binding of TNF to TNFR1 also initiates proinflammatory cascades through MAPK (mitogen-activated protein kinase; e.g., p38, JNK, ERK) and NF-κB (nuclear factor kappa-light-chain-enhancer of activated B cells) signaling pathways. TNFR1 plays a role in lymphatic organogenesis and in the immune response to pathogens, and is the primary receptor associated with host antiviral defense mechanisms. It has been shown that mycobacterial containment depends on TNF-derived signals, and that patients treated with TNF-blockers can suffer from endogenous reactivation of latent tuberculosis.

TNFR1, which primarily is involved in pro-inflammatory signaling, is the driving force in the development of arthritis. For example, knockout of TNFR1 in mice, as well as silencing of TNFR1 expression by RNA interference, results in the attenuation of collagen-induced arthritis (CIA), an animal model of arthritis. TNFR1 deficient mice that overexpress TNF are protected from the development of arthritis, and the reintroduction of TNFR1 on mesenchymal cells results in the development of TNF-dependent arthritis. Additionally, TNFR1 enhances the generation of osteoclasts, resulting in local bone destruction, and it has been shown that the lack of TNFR1 on hematopoietic cells attenuates bone destruction in a model of erosive arthritis. TNFR1 also has been associated with cardiotoxic effects in TNF-induced models of heart failure and myocardial infarction, and has been shown to promote neurodegeneration in an animal model of retinal ischemia (see, e.g., Schmidt et al. (2013) Arthritis & Rheumatism 65(9):2262-2273; Goodall et al. (2015) PLoS ONE 10(9):e0137065; McCann et al. (2014) Arthritis & Rheumatology 66(10):2728-2738; Ruspi et al. (2014) Cellular Signaling 26:683-690; Faustman and Davis (2013) Front. Immunol. 4:478; Blüml et al. (2012) International Immunology 24(5):275-281; Dong et al. (2016) Proc. Natl. Acad. Sci. USA 113(43):12304-12309).

b. TNFR2

Human TNFR2 (see, SEQ ID NO:4) contains 461 amino acid residues; residues 1-22 correspond to the signal peptide, residues 23-257 correspond to the extracellular domain, residues 258-287 correspond to the transmembrane domain, and residues 288-461 correspond to the cytoplasmic domain. TNFR2, which, unlike TNFR1, lacks a death domain, has a TNF receptor-associated factor 2 (TRAF2) binding site. TNFR2 signaling via TRAF2 promotes cell survival and proliferation through NF-κB and activator protein 1 (AP1) activation, and has been associated with PI3K-PKB/Akt-mediated repair and migration. As discussed elsewhere herein, TNF signaling via TNFR2 also promotes the expansion and activation of regulatory T cells (Tregs), which play an important role in the suppression of inflammatory and autoimmune diseases and disorders. TNFR2 signaling has been implicated in repair and regeneration in models of wound healing and myocardial infarction, while knockout of TNFR2 in a mouse model of erosive arthritis results in joint inflammation and bone destruction.

TNFR2, which primarily is involved in anti-inflammatory signaling, has been associated with neuro-, cardio-, gut- and osteo-protective effects. TNFR2 exhibits anti-inflammatory and protective effects; these effects have been demonstrated, for example, in experimental autoimmune encephalomyelitis (EAE), experimental colitis, heart failure/heart disease, myocardial infarction, inflammatory arthritis, demyelinating and neurodegenerative disorders, and infectious disease. For example, activation of TNFR2 by TNF inhibits seizures, attenuates cognitive dysfunction following brain injury, promotes survival following myocardial infarction in mice, protects against myocardial ischemia/reperfusion injury, and reduces remodeling and hypertrophy following heart failure. TNFR2 agonism also is associated with pancreatic regeneration, remyelination, survival of neuron subtypes, and stem cell proliferation. TNFR2 agonism selectively destroys autoreactive T cells, but not healthy cells, in blood samples from patients with type I diabetes, multiple sclerosis, Graves' disease and Sjogren's syndrome. In animal models of type I diabetes, elimination of autoreactive T cells using low-dose TNF results in the regeneration of pancreatic tissue. TNF signaling through TNFR2 has been shown to induce regeneration of oligodendrocyte precursors in myelin, and thus, can be of use for the treatment of demyelinating disorders, such as multiple sclerosis (MS). TNFR2 also has been shown to promote neuroprotection in an animal model of retinal ischemia.

TNFR2 also regulates osteoclastogenesis. Osteoclasts are a type of bone cells that break down bone tissue; the regulation of osteoclastogenesis is important for maintaining bone mass, and protecting against joint inflammation and erosive destruction. Mice lacking TNFR2 display enhanced osteoclastogenesis, worsening TNF-driven arthritis, and local bone destruction. The lack of TNFR2 in an animal model of erosive arthritis results in disease progression, and TNFR2-deficient mice overexpressing TNF develop aggravated arthritis and joint destruction compared with control mice. Expression of TNFR2 on hematopoietic cells attenuates TNF-driven arthritis, while the loss of TNFR2 on hematopoietic cells increases the recruitment of inflammatory cells to the synovial membrane. In experimental colitis, the lack of TNFR2 expression on CD4⁺ T cells accelerates the onset of disease and increases the severity of inflammation, while in experimental autoimmune encephalitis (EAE), symptoms are exacerbated in TNFR2-deficient mice (see, e.g., Schmidt et al. (2013) Arthritis & Rheumatism 65(9):2262-2273; Goodall et al. (2015) PLoS ONE 10(9):e0137065; McCann et al. (2014) Arthritis & Rheumatology 66(10):2728-2738; Ruspi et al. (2014) Cellular Signaling 26:683-690; Faustman and Davis (2013) Front. Immunol. 4:478; Blüml et al. (2012) International Immunology 24(5):275-281; Dong et al. (2016) Proc. Natl. Acad. Sci. USA 113(43):12304-12309). Polymorphisms in the TNFR2 gene are correlated with a variety of autoimmune diseases, including, for example, RA, Crohn's disease, systemic lupus erythematosus, ankylosing spondylitis, inflammatory bowel disease, ulcerative colitis and scleroderma; the polymorphisms hinder the binding of TNF to TNFR2, which limits activation of NF-κB and hampers TNFR2 signaling pathways in Tregs (see, e.g., Yang et al. (2018) Front. Immunol. 9:784).

TNFR1 contains an intracellular death domain and can activate apoptotic and/or inflammatory pathways, while TNFR2 binds TRAFs and can activate the canonical and non-canonical NF-κB pathways to control cell survival and proliferation. In general, cells that express TNFR2 also express TNFR1, at varying ratios, depending on the cell type and function. Since TNFR1 signaling generally induces cell death, whereas TNFR2 signaling generally induces cell survival, the ratio of their co-expression on cells shifts the balance towards apoptosis or cell survival. As discussed above and elsewhere herein, it has been shown that TNFR1 is the primary TNF receptor involved in the pathogenesis of RA, while TNFR2 plays an immunoregulatory role. Both receptors, however, are involved in mediating the antiviral activity of TNF. Animal disease models, for example, show that TNFR1 is associated with inflammatory neurodegeneration, while TNFR2 is associated with neuroprotection.

The selective inhibition of TNFR1, or the selective activation of TNFR2, has been demonstrated in a mouse model of NMDA-induced acute neurodegeneration, by administration of either ATROSAB (Antagonistic TNF Receptor One-Specific Antibody), a TNFR1-selective antagonistic IgG1 antibody, or EHD2-scTNF_{R2}, an agonistic TNFR2-selective TNF mutein (i.e., mutated protein). EHD2-scTNF_{R2} contains a covalently stabilized human TNFR2-selective single-chain TNF trimer with the mutations D143N/A145R (residue numbering with respect to soluble TNF as set forth in SEQ ID NO:2, and corresponding to D219N and A221R, respectively, with respect to SEQ ID NO:1; these mutations abrogate affinity for TNFR1), fused to the dimerization domain EHD2, which is derived from the heavy chain C_{H}2 domain of IgE and creates a disulfide bonded dimer that contains hexameric TNF domains. Simultaneous injection of NMDA and ATROSAB, or NMDA and EHD2-scTNFR₂, into the nucleus basalis magnocellularis results in significant but incomplete neuroprotective effects compared with controls, in an in vivo mouse model. The incomplete nature of these responses was due to the agonistic activity of ATROSAB, a byproduct of the bivalent antibody inducing aberrant receptor clustering and activation (Richter et al. (2013) PLoS One 8(8):e72156). Similarly, the EHD2-scTNFR2 is immunogenic in humans because of its multiple fusion partners, and an immune response to the IgE fragments result in an autoimmune reaction in toxicology studies (see, e.g., Weeratna et al. (2016) Immun. Inflamm. Dis. 4(2):135-147). Therefore, improved TNFR1 antagonists and improved TNFR2 agonists are needed that overcome these limitations.

### 3. Regulatory T Cells (Tregs) and Their Role in the Autoimmune Microenvironment

Regulatory T cells (Treg cells or Tregs) are an immunosuppressive subpopulation of T cells with immunosuppressive properties via production of cytokines. These include transforming growth factor beta, interleukin 35, and interleukin 10. Induction of Treg function can inhibit several pathologies. Induction can enhance success of transplantation, suppress allergy, control responses, such as severe acute respiratory syndrome, to infectious disease and autoimmunity. Tregs suppress and/or downregulate the induction and proliferation of effector T cells (Teffs), modulate the immune system, maintain immune homeostasis and tolerance to self-antigens, and can prevent the development of autoimmune disease and tissue destruction. Tregs express, among other markers, CD4, CTLA-4, CD25 (also known as IL-2 receptor alpha chain or IL2RA), and FOXP3 (transcription factor forkhead box P3), and express TNFR2 at a tenfold higher density than they express TNFR1. TNFR2 is expressed by only a subpopulation of Tregs, which is the maximally suppressive subset; this subset contains TNFR2-expressing CD4⁺FoxP3⁺ Tregs. TNF, via TNFR2 signaling, promotes Treg cell proliferation, up-regulation of FoxP3 expression, and Treg cell suppressive activity/function. The autoimmune microenvironment contains more autoreactive CD8⁺effector T cells than immunosuppressive CD4⁺Tregs, resulting in tissue destruction. As a result, preservation of TNFR2 function, or enhanced TNFR2 function, which expands Tregs and eliminates autoreactive T cells, restores the immune balance (see, Sharma et al. (2018) Front Immunol. 9:883). For these reasons, and others described below, pharmacological retention of Treg function by selective inhibition of TNFR1, possibly together with TNFR2 stimulation (agonism), would improve outcomes in many acute and chronic inflammatory conditions (severe acute respiratory syndrome, autoimmune diseases).

In addition to up-regulating the expression of TNFR2 on Tregs, TNF also up-regulates the Treg surface expression of other co-stimulatory members of the TNF receptor superfamily (TNFRSF), such as 4-1BB and OX40, result in the optimal activation and proliferation of Tregs, and in the attenuation of excessive inflammatory responses. Neutralization of TNF (blocking TNFR2) blocks in vivo expansion of Tregs (e.g., Hamano et al. (2011) Eur. J. Immunol. 41:2010-2020).

In comparison to CD4⁺FoxP3⁺ conventional T cells, CD4⁺FoxP3⁺Tregs constitutively express TNFR2, promoting Treg cell activation, expansion and survival. TNF signaling through TNFR2 (i.e., TNFR2 agonism) promotes the activation and expansion of Tregs, while TNFR2 antagonism results in Treg contraction. For example, TNFR2 agonism selectively kills autoreactive T cells and expands suppressive Tregs in humans with autoimmune disease, and in animal models of autoimmunity. TNFR2 signaling promotes Treg cell expansion and suppressive activity in experimental autoimmune encephalomyelitis (EAE; an animal model of inflammatory CNS demyelinating disease, e.g., multiple sclerosis), and in a murine model of diabetes, and induces human antigen-specific Treg cells by tolerogenic dendritic cells. TNFR2-deficient Tregs are reduced in their ability to prevent experimental colitis in vivo, and TNFR2 is required for sustained FoxP3 expression on Tregs, and as a result, for maintaining the phenotypic and functional stability of Tregs, indicating that TNFR2 is required for the in vivo immunosuppressive function of Tregs (see, e.g., McCann et al. (2014) Arthritis & Rheumatology 66(10):2728-2738; Faustman and Davis (2013) Front. Immunol. 4:478; Schmidt et al. (2013) Arthritis & Rheumatism 65(9):2262-2273; Vanamee et al. (2017) Trends in Molecular Medicine 23(11):P1037-P1046; Chen et al. (2013) J. Immunol. 190(3):1076-1084). In one study, in vitro produced antigen-specific Tregs were shown to suppress disease and reduce joint inflammation and bone destruction in a well-established antigen-induced arthritis (AIA) model, in which mice are immunized with methylated bovine serum albumin (mBSA) to induce T cell-mediated tissue damage (see, e.g., Wright et al. (2009) Proc. Natl. Acad. Sci. USA 106(45):19078-19083). Using Tregs in cellular therapy, while promising, due to manufacturing and other complications, a traditional biologic therapeutic that provides the advantages of Tregs without the complications is needed.

As described and provided herein, TNFR2, and its expression by Tregs, is required for the suppression of inflammatory and autoimmune diseases and conditions. For example, the Mycobacterium bovis bacillus Calmetter-Guerin (BCG) induces transient expansion of Tregs. In a clinical trial, BCG triggered Treg production in patients with type I diabetes, resulting in suppression of disease and temporary restoration of islet cell function, indicating a use of Tregs and/or modulators that enhance Treg function in the treatment of type I diabetes (see, e.g., Spence et al. (2016) Curr Diab Rep 16(11):110. doi: 10.1007/s11892-016-0807-6).

It is described and established herein that modulation of Treg function presents a therapeutic approach for the prevention or treatment of inflammatory and autoimmune diseases and conditions. Tregs, however, only constitute~1-5% of total CD4⁺ T cells in the blood. Their low numbers hinder their clinical use. Ex vivo generation of Tregs, and/or stimulation of their production in vivo, is factor that limits their therapeutic use. For example, in vivo stimulation with IL-2, anti-CD3, or anti-CD28 is too toxic, while ex vivo stimulation using these agents generates heterogeneous CD4⁺populations that can release proinflammatory cytokines and have antagonistic properties. Alternative approaches have used TL1A-Ig, a naturally occurring TNF receptor superfamily agonist, or TNFR2 monoclonal antibody agonists, to expand Tregs in vivo and ex vivo, respectively. A TNFR2 agonist construct, and the multi-specific constructs, provided herein can preserve and/or expand the Treg population in vivo without interfering with the therapeutic activity of anti-TNFR1 activity. As described and provided herein, selective inhibition of inflammatory TNFR1 activity, while maintaining or increasing TNFR2-associated Treg suppressive activity, is beneficial in the treatment of inflammatory and autoimmune diseases and conditions. These diseases and conditions include, but are not limited to, RA, type I diabetes, heart failure and multiple sclerosis (see, e.g., Goodall et al. (2015) PLoS ONE 10(9):e0137065).

In a tumor microenvironment (TME), in contrast to an autoimmune microenvironment in which the expansion of TNFR2⁺Tregs prevents tissue destruction, tumors are infiltrated by large numbers of immunosuppressive TNFR2⁺Tregs, which prevent the proliferation of tumor-killing CD8⁺cytotoxic T lymphocytes (CTLs), also known as effector T cells (Teffs), allowing for tumor growth. Antagonism of TNFR2 on lymphocytes in the TME restores the balance between the two types of T cells, by inhibiting or eliminating Tregs and allowing for the activation and expansion of effector T cells, a condition where tumor growth can be controlled or reversed. To be useful as a therapeutic, the TNFR2 inhibitor must not have the ability to aggregate immune cells via ADCC for two reasons: 1) aggregation transiently leads to ‘super-induction’ of TNFR2 mediated immunosuppression; and 2) eventually leads to systemic depletion of Tregs, which will be detrimental to the patient because it is essential to retain a basal level of Treg activity to maintain immune homeostasis. Tumor cells and myeloid-derived suppressor cells (MDSCs) also express TNFR2, and inhibition of TNFR2 in MDSCs control metastasis, as shown in a murine liver cancer model. Thus, blockade of TNFR2, such as through the use of non-aggregating antagonistic antibodies or other therapeutics, as provided herein, presents a useful treatment for certain types of cancers via the inhibition of immunosuppressive Tregs. TNFR2 antagonists, however, only should be administered to patients whose tumors show overexpression of TNFR2 compared to adjacent normal tissue as judged from immunohistochemistry. Thus, such treatment should be accompanied by diagnostics to confirm overexpression (see e.g., Zhang et al. (2019) Thorac Cancer 10(3):437-444. doi:10.1111/1759-7714.12948; Yang et al. (2017) Oncol Lett.14(2):2393-2398. doi:10.3892/ol.2017.6410; and Yang et al. (2018) Oncol Lett. 16(3):2971-2978. doi:10.3892/ol.2018.8998, for exemplary assays).

4. Autoimmune/Inflammatory Diseases Mediated by or involving TNF

Elevated levels or uncontrolled expression of TNF and deregulation of TNF signaling can cause chronic inflammation, which can result in the development of autoimmune diseases and tissue damage. TNF-α is involved in numerous diseases, disorders, and conditions. Constructs provided herein can be used for treatment of such diseases, disorders, and conditions. The following discussion describes some exemplary diseases, disorders, and conditions in which blocking TNF can have a therapeutic effect. TNF blockers, such as etanercept, infliximab, adalimumab, certolizumab and Golimumab, have adverse side effects that can limit their use for treatment of such diseases, disorders, and conditions. The constructs provided herein, which avoid some or all of these adverse effects, can be used to treat these diseases, disorders, and conditions (see, e.g., Lis et al. (2014) Arch Med Sci. 10(6):1175-1185 for a review of the role of TNF in disease and the use of TNF blockers for treatment thereof).

Inflammatory diseases include an array of disorders and conditions that are characterized by inflammation, and include autoimmune diseases. The immune system protects the body by producing antibodies and/or activating lymphocytes in response to invading microorganisms, such as viruses and bacteria. In healthy individuals, the immune system does not trigger a response against the body's own (i.e., “self”) cells; autoimmune diseases occur when the immune system attacks healthy, non-invading, self, cells and tissues. Autoimmune/inflammatory diseases and disorders associated with elevated TNF levels include, for example, arthritis (e.g., rheumatoid arthritis, psoriatic arthritis, juvenile idiopathic arthritis, spondyloarthritis), inflammatory bowel disease (e.g., Crohn's disease and ulcerative colitis), uveitis, fibrotic diseases, endometriosis, lupus, ankylosing spondylitis, psoriasis, multiple sclerosis (MS), Parkinson's disease, and Alzheimer's disease, among others. Exemplary autoimmune and inflammatory diseases and disorders, that can be treated with the constructs provided herein, are discussed below.

a. Arthritis

### Rheumatoid Arthritis and Other Types of Arthritis

Rheumatoid arthritis (RA) is a chronic autoimmune inflammatory disease. The inflammation associated with rheumatoid arthritis affects the linings of the joints (i.e., the synovial lining), and also the membranes lining the blood vessels, heart and also can become inflamed. RA is characterized by the infiltration of immune cells (e.g., activated B cells) into the synovial membrane and synovial cell proliferation, which results in the thickening of the synovial lining. The proliferative mass, known as the pannus, invades and destroys cartilage and bone, irreversibly destroying joint structure and function. This is mediated by the induction of proinflammatory cytokines, such as TNF, IL-1 and IL-6. Tumor necrosis factor a (TNF-α) is a key modulator of the induction and perpetuation of the proinflammatory activities that are associated with RA. TNF is over-expressed in synovial fluids and in the synovial membrane, and expression of TNFRs is up-regulated in the synovial membrane (see, e.g., Blüml et al. (2012) International Immunology 24(5):275-281; Schmidt et al. (2013) Arthritis & Rheumatism 65(9):2262-2273; Keffer et al. (1991) EMBO J. 10(13):4025-4031). Other types of arthritis that can be treated with constructs herein, include, for example, psoriatic arthritis, juvenile idiopathic arthritis, and spondyloarthritis.

b. Inflammatory Bowel Disease (IBD) and Uveitis

Inflammatory bowel disease (IBD) includes Crohn's disease and ulcerative colitis, which are inflammatory diseases of the intestine and colon. Mice overexpressing TNF develop intestinal inflammation that resembles Crohn's disease, while TNFR1 deficiency protects against Crohn's disease (see, e.g., Fischer et al. (2015) Antibodies 4:48-70).

Uveitis is a form of eye inflammation that affects the eye wall (uvea), the middle layer of the eye between the retina and the sclera (white of the eye), and can lead to vision loss. TNF-α is involved in its pathophysiology, and TNF blockers have been used for treatment.

c. Fibrotic Diseases

Constructs herein can be used for treatment of fibrotic diseases. Dupuytren's disease is exemplary of such diseases. Dupuytren's disease (DD) is a common fibrotic condition of the hands that is characterized by irreversible flexion contractures of the fingers; the condition is limited to the palm of the hand and causes irreversible curling in of the fingers, severely compromising hand function. There are no approved therapies for early stage disease, which manifests as nodules that are quiescent for some time and that then become active and progress to cords and flexion deformities of the fingers, resulting in the loss of hand function. Treatment involves surgical excision (fasciotomy) of the diseased tissue or cords, or disruption of the cords using collagenase or needle fasciotomy. The surgical and non-surgical treatments have high rates of recurrence and complications. Therapeutic intervention at the early stages of disease, to prevent progression to cord development and the subsequent flexion contractures of the digits, is advantageous (see, e.g., Nanchahal et al. (2018) EBioMedicine 33:282-288).

Myofibroblasts, which express the contractile protein a-smooth muscle actin (α-SMA) and aggregate in nodules, deposit excessive collagenous extracellular matrix and are responsible for its remodeling and contraction in all fibrotic conditions, including DD. TNF converts palmar fibroblasts into myofibroblasts in patients with DD, via the Wnt signaling pathway, and DD myofibroblasts exhibit a dose-dependent reduction in contractility and reduction in the expression of a-SMA and pro-collagen, following treatment with anti-TNF therapies. Treatment with the fully humanized IgG mAbs adalimumab and golimumab have been the most effective. The use of anti-TNF therapies, such as adalimumab, however, is associated with an increased risk of infection, and in a phase 2a trial evaluating the therapeutic efficacy of adalimumab in DD, 1 patient (out of 21 receiving adalimumab) developed a wound infection requiring hospitalization (see, e.g., Nanchahal et al. (2018) EBioMedicine 33:282-288). Thus, other therapies are needed.

d. Tumor Necrosis Factor Receptor-Associated Periodic Syndrome (TRAPS)

Tumor necrosis factor receptor-associated periodic syndrome (TRAPS) is the second most common inherited autosomal dominant auto-inflammatory disease, and is caused by mutations in the TNFRSF1A gene, encoding TNFR1. TRAPS is characterized by unprovoked, periodic long-lasting fever, systemic inflammation, abdominal pain, skin lesions, conjunctivitis, myalgia and pericarditis, with inflammatory attacks lasting up to several weeks. A complication associated with more severe clinical phenotypes of TRAPS is AA-type serum amyloidosis, which can result in renal impairment and failure. Disease onset typically occurs in early childhood, but TRAPS can present in adults as well. The majority of TRAPS-associated mutations occur in the extracellular domain of TNFR1, which is involved in ligand binding. High-penetrance mutations, which are associated with the most severe clinical phenotype, occur in the extracellular cysteine-rich domains (CRDs). The mutations affect the folding and secondary structure of TNFR1, which can result in defective TNFR1 trafficking, altered ligand binding affinity, reduced activation-induced shedding and impaired cell signaling. For example, ligand-independent gain-of-function of TNFR1 induces TRAPS pathophysiology, and certain mutations result in the constitutive activity of TNFR1, NF-κB and caspase 1. Traditional anti-TNF therapies, including etanercept, infliximab, and others, are only partially effective in the treatment of TRAPS (see, e.g., Greco et al. (2015) Arthritis Research & Therapy 17:93), and thus, other therapies are needed.

e. Other Diseases Mediated by or Involving TNF
i. Neurodegenerative Diseases

Aging and several neurodegenerative diseases are associated with elevated levels of TNF in the central nervous system (CNS). TNF is implicated in initiating and maintaining neuroinflammation, and in modulating other neurological processes, such as synaptic function and plasticity. The levels of TNFR1 in the hippocampus of aged rats is approximately 3-fold higher compared to the levels of TNFR2. In animal models of disease, TNF is implicated in chronic glial activation and impaired neuronal viability through its actions on TNFR1. In aged animals, neurologic changes include synaptic dysfunction and Ca²⁺ dysregulation, which can be replicated in healthy young animals and in neuronal cultures using artificial elevations in TNF. TNF also potentiates the activity of L-type voltage sensitive Ca²⁺ channels (L-VSCCs); a similar effect is observed in hippocampal neurons of memory impaired aged rats. Studies in rats have shown that TNF blockade in the cerebellum accelerates learning in a delayed eyeblink task. Selective blockade of TNFR1 signaling, using XPro1595, a soluble dominant negative TNF (DN-TNF) that preferentially inhibits TNFR1 signaling, resulted in improved behavioral performance on a Morris swim task, reduced microglial activation, prevention of hippocampal long-term depression (LTD), and reduced the activity of L-VSCCs in CA1 neurons. These results indicate that TNF signaling via TNFR1 is implicated in modifying the neurologic phenotype of aged animals, and can result in pathological changes associated with neurological diseases (see, e.g., Sama et al. (2012) PLoS ONE 7(5):e38170).

### a) Alzheimer's Disease

TNF is a central player in inflammatory responses; TNF protein levels are low in healthy brain but chronically elevated in many neuroinflammatory diseases, including Alzheimer's disease (AD). In animal models of AD, TNF promotes microglial activation, synaptic dysfunction, neuronal cell death, accumulation of plaques and tangles, and cognitive decline. For example, in a triple transgenic AD mouse model (3×Tg-Ad), with mutations in presenilin 1, amyloid precursor protein (APP) and tau, TNF levels were elevated in entorhinal cortex, coincident with the earliest appearance of pathology (see, e.g., McCoy et al. (2006) J. Neurosci. 26(37):9365-9375). TNF-driven processes are implicated in AD pathology and contribute to cognitive dysfunction and accelerated progression of AD. The bacterial endotoxin lipopolysaccharide (LPS), which induces inflammation and the production of TNF, accelerates the appearance and severity of AD pathology in several animal models of AD. The overproduction of proinflammatory mediators, including TNF, occurs in the brain when microglia, which are often in close physical association with amyloid plaques in AD brains, become chronically activated. Elevated levels of TNF inhibit phagocytosis of amyloid beta (Aβ) in the brains of AD patients, which hinders efficient plaque removal by microglia. The chronic inhibition of solTNF by administering a DN-TNF, such as XENP345, or a lentivirus encoding the DN-TNF, prevented the acceleration of AD-like pathology induced by chronic systemic inflammation in an animal model of AD (3×TgAD mice), and decreased the LPS-induced intraneuronal accumulation of 6E10-immunoreactive protein, particularly C-terminal amyloid precursor protein (APP) fragments (β-CTF), in the hippocampus, cortex and amygdala. Genetic deletion of TNFR1 in 3×TgAD mice also prevents the LPS-induced accumulation of 3-CTF, which is neurotoxic. Neuronal cells bearing familial AD (FAD) mutations accumulate 3-CTF intracellularly, implicating its involvement in the pathogenesis of AD. These results indicate that soluble TNF is a mediator of the effects of neuroinflammation on early, pre-plaque pathology in 3×TgAD mice, and that targeted inhibition of solTNF in the central nervous system (CNS) can slow the appearance of amyloid-associated pathology, cognitive deficits, and the progressive loss of neurons in AD (see, e.g., McAlpine et al. (2009) Neurobiol. Dis. 34(1):163-177).

### b) Parkinson's Disease

Parkinson's disease (PD) is the second most prevalent neurodegenerative disease in the United States, with an incidence of 5% in individuals over 65 years of age. The clinical manifestations of Parkinson's disease result from the selective loss of dopaminergic neurons in the ventral mesencephalon substantia nigra pars compacta (SNpc), which results in a decrease in striatal dopamine. The cerebrospinal fluid (CSF) and postmortem brains of patients with PD and animal models of PD show elevated levels of TNF. A cohort of early-onset PD patients in Japan showed an increased frequency of a polymorphic allele (-1031C) in the TNF gene promoter that results in higher transcriptional activity and elevated TNF levels. TNFR1 is highly expressed in nigrostriatal dopaminergic neurons, which increases vulnerability to TNF-induced neuroinflammation and dopaminergic neuron toxicity. The in vivo neutralization of soluble TNF (solTNF) by a dominant-negative TNF mutein (XENP345) was neuroprotective, and reduced the retrograde nigral degeneration induced by a striatal injection of the oxidative neurotoxin 6-hydroxydopamine (6-OHDA) by 50% and attenuated amphetamine-induced rotational behavior in rats, indicating preservation of striatal dopamine levels. Delayed administration of XENP345 in embryonic rat midbrain neuron/glia cell cultures exposed to lipopolysaccharide (LPS) prevented the degeneration of dopaminergic neurons, despite sustained microglia activation and secretion of solTNF. XENP345 also attenuated 6-OHDA-induced dopaminergic neuron toxicity in vitro. TNF, thus, is implicated in the development of Parkinson's disease, and it may be possible to delay the progressive degeneration of the nigrostriatal pathway in humans by using TNF-blocking therapeutics, particularly in the early stages of Parkinson's disease (see, e.g., McCoy et al. (2006) J. Neurosci. 26(37):9365-9375).

### c) Multiple Sclerosis (MS)

CNS-specific overexpression of TNF in transgenic mice results in spontaneous demyelination, which is indicative of a role of TNF in multiple sclerosis (MS). A polymorphism in the gene encoding TNFR1 is linked to an increased susceptibility of developing MS. TNFR1 is necessary for the disease induction of experimental autoimmune encephalomyelitis (EAE), an animal model of MS, and TNFR2 deficiency worsens the disease. Mice expressing non-cleavable membrane-bound TNF are protected against EAE, indicating that the interaction of soluble TNF with TNFR1 is associated with disease pathology (see, e.g., Fischer et al. (2015) Antibodies 4:48-70).

ii. Endometriosis

TNF-α has been implicated in the pathophysiology of endometriosis. TNF-α levels are increased in peritoneal fluid of women with endometriosis, and the levels correlate with severity of disease (see, e.g., Koninckx (2008) Hum Reprod. 23: 2017-2023). Peritoneal fluid TNF-α is produced locally by activated peritoneal macrophages, and TNF-α induces IL-8 secretion by peritoneal mesothelial cells. The peritoneal fluid concentrations of TNF-α and IL-8 correlate with the size and the number of active peritoneal lesions (Bullimore, (2003) Med Hypotheses. 60:84-88). Serum TNF-α levels are increased, and monocytes from patients with endometriosis release more TNF-α in vitro compared with monocytes from controls. Peritoneal fluid levels of MCP-1 are increased in patients with endometriosis. TNF-α, IL-8 and MCP-1 drive an inflammatory Th-1 type response in the peritoneal fluid of patients with endometriosis. TNF-α mediated inflammation may be a causal factor in the pain associated with endometriosis. Blocking TNF-α appears to inhibit the development of the disease in animal models, and may be effective for humans. Because of the adverse side effects of existing TNF blockers, treatment of endometriosis with such blockers has not been recommended (see, Koninckx (2008) Hum Reprod. 23: 2017-2023). Constructs provided herein, however, are designed to avoid the deleterious effects, and can be considered for treating TNF-α mediated inflammation in endometriosis.

iii. Cardiovascular Disease

TNF-α was the first cytokine to be identified in human atherosclerotic plaque; TNF-α is involved in the activation of the endothelium and upregulation of adhesion molecules, which occur early in the development of atherosclerotic disease. TNF also is implicated in the pathogenesis of atherosclerosis by affecting lipid metabolism and inducing vascular inflammation. The blockade of TNF-α by TNF binding protein, or IL-1 by an IL-1 receptor antagonist, partially protects apoE knockout mice from atherosclerosis. Atherogenesis primarily is the result of the production of TNF-α by myeloid cells. The plaque area in apoE^{-/-} and TNF^{-/-} mice on a high fat diet is half the size of the plaque area in mice that are apoE^{-/-}. Transplantation of bone marrow from apoE^{-/-} and TNF^{-/-} mice, into apoE^{-/-} mice, reduced atherosclerotic lesion size by 83%. Atherosclerotic lesion size also was reduced following treatment of apoE^{-/-} mice with a recombinant soluble p55 (TNFR1) TNF blocker, indicating the role that TNF plays in atherosclerosis. NF-κB signaling is involved in the production of TNF-α in human atherosclerotic plaques. The peripheral blood levels of TNF-α in patients with cardiovascular disease also is correlated with the development of myocardial infarction. Cardiotoxicity primarily is attributed to TNF-induced cardiomyocyte apoptosis. The use of anti-TNF therapies, such as infliximab and etanercept, in clinical trials for the treatment of heart failure failed, and resulted in increased mortality; anti-TNF therapies have thus not been tested for the treatment of cardiovascular disease (see, e.g., Udalova et al. (2016) Microbial Spectrum 4(4):MCHD-0022-2015; Kalliolias and Ivashkiv (2016) Nat. Rev. Rheumatol. 12(1):49-62). As a result, alternative therapies are required.

iv. Acute Respiratory Distress Syndrome (ARDS)

Acute respiratory distress syndrome (ARDS) affects approximately 190,000 patients per year in the U.S. and has mortality rates of up to 40%. There are no effective therapeutics targeting the underlying pathophysiological mechanisms of ARDS. ARDS is characterized by immune cell-mediated lung injury, which is associated with the release of inflammatory cytokines and proteases. The uncontrolled local inflammatory response in ARDS results in damage to the alveolar-capillary barrier, and non-cardiogenic pulmonary edema. Pulmonary neutrophil recruitment, which is central to the pathogenesis of ARDS, is mediated by the interaction of primed and activated neutrophils with the lung microvascular endothelium, and is increased by damage to the alveolar-capillary barrier caused by the action of proinflammatory mediators. TNF-α has been identified as a key effector molecule in ARDS, as well as in sepsis, which is a common cause of ARDS. For example, TNF-α contributes to increased endothelial permeability. Clinical trials involving the administration of non-selective anti-TNF antibodies for the treatment of sepsis have failed to demonstrate any survival benefit, and one trial indicated that higher doses were harmful.

TNFR1-deficient mice are protected from lung injury, sepsis and other acute organ injuries, while TNFR2-deficient mice are more susceptible to injury in these models, indicating that selective antagonism of TNFR1 can be therapeutically effective. GSK1995057, a short-acting, fully human domain antibody (dAb) fragment that selectively antagonizes TNFR1, but not TNFR2, attenuated disease severity in a murine model of acute respiratory distress syndrome, and attenuated inflammation and signs of lung injury in non-human primates. In a randomized, placebo-controlled trial of nebulized GSK1995057 in 37 healthy humans challenged with a low dose of inhaled endotoxin, treatment with GSK1995057 attenuated pulmonary neutrophilia, inflammatory cytokine release, and signs of endothelial injury in bronchoalveolar lavage and serum samples. These results indicate the potential for pulmonary delivery of selective TNFR1 antagonists for the prevention and treatment of ARDS (see, e.g., Proudfoot et al. (2018) Thorax 73:723-730).

v. Severe Acute Respiratory Syndrome (SARS) and COVID-19

Subjects infected with severe acute respiratory syndrome coronavirus (SARS-CoV) present with fever and respiratory illness, general malaise and lower respiratory tract symptoms, including cough and shortness of breath, with an overall fatality rate of ~10%. TNF signaling promotes pathogenesis of SARS, by inducing excessive inflammation, which causes significant tissue damage. The development of a cytokine release syndrome (CRS) plays a role in severe COVID-19, the disease caused by severe acute respiratory syndrome coronavirus 2 (SARS-CoV-2). The persistent viral stimulation results, in some subjects in an increase in the levels of circulating cytokines, such as IL-6 and TNF-α, which leads to reduced lymphocyte counts and triggers inflammatory organ damage, particularly to the lungs, and blood vessels. SARS-CoV-2 shares several similarities with SARS-CoV, the strain of coronavirus responsible for the SARS pandemic of 2002. SARS-CoV and SARS-CoV-2 use the spike (S)-proteins to engage their cellular receptor, ACE2 (angiotensin-converting enzyme 2), for invading cells. The expression of the ACE2 receptor is upregulated by SARS-CoV-2 infection and by inflammatory cytokine stimulation. In SARS-CoV infection, S-proteins induce the TNF-α-converting enzyme (TACE)-dependent shedding of the ACE2 ectodomain, which is a process that is strictly coupled to TNF-a production. The loss of ACE2 activity due to shedding is associated with lung injury due to an increased activity of the renin-angiotensin system. ACE2 knockout mice are susceptible to severe respiratory failure following chemical challenge, and ACE2 has been shown to moderate ACE-induced intracellular inflammation. ACE2 downregulation is linked to the severe respiratory distress associated with SARS-CoV infection. Increased TNF-α production can thus facilitate viral infection and result in organ damage, such as lung injury.

As discussed, regulatory T cells (Tregs) are a type of immunosuppressive cell that display diverse clinical applications in transplantation, allergy, infectious disease, GVHD, autoimmunity, and cancer. Tregs co-express CD4⁺ and the interleukin-2 receptor alpha chain CD25^{hi} and feature inducible levels of intracellular transcription factor forkhead box P3 (FOXP3). Naturally-occurring Tregs express TNFR2 at a higher density than TNFR1. TNF signaling through TNFR2 promotes Treg activity: TNF-mediated TNFR2 activates and induces proliferation of Tregs (100) and TNFR2 expression indicates maximally suppressive Tregs Thus, in the case when TNF is being overproduced in response to an infection (influenzas, SARS type viruses, endotoxemia) Treg's can prevent overreaction to inflammatory stimuli.

Anti-TNF can be a treatment for SARS and COVID-19. Adalimumab is being used for treatment of COVID-19 (clinical trial China in February, 2020 (ChiCTR2000030089); see, e.g., Lucchino et al. (2020) Rheumatology (Oxford) 59(6):1200-1203; Haga et al. (2008) Proc. Natl. Acad. Sci. U.S.A. 105:7809-7814). Knockout of TNFR2 in mice infected with SARS-CoV does not provide any protective effects; the double knockout of TNFR1 and TNFR2 protected infected mice from weight loss associated with infection (see, e.g., McDermott et al. (2016) BMC Systems Biology 10:93). These results indicate that TNF signaling through TNFR1 primarily contributes to the pathogenesis of SARS-CoV infection, by increasing proinflammatory processes, and that selective inhibition of TNFR1, rather than inhibition of TNF, is a better therapeutic approach. The constructs provided herein can be used to treat the acute inflammatory aspects of SARS and COVID-19. The constructs are used in combination with anti-infective agents; the constructs are used to suppress or ameliorate the acute effects of cytokine storm.

TNF-α inhibition reduces the severity of virally-induced lung diseases, such as those caused by respiratory syncytial virus (RSV) or influenza virus, in mice. The depletion of TNF using anti-TNF antibody in these mouse models reduced the pulmonary recruitment of inflammatory cells, reduced the production of proinflammatory cytokines (e.g., IFNγ, IL-4, IL-5, TNF) by T-cells, and reduced the severity of illness without interfering with viral clearance (see, e.g., Hussell et al. (2001) Eur. J. Immunol. 31:2566-2573). These results indicate that TNF inhibitors and TNF receptor antagonists can be beneficial in the treatment of human viral lung diseases, such as those caused by SARS-CoV and SARS-CoV2, by preventing or reducing TNF-induced immune activation and pulmonary injury.

Allogeneic hematopoietic stem cell transplantation is complicated by the development of non-infectious idiopathic pneumonia syndrome (IPS), an acute pulmonary dysfunction that resembles SARS pneumonia. Elevated levels of TNF-α have been found in the sera of patients who developed lung injury after allogeneic stem cell transplantation (SCT), and it has been shown that donor-derived alloreactive T-cells are associated with this process. In humans, anti-TNF therapy with etanercept is beneficial in the treatment of IPS after allogeneic stem cell transplantation. Recipients of allogeneic stem cell transplants are at high risk of developing bacterial and fungal infections, due to the immunoablative effects of SCT conditioning regimens, the requirement for long term use of immunosuppressive drugs to prevent or treat graft-vs-host disease (GvHD), and other SCT complications (including acute GvHD) that can impair host defenses (see, e.g., Yanik et al. (2002) Biol. Blood Marrow Transplant. 8:395-400). Other indications that can be treated by constructs provided herein include chemo brain, a condition experienced during and following chemotherapy, particularly women treated for breast cancer. Also, the treatments and constructs herein can be used to treat long COVID.

As a result, the use of selective TNFR1 antagonists, which preserves protective TNF signaling via TNFR2, and, unlike anti-TNF therapies, does not increase the risk of serious infections, provides a safer and more effective therapeutic option for the treatment, prevention or amelioration of virally- and non-virally-induced lung injury. The constructs provided herein, thus, are ideal therapeutics for these indications.

### D. Therapies for Rheumatoid Arthritis and Other Chronic Inflammatory and Autoimmune Diseases and Disorders

There is no cure for rheumatoid arthritis (RA), but treatments can improve symptoms and slow disease progression, for example, by minimizing pain and swelling, preventing bone deformity, and maintaining day-to-day functioning. The primary treatments for RA are disease-modifying anti-rheumatic drugs (DMARDs), which also are used for the treatment of other chronic inflammatory and autoimmune diseases and disorders, such as, for example, psoriasis, plaque psoriasis, psoriatic arthritis, juvenile idiopathic arthritis, ankylosing spondylitis, Behget's disease, inflammatory bowel disease (IBD; e.g., Crohn's disease and ulcerative colitis), multiple sclerosis, and lupus, as well as for the treatment of some cancers.

DMARDs are immunosuppressive and immunomodulatory agents that are classified as either conventional synthetic DMARDs (csDMARDs), or biological DMARDs (bDMARDs; e.g., antibodies and fusion proteins). Conventional synthetic DMARDs include, for example, methotrexate (MTX), a chemotherapy agent and immunosuppressant; hydroxychloroquine (HCQ; Plaquenil®), an anti-malarial agent; sulfasalazine (Azulfidine®), an anti-inflammatory drug; and leflunomide (Arava®), an immunosuppressant that inhibits dihydroorotate dehydrogenase. Biologic DMARDs include, for example, abatacept (Orencia®), a fusion protein that prevents T cell activation and contains the Fc region of IgG1 fused to the extracellular domain of CTLA-4; anakinra (sold, for example, under the trademark Kineret®), a recombinant human IL-1 receptor antagonist; rituximab (sold under trademarks, including Rituxan®, Truxima®, MabThera®), a chimeric monoclonal antibody against CD20, which induces apoptosis in CD20⁺cells, such as B cells; tocilizumab (atlizumab, Actemra®, RoActemra®), a humanized monoclonal antibody against the IL-6 receptor (IL-6R); corticosteroids; tofacitinib (Xeljanz®), a small molecule inhibitor of Janus kinase (JAK), a protein tyrosine kinase involved in mediating cytokine signaling; and TNF-inhibitors/anti-TNF agents, such as, for example, certolizumab pegol (Cimzia®), infliximab (Remicade®), adalimumab (Humira®), golimumab (Simponi®), and etanercept (Enbrel®). Combination therapy, particularly of methotrexate with a biological DMARD, is more effective than either therapy alone. Combination therapies also can include multiple csDMARDs and multiple csDMARDs with one biological DMARD. Due to the risk of serious side effects, including serious infections, multiple biological DMARDs, particularly anti-TNF DMARDs, typically are not used for combination therapy methods.

The following sections describe existing therapies, and the problems associated with each, to highlight how the therapies provided herein solve the problems.

1. Conventional Synthetic Disease Modifying Anti-Rheumatic Drugs (csDMARDs)

Conventional synthetic Disease Modifying Anti-Rheumatic Drugs (csDMARDs) are typically the first line treatment for RA and other autoimmune and chronic inflammatory diseases and disorders. csDMARDS include drugs such as methotrexate, leflunomide, hydroxychloroquine, and sulfasalazine, Methotrexate is the most commonly used agent for initial treatment, and its mechanism of action involves stimulating the release of adenosine from fibroblasts, reducing neutrophil adhesion, inhibiting leukotriene B4 synthesis by neutrophils, inhibiting local IL-1 production, reducing levels of IL-6 and IL-8, suppressing cell-mediated immunity, and inhibiting synovial collagenase gene expression. Other conventional synthetic DMARDs act by inhibiting the proliferation of lymphocytes or causing lymphocyte dysfunction. For example, leflunomide inhibits dihydroorotate dehydrogenase, resulting in inhibition of pyrimidine synthesis, and blocking lymphocyte proliferation. Sulfasalazine mediates its anti-inflammatory effects by preventing oxidative, nitrative and nitrosative damage, and hydroxychloroquine is a mild immunomodulatory agent that inhibits intracellular toll-like receptor 9 (TLR9).

Hydroxychloroquine, which has the best safety profile of conventional DMARDs, does not increase the risk of infections, and does not cause hepatotoxicity or renal dysfunction; common side effects of hydroxychloroquine include rash and diarrhea. Retinopathy/maculopathy is a rare but serious side effect of hydroxychloroquine therapy which is associated with doses of more than 5 mg/kg/day, long-term use (more than 5 years of therapy), older age and chronic kidney disease. Other rare adverse effects of hydroxychloroquine include anemia, leukopenia, myopathy, and cardiomyopathy. Therapy with methotrexate, leflunomide, and sulfasalazine is associated with nausea, abdominal pain, diarrhea, rash/allergic reaction, bone marrow suppression, hepatotoxicity and higher incidence of common and sometimes serious infections. Methotrexate and leflunomide also cause alopecia. Other side effects associated with methotrexate therapy include interstitial lung disease, folic acid deficiency, and liver cirrhosis. Leflunomide also is associated with hypertension, peripheral neuropathy, and weight loss. Sulfasalazine has a very high risk of gastrointestinal distress and can rarely cause DRESS syndrome (drug reaction with eosinophilia and systemic symptoms) (see, e.g., Benjamin et al. Disease Modifying Anti-Rheumatic Drugs (DMARD) [Updated 2020 Feb. 27]. In: StatPearls [Internet]. Treasure Island (FL): StatPearls Publishing; 2020 January Available from: URL:ncbi.nlm.nih.gov/books/NBK507863/). These drugs are effective because they are immunosuppressive. The constructs provided herein that are selective anti-TNFR1 antagonists that preserve TNFR2 immunosuppressive activity advantageously can avoid the need for these immunosuppressive drugs.

### 2. Anti-TNF Therapies/TNF Blockers

Anti-TNF therapies/TNF-blockers (a type of biological DMARD) typically are prescribed after the failure of conventional DMARDs, and include monoclonal antibodies (mAbs), such as the chimeric mAb infliximab (Remicade®); containing a murine variable region and a human IgG1 constant region, and the fully humanized mAbs (IgG1s) adalimumab (Humira®) and golimumab (Simponi®); the PEGylated humanized Fab′ fragment of a mAb targeting TNF, certolizumab pegol (Cimzia®); and TNFR2 fusion proteins, such as the TNFR2-Fc fusion protein etanercept (Enbrel®), which contains the extracellular receptor region that contains the binding site of human TNFR2 fused to the Fc of human IgG1. Remsima® and Inflectra® are biosimilars of infliximab that are approved for use in the European Union for the treatment of various autoimmune and chronic inflammatory diseases and disorders. These TNF inhibitors, which sequester TNF, are used for the treatment of various diseases and conditions, including, for example, RA, psoriasis, psoriatic arthritis, ankylosing spondylitis, juvenile idiopathic arthritis (JIA) and/or inflammatory bowel disease (IBD; e.g., Crohn's disease and ulcerative colitis).

Because of the immunosuppressive effects of therapies that target TNF, such therapies are associated with severe side effects, including, for example, an increased risk of sepsis and serious infections, such as listeriosis, reactivation of tuberculosis, reactivation of hepatitis B/C, reactivation of herpes zoster, and invasive fungal and other opportunistic infections. TNF is a key cytokine in the inflammatory and immune responses to infections, and the use of drugs that remove TNF impairs host immunity against microorganisms, increasing the risk of infection. For example, TNF blocking agents are associated with the reactivation of M. tuberculosis infection. TNF plays an important role in the resistance against Mycobacterium tuberculosis, and adalimumab therapy in RA patients significantly reduces reactivity against M. tuberculosis. As described herein, the reduced immune reactivity can be related to the activation of Tregs and the induction of apoptosis in effector lymphocytes. Anti-TNF therapy has been shown to induce macrophage apoptosis in the rheumatoid synovium. Infliximab is associated with increased apoptosis in the inflammatory cell infiltrate in the guts of patients with Crohn's disease. Other anti-rheumatic drugs, such as methotrexate and glucocorticoids, also can induce apoptosis in immune cells (see, e.g., Vigna-Perez et al. (2005) Clin. Exp. Immunol. 141(2):372-380). Adalimumab and infliximab, but not etanercept, a TNFR2-Fc fusion protein, induce caspase-dependent apoptosis in cultured monocytes, and downregulate the production of IL-10 and IL-12 by monocytes (see, e.g., Shen et al. (2005) Ailment Pharmacol. Ther. 21:251-258). The most prevalent fungal infections associated with TNF blockers are histoplasmosis, candidiasis, and aspergillosis. Anti-TNF agents also can cause worsening of severe congestive heart failure, drug-induced lupus, and demyelinating central nervous system (CNS) diseases, as well as lymphomas and non-melanoma skin cancers (see, e.g., Benjamin et al. Disease Modifying Anti-Rheumatic Drugs (DMARD) [Updated 2020 Feb. 27]. In: StatPearls [Internet]. Treasure Island (FL): StatPearls Publishing; 2020 January Available from: ncbi.nlm.nih.gov/books/NBK507863/).

Infliximab also has been associated with the development of leukopenia, neutropenia, thrombocytopenia, and pancytopenia (some fatal). etanercept has been associated with an increased incidence of opportunistic bacterial and viral infections in patients with RA. etanercept also is used to treat severe refractory graft-versus-host disease (GvHD). Subjects with severe GvHD who are treated with etanercept have a very high risk (100% in one study, see, Zoran et al. (2019) Sci. Rep. 9:17231) of developing invasive aspergillosis (IA), a life-threatening mold (i.e., fungal) infection caused by Aspergillus fumigatus. Treatment with etanercept results in the downregulation of genes involved in immune responses and TNF signaling, including genes involved in NF-κB signaling, anti-microbial humoral responses and apoptotic processes, as well as a decrease in the secretion of chemokines, such as CXCL10, from immune cells (see, e.g., Zoran et al. (2019) Sci. Rep. 9:17231).

Other side effects associated with the use of TNF blocking therapies include congestive heart failure, liver injury, demyelinating disease/CNS disorders, lupus, psoriasis, sarcoidosis, and an increased susceptibility to the development of additional autoimmune diseases, as well as cancers, including lymphomas and solid malignancies (see, e.g., Dong et al. (2016) Proc. Natl. Acad. Sci. USA 113(43):12304-12309; Zalevsky et al. (2007) J. Immunol. 179:1872-1883; Zoran et al. (2019) Sci. Rep. 9:17231). Thus, the abrogation of all TNF-mediated signaling, by sequestering TNF, is not an ideal therapeutic strategy, as it results in severe immunosuppression that can lead to serious, sometimes fatal, infections, and other dangerous side effects.

Anti-TNF therapies ameliorate RA but are not curative, and require years of continuous and costly therapy. The inhibition/blockade of TNF in RA reduces inflammation and joint destruction, but, as discussed above, is associated with an increased risk of serious infections, such as tuberculosis and listeriosis, due to immunosuppression. As a result, the use of TNF blockers, particularly in the case of chronic diseases/conditions that require long-term administration, such as arthritis and IBD, is limited. Approximately 30% of RA patients are non-responsive, or therapeutic benefits are not sustained, with the use of anti-TNF therapies (see, e.g., McCann et al. (2014) Arthritis & Rheumatology 66(10):2728-2738). Non-responsiveness also occurs in non-RA patients receiving anti-TNF therapeutics. Depending on the anti-TNF agent, 13-33% of treated patients do not respond to treatment, and up to 46% stop responding, resulting in discontinuation or dose increase (see, e.g., Richter et al. (2019) MABS 11(4):653-665). Thus, there is a need for therapies with improved therapeutic efficacy and safety.

Anti-TNF therapeutics block/sequester TNF and inhibit soluble TNF (solTNF) and transmembrane TNF (tmTNF) signaling via TNFR1 and TNFR2, respectively; solTNF signaling has been associated with chronic inflammation, while tmTNF signaling has been associated with the resolution of inflammation and with the induction of immunity against pathogens such as Listeria monocytogenes and Mycobacterium tuberculosis. The primary anti-inflammatory effects of anti-TNF therapies are achieved by blocking TNFR1, while blocking TNFR2 inhibits Treg cell activity. As discussed elsewhere herein, TNFR1 signaling is primarily inflammatory and is involved in the pathogenesis of inflammatory and autoimmune diseases and conditions, such as RA, psoriasis, IBD, and neurodegenerative disorders, such as MS; whereas, TNFR2 signaling has anti-inflammatory and protective effects in various cell and organ types, including neural, cardiac, gut and bone tissues, and also is involved in host defense mechanisms against infection by pathogens. Thus, as described herein, selective blockade of TNFR1 improves the therapeutic efficacy in comparison to anti-TNF therapies, by eliminating undesirable, proinflammatory signaling associated with RA and other autoimmune and inflammatory diseases and conditions, while preserving the beneficial effects of TNFR2 signaling (see, e.g., McCann et al. (2014) Arthritis & Rheumatology 66(10):2728-2738; Schmidt et al. (2013) Arthritis & Rheumatism 65(9):2262-2273; Blüml et al. (2012) International Immunology 24(5):275-281; Zalevsky et al. (2007) J. Immunol. 179:1872-1883).

Anti-TNF therapies have failed in the treatment of neurodegenerative diseases, such as Alzheimer's disease, Parkinson's disease, stroke and multiple sclerosis (MS), which have been associated with the overexpression of TNF. For example, in a phase II trial for the treatment of relapsing remitting MS, a TNFR1 receptor-Fc IgG1 fusion protein anti-TNF therapeutic, lenercept (Ro 45-2081), failed, and symptoms were increased/worsened compared to patients receiving a placebo, with neurologic deficits being more severe in lenercept-treated patients. These results indicate that anti-TNF therapies can aggravate demyelinating diseases. While TNFR1 has been shown to mediate inflammatory neurodegeneration, TNFR2 induces neuroprotection, thus, blockade of signaling through both receptors by anti-TNF therapies abrogates the neuroprotective effects of TNFR2 signaling. The blockade of TNFR1 with ATROSAB, a humanized monoclonal antibody that blocks TNFR1, or the activation of TNFR2 with EHD2-scTNF_{R2}, an agonistic TNFR2-selective TNF mutein, results in the protection of cholinergic neurons against cell death, and reverts the neurodegeneration-associated memory impairment in a mouse model of NMDA-induced acute neurodegeneration. This is likely to be immunogenic. ATROSAB is a partial TNFR1 agonist; those of skill in the art would not administer a TNFR1 agonist. The blockade of TNFR1 and TNFR2, however, abrogates the therapeutic effect, indicating that TNFR2 plays an essential role in neuroprotection, and that selective blockade of TNFR1 can be used for the treatment of neurodegenerative diseases where anti-TNF therapies have failed (see, e.g., Dong et al. (2016) Proc. Natl. Acad. Sci. USA 113(43):12304-12309).

Due to the adverse effects associated with the use of anti-TNF agents, the non-responsiveness of some patients, the lack of a sustained response in patients that had an initial response, and the failure to treat and/or exacerbation of neurodegenerative diseases, such as MS, other therapies are needed. Such therapies are provided herein.

### E. Therapeutics for Targeting TNFR1/TNFR2

The following section discusses exemplary therapeutics that target TNFR1/TNFR2, and describes some problems and limitations with these therapeutics. Existing therapeutics can be modified as described in Section F and the Examples, or are used, in whole or in part, or are modified to improve their properties for use, in the constructs that are provided herein.

### 1. TNFR1-Selective Antagonists

As discussed and provided herein, therapy with TNF blockers, such as etanercept, infliximab, adalimumab, and others abrogates TNF signaling via TNFR1 and TNFR2. While TNFR1 signaling results in inflammation, cytotoxicity and apoptosis, TNFR2 signaling is protective and anti-inflammatory, partly due to its expansion and activation of immunosuppressive Tregs, which destroy effector T cells in the autoimmune environment, preventing tissue destruction and disease progression. Therapy with TNF blockers, through its inhibition of TNFR2 signaling, and the consequential depletion of immunosuppressive Tregs, which results in a pro-inflammatory microenvironment, can fail in the treatment of, and/or can exacerbate, autoimmune and inflammatory diseases and disorders. The dual blockade of TNFR1 and TNFR2 also can lead to opportunistic infections and cancer.

As provided herein, the specific inhibition of TNFR1 signaling maintains normal TNFR2 function, which is necessary for maintaining the equilibrium between pro-inflammatory and anti-inflammatory activity, via the production of subsets of both regulatory and cytotoxic T cells. Selective TNFR1 inhibition retains the potent anti-inflammatory activity of TNFR2 signaling, results in fewer opportunistic infections and cancer, and preserves TNF-induced Treg functions.

a. TNFR1 antagonistic Antibodies

Among TNFR1 antagonist antibodies, is ATROSAB (Antagonistic TNF Receptor One-Specific Antibody). ATROSAB, the first TNFR1 blocking antibody, is a full-length IgG1 that is a humanized version of the neutralizing mouse anti-human TNFR1 monoclonal antibody H398. It was abandoned as a therapeutic because it has partial agonist activity, which activates TNFR1, thereby mimicking TNF activity, a toxic pathway. ATROSAB maintains the conformation of TNFR1 in an inactive state and obstructs the binding of TNF. The Fc region in ATROSAB is mutated to eliminate FCγR receptor binding and complement fixation, thereby avoiding unwanted immune system activation (see, e.g., Kalliolias and Ivashkiv (2016) Nat. Rev. Rheumatol. 12(1):49-62).

Full-length antibodies have the advantage of increased in vivo half-life, but, as discussed elsewhere herein, are not feasible for the development of TNFR1 antagonists due to receptor cross-linking, which tends to agonize TNFR1 instead of antagonizing it. This did not result from Fc cross-linking because the Fc-interacting part of the antibody was removed by mutation. For example, the IgG ATROSAB exhibited some TNFR1 agonistic activity in the absence of TNF, which was observed to a limited extent at a narrow concentration range, due to its bivalent molecular structure. The cross-linking of TNFR1 also can occur due to secondary events, such as interactions with FcγRs or anti-drug antibodies (ADAs), which must be avoided to maintain the antagonistic nature of TNFR1 inhibitors. ADAs have been observed in patients treated with infliximab or adalimumab at rates of 50% and 31%, respectively (see, e.g., Richter et al. (2019) mAbs 11(4):653-665; Richter et al., (2019) mAbs 11(1):166-177).

For purposes herein, the TNFR1 antagonists, including in all embodiments described above and below are those that are selected to not have agonist activity and/or are designed or modified to eliminate any agonist activity. As described herein, such TNFR1 agonists include those that bind to the same epitope as the dAb of EN1206 (SEQ ID NO:59).

b. Monovalent TNFR1 antagonistic Antibodies/Antibody Fragments

Small antibody fragments, such as domain antibodies and derivatives and modified forms thereof have been developed, and exemplary antibody fragments and modified forms are discussed in the following sections. The small antibody fragments, however, have not been successfully developed into pharmaceuticals. They are limited in their use as therapeutics; they have short serum half-lives and fast peripheral clearance, which are a result of their small size. For example, molecules that are 50-60 kDa in size or smaller are subject to renal filtration; dAbs and other antibody fragments, which are less than 50-60 kDa in size, are rapidly cleared by the kidneys. For example, the dAb, designated DMS5541, and similar molecules demonstrate selectivity for TNFR1, and potentially can inhibit the deleterious effects of TNFR1 signaling. DMS5541, which is formed from two dAbs (antiTNFR1 and anti-human serum albumin), is only approximately 25 kDa in size, and is too small to have desirable pharmacokinetics for therapeutic purposes. Its association with HSA, which is meant to stabilize its half-life, is only 34 nM, which means it is often in a dissociated state with respect to HSA. Single-domain antibodies (sdAbs) that have been tested so far are expressed in E. coli, and are prone to aggregation (unfolding) during manufacturing. Additionally, sdAbs prepared cytoplasmically (from direct expression in E. coli) often lack the conserved disulfide bond found in variable heavy domains, which both decreases their melting point and can decrease their ability to refold. The rapid clearance and short elimination half-life of small antibody fragments, which can be less than a few hours, decreases the in vivo efficacy and necessitates frequent administration and/or continuous infusion, which can reduce patient compliance. Because these molecules (see, e.g., Holland et al. (2013) J Clin Immunol 33(7):1192-203) were produced in E. coli, and were often not correctly folded, leading to poor solubility and immunogenicity, resulting in their clinical failure (see, e.g., adisinsight.springer.com/drugs/800037882).

The constructs, such as the TNFR1 antagonist constructs, provided herein address this problem as well as other problems, such as the immunogenicity, and reactions with pre-existing antibodies. Provided herein are constructs containing small antibody fragments, such as dAbs, with specificity towards TNFR1 and/or TNFR2, that exhibit improved pharmacological, such as pharmacokinetic, properties, including longer serum half-life, increased stability, reduced/slower peripheral clearance, and lower immunogenicity compared to the dAbs of the prior art.

Therapeutic antibodies of a variety of structures can be potent and well-tolerated therapeutics. Antibodies are used for the treatment of a variety of diseases and conditions, including, for example, rheumatoid arthritis (e.g., adalimumab, sold under the trademark Humira®); cancers, such as non-Hodgkin's lymphoma (e.g., rituximab and ibritumomab tiuxetan, sold under the trademarks Rituxan® and Zevalin®, respectively) and breast and gastric cancers (e.g., trastuzumab, sold under the trademark Herceptin®); and respiratory syncytial virus infection (e.g., palivizumab, sold under the trademark Synagis®). Manufacturing of complete antibodies has several limitations, such as the reliance on mammalian cell expression. As a result, antigen-binding fragments of antibodies, such as Fabs (~57 kDa) and single chain Fv fragments (scFvs, ~27 kDa), and other structures, which can be selected in vitro, such as with phage display (circumventing animal immunization), and which can be manufactured in large quantities using bacterial or yeast cell cultures, have been developed. A Fab fragment contains a V_{H}-C_{H}1 polypeptide, linked to a V_{L}-C_{L} polypeptide via a disulfide bond; an scFv is a fusion protein containing a V_{H} domain and V_{L} domain linked by a short polypeptide linker. Another class of therapeutic, small fragments of antibodies are domain antibodies (dAbs; also known as single domain antibodies, or sdAbs), which are monomeric and contain a variable domain of the heavy chain (V_{H}) or of the light chain (V_{L}) of an antibody. dAbs are the smallest antigen-binding fragments of antibodies; they are approximately 11-15 kDa in size, which is about one-tenth the size of a full monoclonal antibody (mAb) (see, e.g., Holt et al. (2003) Trends in Biotechnology 21(11):484-490). Similar to dAbs, nanobodies (Nbs) are small antigen-binding fragments derived from camelid heavy-chain antibodies that are devoid of light chains. Nanobodies are small (~15 kDa), have low immunogenicity and high affinity, are soluble and stable, and are encoded by a single gene/exon (VHH), so that they are modular, which allows for high yield production in bacteria and yeasts (see, e.g., Steeland et al. (2015) J. Biol. Chem. 290(7):4022-4037; Steeland et al. (2017) Sci. Reports 7:13646).

i. Fab- and scFv-Based TNFR1 Antagonists

As discussed above, the humanized semi-agonistic/antagonistic TNFR1-specific antibody, ATROSAB, inhibits TNFR1-mediated cellular responses. ATROSAB exhibits some TNFR1 agonistic activity, likely due to its bivalent molecular structure or by virtue of its binding to TNFR1, in the absence of TNF. The parental mouse antibody, H398, possesses stronger inhibitory potential, which is due to the faster dissociation of ATROSAB (i.e., a higher k_{off} value) compared to H398. This was determined using quartz crystal microbalance (QCM) measurements, in which antigen density on the chip was reduced to favor monovalent interactions; a slower dissociation of monovalently bound H398 from TNFR1, and the resulting longer receptor occupation, contributes to the improved blockade of TNFR1. Thus, to eliminate the TNFR1 agonistic activity of ATROSAB, and to improve its TNFR1 antagonistic activity, monovalent derivatives of ATROSAB were developed.

To increase the affinity and antagonistic activity of ATROSAB, the single-chain variable fragment (scFv) of ATROSAB was subjected to a first affinity maturation by site-directed mutagenesis of exposed residues within individual CDRs, or combinations of CDRs, and selection by phage display against human TNFR1-Fc. The scFv of ATROSAB contains the V_{H} domain, corresponding to residues 1-115 of the ATROSAB heavy chain (see, SEQ ID NO:31), linked by a short peptide linker to the V_{L} domain, corresponding to residues 1-113 of the ATROSAB light chain (see, SEQ ID NO:32). A clone, scFv IG11 (see, SEQ ID NO:674), with 6 mutations within CDR-H2 of the ATROSAB heavy chain, Y52V, Y54T, S55Q, H57E, Y59K, and E62D, with reference to SEQ ID NO:31, exhibited slower receptor dissociation and improved equilibrium binding to human TNFR1-Fc, and improved inhibition of TNF-induced TNFR1 activation. This clone was further subjected to random mutagenesis, generating the clone scFv T12B (see, SEQ ID NO:675), containing the mutations Q1H, Y52V, Y54S, S55Q, H57E, Y59K, and E62D in the V_{H} domain (with reference to SEQ ID NO:31), and S96G in the V_{L} domain (with reference to SEQ ID NO:32). scFv T12B had reduced dissociation from immobilized TNFR1-Fc compared to the scFv of ATROSAB and to scFv IG11, and increased TNFR1 inhibitory activity (see, e.g., Richter et al. (2019) mAbs 11(1):166-177; see, also, Richter, F. Thesis, entitled “Evolution of the Antagonistic Tumor Necrosis Factor Receptor One-Specific Antibody ATROSAB,” Universitat Stuttgart, 2015; available from pdfs.semanticscholar.org/d8e7/8b87d76dce36225cid497939ef37445cfa8a.pdf).

The humanization of H398 was re-engineered by an exchange of VH and VL framework regions of H398 with alternative germline genes to optimize CDR arrangement. scFv 13.7, containing the VH domain of scFV T12B, linked by a short peptide linker to a newly humanized VL domain of H398, had similar binding to human TNFR1-Fc in ELISA and QCM, improved inhibition of TNF-induced TNFR1 activity, and improved thermal stability, with a 10 degree Celsius higher melting temperature compared to scFv T12B. Based on scFv 13.7, an IgG and a Fab, with constant regions identical to ATROSAB, were generated (IgG 13.7 and Fab 13.7, respectively), which had increased binding to TNFR1 compared to ATROSAB (1.4-fold) and the Fab of ATROSAB (Fab ATR; 8.7-fold), respectively. Fab 13.7 also had reduced dissociation from immobilized TNFR1-Fc, compared to Fab ATR, with an 18.8-fold improved monovalent affinity. Thus, affinity maturation and framework replacement resulted in improved binding to TNFR1 for Fab 13.7. Fab 13.7 and IgG 13.7 displayed selectivity towards TNFR1-Fc and did not bind to a TNFR2-Fc fusion protein; Fab 13.7 bound to human and rhesus TNFR1-Fc, but not to mouse and rat TNFR1-Fc, showing a similar binding pattern to ATROSAB. In vitro, monovalent Fab ATR and Fab 13.7 did not activate TNFR1, while ATROSAB displayed marginal activation of TNFR1 activity and IgG 13.7 strongly activated TNFR1. The agonistic activity of IgG 13.7 can be due to the improved affinity and slower dissociation from TNFR1, resulting in the formation of stable signaling competent receptor-antibody complexes. Fab 13.7 displayed improved inhibition of TNFR1 activity compared to Fab ATR and to ATROSAB, and lacked any agonistic activity. Incubation of Fab 13.7 or ATROSAB with cross-linking anti-human Fab serum, revealed that Fab 13.7 does not activate TNFR-1, while ATROSAB does (see, e.g., Richter et al. (2019) mAbs 11(1):166-177).

Compared to ATROSAB, which has an initial half-life of 0.44 hours, a terminal half-life of 32.1 hours, and an area under the curve (AUC) of 181 μg/ml×h, Fab 13.7 (with a molecular mass of ~47 kDa). Fab 13.7 displayed an initial half-life of 0.08 h, a terminal half-life of 1.4 h, and an AUC of 4.2 pg/ml x h, which were similar to the values obtained for Fab ATR. To extend the half-life, a Fab′ fragment, Fab′ 13.7, was generated by introducing a free cysteine residue at the C-terminus of the CH1 domain, which was chemically coupled to a branched PEG_{40kDa} moiety, generating Fab 13.7PEG. Fab 13.7 also was fused through its Fd and a short flexible linker to the N-terminus of mouse serum albumin (MSA), generating Fab 13.7-MSA. A monovalent Fab-Fc fusion protein was generated by fusing Fab 13.7 to a modified Fc, lacking the cysteine residues in the hinge region and the ability to dimerize via the CH3 domain, generating a one-armed half-IgG molecule (IgG1_{half}13.7). A monovalent Fv-Fc molecule also was generated by fusing the VH and VL domains to a hetero-dimerizing knob-into-hole (kih) Fc chain lacking the cysteine residues in the hinge region (Fv13.7-Fcₖᵢₕ). None of the derivatives showed any agonistic TNFR1 activity, and, compared to Fab 13.7, a slightly reduced binding to human TNFR1-Fc was observed for Fab13.7PEG, Fab13.7-MSA and IgG1_{half}13.7; binding of Fv13.7-Fcₖᵢₕ was not affected. Inhibition of TNF-mediated TNFR1 activity was reduced by 1.5-3.3 fold compared to Fab 13.7; Fab13.7PEG showed the strongest impairment in function, and Fv13.7-Fcₖᵢₕ showed the lowest change in bioactivity. IgG_{half}13.7 showed a similar half-life to Fab 13.7, and an AUC value that was increased by 7.1-fold. Fab13.7PEG, Fab13.7-MSA and Fv13.7-Fcₖᵢₕ had extended terminal half-lives, with values of 14.4 h, 9.7 h, and 10.5 h, respectively, and increased AUC values. Thus, the fusion protein Fv13.7-Fcₖᵢₕ, which was engineered for heterodimeric assembly of two peptide chains by using knobs-into-holes technology, displayed the best combination of improved pharmacokinetic properties and TNFR1 antagonistic activity (see, e.g., Richter et al. (2019) mAbs 11(1):166-177; see, also, Richter, F. Thesis, entitled “Evolution of the Antagonistic Tumor Necrosis Factor Receptor One-Specific Antibody ATROSAB,” Universitat Stuttgart, 2015; available from pdfs.semanticscholar.org/d8e7/8b87d76dce36225c1d497939ef37445cfa8a.pdf).

In another study, to improve the pharmacokinetic properties, such as serum half-life, of Fab 13.7, an IgG-like Fc was incorporated into the molecule, while retaining the Fab-like heterodimerization of the polypeptide chains. To achieve this, the variable domains of the heavy and light chains of Fab 13.7 were fused to the N-termini of newly generated heterodimerizing Fc chains, known as Fc-one/kappa (Fc1κ). The Fc heterodimerization approach is based on interspersed Ig domains, derived from the heterodimerizing IgG1 constant heavy chain domain, CH1, and the kappa light chain constant domain, CLκ, and containing sections of the IgG1 CH3 sequence to mediate FcRn binding and enable FcRn-mediated drug recycling in vivo. The interspersed Ig domains include “CH31,” which contains amino acid sequence fragments of CH1 and CH3, and “CH3kappa” (CH3κ), which contains amino acid sequence fragments of CLκ and CH3. IgG1 CH2 domains also were fused to the N-termini of the CH31 and CH3κ domains, to include the entire FcRn binding region of the IgG molecule. Addition of the IgG1 hinge region to the N-termini of the CH2 domains results in a covalently linked heterodimerizing Fc moiety, known as Fc1κ. In contrast to other Fc heterodimerization technologies, such as knobs-into-holes, which involve replacement of one or more amino acids at the CH3-CH3 interface, Fc heterodimerization was achieved by exchanging larger amino acid sequence stretches obtained from human antibody sequences. Asymmetric scFv-Fc1κ fusion proteins were prepared and compared to scFv fusions with Fcs containing knobs-into-holes, and heterodimer formation was similar or improved, compared to the fusions containing knobs-into-holes technology (see, e.g., Richter et al. (2019) mAbs 11(4):653-665).

The variable domains of the TNFR1-specific Fab 13.7 molecule were fused to the CH2 domain N-termini of the CH31- or CH3κ-containing Fc chains with a short peptide linker, by fusing the VH to the CH2-CH3κ chain and the VL to the CH2-CH31 chain (VL13.7-CH2-CH31/VH13.7-CH2-CH3κ; VL1C/VHκC), generating the monovalent TNFR1-specific antagonistic antibody-derived molecule (Fv-Fc1κ fusion protein), known as Atrosimab (72 kDa in size). Atrosimab lacks the ability to mediate Fc effector functions, due to mutations that were introduced into Fc1κ; the lack of binding to effector molecules of the immune system prevents the activation of TNFR1 due to secondary crosslinking of Atrosimab bound to cells expressing FcγRs. Atrosimab bound to TNFR1 with high affinity (K_{D} 2.7 nM), inhibited TNF-induced activation of TNFR1 with IC₅₀ values of 16-55 nM in various in vitro assays, and in the presence of anti-human IgG antibodies (i.e., cross-linking antibodies), and displayed improved pharmacokinetic properties. Compared to the parental Fab 13.7 molecule, TNFR1 binding and inhibition was slightly reduced, which can be attributed to alterations in the VH and VL pairing after fusion to the CH2 domain. The initial and terminal half-lives of Atrosimab were determined to be 2.2+/−1.2 h and 41.7+/−18.1 h, respectively, and the AUC was 5856+/−1369.9 pg/ml x h. The terminal half-life of Atrosimab was extended almost 40-fold compared to that of Fab 13.7, and was extended by 1.3-fold compared to ATROSAB; these values may be inaccurate, however, because the injected doses of Fab 13.7 and ATROSAB were lower, which can affect pharmacokinetic properties (see, e.g., Richter et al. (2019) mAbs 11(4):653-665).

ii. Domain Antibody (dAb)-Based TNFR1 antagonists

Another class of therapeutics, small fragments of antibodies, are domain antibodies (dAbs; also known as single domain antibodies, or sdAbs), which are monomeric and contain a variable domain of the heavy chain (V_{H}) or of the light chain (V_{L}) of an antibody. dAbs are the smallest antigen-binding fragments of antibodies; they are approximately 11-15 kDa in size, which is about one-tenth the size of a full monoclonal antibody (mAb). Similar to dAbs, are the nanobodies that occur in camelids, which produce antibodies that contain only heavy chains, where the antigen-binding site is a single unpaired variable domain, known as a V_{HH}. In a dAb, there are three complementarity determining regions (CDRs) on each V_{H} and each V_{L}; thus, each dAb contains three out of the six CDRs from a V_{H}-V_{L} pair in an antibody, which are the highly diversified loop regions that bind to the target antigen.

Due to their smaller size, dAbs are produced at higher yields from bacterial cultures, and are more amenable to phage display, since only a single polypeptide chain is produced. Specific dAbs with high affinities and potencies rapidly can be produced by protein engineering. The small size of dAbs also allows for increased tissue penetration, stability, and choice of delivery formulations. Due to their small size, it is possible to create molecules containing linked dAbs that are specific for different antigens/targets, which is not possible with conventional antibodies, and is difficult to achieve for other antibody fragments, such as Fabs and scFvs. Due to the monomeric and monovalent binding modality of dAbs, they are suitable for use where the targets are not amenable to intervention with monoclonal antibodies. TNFR1 is one such target; TNFR1 is activated/agonized by antibody-induced receptor cross-linking (see, e.g., Holt et al. (2003) Trends in Biotechnology 21(11):484-490; Schmidt et al. (2013) Arthritis & Rheumatism 65(9):2262-2273; Goodall et al. (2015) PLoS ONE 10(9):e0137065).

Small size antibody fragments, such as dAbs, scFvs, Fvs, disulfide-bonded Fvs and Fabs, are easier to produce and handle, and are distributed rapidly throughout the body, in comparison to larger molecules; however, their short in vivo half-life limits their therapeutic efficacy. As with other antibody fragments, increasing the serum half-life of dAbs increases the therapeutic efficacy and decreases the frequency of dosing, particularly in applications that require binding antigens in the bloodstream, such as in the treatment of rheumatoid arthritis or cancer. This can be achieved by PEGylation, conjugation to serum albumin, fusion with a second dAb with specific binding to serum albumin, or fusion to an Fc fragment or complete antibody constant regions. Fusion with an Fc region also allows for the recruitment of Fc effector functions, including complement activation, antibody-dependent cellular cytotoxicity, or Fc-mediated clearance of immune complexes (see, e.g., Holt et al. (2003) Trends in Biotechnology 21(11):484-490; Goodall et al. (2015) PLoS ONE 10(9):e0137065).

a) Anti-TNFR1 dAb-Anti-Albumin dAb Fusion Constructs

DMS5540 is a 25 kDa mouse TNFR1 antagonist, that is a bispecific single variable domain antibody, containing a noncompetitive (does not interfere with TNF binding) anti-TNFR1 dAb, fused with an albumin-binding dAb (AlbudAb; to extend serum half-life). DMS5540, which does not bind human TNFR1, was found to inhibit TNF-α-mediated cytotoxicity in the mouse fibroblast cell line L929 (which is highly sensitive to TNF-α-mediated cytotoxicity). DMS5540 was administered to mice intravenously, followed four hours later with an intravenous bolus injection of TNF-α, and serum IL-6 levels were assessed. DMS5540 demonstrated a dose-dependent inhibition of TNF-α-mediated signaling effects in vivo, as determined by a decreased IL-6 response, when compared to mice administered a control dAb lacking specific antigen binding but fused to AlbudAb (DMS5538), or no dAb (see, e.g., Goodall et al. (2015) PLoS ONE 10(9):e0137065).

In another study, mice with collagen-induced arthritis (CIA) were treated, beginning on the day of arthritis onset, for 10 days with DMS5540, an isotype (negative) control dAb (DMS5538), or murine TNFR2 genetically fused with mouse IgG1 Fc domain (mTNFRII-Fc; mTNFR2.Fc), which blocks both receptors (TNFR1 and TNFR2) and inhibits mouse TNF, and disease progression was monitored. The concentrations of systemic cytokines were measured, the numbers of T cell subsets in lymph nodes and spleens were assessed, and intrinsic Treg cell function was evaluated. Disease progression was suppressed similarly by blockade of TNFR1 with DMS5540 and blockade of TNFR1/2 with mTNFRII-Fc, compared to the negative control, indicating that blockade of TNFR1 or TNF protects joints from inflammatory mediators that result in joint damage in arthritis. Effector T cell activity, measured in terms of the expression levels of proinflammatory cytokines (e.g., IFNγ, IL-10 and RANTES), was increased following blockade of TNFR1/2 with mTNFRII-Fc, but not following the selective blockade of TNFR1 with DMS5540, indicating an immunoregulatory role (e.g., T cell effector function suppression) for TNFR2 signaling. Additionally, blockade of TNFR1, but not of TNFR1/2, resulted in the expansion and activation of Treg cells, while an increase in the expression of FoxP3 and TNFR2, both of which are expressed by Tregs, was observed in joints undergoing remission, indicating their role in the resolution of inflammation. These results indicate that inhibiting TNFR1, but not TNFR2, signaling inhibits inflammation and promotes Treg cell suppressor activity, resulting in enhanced therapeutic efficacy compared to traditional methods of TNF inhibition (see, e.g., McCann et al. (2014) Arthritis & Rheumatology 66(10):2728-2738).

DMS5540 also more effectively prevents inflammation-induced osteoclast formation and bone loss, than mTNFR2.Fc (anti-TNF), in an in vivo mouse model of lipopolysaccharide (LPS)-induced osteolysis. TNFR2-deficient mice displayed an increase in LPS-induced bone destruction. In vitro, the human equivalent of DMS5540, DMS5541, which contains an anti-human TNFR1 dAb, reduced human osteoclastogenesis in the presence and absence of low-dose TNF more effectively than etanercept. These results indicate an osteo-protective role for TNFR2 signaling. As a result, selective inhibition of TNFR1 also can be used for therapeutic intervention in inflammatory bone loss disorders, such as osteomyelitis and periprosthetic osteolysis and aseptic loosening (see, e.g., Esperito Santo et al. Biochem. Biophys. Res. Commun. 464:1145-1150).

DMS5541 (also known as TNFRI-AlbudAb), which contains a noncompetitive human TNFR1-specific dAb fused to AlbudAb, was evaluated for the selective blockade of TNF signaling via TNFR1, in ex vivo cultured human rheumatoid arthritis (RA) synovial membrane mononuclear cells (MNCs), which express TNFR1 and TNFR2, and produce inflammatory cytokines and chemokines spontaneously, in the absence of exogenous stimulation. DMS5541 inhibited the production of the proinflammatory cytokines GM-CSF, IL-10, IL-10 and IL-6, and the chemokines IL-8, RANTES (CCL5) and MCP-1 (CCL2), at similar levels to TNF ligand blockade with etanercept. This inhibition was not due to cellular toxicity, as DMS5541 inhibited TNF-α-induced cytotoxicity in human rhabdomyosarcoma KYM-1D4 cells in a dose-dependent manner, similar to TNF blockade with etanercept. In addition, DMS5541 inhibited the production of soluble TNFR1, but not soluble TNFR2, demonstrating selectivity for TNFR1. These results indicate that the TNFR1 pathway is the dominant inflammatory pathway that is responsible for the TNF response observed in the ex vivo cultured RA synovial membrane MNC disease model (see, e.g., Schmidt et al. (2013) Arthritis & Rheumatism 65(9):2262-2273).

### b) Domain Antibody Fragments Designated GSK1995057 and GSK2862277

The domain antibody fragment designated GSK1995057 (see, SEQ ID NO:55) is a short-acting, fully human domain antibody (dAb) fragment (containing a V_{H} chain) that selectively antagonizes TNF signaling through TNFR1, but not TNFR2. Due to its small size, GSK1995057 can be nebulized directly to the lungs, and has been investigated in the treatment of animal and human models of acute respiratory distress syndrome (ARDS) via inhalation. GSK1995057 reduces pulmonary inflammation in non-human primate (cynomolgus monkey) and human models of ARDS. Pulmonary neutrophil infiltration is central to the pathogenesis of ARDS, and is increased by damage to the alveolar-capillary barrier caused by the action of proinflammatory mediators. TNF-α contributes to increased endothelial permeability, and GSK1995057 prevents this increase, indicating that TNFR1 signaling mediates TNF-induced endothelial permeability (see, e.g., Proudfoot et al. (2018) Thorax 73:723-730). Because of its inherent short half-life and neutralization by auto-antibody, the trial failed. The immunogenicity of GSK1995057 may be due more to improper folding of the protein made in E. coli than as a failure to properly humanize the dAb; it was derived from a human antibody fragment, and only the hypervariable sequences were altered to adapt specificity for TNFR1 (see, e.g., International PCT Publication No. WO2008/149148A2).

In monkeys exposed to a single inhaled lipopolysaccharide (LPS) challenge, which is a well-established model that triggers a clinically relevant inflammatory response modeling subclinical tissue injury, pretreatment with GSK1995057 reduces pulmonary neutrophil infiltration, levels of proinflammatory chemokines, markers of endothelial injury and alveolar-capillary leak, in a dose-dependent manner. The results indicate that inhaled GSK1995057 can effect the same results as higher doses of parenterally administered antibodies. In a clinical trial, in which healthy human subjects were pretreated with a single nebulized dose of GSK1995057 and then exposed to a low dose of inhaled LPS, the pretreated subjects experienced less systemic inflammation, as well as less neutrophilic lung inflammation and signs of endothelial injury in response to LPS challenge, in comparison to subjects who received a placebo. Despite these results, translation into the clinic is not likely. In the trial GSK1995057 was administered prior to the LPS challenge, but patients with ARDS, generally require treatment after the initial injury (see, e.g., Proudfoot et al. (2018) Thorax 73:723-730), not before.

Another difficulty is the detrimental effects of anti-drug antibodies (ADAs) on anti-TNFR1 agents, which were observed in a clinical Phase I study of GSK1995057, in which cytokine release infusion reactions, at doses of 2-10 pg/kg, were observed due to high levels of pre-existing, naturally occurring anti-immunoglobulin autoantibodies, (i.e., ADAs) present in approximately 50% of drug naive, healthy subjects. Specifically, the ADAs were human anti-V_{H} (HAVH) autoantibodies, and the complex of HAVH autoantibodies with framework sequences of GSK1995057 resulted in the activation of TNFR1 signaling, and the occurrence of mild to moderate infusion reactions in subjects with high HAVH autoantibody titers (see, e.g., Cordy et al. (2015) Clin. Exp. Immunol. 182:139-148).

The binding of HAVH autoantibodies to a framework region of the dAb GSK1995057 induces cytokine release in vitro. The epitope on GSK1995057 for the autoantibodies was characterized. Pre-existing anti-drug antibodies (ADAs) bind to an epitope close to the C-terminal regional of V_{H} dAbs, including the dAb GSK1995057. To counter this, a modified dAb, designated GSK2862277 (see, SEQ ID NO:56) was generated by adding a single alanine residue at the C-terminus of the modified dAb. This modification reduced binding to HAVH autoantibodies. In serum samples from healthy subjects that screened positive for HAVH autoantibodies that bind to GSK1995057, the frequency of pre-existing autoantibodies decreased from 51% for GSK1995057-specific HAVH autoantibodies, to 7% for GSK2862277-specific autoantibodies. Human in vitro systems and animal in vivo experiments showed that GSK2862277 does not induce TNFR1 activation, even in the presence of GSK2862277-specific autoantibodies, and that the pharmacology and biophysical properties of GSK2862277, including target affinity, in vitro potency and in vivo pharmacokinetics and pharmacodynamics, is comparable to those of the parent dAb (GSK1995057).

A Phase I clinical trial to investigate the safety, tolerability, pharmacokinetics and pharmacodynamics of single and repeat doses of inhaled (i.h.) and intravenous (i.v.) GSK2862277 found that GSK2862277 was generally well tolerated when administered via inhalation or intravenously. One subject, however, had a mild infusion reaction with cytokine release after repeated IV dosing; this subject had high serum levels of pre-existing antibodies to GSK2862277, and the serum antibodies from this subject were shown to activate TNFR1 signaling in an in vitro assay. The interaction between GSK2862277 and the autoantibodies result in antibody-mediated, GSK2862277-dependent cross-linking of cellular TNFR1, agonizing the receptor and leading to cytokine release. Thus, despite the reduced binding of GSK2862277 to pre-existing HAVH autoantibodies, adverse effects were still associated with the presence of a new pre-existing antibody response, that was specific to the modified dAb framework. These results highlight challenges in developing biological antagonists against TNFR1 (see, e.g., Cordy et al. (2015) Clin. Exp. Immunol. 182:139-148). Thus, there remains a need for improved TNFR1 antagonists.

iii. Nanobodies (Nbs)

Similar to dAbs, nanobodies (Nbs) are small antigen-binding fragments derived from camelid heavy-chain antibodies that are devoid of light chains. They are small (15 kDa), have low immunogenicity and high affinity, are soluble and stable, and are encoded by a single gene/exon (VHH), making them modular and allowing for high yield production in bacteria or yeasts.

iv. Anti-TNFR1 Nanobody-Anti-Albumin Nanobody Fusion Constructs

TROS (TNF Receptor One-Silencer; also called Nb Alb-70-96) is a trivalent high-affinity nanobody-based selective inhibitor of human TNFR1 that competes with TNF for binding to TNFR1. To generate TROS, two anti-human TNFR1 nanobodies (Nb 70 and Nb 96; see, SEQ ID NOs: 683 and 684, respectively), which had been generated from a VHH library that was constructed by immunization of alpacas with recombinant human soluble TNFR1, were linked and, via (Gly₄Ser)₃ linkers, were linked to an anti-albumin nanobody (Nb Alb) to increase serum half-life to produce the trivalent TROS. The serum half-life of the resulting TROS is ~24 hours; the serum half-life of monovalent Nbs is only ~1.5 hours. Treatment with TROS delays disease onset in mouse experimental autoimmune encephalomyelitis (EAE; a model of MS), and prevents established disease; the therapeutic effects are due to the diversion of TNF to signal through TNFR2, and the effects of such signaling. TROS also inhibits inflammation in ex vivo cultured colon biopsies of Crohn's disease patients, and antagonizes inflammation in a model of acute TNF-induced liver inflammation in liver chimeric humanized mice (see, e.g., Steeland et al. (2015) J. Biol. Chem. 290(7):4022-4037; Steeland et al. (2017) Sci. Reports 7:13646).

c. Dominant-Negative Inhibitors of TNF (DN-TNFs)/TNF Muteins

Another class of TNF inhibitors are the signaling-incompetent dominant-negative inhibitors of TNF (DN-TNFs), also known as TNF muteins. The DN-TNFs are engineered variants of TNF with mutations that abrogate binding to and signaling through TNFR1 and TNFR2. DN-TNFs selectively inhibit soluble TNF (sTNF or solTNF) by rapidly exchanging subunits with native TNF homotrimers, forming inactive mixed TNF heterotrimers with disrupted receptor binding surfaces, thus preventing interaction with TNF receptors. DN-TNFs leave transmembrane TNF (tmTNF) unaffected, maintaining the protective roles of TNF signaling through TNFR2. DN-TNFs inhibit TNF-induced NF-cB activity and caspase-mediated apoptosis, and reduce disease severity in animal models of arthritis and Parkinson's disease. These molecules because of their structure likely are immunogenic.

As selective inhibitors of soluble TNF, DN-TNFs, unlike anti-TNF therapies that bind to solTNF and to tmTNF, do not inhibit tmTNF signaling, and do not suppress the resistance of mice to infection by L. monocytogenes. Examples of DN-TNFs are TNF mutants containing one or more of the replacements L133Y, S162Q, Y163H, I173T, Y191Q and A221R, with reference to the sequence of amino acids set forth in SEQ ID NO:1 (corresponding to residues L57Y, S86Q, Y87H, I97T, Y115Q, and A145R, with reference to the sequence of solTNF, as set forth in SEQ ID NO:2), which impair binding to TNFRs. Additional modifications, for example, to improve expression, allow site-specific PEGylation, also can be included (see, e.g., Zalevsky et al. (2007) J. Immunol. 179:1872-1883).

For example, the TNF mutations R32W and S86T, with reference to SEQ ID NO:2, result in a several hundred-fold loss in affinity towards TNFR2, but do not affect binding to TNFR1. The R32W/S86T double mutant abrogates all binding to TNFR2, with no loss in binding to TNFR1. The mutations L29S, L29G, L29Y, R31E, R31N, R32Y, R32W, S86T, L29S/R32W, L29S/S86T, R32W/S86T, L29S/R32W/S86T, R31N/R32T, R31E/S86T, R31N/R32T/S86T, and E146R, with reference to SEQ ID NO:2, also impart selectivity towards TNFR1. The mutations D143N, D143Y, A145R and D143N/A145R, with reference to SEQ ID NO:2, render the TNF variants selective for TNFR2 (see, e.g., Loetscher et al. (1993) J. Biol. Chem. 268(35):26350-26357; U.S. Pat. No. 5,422,104).

A modified TNF, designated XPro1595 (INmuneBio; see, SEQ ID NO:701), is a PEGylated, soluble DN-TNF mutein that preferentially inhibits TNFR1 signaling, and contains the mutations ViM, R31C, C69V, Y87H, C101A and A1456R, with reference to SEQ ID NO:2 (see, e.g., U.S. Publication No. 2015/0239951). XPro1595 decreases neuroinflammation and is being investigated in the treatment of Alzheimer's disease (see, e.g., clinical trial identifier No. NCT03943264). XPro1595 blocks the development of amyloid pathology in a mouse model of Alzheimer's Disease (3×TgAD), prevents the loss of neuron communication and cognitive impairment in a different (tgCRND8) mouse model of Alzheimer's Disease, attenuates the dysfunction in neuronal communication and cognitive deficit in normal aged rats, and prevents young mice from developing amyloid pathology, cognitive impairment, and dysfunction in neuronal communication, in a third model (5×FAD) of Alzheimer's disease. In older mice that have Alzheimer's-like pathology, XPro1595 reduced amyloid, improved cognition, rescued neuron communication, and also, normalized innate and adaptive immune responses.

The levels of TNFR1 are higher in the hippocampus, in comparison to TNFR2, in aged (22 months) but not young adult (6 months) Fischer 344 rats. When treated with XPro1595, aged rats exhibit improved Morris Water Maze performance, reduced microglial activation, reduced susceptibility to hippocampal long-term depression, increased levels of the GluR1 type glutamate receptors, and lower L-type voltage sensitive Ca²⁺ channel (L-VSCC) activity in hippocampal CA1 neurons, indicating that functional changes associated with brain aging can occur from selective alterations in TNF signaling. In animal models of Parkinson's disease and aging, XPro1595 suppresses neuroinflammation and the activation of microglia. In EAE (a model of MS), XPro1595 ameliorates disease, improves remyelination and reduces CNS lesions and neuroinflammation. XPro1595 also ameliorates inflammatory arthritis, and decreases susceptibility to infection in treated animals. In comparison to etanercept, which had no therapeutic effect, treatment with XPro1595 delayed the onset of EAE and ameliorated symptoms more efficiently. XPro1595 administration increases the level of TNFR2 expression in the lesion area in EAE, indicating that tmTNF signaling via TNFR2 is implicated in neural regeneration (see, e.g., Yang et al. (2018) Front. Immunol. 9:784; Sama et al. (2012) PLoS ONE 7(5):e38170). Since XPro1595 does not inhibit the activity of transmembrane TNF (which activates TNFR1 and TNFR2), it cannot block the inflammatory effects of TNFR1. This also applies to other dominant negative TNF reagents, described below.

XENP345 (see, SEQ ID NO:702) is a PEGylated DN-TNF mutein, containing the mutations I97T/A145R, with reference to SEQ ID NO:2. The in vivo neutralization of soluble TNF (solTNF) by XENP345 in animal models of Parkinson's disease and Alzheimer's disease is neuroprotective, reduces neuronal degeneration and cognitive dysfunction, and slows down neurodegenerative disease progression (see, e.g., McCoy et al. (2006) J. Neurosci. 26(37):9365-9375; McAlpine et al. (2009) Neurobiol. Dis. 34(1):163-177).

R1antTNF (see, SEQ ID NO: 703) is a TNFR1-selective antagonistic mutant TNF, identified from a phage library displaying structural human TNF variants in which each of the six amino acid residues at the receptor-binding site, corresponding to residues 84-89 of SEQ ID NO:2, were mutated. R1antTNF, which contains the mutations A84S, V85T, S86T, Y87H, Q88N and T89Q, has similar affinity to TNFR1 as wild-type human TNF, and does not interfere with TNFR2 activity. R1antTNF ameliorated liver injury, as evidenced by reductions in the serum levels of alanine aminotransferase and the pro-inflammatory cytokines IL-2 and IL-6, in two models of acute hepatitis. The plasma half-life of R1antTNF, like wild-type TNF, however, is very short (12 min). To increase the in vivo half-life of R1antTNF, a PEGylated version, PEG-R1antTNF, in which PEG is bound to the N-terminal site of R1antTNF, was produced. PEG-R1antTNF decreases morbidity, ameliorates disease symptoms, improves demyelination in an EAE mouse model, and suppresses Th1 and Th17 cell activation and inflammatory T-cell infiltration in the spinal cord. PEG-R1antTNF also inhibits NF-κB, suppresses smooth muscle cell proliferation, and decreases chemokine and adhesion molecule expression, thus decreasing intimal hyperplasia and arterial inflammation in IL-1 receptor antagonist-deficient mice after inducing femoral artery injury in an external vascular cuff model. When the effects on antiviral immunity of PEG-R1antTNF and etanercept were compared using a recombinant adenovirus vector, PEG-R1antTMF did not reactivate viral infection and did not affect the clearance of injected adenovirus, while viral load increased after treatment with etanercept. PEG-R1antTNF treatment also delayed and ameliorated CIA symptoms in prophylactic and therapeutic settings, and was more effective than etanercept when used for the treatment of established CIA (see, e.g., Yang et al. (2018) Front. Immunol. 9:784; Shibata et al. (2008) J. Biol. Chem. 283(2):998-1007; Kitagaki et al. (2012) J Atheroscler. Thromb. 19(1):36-46; Fischer et al. (2015) Antibodies 4:48-70; Horiuchi et al. (2010) Rheumatology (Oxford) 49:1215-1228).

Soluble TNFR1 also has been associated with an increased risk of developing MS; thus, neutralization of soluble TNFR1, which cannot be achieved with DN-TNFs/TNF muteins, can be beneficial. In contrast to inhibitors of solTNF, such as DN-TNFs, TNFR1 antagonists can block the binding of lymphotoxin-a (LT-a), another member of the TNF superfamily, to TNFR1. LT-a can have a proinflammatory role in RA and in animal disease models, such as CIA and EAE; thus, simultaneous blocking of TNF and LT-a binding to TNFR1 by TNFR1 antagonists can have additional benefits, in comparison to solTNF inhibition, in acute and chronic inflammatory diseases and disorders (see, e.g., Fischer et al. (2015) Antibodies 4:48-70).

### 2. TNFR2-Selective Agonists

CD4⁺FoxP3⁺regulatory T cells (Tregs) maintain immunological homeostasis and inhibit autoimmune responses; Tregs also modulate the antitumor immune response, allowing for tumor immune evasion. Tregs, thus, are a therapeutic target in the treatment of, for example, autoimmune and chronic inflammatory diseases and conditions, graft-versus-host disease (GvHD), transplantation rejection, and cancer. TNF signaling via TNFR2 regulates the function and activity of Tregs. TNFR2 agonists upregulate Treg activity, while TNFR2 antagonists downregulate Treg activity. The Treg-stimulatory effect of the TNF-TNFR2 signaling pathway can be leveraged for the treatment of several human diseases and disorders, including autoimmune and chronic inflammatory diseases, through agonism, and cancer, through antagonism (see, e.g., Zou et al. (2018) Front. Immunol. 9:594).

TNFR2 agonists include antibodies, such as monoclonal TNFR2 agonist antibodies, and antigen-binding fragments thereof, peptides and proteins, such as TNFR2-selective TNF muteins, fusion proteins, and small molecules. As provided herein, specific agonism of TNFR2 induces the expansion and activation of Tregs, which modulate the immune system, reduces the activity of autoreactive CD8⁺ T cells that damage tissues, and induces signaling pathways with anti-inflammatory, as well as cell survival, regeneration and protective effects, including neuro-protective, cardio-protective, gut-protective and osteo-protective effects. Thus, the enhancement of TNFR2 signaling with TNFR2-selective agonists can be used to enhance the therapeutic effects of TNFR1-specific antagonism, particularly in the treatment of autoimmune and chronic inflammatory diseases and disorders, including neurodegenerative diseases in which anti-TNF therapies/TNF-blockers have failed.

a. TNFR2 agonistic Antibodies

Human TNFR2-selective agonist antibodies include the commercially available MR2-1 (a monoclonal mouse IgG1 that binds human, cynomolgus monkey and rhesus monkey TNFR2; Hycult Biotech), and clone MAB2261 (a monoclonal mouse IgG2A that binds human TNFR2; R&D Systems). TNFR2 agonists, such as antibodies, can potently stimulate the expansion of homogeneous populations of FoxP3⁺Tregs in CD4 cell cultures, and upregulate the expression of TNF, TRAF2, TRAF3, BIRC3 (cIAP2) and FoxP3 mRNA. Magnetic-activated cell sorting (MACS)-purified CD4⁺CD25⁺cells, cultured using standard in vitro human Treg expansion protocols (i.e., with anti-CD3 antibodies, anti-CD28 antibodies, IL-2 and rapamycin), yield expanded Tregs with higher levels of FoxP3 (and other characteristic Treg markers), and more potent suppressive capacities, when expanded in the presence of a TNFR2 agonist antibody, compared to in the absence of the TNFR2 agonist. Tregs isolated from a patient with type 1 diabetes, that exhibit a resting phenotype, are activated and expanded upon in vitro treatment with a TNFR2 agonist antibody; such Tregs are more potent in the inhibition of autologous CD8⁺ T cells (see, e.g., Zou et al. (2018) Front. Immunol. 9:594).

Treatment of isolated Tregs, expanded using the standard in vitro protocol, with MR2-1, a commercially available agonistic human TNFR2 monoclonal antibody (mAb) containing a mouse IgG1, generates homogenous populations of FoxP3⁺Helios⁺CD127^{low} Tregs; these Tregs maintain their phenotype and highly suppressive activity in a humanized mouse model. TNFR2 agonists, thus, can enhance the ex vivo expansion of Treg cells from impure cell populations, for use in Treg-based immunotherapy (see, e.g., Zou et al. (2018) Front. Immunol. 9:594).

b. TNFR2-Selective TNF Muteins and Fusions Thereof

As described herein, TNF can be engineered to selectively bind TNFR1 or TNFR2; for example, a TNFR2 selective TNF mutein is a variant of TNF that contains one or more mutations that increase binding to TNFR2 and/or reduce or eliminate binding to TNFR1. TNFR2-selective mutations include non-conservative substitutions of the Asp residue at position 143 of soluble TNF (see, SEQ ID NO:2), such as, for example, D143Y, D143F or D143N, or non-conservative substitutions of the Ala residue at position 145 of soluble TNF, such as, for example, A145R (see, e.g., U.S. Pat. No. 9,081,017). Other mutations in TNF that impart selectivity for TNFR2 include, but are not limited to, for example, K65W, D143E, D143W, D143V, A145H, A145K, A145F, A145W, E146Q, E146H, E146K, E146N, D143N/A145R, A145R/S147T, Q88N/T89S/A145S/E146A/S147D, Q88N/A1451/E146G/S147D, A145H/E146S/S147D, A145H/S147D, L29V/A145D/E146D/S147D, A145N/E146D/S147D, A145T/E146S/S147D, A145Q/E146D/S147D, A145T/E146D/S147D, A145D/E146G/S147D, A145D/S147D, A145K/E146D/S147T, A145R/E146T/S147D, A145R/S147T, E146D/S147D, D143V/F144L/A145S, and D143V/A145S, with reference to SEQ ID NO:2 (see, e.g., U.S. Patent Publication No. 2020/0102362).

TNF ligand trimerization is essential for signaling via TNFRs. At low concentrations, such as in serum, the trimers dissociate, resulting in their degradation.

To generate functionally active, receptor-specific TNF muteins, it is necessary to create stable trimers. TNF07 is a soluble TNF (sTNF or solTNF) mutein, containing the mutations S95C/G148C (with respect to the sequence of residues set forth in SEQ ID NO:2), that forms a stable TNF trimer and functions as a TNFR2 agonist. The S95C/G148C mutations result in the formation of an intermolecular Cys-Cys covalent bond; a stable trimer is thus formed as a result of covalent internal disulfide cross-linking of sTNF at a strategic location between TNF monomers. TNF07 acts as a TNFR2 agonist despite lacking TNFR2-selective mutations. TNF07 induces potent TNFR2 signaling, expands FoxP3⁺Treg cells, and selectively induces the death of autoreactive CD8⁺ T cells isolated from patients with type 1 diabetes (see, e.g., Ban et al. (2015) Molecular and Cellular Therapies 3:7; Zou et al. (2018) Front. Immunol. 9:594).

Several TNFR2 agonists, containing fusions of single-chain TNFR2-selective TNF mutein trimers, with multimerization domains, have been generated. As described herein, the primary ligand for TNFR2 is membrane-bound TNF (memTNF; also referred to herein as transmembrane TNF or tmTNF). The addition of multimerization domains, such as dimerization or trimerization domains, generates hexameric or nonameric molecules, respectively, with respect to the TNF subunits; these hexamers and nonamers of TNF mimic membrane-bound TNF trimers and thus, are capable of effectively activating TNFR2 signaling. Commonly used dimerization domains include EHD2, which is derived from the heavy chain CH2 domain of IgE and MHD2, which is derived from the heavy chain CH2 domain of IgM. Dimerization domains also can include Fc domains, such as those derived from IgG1 and IgG4, optionally including modifications that alter immune effector functions. Commonly used trimerization domains include chicken tenascin C (TNC) and human TNC.

Dimerization and trimerization enhances TNFR2 signaling, and improves the half-life of the fusion protein, for example, by increasing the molecular weight of the molecule, and/or by introducing FcRn recycling, for example, when the dimerization domain is an Fc.

STAR2 (also known as TNC-sc-mTNF(221N/223R)) is a nonameric agonistic TNFR2-specific mouse TNF variant that does not bind TNFR1, and is a single-chain mouse TNF timer, where each TNF subunit is residues 91-235 of SEQ ID NO:5, fused to the trimerization domain of chicken tenascin C (cTNC), corresponding to residues 110-139 of SEQ ID NO:804 (see, also, SEQ ID NO:805). The three single-chain mouse TNF subunits are linked by two (GGGS)₄ peptide linkers (see, e.g., SEQ ID NO:707. Residues 116-120), and the TNC trimerization domain is linked to the N-terminus of the first TNF subunit in the single-chain trimer. The specificity of STAR2 for TNFR2 results from the mutations D221N and A223R (with reference to the sequence of mouse TNF, set forth in SEQ ID NO:5) within the individual TNF subunits, which creates a steric clash between STAR2 and mouse TNFR1. Fusion to the TNC trimerization domain causes spontaneous oligomer formation, creating three covalently linked TNF trimers, and mimicking membrane-bound TNF. STAR2 stimulates the proliferation of Tregs in vitro and in vivo in a TNFR2-dependent, IL-2-independent mechanism. Pretreatment of allogeneic hematopoietic stem cells with STAR2 prior to transplantation in mice prolonged the survival and decreased the severity of GvHD in a TNFR-2 and Treg-dependent manner. A human equivalent of the TNFR2-specific STAR2 agonist, TNC-scTNF(143N/145R), made of residues 9-157 of soluble TNF (see, SEQ ID NO:2), containing the mutations D143N/A145R with reference to SEQ ID NO:2 (solTNF), potently stimulated CD4⁺FoxP3⁺Treg expansion in vitro from CD4⁺ T cells isolated from healthy donors (see, e.g., Chopra et al. (2016) J. Exp. Med. 213(9):1881-1900; Zou et al. (2018) Front. Immunol. 9:594).

TNC-scTNF_{R2} is a soluble human TNFR2 agonist that is a fusion of the trimerization domain of human tenascin C (hTNC), containing residues 110-139 of SEQ ID NO:806 (see, also, SEQ ID NO:807), to the N-terminus of a TNFR2-selective single-chain TNF variant (scTNF_{R2}; SEQ ID NO:803), contains three TNF domains connected by two short peptide linkers (GGGGS). The TNFR2-selective TNF molecule, scTNF_{R2}, resembles soluble trimeric TNF, and each TNF subunit includes amino acids 80-233 of the full length TNF set forth in SEQ ID NO:1 (corresponding to residues 4-157 of SEQ ID NO:2), with the mutations D143N/A145R, with reference to SEQ ID NO:2, which eliminate binding to TNFR1. Because TNFR2 is only fully activated by membrane-bound TNF, but not soluble TNF trimers, the trimerization domain of TNC is fused to the N-terminus of scTNF_{R2}, generating TNC-scTNF_{R2}. TNC-scTNF_{R2} exists in a trimeric assembly of the single stranded fusion protein and resembles a nonameric TNF molecule; this oligomeric TNF mutein, due to its increased avidity, mimics membrane-bound TNF (memTNF) activity, induces the clustering of TNFR2 and the formation of TNFR2 signaling complexes, efficiently activating TNFR2. TNC-scTNF_{R2} exhibits neuroprotective properties; it preserves neurons from superoxide-induced cell death and rescues neurons from catecholaminergic cell death. In an in vitro model of Parkinson's disease, TNC-scTNFR2 rescued neurons after induction of cell death by 6-OHDA. These results indicate that TNC-scTNF_{R2} can ameliorate neurodegenerative processes (see, e.g., Fischer et al. (2011) PLoS ONE 6(11):e27621).

EHD2-scTNF_{R2} (see, SEQ ID NO:810) is an agonistic TNFR2-selective TNF mutein fusion protein that contains a covalently stabilized human TNFR2-selective single-chain TNF trimer (scTNF_{R2}; SEQ ID NO:803) with the mutations D143N/A145R (residue numbering with respect to soluble TNF, as set forth in SEQ ID NO:2), which abrogate binding to TNFR1, fused to the dimerization domain EHD2 (SEQ ID NO:808), which is derived from the heavy chain C_{H}2 domain of IgE, and creates a disulfide bonded dimer that contains hexameric TNF domains. Each TNF subunit within scTNF_{R2} contains residues 4-157 of SEQ ID NO:2. EHD2 is fused to the N-terminal end of the trivalent human single-chain scTNF_{R2} via a peptide linker (GGGSGGGSGGGSGGGSGGGSGGSEFLA; SEQ ID NO:809), and the three TNF domains of scTNF_{R2} are connected via two GGGGS peptide linkers. EHD2-scTNFR2 exhibits neuroprotective properties in a mouse model of NMDA-induced acute neurodegeneration (see, e.g., Dong et al. (2016) Proc. Natl. Acad. Sci. U.S.A. 113(43):12304-12309; and U.S. Patent Publication No. 2020/0102362).

TNFR2 agonist fusion proteins also include single chain TNFR2 agonists (scTNF_{R2}) containing three TNF muteins with the mutations D143N/A145R (with reference to SEQ ID NO:2), which abrogate binding to TNFR1, fused with a dimerization domain that is an Fc, resulting in a protein that is hexameric with respect to the TNF domains (scTNF_{R2}-Fc). The Fc can be an IgG4 or IgG1 Fc, optionally containing mutations that eliminate Fc effector functions, such as ADCC and CDC. The three TNF muteins, which contain residues 12-157 of SEQ ID NO:2, are linked together by two short peptide linkers, and the dimerization domain is linked to the N-terminus or C-terminus of the single chain trimeric TNF molecule (scTNF_{R2}) by a third short peptide linker. The three linkers can all be the same or can be different, and can include GS linkers, such as (GGGGS)~residues 116-121 of SEQ ID NO:707, and/or other combinations of Gly and Ser, where n=1-5, or can include all or a portion, at least 10, 15, or 20 contiguous residues, of the stalk region of TNF-α (GPQREEFPRDLSLISPLAQAVRSSSRTPSDK (SEQ ID NO:812), corresponding to residues 57-87 of SEQ ID NO:1). Dimerization enhances signaling by the TNFR2 agonist, and also improves the half-life of the fusion protein. Alternative dimerization domains that can be used in the fusion proteins include Fc fusion proteins derived from other dimerizing molecules, such as the IgE heavy chain domain 2 (EHD2; see, SEQ ID NO:808) and IgM heavy chain domain 2 (MHD2; see, SEQ ID NO:811) (see, e.g., International Application Publication No. WO 2019/226750).

### 3. Anti-TNFR2 Antagonistic Antibodies and Small Molecule Inhibitors

TNFR2 antagonists inhibit the proliferation of and induce the death of Tregs, and also can inhibit the proliferation of and induce the death of TNFR2-expressing tumor cells. TNFR2 antagonists can reduce or inhibit the proliferation of myeloid-derived suppressor cells (MDSCs), and/or induce apoptosis within MDSCs, by binding TNFR2 expressed on the surface of MDSCs present in the tumor microenvironment. TNFR2 antagonists also induce the expansion of T effector cells, including cytotoxic CD8⁺ T cells, via the inhibition of Treg expansion and activity. As a result, TNFR2 antagonists can be useful in the treatment of infectious diseases, and certain cancers that express TNFR2, such as, for example, T cell lymphomas (e.g., Hodgkin's lymphoma and cutaneous non-Hodgkin's lymphoma), ovarian cancer, colon cancer, multiple myeloma, renal cell carcinoma, breast cancer, cervical cancer, endometrial cancer, glioma, head and neck cancer, liver cancer, and lung cancer (see, e.g., U.S. Patent Publication No. 2019/0144556; Torrey et al. (2017) Sci. Signal. 10:eaaf8608).

As discussed herein, expression of TNFR2 is restricted to particular immune cells, including Tregs and MDSCs, endothelial cells, and particular neurons and cardiac cells. The restricted expression of TNFR2 makes it an ideal drug target, as systemic toxicity from anti-TNFR2 therapeutics is less likely to occur.

TNFR2 antagonist antibodies and antigen-binding fragments thereof bind epitopes within human TNFR2 that contain one or more of the residues KCRPG (corresponding to residues 142-146 of SEQ ID NO:4), or a larger epitope, containing residues 130-149, 137-144 or 142-149, or at least 5 continuous or discontinuous residues within these epitopes, for example, and do not bind to the epitope containing residues KCSPG (corresponding to residues 56-60 of SEQ ID NO:4). TNFR2 antagonists also can bind the TNFR2 epitopes PECLSCGS (corresponding to residues 91-98 of SEQ ID NO:4), RICTCRPG (corresponding to residues 116-123 of SEQ ID NO:4), CAPLRKCR (corresponding to residues 137-144 of SEQ ID NO:4), LRKCRPGFGVA (corresponding to residues 140-150 of SEQ ID NO:4), and VVCKPCAPGTFSN (corresponding to residues 159-171 of SEQ ID NO:4), and/or an epitope containing at least 5 continuous or discontinuous residues within residues 75-128, 86-103, 111-128, or 150-190 of SEQ ID NO:4 (see, e.g., U.S. Patent Publication No. 2019/0144556).

In general, antagonistic TNFR2 antibodies or antigen-binding fragments thereof bind to an epitope containing one or more residues of the KCRPG sequence (SEQ ID NO:840), with an affinity that is at least 10-fold greater, for example, than the affinity of the same antibody or antigen-binding fragment for a peptide that contains the KCSPG sequence of human TNFR2 (SEQ ID NO:839). Antibodies or antibody fragments that bind epitopes containing one or more residues of the KCRPG sequence, and epitopes containing the KCSPG motif with similar affinity (e.g., less than a 10-fold difference in affinity), are not antagonistic TNFR2 antibodies. Antagonistic TNFR2 antibodies include TNFRAB1 (see, SEQ ID NOs:1213 and 1213 for the sequences of the heavy and light chains of TNFRAB1, respectively), TNFRAB2 and TNFR2A3 (see, e.g., U.S. Patent Publication No. 2019/0144556 for descriptions of these antibodies). TNFR2 antagonists also include antibodies and antibody fragments that contain the CDR-H3 sequence of TNFRAB1 (QRVDGYSSYWYFDV; corresponding to residues 99-112 of SEQ ID NO:1212), TNFRAB2 (ARDDGSYSPFDYWG; SEQ ID NO:1217) or TNFR2A3 (ARDDGSYSPFDYFG; SEQ ID NO:1223), or a CDR-H3 sequence with at least about 85% sequence identity thereto. TNFRAB1, for example, specifically binds residues 130-149, containing residues KCRPG of TNFR2, with a 40-fold higher affinity than residues 48-67, containing residues KCSPG of TNFR2 (see, e.g., U.S. Patent Publication No. 2019/0144556).

TNFRAB1 (see, SEQ ID NOs: 1212 and 1213 for heavy and light chains, respectively) is a murine antibody that antagonizes the TNF-TNFR2 interaction, and, in addition to binding the KCRPG sequence of TNFR2, also binds an epitope within residues 161-169 (CKPCAPGTF; SEQ ID NO:1258) of TNFR2 (SEQ ID NO:4). TNFRAB2, another antagonistic TNFR2 antibody, binds the epitope containing residues 137-144 (CAPLRKCR; SEQ ID NO:851), as well as epitopes that include one or more residues within positions 80-86 (DSTYTQL; SEQ ID NO:1247), 91-98 (PECLSCGS; SEQ ID NO:1248), and 116-123 (RICTCRPG; SEQ ID NO: 1249) of human TNFR2. TNFR2A3 is a murine antagonistic human TNFR2 antibody that was discovered by immunization of a mouse with human TNFR2 and subsequent CDR mutagenesis, in which the CDR-H3 of the generated precursor antibody was replaced with the CDR-H3 sequence ARDDGSYSPFDYFG (SEQ ID NO:1223). TNFR2A3 binds to two distinct epitopes within human TNFR2; the first epitope includes residues 140-150 of human TNFR2 (LRKCRPGFGVA; SEQ ID NO:1463) and contains the KCRPG motif, and the second epitope is a downstream sequence that contains residues 159-171 of human TNFR2 (VVCKPCAPGTFSN; SEQ ID NO:1464). These data indicate that the CDR-H3 sequence of an antagonistic TNFR2 antibody largely dictates the antigen-binding properties, and that the CDR-H3 motif is a modular domain that can be substituted into anti-TNFR2 antibodies that do not exhibit antagonistic activity, in order to impart such antibodies or antigen-binding fragments thereof with TNFR2 dominant antagonistic features. For example, replacement of the CDR-H3 sequence of a neutral anti-TNFR2 antibody (i.e., an antibody that is neither antagonistic nor agonistic), with the CDR-H3 of an antagonistic TNFR2 antibody, such as the CDR-H3 sequences of TNFRAB1, TNFRAB2 or TNFR2A3, for example, converts the phenotype-neutral antibody to an antagonistic TNFR2 antibody, such as a dominant antagonistic TNFR2 antibody, which is an antagonist that inhibits TNFR2 activation even in the presence of a TNFR2 agonist, such as TNF, or IL-2 (see, e.g., U.S. Patent Publication No. 2019/0144556).

TNFR2 antagonist antibodies or antigen-binding fragments thereof can contain the CDR-H1 sequences set forth in any of SEQ ID NOs: 1214, 1215, and 1231-1233; the CDR-H2 sequences set forth in any of SEQ ID NOs: 1216, 1224, and 1230; the CDR-H3 sequences set forth in any of SEQ ID NOs: 1217, 1223, and 1225-1229, or the CDR-H3 of TNFRAB1, corresponding to residues 99-112 of SEQ ID NO:1212; the CDR-L1 sequences set forth in any of SEQ ID NOs: 1218 and 1234-1236, or the CDR-L1 sequence of TNFRAB1, corresponding to residues 24-33 of SEQ ID NO:1213; the CDR-L2 sequences set forth in any of SEQ ID NOs: 1219, 1220, 1237 and 1238, or the CDR-L2 sequence of TNFRAB1, corresponding to residues 49-55 of SEQ ID NO:1213; or the CDR-L3 sequences set forth in any of SEQ ID NOs: 1221, 1222, and 1241-1244, or the CDR-L3 sequence of TNFRAB1, corresponding to residues 88-96 of SEQ ID NO:1213. Exemplary framework regions that can be used for the development of a humanized anti-TNFR2 antibody, containing one or more of the above CDRs include, without limitation, those described in U.S. Pat. Nos. 7,732,578 and 8,093,068, and in International Application Publication No. WO 2003/105782. Another approach to engineering humanized anti-TNFR2 antagonistic antibodies is to align the sequences of the heavy chain variable region and light chain variable region of an antagonistic TNFR2 antibody, such as TNFRAB1, TNFRAB2, or TNFR2A3, with the heavy chain variable region and light chain variable region of a consensus human antibody. Consensus human antibody heavy chain and light chain sequences are known in the art (see e.g., the “VBASE” human germline sequence database; see also Kabat, et al., Sequences of Proteins of Immunological Interest, Fifth Edition, U.S. Department of Health and Human Services, NIH Publication No. 91-3242, (1991); Tomlinson et al., (1992) J. Mol. Biol. 227:776-798; and Cox et al., (1994) Eur. J. Immunol. 24:827-836). In this way, the variable domain framework residues and CDRs can be identified by sequence alignment. One can substitute, for example, the CDR-H3 of the consensus human antibody with the CDR-H3 of an antagonistic TNFR2 antibody, such as the CDR-H3 of TNFRAB1, TNFRAB2, or TNFR2A3, to produce a humanized TNFR2 antagonist antibody. Exemplary variable domains of a consensus human antibody include the heavy chain variable domain set forth in SEQ ID NO:1245, and the light chain variable domain set forth in SEQ ID NO:1246, identified in U.S. Pat. No. 6,054,297 (see, e.g., U.S. Patent Publication No. 2019/0144556). The CDR-H1 and CDR-H2 sequences of the exemplary consensus sequence of a human antibody heavy chain variable domain of SEQ ID NO:1245 can be replaced, for example, with the corresponding CDR sequences of a phenotype-neutral, TNFR2-specific antibody, and the CDR-L1, CDR-L2 and CDR-L3 sequences of the exemplary consensus sequence of a human antibody light chain variable domain of SEQ ID NO:1246 can be replaced with the corresponding CDR sequences of a phenotype-neutral, TNFR2-specific antibody, to produce humanized, antagonistic TNFR2 antibodies.

Other TNFR2 antagonists can be identified by screening for peptides that bind epitopes within TNFR2, such as those set forth in any one of SEQ ID NOs:1247-1464, by using techniques known in the art, for example, phage display, bacterial display, yeast display, mammalian display, ribosome display, mRNA display, and cDNA display, or any other methods known in the art, such as those described in U.S. Patent Publication No. 2019/0144556.

A human TNFR2 antagonist mAb, when added to standard Treg expansion culture conditions, inhibits the expansion of Tregs and reduces their suppressive activity (see, e.g., Zou et al. (2018) Front. Immunol. 9:594). Two potent, dominant anti-human TNFR2 antagonistic antibodies that outcompete TNF (the natural agonist of TNFR2), inhibit TNF-induced in vitro expansion of human Tregs, and can induce the death of Tregs in vitro. The TNFR2 antagonists bind TNFR2 specifically through the F(ab) region, independently of the Fc region or the crosslinking of antibodies, and block the binding of TNF to TNFR2 by binding to the antiparallel dimers of TNFR2. As a result, TNF-induced activation of NF-κB pathways in Tregs is inhibited, and conversion of transmembrane TNFR2 (tmTNFR2) to soluble TNFR2 (sTNFR2) is suppressed. Tregs isolated from ovarian cancer tissues were found to be more sensitive to TNFR2 antagonist mAb-induced cell death, due to higher levels of TNFR2 expression on tumor-infiltrating Tregs. The TNFR2 antagonists also induced the death of TNFR2⁺ OVCAR3 (ovarian cancer) tumor cells, which also express TNFR2. These results indicate the therapeutic potential for TNFR2 antagonists in the treatment of tumors, by targeting tumor-infiltrating Tregs as well as tumor cells (see, e.g., Zou et al. (2018) Front. Immunol. 9:594; Torrey et al. (2017) Sci. Signal. 10:eaaf8608).

In addition to anti-TNFR2 antagonistic mAbs, small molecules can inhibit TNFR2. For example, thalidomide is a small molecule synthetic glutamic acid derivative with immunomodulatory and anti-inflammatory properties; thalidomide and its structural analogs, lenalidomide and pomalidomide, are classified as immunomodulatory drugs. Thalidomide and its analogs inhibit TNF synthesis by downregulating NF-κB, destroying TNF mRNA, and targeting reactive oxygen species and α1-acid glycoprotein, and also, inhibit surface expression of TNFR2 on T cells by inhibiting intracellular TNFR2 transport to the cell surface. It has been shown that thalidomide reduces the number and function of Tregs in patients with chronic lymphocytic leukemia, and, in patients with acute myeloid leukemia, combination therapy with lenalidomide and azacitidine downregulates TNFR2 expression on CD4⁺ T cells and reduces the number of TNFR2⁺ Tregs, enhancing effector immune function. In patients with multiple myeloma, however, treatment with thalidomide and its analogs increased the number of Tregs, likely due to the elevated serum levels of TNF following treatment, indicating that the effects of thalidomide on TNFR2⁺ Tregs is disease specific (see, e.g., Zou et al. (2018)Front. Immunol. 9:594).

Another small molecule inhibitor of TNFR2 is panobinostat, a histone deacetylase inhibitor that can reduce FoxP3 expression and inhibit the suppressive activity of Tregs. Combination therapy with panobinostat and azacitidine reduces the numbers of TNFR2⁺ Tregs in the blood and bone marrow of patients with acute myeloid leukemia, and the resulting increase in IFNγ and IL-2 production by effector T cells results in a therapeutic effect in these patients (see, e.g., Zou et al. (2018) Front. Immunol. 9:594). Cyclophosphamide, a DNA alkylating agent commonly used as a cytotoxic chemotherapeutic in cancer treatment, can inhibit immunosuppressive function of Tregs at low doses, and depletes the maximally suppressive Tregs in mice bearing PROb colon cancer following the administration of a single dose, resulting in the activation of anti-tumor immune responses. In a mouse model of mesothelioma, cyclophosphamide treatment depleted TNFR2^{hi} Tregs. The combination of cyclophosphamide with etanercept inhibited the growth of established CT26 tumors in mice, by blocking TNF-TNFR2 interaction and eliminating TNFR2-expressing Treg activity (see, e.g., Zou et al. (2018) Front. Immunol. 9:594). Triptolide, an immunosuppressive molecule isolated from the Chinese herb Tripterygium wilfordii, inhibits TNF and TNFR2 expression in the colon of a mouse colitis model, and also, decreases the number of Tregs and inhibits tumor growth in mice with melanoma (see, e.g., Zou et al. (2018) Front. Immunol. 9:594).

### F. Selective Targeting of the TNFR1 and/or TNFR1 and TNFR2 Axis

As described herein, existing anti-TNF therapies, which block TNF and inhibit its signaling via TNFR1 and TNFR2, are limited in therapeutic efficacy, tolerability, and safety. Anti-TNF therapies ameliorate RA and other autoimmune and inflammatory diseases and conditions by preventing TNF signaling through TNFR1, and abrogating apoptotic and inflammatory pathways. These anti-TNF therapies, however, also block the beneficial effects of TNFR2 signaling, including the protective, pro-survival, regeneration-promoting and anti-inflammatory signaling pathways, as well as the TNFR2-associated expansion of immunosuppressive Tregs, resulting in serious, sometimes fatal, side effects, including serious infections. Other side effects associated with the use of TNF blocking therapies include congestive heart failure, liver injury, demyelinating disease/CNS disorders, lupus, psoriasis, sarcoidosis, and an increased susceptibility to the development of additional autoimmune diseases, as well as cancers, including lymphomas and solid malignancies. Anti-TNF therapies have failed in the treatment of demyelinating and neurodegenerative diseases, and can exacerbate disease symptoms.

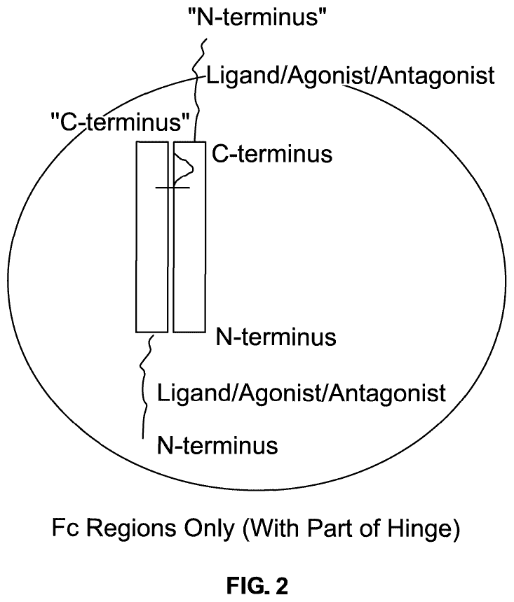

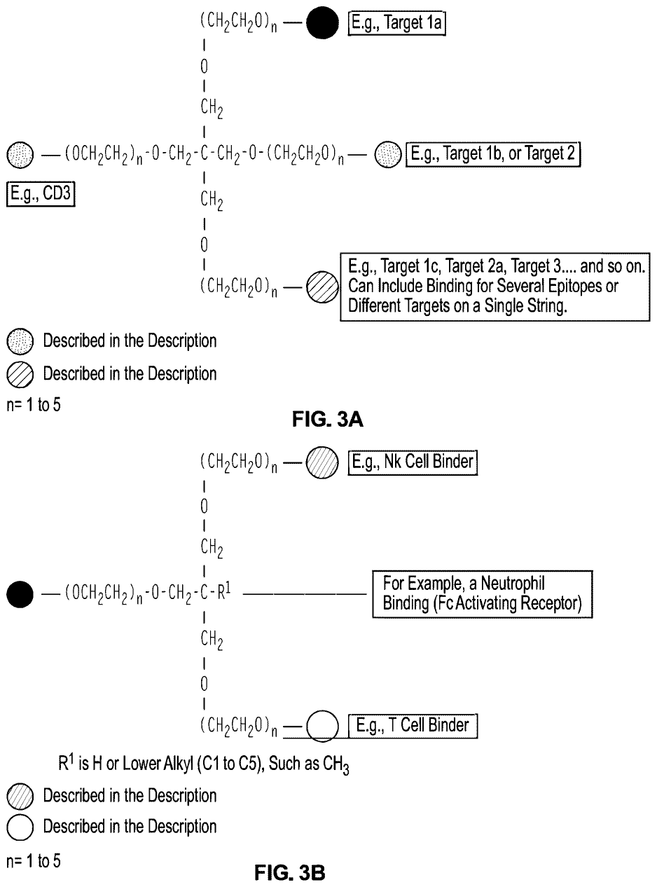

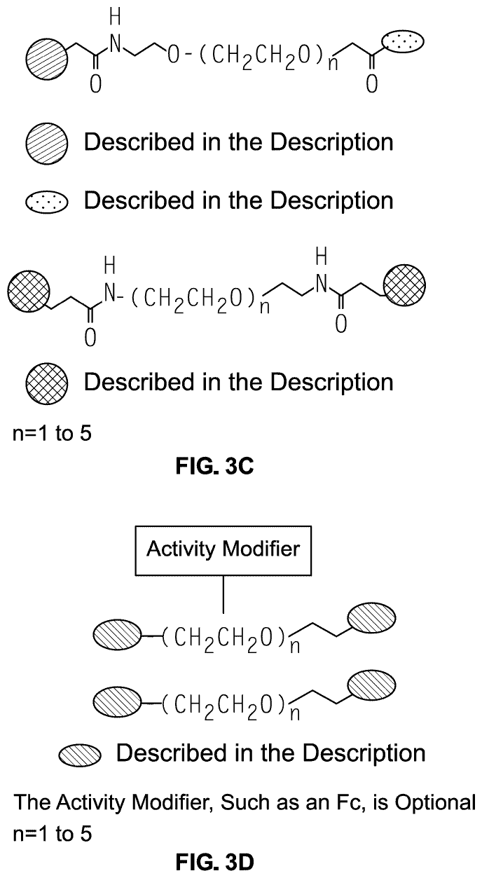

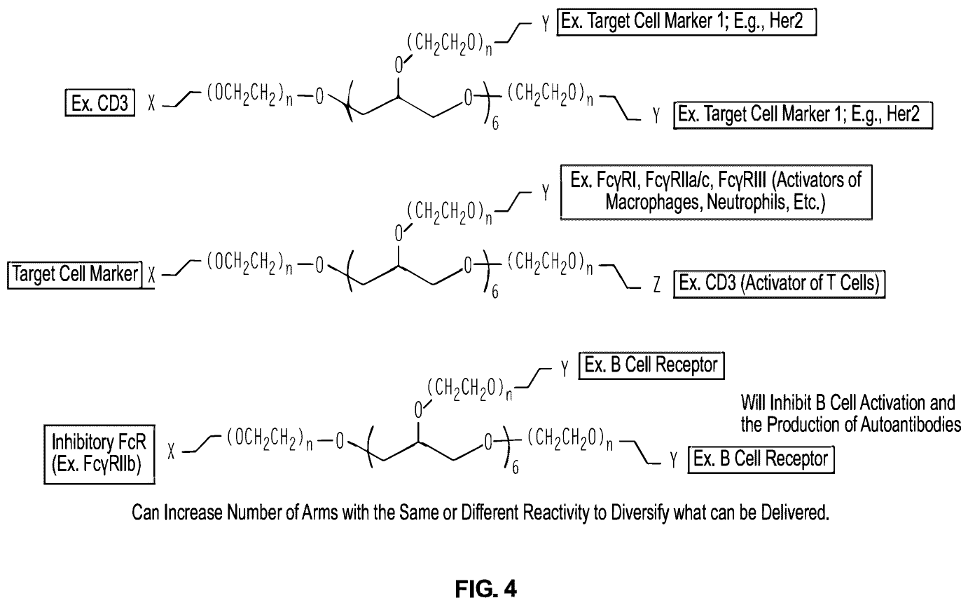

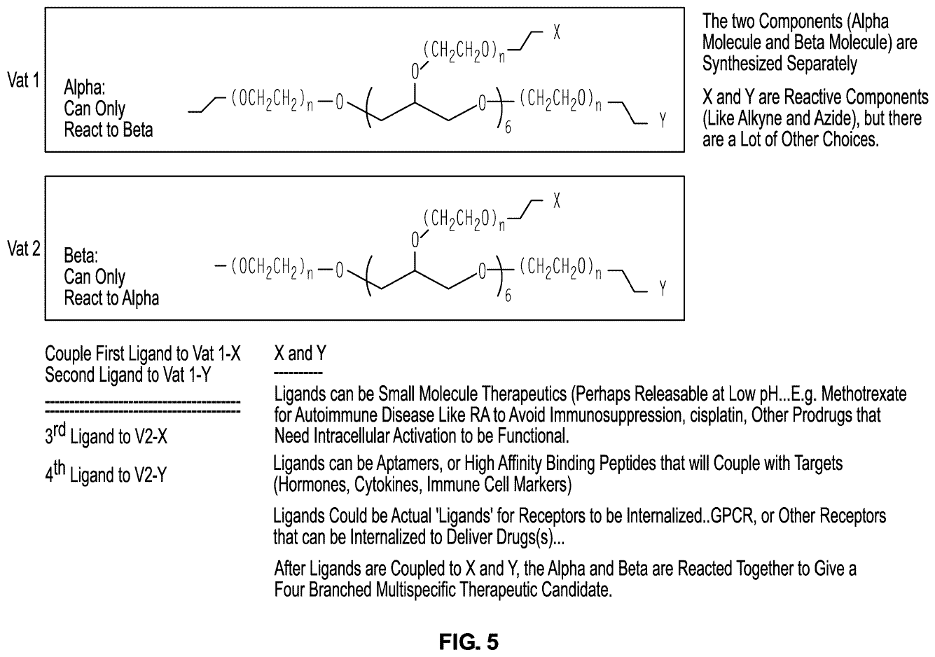

Provided herein are constructs, including TNFR1 antagonist constructs, multi-specific, such as bi-specific, TNFR1 antagonist/TNFR2 agonist constructs, and nucleic acids and methods for the selective inhibition of TNF signaling via TNFR1 (see, e.g., FIG. 2, which depicts an exemplary bi-specific construct). Also provided are constructs and methods for selective inhibition of TNF signaling via TNFR1, including while maintaining or enhancing TNFR2 signaling. These constructs and methods provide improved therapeutic approaches for the treatment of diseases and disorders of the TNF/TNFR1 axis. These therapeutic approaches include, but are not limited to treatments of autoimmune, chronic inflammatory, neurodegenerative, and demyelinating, diseases, disorders and conditions, and cancer, which also has an inflammatory component. As described herein, concomitant or sequential selective agonism of TNFR2 with TNFR1 antagonism has therapeutic effects, and can enhance the therapeutic index of selective TNFR1 antagonists, by activating desirable signaling pathways, such as anti-inflammatory pathways and NF-κB pathways that control cell survival and proliferation, and by inducing the expansion of immunosuppressive Tregs that remove excess autoreactive/effector T cells that result in tissue destruction, from the autoimmune microenvironment. As with all embodiments herein the TNFR1 antagonists are selected or designed or identified so that they do no exhibit TNFR1 agonist activity, including in bivalent or other multivalent configurations.

Sections 1 and 2 describe methods that target each of TNFR1 and TNFR2; section 3 provides an overview of constructs provided herein that solve the problems of prior approaches, particularly those that targeted TNFR1; and Section 4 describes the structure and components of constructs provided herein.

1. Selective Blockade of TNFR1 with TNFR1 antagonists

The use of multivalent agents, such as antibodies against TNFR1, however, is not feasible. The TNF trimer binds to three TNFR1 chains as a preligand assembly complex, mediated by the preligand assembly domains (plads) of each monomeric TNFR. This differs from most receptor systems where ligand binding is required before clusters form on the surface of the cell. The TNF receptors are single transmembrane glycoproteins with about 28% homology mostly in their extracellular domain with both receptors containing four tandemly repeated cysteine rich motifs. Their intracellular sequences are largely unrelated with almost no homology between each other, and early work indicated delineation of their signaling functions (Grell et al. (1994) J. Immol. 153(5):1963-72). They contain several motifs with known functional significance. Each of TNFR1 and TNFR2 contains an extracellular pre-ligand-binding assembly domain (PLAD) domain (distinct from ligand binding regions) that precomplexes receptors. Conformational changes are induced when the trimeric TNF ligand binds to the TNFR trimer in the cell membrane, resulting in signal activation (MacEwan (2002) Br J Pharmacol. 135(4):855-875; and Lo et al. (2019) Sci Signal. 12(592):eaav5637).

As a result, antibodies and other multivalent agents that bind to TNFR1 likely are not suitable for use as antagonists, because they can cause super-clustering leading to activation of TNFR signaling. Monovalent antagonists, such as single domain antibodies (dAbs or sdAbs), nanobodies (Nbs; camelid single domain antibodies), scFv fragments, and Fab fragments, on the other hand, bind to one TNFR1 molecule, and do not induce cross-linking or clustering of the receptor on cell surfaces, abrogating any activation of TNFR1 signaling. Monovalent antagonists can bind to domain 1, 2, 3 or 4, or to an epitope spanning multiple domains, of the TNFR1 extracellular domain (see, e.g., U.S. Pat. Nos. 9,028,817 and 9,028,822), but these existing antagonists were ineffective therapeutics. Among a variety of problems were the short serum-half-lives, and immunogenicity, and other problems. Selective blockade of TNFR1 can be achieved with TNFR1 antagonists with properties described and provided herein. In all embodiments described above and below, the TNFR1 antagonists are those that also do not have TNFR1 agonist activity, such as when in a bivalent construct. These include TNFR1 antagonists that bind to the same epitope as the constructs, such as the EN1206 constructs, that contain the dAb of SEQ ID NO:59.

2. Selective Activation of TNFR2 with TNFR2 agonists

As described herein, selective activation of TNFR2 can be achieved using TNFR2-specific agonists, which can include, for example, TNFR2 agonistic antibodies and antigen-binding fragments thereof, and TNFR2-selective TNF muteins and fusion proteins thereof. Antigen-binding fragments of antibodies that bind to the first and/or second epitope of human TNFR2 can be used. The first epitope of TNFR2 includes amino acid residues 48-67 of SEQ ID NO:4, and the second epitope includes position 135 of SEQ ID NO:4, including, for example, residues 128-147, 130-149, 135-147, or 135-153, of SEQ ID NO:4 (see, e.g., International Application Publication No. WO 2014/124134; and U.S. Pat. No. 9,821,010). Other epitopes on TNFR2 have been identified and can be used to design antigen-binding fragments with TNFR2-selectivity, as discussed below.

In contrast to the antagonism of TNFR1, to agonize TNFR2, dimeric and trimeric molecules are used that mimic the action of membrane-bound TNF, which is the primary ligand that activates TNFR2. As such, TNFR2 agonists include TNFR2-selective TNF muteins and antibody fragments. Exemplary are TNF mutein and antibody fragments that fused with multimerization domains, particularly dimerization or trimerization domains, as discussed below. For extending half-lives of these molecules they can be associated with or coupled to polyethylene glycol with or without cleavable linkers (see, e.g., Santi et al. (2012) Proc. Natl. Acad. Sci. U.S.A. 109:6211-6216), or fused or bound to half-life extender proteins or peptides, such as human serum albumin (with or without FcRn optimization, and with or without itself being PEGylated); and ADCC-inactivated/FcRn optimized Fe domains of antibodies with or without PEGylation (reviewed, for example, in Strohl (2015) BioDrugs 29(4):215-239). Half-life extenders, include, for example, PEGylation, modification of glycosylation, sialyation, PASylation (polymers of PAS amino acids about 100-200 residues in length), ELPylation (see, e.g., Floss et al. (2010) J. Trends Biotechnol. 28(1):37-45), HAPylation (glycine homopolymer), fusion to human serum albumin, fusion to GLK, fusion to CTP, GLP fusion, fusion to the constant fragment (Fc) domain of a human immunoglobulin (IgG), fusion to transferrin, fusion to non-structured polypeptides such as XTEN (also referred to as rPEG, genetic fusion of non-exact repeat peptide sequence, containing A, E, G, P, S, and T, see, e.g., Schellenberger et al. (2009) Nat Biotechnol. 27(12):1186-90), and other such modifications and fusions that increase size, increase hydrodynamic radius, alter charge, or target to receptors for recycling rather than clearance, and combinations of such modifications. Particular examples of extenders of half-life are discussed and exemplified in detail below.

3. TNFR1 antagonist constructs, TNFR2 agonist constructs; Multi-Specific, Including Bi-Specific, TNFR1 Antagonist and TNFR2 Agonist Constructs

Thus, provided herein are constructs for inhibiting TNFR1 signaling/activity and/or inhibiting TNFR1 and for agonizing TNFR2. Included among the constructs provided herein are constructs, discussed below, that are multi-specific, such as bi-specific that inhibit TNFR1 signaling and agonize TNFR2. Care is taken in designing these constructs, since bispecific antagonists TNFR1 or TNFR2 can inhibit the ability of TNF to induce activating changes in conformation of the resting trimeric TNFR, thus preventing its signaling. Other multimeric molecules risk the aggregation of receptors, thus forcing the TNFR to signal for cellular inflammation and apoptosis. Multi-specific constructs herein generally target different receptors, such as each of TNFR1 and TNFR2. By inhibiting TNFR1 signaling, and advantageously agonizing TNFR2 activity, this provides improved treatments of diseases, conditions, and disorders in which TNF is involved.

Among the constructs provided herein are TNFR1 antagonist constructs. These include fusion protein constructs, such as TNFR1 antagonist-Fc fusion constructs. As described herein, and exemplified in the Examples, TNFR1 antagonists that specifically target TNFR1, without antagonizing or without substantially antagonizing TNFR2, or that include or exhibit TNFR2 agonist activity can be selected, generated, or designed. The TNFR1 antagonist constructs improve the therapeutic efficacy and safety of prior TNFR1 antagonists, including monovalent antagonists, such as the dAbs, scFvs and Fabs.

Also provided are selective TNFR2 agonist constructs, such as TNFR2-Fc fusion constructs that improve the therapeutic efficacy of prior TNFR2 agonists. For example, as shown herein, the half-life of the Fc fusion constructs increases the half-life of prior TNFR1 antagonists or TNFR2 agonists, which, for example, reduces the frequency of dosing, improves patient compliance, and improves the therapeutic index. Also provided are selective TNFR2 agonist constructs, such as TNFR2-Fc fusion constructs that improve the therapeutic efficacy of prior TNFR2 agonists. For example, as shown herein, the half-life of the Fc fusion constructs increases the half-life of prior TNFR1 antagonists or TNFR2 agonists, which, for example, reduces the frequency of dosing, improves patient compliance, and improves the therapeutic index. Alternative candidate half-life extenders including PEGylating and fusion to peptides, are discussed above, and exemplary extenders are detailed below (reviewed in, Strohl (2015) BioDrugs 29(4):215-239, see also, Tan et al. (2018) Current Pharmaceutical Design 24:4932-4946), but also includes PEGylation using linear or branched PEG (see, e.g., Swierczewska et al. (2015) Expert Opin Emerg Drugs 20(4):531-536).

The TNFR1 antagonist constructs include an optional linker and an optional activity modifier. They can be assembled in any order. The structure of TNFR1 antagonist constructs can be represented by the formulae 1:

(TNFR1 inhibitor)ₙ-linkerₚ-(activity modifier)_{q}, formula 1a, or

(activity modifier)g-linkerₚ-(TNFR1 inhibitor)ₙ, formula 1b, where:

each of n and q is an integer, and each is independently 1, 2, or 3; p is 0, 1, 2 or 3; and an activity modifier is a moiety, such as a polypeptide, such as albumin, or an Fc that is modified to have reduced or no ADCC activity, that increases serum half-life of the TNFR1 inhibitor; and the TNFR1 inhibitor is a molecule, such as a polypeptide or small drug molecule that binds to TNFR1 and inhibits its activity. The activity modifier is not a human serum albumin antibody or an unmodified Fc. Also provided are the TNFR2 agonists of formula 3: (TNFR2 agonist)ₙ-linkerₚ-(activity modifier)_{q}, where n, p and q, the linker, and the activity modifier, are as set forth for formula 1.

Also provided are multi-specific, including bi-specific, constructs that contain an TNFR1 antagonist (a TNFR1 inhibitor) and an TNFR2 agonist, linked directly or via a linker. Such constructs can include a TNFR1 antagonist of the above formula or can have a structure as set forth in formula 2 below. The bi-specific and multi-specific constructs selectively inhibit inflammatory and deleterious TNFR1 signaling, enhance protective and anti-inflammatory TNFR2 signaling. They include moieties that provide for advantageous pharmacokinetic properties, including increased serum half-life and stability, and reduced peripheral clearance, compared with prior TNFR1 antagonists and TNFR2 agonists.

The structure of the multi-specific, such as, bi-specific, molecules/constructs provided herein is represented by the following formula (Formula 2):

(TNFR1 inhibitor)ₙ-(activity modifier)ᵣ₁-Linker (L)ₚ-(activity modifier)ᵣ₂-(TNFR2 agonist)_{q},

where n=1, 2, or 3, p=1, 2, or 3, r1 and r2 each independently=0, 1, 2, and q=0, 1 or 2.

As with formulae 1, the order of components can vary. The linker can contain a plurality of components, such as a GS linker, a polymeric moiety, such as a PEG, or other such linker, or a hinge region, or other combinations of components, and the activity modifier is a moiety that modify the activity of the construct, such as an Fc region, or a modified Fc region, or a polypeptide increases half-life, or resistance to endogenous inhibitors. The components of formulae 1 and 2 can be polypeptides or can contain other molecules, such as small drugs that specifically bind or a chemical linker, or a non-peptidic activity modifier. Examples of each component are described below.

### 4. Components of the TNFR1 Antagonist Constructs

Description of and examples of the constructs, and each component of the constructs provided herein are described in the sections below. Exemplary forms of each construct are depicted and described by formulae 1 and 2, above, and 3 and 4, below.

a. TNFR1 Inhibitor Moiety (TNFR1 Antagonist)

The TNFR1 inhibitor moiety in formula 1, above, and in the multi-specific molecules/constructs (formula 2, above) provided herein is any molecule, including a polypeptide or small molecule, that inhibits TNFR1 signaling. This includes a TNFR1 inhibitor that selectively inhibits TNFR1 signaling, without inhibiting TNFR2 signaling.

In order to avoid receptor clustering, which agonizes TNFR1, the TNFR1 antagonist construct generally is monomeric/monovalent. The TNFR1 antagonist inhibitor component of the construct can be one that is known to have TNFR1 antagonist activity, or can be identified, such as by selecting from a library, such as a phage library, an antibody library, or an aptamer library. Among the TNFR1 inhibitor moieties are those that are modified or selected to have increased specificity or affinity for TNFR1, and, have no or little (such that the adverse side effects from such activity are less than grade 2, and generally grade 1 or less based on the NCI Common Terminology Criteria for Adverse Events (CTCAE) grading system) agonist activity for TNFR1, and optionally also have agonist activity for TNFR2. In those instances, the TNFR1 inhibitor moiety can be provided as a single chain antibody or in any of the other forms described herein, including, such as linked to a half-life extender, such as any described above and below, such as a modified Fc region or Fc dimer, or to another moiety or moieties that increase(s) serum half-life.

For example, as provided herein, the TNFR1 inhibitor component of the TNFR1 antagonist construct can be or can include a human domain antibody (dAb) that specifically binds to TNFR1. The dAb can contain a variable-region heavy chain (V_{H}) or light chain (V_{L}) domain. dAbs for use herein include, for example, dAbs designated DOM1 h-574-208 (SEQ ID NO:54) (from DMS5541; see, SEQ ID NO:38), GSK1995057 (see, SEQ ID NO:55) and GSK2862277 (see, SEQ ID NO:56), as well as the dAbs set forth in any of SEQ ID NOs: 57-672 (see, e.g., U.S. Pat. Nos. 9,028,817 and 9,028,822; U.S. Publication Nos.: 2006/0083747, 2010/0034831, and 2012/0107330; and International Application Publication Nos.: WO 2004/058820, WO 2004/081026, WO 2005/035572, WO 2006/038027, WO 2007/049017, WO 2008/149144, WO 2008/149148, WO 2010/094720, WO 2011/051217, WO 2011/006914, WO 2012/172070, WO 2012/104322, and WO 2015/104322, and other related family member applications and patents; see, also Enever et al., (2015) Protein Engineering, Design & Selection 28(3):59-66, which provides sequences and discussion of various dAbs). Provided are Vhh dAbs that contain a heavy chain. These dAbs can be linked directly or indirectly to a moiety, such as Fc or HSA, that increases serum half-life, and also that can impart other properties or activities to a construct.

The anti-TNFR1 inhibitor component can be or include a nanobody. Exemplary of these are (Nbs) Nb 70 and/or Nb 96 (see, SEQ ID NOs: 683 and 684, respectively). These dAbs and Nbs are surveyed for immunogenicity, and, if needed, using molecular modeling and mutagenesis, are modified to remove predicted immunogenic sequences. Immunogenic sequences can be eliminated by standard methods known in the art. For example, identify the potentially antigenic peptides, and make of conservative replacements of each amino acid to identify those that are not antigenic and that retain activity. Other methods are known (see, e.g., Schubert et al. (2018)PLoS Comput Biol. 14(3):e1005983), which describes a method for de-immunizing proteins). They also are modified so that in any bivalent format they do not have TNFR1 agonist activity.

Thus, for example, the TNFR1 antagonist dAb portion, can be the dAb set forth in any of SEQ ID NOs: 54-672, or a dAb with about or at least about 85%, 90%, 95%, 98%, 99%, or greater, sequence identity to a dAb set forth in any of SEQ ID NOs: 54-672, or a TNFR1 antagonist dAb known to those of skill in the art.

Other TNFR1 antagonists include, for example, antigen-binding antibody fragments. For example, the TNFR1 antagonist can be a Fab fragment, Fab′ fragment, single-chain Fv (scFv), disulfide-linked Fv (dsFv), Fd fragment, Fd′ fragment, single-chain Fab (scFab), hsFv (helix-stabilized Fv), a free light chain, or antigen-binding fragments of any of the above. It also can include linkers, such as GS linkers within the construct, for example, to increase flexibility.

For example, the TNFR1 inhibitor portion of the antagonist can contain antigen-binding fragments from the TNFR1 antagonistic antibody designated ATROSAB. The fragments include one or more (or all) of the heavy chain or light chain CDRs of ATROSAB, or CDRs that exhibits at least 85%, 90%, 95% or more sequence identity thereto (e. g., 85%, 90%, 95%, 96%, 97%, 98%, 99% or more sequence identity). The TNFR1 antagonist can contain the V_{H} (residues 1-115 of SEQ ID NO:31) and/or V_{L} (residues 1-113 of SEQ ID NO:32) of ATROSAB, or a V_{H} or V_{L} containing at least 85%, 90%, 95%, or more, sequence identity to the V_{H} or V_{L} of ATROSAB. For example, it can contain a dAb derived from ATROSAB. The TNFR1 antagonist can contain other monovalent antibody fragments of ATROSAB, including, for example, Fab or scFv fragments, such as the ATROSAB Fab (FabATR) light and heavy chains set forth in SEQ ID NOs: 679 and 680, respectively, or the ATROSAB scFv (scFv IZI06.1) set forth in SEQ ID NO:673. For example, the scFv contains the V_{H} domain, corresponding to residues 1-115 of the ATROSAB heavy chain (see, SEQ ID NO:31), linked by a short peptide linker (e.g., GGGGSGGGGSGGSAQ, as in SEQ ID NO:673, or a linker set forth in any of SEQ ID NOs:813-834) to the V_{L} domain, corresponding to residues 1-113 of the ATROSAB light chain (see, SEQ ID NO:32). The TNFR1 antagonist can contain variants of the ATROSAB scFV with increased affinity or selectivity or both for TNFR1, including scFv IG11, which includes or has the sequence set forth in SEQ ID NO:674, scFv T12B, containing the sequence set forth in SEQ ID NO:675, or scFv 13.7, containing the sequence set forth in SEQ ID NO:676, or variants containing at least 90% sequence identity to the sequences of scFv IG11, scFv T12B, and scFv 13.7. The TNFR1 antagonist also can include the sequence of amino acid residues from the Fab 13.7 light and heavy chains (derived from scFV 13.7), as set forth in SEQ ID NOs: 681 and 682, respectively.

TNFR1 inhibitors in the TNFR1 antagonist construct also include TNF variants (muteins) that bind to TNFR1 to reduce or inhibit signaling. These include, for example, TNF variants (muteins), such as, but not limited to, TNF variants containing one or more of the mutations L29S, L29G, L29Y, R31E, R31N, R32Y, R32W, S86T, L29S/R32W, L29S/S86T, R32W/S86T, L29S/R32W/S86T, R31N/R32T, R31E/S86T, R31N/R32T/S86T, and E146R, with reference to SEQ ID NO:2, which impart selectivity to TNFR1. The TNFR1 antagonist can contain, for example, the TNFR1-selective antagonistic TNF mutein derived from the mutein designated XPro1595 (see, SEQ ID NO:701). XPro1595 contains the mutations ViM, R31C, C69V, Y87H, C101A and A1456R, with reference to SEQ ID NO:2. Other exemplary TNFR1-selective antagonistic TNF muteins are derived from XENP345 (see, SEQ ID NO:702), which contains the mutations I97T/A145R, with reference to SEQ ID NO:2; and the TNFR1-selective antagonistic TNF mutein designated R1antTNF (see, SEQ ID NO:703), which contains the mutations A84S, V85T, S86T, Y87H, Q88N and T89Q, with reference to SEQ ID NO:2. TNFR1 inhibitors to be used in the TNFR1 antagonists also include small molecule inhibitors that can be chemically conjugated to a linker.

As described herein, see e.g., the Examples, the TNFR1 inhibitor (antagonist) moiety can be modified to improve its specificity/selectivity for TNFR1, and also, optionally can be modified to have TNFR2 agonist activity. TNF binds to TNFR1 with low pM affinity (K_{d} 19 pM); in general the antagonists herein have at least the same affinity as TNF, unless its activity is due to ‘locking’ the receptor in an inactive conformation, then it is not necessary since the receptors become locked. TNFR1 antagonist constructs provided herein, include those that specifically bind to TNFR1 with a K_{D} value of less than or less than about 100 nM (e.g., less than or equal to: 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 5 nM, 4 nM, 3 nM, 2 nM, or 1 nM). In certain embodiments, the TNFR1 antagonists specifically bind to TNFR1 with a K_{D} value of less than 1 nM (e.g., less than or equal to: 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, 160 pM, 150 pM, 140 pM, 130 pM, 120 pM, 110 pM, 100 pM, 90 pM, 80 pM, 70 pM, 60 pM, 50 pM, 40 pM, 30 pM, 20 pM, 10 pM, 5 pM, or 1 pM).

The TNFR1 antagonist constructs provided herein also are selected or designed so that they lack or have reduced binding for other TNFR superfamily members. For example, they are assessed to identify those that do not specifically bind to another TNFR superfamily member, such as TNFR2, using any suitable in vitro binding assay. Assays include, for example, ELISA-based methods. For example, the TNFR1 antagonist constructs can specifically bind to human TNFR1 or a TNFR1-derived peptide, with an affinity that is greater than the affinity for another family member or corresponding peptide thereof. The increased affinity is, for example, at least or at least about 5-fold greater (e.g., at least or equal to 5-fold greater, 6-fold greater, 7-fold greater, 8-fold greater, 9-fold greater, 10-fold greater, 20-fold greater, 30-fold greater, 40-fold greater, 50-fold greater, 60-fold greater, 70-fold greater, 80-fold greater, 90-fold greater, 100-fold greater, 200-fold greater, 300-fold greater, 400-fold greater, 500-fold greater, 600-fold greater, 700-fold greater, 800-fold greater, 900-fold greater, 1,000-fold greater, 2,000-fold greater, 3,000-fold greater, 4,000-fold greater, 5,000-fold greater, 6,000-fold greater, 7,000-fold greater, 8,000-fold greater, 9,000-fold greater, 10,000-fold greater, or more), than the affinity of the TNFR1 antagonist for another TNFR superfamily member, such as TNFR2.

Among the TNFR1 antagonist constructs provided herein are those that exhibit high kₒₙ and low k_{off} values upon interaction with TNFR1, consistent with high-affinity receptor binding. For example, the TNFR1 antagonist constructs provided herein can exhibit kₒₙ values in the presence of TNFR1 of greater than or equal to, or greater than, about 10⁴ M⁻¹s⁻¹ (e.g., greater than or equal to 1.0×10⁴ M⁻¹s⁻¹, 1.5×10⁴ M⁻¹s⁻¹, 2.0×10⁴ M⁻¹s1, 2.5×10⁴ M⁻¹s⁻¹, 3.0×10⁴M⁻¹s1, 3.5×10⁴ M⁻¹s⁻¹, 4.0×10⁴ M-s⁻¹, 4.5×10⁴ M⁻¹s¹, 5.0×10⁴ M⁻¹s¹, 5.5×10⁴ M⁻¹s¹, 6.0×10⁴ M⁻¹s¹, 6.5×10⁴ M⁻¹s1, 7.0×10⁴ M⁻¹s1, 7.5×10⁴ M⁻¹s⁻¹, 8.0×10⁴ M⁻¹s1, 8.5×10⁴ M⁻¹s⁻¹, 9.0×10⁴ M⁻¹s⁻¹ 9.5×10⁴ M⁻¹s⁻¹, 1.0×10⁵ M⁻¹s¹, 1.5×10⁵ M⁻¹s¹, 2.0×10⁵ M⁻¹s¹, 2.5×10⁵ M⁻¹s¹, 3.0×10⁵ s⁻¹, 3.5×10⁵ M⁻¹s¹, 4.0×10⁵ M⁻¹s⁻¹, 4.5×10⁵ M⁻¹s¹, 5.0×10⁵ M⁻¹s¹, 5.5×10⁵ M⁻¹s⁻¹, 6.0×10⁵ M⁻¹s1, 6.5×10⁵ M⁻¹s1, 7.0×10⁵ M⁻¹s⁻¹, 7.5×10⁵ M⁻¹s⁻¹, 8.0×10⁵ M⁻¹s⁻¹, 8.5×10⁵ M⁻¹s⁻¹, 9.0×10⁵ M⁻¹s⁻¹, 9.5×10⁵ M⁻¹s⁻¹, 1.0×106 M⁻¹s⁻¹). For example, the TNFR1 antagonists provided herein can exhibit k_{off} values, when complexed to TNFR1, of less than or equal to, or less than about 10⁻³ s⁻¹ (e.g., less than or less than about 1.0×10⁻³ s⁻¹, 9.5×10⁻⁴ s⁻¹, 9.0×10⁻⁴ s⁻¹, 8.5×10⁻⁴s⁻¹, 8.0×10⁻⁴ s⁻¹ 7.5×10⁻⁴ s⁻¹ 7.0×10⁻⁴ s⁻¹ 6.5×10⁻⁴s⁻¹ 6.0×10⁻⁴s⁻¹ 5.5×10⁻⁴ s⁻¹ 5.0×10⁻⁴s⁻¹ 4.5×10⁻⁴ s⁻¹ 4.0×10⁻⁴s⁻¹, 3.5×10⁻⁴ s⁻¹ 3.0×10⁻⁴s⁻¹, 2.5×10⁻⁴s⁻¹ 2.0×10⁻⁴ s⁻¹ 1.5×10⁻⁴ s⁻¹ 1.0×10⁻⁴s⁻¹ 9.5×10⁻⁵ s⁻¹ 9.0×10⁻⁵ s⁻¹ 8.5×10⁻⁵s⁻¹ 8.0×10⁻⁵s⁻¹ 7.5×10⁻⁵ s⁻¹ 7.0×10⁻⁵ s⁻¹ 6.5×10⁻⁵s⁻¹ 6.0×10⁻⁵s⁻¹ 5.5×10⁻⁵ s⁻¹ 5.0×10⁻⁵ s⁻¹ 4.5×10⁻⁵ s⁻¹ 4.0×10⁻⁵ s⁻¹, 3.5×10⁻⁵ s⁻¹, 3.0×10⁻⁵ s⁻¹, 2.5×10⁻⁵s⁻¹, 2.0×10⁻⁵s⁻¹, 1.5×10⁻⁵s⁻¹, or 1.0×10⁻⁵s⁻¹).

The C-terminus of the TNFR1 antagonist (TNFR1 inhibitor portion of the construct of formula 1 and also formula 2), such as any of the TNFR1 antagonist constructs described herein, can be linked, directly, or more generally via a linker or combination of linker elements, to an activity modifier, or fused with the N-terminus of an TNFR2 agonist (or to a small molecule TNFR2 agonist) via one or more linkers, as discussed below and elsewhere herein. Alternatively, the N-terminus of the TNFR1 inhibitor moiety can be fused to the C-terminus of the TNFR2 agonist, or the C-terminus of the TNFR1 inhibitor moiety (or to a small molecule TNFR2 agonist) can be fused directly or via linker to the activity modifier or to a linker.

The linkers (L), discussed in more detail below, are any that improve pharmacological properties, including increasing stability and flexibility and decreasing steric hindrance, and optionally conferring additional properties on the constructs. The linkers can include more than one component, where each component confers a particular property, For example, the TNFR1 antagonists can include any one or more of an Ig Fc region, and/or an antibody hinge region, and/or a short peptide linker, such as a glycine-serine linker. The Fc regions are modified, for example, to eliminate or reduce ADCC activity, and/or to alter receptor binding, and/or for other such activities and properties. Linkers, as discussed below, also include chemical linkers. For example, in some embodiments, the linker is a poly(ethylene glycol) (PEG) molecule, or a branched PEG molecule, such as those whose molecular mass is at or about 30 kDa or more.

b. Linkers

The TNFR1 antagonist constructs optionally include linkers, as well as activity modifiers. The linkers have a variety of functions, including provision of additional or improved biological and pharmacological properties, and for structural purposes for linking different molecules. Exemplary linkers are Gly-Ser polypeptides, hinge regions (see, e.g., Tables 1-4 above, which set forth the sequences of various hinge regions, and combinations thereof).

Included are polypeptide linkers and also chemical linkers for chemical conjugation. Linker peptides are included as spaces between polypeptides, and can promote proper protein folding and stability of the polypeptides, improve protein expression, and enhance the bioactivity of the components of the constructs. Peptide linkers primarily are designed to be unstructured, flexible peptides. Linkers can be included as set forth in exemplary formulae 1-4, above. For example, in the bi-specific constructs provided, the components are fused via a linker (L) in an N-terminus to C-terminus, or C-terminus to N-terminus configuration. The linker generally is a peptide linker, including a polypeptide, such as an Fc region, alone, or in combination with one or more other linkers, including, for example, short peptide linkers, such as a glycine-serine (GS) linker, and/or the hinge region of an immunoglobulin (Ig). In embodiments herein, for example, the C-terminus of the TNFR1 antagonist is fused to the N-terminus of the peptide linker(s), and the C-terminus of the peptide linker(s) is fused with the N-terminus of the TNFR2 agonist. In other embodiments, the C-terminus of the TNFR2 agonist is fused to the N-terminus of the peptide linker(s), and the C-terminus of the peptide linker(s) is fused with the N-terminus of the TNFR1 antagonist. The linker provides increased molecular weight, increasing the stability and serum half-life, enhancing tissue retention, and reducing or decreasing peripheral elimination, thereby improving the therapeutic index of the molecule. The linker also increases the flexibility of the molecule, allowing each portion of the molecule to interact with its target antigen/epitope, such as TNFR1 and TNFR2, as provided herein. As discussed below and elsewhere herein, in embodiments where the linker contains an Fc region of an immunoglobulin, generally a modified Fc region, additional properties can be imparted, including, for example, neonatal Fc receptor (FcRn) recycling, which further increases serum stability and half-life, and/or the enhancement or elimination of immune effector functions.

i. Peptide Linkers

Linkers for fusion proteins are well known to those of skill in the art (see, e.g., Chen et al (2013) Adv. Drug. Deliv. Rev. 65:1357-1369, entitled “Fusion Protein Linkers: Property, Design and Functionality”). Linkers can be designed or can be from or based on linkers from naturally-occurring multi-domain proteins. Empirical linkers designed by researchers are generally classified into 3 categories, according to their structures: flexible linkers, rigid linkers, and in vivo cleavable linkers, which are used, for example, for delivering prodrugs that are activated by cleavage of the linker in situ.

Besides the role in linking the functional domains together (as in flexible and rigid linkers) or releasing the free functional domain in vivo (as in in vivo cleavable linkers), linkers also can improve properties of the linked moieties. These include, for example, improving biological activity, increasing expression yield, and achieving desirable pharmacokinetic profiles. Databases and methods for selecting linkers are known to those of skill in the art (see, e.g., George et al. (2002) “An analysis of protein domain linkers: their classification and role in protein folding,” Protein Eng. 15:871-879).

a) Flexible linkers

Flexible linkers are usually applied when the joined domains require a certain degree of movement or interaction. Flexible linkers are generally rich in small or polar amino acids such as Gly and Ser to provide good flexibility and solubility. They are suitable choices when certain movements or interactions (e.g., in an scFv) are required for fusion protein domains. In addition, although flexible linkers do not have rigid structures, they can serve as a passive linker to keep a distance between functional domains. The length of the flexible linkers can be adjusted to allow for proper folding or to achieve optimal biological activity of the fusion proteins.

Flexible linkers generally are composed of small, non-polar (e.g., Gly) or polar (e.g., Ser or Thr) amino acids as suggested by Argos (1990) J. Mol. Biol. 211(4):943-958. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties.

Exemplary flexible linkers are linkers that contain primarily or only stretches of Gly and Ser residues (“GS” linkers). An example is a flexible linker that has the sequence of (Gly-Gly-Gly-Gly-Ser)ₙ. By adjusting the copy number “n”, the length of this GS linker can be selected or chosen to achieve appropriate separation of the functional domains, or to maintain necessary inter-domain interactions. Flexible linkers are also rich in small or polar amino acids such as Gly and Ser, and also can contain additional amino acids, such as Thr and Ala, to maintain flexibility, as well as polar amino acids, such as Lys and Glu, to improve solubility.

To confer protease resistance and to increase the flexibility of the fusion protein, the SCDKTH hinge sequence and other hinge sequences can be replaced with, or preceded by, a short polypeptide linker. Exemplary of polypeptide linkers are (Gly-Ser)ₙ amino acid sequences (GS linkers), with some Glu or Lys residues dispersed throughout to increase solubility. For example, polypeptide linkers include, but are not limited to, (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)ₙ, where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS (see SEQ ID NOs: 816-827 for the GS linkers). The linker can be a poly-Gly peptide that is at least 2-18 residues in length, or longer, or a similar linker of the same length and flexibility. Exemplary polypeptide linkers in the molecules provided herein, include, but are not limited to (see SEQ ID NOs: 816-827 for Gly-Ser linkers): GSGS, GGGGS, or GGGGSGGGGSGGGGS, for example. Another linker that provides similar performance is a (GGGGS)₄ (SEQ ID NO:819) linker. Another Gly and Ser rich flexible linker is GSAGSAAGSGEF (SEQ ID NO:828). This linker has been shown to maintain good solubility in aqueous solutions. Linkers that contain only glycine can be used. For example (Gly)₆ (SEQ ID NO:1473) and (Gly)₈ (SEQ ID NO: 1474) linkers are known and shown to be stable against proteolytic enzymes digestion during protein purification from the expression organism.

Several other types of flexible linkers, including KESGSVSSEQLAQFRSLD (SEQ ID NO:829), and EGKSSGSGSESKST (SEQ ID NO:830). The Gly and Ser residues in the linker provide flexibility, and the Glu and Lys improve the solubility.

b) Rigid linkers

While flexible linkers have the advantage of connecting functional domains passively and permitting certain degree of movements, the lack of rigidity of these linkers can be limiting. Rigid linkers are chosen when the spatial separation of the domains is needed to preserve the stability or bioactivity of the fusion proteins. Rigid linkers exhibit relatively stiff structures by adopting a-helical structures or by containing multiple Pro residues. The length of the linkers can be easily adjusted by changing the copy number to achieve an optimal distance between domains.

Alpha helix-forming linkers with the sequence of (EAAAK)ₙ (SEQ ID NO:831) have been applied to the construction of many recombinant fusion proteins. An α-helical structure is rigid and stable, with intra-segment hydrogen bonds and a closely packed backbone. The stiff α-helical linkers can act as rigid spacers between protein domains. An example of a rigid linker is: A(EAAAK)ₙA (SEQ ID NO:832), wherein n=2-5. This linker displays an α-helical conformation, which was stabilized by the Glu-Lys⁺ salt bridges within segments. Another type of rigid linker has a Pro-rich sequence, (XP)ₙ, where X designates any amino acid, and is generally Ala, Lys, or Glu. The presence of Pro in non-helical linkers increases stiffness, and allows for effective separation of the protein domains. Examples of such linkers are 33-residue peptides containing repeating -Glu-Pro- and -Lys-Pro-.

Those of skill in the art can select from known linkers or design linkers. Desirable properties and requisites therefor are known. The following discussion summarizes some exemplary linkers (see, Chen et al. (2013) Adv. Drug. Deliv. Rev. 65:1357-1369), which provides details of flexible and rigid and cleavable linkers and that can be used). Flexible linkers are rich in small and/or hydrophilic amino acids such as Gly or Ser to provide the structural flexibility and have been used to connect functional domains that favor interdomain interactions or movements. Rigid linkers may be used where sufficient separation of protein domains is needed. Rigid linkers are designed or selected to be those that adopt α-helical structures or incorporate proline. Rigid linkers can keep protein moieties at a distance. Flexible and rigid linkers are stable in vivo, and do not allow the separation of joined proteins. Cleavable linkers permit the release of free functional domain in vivo via reduction or proteolytic cleavage. They generally are used for delivery of a prodrug to a target site.

An additional linker, such as between the TNFR1 antagonist, and the activity-modifying portion, such as the Fc portion, can be included; such linkers can contain, for example, all or a portion of the hinge sequence, sufficient to provide flexibility, of trastuzumab, including at least the residues SCDKTH (corresponding to residues 222-227 of SEQ ID NO:26), or all or a portion, containing a sufficient portion to provide flexibility, of the hinge region of nivolumab, with the sequence ESKYGPPCPPCP (corresponding to residues 212-223 of SEQ ID NO:29) or a sequence having at least 98% or 99% sequence identity thereto, or any other suitable antibody hinge region or sequence known in the art.

An Fc subunit, which is or is not a fusion protein, can be included (and can be modified to contain knobs-in-holes (see discussion below). It will assemble within the mammalian cell expression system to form a knobs-in-hole mediated Fc dimer to create an Fc dimer, which further increases the serum half-life and stability of the molecule. In certain embodiments, the second Fc subunit is fused with a TNFR1 antagonist creating a bivalent antibody-like structure. In other embodiments, only one Fc subunit is included (an Fc monomer).

ii. Chemical Linkers

In some embodiments, the linker is a chemical linker. These include linkers that are non-cleavable moieties, chemical cross-linking reagents, and polypeptide modifying agents, such as polymeric molecules, including PEGylation moieties. Chemical linkers are more amenable to creation of branched constructs and other structures that cannot be achieved with peptide linkers.

Exemplary linkers include non-cleavable linkers. Non-cleavable linkers include, for example, amide linkers and amide and ester linkages with succinate spacers (see, e.g., Dosio et al., (2010) Toxins 3:848-883). Exemplary chemical cross-linking linkers include, but are not limited to, SMCC (Succinimidyl-4-(N-maleimidomethyl)cyclohexane-1-carboxylate) and SIAB (Succinimidyl (4-iodoacetyl)aminobenzoate). SMCC is an amine-to-sulfhydryl crosslinker that contains NHS-ester and maleimide reactive groups at opposite ends of a medium-length cyclohexane-stabilized spacer arm. SIAB is a short, NHS-ester and iodoacetyl crosslinker for amine-to-sulfhydryl conjugation. Other exemplary cross-linking reagents include, but are not limited to, thioether linkers, chemically labile hydrazone linkers, 4-mercaptovaleric acid, BMPEO, BMPS, EMCS, GMBS, HBVS, LC-SMCC, MBS, MPBH, SBAP, SIA, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfonyl)benzoate), and bis-maleimide reagents, such as DTME, BMB, BMDB, BMH, BMOE, BM(PEO)₃, and BM(PEO)₄, which are commercially available (Pierce Biotechnology, Inc.). Bis-maleimide reagents allow the attachment of a free thiol group of a cysteine residue of an antibody to a thiol-containing targeted agent, or linker intermediate, in a sequential or concurrent fashion. Other thiol-reactive functional groups, besides maleimide, include iodoacetamide, bromoacetamide, vinyl pyridine, disulfide, pyridyl disulfide, isocyanate, and isothiocyanate. Other exemplary linkers and methods of use are well known to those of skill in the art, for example, the linkers and methods described in U.S. Patent Publication No. 2005/0276812, and in Ducry et al. (2010) Bioconjug. Chem. 21:5-13.

Linkers optionally can be substituted with groups that modulate properties, such as solubility and reactivity. For example, a sulfonate substituent can increase water solubility of the reagent and facilitate the coupling reaction of the linker reagent with an antibody or drug moiety, and/or facilitate coupling reactions. Linker reagents can also be obtained via commercial sources, such as Molecular Biosciences Inc. (Boulder, CO.), or synthesized in accordance with procedures described in Toki et al. (2002) J. Org. Chem. 67:1866-1872; U.S. Pat. No. 6,214,345; U.S. Publication Nos. 2003/130189, and 2003/096743; and International Application Publication Nos. WO 02/088172, WO 03/026577, WO 03/043583, and WO 04/032828. For example, linker reagents such as DOTA-maleimide (4-maleimidobutyramidobenzyl-DOTA) can be prepared by the reaction of aminobenzyl-DOTA with 4-maleimidobutyric acid (Fluka) activated with isopropylchloroformate (Aldrich), following the procedure of Axworthy et al. (2000) Proc. Natl. Acad. Sci. U.S.A. 97(4):1802-1807. DOTA-maleimide reagents react with the free cysteine amino acids of the cysteine engineered antibodies and provide a metal complexing ligand on the antibody (Lewis et al. (1998) Bioconj. Chem. 9:72-86). Chelating linker labelling reagents, such as DOTA-NHS (1,4,7,10-tetraazacyclododecane-1,4,7,10-tetraacetic acid mono (N-hydroxysuccinimide ester)), are commercially available (Macrocyclics, Dallas, TX.).

The linker can be a dendritic type linker for covalent attachment of more than one moiety through a branching, multifunctional linker moiety to an antibody (see, e.g., Sun et al. (2002) Bioorganic & Medicinal Chemistry Letters 12:2213-2215; Sun et al. (2003) Bioorganic & Medicinal Chemistry 11:1761-1768; King et al. (2002) Tetrahedron Letters 43:1987-1990). If an antibody bears only one reactive cysteine thiol group, a multitude of other moieties can be attached through a dendritic linker. Exemplary dendritic linker reagents are known (see, e.g., U.S. Patent Publication No. 2005/0276812).

Another example of a chemical linker (also can be an activity modifier for use in constructs herein) are PEG molecules, and branched PEG molecules, particularly those with a molecular weight of 30 kDa or more. A PEG linker provides for the introduction of multispecificity and bivalency (in the case of TNFR2 agonists where receptor clustering enhances signaling), and increases the molecular weight of the molecule, which increases in vivo serum half-life. PEG linkers also ameliorate difficulties in the re-engineering of antibodies, for example, by avoiding the introduction of non-natural structures that are degraded and cleared rapidly and/or cause immunogenicity.

c. Fc—Activity Modifiers

Among the components of constructs are portions or regions that modulate or alter the activity and/or pharmacological properties of the constructs (see formula 1 and 2 above). Exemplary of such are Fc regions, modified Fc regions, other multimerization domains, dimers of the Fc and modified Fc, can increase serum half-life. Activity modifiers can confer properties, such as, but not limited to, extending plasma half-life by decreasing access to proteases, decreasing renal filtration, and/or by altering the intracellular routing via receptor-mediated recycling; providing for absorption across epithelial bilayers by binding to receptors that undergo transcytosis; targeting in vivo sites that over-express or uniquely express specific receptors or antigens; and other properties, as exemplified in the discussion below, and also as known in the art.

As provided herein, the constructs can include, as an activity modifier, the Fc region of a human immunoglobulin, such as an IgG, for example, an IgG1 Fc (SEQ ID NO:10), an IgG2 Fc (SEQ ID NO:12), an IgG3 Fc (SEQ ID NO:14), or an IgG4 Fc (SEQ ID NO:16). In particular, the Fc is derived from an IgG1 or IgG4 antibody. For example, the linker can include an IgG1 kappa Fc region, such as the IgG1 Fc derived from trastuzumab, containing the C_{H}2 and C_{H}3 domains of the trastuzumab heavy chain (see, e.g., residues 234-450 of SEQ ID NO:26; see, also, SEQ ID NO:27). The Fe subunit in the bi-specific molecules provided herein also can be an IgG4 Fc, such as, for example, the IgG4 Fc derived from nivolumab (Opdivo®), containing the CH2 and C_{H}3 domains of the nivolumab heavy chain (see, e.g., residues 224-440 of SEQ ID NO:29; see, also, SEQ ID NO:30).

The Fc region can be mutated or modified, as discussed below, to eliminate, reduce, or enhance, immune effector functions, including, for example, any one or more of antibody-dependent cellular cytotoxicity (ADCC; also known as antibody-dependent cell-mediated cytotoxicity), antibody-dependent cell-mediated phagocytosis (ADCP), and complement-dependent cytotoxicity (CDC). In some embodiments herein, for example, where the construct is a bi-specific molecule is used to treat inflammatory and autoimmune diseases and conditions, immune effector functions are eliminated or reduced. Where the therapeutic is used in the treatment of a tumor or cancer, immune effector functions can be enhanced to improve the anti-tumor immune response and therapeutic efficacy. Additionally, or alternatively, the Fc region is modified to enhance FcRn recycling, to increase the in vivo serum stability and half-life of the molecules provided herein.

For purposes here, the Fc regions or domains are modified, particularly to decrease or eliminate ADCC. Small molecule therapeutics, such as antibody fragments (e.g., Fabs, scFvs, dAbs), are advantageous. They can be produced in high yields, and have other advantageous properties. They exhibit enhanced tissue penetration and target accessibility compared to monoclonal antibodies (mAbs), and they can prevent undesirable effects of mAbs, such as, for example, receptor clustering, the activation of immune effector functions, poor tissue penetration and lack of access to targets in poorly vascularized areas. Small antibody fragments, however, have poor pharmacokinetic properties. For example, due to their small size, dAbs and other antibody fragments are rapidly cleared by the kidneys, as molecules that are 50-60 kDa in size or smaller are subject to renal filtration. The rapid clearance and short elimination half-life of small antibody fragments, which can be less than a few hours, decreases the in vivo efficacy and necessitates frequent administration and/or continuous infusion.

Several methods can be used to increase the retention and in vivo half-life of small antibody fragments, such as dAbs. For example, as provided herein, the dAb(s) in the TNFR1 antagonist, TNFR2 agonist and combination/multi-specific constructs is/are fused to a linker that is or includes a half-life extender, such as, for example, the Fc region of an IgG, such as IgG1 or IgG4. The Fc can be a monomer or a dimer.

Fusion of small antibody fragments, such as dAbs, to the Fc region of an IgG molecule increases the size of the molecule, thereby protecting it from being cleared/excreted from the body, and mediates binding to the neonatal Fc receptor (FcRn) expressed on endothelial cells, which protects antibodies from lysosomal degradation and prolongs the in vivo half-life of the antibody. The addition of an Fc, however, can introduce unwanted properties, such as the induction of immune effector functions that can result in complement activation, the release of proinflammatory cytokines and cytotoxicity. Because TNFR1 is almost universally expressed, and TNFR2 is expressed by many tissues, it generally is not desirable to use ADCC-enhanced antibodies, but rather rely on the antagonist activity of the antibody for efficacy.

As described herein, the Fc region in the TNFR1 antagonist is modified to improve pharmacokinetic and pharmacodynamic (i.e., pharmacological) properties, and to eliminate undesirable properties. For example, the Fc region is modified to take advantage of/enhance neonatal FcR recycling to increase the in vivo half-life, and/or is mutated to eliminate Fc-related immune effector functions, such as antibody-dependent cellular cytotoxicity (ADCC; also known as antibody-dependent cell-mediated cytotoxicity), antibody-dependent cell-mediated phagocytosis (ADCP), and complement-dependent cytotoxicity (CDC). Additionally, in embodiments where the construct is multi-specific, such as bispecific, such as embodiments in which it contains an TNFR1 antagonist and an TNFR2 agonist, and contains an Fc dimer, the dimer is mutated to introduce knobs-in-holes to prevent homodimerization. Numerous modifications to Fc portions (or regions) are known to those of skill in the art (see, e.g., Li et al., (2014) Expert Opin Ther Targets 18:335-350).

i. Modifications to the Fc Portions

### a) Knobs-in-Holes

Bispecific antibodies (bsAbs) include two distinct antigen-binding sites, allowing for an alternative therapeutic approach to conventional therapeutic monoclonal antibodies (mAbs), whereby, limitations associated with mAbs, such as receptor co-clustering, can be avoided. While small antibody fragments are easier and less expensive to produce in high yields, and can easily penetrate tissues, they are associated with limitations, such as poor stability, solubility, and pharmacokinetic properties. For example, their small size results in shorter serum half-lives, reduced tissue retention and rapid clearance from the blood through the kidneys. As a result, IgG-like bi-specific (bs) Abs, which do not have the same limitations, are advantageous. For example, bsAbs can include an Fc region to increase the serum half-life, and also, to permit effector functions where desirable. The production of high yields of purified bsAbs, however, can be challenging, as homodimerization of the heavy chains must be prevented. The “knobs-in-holes” (KiH; also known as “knobs-into-holes”) approach provides a solution to this problem. The C_{H}3 domains of antibody (IgG) heavy chains are engineered for heterodimerization, to allow for the construction of Fc-containing bi-functional therapeutic molecules that will not self-associate.

The knobs-in-holes approach involves asymmetrically mutating interfacial residues in the CH3 domains of the two parental heavy chains in a complementary manner. “Knobs” are created by replacing amino acids with small side chains with amino acids with larger side chains, such as tyrosine or tryptophan, at the interface between CH3 domains, and “holes” are created by replacing amino acids with large side chains with amino acids with smaller ones, such as alanine or threonine. The knob and hole variants heterodimerize by virtue of the knob inserting into a correspondingly designed hole on the partner C_{H}3 domain. Knob-knob association is prevented due to steric repulsion, and hole-hole homodimers are destabilized. The knob mutation, for example, can be S354C, T366Y, T366W, or T394W, and the hole mutation can be Y349C, T366S, L368A, F405A, Y407T, Y407A, or Y407V (all by EU numbering). It has been shown that knobs created towards the center of the dimer interface, such as at residue T366, are more disruptive to homodimer formation than those located near the edge of the dimer interface. Residue T366 on the first CH3 domain is within hydrogen-bonding distance of residue Y407 on the second or partner C_{H}3 domain, thus, T366Y and Y407T represent a common knob-in-hole pair; this pair has been shown to generate heterodimers in yields of over 90% (see, e.g., Ridgway et al. (1996) Protein Eng. 9(7):617-621).

The IgG Fc regions, for example, in the bispecific TNFR1 antagonist/TNFR2 agonist constructs provided herein can be modified using the knobs-in-holes approach to generate heterodimerized molecules in high yields. Table 6, below, shows the corresponding knob and hole mutations by Kabat numbering and sequential numbering, with reference to the sequence of the IgG1 heavy chain constant domain set forth in SEQ ID NO:9. Any mutations known to those of skill in the art that introduce knobs-in-holes can be employed in constructs herein.

TABLE 6
IgG1 Fc Modifications that Introduce Knobs-into-Holes

| | | | Modifications by |
|---|---|---|---|
| | Modifications | Modifications | Sequential |
| Modification | by EU | by Kabat | Numbering |
| Type | Numbering | Numbering | (SEQ ID NO: 9) |
| Knob | S354C | S375C | S237C |
| Knob | T366Y | T389Y | T249Y |
| Knob | T366W | T389W | T249W |
| Knob | T394W | T422W | T277W |
| Hole | Y349C | Y370C | Y232C |
| Hole | T366S | T389S | T249S |
| Hole | L368A | L391A | L251A |
| Hole | F405A | F436A | F288A |
| Hole | Y407T | Y438T | Y290T |
| Hole | Y407A | Y438A | Y290A |
| Hole | Y407V | Y438V | Y290V |

b) Modifications that Enhance Neonatal Fc

### Receptor (FcRn) Recycling

There are numerous approaches to increasing the short serum half-life of small polypeptide or protein therapeutics. PEGylation, which is increases the serum half-life of small protein therapeutics, has a disadvantage. PEGylation can decrease potency or activity of a protein therapeutic, can result in heterogeneity, and can result immunoreactivity of the protein. Other approaches involve fusion to albumin, which can improve protein circulation by increasing the molecular weight and reducing renal clearance.

Serum half-life also can be increased by fusion to Fc portion of IgGs. The long circulating half-life of approximately 2-3 weeks, and slow clearance rate, of IgGs results at least in part, from their interaction with the neonatal Fc receptor (FcRn), which binds IgGs with high affinity at acidic pH, and releases them at neutral or higher pH. FcRn binds to the Fc portion (within the C_{H}2-C_{H}3 domains) of pinocytosed IgGs in the acidic (~pH 6) endosome in a 2:1 FcRn:IgG configuration (bivalent interaction), traffics them away from the lysosomal degradation pathway and to the cell surface, and recycles them back into circulation after exposure to the extracellular physiological pH (~7.4), at which the Fc-FcRn complex dissociates. Poor binding to FcRn at acidic pH results in trafficking of an antibody to the lysosome where it is degraded. Recycling receptors, such as FcRn, also provide a route for the transport of IgGs across the epithelium (transcytosis) and into the blood stream. Leveraging the interaction with FcRn can improve protein transport across epithelial barriers, such as in the gut and the lungs, allowing for noninvasive administration. Residues in the Fc CH2 and CH3 domains are involved in FcRn binding, and their mutation in mAbs has been shown to affect the in vivo serum half-life. The circulation and delivery of small protein therapeutics can be improved by fusing them to the Fc domain of IgG, such that the resulting fusion proteins bind to FcRn and take advantage of the IgG serum stabilization pathway. Fusion with an Fc domain also increases the molecular weight of the therapeutic, reducing renal clearance, but can be undesirable due to the potentially reduced tissue penetration and specific activity of the fusion protein. Alternatively, studies have shown that short FcRn-binding peptides (FcRnBPs) allow for the interaction of small proteins with FcRn, obviating the need for fusion to a high molecular weight Fc domain. For example, fusion with an FcRnBP increases the molecular weight by approximately 3 kDa, in comparison to fusions with Fc or albumin, which increase the molecular weight by approximately 50-70 kDa (see, e.g., Datta-Mannan et al. (2019) Biotechnol. J. 14:1800007; Sockolosky et al. (2012) Proc. Natl. Acad. Sci. USA 109(40):16095-16100).

For example, short (16 residue) linear and cyclic FcRnBPs (see, e.g., SEQ ID NOs: 48-51) have been fused to the C-terminus, N-terminus, or both, of Fab heavy and light chains (FcRnBP-Fab constructs), with 1-4 FcRnBPs per Fab. Studies of the pharmacokinetics in cynomolgus monkeys have shown that the FcRn binding of FcRnBP-Fab constructs increases as the number of peptides fused to the Fab increases. This results from increased avidity, with constructs containing four linear FcRnBPs fused to the N- and C-termini of the heavy and light chains of the Fab showing the greatest improvement in pharmacokinetics in cynomolgus monkeys relative to the parental Fab. For example, the half-life improved from 3.7 hours for the parental Fab, to between 15-60 hours for the various FcRnBP-Fab constructs (see, e.g., Datta-Mannan et al. (2019) Biotechnol. J. 14:1800007). While these results indicate an improvement in serum half-life, it is still much lower than the half-life for an IgG, which is about 2-3 weeks. The use of FcRnBPs also does not reduce renal clearance, as they do not significantly increase the molecular weight of the therapeutics.

As discussed above, fusion with an IgG Fe increases the half-life of small protein therapeutics by taking advantage of FcRn binding, and also by increasing the molecular weight of the therapeutic, such that it is less rapidly cleared from the body, for example, by the kidneys. To improve the pharmacokinetics and overall pharmacology, residues within the Fc region can be mutated to increase the affinity for FcRn, generally by greater than 30-fold, further increasing the in vivo half-life. The Fc region spanning the interface of the CH2 and C_{H}3 domains interacts with FcRn. Human Fc residues identified to play a role in FcRn binding include, for example, L251, M252, 1253, S254, L309, H310, Q311, L314, E380, N434, H435 and Y436 (by EU numbering, see Table 1). Mutations in residues located at the Fc-FcRn interface, including M252, S254, T256, H433, N434 and Y436 (by EU numbering), improve the stability of the human FcRn-IgG1 complex. For example, the replacements M252Y/S254T/T256E and H433K/N434F/Y436H result in an 11-fold and 6.5-fold improvement in binding to human FcRn at pH 6.0 relative to the wild-type IgG1, respectively, with efficient release at pH 7.4. The combination of these replacements results in a 57-fold increase in binding affinity to FcRn. Additional mutations in IgG1 Fc that showed an improvement in binding to FcRn include, for example, M252W, M252Y, M252Y/T256Q, M252F/T256D, E380A, and N434F/Y436H (see, e.g., Dall'Acqua et al. (2002) J. Immunol. 169:5171-5180).

The triple substitution M252Y/S254T/T256E, when introduced into the CH2 domain of MEDI-524, a humanized anti-respiratory syncytial virus (RSV) mAb, increased the serum half-life of the mAb approximately 4-fold in cynomolgus monkeys when compared to unmodified MEDI-524. When introduced into the Fc portion of MEDI-522, a humanized, affinity-optimized mAb directed against the human aᵥθ₃ integrin complex, the replacements M252Y/S254T/T256E (YTE) reduced its ADCC activity and its binding to human FcγRIIIA (F158 allotype). The ADCC activity of MEDI-522-YTE can be restored, and increased in comparison to unmodified MEDI-522, by introduction of the ADCC-enhancing replacements S239D/A330L/I332E (by EU numbering), indicating that the replacements YTE provide a reversible mechanism to modulate the ADCC function of a human IgG1 (see, e.g., Dall'Acqua et al. (2006) J. Biol. Chem. 281(33):23514-23524).

Residues at positions 250, 314 and 428 (by EU numbering) of the human IgG heavy chain, which are conserved among all four human IgG subtypes, also are located near the Fc-FcRn interface. The mutations T250Q, M428L and T250Q/M428L, when introduced into the Fe of a human IgG2 mAb, resulted in an increase in binding to FcRn at pH 6.0 of ~3-, 7- and 28-fold, respectively, with no binding observed at pH 7.5. When the pharmacokinetics of the mutants were evaluated in rhesus monkeys, it was found that the mean clearance, i.e., the volume of serum antibody cleared per unit of time, was ~-1.8-fold lower for the M428L mutant, and ~2.8-fold lower for the T250Q/M428L mutant, while the elimination half-life was ~1.8-fold longer for the M428L mutant and -1.9-fold longer for the T250Q/M428L mutant, compared to unmodified antibody. Since these residues are conserved among IgG subtypes, the mutations M428L and T250Q/M428L are expected to have similar effects in human IgG1, IgG3 and IgG4 antibodies (see, e.g., Hinton et al. (2004) J. Biol. Chem. 279(8):6213-6216). The modifications T250R/M428L were shown to result in selective binding to FcRn at pH 6.0, and a 2.8-fold decreased degradation of serum IgG2 and IgG1 in rhesus monkeys (see, e.g., Saxena et al. (2016) Front. Immunol. 7:580).

The mutation N434A (by EU numbering), when introduced into the human anti-HER2 IgG1 trastuzumab, resulted in ~4-fold higher affinity towards human FcRn over unmodified antibody at pH 6, but negligible binding at pH 7.4. The N434A variant had increased exposure, decreased clearance (~2-fold) and increased half-life (~2-fold) compared to the wild-type antibody when tested in vivo in cynomolgus monkeys. In contrast, the mutation N434W, which resulted in ~80-fold increased binding to FcRn at pH 6, exhibited a clearance rate similar to wild-type; this mutant also exhibited significant binding to FcRn at pH 7.4, indicating that maintaining pH-dependent binding of Fc mutants to FcRn is critical for improving the in vivo pharmacokinetics (Yeung et al. (2009) J. Immunol. 182:7663-7671). The N434A mutation also counters the poor FcRn affinity that can result from the introduction of mutations that increase binding to FcγRs; N434A is typically added to the mutations S298A/E333A/K333A to create a variant with enhanced FcγR binding and normal or improved FcRn binding. Fc mutations that improve FcRn binding also include N434Y, E294del/T307P/N434Y and T256N/A378V/S383N/N434Y. The E294 deletion results in higher sialylation of the N297 glycan on the Fc, which increases antibody half-life in vivo. Indicating that sialylation also plays a role in regulating serum half-life (see, e.g., Saunders, K. O. (2019) Front. Immunol. 10:1296).

The replacements M428L/N434S (by EU numbering), when introduced into the humanized anti-VEGF IgG1 antibody bevacizumab (Avastin®), resulted in an 11-fold increase in affinity to FcRn at pH 6.0, and extended the in vivo serum half-life in cynomolgus monkeys from 9.7 days to 31.1 days, representing a 3.2-fold improvement. The M428L/N434S modification resulted in similar increases in FcRn binding and half-life extension when introduced into the anti-EGFR antibody cetuximab, which is rapidly cleared due to receptor-mediated internalization. The half-life extension of these anti-tumor antibodies correlated with enhanced tumor reduction in vivo in a mouse model, indicating that the in vivo therapeutic efficacy of the antibodies is increased when the pharmacokinetics, such as clearance rate, are improved. Other mutations engineered into the bevacizumab Fc include (by EU numbering): N434S, with ~3-fold improvement in FcRn binding and ~2.8 fold increase in serum half-life in mice; V259I/V308F, with ~6-fold improvement in FcRn binding, and ~3-fold and ~2-fold increases in serum half-life in mice and cynomolgus monkeys, respectively; M252Y/S254T/T256E, with ~7-fold improvement in FcRn binding, and ~4-fold and 2.5-fold increases in serum half-life in mice and cynomolgus monkeys, respectively; and V259I/V308F/M428L, with ~20-fold improvement in FcRn binding, and ~4-5-fold and 2.6-fold increases in serum half-life in mice and cynomolgus monkeys, respectively (Zalevsky et al. (2010) Nat. Biotechnol. 28(2):157-159).

The above-identified mutations, and other such mutations, can be introduced into the IgG Fc region in constructs provided herein. These include constructs, such as those of Formulae 1 and 2, in which the linker includes an Fc or an Fc dimer, depending upon the structure of the construct.

In some embodiments, the IgG Fc regions in constructs herein, such bispecific TNFR1 antagonist/TNFR2 agonist constructs, and the TNFR1 antagonist constructs provided herein are modified to enhance neonatal FcR recycling to increase in vivo half-life. This can be effected by mutating residues at the interface of the CH2 and CH3 domains of IgG Fc, which are responsible for binding to FcRn. These include, but are not limited to, the residues T250, L251, M252, 1253, S254, T256, V259, T307, V308, L309, H310, L314, Q311, A378, E380, S383, M428, H433, N434, H435 and Y436, by EU numbering. Exemplary Fc modifications that increase binding to FcRn include, but are not limited to, one or more of T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259F1V308F, V259I/V308F/M428L, E294del/T307P/N434Y, T256N/A378V/S383N/N434Y, and combinations thereof, by EU numbering. Table 7, below, shows the corresponding mutations by Kabat numbering and sequential numbering, with reference to the sequence ofthe IgG1 heavy chain constant domain set forth in SEQ ID NO:9. Other modifications, known in the art to confer enhanced or increased FcRn binding also are contemplated for use herein.

TABLE 7
IgG1 Fc Modifications that Enhance FcRn Binding

| | Modifications | Modifications by |
|---|---|---|
| Modifications by EU | by Kabat | Sequential Numbering |
| Numbering | Numbering | (SEQ ID NO: 9) |
| T250Q | T263Q | T133Q |
| T250R | T263R | T133R |
| M252F | M265F | M135F |
| M252W | M265W | M135W |
| M252Y | M265Y | M135Y |
| S254T | S267T | S137T |
| T256D | T269D | T139D |
| T256E | T269E | T139E |
| T256Q | T269Q | T139Q |
| V259I | V272I | V142I |
| V308F | V327F | V191F |
| E380A | E405A | E263A |
| M428L | M459L | M311L |
| H433K | H464K | H316K |
| N434F | N465F | N317F |
| N434A | N465A | N317A |
| N434W | N465W | N317W |
| N434S | N465S | N317S |
| N434Y | N465Y | N317Y |
| Y436H | Y467H | Y319H |
| M252Y/T256Q | M265Y/T269Q | M135Y/ T139Q |
| M252F/T256D | M265F/T269D | M135F/T139D |
| M252Y/S254T/ | M265Y/S267T/ | M135Y/S137T/ |
| T256E | T269E | T139E |
| H433K/N434F/ | H464K/N465F/ | H316K/N317F/ |
| Y436H | Y467H | Y319H |
| N434F/Y436H | N465F/Y467H | N317F/Y319H |
| T250Q/M428L | T263Q/M459L | T133Q/M311L |
| T250R/M428L | T263R/M459L | T133R/M311L |
| M428L/N434S | M459L/N465S | M311L/N317S |
| V259I/V308F | V272I/V327F | V142I/ V191F |
| V259I/V308F/M428L | V272I/V327F/M459L | V142I/V191F/M311L |
| E294del/T307P/ | E311del/T326P/ | E177del/T190P/ |
| N434Y | N465Y | N317Y |
| T256N/A378V/ | T269N/A401V/ | T139N/A261V/ |
| S383N/N434Y | S408N/N465Y | S266N/N317Y |

### c) Enhancement of or Reduction/Elimination of Fc Immune Effector Functions

There are four human IgG subclasses that differ in effector functions, circulating half-life and stability. IgG1 has Fc effector functions, is the most abundant IgG subclass, and is the most commonly used subclass in FDA-approved therapeutic proteins. IgG2 is deficient in Fc effector functions, but dimerizes with other IgG2 molecules, and is unstable due to scrambling of disulfide bonds in the hinge region. IgG3 has Fc effector functions, and a very long, rigid hinge region. IgG4 is deficient in Fc effector functions, has a shorter circulating half-life than the other subclasses, and the IgG4 dimer is biochemically unstable due to the presence of a single disulfide bond in the hinge region, which leads to the exchange of H chains between different IgG4 molecules. Thus, Fc regions from IgG2 and IgG4 do not possess effector functions, and can be used in instances where effector functions are not required or would be detrimental, for example, in the context of autoimmune and inflammatory diseases and disorders.

Most approved therapeutic mAbs belong to the human IgG1 subclass, and can interact with the humoral and cellular components of the immune system. For example, antibodies engage the humoral immune response via interaction with complement protein C1q, which initiates the complement cascade, resulting in the formation of the membrane attack complex which induces cytolysis in the target cell (i.e., complement-dependent cytotoxicity (CDC)), and engage the cellular immune response by interaction with Fc gamma receptors (FcγRs). The FcγRs include the FcγRI (CD64), FcγRII (CD32) and FcγRIII (CD16) classes that differ in their cell surface expression and Fc binding affinities. The five activating FcγRs include the high affinity FcγRI that can bind monovalent antibodies, and the lower affinity FcγRIIa, FcγRIIc, FcγRIIIa and FcγRIIIb, which require avidity-based interactions. FcγRIIb is the only inhibitory receptor. Upon binding of an Fc to an activating receptor, intracellular signaling pathways, modulated through the phosphorylation of immunoreceptor tyrosine-based activation motifs (ITAMs), result in effector functions, such as antibody-dependent cell-mediated cytotoxicity (ADCC; also called antibody-dependent cellular cytotoxicity) and antibody-dependent cell-mediated phagocytosis (ADCP; also called antibody-dependent cellular phagocytosis), as well as inflammation due to the induction of cytokine secretion. Signaling via the inhibitory FcγRIIb, which is modulated through the phosphorylation of immunoreceptor tyrosine-based inhibitory motifs (ITIMs), recruits phosphatases that counter-balance the activating signaling pathways (see, e.g., Wang et al. (2018) Protein Cell 9(1):63-73).

The hinge and proximal CH2 amino acid sequence (lower hinge-upper CH2 domain region), and glycosylation of the conserved N297 residue (by EU numbering) in the CH2 domain Asn-X-Ser/Thr glycosylation motif of the Fc region, mediate the interactions of antibodies with FcγRs and complement protein C1q. Antibody/Fc engineering has been used to modify the immune effector functions of antibodies by altering their binding to C1q and various Fcγ receptors. The CDC, ADCC and ADCP activities of therapeutic mAbs can thus be increased or decreased, depending on the application. For example, the efficacy of anti-cancer mAbs depends in part on their induction of FcγR effector functions. The effector function includes the activation of natural killer (NK) cells via FcγRIIIa and the subsequent ADCC activity and release of inflammatory cytokines, the induction of macrophage-mediated ADCP via interactions with multiple FcγRs, and the recruitment and activation of other immune cells, such as neutrophils, the primary receptor for NK cell-mediated ADCC. FcγRIIIa has two polymorphic variants: one with V158, which has a higher affinity for IgG1; and one with F158, with a lower affinity for IgG1. Cancer patients with the high affinity V158 polymorphism can have better outcomes following treatment with cetuximab, trastuzumab and rituximab, compared to patients with the low affinity F158 polymorphism. Results such as these highlight the role that FcγR-mediated immune effector functions play in therapies, and indicate that engineering antibodies and related molecules to have increased affinity to FcγRs can enhance the therapeutic efficacy (see, e.g., Wang et al. (2018) Protein Cell 9(1):63-73).

Residues in the lower hinge and proximal CH2 regions of IgGs have been determined to be critical for binding to FcγRs. Residues that are within 5 angstroms from the FcγR:Fc interface for FcγRI, FcγRIIa, FcγRIIb and FcγRIIIb, include the residues (by EU numbering) P232, E233, L234, L235, G236, G237, P238, S239 (corresponding to residues P115-S122, with reference to SEQ ID NO:9), D265, V266, S267, H268, E269, D270 (corresponding to residues D148-D153, with reference to SEQ ID NO:9), Y296, N297, S298, T299 (corresponding to residues Y179-T182, with reference to SEQ ID NO:9), and N325, K326, A327, L328, P329, A330, P331 and 1332 (corresponding to residues N208-I215, with reference to SEQ ID NO:9) (see, e.g., Wang et al. (2018) Protein Cell 9(1):63-73).

Modifications of Fe that enhance or decrease ADCC activity and/or enhance affinity/binding to receptors are known to those of skill in the art. For example, Fc modifications that increase the IgG1 affinity for and binding to FcγRIIIa, and/or enhance ADCC function, include the replacements (by EU numbering): F243L/R292P/Y300L/V305I/P396L, L235V/F243L/R292P/Y300L/P396L, F243L/R292P/Y300L, S239D, I332E, S239D/I332E, S239D/A330L/I332E, S298A/E333A/K334A, and the combinations of L234Y/L235Q/G236W/S239M/H268D/D270E/S298A in one heavy chain and D270E/K326D/A330M/K334E in the opposing heavy chain, and L234Y/G236W/S298A in one heavy chain and S239D/A330L/I332E in the opposing heavy chain. Additionally, the mutations A327Q/P329A (interact with FcγRI), D265A/S267A/H268A/D270A/K326A/S337A (interact with FcγRIIa), G236A (interacts with FcγRIIa), and T256A/K290A/S298A/E333A/K334A (interact with FcγRIIIa), result in high affinity interactions with FcγRs.

Fc modifications that increase binding to FcγRIIa and FcγRIIIa, and enhance ADCC and ADCP, include (by EU numbering) G236A/I332E, G236A/S239D/I332E (also increases binding to FcγRI), and G236A/S239D/A330L/I332E (see, e.g., Wang et al. (2018) Protein Cell 9(1):63-73; Saxena et al (2016) Front. Immunol. 7:580; and Saunders, K. O. (2019) Front. Immunol. 10:1296).

Glyco-engineering of IgGs, which contain a conserved N-linked glycosylation site at residue N297 in the CH2 domain, can enhance Fc effector function. Glycosylation of N297 is essential for maintaining Fc conformation and mediating its interactions with FcγRs (and C1q). The glycan present at residue N297 typically has two N-acetylglucosamine (GlcNAc), three mannose, and two more GlcNAc linked to the mannose, to form a biantennary complex glycan. Additional fucose, galactose, sialic acid and GlcNAc can be added to the core glycan structure. IgGs found circulating in human sera generally are fucosylated, but recombinant IgG production can alter the glycan composition by expressing the antibody in plant cells, knocking in or out specific glycosidases, or in vitro enzymatic digestion of the glycosylated IgG; because both heavy chains are glycosylated, a single IgG molecule can have glycan heterogeneity. The glycan directly affects FcγR binding. For example, the N297 glycan on the Fc can clash with glycans on the FcγRIII protein, resulting in poor engagement of effector cells that mediate ADCC. Fc regions containing different glycans at N297 adopt different hinge region conformations, which can affect the Fc's ability to interact with FcγRs. Expression of β(1,4)-N-acetylglucosaminyltransferase III when expressing IgG generates an antibody that is glycosylated at position N297 with a biantennary glycan; this antibody has increased binding to FcγRIIIa and enhanced ADCC activity. It has been demonstrated that fucose deficient (afucosylated/non-fucosylated) IgG1s exhibit up to 50-fold increased binding to FcγRIIIa and enhanced ADCC activity. Two glyco-engineered (afucosylated) mAbs, obinutuzumab (anti-CD20) and mogamulizumab (anti-CCR4) have been approved for clinical use, indicating the potential for glyco-engineering for enhanced effector function, and its translation into clinically approved therapeutics (see, e.g., Wang et al. (2018) Protein Cell 9(1):63-73; Saxena et al. (2016) Front. Immunol. 7:580; and Saunders, K. O. (2019) Front. Immunol. 10:1296).

The Fc also can be modified to bind with a wider range of Fc receptors. Fc receptors for isotypes other than gamma (i.e., IgA, IgM and IgE) exist on certain leukocytes, and by modifying an Fc region to engage with multiple Fc receptors, an antibody with expanded abilities to engage effector cells is created. Neutrophils, which are the most abundant leukocytes in the body, engage the Fc of IgA antibodies via the FcαRI receptor. For example, to engage FcγRs and FcαRI, single domains of IgA2 were added to the end of the IgG1 constant region, creating a four domain constant region, CH1g-CH2g-CH3g-CH3a. The C_{H}1 domain of IgG1 was replaced with the alpha 1 constant region domain, generating a constant region (CH1a-CH2g-CH3g-CH3a) that is closer in structure to the alpha constant region. These four-domain, cross-isotype IgGA chimeric antibodies bound to J chain similarly to natural IgA2, had reduced transport by polymeric Ig receptor, had a 3-5-fold decrease in FcγRI affinity, and the short serum half-life of IgA2 instead of the protracted serum circulation of IgG1. The four-domain, cross-isotype IgGA chimeric antibodies, however, had the ability to mediate complement-dependent lysis of sheep red blood cells and were more pH-resistant than IgG1. Another cross-isotype Fc was created by fusing the gamma 1 and alpha constant regions together to create a tandem G1-A Fc region, in which the hinge, C_{H}2 and C_{H}3 domains of IgA2 were fused to the C-terminus of IgG1. This tandem cross-isotype IgG/IgA fusion showed similar expression levels, antigen binding and thermostability as IgG1, and, in vitro, bound to FcαRI and FcγRI, FcγRII, FcγRIIIa and FcRn, with affinities similar to wild-type IgA and IgG, respectively. The binding to various FcRs resulted in ADCC activity with polymorphonuclear cells and NK cells; C1q binding, however, was reduced 3-fold compared to IgG1. The tandem IgG/IgA had an in vivo half-life similar to that of IgG1 in BALB/c mice. An alternative cross-isotype antibody was created by replacing the CH3 domain and C_{H}2 α1 loop residues 245-258 (by EU numbering, corresponding to the sequence PKPKDTLMISRTPE; (residues 128-141 of SEQ ID NO:9)) of the IgG1 constant region, with the structurally analogous regions of the IgA constant region. This chimeric Fc was able to bind FcγRI, FcγRIIa and FcαRI, and antibodies containing the chimeric Fc mediated ADCC with polymorphonuclear cells and ADCP with macrophages, and activated complement, but lacked binding to FcRn, which regulates antibody half-life; thus, further optimization is required for effective in vivo use (see, e.g., Saunders, K. O. (2019) Front. Immunol. 10:1296).

Another approach to enhance FcγR binding is the multimerization of IgG, which has shown promise in the treatment of autoimmune diseases. The IgG multimers are generated, for example, by adding heterologous multimerization domains such as isoleucine zippers, or by adding another hinge region at the N-terminus of the natural hinge, or by adding another hinge region at the C-terminus of the CH3 domain. IgG hexamers are created by appending the IgM tailpiece to the C-terminus of the IgG1 Fc and creating a cysteine bond at position 309; this multimeric IgG bound strongly to FcγRI, FcγRIIa and FcγRIIIa, and weakly to FcγRIIb and FcγRIIIb. The various multimeric IgGs have increased binding to FcγRI, FcγRIIb and FcγRIII, compared to monomeric IgG, and have shown promise in preclinical models of arthritis, neuropathy, and autoimmune myasthenia gravis. This multimeric IgG design is being further optimized to fine-tune which immune receptors, including FcRn, can bind to the multimer (see, e.g., Saunders, K. O. (2019) Front. Immunol. 10:1296).

Residues in the Fc region of IgG that are involved in the interaction with and binding to C1q (and hence, CDC) include (by EU numbering) S267, D270, K322, K326, P329, P331 and E333. Fc modifications that have been shown to enhance CDC by increasing C1q binding include, for example, K326A, E333A, K326A/E333A, K326W, K326W/E333S, K326M/E333S, C220D/D221C, H268F/S324T, S267E, H268F, S324T, S267E/H268F/S324T, and G236A/I332E/S267E/H268F/S324T (all by EU numbering). In the upper hinge region of the IgG1 Fc, substituting Trp, in various combinations, at positions 222, 223 and 224 (i.e., K222W, T223W, and H224W, by EU numbering), increased C1q binding and CDC activity relative to wild-type IgG1 without affecting FcγRIIIa binding and ADCC activity. Specifically, the mutations included K222W/T223W, K222W/T223W/H224W and D221W/K222W. The mutations C220D/D221C and C220D/D221C/K222W/T223W, also increased C1q binding and CDC activity (see, e.g., Wang et al. (2018) Protein Cell 9(1):63-73; Saxen et al. (2016) Front. Immunol. 7:580; Saunders, K. O. (2019) Front. Immunol. 10:1296; and Dall'Acqua et al. (2006) J. Immunol. 177:1129-1138).

IgG3 has the best in vitro binding to C1q; combining the C_{H}1 and hinge regions of IgG1 with the CH2 and CH3 regions of IgG3 (to retain ADCC activity from IgG1 and CDC activity from IgG3), creating IgG1/IgG3 cross-subtype antibodies, also increases C1q binding and enhances CDC activity. Another IgG1/IgG3 cross-subtype antibody with increased C1q binding and enhanced CDC activity includes the C_{H}1, hinge and CH3 of IgG1, and the CH2 of IgG3; these modifications allow for increased Cql binding since C1q binds the CH2 domain, as well as easy purification, since protein A binds the CH3 domain. Additionally, the modifications E345R/E430G/S440Y, which result in the formation of IgG hexamers with K322 oriented in a position to favorably interact with the hexameric C1q headpiece, enhanced CDC activity. The mutation E345R alone also results in IgG hexamer formation, with increased C1q binding and enhanced CDC activity (see, e.g., Wang et al. (2018) Protein Cell 9(1):63-73; Saxena et al. (2016) Front. Immunol. 7:580; Saunders, K. O. (2019) Front. Immunol. 10:1296).

Glycoengineering also can be used to improve complement binding; the N297 glycan within the CH2 domain of the Fc can be modified to improve CDC activity. For example, an overabundance of galactosylation in the IgG1 Fc increases C1q binding and CDC activity compared to the unmodified glycoform of IgG1, and also improves thermostability. Thus, galactosylating the Fc can be used to generate a stable biologic with enhanced CDC activity (see, e.g., Saunders (2019) Front. Immunol. 10:1296).

Table 8, below, summarizes the Fc modifications that increase binding to FcγRs or C1q, and thus, enhance immune effector functions, including ADCC, ADCP and CDC, and provides the corresponding modifications by Kabat numbering and by sequential numbering, with reference to the sequence of the IgG1 heavy chain constant domain set forth in SEQ ID NO:9. Any one or more of these modifications, alone or in various combinations, can be introduced into the IgG1 Fc portions of the constructs provided herein. Other modifications, known in the art to confer enhanced or increased immune effector functions, also are contemplated for use herein.

TABLE 8
IgG1 Fc Modifications that Enhance Immune Effector Functions

| | | Modifications by | |
|---|---|---|---|
| Modifications by EU | Modifications by Kabat | Sequential Numbering | |
| Numbering | Numbering | (SEQ ID NO: 9) | Effects |
| S239D | S252D | S122D | Increase binding to |
| | | | FcγRIIIa; enhance |
| | | | ADCC |
| I332E | I351E | I215E | Increase binding to |
| | | | FcγRIIIa; enhance |
| | | | ADCC |
| S239D/1332E | S252D/I351E | S122D/I215E | Increase binding to |
| | | | FcγRIIIa; enhance |
| | | | ADCC |
| S239D/A330L/1332E | S252D/A349L/1351E | S122D/A213L/1215E | Increase binding to |
| | | | FcγRIIIa; enhance |
| | | | ADCC |
| S298A/E333A/K334A | S317A/E352A/K353A | S181A/E216A/K217A | Increase binding to |
| | | | FcγRIIIa; enhance |
| | | | ADCC |
| F243L/R292P/Y300L/V305M | F256L/R309P/Y319L/V324M | F126L/R175P/Y183L/V188M | Increase binding toM |
| I/P396L | I/P424L | I/P279L | FcγRIIIa and |
| | | | FcγRIIa; enhance |
| | | | ADCC |
| L235V/F243L/R292P/Y300 | L248V/F256L/R309P/Y319 | L118V/F126L/R175P/Y183 | Increase binding to |
| L/P396L | L/P424L | L/P279L | FcγRIIIa; enhance |
| | | | ADCC |
| F243L/R292P/Y300L | F256L/R309P/Y319L | F126L/R175P/Y183L | Increase binding to |
| | | | FcγRIIIa; enhance |
| | | | ADCC |
| L234Y/G236W/S298A (1^{st} | L247Y/G249W/S317A (1^{st} | L117Y/G119A/S181A (1^{st} | Increase binding to |
| heavy chain) and | heavy chain) and | heavy chain) and | FcγRIIIa; enhance |
| S239D/A330L/1332E (2^{nd} | S252D/A349L/1351E (2^{nd} | S122D/A213L/1215E (2^{nd} | ADCC |
| heavy chain) | heavy chain) | heavy chain) | |
| L234Y/L235Q/G236W/S23 | L247Y/L248Q/G249W/S25 | L117Y/L118Q/G119W/S12 | Increase binding to |
| 9M/H268D/D270E/S298A | 2M/H281D/D283E/S317A | 2M/H151D/D153E/S181A | FcγRIIIa; enhance |
| (1^{st} heavy chain) and | (1^{st} heavy chain) and | (1^{st} heavy chain) and | ADCC |
| D270E/K326D/A330M/K3 | D283E/K345D/A349M/K3 | D153E/K209D/A213M/K2 | |
| 34E (2^{nd} heavy chain) | 53E (2^{nd} heavy chain) | 17E (2^{nd} heavy chain) | |
| A327Q/P329A | A346Q/P348A | A210Q/P212A | Increase binding to |
| | | | FcγRI |
| D265A/S267A/H268A/D27 | D278A/S280A/H281A/D28 | D148A/S150A/H151A/D15 | Increase binding to |
| 0A/K326A/S337A | 3A/K345A/S357A | 3A/K345A/S220A | FcγRIIa |
| T256A/K290A/S298A/E33 | T269A/K307A/S317A/E35 | T139A/K173A/S181A/E21 | Increase binding to |
| 3A/K334A | 2A/K353A | 6A/K217A | FcγRIIIa |
| G236A | G249A | G119A | Increase binding to |
| | | | FcγRIIa; enhances |
| | | | ADCP |
| G236A/1332E | G249A/1351E | G119A/1215E | Increase binding to |
| | | | FcγRIIa and |
| | | | FcγRIIIa; enhance |
| | | | ADCC and ADCP |
| G236A/S239D/1332E | G249A/S252D/I351E | G119A/S122D/1215E | Increase binding to |
| | | | FcγRI, FcγRIIa |
| | | | and FcγRIIIa; |
| | | | enhance ADCC |
| | | | and ADCP |
| G236A/S239D/A330L/1332 | G249A/S252D/A349L/1351 | G119A/S122D/A213L/1215 | Increase binding to |
| E | F | E | FcγRIIa and |
| | | | FcγRIIIa; enhance |
| | | | ADCC and ADCP |
| Biantennary glycan at N297 | Biantennary glycan at N314 | Biantennary glycan at N180 | Increases binding |
| | | | to FcγRIIIa; |
| | | | enhances ADCC |
| Afucosylated glycan at | Afucosylated glycan at | Afucosylated glycan at | Increases binding |
| N297 | N314 | N180 | to FcγRIIIa; |
| | | | enhances ADCC |
| K326W | K345W | K209W | Increase binding to |
| | | | C1q; enhance CDC |
| K326A | K345A | K209A | Increase binding to |
| | | | C1q; enhance CDC |
| E333A | E352A | E216A | Increase binding to |
| | | | C1q; enhance CDC |
| K326A/E333A | K345A/E352A | K209A/E216A | Increase binding to |
| | | | C1q; enhance CDC |
| | | | and preserve |
| | | | ADCC activity |
| K326W/E333S | K345W/E352S | K209W/E216S | Increase binding to |
| | | | C1q; enhance CDC |
| K326M/E333S | K345M/E352S | K209M/E216S | Increase binding to |
| | | | C1q; enhance CDC |
| | | | and preserve |
| | | | ADCC activity |
| K222W/T223W | K235W/T236W | K105W/T106W | Increase binding to |
| | | | C1q; enhance CDC |
| K222W/T223W/H224W | K235W/T236W/H237W | K105W/T106W/H107W | Increase binding to |
| | | | C1q; enhance CDC |
| D221W/K222W | D234W/K235W | D104W/K105K | Increase binding to |
| | | | C1q; enhance CDC |
| C220D/D221C | C233D/D234C | C103D/D104C | Increase binding to |
| | | | C1q; enhance CDC |
| | | | and preserve |
| | | | ADCC activity |
| C220D/D221C/K222W/T2 | C233D/D234C/K235W/T2 | C103D/D104C/K105W/T1 | Increase binding to |
| 23W | 36W | 06W | C1q; enhance CDC |
| H268F/S324T | H281F/S343T | H151F/S207T | Increase binding to |
| | | | C1q; enhance CDC |
| S267E | S280E | S150E | Increase binding to |
| | | | C1q; enhance CDC |
| H268F | H281F | H151F | Increase binding to |
| | | | C1q; enhance CDC |
| S324T | S343T | S207T | Increase binding to |
| | | | C1q; enhance CDC |
| S267E/H268F/S324T | S280E/H281F/S343T | S150E/H151F/S207T | Increase binding to |
| | | | C1q; enhance CDC |
| G236A/I332E/S267E/H268 | G249A/I351E/S280E/H281 | G119A/I215E/S150E/H151 | Increase binding to |
| F/S324T | F/S343T | F/S207T | C1q; enhance CDC |
| E345R | E366R | E228R | Increase binding to |
| | | | C1q; enhance |
| | | | CDC; IgG1 |
| | | | hexamer formation |
| E345R/E430G/S440Y | E366R/E461G/S471Y | E228R/E313G/S323Y | Increase binding to |
| | | | C1q; enhance |
| | | | CDC; IgG1 |
| | | | hexamer formation |

Therapeutic antibodies also can be engineered to reduce or eliminate immune effector functions. For purposes herein, in some embodiments, it is of interest, for example, to reduce or eliminate ADCC activity. Constructs herein that include Fc generally are modified to reduce or eliminate ADCC activity.

It is of interest to reduce or eliminate immune effector functions, for example, where: the therapeutic antibodies are antagonistic in order to prevent receptor-ligand interactions and signaling; the antibodies are receptor agonists to crosslink receptors and induce signaling; the antibodies are drug delivery vehicles that deliver a drug to antigen-expressing target cells; and, where the reduction or elimination of effector functions prevents target cell death or unwanted cytokine secretion. Reduced effector function also prevents antibody-drug conjugates from interacting with FcγRs, which reduces off-target cytotoxicity. The importance of reducing or eliminating effector functions became evident following adverse events associated with the administration of the first approved mAb, muromonab, which was designed to prevent T cell activation in transplant patients receiving a donor kidney, lung or heart. Patients administered muromonab experienced a dangerous induction of pro-inflammatory cytokines (i.e., a cytokine storm); this was due, in part, to the interaction of muromonab with FcγRs (see, e.g., Wang et al. (2018) Protein Cell 9(1):63-73; and Saunders, K. O. (2019) Front. Immunol. 10:1296).

There are many known mutations that reduce or eliminate receptor function. For example, replacements L235E and F234A/L235A in human IgG4, and L235E and L234A/L235A (all by EU numbering) in human IgG1 reduce FcγR and C1q binding, and reduce effector functions, such as inflammatory cytokine release. Inflammatory cytokine release from therapeutic antibodies, can result in adverse effects. The replacements S228P/L235E, when introduced into IgG4, also reduce binding to FcγRs; the S228P mutation improves stability of IgG4. The mutations S228P/F234A/L235A in the IgG4 Fc decrease binding to FcγRI, IIa and IIIa, and reduce ADCC and CDC. The triple mutant L234E/L235F/P331S in IgG1 Fc decreases binding to FcγRI, FcγRII, FcγRIII and C1q, and reduces CDC, the mutations L234A/L235A/P329G in the IgG1 Fc eliminate FcγRI, FcγRII, FcγRIII and C1q binding, and reduce ADCP. The mutations L234F/L235E/P331S also reduce binding to FcγRs and C1q, and reduce effector functions of the IgG1 Fc. The mutations G237A and E318A in the IgG1 Fc each decrease binding to FcγRII and reduce ADCP; the mutations D265A and E233P decrease binding to FcγRI, FcγRII and FcγRIII, and decrease ADCC and ADCP, and the mutations G236R/L328R decrease binding to all FcγRs and reduce ADCC. Crystal structure data revealed that conformational changes at residue P329, which packs between two conserved tryptophan residues that occur in all FcγRs, form a “proline sandwich,” can be detrimental to the interaction with FcγRs, and that modifications at residue D270 can negatively impact interactions with C1q (see, e.g., Wang et al. (2018) Protein Cell 9(1):63-73; Saunders, K. O. (2019) Front. Immunol. 10:1296; International Application Publication No. WO 2019/226750).

Induction of the complement cascade is associated with antibody injection site adverse reactions, and eliminating C1q binding to Fc, which is the initial even in the activation of CDC. Modification of Fc region eliminate C1q binding can be used to eliminate CDC in constructs containing Fc regions. Many of the mutations that eliminate FcγR binding also eliminate C1q binding, as shown above. For example, the mutation A330L disrupts C1q binding and reduces CDC, and also eliminates FcγRIIb binding. The mutations D270A, P329A, K322A and P331A also result in reduced C1q binding and reduced CDC activity (see, e.g., Saunders, K. O. (2019) Front. Immunol. 10:1296).

Glyco-engineering can be used to ablate FcγR and C1q binding. As discussed elsewhere herein, the glycan at residue N297 is a complex biantennary glycan. Modification of this glycan to a high mannose glycan (i.e., high mannose glycosylation) reduces the affinity of IgG1 Fc for C1q and reduces CDC activity. Mutations in the Fc that reduce or eliminate C1q and FcγRI binding also can result in an increase in galactosylation and sialylation of the N297 glycan; such mutations include F241A, V264A and D265A, for example. The mutations N297A, N297Q, N297D and N297G, by EU numbering, remove the glycosylation site at N297 and reduce effector functions, such as CDC and ADCC, by abrogating Fc interactions with C1q and FcγRs, respectively. The combination N297G/D265A almost completely abrogates binding to FcγRs and C1q. An IgG3 Fc lacking glycosylation (the aglycone Fc) has reduced binding to FcγRI and C1q. (see, e.g., Wang et al. (2018) Protein Cell 9(1):63-73; Saunders, K. O. (2019) Front. Immunol. 10:1296).

To reduce or eliminate Fc effector functions, large portions of Fc regions from different subclasses, that lack opposing functions, can be exchanged to generate cross-subclass Fc regions. For example, IgG2 has poor FcγR binding but binds C1q, and IgG4 lacks C1q binding but reacts with FcγRs; thus, combinations of IgG2 and IgG4 CH domains that are devoid of both C1q and FcγR binding, can be constructed. In general, in IgG1/IgG4 chimeras, the hinge and C_{H}1 domain is from IgG2, and the CH2 and C_{H}3 domains are from IgG4. Since IgG1 and IgG3 recruit complement more effectively than IgG2 and IgG4, and because IgG2 and IgG4 are limited in their ability to induce ADCC, a cross-subclass approach can reduce effector function. For example, the anti-C5 mAb eculizumab, contains IgG2 residues 118-260 (by EU numbering; corresponding to residues 114-273 by Kabat numbering, and residues 1-139 with reference to SEQ ID NO:11), and IgG4 residues 261-447 (by EU numbering; corresponding to residues 274-478 by Kabat numbering, and residues 141-327 with reference to SEQ ID NO:15), and has limited or undetectable effector function. Similarly, an IgG2 variant (IgG2m4) with the point mutations H268Q/V309L/A330S/P331S from IgG4 (by EU numbering; corresponding to H281Q/V328L/A349S/P350S by Kabat numbering, and H147Q/V188L/A209S/P210S with reference to SEQ ID NO:11) lacks binding to all FcγRs and C1q and exhibits reduced effector functions. A variant (called IgG2a) containing the IgG2 to IgG4 cross-subclass mutations V309L/A330S/P331S (by EU numbering; corresponding to V328L/A349S/P350S by Kabat numbering, and V188L/A209S/P210S with reference to SEQ ID NO:11), and the non-germline mutations V234A/G237A/P238S/H268A (by EU numbering; corresponding to V247A/G250A/P251S/H281A by Kabat numbering, and V114A/G116A/P117S/H147A with reference to SEQ ID NO:11), eliminates binding to FcγRs and C1q and exhibit undetectable CDC, ADCC and ADCP activities. The IgG1/IgG4 cross-subclass variant IgG1a, which includes the mutations L234A/L235A/G237A/P238S/H268A/A330S/P331S, lacks binding to FcγRI and IIIa, and has very weak binding to FcγRIIa and IIb at high concentrations of antibody, resulting in reduced ADCC and CDC activities (see, e.g., Wang et al. (2018) Protein Cell 9(1):63-73; Saunders, K. O. (2019) Front. Immunol. 10:1296).

Tables 9 and 10, below, summarize some IgG1 and IgG4 Fc modifications that reduce or eliminate binding to FcγRs and/or C1q, and thus, reduce or eliminate immune effector functions, including ADCC, ADCP and CDC, which can be introduced into the Fc regions in constructs herein. The tables provide the corresponding modifications by Kabat numbering and by sequential numbering, with reference to the sequence of the IgG1 heavy chain constant domain set forth in SEQ ID NO:9, or the IgG4 heavy chain constant domain set forth in SEQ ID NO:15. Any one or more of these modifications, alone or in various combinations, can be introduced into the IgG1 Fc portions of the constructs provided herein. Other modifications, known in the art to reduce or eliminate immune effector functions, also are contemplated for use herein.

TABLE 9
IgG1 Fc Modifications that Reduce or Eliminate Immune Effector Functions

| | | Modifications by | |
|---|---|---|---|
| Modifications by | Modifications by | Sequential Numbering | |
| EU Numbering | Kabat Numbering | (SEQ ID NO: 9) | Effects |
| L235E | L248E | L118E | Reduces FcγR binding; reduces ADCC |
| L234A/L235A | L247A/L248A | L117A/L118A | Reduce FcγR and C1q binding; reduced |
| | | | ADCC, ADCP and CDC |
| L234E/L235F/P331S | L247E/L248F/P350S | L117E/L118F/P214S | Reduce FcγR and C1q binding; reduce |
| | | | CDC |
| L234F/L235E/P331S | L247F/L248E/P350S | L117F/L118E/P214S | Reduce FcγR and C1q binding; reduce |
| | | | effector functions |
| L234A/L235A/P329G | L247A/L248A/P348G | L117A/L118A/P212G | Eliminate FcγR and C1q binding; |
| | | | reduce ADCP and CDC |
| L234A/L235A/G237A/ | L247A/L248A/G250A/ | L117A/L118A/G120A/ | Reduced binding to FcγR1, IIa, IIb and |
| P238S/H268A/A330S/ | P251S/H281A/A349S/ | P121S/H151A/A213S/ | IIIa; reduced ADCC and CDC |
| P331S | P350S | P214S | |
| G236R/L328R | G249R/L347R | G119R/L211R | Reduced binding to FcγRs; reduced |
| | | | ADCC |
| G237A | G250A | G120A | Reduces binding to FcγRII; reduced |
| | | | ADCP |
| E318A | E337A | E201A | Reduces binding to FcγRII; reduced |
| | | | ADCP |
| D265A | D278A | D148A | Reduces binding to FcγRI, II, III; |
| | | | reduced ADCC and ADCP |
| E233P | E246P | E116P | Reduces binding to FcγRI, II, III; |
| | | | reduced ADCC and ADCP |
| N297A | N314A | N180A | Remove glycosylation site; decrease |
| | | | interaction with FcγRs; reduce effector |
| | | | functions (CDC, ADCC, ADCP) |
| N297Q | N314Q | N180Q | Remove glycosylation site; decrease |
| | | | interaction with FcγRs; reduce effector |
| | | | functions (CDC, ADCC, ADCP) |
| N297D | N314D | N180D | Remove glycosylation site; decrease |
| | | | interaction with FcγRs; reduce effector |
| | | | functions (CDC, ADCC, ADCP) |
| N297G | N314G | N180G | Remove glycosylation site; decrease |
| | | | interaction with FcγRs; reduce effector |
| | | | functions (CDC, ADCC, ADCP) |
| N297G/D265A | N314G/D278A | N180G/D148A | Reduces binding to FcγRs and C1q; |
| | | | reduces effector functions |
| A330L | A349L | A213L | Reduced C1q binding; reduced CDC |
| D270A | D283A | D153A | Reduced C1q binding; reduced CDC |
| P329A | P348A | P212A | Reduced C1q binding; reduced CDC |
| P331A | P350A | P214A | Reduced C1q binding; reduced CDC |
| K322A | K341A | K205A | Reduced C1q binding; reduced CDC |
| V264A | V277A | V147A | Reduced C1q binding; reduced CDC |
| F241A | F254A | F124A | Reduced C1q binding; reduced CDC |

TABLE 10
IgG4 Fc Modifications that Reduce or Eliminate Immune Effector Functions

| | | Modifications by | |
|---|---|---|---|
| Modifications by | Modifications by | Sequential Numbering | |
| EU Numbering | Kabat Numbering | (SEQ ID NO: 15) | Effects |
| L235E | L248E | L115E | Reduces FcγR binding; reduces ADCC |
| F234A/L235A | F247A/L248A | F114A/L115A | Reduce FcγR and C1q binding; reduced |
| | | | ADCC, ADCP and CDC |
| S228P/L235E | S241P/L248E | S108P/L115E | Reduce FcγR binding; reduced effector |
| | | | functions |
| S228P/F234A/L235A | S241P/F247A/L248A | S108P/F114A/L115A | Reduced binding to FcγRI, IIa and IIIa; |
| | | | reduced ADCC and CDC |

ii. Other Modifications of Fc portions

The Fc portion also can be modified to increase binding to inhibitory FcγRs, which results in the suppression of the immune response. Therapeutic antibodies with immunosuppressive Fc modifications are advantageous for the treatment of inflammatory diseases. These mutations can be incorporated into the Fc portions of constructs herein that are intended for treatment of diseases and conditions with an inflammatory component or etiology or involvement. For example, the immunosuppressive version of an anti-CD19 antibody (XmAb5871; Xencor), containing the mutations S267E/L328F (by EU numbering), binds inhibitory FcγRIIb with ~430-fold increased affinity, and depletes CD19⁺B-cells in patients with systemic lupus erythematosus (SLE). The same mutations, when introduced into a humanized anti-IgE antibody (XmAb7195; Xencor), prevent the binding of IgE to its high-affinity receptor (FcγRI) that is present on basophils and mast cells, increases affinity for FcγRIIb by ~430-fold, and is used for the treatment of allergies, including allergic asthma. The anti-CD3 antibody TRX4 (Tolerx), containing the aglycosylating Fc mutation N297A (by EU numbering), suppresses pathogenic T-cells and restores normal Treg cell activity in type-1 diabetes (autoimmune) patients (see, e.g., Saxena et al. (2016) Front. Immunol. 7:580).

An additional example is the monomeric IgG1 Fc (mFc), containing the mutations L351S/T366R/L368H/P395K (by EU numbering), which binds FcRn and exhibits similar in vivo half-life to dimeric Fc, and selectively binds FcγRI with high affinity, but does not bind FcγRIIIa, abrogating Fc-mediated cytotoxicity, including ADCC and CDC. FcγRI is expressed on inflammation-related cells, such as inflammatory macrophages. Targeting this receptor can be used for the treatment of chronic inflammatory diseases, such as arthritis, multiple sclerosis and cancer. The variant mFc, when fused to the Pseudomonas exotoxin A fragment (PE38), kills FcγRI macrophage-like U937 cells. Neither the variant mFc, nor the fusion protein, exhibits any cytotoxicity (ADCC or CDC) in vitro (see, e.g., Ying et al. (2014) mAbs 6(5):1201-1210).

Modifications that increase binding to, or that confer selective binding to, inhibitory FcγRIIb, and/or FcγRI but not FcγRIIIa, can be engineered into the IgG Fc regions in the TNFR1 antagonists and TNFR1 antagonist/TNFR2 agonist constructs provided herein. These modifications include, but are not limited to, one or more of S267E, N297A, L328F, L351S, T366R, L368H, P395K, S267E/L328F, L351S/T366R/L368H/P395K, and combinations thereof, by EU numbering. Table 11, below, shows the corresponding replacements by Kabat numbering, and by sequential numbering, with reference to the sequence of the IgG heavy chain constant domain set forth in SEQ ID NO:9.

TABLE 11
IgG1 Fc Modifications that Increase Binding to Inhibitory FcγRIIb

| | Modifications | Modifications by |
|---|---|---|
| Modifications by EU | by Kabat | Sequential Numbering |
| Numbering | Numbering | (SEQ ID NO: 9) |
| S267E | S280E | S150E |
| N297A | N314A | N180A |
| L328F | L347F | L211F |
| L351S | L372S | L234S |
| T366R | T389R | T249R |
| L368H | L391H | L251H |
| P395K | P423K | P278K |
| S267E/L328F | S280E/L347F | S150E/L211F |
| L351S/T366R/L368H/ | L372S/T389R/ | L234S/T249R/L251H/P278K |
| P395K | L391H/P423K | |

d. Additional Activity Modifiers—Fusion Proteins that Include Portions or Entire Polypeptides that Increase Serum Half-Life

Properties of the constructs can be altered by adding full-length polypeptides or portions thereof that increase serum half-life, but that do not substantially or do not alter the interaction of a construct with TNFR1 and/or TNFR2. This includes albumination and other such modifications (see discussion above regarding half-life extenders; reviewed in, Strohl (2015) BioDrugs 29(4):215-239, see also, Tan et al. (2018) Current Pharmaceutical Design 24:4932-4946).

### G. Assessing TNFR1 Antagonist and TNFR1 Antagonist/TNFR2 Agonist Construct Activity and Efficacy

If or as necessary, the constructs provided herein can be assessed for activity and efficacy using any assays, in vivo and/or in vitro, known to those of skill in the art, to assess properties of the constructs and/or suitability for treatment of particular diseases, disorders, or conditions. These assays also can be used to monitor treatment and/or predict response or select subjects for treatment. Exemplary assays are described in sections that follow.

In general, the antagonist constructs herein are those that are non-competitive; they generally are the constructs that lock the receptor in an inactive conformation, which as discussed above, means that selecting for high affinity is of lesser importance than selecting for antagonist activity.

### 1. Disease Activity Score (DAS28)

The 28-joint-count Disease Activity Score (DAS28; or Disease Activity Score of 28 joints) is a measure of disease activity in rheumatoid arthritis (RA), and is a simplification of the original DAS score, which requires 44 joints to be counted. The 28 joints that are counted include proximal interphalangeal joints (10 joints), metacarpophalangeal joints (10 joints), wrists (2), elbows (2), shoulders (2) and knees (2). The DAS28 is indicative of RA disease activity and response to treatment, and thus, is used in clinical trials for the evaluation of therapeutics for RA. The DAS28 is based on a count of 28 swollen and tender joints, with a score ranging from 0-10, with higher values indicating higher disease activity. In addition to counting the number of swollen and tender joints (out of the 28), the DAS28 includes a measurement of the erythrocyte sedimentation rate (ESR) or C reactive protein (CRP), which are acute phase reactants/blood markers of inflammation, as well as a general health (GH) assessment, representing the patient's self-assessment of disease activity, scored on a 100 mm visual analog scale (VAS), with a value of 0 meaning “no activity” and a value of 100 meaning “highest activity possible.” The DAS28 usually is combined with other measurements of disease severity, such as pain and grip strength, and physical function is assessed using a Health Assessment Questionnaire (HAQ).

To calculate DAS28 values, using ESR or CRP levels, the following formulas are used, respectively:

$$DAS 28 ( ESR ) = 0.56 \times √ ( TJC 28 ) + 0.28 \times √ ( SJC 28 ) + 0.014 \times GH + 0.7 \times ln ( ESR ) ;$$

$$DAS 28 ( CRP ) = 0.56 \times √ ( TJC 28 ) + 0.28 \times √ ( SJC 28 ) + 0.014 \times GH + 0.36 \times ln ( CRP + 1 ) + 0.96 ;$$

where TJC=tender joint count and SJC=swollen joint count.

A value of <2.6 indicates remission, value of <3.2 (>2.6 but ≤3.2) indicates low disease activity, a value of >3.2 but ≤5.1 indicates moderate disease activity, and a value of greater than 5.1 indicates high disease activity (i.e., active disease). An improvement (i.e., reduction in DAS28 score/value) of ≥1.2 indicates a good response/improvement; an improvement of >0.6 to ≤1.2 indicates a moderate response; and a DAS28 decrease of ≤0.6 indicates no improvement (see, e.g., Prevoo et al. (1995) Arthritis & Rheumatism 38(1):44-48; Wells et al. (2009) Ann. Rheum. Dis. 68:954-960).

The therapeutic efficacy of the selective TNFR1 antagonists, TNFR2 agonists, and/or bi-specific constructs containing the combination thereof, provided herein, can be evaluated by calculating the DAS28(ESR) or DAS28(CRP) before, during, and after treatment.

### 2. SOMAscan® Proteomic Analysis and Other Proteomic Tools for Quantifying Analytes

The SOMAscan® proteomic assay (SomaLogic, Inc.; Boulder, CO.) is an aptamer-based multiplexed, sensitive, quantitative and reproducible proteomic tool that can simultaneously measure the quantities of more than 5,000 protein analytes in a sample, such as serum, plasma or cerebrospinal fluid, as small as 150 μL in volume. Other biological matrices, such as cell culture supernatant, cell and tissue lysates, synovial fluid, and bronchoalveolar and nasal lavage, also can be used. Due to its ability to quantify a broad range or protein targets simultaneously, the SOMAscan® assay is optimized for protein biomarker discovery, and has been used to identify biomarker signatures associated with several diseases, including, for example, non-small cell lung cancer, Alzheimer's diseases, cardiovascular disease and inflammatory bowel disease. The SOMAscan® assay can be used to determine the protein signatures of, for example, RA patients, by analyzing samples taken before and after the initiation of treatment with the TNFR1 antagonist, TNFR2 agonist, and bispecific constructs provided herein. In this manner, the responses in patients can be monitored at an early time point in treatment and throughout treatment.

The SOMAscan® assay employs protein-capture reagents, known as Slow Off-rate Modified Aptamer (sold as SOMAmer® aptamers) reagents, which are short, single-stranded DNA-based protein affinity reagents constructed with chemically modified nucleotides that mimic amino acid side chains, have slow off-rates, and allow specific, high affinity binding to protein targets. The assay measures proteins in their native, folded conformations (i.e., tertiary structure), and does not detect unfolded and denatured (i.e., inactive) proteins. For the SOMAscan® assay, a SOMAmer®-protein binding step is followed by a series of partitioning and wash steps, whereby proteins in a biological sample are quantified by transforming each individual protein concentration into a corresponding SOMAmer® reagent concentration (SOMAmer®-based DNA signal), which then is quantified by standard DNA detection techniques, such as microarrays or qPCR. The assay takes advantage of SOMAmer reagents' dual nature as protein affinity-binding reagents with defined three-dimensional structures, and as containing unique nucleotide sequences recognizable by specific DNA hybridization probes.

SOMAmer® reagents are prepared with three tags, and contain a fluorophore linked to biotin via a photocleavable linker. Briefly, for the assay, the biological sample of interest is diluted, and then incubated with the respective SOMAmer® reagent mixes that are pre-immobilized onto streptavidin (SA)-coated beads. The SOMAmer® reagents bind to proteins in the biological sample, and the beads are washed to remove unbound proteins. Any non-specific complexes that form possess fast off-rates. Proteins that remain bound to their cognate SOMAmer® reagents are tagged using an NHS-biotin reagent, and a polyanionic competitor solution is added that breaks up any non-specific complexes. Protein-SOMAmer® complexes and unbound (free) SOMAmer® reagents are released from the streptavidin beads by cleaving the photocleavable linker using ultraviolet light. The photo-cleavage eluate, which contains all SOMAmer® reagents (some bound to biotin-labeled protein and some free), then is incubated with a second streptavidin-coated bead that binds the biotinylated proteins and the biotinylated protein-SOMAmer® complexes, and unbound material is removed by subsequent washing steps. In the final elution step, protein-bound SOMAmer® reagents are released from their cognate proteins using denaturing conditions, and the SOMAmer® reagents are quantified by standard DNA quantification techniques, such as by hybridization to custom DNA microarrays and measurement of the fluorophore tag. The data are reported in relative fluorescent units (RFUs), following normalization and calibration, and the measured SOMAmer® reagent signals correlate with the protein levels found in the biological sample (see, e.g., Gold et al. (2010) PLoS ONE 5(12):e15004; Candia et al. (2017) Sci. Reports 7:14248; Tanaka et al. (2018) Aging Cell. 17:e12799).

3. Transcriptome Analysis to Predict Responsiveness to Therapy and to Select Subjects Likely to Benefit from Treatment

Traditional anti-TNF therapies, i.e., TNF blockers, such as etanercept, infliximab and others, are met with ~30% non-responsiveness in RA patients. There, however, are clinical markers that can predict the efficacy of these anti-TNF therapies. The analysis of genes that are differentially expressed following anti-TNF therapy with etanercept, using global transcriptome analysis to determine RNA expression signatures in peripheral blood mononuclear cells (PBMCs), has been performed. Similar transcriptome analyses can be performed to evaluate the efficacy of the TNFR1 antagonists and the TNFR1 antagonist/TNFR2 agonist constructs herein, and/or to evaluate patient responsiveness to them.

In an exemplary protocol, blood samples are obtained from patients before and after treatment, and PBMCs are separated using a Ficoll density gradient, after which the populations of CD3⁺, CD14⁺, CD19⁺ and CD56⁺cells are evaluated using flow cytometry. Total RNA then is extracted, for example, using the Qiagen RNeasy® kit, and microarray analysis is used (e.g., using Affymetric® microarray technology) to analyze the expression profiles of tens of thousands of known genes in PBMCs to identify the profiles of responders/non-responders. The gene expression profiles can be determined at an early stage of treatment to quickly identify those who will be nonresponders. For example, by using this method to identify reliable biomarkers for predicting the therapeutic efficacy of etanercept in RA patients, gene pairs with a prediction accuracy of >89%, and gene triplets with a prediction accuracy of >95%, were identified. These include, for example, genes involved in TNF signaling via the NF-κB pathway, genes involved in NF-κB-independent signaling, and genes involved in the regulation of cellular and oxidative stress responses. For example, the gene triplets identified include TNFAIP3, encoding TNF-α-induced protein 3, a zinc finger protein shown to inhibit NF-κB activation; PDE4B, encoding a (cAMP)-specific cyclic nucleotide phosphodiesterase that is involved in NF-κB-independent signal transduction; and RAPGEF1, encoding Rap guanine nucleotide-exchange factor 1, an activator of RAS signaling. The expression of all three of these genes was downregulated 3 days following administration of etanercept in responders compared to in nonresponders. Other genes evaluated include CCL4, CXCR4, CCL3, PIGO, FSD1, RUNX1, LGALS13, PTPRD, IL1B, ADAM12, and HCG4P6 (see, e.g., Koczan et al. (2008) Arthritis Research & Therapy 10:R50).

The expression levels of a subset of genes of interest can be measured by quantitative real-time PCR (RT-PCR) using pre-designed primers and probes, to validate the results obtained with microarray analysis. To calculate the change in gene expression of selected genes, the ΔΔC_{T} method can be used, whereby the threshold cycle (C_{T}) values for specific mRNA expression in a sample are normalized to the C_{T} values of, for example, GAPDH mRNA, in the sample, and the gene expression change (ΔΔC_{T}) is defined by the difference in the C_{T} values after and before treatment (see, e.g., Koczan et al. (2008) Arthritis Research & Therapy 10:R50).

### 4. L929 Cytotoxicity Assay

TNFR1-mediated processes and cellular responses can be determined, for example, by evaluating TNF-induced cell death with an L929 cytotoxicity assay, whereby the TNFR1 antagonist inhibits TNF-induced cytotoxicity. Briefly, L929 mouse fibroblasts are plated in microtiter plates and incubated overnight with the TNFR1 antagonist, 100 μg/ml TNF and 1 mg/ml actinomycin D. Cell viability is measured by reading absorbance at 490 nm following incubation with 3-(4,5-dimethylthiazol-2-yl)-5-(3-carboxymethoxyphenyl)-2-(4-sulfophenyl)-2H-tetrazolium (MTS). The TNFR1 antagonist decreases TNF-mediated cytotoxicity, leading to an increase in absorbance, as compared to a TNF only control.

### 5. HeLa IL-8 Assay

The activity of the TNFR1 antagonists can be determined using a HeLa IL-8 assay, where the ability of the antagonists to neutralize TNF-induced IL-8 secretion in HeLa cells is evaluated. Briefly, HeLa cells are plated in microtiter plates overnight, in the presence of varying concentrations of TNFR1 antagonist and 300 μg/ml TNF. The supernatant then is aspirated off, and the concentration of IL-8 is measured using a sandwich ELISA. TNFR1 antagonist activity decreases IL-8 secretion into the supernatant, compared to a TNF only control.

### 6. HUVEC Assay

A human umbilical cord vein endothelial cell (HUVEC) assay can be used to determine the activity of the TNFR1 antagonists herein. Treatment of HUVECs with TNF results in the upregulation of VCAM-1 expression on the cells, which can be determined, for example, by ELISA. Since TNFR1 antagonists inhibit the action of TNF, VCAM-1 expression is reduced in HUVECs in the presence of the antagonists. The level of inhibition of TNF-induced VCAM-1 expression by the TNFR1 antagonist then is determined by plotting the concentration of the antagonist against the percent inhibition of VCAM-1 expression.

In accord with a protocol for this assay, HUVECs are cultured overnight, and then incubated with the TNFR1 antagonist for 1 hour, followed by stimulation with TNF (1 ng/mL) for 23 hours. Cells incubated with medium only are used as a negative control, and cells incubated with TNF only are used as a positive control. Cell culture supernatants a then are aspirated, and the cells are washed three times with ice cold PBS, and lysed by the addition of ice-cold Tris-Glycerol lysis buffer (40 mM Tris, 274 mM NaCl, 2% Triton-X-100, 20% glycerol, 50 mM NaF, 1 mM Na₃VO₄, 1× protease inhibitor tablet per 10 mL), then incubated for 15 mins on ice. The cell lysates then are used in a VCAM-1 sandwich ELISA. To calculate inhibition of TNF-induced VCAM-1 expression, the % inhibition of maximal VCAM-1 expression (i.e., as measured in the positive control)=

{100−[(OD value at antagonist conc.)/(OD value of positive control)]}×100.

The EC₅₀ values then are determined by plotting antagonist concentration against percentage inhibition, for example, using available software such as GraphPad Prism software.

### 7. Quantification and Evaluation of Treg Cell Activity

To determine the effects of the TNFR1 antagonist and TNFR1 antagonist/TNFR2 agonist constructs on Tregs, the numbers of Tregs can be quantified before and after treatment, as well as during treatment, by isolating peripheral blood mononuclear cells (PBMCs) from blood samples, such as by using Ficoll-Paque methods, followed by monoclonal antibodies (mAbs) and paramagnetic beads, or other similar methods known in the art, to isolate CD4⁺CD25⁺Tregs, as well as CD4⁺CD25⁻ non-regulatory T cells. Using flow cytometry, and immunostaining with mAbs against CD4 and CD25 (for Tregs), or CD4 and CTLA-4 (for non-regulatory T cells), the numbers of each cell type can be quantified (see, e.g., Vigna-Perez et al. (2005) Clin. Exp. Immunol. 141(2):372-380).

CD4⁺CD25⁺Tregs suppress the proliferation of CD4⁺CD25⁻ T cells. To test for the activity of Tregs from treated patients, a cell proliferation assay can be used, in which the Tregs and T cells are cultured together for 48 hours with phytohemagglutinin (PHA, to stimulate T cells). ³H-TdR (tritiated thymidine) is added for the last 12 hours of culture, and cells then are harvested, and proliferation is determined using a liquid scintillation counter. CD4⁺CD25⁻ T cells, cultured alone, are used as a control, and results are expressed in terms of the stimulation index (SI) of cell proliferation, which is calculated using the formula:

SI=(cpm of cells with PHA)÷(cpm of cells cultured with medium only),

where cpm is counts per minute, as determined by the counted radioactivity (see, e.g., Vigna-Peréz et al. (2005) Clin. Exp. Immunol. 141(2):372-380).

To test for immune reactivity against M. tuberculosis, PBMCs are cultured for 72 hours in complete medium, in the presence of a whole protein extract of the bacterium. ³H-TdR (tritiated thymidine) is added for the last 12 hours of culture, and cells then are harvested, and proliferation is determined using a liquid scintillation counter. Results are expressed as the stimulation index, as described above. For in vivo reactivity against M. tuberculosis, a standard PPD (purified protein derivative) skin test can be used (see, e.g., Vigna-Perez et al. (2005) Clin. Exp. Immunol. 141(2):372-380).

### 8. Evaluation of Binding Properties of the TNFR1 Antagonist Constructs

The specific binding of an antibody or antibody fragment or multi-specific constructs, such as the constructs provided herein, to TNFR1 and/or TNFR2 (e.g., human TNFR1 and/or TNFR2) can be assessed by any of a variety of known methods. The affinity can be represented quantitatively by various metrics, including the concentration of the TNFR1 antagonist, TNFR2 agonist, or multi-specific construct, needed to achieve half-maximal potentiation of TNFR1 and/or TNFR2 signaling in vitro (EC₅₀), and the equilibrium constants (K_{D}) of the antagonist-TNFR1 and/or agonist-TNFR2 complex dissociation. The equilibrium constant, K_{D}, which describes the interaction of TNFR1 or TNFR2 with a binder, such as a construct (binder) provided herein, is the chemical equilibrium constant for the dissociation reaction of a TNFR1-binder construct complex or TNFR2-binder complex into solvent-separated TNFR1 or TNFR2 and binder molecules that do not interact with one another.

The TNFR1 antagonists, TNFR2 agonists, and multi-specific constructs also can be characterized by a variety of in vitro binding assays. Examples of experiments that can be used to determine the KD or EC₅₀ include, for example, surface plasmon resonance (SPR, e.g., BIAcore™ analysis), isothermal titration calorimetry, fluorescence anisotropy, and ELISA-based assays, among others. ELISA is a particularly useful method for analyzing antibody activity, as such assays typically require minimal concentrations of antibodies. A common signal that is analyzed in a typical ELISA assay is luminescence, which is typically the result of the activity of a peroxidase conjugated to a secondary antibody that specifically binds a primary antibody (e.g., a TNFR1-antagonist or TNR1 antagonnist-TNFR2-agonist bi-specific construct provided herein).

The kinetics of association and dissociation of the TNFR1 antagonist with TNFR1, or the TNFR2 agonist with TNFR2 can be quantitatively characterized, for example, by monitoring the rate of antibody-antigen complex formation according to established procedures. For example, one can use surface plasmon resonance (SPR) to determine the rate constants for the formation (kₒₙ) and dissociation (k_{off}) of an antagonist-TNFR1 or agonist-TNFR2 complex. The equilibrium constant (K_{D}) can be determined from these data, since the equilibrium constant of this unimolecular dissociation can be expressed as the ratio of the k_{off} to kₒₙ values. SPR is a technique that is advantageous for determining kinetic and thermodynamic parameters of receptor-antibody (or other binder) interactions, since the experiment does not require that one component be modified by attachment of a chemical label. Rather, the receptor typically is immobilized on a solid metallic surface which is treated in pulses with solutions of increasing concentrations of antibody or binder (i.e., TNFR1 antagonist or TNFR2 agonist, or bi-specific constructs thereof). Antibody-receptor binding induces distortion in the angle of reflection of incident light at the metallic surface, and this change in refractive index over time as antibody is introduced to the system can be fit to established regression models known in the art in order to calculate the association and dissociation rate constants of an antibody-receptor interaction.

### 9. Antibody-Dependent Cellular Cytotoxicity (ADCC) and Complement-Dependent Cytotoxicity (CDC) Assays

Antibody-dependent cellular cytotoxicity (ADCC) and complement-dependent cytotoxicity (CDC) assays can be used to evaluate the immune effector functions/cytotoxicity of the TNFR1 antagonist, TNFR2 agonist, and multi-specific constructs provided herein that contain an Fc monomer or dimer. In general the Fc portion(s) is/are modified to eliminate or substantially reduce (to eliminate or reduce adverse side effects to a tolerable level) ADCC or ADCC and CDC effector functions. Such assays are well known in the art (see, e.g., Ying et al. (2014) mAbs 6(5):1201-1210). For example, for an exemplary ADCC assay, mesothelin-negative A431 or mesothelin-positive H9 cells are incubated with the TNFR1 antagonist, TNFR2 agonist, or multi-specific constructs provided herein for 30 min, followed by the addition of the target cells to wells containing the effector cells (e.g., PBMCs), at an effector to target cell ratio of 50:1. After a 24 hour incubation, the lysis of the target cells is measured using the CytoTox-ONE Homogenous Membrane Integrity Assay (Promega), according to the manufacturer's protocol.

For an exemplary CDC assay, A431 and H9 cells are washed in serum-free RPMI and density adjusted to 1 million/mL in serum-free RPMI. 50 μL of cell suspension then is incubated with 50 μL of TNFR1 antagonist, TNFR2 agonist, or multi-specific construct dilution in RPMI. A negative control contains 50 μL of cell suspension with 50 μL of RPMI, and a positive control contains target cells lysed with 1% Triton X-100 in a final volume of 150 μL. Fresh human plasma is diluted in PBS (1:4) and clarified with centrifugation, and then 50 μL of diluted plasma is added to each cell/construct mixture and incubated in 96-well plates at 37° C., to allow for complement-mediated cell lysis. Following a 3 hour incubation, 100 μL of supernatant is transferred to a white plate, and 100 μL substrate from the CytoTox-ONE Homogenous Membrane Integrity Assay Kit (Promega) is added. The plate then is incubated for 10 min at room temperature, and fluorescent signals are read using a fluorimeter, with an excitation wavelength of 530 nm and emission wavelength of 590 nm. The CDC of target cells is expressed as the percent of the experimental sample to the positive control.

### 10. Disease Models

The selective TNFR1 antagonists, TNFR2 agonists, and multi-specific constructs, provided herein, can be assessed in any clinically relevant disease model known to one of skill in the art, to determine their effects on autoimmune and inflammatory and other diseases or disorders that are mediated by or involve TNF in their etiology. Exemplary disease models include, but are not limited to, collagen-induced arthritis (CIA), rheumatoid arthritis synovial membrane mononuclear cell cultures, the Tg197 mouse model of arthritis, AARE mouse models of arthritis/IBD, the mouse dextran sulfate sodium (DSS) induced model of IBD, and the experimental autoimmune encephalomyelitis (EAE) model for multiple sclerosis. Other models are known to those of skill in the art. See, e.g., Malaviya et al. (2017) Pharmacol Ther. 180:90-98, which provides numerous models for testing the constructs for treating inflammatory lung diseases; Feldmann et al. (2020) Lancet 395:1407-1409, directed to use of anti-TNF therapies, and models, treatments for COVID-19; Shi et al. (2013) Crit. Care 17(6):R301, use of anti-TNF therapies for treating H1N1, and models for viral infection; and Orti-Casan et al. (2019) Front. Neurosci. 13:49, which describes that activating TNFR2 for treating Alzheimer's disease is advantageous, evidencing the approach herein that TNF blockers, which inhibit TNFR1 and TNFR2, are problematic. The following is a non-exhaustive discussion of diseases, disorders, and conditions that can be treated with constructs provided herein, and exemplary models for each. These are exemplary; the skilled person can select appropriate models for a particular construct and targeted disease, disorder, or condition. Because the anti-TNFR1 and TNFR2 antagonist/agonist constructs provided herein are intended for use for targeting human TNFR1/TNFR2, they are not expected to work as well in reacting/interacting with TNFR1/TNFR2 from non-human, particularly non-primate, species. For testing, in non-human models, such as rodent models, the model, such as a mouse model, is transgenic for human TNFR1 and human TNFR2. They can be used against a background of murine TNFR1/2 knockout mice for in vivo models of inflammation and autoimmune disease. Alternatively, severely immunocompromised mice (such as, NOD/NSG mice) mouse transplanted with human CD34+ stem cells, can be used for this purpose. Alternatively, human rheumatoid arthritis synovial cells can be transplanted into immunodeficient mice, resulting in RA-like inflammation (see, e.g., Schinnerling et al. (2019) Front Immunol. 10:203, for a description of such models).

a. Collagen-Induced Arthritis (CIA)

Type II collagen-induced arthritis (CIA) can be induced in mice as a model of autoimmune inflammatory joint disease that is histologically similar to RA, and is characterized by inflammatory synovitis, pannus formation, and erosion of cartilage and bone. To induce CIA, bovine type II collagen (B-CII), in the presence of complete Freund's adjuvant, is injected intradermally at the base of the tail. After 21 days, mice can be re-immunized using the same protocol. To examine the effects of the selective TNFR1 antagonists, TNFR2 agonists, and multi-specific constructs provided herein, 3 weeks following the initial challenge with B-CII, or upon the development of signs of arthritis, selective TNFR1 antagonists, TNFR2 agonists, or multi-specific constructs, or control can be administered intraperitoneally twice weekly for 3 weeks. Mice can be sacrificed 7 weeks following the initial immunization for histologic analysis.

To assess the therapeutic effect of the constructs on established disease, they can be administered daily for a total of 10 days following the onset of clinical arthritis in one or more limbs. The degree of swelling in the initially affected joints can be monitored by measuring paw thickness using calipers. Serum can be drawn from mice for the measurement of proinflammatory cytokines and chemokines, such as, for example, granulocyte-macrophage colony stimulating factor (GM-CSF), interleukin-10 (IL-10), IL-1β, IL-6, IL-8, RANTES (CCL5) and monocyte chemoattractant protein 1 (MCP-1; also known as CCL2).

In another example, primate models are available for RA treatments. Response of tender and swollen joints (e.g., as measured by clinical arthritic scores) can be monitored in subjects treated with recombinant therapeutic TNFR1 antagonistic, TNFR2 agonistic, or bispecific constructs, and controls, to assess therapeutic efficacy and treatment.

b. Rheumatoid Arthritis Synovial Membrane Mononuclear Cell Cultures

Human rheumatoid arthritis (RA) synovial membrane mononuclear cells (MNCs), which express TNFR1 and TNFR2, also can be used to test the therapeutic efficacy of the constructs provided herein. The RA synovial membrane MNCs can be obtained from RA patients undergoing joint replacement surgery, and cultured ex vivo to evaluate RA synovial cell cytokine production and regulation. RA synovial membrane MNC cultures produce inflammatory cytokines and chemokines spontaneously, in the absence of exogenous stimulation; antibody-mediated neutralization of TNF, and the selective blockade of TNFR1, such as by constructs provided herein, in these cultures inhibits the production of proinflammatory cytokines and chemokines, such as GM-CSF, IL-10, IL-1β, IL-6, IL-8, RANTES (CCL5) and MCP-1 (CCL2).

In an exemplary assay, to prepare the RA synovial membrane MNCs, RA synovial membrane tissues are dissected into small pieces, incubated for 1 hour at 37° C. with 5 mg/ml collagenase A and 0.15 mg/ml DNase in RPMI 1640, after which the digested tissue is passed through a 170 μm filter and washed 3 times with RPMI 1640 containing 100 units/ml of streptomycin, 100 μg of penicillin, and 10% FCS.

Heterogeneous RA synovial membrane MNCs then are used without passage. For ex vivo cell culture, single-cell suspensions of RA synovial membrane MNCs are cultured in RPMI 1640 medium with 5% FCS in 96-well flat-bottomed plates (2×10⁵ cells/well) for 2-5 days at 37° C. and 5% CO₂, in the presence or absence of the TNFR1 antagonistic, TNFR2 agonistic, or bispecific constructs, or control. The supernatant then is collected and used immediately, or stored at −20° C., for analysis by cytokine and chemokine ELISAs (see, e.g., Schmidt et al. (2013) Arthritis & Rheumatism 65(9):2262-2273). Alternatively, the cytokines in culture supernatants can be quantified by cytokine bead array analysis.

c. Tg197 Mouse Model of Arthritis

The Tg197 transgenic strain of mice, a mouse model of erosive arthritis, is a well-established animal model of RA. Tg197 mice are human TNF-transgenic C57BL/6 mice that overexpress human TNF and develop a symmetric polyarthritis with pannus formation, bone destruction, and cartilage damage that is characteristic of human RA. In addition to displaying features of chronic destructive joint disease, this model exhibits symptoms, such as enthesitis or bilateral sacroiliitis, which are characteristic of other inflammatory diseases, such as spondyloarthritis (Blüml et al. (2010) Arthritis & Rheumatism 62(6):1608-1619). Tg197 mice develop arthritis with 100% penetrance and provide a fast in vivo model for evaluating human therapeutics that target RA. For example, the Tg197 mouse model was used to evaluate the therapeutic efficacy of infliximab (originally sold as Remicade®), the first anti-TNF therapeutic successfully applied in the clinic, and is recommended by the FDA for screening potential anti-RA therapeutics.

Tg197 mice carry five copies of a human TNF gene construct, in which the 3′-region, containing the 3′-untranslated and 3′-flanking sequences, is exchanged with the 3′-region of the human P-globin gene. This gene construct is microinjected into mouse zygotes, creating an in vivo model of deregulated TNF gene expression, since a set of highly conserved UA-rich sequences at the 3′-untranslated region of TNF mRNA is critical for the regulation of mRNA stability and translation efficiency (see, e.g., Keffer et al. (1991) EMBO J. 10(13):4025-4031).

### d. ΔΔRE Mouse Model of Arthritis/IBD

Mice with a deletion in the 3′ AU-rich elements (AREs) of the TNF mRNA (Tnf^{ΔARE}) overproduce TNF and develop an inflammatory bowel disease that is histopathologically similar to Crohn's disease, at between 4-8 weeks of age. The mice also develop clinical signs of RA. The efficacy of the TNFR1 antagonists, TNFR2 agonists, and bispecific constructs, provided herein, can be evaluated by assessing the inhibitory effects on the Crohn's-like pathology and arthritis in Tnf^{ΔARE} mice following intraperitoneal injection (see, e.g., U.S. Pat. No. 9,028,822).

e. Humanized TNF/TNFR2 Mice

A limitation in the development of therapeutics targeting the TNF/TNFR2 signaling pathway is the lack of preclinical animal models, as many human anti-TNF therapeutics do not interact with murine TNF or TNFR2, and human TNF can bind to and engage murine TNFR1, but not TNFNR2. Humanized TNF/TNFR2 mice, which carry a functional human TNF-TNFR2 (hTNF-hTNFR2) signaling module, can be used to evaluate therapeutics, such as agonistic and antagonistic antibodies constructs human TNF or human TNFR2, in various models of autoimmunity. Such TNF/TNFR2 doubly humanized mice can be used, for example, to evaluate the TNFR2 agonist constructs provided herein.

Humanized TNF/TNFR2 mice can be generated as described in Atretkhany et al. (2018) Proc. Natl. Acad. Sci. U.S.A. 115(51):13051-13056. Briefly, human TNFR2 knock-in (hTNFR2KI) and human TNF knock-in (hTNFKI) mice are generated using standard genetic engineering techniques. The hTNFKI mice, in which the human TNF gene has replaced the mouse TNF gene, then are crossed with the hTNFR2KI mice, containing a humanized TNFR2 ligand-binding portion, and then intercrossed to generate doubly humanized double homozygous hTNFKI×hTNFR2KI mice. To assess the role of TNFR2 signaling in particular cells, e.g., Tregs, two LoxP sites are inserted within the hTNFR2 locus, to allow for the conditional Cre-mediated ablation of the extracellular portion of TNFR2. For Treg-specific deletion of TNFR2, these mice are crossed with FoxP3-Cre transgenic mice (see, e.g., Atretkhany et al. (2018) Proc. Natl. Acad. Sci. U.S.A. 115(51):13051-13056).

### H. Methods of Producing Nucleic Acids Encoding Tnfr1 Antagonist Constructs and Tnfr1 Antagonist/Tnfr2 Agonist Constructs

TNFR1 antagonist polypeptides, TNFR2 agonist polypeptides, and TNFR1 antagonist/TNFR2 agonist polypeptide constructs, provided herein, that are polypeptides can be obtained by methods well known in the art for protein purification and recombinant protein expression, and also for recombinant antibody preparation. Constructs provided herein that include portions, such as linkers, that are not polypeptides can be prepared by chemical conjugation methods as appropriate. The polypeptide portions can be produced by standard recombinant technology, such as by expression in a suitable host (bacterial if no glycosylation is desired, or in eukaryotic cells, such as HEK293 and CHO cells, if glycosylated forms are desired). Active antibodies and antibody fragments have been produced in E. coli, but these often have aggregation and solubility problems because of improper folding, which can be remedied by further mutagenesis of the coding sequences (see, e.g., Kunz et al. (2018) Sci Rep. 8(1):7934).

Polypeptides also can be synthesized chemically. Fusion polypeptides can be synthesized by standard methods of recombinant production. The components, discussed above, of the various constructs can be separately synthesized, and combined using standard methods to produce the constructs.

Nucleic acid encoding the polypeptide constructs or polypeptide portions thereof, including modified or variant, including truncated forms, can be prepared from nucleic acid. Modified or variant polypeptides can be engineered from nucleic acid encoding wild type polypeptides using standard recombinant DNA methods. For example, modified TNF polypeptides, such as the TNF muteins, that selectively bind to TNFR1 or TNFR2, and/or that selectively antagonize TNFR1 or selectively agonize TNFR2, can be engineered from wild type TNF, such as by site-directed mutagenesis of the encoding DNA. Any methods known to those of skill in the art can be used. The following discussion and description in the Examples is exemplary.

### 1. Isolation or Preparation of Nucleic Acids Encoding TNFR1 Antagonist and TNFR2 Agonist Polypeptides

Nucleic acids encoding TNFR1 antagonist polypeptides, TNFR2 agonist polypeptides, and TNFR1 antagonist/TNFR2 agonist polypeptide constructs can be cloned or isolated using any available methods known in the art for cloning and isolating nucleic acid molecules. Such methods include polymerase chain reaction (PCR) amplification of nucleic acids and screening of libraries, including nucleic acid hybridization screening, antibody-based screening and activity-based screening. For example, when the polypeptides are produced by recombinant means, any method known to those of skill in the art for identification of nucleic acids that encode desired polypeptides can be used.

Nucleic acid molecules encoding the polypeptides herein can be synthetically produced, or can readily be isolated and sequenced, as needed, using conventional procedures (e.g., by using oligonucleotide probes that are capable of binding specifically to genes encoding the heavy and/or light chains of the antibody fragments, such as, e.g., single domain antibodies (dAbs), scFv fragments, and Fab antibody fragments). For example, any cell source known to produce or express a TNFR1 antagonist or TNFR2 agonist antibody or fragment(s) thereof can serve as a source of such DNA. In another example, once the sequence of the DNA encoding the TNFR1 antagonist or TNFR2 agonist antibody or fragment(s) thereof is determined, nucleic acid sequences can be constructed using gene synthesis techniques.

Methods for amplification of nucleic acids can be used to isolate nucleic acid molecules encoding a desired polypeptide, including for example, polymerase chain reaction (PCR) methods. Exemplary of such methods include use of a Perkin-Elmer Cetus thermal cycler and Taq polymerase (Gene Amp). A nucleic acid containing material can be used as a starting material from which a desired polypeptide-encoding nucleic acid molecule can be isolated. For example, DNA and mRNA preparations, cell extracts, tissue extracts, fluid samples (e.g., blood, serum, and saliva), and samples from healthy and/or diseased subjects can be used in amplification methods. The source, which generally will be from human sources, can be, if appropriate from any eukaryotic species including, but not limited to, vertebrate, mammalian, human, porcine, bovine, feline, avian, equine, canine, and other primate sources. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. If desired, degenerate primers can be used for amplification. Oligonucleotide primers that hybridize to sequences at the 3′ and 5′ termini of the desired sequence can be used as primers to amplify sequences by PCR from a nucleic acid sample. Primers can be used to amplify the entire full-length polypeptide, or a truncated sequence thereof, such as a nucleic acid encoding any of the TNFR1 antagonist and TNFR1 agonist polypeptides, as well as the TNFR1 antagonist/TNFR2 agonist polypeptide constructs, provided herein. Nucleic acid molecules generated by amplification can be sequenced and confirmed to encode the desired polypeptide or construct.

Mutagenesis techniques can be employed to generate further modified forms of a TNFR1 antagonist or TNFR2 agonist antibody or fragment(s) thereof and to produce modified forms of the activity modifier, such as Fc and hinge regions, and linker portions. The DNA also can be modified. For example, gene synthesis and routine molecular biology techniques can be used to effect insertion, deletion, addition or replacement/substitution of nucleotides. Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core polypeptide-coding DNA sequences. Additional nucleotide sequences specifying functional DNA elements, such as promoters, enhancers, and IRES sequences, can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. Additional nucleotide sequences, such as sequences specifying protein binding regions, also can be linked polypeptide-encoding nucleic acid molecules. Such regions include, but are not limited to, sequences that facilitate uptake of a polypeptide into specific target cells, or that otherwise alter or enhance the pharmacokinetics of the product of a synthetic gene.

Tags and/or other moieties can be added, for example, to aid in detection or affinity purification of the polypeptide. For example, additional nucleotide sequences, such as sequences of bases specifying an epitope tag or other detectable marker, also can be linked to polypeptide-encoding nucleic acid molecules. Exemplary of such sequences include nucleic acid sequences encoding a SUMO tag, or His tag, or Flag Tag.

It is understood that any of the amino acid sequences provided herein can be reverse-translated (also called back translated), using standard methods commonly used by those skilled in the art, to generate corresponding encoding nucleic acid sequences, which can be cloned into vectors and expressed to generate the constructs, including polypeptides, antibodies and antibody fragments, provided herein. For example, there are several online tools are available to convert protein sequences to encoding DNA sequences, such as bioinformatics.org/sms2/rev_trans.html; biophp.org/minitools/protein_to_dna/demo.php; vivo.colostate.edu/molkit/rtranslate/; ebi.ac.uk/Tools/st/emboss_backtranseq/; molbiol.ru/eng/scripts/01_19.html; and geneinfinity.org/sms/sms_backtranslation.html. Such reverse translated sequences can be inserted into any of the expression vectors provided herein for the expression and production of the provided antibodies or fragments. Anti-TFR1 and anti-TNFR2 antibodies, such as the TNFR1 antagonist and TNFR2 agonist constructs can be expressed as full-length proteins or less than full length proteins. For example, antibody fragments, such as, but not limited to, single domain antibodies (dAbs), scFv fragments, and Fab fragments, can be expressed.

The identified and isolated nucleic acids then can be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives or the pBluescript vector (Stratagene, La Jolla, CA). The insertion into a cloning vector can, for example, be accomplished by ligating the DNA fragment into a cloning vector which has complementary cohesive termini. Insertion can be effected using TOPO cloning vectors (Invitrogen, Carlsbad, CA).

If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and polypeptide gene can be modified by homopolymeric tailing.

Recombinant molecules can be introduced into host cells via, for example, transformation, transfection, infection, electroporation and sonoporation, so that many copies of the gene sequence are generated. In specific embodiments, transformation of host cells with recombinant DNA molecules that incorporate the isolated polypeptide gene, cDNA, or synthesized DNA sequence, enables generation of multiple copies of the gene. Thus, the gene can be obtained in large quantities by growing transformants, isolating the recombinant DNA molecules from the transformants and, when necessary, retrieving the inserted gene from the isolated recombinant DNA.

For expression of antibodies and fragments thereof, generally, a nucleic acid molecule encoding the heavy chain of an antibody is cloned into a vector, and a nucleic acid molecule encoding the light chain of an antibody is cloned into a vector. Methods for production of antibodies and portions thereof are well known (see, e.g., U.S. Pat. Nos. 4,816,567, 6,331,415, and 7,923,221, and numerous other seminal patents). The genes can be cloned into a single vector for dual expression thereof, or into separate vectors. If desired, the vectors also can contain further sequences encoding additional constant region(s) or hinge regions to generate other antibody forms. The vectors can be transfected and expressed in host cells. Expression can be in any cell expression system known to one of skill in the art. For example, host cells include cells that do not otherwise produce immunoglobulin protein, to obtain the synthesis of antibodies in the recombinant host cells. For example, host cells include, but are not limited to, simian COS cells, Chinese hamster ovary (CHO) cells, such as for example, CHO-DG44 (DHFR⁻) and FreeStyle™ CHO-S cells, Invitrogen), 293FS cells, HEK293 cells, NSO cells or other myeloma cells. Other expression vectors and host cells are described herein.

The constructs provided herein, including the TNFR1 antagonists, TNFR2 agonists and TNFR1 antagonist/TNFR2 agonist constructs, can be generated or expressed as full-length constructs or less than full-length, including, but not limited to, antigen-binding fragments, such as, for example, single domain antibody (dAb), Fab, Fab′, Fab hinge, F(ab′)₂, single-chain Fv (scFv), scFv tandem, Fv, dsFv, scFv hinge, scFv hinge (AE), diabody, Fd and Fd′ fragments. There are various techniques for the production of antibody fragments. For example, fragments can be derived via proteolytic digestion of intact antibodies (see, e.g., Morimoto and Inouye (1992) Journal of Biochemical and Biophysical Methods 24:107-117; Brennan et al. (1985) Science 229:81-83). Fragments also can be produced directly by recombinant host cells. For example, dAb, Fab, Fv and scFv antibody fragments can all be expressed in and secreted from host cells, such as E. coli, CHO cells or HEK293 cells, thereby facilitating production of large amounts of these fragments. F(ab′)₂ fragments can be produced by chemically coupling Fab′-SH fragments (see, e.g., Carter et al. (1992) Bio Technology, 10:163-167), or they can be isolated directly from recombinant host cell cultures. In some examples, the TNFR1 antagonist constructs include a single domain antibody (dAb; described, for example, in International Application Publication Nos. WO 2004/058820, WO 2004/081026, WO 2005/035572, WO 2006/038027, WO 2007/049017, WO 2008/149144, WO 2008/149148, WO 2010/094720, WO 2011/006914, WO 2011/051217, WO 2012/172070, WO 2012/104322, and WO 2015/104322; Enever et al., (2015) Protein Engineering, Design & Selection 28(3):59-66, U.S. Application Publication Nos. 2006/0083747, 2010/0034831, and 2012/0107330; and U.S. Pat. Nos. 9,028,817 and 9,028,822), a single-chain Fv fragment (scFv) (see, e.g., International Application Publication Nos. WO 2017/174586, and WO 2008/113515; see, also, Richter, F. Thesis, entitled “Evolution of the Antagonistic Tumor Necrosis Factor Receptor One-Specific Antibody ATROSAB,” Universitat Stuttgart, 2015; available from pdfs.semanticscholar.org/d8e7/8b87d76dce36225cid497939ef37445cfa8a.pdf), or a Fab fragment (see, e.g., International Application Publication Nos. WO 2017/174586 and WO 2008/113515; see, also, Richter, F. Thesis, entitled “Evolution of the Antagonistic Tumor Necrosis Factor Receptor One-Specific Antibody ATROSAB,” Universität Stuttgart, 2015; available from pdfs.semanticscholar.org/d8e7/8b87d76dce36225c1d497939ef37445cfa8a.pdf). dAb, Fv and scFv fragments have intact combining sites but are devoid of constant regions; thus, they are suitable for reduced non-specific binding during in vivo use. dAb and scFv fusion proteins can be constructed to attach an effector protein (e.g., an IgG Fc) at either the amino- or the carboxy-terminus of a dAb or scFv. The antibody fragment can also be a linear antibody (see, e.g., U.S. Pat. No. 5,641,870). Such linear antibody fragments can be monospecific or bispecific. Other techniques for the production of antibody fragments are known to one of skill in the art.

Upon expression, antibody heavy and light chains, or fragment(s) thereof, pair by interchain disulfide bonds to form a full-length antibody or fragment thereof. For example, for expression of a full-length Ig, sequences encoding the V_{H}-C_{H}1-hinge-C_{H}2-C_{H}3 can be cloned into a first expression vector, and sequences encoding the V_{L}-C_{L} domains can be cloned into a second expression vector. Upon co-expression, the full-length heavy and light chains are interlinked by disulfide bonds to generate a full-length antibody. In another example, to generate a Fab, sequences encoding a fragment containing the V_{H} and C_{H}1 regions can be cloned into a first expression vector, and sequences encoding the V_{L}-C_{L} domains can be cloned into a second expression vector. Upon co-expression, the heavy chain pairs with a light chain to generate a Fab monomer. Sequences of C_{H}I, hinge, C_{H}2 and/or C_{H}3 regions of various IgG sub-types are known to one of skill in the art (see, e.g., U.S. Publication No. 2008/0248028; see, also, SEQ ID NOs: 9, 11, 13, and 15). Similarly, sequences of C_{L}, lambda or kappa, also are known (see, e.g., U.S. Publication No. 2008/0248028; see, also, SEQ ID NOS: 17-22).

In addition to recombinant production, TNFR1 antagonist polypeptides, TNFR2 agonist polypeptides, and TNFR1 antagonist/TNFR2 agonist polypeptide constructs, provided herein, can be produced by direct peptide synthesis using well-known solid-phase techniques. In vitro protein synthesis can be performed using manual techniques or by automation. Automated synthesis can be achieved, for example, using the Applied Biosystems 431A Peptide Synthesizer (Perkin Elmer; Foster City, CA), in accordance with the instructions provided by the manufacturer. Various fragments of a polypeptide can be chemically synthesized separately and combined using chemical methods.

### 2. Generation of Mutant or Modified Nucleic Acids and Encoding Polypeptides

The modifications provided herein can be made by standard recombinant DNA techniques, such as are routine to one of skill in the art. Any method known in the art to effect mutation of any one or more amino acids in a target protein or polypeptide can be employed. Methods include standard site-directed mutagenesis (using, e.g., a kit, such as the QuikChange kit available from Stratagene) of encoding nucleic acid molecules, or solid-phase polypeptide synthesis methods.

### 3. Vectors and Cells

For recombinant expression of one or more of the desired polypeptides, such as any TNFR1 antagonist or TNFR2 agonist polypeptides, or TNFR1 antagonist/TNFR2 agonist polypeptide constructs, described herein, the nucleic acid molecule containing all or a portion of the nucleotide sequence encoding the polypeptide can be inserted into an appropriate expression vector, i.e., a vector that contains the necessary elements for the transcription and translation of the inserted polypeptide coding sequence. Also provided are vectors that contain nucleic acid molecules encoding the polypeptides. After insertion of the nucleic acid molecule(s), the vectors typically are used to transform host cells, for example, to amplify the nucleic acid for replication and/or expression thereof. In such examples, a vector suitable for high level expression is used. In other cases, a vector is chosen that is compatible with display of the expressed polypeptide on the surface of the cell The choice of vector can depend on the desired application. Many expression vectors are available and known to those of skill in the art for the expression of anti-TNFR1 and anti-TNFR2 antibodies or portions thereof, such as antigen-binding fragments. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows for selection and maintenance of the transformed cells. In some cases, a high copy number origin of replication can be used to amplify the copy number of the vectors in the cells. Vectors also generally can contain additional nucleotide sequences operably linked to the ligated nucleic acid molecule (e.g., His tag, Flag tag). For applications with antibodies, vectors generally include sequences encoding the constant region. Thus, antibodies or portions thereof also can be expressed as protein fusions. For example, a fusion protein can be generated to add additional functionality to a polypeptide. Examples of fusion proteins include, but are not limited to, fusions of a signal sequence, an epitope tag such as for localization, e.g., a His₆ tag or a myc tag, or a tag for purification, such as a GST tag, and/or a sequence for directing protein secretion and/or membrane association. Fusion proteins herein also include fusions of the TNFR1 antagonist and/or the TNFR2 agonist to a modified Fc region, the hinge region of an IgG, and/or a peptide linker, such as a GS linker.

A variety of host-vector systems can be used to express the protein coding sequence. These include, but are not limited to, mammalian cell systems infected with virus (e.g., vaccinia virus, adenovirus, and other viruses); insect cell systems infected with virus (e.g., baculovirus); microorganisms, such as yeast, containing yeast vectors; and bacteria transformed with bacteriophage, DNA, plasmid DNA, or cosmid DNA. The choice between eukaryotic expression systems and bacterial systems depends upon the desired post-translational modifications, such as glycosylation. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a nucleic acid molecule encoding a polypeptide, such as, for example, an antibody fragment or TNFR1 antagonist, or TNFR2 agonist, provided herein, as well as appropriate transcriptional/translational control signals. These methods can include in vitro recombinant DNA and synthetic techniques, and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

For example, expression of the construct polypeptides, such as the TNFR1 antagonists, TNFR2 agonists and TNFR1 antagonist/TNFR2 agonist constructs herein, can be controlled by any promoter/enhancer known in the art. Suitable bacterial promoters are well-known in the art and described herein below. Other suitable promoters for mammalian cells, yeast cells and insect cells are well-known in the art and some are exemplified below. Selection of the promoter used to direct expression of a heterologous nucleic acid depends on the particular application.

Promoters which can be used include, but are not limited to, eukaryotic expression vectors containing the SV40 early promoter (see, e.g., Benoist and Chambon (1981) Nature 290:304-310), the promoter contained in the 3′ long terminal repeat of Rous sarcoma virus (see, e.g., Yamamoto et al. (1980) Cell 22:787-797), the herpes thymidine kinase promoter (see, e.g., Wagner et al. (1981) Proc. Natl. Acad. Sci. U.S.A. 78:1441-1445), the regulatory sequences of the metallothionein gene (see, e.g., Brinster et al. (1982) Nature 296:39-42), and the cytomegalovirus (CMV) promoter; prokaryotic expression vectors such as the β-lactamase promoter (see, e.g., Jay et al. (1981) Proc. Natl. Acad. Sci. U.S.A. 78:5543), or the tac promoter (see, e.g., DeBoer et al. (1983) Proc. Natl. Acad. Sci. U.S.A. 80:21-25); see also “Useful Proteins from Recombinant Bacteria”: in Scientific American 242:79-94 (1980); plant expression vectors containing the nopaline synthetase promoter (see, e.g., Herrara-Estrella et al. (1984) Nature 303:209-213), the cauliflower mosaic virus 35S RNA promoter (see, e.g., Gardner et al. (1981) Nucleic Acids Res. 9(12):2871-2888), and the promoter of the photosynthetic enzyme ribulose bisphosphate carboxylase (see, e.g., Herrera-Estrella et al. (1984) Nature 310:115-120); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region, which is active in pancreatic acinar cells (see, e.g., Swift et al. (1984) Cell 38:639-646; Ornitz et al. (1986) Cold Spring Harbor Symp. Quant. Biol. 50:399-409; MacDonald (1987) Hepatology 7:425-515), insulin gene control region, which is active in pancreatic beta cells (see, e.g., Hanahan et al. (1985) Nature 315:115-122), immunoglobulin gene control region, which is active in lymphoid cells (see, e.g., Grosschedl et al. (1984) Cell 38:647-658; Adams et al. (1985) Nature 318:533-538; Alexander et al. (1987) Mol. Cell Biol. 7:1436-1444), mouse mammary tumor virus control region, which is active in testicular, breast, lymphoid and mast cells (see, e.g., Leder et al. (1986) Cell 45:485-495), albumin gene control region, which is active in liver (see, e.g., Pinckert et al. (1987) Genes and Devel. 1:268-276), alpha-fetoprotein gene control region, which is active in liver (see, e.g., Krumlauf et al. (1985) Mol. Cell. Biol. 5:1639-1648; Hammer et al. (1987) Science 235:53-58), alpha-1 antitrypsin gene control region, which is active in liver (see, e.g., Kelsey et al. (1987) Genes and Devel. 1:161-171), beta globin gene control region, which is active in myeloid cells (see, e.g., Magram et al. (1985) Nature 315:338-340; Kollias et al. (1986) Cell 46:89-94), myelin basic protein gene control region, which is active in oligodendrocyte cells of the brain (see, e.g., Readhead et al. (1987) Cell 48:703-712), myosin light chain-2 gene control region, which is active in skeletal muscle (see, e.g., Shani (1985) Nature 314:283-286), and gonadotrophic releasing hormone gene control region, which is active in gonadotrophs of the hypothalamus (see, e.g., Mason et al. (1986) Science 234:1372-1378).

Expression vectors typically contain a transcription unit or expression cassette that contains all the additional elements required for the expression of the construct or portion(s) thereof, in host cells. A typical expression cassette contains a promoter operably linked to the nucleic acid encoding the construct, such as an antibody fragment, domain, derivative or homolog thereof, or other polypeptide as described herein (e.g., TNF muteins and fusion proteins), and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence, or can be obtained from different genes. For example, a vectors include, a promoter operably linked to nucleic acids encoding a desired polypeptide, or a domain, fragment, derivative or homolog hereof, one or more origins of replication, and optionally, one or more selectable markers (e.g., an antibiotic resistance gene).

Expression systems can have markers that provide gene amplification, such as thymidine kinase and dihydrofolate reductase. Expression systems not involving gene amplification are also suitable, such as using a baculovirus vector in insect cells, with a nucleic acid sequence encoding a polypeptide under the direction of the polyhedron promoter or other strong baculovirus promoter.

For purposes herein, vectors are provided that contain a sequence of nucleotides that encode the Fc region of an IgG antibody, generally a modified Fc, operably linked to the nucleic acid encoding a TNFR1 antagonist or TNFR2 agonist polypeptide, and encoding a linker in between, such as an IgG hinge sequence and/or a short peptide linker, such as a GS linker, including glycine rich flexible linkers, such as (Gly₄Ser)ₙ, where n is a positive integer, such as 1-5 or more, and others of the linkers as described herein or known to those of skill in the art. The vector can include the sequence for one or all of a C_{H}1, C_{H}2, hinge, C_{H}3 or C_{H}4, and/or C_{L}. Generally, such as for expression of Fabs, the vector contains the sequence for a C_{H}1 or C_{L} (kappa or lambda light chains). For example, V_{H}-C_{H}1 and V_{L}-C_{L} sequences can be inserted into a suitable expression vector for the expression of Fab molecules. The sequences of constant regions or hinge regions are known to one of skill in the art (see. e.g., U.S. Publication No. 2008/0248028). Examples of such sequences are provided herein.

Typically, vectors can be plasmids, viral vectors, or others known in the art, used for expression of the polypeptides in vivo or in vitro. For example, the constructs provided herein, such nucleic acid encoding TNFR1 antagonist and TNFR2 agonist polypeptide constructs, are expressed in mammalian cells, including, for example, Chinese Hamster Ovary (CHO) cells.

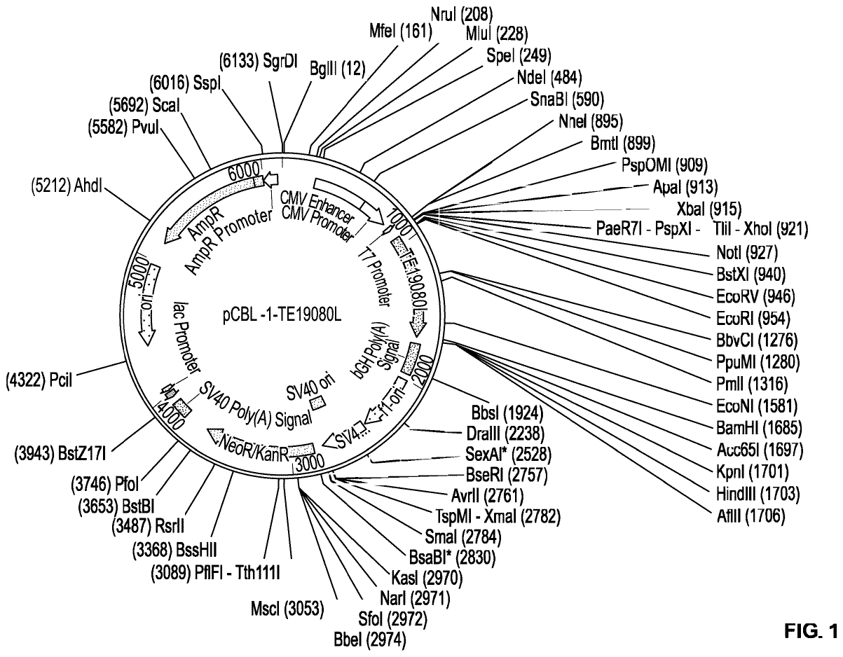

Exemplary eukaryotic vectors include, for example, well known readily available vectors, such as pCMV (Agilent Technologies), pCDNA3.1 (Invitrogen (Thermo Fisher Scientific)), pCBL (from Creative BioLabs, see, e.g., FIG. 1). Other eukaryotic vectors, for example any containing regulatory elements from eukaryotic viruses, can be used as eukaryotic expression vectors. These include, for example, SV40 vectors, papilloma virus vectors, and vectors derived from Epstein-Bar virus. Exemplary eukaryotic vectors include, for example, pMSG, pAV009/A+, pMT010/A+, pMAMneo-5, baculovirus pDSCE, and any other vector allowing for the expression of proteins under the direction of the CMV promoter, SV40 early promoter, SV40 late promoter, metallothionein promoter, murine mammary tumor virus promoter, Rous sarcoma virus promoter, polyhedron promoter, or other promoters shown to be effective for expression in eukaryotes.

Viral vectors, such as adenovirus, retrovirus or vaccinia virus vectors, can be employed. In some examples, the vector is a defective or attenuated retroviral or other viral vector (see, e.g., U.S. Pat. No. 4,980,286). For example, a retroviral vector can be used (see, e.g., Miller et al. (1993) Meth. Enzymol. 217:581-599). These retroviral vectors have been modified to delete retroviral sequences that are not necessary for packaging of the viral genome and integration into host cell DNA. In some examples, viruses armed with a nucleic acid encoding a polypeptide herein can facilitate their replication and spread within a target tissue. The virus also can be a lytic virus or a non-lytic virus where the virus selectively replicates under a tissue specific promoter. As the viruses replicate, the co-expression of the polypeptide with viral genes will facilitate the spread of the virus in vivo.

For bacterial expression, vectors include the well-known and widely disseminated vectors pBR322, pUC, pSKF, pET23D, and fusion vectors, such as MBP (Sigma-Aldrich), GST (Sigma-Aldrich) and vectors containing LacZ. Exemplary plasmid vectors for transformation of E. coli cells, include, for example, the pQE expression vectors (available from Qiagen®, Valencia, CA; see also literature published by Qiagen® describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, to and T1 transcriptional terminators, a ColE1 origin of replication, and a beta-lactamase gene for conferring ampicillin resistance. The pQE vectors permit placement of a 6×His tag at either the N- or C-terminus of the recombinant protein. Such plasmids include pQE 32, pQE 30, and pQE 31, which provide multiple cloning sites for all three reading frames and provide for the expression of N-terminally 6×His-tagged proteins. Other exemplary plasmid vectors for transformation of E. coli cells, include, for example, the pET expression vectors (see, e.g., U.S. Pat. No. 4,952,496; available from NOVAGEN, Madison, WI; see, also literature published by NOVAGEN describing the system). Such plasmids include pET 11a, which contains the T71ac promoter, the T7 terminator, the inducible E. coli lac operator, and the lac repressor gene; pET 12a-c, which contains the T7 promoter, the T7 terminator, and the E. coli ompT secretion signal; and pET 15b and pET19b (NOVAGEN, Madison, WI), which contain a His-Tag™ leader sequence for use in purification with a His column, and a thrombin cleavage site that permits cleavage following purification over the column, the T7-lac promoter region and the T7 terminator.

Cells containing the vectors also are provided. Generally, any cell type that can be engineered to express heterologous DNA and has a secretory pathway is suitable. The cells include eukaryotic and prokaryotic cells, and the vectors are any suitable for use therein. Generally, the cell is a cell that is capable of effecting glycosylation of the encoded protein. Prokaryotic and eukaryotic cells containing the vectors are provided. Such cells include bacterial cells, yeast cells, fungal cells, Archea, plant cells, insect cells and animal, particularly mammalian, cells. The cells are used to produce a polypeptide by growing the above-described cells under conditions whereby the encoded polypeptide is expressed by the cell, and recovering the expressed polypeptide. For purposes herein, for example, the polypeptide can be secreted into the medium.

A host cell strain can be chosen for its ability to modulate the expression of the inserted sequences or to process the expressed protein in the desired fashion. Such modifications of the polypeptide include, but are not limited to, acetylation, carboxylation, glycosylation, phosphorylation, lipidation and acylation. Post-translational processing can impact the folding and/or function of the polypeptide. Different host cells, such as, but not limited to, Chinese hamster ovary (CHO) cells, such as DG44, FreeStyle™ CHO-S cells (Invitrogen), DXB11, CHO-K1), HeLa, MCDK, HEK293 and WI38 cells, have specific cellular machinery and characteristic mechanisms for such post-translational activities, and can be chosen to ensure the correct modification and processing of the introduced protein. Generally, the choice of cell is one that is capable of introducing N-linked glycosylation into the expressed polypeptide. Hence, eukaryotic cells containing the vectors are provided. Exemplary of eukaryotic cells are mammalian Chinese Hamster Ovary (CHO) cells. For example, CHO cells deficient in dihydrofolate reductase (DHFR-), such as DG44 cells, are used to produce polypeptides provided herein.

### 4. Expression

The polypeptide constructs provided herein, including TNFR1 antagonist constructs, TNFR2 agonist constructs, TNFR1 antagonist/TNFR2 agonist multi-specific constructs, and portions thereof, can be produced by any method known to those of skill in the art for protein production, including in vivo and in vitro methods, recombinant and synthetic and chemical methods. Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, known to those of skill in the art; these include regulatory and safety considerations, production costs and the need and methods for purification. Purification methods, and methods for assembly of components, are well known to those of skill in the art.

Expression in eukaryotic hosts can include expression in yeasts, such as Saccharomyces cerevisiae and Pichia pastoris, insect cells, such as Drosophila cells and lepidopteran cells, plants and plant cells, such as tobacco, corn, rice, algae, and lemna. Eukaryotic cells for expression also include mammalian cells lines, such as Chinese hamster ovary (CHO) cells, human embryonic kidney (HEK293) cells, or baby hamster kidney (BHK) cells. Eukaryotic expression hosts also include production in transgenic animals, for example, including production in serum, milk and eggs.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vectors in the cells.

The TNFR1 antagonists, TNFR2 agonists and bi-specific TNFR1 antagonist/TNFR2 agonist constructs herein also can be expressed as protein fusions. For example, a fusion protein can be generated to add additional functionality to a polypeptide. Examples of fusion proteins include, but are not limited to, fusions of a signal sequence, a tag such as for localization, e.g., a his₆ tag or a myc tag, or a tag for purification, for example, a GST fusion, and a sequence for directing protein secretion and/or membrane association. Fusion proteins also include fusions with an Fc region of an IgG, and a linker, such as a hinge sequence of an IgG, and/or a glycine-serine (GS) peptide linker. Alternatively, in some embodiments, the TNFR1 antagonists, TNFR2 agonists and bi-specific TNFR1 antagonist/TNFR2 agonist constructs herein also can fused to serum albumin.

For long-term, high-yield production of recombinant proteins, stable expression is desired. For example, cell lines that stably express a polypeptide can be transformed using expression vectors that contain viral origins of replication or endogenous expression elements and a selectable marker gene. Following the introduction of the vector, cells can be allowed to grow for 1-2 days in an enriched media before they are switched to selective media. The purpose of the selectable marker is to confer resistance to selection, and its presence allows growth and recovery of cells that successfully express the introduced sequences. Resistant cells of stably transformed cells can be proliferated using tissue culture techniques appropriate to the cell types.

Any number of selection systems can be used to recover transformed cell lines. These include, but are not limited to, the herpes simplex virus (HSV) thymidine kinase (TK) (see, e.g., Wigler et al., (1977) Cell 11:223-232) and adenine phosphoribosyltransferase (APRT) (see, e.g., Lowy, I. et al. (1980) Cell, 22:817-23) genes, which can be employed in TK⁻ or APRT⁻ cells, respectively. Also, antimetabolite, antibiotic or herbicide resistance can be used as the basis for selection. For example, dihydrofolate reductase (DHFR), which confers resistance to methotrexate (see, e.g., Wigler et al. (1980) Proc. Natl. Acad. Sci. U.S.A. 77:3567-70); npt, which confers resistance to the aminoglycosides neomycin and G-418 (see, e.g., Colbere-Garapin et al. (1981) J. Mol. Biol. 150:1-14); and als or pat, which confer resistance to chlorsulfuron and phosphinothricin acetyltransferase, respectively, can be used. Additional selectable genes have been described, for example, trpB, which allows cells to utilize indole in place of tryptophan, or hisD, which allows cells to utilize histinol in place of histidine (see, e.g., Hartman, S. C. and R. C. Mulligan (1988) Proc. Natl. Acad. Sci. U.S.A. 85:8047-8051). Visible markers, such as, but not limited to, anthocyanins, beta glucuronidase and its substrate, GUS, and luciferase and its substrate luciferin, also can be used to identify transformants and also to quantify the amount of transient or stable protein expression attributable to a particular vector system (see, e.g., Rhodes et al. (1995) Methods Mol. Biol. 55:121-131).

a. Prokaryotic Cells

Prokaryotes, especially E. coli, provide a system for producing large amounts of proteins. Prokaryotic expression systems generally are used for production of products that are not glycosylated. Transformation of E. coli protocols are well-known to those of skill in the art. Expression vectors for E. coli can contain inducible promoters; such promoters are useful for inducing high levels of protein expression and for expressing proteins that exhibit some toxicity to the host cells. Examples of inducible promoters include, for example, the lac promoter, the trp promoter, the hybrid tac promoter, the T7 and SP6 RNA promoters, and the temperature regulated λPL promoter.

Polypeptides and fusion proteins constructs provided herein, such as any provided herein, can be expressed in the cytoplasmic environment of E. coli. The cytoplasm is a reducing environment, and for some molecules, this can result in the formation of insoluble inclusion bodies. Reducing agents, such as dithiothreitol and 3-mercaptoethanol, and denaturants, such as guanidine-HCl and urea, can be used to resolubilize the proteins. An alternative approach is the expression of proteins in the periplasmic space of bacteria which provides an oxidizing environment and chaperonin-like and disulfide isomerases, and can lead to the production of soluble protein. Typically, a leader sequence is fused to the protein to be expressed, which directs the protein to the periplasm. The leader is then removed by signal peptidases inside the periplasm. Exemplary pathways to translocate expressed proteins into the periplasm are the Sec pathway, the SRP pathway and the TAT pathway. Examples of periplasmic-targeting leader sequences include the pelB leader from the pectate lyase gene, the StII leader sequence, and the DsbA leader sequence, and the leader derived from the alkaline phosphatase gene. In some cases, periplasmic expression allows leakage of the expressed protein into the culture medium. The secretion of proteins allows for quick and simple purification from the culture supernatant. Proteins that are not secreted can be obtained from the periplasm by osmotic lysis. Similar to cytoplasmic expression, in some cases, proteins can become insoluble, and denaturants and reducing agents can be used to facilitate solubilization and refolding. The temperature of induction and growth also can influence expression levels and solubility; typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts, such as Saccharomyces cerevisiae, Schizosaccharomyces pombe, Yarrowia lipolytica, Kluyveromyces lactis, and Pichia pastoris, are well-known expression hosts that can be used for the production of proteins, such as any described herein. Yeast can be transformed with episomal replicating vectors or by stable chromosomal integration by homologous recombination. Typically, inducible promoters are used to regulate gene expression. Examples of such promoters include GAL1, GAL7, and GALS, and metallothionein promoters, such as CUP1, AOX1 or other Pichia or other yeast promoters. Expression vectors often include a selectable marker such as LEU2, TRP1, HIS3 and URA3, for selection and maintenance of the transformed DNA. Proteins expressed in yeast are often soluble. Co-expression with chaperonins, such as Bip and protein disulfide isomerase, can improve expression levels and solubility. Additionally, proteins expressed in yeast can be directed for secretion using secretion signal peptide fusions, such as the yeast mating type alpha-factor secretion signal from Saccharomyces cerevisiae, and fusions with yeast cell surface proteins, such as the Aga2p mating adhesion receptor, or the Arxula adeninivorans glucoamylase. A protease cleavage site, such as for the Kex-2 protease, can be engineered to remove the fused sequences from the expressed polypeptides as they exit the secretion pathway. Yeast also is capable of glycosylation at Asn-X-Ser/Thr motifs.

c. Insects and Insect Cells

Insect cells, particularly using baculovirus expression, are useful for expressing polypeptides, including antibodies or fragments thereof. Insect cells express high levels of protein and are capable of most of the post-translational modifications used by higher eukaryotes. Baculovirus have a restrictive host range which improves the safety and reduces regulatory concerns of eukaryotic expression. Typically, expression vectors use a promoter for high level expression, such as the polyhedrin promoter and p10 promoter of baculovirus. Baculovirus systems include baculoviruses, such as Autographa californica nuclear polyhedrosis virus (AcNPV), and the Bombyx mori nuclear polyhedrosis virus (BmNPV), and an insect cell line, such as Sf9 derived from Spodoptera frugiperda, TN derived from Trichoplusia ni, A7S derived from Pseudaletia unipuncta, and DpN1 derived from Danaus plexippus. For high-level expression, the nucleotide sequence of the molecule to be expressed is fused immediately downstream of the polyhedrin initiation codon of the virus. To generate baculovirus recombinants capable of expressing human antibodies, a dual-expression transfer, such as pAcUW51 (PharMingen) can be used. Mammalian secretion signals are accurately processed in insect cells and can be used to secrete the expressed protein into the culture medium. The cell lines Pseudaletia unipuncta (A7S) and Danaus plexippus (DpN1) produce proteins with glycosylation patterns similar to mammalian cell systems. Exemplary insect cells are those that have been altered to reduce immunogenicity, including those with “mammalianized” baculovirus expression vectors and those lacking the enzyme FT3.

An alternative expression system in insect cells is the use of stably transformed cells. Cell lines, such as the Schneider 2 (S2) and Kc cells (Drosophila melanogaster), and C7 cells (Aedes albopictus), can be used for expression. The Drosophila metallothionein promoter can be used to induce high levels of expression in the presence of heavy metal induction with cadmium or copper. The baculovirus immediate early gene promoter IE1 can be used to induce consistent levels of expression. Typical expression vectors include the pIE1-3 and pI31-4 transfer vectors (Novagen). Expression vectors are typically maintained by the use of selectable markers, such as neomycin and hygromycin.

d. Mammalian Expression Cells

Mammalian expression systems can be used to express polypeptides, including the constructs herein, including TNFR1 antagonists, TNFR2 agonists, bi-specific TNFR1 antagonist/TNFR2 agonist constructs, and fusions thereof, provided herein. Expression constructs can be transferred to mammalian cells by viral infection, such as with adenovirus, or by direct DNA transfer, such as by using liposomes, calcium phosphate and DEAE-dextran, and by physical means, such as electroporation and microinjection. Expression vectors for mammalian cells typically include an mRNA cap site, a TATA box, a translational initiation sequence (Kozak consensus sequence) and polyadenylation elements. Internal ribosomal entry site (IRES) elements also can be added to permit bicistronic expression with another gene, such as a selectable marker. Such vectors often include transcriptional promoter-enhancers for high-level expression, such as, for example, the SV40 promoter-enhancer, the human cytomegalovirus (CMV) promoter, and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha-fetoprotein, alpha-1 antitrypsin, beta-globin, myelin basic protein, myosin light chain-2, and gonadotropic releasing hormone gene control.

Selectable markers can be used to select for and maintain cells with the expression construct. Examples of selectable marker genes include, but are not limited to, hygromycin B phosphotransferase, adenosine deaminase, xanthine-guanine phosphoribosyltransferase, aminoglycoside phosphotransferase, dihydrofolate reductase (DHFR), and thymidine kinase (TK). For example, expression can be performed in the presence of methotrexate to select for only those cells expressing the DHFR gene. Modified anti-TNFR antibodies and antigen-binding fragments thereof can be produced, for example, using a NEO^{R}/G418 system, a dihydrofolate reductase (DHFR) system or a glutamine synthetase (GS) system. The GS system uses joint expression vectors, such as pEE12/pEE6, to express both heavy chain and light chain. Fusion with cell surface signaling molecules, such as TCR-ζ and Fc_{ε}RI-γ, can direct expression of the proteins in an active state on the cell surface.

Many cell lines are available for mammalian expression, including mouse, rat, human, monkey, chicken and hamster cells. Exemplary cell lines include, but are not limited to, BHK (e.g., BHK-21 cells), 293-F, CHO, CHO Express (CHOX; ExcellGene), Balb/3T3, HeLa, MT2, mouse NSO (non-secreting), and other myeloma cell lines, hybridoma and heterohybridoma cell lines, lymphocytes, fibroblasts, Sp2/0, COS, NIH3T3, HEK293, 293S, 2B8, and HKB cells. Cell lines that are adapted to serum-free media also are available, which facilitates purification of secreted proteins from the cell culture media. Examples include CHO-S cells (Invitrogen®, Carlsbad, CA, cat #11619-012) and the serum free EBNA-1 cell line (see, e.g., Pham et al. (2003) Biotechnol. Bioeng. 84:332-342). Cell lines that are adapted for growth in special mediums that are optimized for maximal expression also are available. For example, DG44 CHO cells are adapted to grow in suspension culture in a chemically defined, animal product-free medium.

e. Plants

Transgenic plant cells and plants can be used to express polypeptides and proteins, such as any described herein. Expression constructs are typically transferred to plants using direct DNA transfer, such as by microprojectile bombardment and PEG-mediated transfer into protoplasts, and with agrobacterium-mediated transformation. Expression vectors can include promoter and enhancer sequences, transcriptional termination elements, and translational control elements. Expression vectors and transformation techniques are usually divided between dicot hosts, such as Arabidopsis and tobacco, and monocot hosts, such as corn and rice. Examples of plant promoters used for expression include, for example, the cauliflower mosaic virus promoter (CaMV 35S), the nopaline synthase promoter, the ribose bisphosphate carboxylase promoter, and the ubiquitin (e.g., maize ubiquitin-1 (ubi-1)) and UBQ3 promoters. Selectable markers, such as hygromycin, phosphomannose isomerase and neomycin phosphotransferase, are often used to facilitate selection and maintenance of transformed cells. Transformed plant cells can be maintained in culture as cells, aggregates (callus tissue) or regenerated into whole plants. Transgenic plant cells also can include algae engineered to produce polypeptides. Because plants have different glycosylation patterns than mammalian cells, this can influence the choice of protein produced in these hosts.

### 5. Purification

Host cells transformed with a nucleic acid encoding polypeptide constructs provided herein can be cultured under conditions suitable for the expression and recovery of the encoded protein from cell culture. The protein produced by a recombinant cell is generally secreted, but can be contained intracellularly, depending on the sequence and/or the vector used. As understood by those of skill in the art, expression vectors containing nucleic acid molecules encoding polypeptides provided herein can be designed with signal sequences that facilitate direct secretion of expressed polypeptides through prokaryotic or eukaryotic cell membranes.

Methods for purification of polypeptides from host cells depend on the chosen host cells and expression systems. For secreted molecules, proteins are generally purified from the culture media after removing the cells. For intracellular expression, cells can be lysed, and the proteins can be purified from the extract. When transgenic organisms, such as transgenic plants and animals, are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art.

Polypeptides, such as the TNFR1 antagonist constructs, TNFR2 agonist constructs, TNFR1 antagonist/TNFR2 agonist bi-specific constructs, and other constructs provided herein, and components thereof, can be purified using protein purification techniques known to those of skill in the art. These include, but are not limited to, limited to, SDS-PAGE, size fractionation and size exclusion chromatography, ammonium sulfate precipitation, chelate chromatography, column chromatography, HPLC, dialysis, and ionic exchange chromatography, such as anion exchange, and combinations thereof. Affinity purification techniques also can be used. The constructs herein can be purified using methods developed for purification of antibodies and antibody fragments. Exemplary of a method to purify antibodies and antibody fragments are methods that include column chromatography, where a solid support column material is linked to Protein G, a cell surface-associated protein from Streptococcus, that binds immunoglobulins with high affinity. Antibodies and antibody fragments also can be purified by methods that include protein A chromatography, in which protein A, a cell surface-associated protein from Staphylococcus aureus, which binds immunoglobulins, such as IgGs, with high affinity, is bound to a solid support column. Other immunoglobulin-binding bacterial proteins that can be used to purify the antibodies and antibody fragments, include Protein A/G, a recombinant fusion protein that combines the IgG binding domains of Protein A and Protein G; and Protein L, a surface protein from Peptostreptococcus (see, e.g., Bjorck (1988) J. Immunol. 140(4):1194-1197; Kastern et al. (1992) J. Biol. Chem. 267(18):12820-12825; Eliasson et al. (1988) J. Biol. Chem. 263:4323-4327). The constructs are substantially pure, which is typically at least or at least about 90%, 91%, 92%, 93%, 94%, 95%, 96%, 97%, 98% or 99% purity. Purity can be assessed by standard methods, such as by SDS-PAGE and Coomassie blue staining.

When antibodies and fragments thereof and related polypeptides are expressed by transformed bacteria in large amounts, typically after promoter induction, although expression can be constitutive, the polypeptides can form insoluble aggregates. Protocols for purification of polypeptide inclusion bodies are known to one of skill in the art. For example, in one method, a cell suspension is centrifuged to pellet the inclusion bodies, and the pellet containing the inclusion bodies is re-suspended in a buffer, such as, for example, 20 mM Tris-HCL (pH 7.2), 1 mM EDTA, 150 mM NaCl and 2% Triton-X 100, a non-ionic detergent, which does not harm the inclusion bodies, but dissolved contaminants. The wash step can be repeated to remove as much cellular debris as possible. The remaining pellet of inclusion bodies is re-suspended in an appropriate buffer, such as 20 mM sodium phosphate, pH 6.8, 150 mM NaCl. Other appropriate buffers, as well as alternative purification protocols, are known or can be developed by one of skill in the art.

Other methods for purifying, antibodies and fragments thereof, and the constructs provided herein, can be used or developed. They can be purified from bacterial periplasm. For polypeptides exported into the periplasm of bacteria in which the polypeptide is produced, the periplasmic fraction of the bacteria can be isolated by cold osmotic shock, in addition to other methods known to those of skill in the art. For example, in one method, to isolate recombinant polypeptides from the periplasm, the bacterial cells are centrifuged to form a pellet. The pellet is re-suspended in a buffer containing 20% sucrose. To lyse the cells, the bacteria are centrifuged and the pellet is re-suspended in ice-cold 5 mM MgSO₄ and kept in an ice bath for approximately 10 minutes. The cell suspension is centrifuged and the supernatant is decanted and saved. The recombinant polypeptides present in the supernatant can be separated from the host proteins by standard separation techniques that are well-known to those of skill in the art. These methods include, but are not limited to, the following steps: solubility fractionation, size differential filtration, and column chromatography.

Expression constructs also can be engineered to include a nucleic acid encoding an affinity tag, for example for detection or purification of the expressed product, operatively linked, upon expression to the encoded polypeptide. Affinity tags, include, for example, a Small Ubiquitin-like Modifier (SUMO) tag, a myc epitope, a GST fusion or His₆, for affinity purification with SUMO, myc antibody, glutathione resin and Ni-resin, respectively. Nucleic acid encoding such tags can be joined to the nucleic acid encoding the polypeptide constructs provided herein. The tags can facilitate purification and/or detection of soluble proteins. For example, a TNFR1 antagonist polypeptide construct or portion thereof can be expressed as a recombinant protein with one or more additional polypeptide domains added to facilitate protein purification. Purification facilitating domains include, but are not limited to, metal chelating peptides, such as histidine-tryptophan modules that allow purification on immobilized metals, protein A domains that allow purification on immobilized immunoglobulin, and the domain utilized in the FLAGS extension/affinity purification system (Immunex Corp., Seattle, WA). The inclusion of nucleic acid encoding a cleavable linker sequence, such as a Factor Xa cleavable recognize site (Ile-Glu-Gly-Arg, engineered to include a Nru I restrictions site, see, e.g., EP 92115607A) or enterokinase (Invitrogen (Thermo Fisher Scientific), San Diego, CA), between the purification domain and an encoded expressed polypeptide, facilitate purification. An exemplary expression vector encodes for expression of a fusion protein containing a TNFR1 antagonist and/or a TNFR2 agonist polypeptide and an enterokinase cleavage site. The Small Ubiquitin-like Modifier (SUMO) tag facilitates purification on immobilized metal ion affinity chromatography (IMIAC), and the enterokinase cleavage site provides a cleavage site for purifying the polypeptide from the fusion protein.

Purity can be assessed by any method known in the art, including gel electrophoresis, orthogonal HPLC methods, staining and spectrophotometric techniques. The expressed and purified polypeptide can be analyzed using any assay or method known to one of skill in the art, for example, any described herein. These include assays based on the physical and/or functional properties of the polypeptide, including, but not limited to, analysis by gel electrophoresis, immunoassays, binding assays, and assays of TNF-mediated TNFR1 and/or TNFR2 activity.

### 6. Additional Modifications

The modified TNFR1 antagonist constructs, TNFR2 agonist constructs, bi-specific TNFR1 antagonist/TNFR2 agonist constructs, and other constructs, and components thereof provided, as described above, include components (activity modifiers) that alter pharmacological properties, including pharmacokinetic and pharmacodynamic properties. The portions of the constructs, such as TNFR1 inhibitor polypeptides and small molecules, and TNFR2 agonist polypeptides and small molecules, can be conjugated to a polymer or polymeric moiety, such as, but not limited to, a polyethylene glycol (PEG) moiety or dextran, or to human serum albumin (HSA), or can be sialylated to reduce immunogenicity and/or to increase half-life in serum and other body fluids. The polypeptides also can be linked to a purification tag, such as a His tag or a SUMO sequence. Additional modifications include, for example, glycosylation, carboxylation, hydroxylation, phosphorylation, or other known modifications. Glycosylation can be incorporated in vivo, using an appropriate expression system, such as a mammalian expression system, in vitro, or via a combination of in vivo and in vitro methods in which, for example, the polypeptide is expressed in prokaryotic cells and is further modified in vitro using enzymatic transglycosylation. Other modifications can be made in vitro or, for example, by producing the modified polypeptide in a suitable host that produces such modifications.

These modifications or activity modifiers and modifications are effected and selected so that the modified polypeptide incorporates the functionality of the modification and retains at least a portion, generally at least 50%, 60%, 70%, 80%, 90%, or 95% activity compared with a non-fused or unmodified polypeptide, including 96%, 97%, 98%, 99% or greater activity compared with a non-fusion or unmodified polypeptide. For example, TNFR1 antagonist constructs retain the ability to inhibit TNF-mediated signaling via TNFR1.

Linkage of a polypeptide such as a TNFR1 antagonist or TNFR2 agonist, with another polypeptide, can be effected directly or indirectly via a linker. In one example, linkage can be by chemical linkage, such as via heterobifunctional agents, or thiol linkages, or other such linkages. Fusion also can be effected by recombinant expression. Fusion of a polypeptide, such as a TNFR1 antagonist or TNFR2 agonist, to another polypeptide, can be to the N- or C-terminus of the TNFR1 antagonist or TNFR2 agonist polypeptide. Non-limiting examples of polypeptides that can be used in fusion proteins with a TNFR1 antagonist or TNFR2 agonist polypeptide provided herein include, for example, a GST (glutathione S-transferase) polypeptide, an Fc domain from immunoglobulin G, albumin, a heterologous signal sequence, and combinations thereof.

The encoded constructs can be produced by standard recombinant techniques. For example, DNA fragments encoding the different polypeptide portions can be ligated together in-frame, in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene can be synthesized by conventional techniques, including automated DNA synthesizers. Alternatively, PCR amplification of gene fragments can be carried out using anchor primers that give rise to complementary overhangs between two consecutive gene fragments that subsequently can be annealed and re-amplified to generate a chimeric gene. Many expression vectors are commercially available that already encode a fusion moiety (e.g., a GST polypeptide). A polypeptide-encoding nucleic acid can be cloned into such an expression vector such that the fusion moiety is linked in-frame to the TNFR1 antagonist, TNFR2 agonist, and multi-specific, such as bi-specific, constructs provided herein.

a. PEGylation

Polyethylene glycol (PEG) is used in biomaterials, biotechnology and medicine; it is a biocompatible, nontoxic, water-soluble polymer that generally is non-immunogenic. In the area of drug delivery, PEG derivatives are used in covalent attachment to proteins (i.e., “PEGylation”), to reduce immunogenicity, proteolysis, and kidney clearance, and to increase serum half-life, and enhance solubility (see, e.g., Zalipsky (1995) Adv. Drug Del. Rev. 16:157-182). PEG has been attached to low molecular weight, relatively hydrophobic drugs to enhance solubility, reduce toxicity and alter biodistribution. Conjugation to linear or branched-chain PEG moieties increases the molecular mass and hydrodynamic radius of the polypeptide, and decreases the rate of glomerular filtration by the kidneys. Typically, PEGylated drugs are administered, such as by injection, as solutions. In the constructs herein, PEGylation moieties and other such polymers can be part of the linker portions of the constructs.

A related application is the synthesis of cross-linked degradable PEG networks or formulations for use in drug delivery, since much of the same chemistry used in the design of degradable, soluble drug carriers also can be used in the design of degradable gels (see, e.g., Sawhney et al. (1993) Macromolecules 26:581-587). Intermacromolecular complexes can be formed by mixing solutions of two complementary polymers. Such complexes are stabilized by electrostatic interactions (polyanion-polycation) and/or hydrogen bonds (polyacid-polybase) between the polymers involved, and/or by hydrophobic interactions between the polymers in a medium (see, e.g., Krupers et al. (1996) Eur. Polym. J. 32:785-790). For example, mixing solutions of polyacrylic acid (PAAc) and polyethylene oxide (PEO) under the proper conditions results in the formation of complexes based mostly on hydrogen bonding. Dissociation of these complexes at physiologic conditions has been used for delivery of free (i.e., non-PEGylated) drugs. Complexes of complementary polymers have been formed from homopolymers and copolymers.

Numerous reagents for PEGylation are known, as are PEGylated therapeutic proteins. Such reagents include, but are not limited to, N-hydroxysuccinimidyl (NHS) activated PEG, succinimidyl mPEG, mPEG₂-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG₂ butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see, e.g., Veronese et al. (1997) J. Bioactive Compatible Polymers 12:197-207; and numerous U.S. and worldwide patents). In one example, the polyethylene glycol has a molecular weight ranging from about 3 kDa to about 50 kDa, and typically from about 5 kDa to about 30 kDa. Covalent attachment of the PEG to the drug (known as “PEGylation”) can be accomplished by known chemical synthesis techniques. For example, the PEGylation of protein can be accomplished by reacting NHS-activated PEG with the protein under suitable reaction conditions.

While numerous reactions have been described for PEGylation, those that are most generally applicable for proteins confer directionality, use mild reaction conditions, and do not necessitate extensive downstream processing to remove toxic catalysts or bi-products. For instance, monomethoxy PEG (mPEG) has only one reactive terminal hydroxyl, and thus, its use limits some of the heterogeneity of the resulting PEG-protein product mixture. Activation of the hydroxyl group at the end of the polymer opposite to the terminal methoxy group is generally necessary to accomplish efficient protein PEGylation, with the aim being to make the derivatized PEG more susceptible to nucleophilic attack. The attacking nucleophile is usually the epsilon-amino group of a lysyl residue, but other amines also can react (e.g., the N-terminal alpha-amine or the ring amines of histidine), if local conditions are favorable. A more directed attachment is possible in proteins containing a single lysine or cysteine. The latter residue can be targeted by PEG-maleimide for thiol-specific modification. Alternatively, PEG hydrazide can be reacted with a periodate oxidized protein and reduced in the presence of NaCNBH₃. More specifically, PEGylated CMP sugars can be reacted with a protein in the presence of appropriate glycosyl-transferases. One technique is the “PEGylation” technique where a number of polymeric molecules are coupled to the polypeptide in question. When using this technique, the immune system has difficulties in recognizing the epitopes on the polypeptide's surface that are responsible for the formation of antibodies, thereby reducing the immune response. For polypeptides introduced directly into the circulatory system of the human body to give a particular physiological effect (i.e., pharmaceuticals), the typical potential immune response is an IgG and/or IgM response, while polypeptides which are inhaled through the respiratory system (i.e., industrial polypeptides) potentially can cause an IgE response (i.e., allergic response). One of the theories explaining the reduced immune response is that the polymeric molecule(s) shield(s) epitope(s) on the surface of the polypeptide that are responsible for the immune response leading to antibody formation. Another theory, or at least a partial factor, is that the heavier the conjugate is, the more reduced the immune response.

For example, PEGylate polypeptide constructs and polypeptide components provided herein, PEG moieties are conjugated, via covalent attachment, to the polypeptides. Techniques for PEGylation include, but are not limited to, the use of specialized linkers and coupling chemistries (see, e.g., Harris (2002) Adv. Drug Deliv. Rev. 54:459-476), attachment of multiple PEG moieties to a single conjugation site (such as via use of branched PEGs; see, e.g., Veronese et al., (2002) Bioorg. Med. Chem. Lett. 12:177-180), site-specific PEGylation and/or mono-PEGylation (see, e.g., Chapman et al. (1999) Nature Biotech. 17:780-783), and site-directed enzymatic PEGylation (see, e.g., Sato (2002) Adv. Drug Deliv. Rev. 54:487-504). Methods and techniques described in the art can produce proteins having 1, 2, 3, 4, 5, 6, 7, 8, 9, 10, or more than 10, PEG or PEG derivatives attached to a single protein molecule (see, e.g., U.S. Patent Publication No. 2006/0104968).

b. Albumination

The polypeptides provided herein, such as the TNFR1 antagonist constructs, TNFR2 agonist constructs, and multi-specific TNFR1 antagonist/TNFR2 agonist constructs, can be fused to albumin (i.e., “albuminated”), for human therapeutics, to human serum albumin (HSA), to increase the half-life, stability, bioavailability, and distribution, and/or to improve the pharmacological properties, such as pharmacokinetics, of the polypeptides. Numerous products linked to human serum albumin (HSA) are approved for use as therapeutics, including for use as cancer therapeutics and for the treatment of type 2 diabetes (see, e.g., AlQahtani et al. (2019) Biomed and Pharmacotherapy 113:108750; Roscoe et al. (2018) Mol. Pharmaceutics 151:15046-5047; Strohl, W. R. (2015) BioDrugs 4:215-239). In some examples, the mature HSA protein, lacking the signal sequence and activation sequence, is fused to a protein of interest. In some examples, serum albumin, such as human serum albumin (HSA), is conjugated to the polypeptide. An exemplary HSA protein is set forth in SEQ ID NO:35.

Fusions with HSA are provided herein. These include fusion with HSA to the N- or C-terminus of the TNFR1 antagonist (e.g., dAbs, scFvs, Fabs or other antigen-binding fragments, as provided herein), or the TNFR2 agonists (e.g., TNF mutein), generally, via a short peptide linker, such as, but not limited to, a glycine-serine (GS) linker, such as (GSGS)ₙ or (GGGGS)ₙ, where n=1-5 or 6. Exemplary TNFR1 antagonist-HSA fusions are set forth in SEQ ID NOs: 709, 713, 717, 721, and 725.

c. Purification Tags

In some examples, the TNFR1 antagonist constructs, TNFR2 agonist constructs, multi-specific, such as bi-specific, TNFR1 antagonist/TNFR2 agonist constructs and fusion proteins, provided herein, can contain a tag for purification of the product. Exemplary tags for purification are described elsewhere herein. For example, exemplary polypeptides herein can contain a SUMO or His sequence for purification. Generally, the tags are cleavable tags.

The polypeptide constructs, including fusion proteins, provided herein can include a His purification tag, such as a 6×His tag. His-tagged polypeptides optionally can contain a fusion partner, and/or a signal for expression and secretion. For example, the exemplary His-polypeptide fusion proteins can contain one or more of a human immunoglobulin light chain kappa (x) leader signal peptide sequence (SEQ ID NO:835), a 6×His tag (SEQ ID NO:836), a SUMO sequence (SEQ ID NO:837), and HSA (SEQ ID NO:35). In another example, the exemplary His tagged-polypeptide fusion proteins can contain the human immunoglobulin light chain kappa (x) leader signal peptide sequence (SEQ ID NO:835), a 6×His tag (SEQ ID NO:836), a SUMO sequence (SEQ ID NO:837), and an IgG Fc (see, e.g., SEQ ID NOs: 10, 12, 14, 16, 27, and 30).

In some embodiments, the polypeptides and fusion proteins provided herein can include a His tag and/or SUMO sequences for accumulation in inclusion bodies. For example, the His-SUMO sequence set forth in SEQ ID NO:838, can be linked to any of the polypeptides or fusion proteins provided herein. His-SUMO tagged polypeptides optionally can contain a fusion partner, and/or a signal for expression and secretion. For example, the His-SUMO-polypeptide fusion proteins can contain the human immunoglobulin light chain kappa (κ) leader signal peptide sequence (SEQ ID NO:835), a 6×His tag (SEQ ID NO:836), a SUMO sequence (SEQ ID NO:837), and HSA (SEQ ID NO:35). In another example, the exemplary His-SUMO-polypeptide fusion proteins can contain the human immunoglobulin light chain kappa (x) leader signal peptide sequence (SEQ ID NO:835), a 6×His tag (SEQ ID NO:836), a SUMO sequence (SEQ ID NO:837), and an IgG Fc (see, e.g., SEQ ID NOs: 10, 12, 14, 16, 27, and 30).

### 7. Nucleic Acid Molecules and Gene Therapy

Nucleic acid molecules encoding polypeptide constructs that are fusion proteins that are provided herein can be used for gene therapy, such as, for expression in gene therapy vectors or for administration as DNA or RNA constructs. Among these, some of the TNFR1 antagonist, TNFR2 agonist, and bi-specific TNFR1 antagonist/TNFR2 agonist constructs that are provided herein are provided as fusion proteins. Nucleic acid molecules encoding these constructs, as well as vectors and other delivery vehicles, are provided herein. The nucleic acid molecules can encode polypeptides having at least 80%, 85%, 90%, 91%, 92%, 93%, 94%, 95%, 96%, 97%, 98%, or 99% sequence identity to any polypeptide or construct provided herein. In another embodiment, a nucleic acid molecule can include those with degenerate codon sequences encoding any of the polypeptides or constructs provided herein.

The nucleic acids molecules for use for in gene therapy are operably-linked to regulatory sequences of nucleic acids, as needed. These include promoters, enhancers, signal sequences and other trafficking sequences, and other such regulatory sequences that are well-known to those of skill in the art. For vectors with particular tissue tropisms, the regulatory sequences can be specific for such tissues, such as the liver for long-term gene expression, or the eye for any ophthalmic applications. Exemplary promoters include inducible and constitutive promoters for expression in mammalian cells. Such promoters, which are those recognized in a eukaryotic, such as a mammalian, subject, include, but are not limited to, CMV and SV40 promoters; adenovirus promoters, such as the E2 gene promoter, which is responsive to the E7 oncoprotein; a PV promoter, such as the BPV p89 promoter that is responsive to the PV E2 protein; and other suitable promoters.

A polypeptide provided herein also can be delivered to the cells in gene transfer vectors. The transfer vectors also can encode additional other therapeutic agent(s) for treatment of the disease or disorder, such as rheumatoid arthritis, or any other chronic inflammatory, autoimmune, neurodegenerative, or demyelinating disease or disorder, described herein or known in the art, for which the polypeptide is administered. Transfer vectors encoding a polypeptide provided herein can be used systemically, by administering the nucleic acid molecule to a subject. For example, the transfer vector can be a viral vector, such as an adenovirus vector. Vectors encoding a polypeptide or construct herein also can be incorporated into stem cells, and such stem cells can be administered to a subject, such as by transplanting or engrafting the stem cells at sites for therapy. For example, mesenchymal stem cells (MSCs) can be engineered to express a therapeutic polypeptide, and such MSCs can be engrafted at a transplant site for therapy.

Rather than delivering the protein, the nucleic acid can be administered in vivo, such as systemically, or by any other route, or ex vivo, such as by removal of cells, including lymphocytes, introduction of the nucleic acid therein, and reintroduction into the host, or a compatible recipient.

Polypeptides can be delivered to cells and tissues by expression of nucleic acid molecules. Polypeptides can be administered as nucleic acid molecules encoding the polypeptides, including ex vivo techniques, and direct in vivo expression. Nucleic acids can be delivered to cells and tissues by any method known to those of skill in the art. The isolated nucleic acid sequences can be incorporated into vectors for further manipulation. Methods for administering polypeptides by expression of encoding nucleic acid molecules include administration of recombinant vectors. The vector can be designed to remain episomal, such as by inclusion of an origin of replication, or can be designed to integrate into a chromosome in the cell. Nucleic acid molecules encoding polypeptides provided herein also can be used in ex vivo gene expression therapy using non-viral vectors. For example, cells can be engineered to express a polypeptide, either operatively linked to regulatory sequences, or such that it is placed operatively linked to regulatory sequences in a genomic location. Such cells then can be administered locally or systemically to a subject, such as a patient in need of treatment.

Gene therapy vectors can remain episomal, or can integrate into chromosomes of the treated subject. A polypeptide can be expressed by a virus, which is administered to a subject in need of treatment. Viral vectors suitable for gene therapy include adenoviruses, adeno-associated viruses (AAVs), retroviruses, lentiviruses, vaccinia viruses, and others noted above. Viral vectors, which include, for example, adenoviruses, adeno-associated viruses (AAVs), poxviruses, herpesviruses, retroviruses, and others designed for gene therapy, can be employed. AAV vectors with altered tropism, such as for liver cells, are available. AAV vectors are composed of a capsid that confers the tropism, and nucleic acid encoding the polypeptide flanked by ITRs.

For example, adenovirus expression technology is well-known in the art, and adenovirus production and administration methods also are well-known. Adenovirus serotypes are available, for example, from the American Type Culture Collection (ATCC, Rockville, MD). Adenovirus vectors can be used ex vivo, for example, cells are isolated from a patient in need of treatment, and transduced with a polypeptide-expressing adenovirus vector. After a suitable culturing period, the transduced cells are administered to a subject, locally and/or systemically. Alternatively, polypeptide adenovirus particles are isolated and formulated in a pharmaceutically-acceptable carrier for delivery of a therapeutically effective amount to prevent, treat, or ameliorate a disease or condition of a subject. Typically, adenovirus particles are delivered at a dose ranging from 1 particle to 10¹⁴ particles per kilogram subject weight, generally between 10⁶ or 10⁸ particles to 10¹² particles per kilogram subject weight. In some situations, it is desirable to provide a nucleic acid source with an agent that targets cells, such as an antibody specific for a cell surface membrane protein or a target cell, or a ligand for a receptor on a target cell. The polypeptides or constructs provided herein also can be targeted for delivery into specific cell types. For example, adenoviral vectors encoding the polypeptides or constructs provided herein can be used for stable expression in nondividing cells, such as liver cells (see, e.g., Margaritis et al. (2004) J. Clin. Invest. 113:1025-1031). In another example, viral or non-viral vectors encoding polypeptides or constructs herein can be transduced into isolated cells for subsequent delivery. Additional cell types for expression and delivery include, but are not limited to, fibroblasts and endothelial cells.

The nucleic acid molecules can be introduced into artificial chromosomes, and other non-viral vectors. Artificial chromosomes, such as ACES (see, Lindenbaum et al., (2004) Nucleic Acids Res. 32(21):e172), can be engineered to encode and express the isoform. Briefly, mammalian artificial chromosomes (MACs) provide a means to introduce large payloads of genetic information into the cell in an autonomously replicating, non-integrating format. Unique among MACs, the mammalian satellite DNA-based Artificial Chromosome Expression (ACE) can be reproducibly generated de novo in cell lines of different species and readily purified from the host cells' chromosomes. Purified mammalian ACEs can then be re-introduced into a variety of recipient cell lines where they have been stably maintained for extended periods in the absence of selective pressure using an ACE System. Using this approach, specific loading of one or two gene targets has been achieved in LMTK(−) and CHO cells.

Another method for introducing nucleic acids encoding a polypeptide is a two-step gene replacement technique in yeast, starting with a complete adenovirus genome (Ad2; Ketner et al. (1994) Proc. Natl. Acad. Sci. U.S.A. 91: 6186-6190) cloned in a Yeast Artificial Chromosome (YAC) and a plasmid containing adenovirus sequences to target a specific region in the YAC clone, an expression cassette for the gene of interest and a positive and negative selectable marker. YACs are of particular interest because they permit incorporation of larger genes. This approach can be used for construction of adenovirus-based vectors bearing nucleic acids encoding any of the described polypeptides or constructs herein for gene transfer to mammalian cells or whole animals.

The nucleic acids can be encapsulated in a vehicle, such as a liposome, or introduced into a cell, such as a bacterial cell, particularly an attenuated bacterium, or introduced into a viral vector. For example, when liposomes are employed, proteins that bind to a cell surface membrane protein associated with endocytosis can be used for targeting and/or to facilitate uptake, e.g., capsid proteins or fragments thereof tropic for a particular cell type, antibodies for proteins which undergo internalization in cycling, and proteins that target intracellular localization and enhance intracellular half-life.

For ex vivo and in vivo methods, nucleic acid molecules encoding the polypeptide or construct herein is introduced into cells that are from a suitable donor or the subject to be treated. Cells into which a nucleic acid can be introduced for purposes of therapy include, for example, any desired, available cell type appropriate for the disease or condition to be treated, including but not limited to epithelial cells; endothelial cells; keratinocytes; fibroblasts; muscle cells; hepatocytes; blood cells, such as T lymphocytes, B lymphocytes, monocytes, macrophages, neutrophils, eosinophils, megakaryocytes, and granulocytes; and various stem or progenitor cells, in particular, hematopoietic stem or progenitor cells, such as stem cells obtained from bone marrow, umbilical cord blood, peripheral blood, fetal liver, and other sources thereof.

For ex vivo treatment, cells from a donor compatible with the subject to be treated or cells from the subject to be treated are removed, the nucleic acid is introduced into these isolated cells, and the modified cells are administered to the subject. Treatment includes direct administration, such as, for example, encapsulated within porous membranes, which are implanted into the patient (see, e.g., U.S. Pat. Nos. 4,892,538 and 5,283,187, each of which is herein incorporated by reference in its entirety). Techniques suitable for the transfer of nucleic acid into mammalian cells in vitro include the use of liposomes and cationic lipids (e.g., DOTMA, DOPE and DC-Chol) electroporation, microinjection, cell fusion, DEAE-dextran, and calcium phosphate precipitation methods. Methods of DNA delivery can be used to express the polypeptides or constructs provided herein in vivo. Such methods include liposome delivery of nucleic acids and naked DNA delivery, including local and systemic delivery, such as using electroporation, ultrasound, and calcium-phosphate delivery. Other techniques include microinjection, cell fusion, chromosome-mediated gene transfer, microcell-mediated gene transfer, and spheroplast fusion.

In vivo expression of a polypeptide or construct herein can be linked to expression of additional molecules. For example, expression of a polypeptide can be linked with expression of a cytotoxic product, such as in an engineered virus, or expressed in a cytotoxic virus. Such viruses can be targeted to a particular cell type that is a target for a therapeutic effect.

In vivo expression of a polypeptide or construct provided herein can include operatively linking polypeptide-encoding nucleic acid molecules to specific regulatory sequences, such as a cell-specific or tissue-specific promoter. Polypeptides also can be expressed from vectors that specifically infect and/or replicate in target cell types and/or tissues. Inducible promoters can be used to selectively regulate polypeptide or construct expression.

Nucleic acid molecules, such as naked nucleic acids, or vectors, artificial chromosomes, liposomes and other vehicles can be administered to the subject by systemic administration, topical, local and other routes of administration. When systemic and in vivo, the nucleic acid molecule or vehicle containing the nucleic acid molecule can be targeted to a cell. Administration can include intravenous administration, and direct injection into a tissue, such as direct injection into the liver, including methods of direct injection into a compartmentalized organ or portion thereof, such as the liver (see, e.g., U.S. Pat. No. 9,821,114).

Administration also can be direct, such as by administration of a vector or cells that typically targets a cell or tissue. Cells used for in vivo expression of a polypeptide or construct herein also include cells autologous to the patient. Such cells can be removed from a patient, the nucleic acids for expression of a polypeptide introduced, and then administered to a patient, such as by injection or engraftment.

### I. Compositions, Formulations and Dosages

Provided are pharmaceutical compositions containing, in a pharmaceutically acceptable vehicle, any of the polypeptides and constructs provided herein, including the TNFR1 antagonist constructs, the TNFR2 agonist constructs, and multi-specific, such as bi-specific, TNFR1 antagonist/TNFR2 agonist constructs, including fusion proteins, or nucleic acid molecules encoding the polypeptides or constructs. Such compositions contain an amount of the polypeptides, constructs, or nucleic acids that can be diluted to a therapeutically effective amount, or that are formulated for direct administration without dilution. The particular concentration of a construct or nucleic acid depends upon a variety of parameters within the skill of a skilled artisan, including, for example, the treated indication, the construct or nucleic acid, the route of administration, and the regimen. Routes of administration include systemic and local routes, oral, rectal, intravenous, intramuscular, subcutaneous, mucosal, peritoneal, and any suitable route known to the skilled person.

A selected amount, such as a therapeutically effective amount, depending, as discussed above, on various parameters of the constructs provided herein, including the TNFR1 antagonist constructs, the TNFR2 agonist constructs, and multi-specific, such as bi-specific, TNFR1 antagonist/TNFR2 agonist constructs, including fusion proteins, or nucleic acid molecules encoding the constructs, is formulated in a suitable vehicle for administration. The pharmaceutical compositions can be formulated in any conventional manner, by mixing a selected amount of a construct or mixture thereof with one or more physiologically acceptable carriers or excipients or vehicles The pharmaceutical composition can be used for therapeutic, prophylactic, and/or diagnostic applications. The concentration of the active compound, i.e., the construct or nucleic acid, in a composition, depends on a variety of factors, including those noted above, as well as the absorption, inactivation, and excretion rates of the active compound, the dosage schedule, and the amount administered, the age and size of the subject, as well as other factors known to those of skill in the art.

Pharmaceutical carriers or vehicles suitable for administration of the compounds provided herein include any such carriers known to those skilled in the art to be suitable for the particular mode of administration. Pharmaceutical compositions that include a therapeutically effective amount of a construct or nucleic acid molecule described herein also can be provided as a lyophilized powder that is reconstituted, such as with sterile water, immediately prior to administration.

### 1. Formulations

Pharmaceutical compositions containing any of the constructs and nucleic acids provided herein can be formulated in any conventional manner, by mixing a selected amount of the active compound with one or more physiologically acceptable carriers or excipients. Selection of the carrier or excipient is within the skill of the administering professional, and can depend upon a number of parameters. These include, for example, the mode of administration (i.e., systemic, oral, nasal, pulmonary, local, topical, or any other mode), and the disorder treated. Generally, the pharmaceutical compositions include components that do not significantly impair the biological properties of the constructs or nucleic acid or encoded polypeptide or that enhance or improve pharmacological properties thereof. The formulations also can be co-formulations with other active agents for combination therapy.

The pharmaceutical compositions provided herein can be in various forms, such as, but are limited to, in solid, semi-solid, liquid, emulsions, powder, aqueous, and lyophilized forms. The pharmaceutical compositions provided herein can be formulated for single dosage (direct) administration, or for dilution, or other regimen. The concentrations of the compounds in the formulations are effective, either following dilution or mixing with another composition, or for direct administration, for delivery of an amount, upon administration, that is effective for the intended treatment. The compositions can be formulated in an amount single or multiple dosage direct administration. The compound can be suspended in micronized or other suitable form, or can be derivatized to produce a more soluble active product. The form of the resulting mixture depends upon a number of factors, including the intended mode of administration, and the solubility of the compound in the selected carrier or vehicle. The resulting mixtures are solutions, suspensions, emulsions and other such mixtures, and can be formulated as non-aqueous or aqueous mixtures, creams, gels, ointments, emulsions, solutions, elixirs, lotions, suspensions, tinctures, pastes, foams, aerosols, irrigations, sprays, suppositories, bandages, or any other formulation suitable for systemic, topical or local administration. For local internal administration, such as intramuscular, parenteral or intra-articular administration, the constructs and nucleic acids can be formulated as a solution suspension in an aqueous-based medium, such as isotonically buffered saline, or are combined with a biocompatible support or bioadhesive intended for internal administration. The effective concentration is sufficient for ameliorating the targeted condition and can be empirically determined. To formulate a composition, the weight fraction of compound is dissolved, suspended, dispersed, or otherwise mixed in a selected vehicle, at an effective concentration, such that the targeted condition is relieved or ameliorated.

Generally, pharmaceutically acceptable compositions are prepared in view of approvals for a regulatory agency, or other agency, and/or are prepared in accordance with generally recognized pharmacopeia for use in animals and in humans. Pharmaceutical compositions can include a carrier, such as a diluent, adjuvant, excipient, or vehicle, with which a polypeptide is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain, along with an active ingredient, a diluent, such as lactose, sucrose, dicalcium phosphate, and carboxymethylcellulose; a lubricant, such as magnesium stearate, calcium stearate and talc; and a binder, such as starch, natural gums, such as gum acacia, gelatin, glucose, molasses, polyvinylpyrrolidone, celluloses and derivatives thereof, povidone, crospovidone, and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents. These compositions can take the form of solutions, suspensions, emulsions, tablets, pills, capsules, powders, granules, and sustained release formulations. Capsules and cartridges of, e.g., gelatin, for use in an inhaler or insufflator, can be formulated containing a powder mix of a therapeutic compound and a suitable powder base, such as lactose or starch. A composition can be formulated as a suppository, with traditional binders and carriers, such as triglycerides. Oral formulations can include standard carriers, such as pharmaceutical grades of mannitol, lactose, starch, magnesium stearate, sodium saccharine, cellulose, magnesium carbonate, and other such agents. Preparations for oral administration also can be suitably formulated with protease inhibitors, such as a Bowman-Birk inhibitor, a conjugated Bowman-Birk inhibitor, aprotinin and camostat. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier, so as to provide the compound in a form for proper administration to a subject or patient.

The pharmaceutical compositions provided herein can contain other additives, including, for example, antioxidants, preservatives, antimicrobial agents, analgesic agents, binders, disintegrants, colorings, diluents, excipients, extenders, glidants, solubilizers, stabilizers, tonicity agents, vehicles, viscosity agents, flavoring agents, emulsions, such as oil-in-water or water-in-oil emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol-9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients, such as crystalline cellulose, microcrystalline cellulose, citric acid, dextrin, dextrose, liquid glucose, lactic acid, lactose, magnesium chloride, potassium metaphosphate, and starch, among others (see, generally, Alfonso R. Gennaro (2000) Remington: The Science and Practice of Pharmacy, 20th Edition. Baltimore, MD: Lippincott Williams & Wilkins). Such carriers and/or additives can be formulated by conventional methods and can be administered to the subject at a suitable dose. Stabilizing agents, such as lipids, nuclease inhibitors, polymers, and chelating agents, can preserve the compositions from degradation within the body.

The formulation should suit the mode of administration. For example, the active compound can be formulated for parenteral administration by injection (e.g., by bolus injection, or continuous infusion). The injectable compositions can take such forms as suspensions, solutions or emulsions in oily or aqueous vehicles. The sterile injectable preparation also can be a sterile injectable solution, or a suspension in a non-toxic parenterally-acceptable diluent or solvent, for example, as a solution in 1,4-butanediol. Sterile, fixed oils are conventionally employed as a solvent or suspending medium. For this purpose, any bland fixed oil can be employed, including, but not limited to, synthetic mono- or diglycerides, fatty acids (including oleic acid), naturally occurring vegetable oils, such as sesame oil, coconut oil, peanut oil, cottonseed oil, and other oils, or synthetic fatty vehicles like ethyl oleate. Buffers, preservatives, antioxidants, and the suitable ingredients, can be incorporated as required, or, alternatively, can comprise the formulation.

The active compound, such as the constructs and nucleic acids provided herein, can be formulated as the sole pharmaceutically active ingredient in the composition, or can be combined with other active ingredients. The active compound can be targeted for delivery, such as by conjugation to a targeting agent, such as an antibody. Liposomal suspensions, including tissue-targeted liposomes, also can be suitable as pharmaceutically acceptable carriers. These can be prepared according to methods known to those skilled in the art. For example, liposome formulations can be prepared by methods well known to those of skill in the art, such as those as described in U.S. Pat. No. 4,522,811. Liposomal delivery also can include slow release formulations, including pharmaceutical matrices, such as collagen gels and liposomes modified with fibronectin (see, e.g., Weiner et al. (1985) J. Pharm. Sci. 74(9):922-925). The compositions provided herein further can contain one or more adjuvants that facilitate delivery, such as, but not limited to, inert carriers, or colloidal dispersion systems. Representative and non-limiting examples of such inert carriers can be selected from water, isopropyl alcohol, gaseous fluorocarbons, ethyl alcohol, polyvinylpyrrolidone, propylene glycol, a gel-producing material, stearyl alcohol, stearic acid, spermaceti, sorbitan monooleate, methylcellulose, as well as suitable combinations of two or more thereof.

The active compound is included in the pharmaceutically acceptable carrier in an amount sufficient to exert a therapeutically useful effect in the absence of undesirable side effects on the subject treated. The therapeutically effective concentration can be determined empirically by testing the compounds in known in vitro and in vivo systems, such as the assays described herein. Determination of a therapeutically effective amount is well within the capability of those skilled in the art. Therapeutically effective dosages can be determined by using in vitro and in vivo methods as described herein. Accordingly, an active compound, or mixtures thereof, provided herein, when in a pharmaceutical preparation, can be present in unit dose forms for administration.

2. Administration of the TNFR1 Antagonist Constructs, TNFR2 Agonist Constructs, the Multi-specific, such as Bi-Specific, Constructs and Nucleic acids

The active compounds, including the constructs provided herein, including the TNFR1 antagonist constructs, the TNFR2 agonist constructs, and multi-specific, such as bi-specific, TNFR1 antagonist/TNFR2 agonist constructs, including fusion proteins, and nucleic acid molecules encoding the constructs, can be administered by any suitable route. These include in vitro, ex vivo, or in vivo, by contacting a mixture, such as a body fluid or other tissue sample, with the active compound provided herein. For example, when administering a compound ex vivo, a body fluid, such as the vitreous, or tissue sample from a subject, can be contacted with the polypeptides that are coated on a tube or filter, such as, for example, a tube or filter in a bypass machine. When administering in vivo, the active compounds can be administered by any appropriate route, for example, orally, nasally, pulmonary, parenterally, intravenously, intradermally, intravitreally, intraretinally, subretinally, periocularly, subcutaneously, intraarticularly, intracisternally, intraocularly, intraventricularly, intrathecally, intramuscularly, intraperitoneally, intratracheally, rectally, or topically, or by direct injection into an organ, as well as by any combination of any two or more thereof, in liquid, semi-liquid or solid form, and are formulated in a manner suitable for each route of administration.

The route of administration is in accord with known methods, e.g., injection or infusion by intravenous, intraperitoneal, intracerebral, intramuscular, subcutaneous, intraocular, intraarterial, intrathecal, inhalation or intralesional routes, topical, or by sustained release systems. The antibody or fragment thereof is typically administered continuously by infusion or by bolus injection. The active compounds provided herein can be prepared in a mixture with a pharmaceutically acceptable carrier, as discussed above. Techniques for formulation and administration of the compounds are known to one of skill in the art. This therapeutic composition can be administered intravenously or through the nose or lung, such as, as a liquid or powder aerosol (lyophilized). The composition also can be administered parenterally or subcutaneously as desired. When administered systematically, the therapeutic composition should be sterile, pyrogen-free, and in a parenterally acceptable solution having due regard for pH, isotonicity, and stability, and other conditions known to those skilled in the art.

Therapeutic formulations can be administered in many conventional dosage formulations. Dosage formulations of the active compounds provided herein are prepared for storage or administration by mixing the compound having the desired degree of purity with physiologically acceptable carriers, excipients, or stabilizers. Such materials are non-toxic to the recipients at the dosages and concentrations employed, and can include buffers such as TRIS HCl, phosphate, citrate, acetate and other organic acid salts; antioxidants such as ascorbic acid; low molecular weight (less than about ten residues) peptides such as polyarginine; proteins, such as serum albumin, gelatin, or immunoglobulins; hydrophilic polymers, such as polyvinylpyrrolidone; amino acids, such as glycine, glutamic acid, aspartic acid, or arginine; monosaccharides, disaccharides, and other carbohydrates, including cellulose or its derivatives, glucose, mannose, or dextrins; chelating agents, such as EDTA; sugar alcohols, such as mannitol or sorbitol; counter ions, such as sodium and/or nonionic surfactants, such as those sold under the trademarks TWEEN and PLURONICS, and polyethylene glycol (PEG).

Pharmaceutical compositions can contain a stabilizing agent. The stabilizing agent can be an amino acid, amino acid derivative, amine, sugar, polyols, salts or surfactants. In some examples, the stable co-formulations contain a single stabilizing agent. In other examples, the stable co-formulations contain 2, 3, 4, 5 or 6 different stabilizing agents. For example, the stabilizing agent can be a sugar or polyol, such as a glycerol, sorbitol, mannitol, inositol, sucrose or trehalose. In particular examples, the stabilizing agent is sucrose. In other examples, the stabilizing agent is trehalose. The concentration of the sugar or polyol is from or from about 100 mM to 500 mM, 100 mM to 400 mM, 100 mM to 300 mM, 100 mM to 200 mM, 200 mM to 500 mM, 200 mM to 400 mM, 200 mM to 300 mM, 250 mM to 500 mM, 250 mM to 400 mM, 250 mM to 300 mM, 300 mM to 500 mM, 300 mM to 400 mM, or 400 mM to 500 mM, each inclusive.

In examples, the stabilizing agent can be a surfactant that is a polypropylene glycol, polyethylene glycol, glycerin, sorbitol, poloxamer and polysorbate. For example, the surfactant can be a polypropylene glycol, polyethylene glycol, glycerin, sorbitol, poloxamer or polysorbate, such as a poloxamer 188, polysorbate 20 and polysorbate 80. In particular examples, the stabilizing agent is polysorbate 80. The concentration of surfactant, as a % of mass concentration (w/v) in the formulation, is between or about between 0.005% to 1.0%, 0.01% to 0.5%, 0.01% to 0.1%, 0.01% to 0.05%, or 0.01% to 0.02%, each inclusive.

For in vivo administration, the formulation should be sterile. This readily is accomplished, such as by filtration through sterile filtration membranes, prior to, or following lyophilization and reconstitution. The formulation can be stored in lyophilized form or in solution. Other vehicles, such as naturally occurring vegetable oil, such as sesame, peanut, or cottonseed oil, or a synthetic fatty vehicle, such as ethyl oleate, or the like, can be used. Buffers, preservatives, antioxidants and the like can be incorporated according to accepted pharmaceutical practice.

Determination of dosage is within the skill of the physician, and can be a function of the particular disorder, route of administration and subject. Exemplary dosages, include for example 0.1 to 100 mg/kg, such as 1 to 10 mg/kg, or an appropriate amount based on the mass of the treated subject; average human subjects have a mass of about 70-75 kg. The polypeptides can be administered once or more than once, such as twice, three times, four times, or any number of times that are required to achieve a therapeutic effect. Multiple administrations can be effected via any route or combination of routes, and can be administered hourly, every 2 hours, every three hours, every four hours or more.

The active compounds can be provided at a concentration in the composition of, for example, from or from about 0.1 to 10 mg/mL, such as, for example, a concentration that is at least or at least about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 mg/mL, or more. The volume of the solution can be at or about 0.1 to 100 mL or more, such as, for example, at least or about at least or 0.5, 1, 2, 3, 4, 5, 10, 15, 20, 25, 30, 35, 40, 45, 50, 55, 60, 65, 70, 75, 80, 85, 90, 95, or 100 mL, or more. In some examples, the active compound is supplied in phosphate buffered saline. For example, the compositions can be supplied as a 50 mL, vial or other container containing 100 mg of polypeptide or fusion protein at a concentration of 2 mg/mL in phosphate buffered saline.

Active compounds provided herein, can be lyophilized for storage and reconstituted in a suitable carrier prior to use. This technique has been shown to be effective with conventional immunoglobulins and protein preparations, and art-known lyophilization and reconstitution techniques can be employed.

The active compounds provided herein, can be provided as a controlled release or sustained release composition. Polymeric materials are known in the art for the formulation of pills and capsules which can achieve controlled or sustained release (see e.g., Controlled Drug Bioavailability, Drug Product Design and Performance, Smolen and Ball (eds.), Wiley, New York (1984); see, also, Levy et al. (1985) Science 228:190; During et al. (1989) Ann. Neurol. 25:351; Howard et al. (1989) J. Neurosurg. 71:105; U.S. Pat. Nos. 5,679,377, 5,916,597, 5,912,015, 5,989,463, and 5,128,326; and International Application Publication Nos. WO 99/015154 and WO 99/020253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. Generally, the polymer used in a sustained release formulation is inert, free of leachable impurities, stable on storage, sterile, and biodegradable. Any technique known in the art for the production of sustained release formulations can be used to produce a sustained release formulation.

The constructs and nucleic acids, and physiologically acceptable forms thereof salts and solvates, can be formulated for administration by inhalation (either through the mouth or the nose), or other routes of administration, including, for example, oral, transdermal, pulmonary, parenteral or rectal administration. For administration by inhalation, the active compounds can be delivered in the form of an aerosol spray presentation from pressurized packs or a nebulizer with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin, for use in an inhaler or insufflator, can be formulated containing a powder mix of a therapeutic compound and a suitable powder base, such as lactose or starch.

For pulmonary administration to the lungs, the constructs can be delivered in the form of an aerosol spray presentation from a nebulizer, turbo nebulizer, or microprocessor-controlled metered dose oral inhaler, with the use of a suitable propellant. Generally, particle size of the aerosol is small, such as in the range of 0.5 to 5 microns. In the case of a pharmaceutical composition formulated for pulmonary administration, detergent surfactants are not typically used. Pulmonary drug delivery is a promising non-invasive method of systemic administration. The lungs represent an attractive route for drug delivery, mainly due to the high surface area for absorption, thin alveolar epithelium, extensive vascularization, lack of hepatic first-pass metabolism, and relatively low metabolic activity.

For oral administration, the pharmaceutical compositions can take the form of, for example, tablets, pills, liquid suspensions, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives, such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates, or sorbic acid). The preparations also can contain buffer salts, flavoring, coloring, and sweetening agents, as appropriate.

Preparations for oral administration can be formulated for controlled release of the active compound. For buccal administration, the compositions can take the form of tablets or lozenges formulated in a conventional manner. The active compounds can be formulated as a depot preparation. Such long-acting formulations can be administered by implantation (for example, subcutaneously or intramuscularly), or by intramuscular injection. Thus, for example, the therapeutic compounds can be formulated with suitable polymeric or hydrophobic materials (for example, as an emulsion in an acceptable oil) or ion exchange resins, or as sparingly soluble derivatives, for example, as a sparingly soluble salt.

The active compounds can be formulated for parenteral administration by injection (e.g., by bolus injection or continuous infusion). Formulations for injection can be presented in unit dosage form (e.g., in ampoules or in multi-dose containers) with an added preservative. The compositions can take such forms as suspensions, solutions or emulsions in oily or aqueous vehicles, and can contain formulatory agents, such as suspending, stabilizing and/or dispersing agents. Alternatively, the active ingredient can be in powder-lyophilized form for constitution with a suitable vehicle, e.g., sterile pyrogen-free water, before use.

The pharmaceutical compositions can be formulated for local or topical application, such as for topical application to the skin (transdermal) and mucous membranes, such as in the eye, in the form of gels, creams, and lotions, and for application to the eye or for intracisternal or intraspinal application. Such solutions, particularly those intended for ophthalmic use, can be formulated as 0.01%-10% isotonic solutions and a pH of about 5-7 with appropriate salts. The compounds can be formulated as aerosols for topical application, such as by inhalation (see, for example, U.S. Pat. Nos. 4,044,126, 4,414,209 and 4,364,923, which describe aerosols for delivery of a steroid useful for treatment of inflammatory diseases, particularly asthma).

The concentration of active compound in the drug composition depends on absorption, inactivation and excretion rates of the active compound, the dosage schedule, and amount administered as well as other factors known to those of skill in the art. As described further herein, dosages can be determined empirically using comparisons of properties and activities. For example, inhibition of TNF-mediated inflammatory signaling via TNFR1 by the constructs provided herein can be compared to traditional anti-TNF therapies, such as adalimumab.

The compositions, if desired, can be presented in a package, in a kit or dispenser device, that can contain one or more unit dosage forms containing the active ingredient. In some examples, the composition can be coated on a device, such as for example on a tube or filter in, for example, a bypass machine. The package, for example, contains metal or plastic foil, such as a blister pack. The pack or dispenser device can be accompanied by instructions for administration. The compositions containing the active agents can be packaged as articles of manufacture containing packaging material, an agent provided herein, and a label that indicates the disorder for which the agent is provided. The pharmaceutical compositions can be packaged in unit dosage forms containing an amount of the pharmaceutical composition for a single dose or multiple doses. The packaged compositions can contain a lyophilized powder of the pharmaceutical compositions containing the constructs provided herein, including the TNFR1 antagonist constructs, the TNFR2 agonist constructs, and multi-specific, such as bi-specific, TNFR1 antagonist/TNFR2 agonist constructs, including fusion proteins, and nucleic acid molecules encoding the constructs provided herein, which can be reconstituted (e.g., with water or saline) prior to administration.

The articles of manufacture provided herein contain packaging materials. Packaging materials for use in packaging pharmaceutical products are well-known to those of skill in the art (see, e.g., U.S. Pat. Nos. 5,323,907, 5,052,558 and 5,033,252). Examples of pharmaceutical packaging materials include, but are not limited to, blister packs, bottles, tubes, inhalers (e.g., pressurized metered dose inhalers (MDI), dry powder inhalers (DPI), nebulizers (e.g., jet or ultrasonic nebulizers) and other single breath liquid systems), pumps, bags, vials, containers, syringes, bottles, and any packaging material suitable for a selected formulation and intended mode of administration and treatment.

The active compounds, including the constructs provided herein, including the TNFR1 antagonist constructs, the TNFR2 agonist constructs, and multi-specific, such as bi-specific, TNFR1 antagonist/TNFR2 agonist constructs, including fusion proteins, and nucleic acid molecules encoding the constructs, the pharmaceutical compositions, and combinations of the active agents and other compositions, including other therapeutic agents, can be provided as kits. Kits can optionally include one or more components, such as instructions for use, devices, additional reagents (e.g., sterilized water or saline solutions for dilution of the compositions and/or reconstitution of lyophilized protein), and components, such as tubes, containers and syringes, for practice of the methods. Exemplary kits can include the constructs and encoding nucleic acids provided herein, and optionally can include instructions for use, a device for administering the compounds to a subject, a device for detecting the compounds in a subject, a device or devices for detecting the compounds or metabolites thereof in samples obtained from a subject, and a device for administering an additional therapeutic agent to a subject. The kit, optionally, can include instructions for use. Instructions typically include a tangible expression describing the active compound(s), and, optionally, other components included in the kit, and methods for administration, including methods for determining the proper state of the subject, the proper dosage amount, dosing regimens, and administration methods. Instructions also can include guidance for monitoring the subject over the duration of the treatment time.

Kits also can include a pharmaceutical composition described herein and an item for diagnosis. For example, such kits can include an item for measuring the concentration, amount or activity of the administered active compound in a subject. Kits provided herein also can include a devices for administering the compound(s). Any of a variety of devices known in the art for administering medications to a subject can be included in the kits provided herein. Exemplary devices include, but are not limited to, a hypodermic needle, an intravenous needle, and a catheter. Typically, a device for administering is compatible with the desired method of administration of the active agent.

### 3. Administration of Nucleic Acids Encoding Polypeptides (Gene Therapy)

Among the pharmaceutical compositions are those containing nucleic acid molecules that encode the polypeptide constructs provided herein. Rather than deliver the protein, nucleic acid molecules can be administered in vivo, such as systemically or by other route, or ex vivo, such as by removal of cells, including lymphocytes, introduction of the nucleic acid molecule therein, and reintroduction into the host or a compatible recipient.

The polypeptide constructs provided herein, including the TNFR1 antagonist constructs, the TNFR2 agonist constructs, and multi-specific, such as bi-specific, TNFR1 antagonist/TNFR2 agonist constructs, including fusion proteins, can be delivered to cells and tissues by expression of nucleic acid molecules. Nucleic acids can be delivered to cells and tissues by any method known to those of skill in the art. The isolated nucleic acid can be incorporated into vectors for further manipulation. As discussed above, methods for administering the polypeptides by expression of encoding nucleic acid molecules include administration of recombinant vectors. The vector can be designed to remain episomal, such as by inclusion of an origin of replication, or can be designed to integrate into a chromosome in the cell. The polypeptides also can be used in ex vivo gene expression therapy using non-viral vectors. Suitable gene therapy vectors and methods of delivery are known to those of skill in the art, and are discussed in sections above.

### J. Therapeutic Uses and Methods of Treatment

Pharmaceutical compositions, such as those described above, are prepared and are administered to subjects with a disease, disorder, or condition amenable to treatment with a construct that inhibits and/or agonizes TNFR1 and TNFR2, respectively. Dosage depends upon the particular disorder, disease or condition that is treated, as well as the particular subject. Typical doses are similar to known TNF blockers, such as etanercept. Exemplary doses, for a subject, including humans and other animals, range from about or 0.1 mg/kg to 100 mg/kg, such as 1 mg/kg to about or 30 mg/kg, such as 5 mg/kg to 25 mg/kg. Dose can be determined based on the assumption that an average human has a mass of about 75 kg. Doses can be adjusted for children, infants, and smaller adults.

The TNFR1 antagonists, TNFR2 agonists, bi-specific TNFR1 antagonist/TNFR2 agonist constructs and fusion proteins provided herein can be used for any purpose known to the skilled artisan for use of such molecules, including for treatment of any diseases, disorders, and conditions described herein. For example, the TNFR1 antagonists, TNFR2 agonists, multi-specific TNFR1 antagonist/TNFR2 agonist constructs, and fusion proteins provided herein can be used for one or more of therapeutic, diagnostic, industrial and/or research purpose(s). Methods of treatment provided herein include methods for the therapeutic uses of the TNFR1 antagonists, TNFR2 agonists, multi-specific TNFR1 antagonist/TNFR2 agonist constructs and fusion proteins provided herein. For example, the TNFR1 antagonists described herein can be used to antagonize TNFR1, and/or to inhibit the binding of TNF to TNFR1, and/or to inhibit TNF-mediated pro-inflammatory signaling through TNFR1. The TNFR2 agonists can be used to agonize TNFR2, in order to induce protective/anti-inflammatory TNFR2 signaling, and/or induce the expansion, proliferation and activation of immunosuppressive TNFR2⁺regulatory T-cells (Tregs). In some embodiments, as described herein, the combination of the TNFR1 antagonists and TNFR2 agonists, or the use of the bi-specific TNFR1 antagonist/TNFR2 agonist constructs provided herein, provides for the selective inhibition of pro-inflammatory TNFR1 activity, while maintaining or increasing TNFR2-associated protective signaling and Treg immunosuppressive activity, which is beneficial in the treatment of chronic inflammatory and autoimmune diseases, as well as in the treatment of neurodegenerative and demyelinating diseases and disorders.

The TNFR1 antagonists, TNFR2 agonists, multi-specific, such as bi-specific, TNFR1 antagonist/TNFR2 agonist constructs and fusion proteins provided herein can have therapeutic activity alone, or in combination with other agents. TNFR1 antagonists, TNFR2 agonists, bi-specific TNFR1 antagonist/TNFR2 agonist constructs and fusion proteins, and the encoding nucleic acid molecules, provided herein, can be used for the treatment of any condition for which anti-TNF therapies (e.g., adalimumab, infliximab, etanercept, and others described herein and/or known in the art), or other disease-modifying anti-rheumatic drugs (DMARDs; e.g., methotrexate, hydroxychloroquine, sulfasalazine, leflunomide, abatacept, anakinra, rituximab, tocilizumab, tofacitinib, and others described herein and/or known in the art) are employed, including, but not limited to, chronic inflammatory and autoimmune diseases and disorders, as well as neurodegenerative and demyelinating diseases and disorders. For example, the subject to whom the therapeutic molecules provided herein are administered exhibits acute or chronic inflammation of the joints, skin, lungs, and/or gut, and/or suffers from autoimmune diseases, rheumatoid arthritis (RA), psoriasis, psoriatic arthritis, juvenile idiopathic arthritis (JIA), spondyloarthritis, ankylosing spondylitis, Crohn's disease, ulcerative colitis, inflammatory bowel disease (IBD), uveitis, fibrotic diseases, endometriosis, lupus, multiple sclerosis, congestive heart failure, cardiovascular disease, myocardial infarction (MI), atherosclerosis, metabolic diseases, cytokine release syndrome, septic shock, sepsis, acute respiratory distress syndrome (ARDS), severe acute respiratory syndrome (SARS), COVID-19, influenza, acute and chronic neurodegenerative diseases, demyelinating diseases and disorders, stroke, Alzheimer's disease, Parkinson's disease, Behget's disease, Dupuytren's disease, Tumor Necrosis Factor Receptor-Associated Periodic Syndrome (TRAPS), pancreatitis, type I diabetes, chronic obstructive pulmonary disease (COPD), chronic bronchitis, emphysema, graft rejection, graft versus host disease (GvHD), respiratory diseases, lung inflammation, pulmonary diseases and conditions, asthma, cystic fibrosis, idiopathic pulmonary fibrosis, acute fulminant viral or bacterial infections, pneumonia, genetically inherited diseases with TNF/TNFR1 as the causative pathologic mediator, periodic fever syndrome, and cancer.

The constructs provided herein, when administered, generally can result in subjects exhibiting reduced or lessened side effects, compared to side effects that can be observed after administration of anti-TNF therapies. Treatment of diseases and conditions with the polypeptides provided herein, such as the TNFR1 antagonists, TNFR2 agonists, and the bi-specific constructs and fusion proteins thereof, can be effected by any suitable route of administration, using suitable formulations as described herein, including, but not limited to, infusion, subcutaneous injection, and inhalation, or intramuscular, intradermal, oral, topical and transdermal administration.

As discussed elsewhere herein, existing anti-TNF therapies, such as adalimumab, are immunosuppressive, due to the blockade of TNF signaling via TNFR1 and TNFR2, and are associated with a risk of adverse side effects, including, for example, an increased risk of sepsis and serious infections, such as listeriosis, reactivation of tuberculosis, reactivation of hepatitis B/C, reactivation of herpes zoster, and invasive fungal and other opportunistic infections. Anti-TNF agents also can cause worsening of severe congestive heart failure, and can cause drug-induced lupus, liver injury, psoriasis, sarcoidosis, and demyelinating central nervous system (CNS) diseases, and an increased susceptibility to the development of additional autoimmune diseases, as well as lymphomas and solid malignancies, such as non-melanoma skin cancers. Depending on the anti-TNF agent, about 3-33% of treated patients do not respond to treatment, and up to 46% stop responding, resulting in discontinuation or dose increase. Anti-TNF therapies have failed in the treatment of neurodegenerative diseases and CNS conditions, such as Alzheimer's disease, Parkinson's disease, stroke and multiple sclerosis (MS), which have been associated with the overexpression of TNF. Due to the adverse effects associated with the use of anti-TNF agents, the non-responsiveness of some patients, the lack of a sustained response in patients that had an initial response, and the failure to treat and/or the exacerbation of neurodegenerative diseases, such as MS, other therapies are needed. Such therapies are provided herein.

As described herein, the selective inhibition of TNFR1 retains the potent anti-inflammatory and protective activity of TNFR2 signaling, results in fewer opportunistic infections and cancer, and preserves TNF-induced Treg functions. Prior selective TNFR1 antagonists suffer from immunogenicity, including the formation of anti-drug antibodies (ADAs), poor pharmacokinetics and pharmacodynamics, including, for example, short serum half-life, rapid renal clearance, and/or poor binding affinity and potency. The therapies provided herein overcome the limitations associated with prior selective TNFR1 antagonists. Examples of therapeutic improvements using the polypeptides provided herein, such as the TNFR1 antagonists, include, but are not limited to, lower dosages, fewer and/or less frequent administrations, decreased side effects and increased therapeutic effects.

Hence, the selective TNFR1 antagonists, TNFR2 agonists, and multi-specific, such as bi-specific TNFR1 antagonist/TNFR2 agonist constructs, and fusion proteins, provided herein, are associated with reduced side effects, can be used, if necessary, at higher dosing regimens, and can have improved efficacy and safety. Side effects that can be reduced, lessened or eliminated, compared to those observed by existing anti-TNF therapeutics, such as adalimumab and others described herein and/or known in the art, include any undesirable non-therapeutic effect described herein or known in the art, such as, but not limited to, sepsis, serious infections, congestive heart failure/cardiotoxicity, generation of antibodies, and the development or worsening of cancer, autoimmune disease and/or demyelinating central nervous system (CNS) disease. In some examples, compared to side effects caused by administration of existing anti-TNF therapeutics, such as adalimumab, administration of a TNFR1 antagonist, bi-specific construct, or fusion protein provided herein decreases the severity of one or more side effects by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10%, relative to the severity of the one or more side effects of an anti-TNF therapy.

Dosage levels and regimens can be determined based upon known dosages and regimens, and, if necessary can be extrapolated based upon the changes in properties of the polypeptides and constructs provided herein, and/or can be determined empirically based on a variety of factors. Such factors include, for example, the body weight of the individual, as well as their general health, age, sex, and diet, and the activity of the specific compound employed, the time of administration, the rate of excretion, drug combinations, the severity and course of the disease, and the patient's disposition to the disease and the judgment of the treating physician. The active ingredient typically is combined with a pharmaceutically effective carrier. The amount of active ingredient that can be combined with the carrier materials to produce a single dosage form or multi-dosage form can vary depending upon the host treated and the particular mode of administration.

Upon improvement of a patient's condition, a maintenance dose of a compound or composition can be administered, if necessary; and the dosage, the dosage form, or frequency of administration, or a combination thereof, can be modified. In some cases, a subject can require intermittent treatment on a long-term basis upon any recurrence of disease symptoms, or based upon scheduled dosages.

This section provides exemplary uses and administration methods for the constructs, include polypeptides and encoding nucleic acid molecules provided herein. These described therapies are exemplary only and do not limit the applications of the molecules/constructs provided herein. It is within the skill of a treating physician to identify diseases or conditions which are treatable using a TNFR1 antagonist, TNFR2 agonist, bi-specific TNFR1 antagonist/TNFR2 agonist construct, and fusion protein, as well as an encoding nucleic acid molecule, provided herein.

### 1. Treatment of Chronic Inflammatory/Autoimmune Diseases and Disorders

As described herein, elevated levels or uncontrolled expression of TNF, as well as deregulation of TNF signaling, can cause chronic inflammation, which can result in the development of autoimmune diseases and tissue damage. TNF signaling via TNFR1 is primarily pro-inflammatory, and drives the development of chronic inflammatory and autoimmune diseases and disorders. For example, TNFR1 signaling is associated with the development of arthritis, inflammatory bowel disease (IBD), and respiratory diseases, among others, as well as with the generation of osteoclasts which results in local bone destruction, and cardiotoxic effects in TNF-induced models of heart failure and myocardial infarction. Thus, the selective blockade of TNFR1 is useful in the treatment of chronic inflammatory and autoimmune diseases and conditions. TNF signaling via TNFR2, which primarily is anti-inflammatory, has been associated with neuro-, cardio-, gut- and osteo-protective effects. The anti-inflammatory and protective effects of TNFR2 signaling have been demonstrated in, for example, experimental colitis, heart failure/heart disease, myocardial infarction, inflammatory arthritis, infectious disease, pancreatic regeneration, stem cell proliferation, the destruction of autoreactive T-cells, and the regulation of osteoclastogenesis for maintaining bone mass, and protecting against joint inflammation and erosive destruction. TNFR2 agonism also results in the proliferation and expansion of immunosuppressive TNFR2⁺Tregs, and promotes Treg cell suppressive activity, which eliminates autoreactive/effector T-cells, prevents tissue destruction, and suppresses inflammatory and autoimmune diseases and conditions. TNFR2 agonism, thus, also can be used to treat or alleviate the symptoms of chronic inflammatory and autoimmune diseases and conditions.

The TNFR1 antagonists, TNFR2 agonists, multi-specific TNFR1 antagonist/TNFR2 agonist constructs, fusion proteins, and encoding nucleic acids provided herein can be used to treat or alleviate the symptoms of autoimmune/inflammatory diseases and disorders associated with elevated TNF levels and deregulated TNF signaling. The constructs, fusion proteins and nucleic acids, provided herein, can be used for treating diseases, disorders, and conditions, including, but not limited to, for example, arthritis (e.g., rheumatoid arthritis, psoriatic arthritis, juvenile idiopathic arthritis, spondyloarthritis), inflammatory bowel disease (e.g., Crohn's disease and ulcerative colitis), uveitis, fibrotic diseases (e.g., Dupuytren's disease), Behget's disease, endometriosis, lupus, ankylosing spondylitis, psoriasis, tumor necrosis factor receptor-associated periodic syndrome (TRAPS), cardiovascular disease, congestive heart failure, myocardial infarction (MI), atherosclerosis, respiratory diseases, asthma, cystic fibrosis, chronic obstructive pulmonary disease (COPD), pancreatitis, type I diabetes, metabolic diseases, cytokine release syndrome, septic shock, sepsis, acute respiratory distress syndrome (ARDS), severe acute respiratory syndrome (SARS), COVID-19, influenza, chronic bronchitis, emphysema, lung inflammation, idiopathic pulmonary fibrosis, graft rejection, graft versus host disease (GvHD), acute fulminant viral or bacterial infections, pneumonia, genetically inherited diseases with TNF/TNFR1 as the causative pathologic mediator, and periodic fever syndrome, among others.

The TNF blockade also can decrease a cytokine storm observed in some viral infections, such as from the SARS viruses and the infection COVID-19. This can prevent ventilator dependence, multiorgan damage, and death in patients with severe acute respiratory syndrome (SARS), such as that resulting from SARS-CoV-2 and other SARS viruses/coronaviruses. TNF-induced viral syndrome (TIVS) is induced by a TNF-driven cytokine storm that involves not only damage to the lungs, but also multiple organ failure. TIVS is analogous to SIRS (serious inflammatory respiratory syndrome), SARS (severe acute respiratory syndrome), and septicemia (caused by bacteria). These conditions impact lung function, but also many other organs. Severe acute respiratory syndrome coronavirus-2 (SARS-CoV-2, which causes COVID-19) gains access to host cells via angiotensin-converting enzyme, which is expressed in the type II surfactant-secreting alveolar cells of the lungs. Severe COVID-19 is associated with a major immune inflammatory response with abundant neutrophils, lymphocytes, macrophages, and immune mediators. Deaths from COVID-19 primarily result from diffuse alveolar damage with pulmonary edema, hyaline membrane formation, and interstitial mononuclear inflammatory infiltrate compatible with early-phase adult respiratory distress syndrome (ARDS). TNF is present in blood and disease tissues of patients with COVID-19; TNF is involved in almost all acute inflammatory reactions, acting as an amplifier of inflammation. Thus, treatments with the constructs herein are provided.

### 2. Treatment of Neurodegenerative and Demyelinating Diseases and Disorders

As discussed elsewhere herein, several neurodegenerative and demyelinating diseases and conditions are associated with chronically elevated levels of TNF in the central nervous system (CNS). Elevated levels of TNF, and TNF signaling via TNFR1, are implicated in initiating and maintaining neuroinflammation, and in promoting neuronal cell death, demyelination and cognitive decline. For example, in patients with Alzheimer's disease (AD), TNF promotes microglial activation, synaptic dysfunction, neuronal cell death, and accumulation of plaques and tangles, and, elevated levels of TNF inhibit phagocytosis of amyloid beta (A3) in the brains of AD patients, which hinders efficient plaque removal by microglia. In patients with Parkinson's disease (PD), elevated TNF levels result in neuroinflammation and dopaminergic neuron toxicity. Elevated levels of TNF, as well as polymorphisms in the gene encoding TNFR1, are linked to demyelination, and the development of demyelinating disorders, such as multiple sclerosis (MS). The selective blockade of TNF signaling via TNFR1, thus, can be used in the treatment or alleviation of neurodegenerative and demyelinating diseases and disorders, and other conditions of the CNS.

TNF signaling via TNFR2 is associated with anti-inflammatory and neuroprotective effects. For example, activation of TNFR2 by TNF inhibits seizures, attenuates cognitive dysfunction following brain injury, and promotes remyelination, as well as the survival of neurons. The proliferation, expansion and activation of immunosuppressive Tregs, following TNFR2 agonism, also has neuroprotective effects. For example, TNFR2 signaling promotes Treg cell expansion and suppressive activity in experimental autoimmune encephalomyelitis (EAE), an animal model of inflammatory CNS demyelinating disease, such as multiple sclerosis. TNFR2 agonism, thus, also can be used in the treatment or alleviation of neurodegenerative and demyelinating diseases and disorders, and other conditions of the CNS.

The TNFR1 antagonist constructs, TNFR2 agonist constructs, multi-specific, such as bi-specific, TNFR1 antagonist/TNFR2 agonist constructs, fusion proteins, and nucleic acids provided herein can be used to treat or ameliorate the symptoms of neurodegenerative and demyelinating diseases, and other CNS disorders and conditions, including, but not limited to, Alzheimer's disease, Parkinson's disease, multiple sclerosis, and stroke.

3. Treatment of Cancer and other Immunosuppressing Diseases, Disorders, and Conditions

As described herein, tumors are infiltrated by large numbers of immunosuppressive TNFR2⁺ Tregs, which prevent the proliferation of tumor-killing CD8⁺ cytotoxic T lymphocytes (CTLs) (also known as effector T-cells (Teffs)), allowing for tumor growth. Antagonism of TNFR2 on lymphocytes in the tumor microenvironment (TME) restores the balance between the two types of T-cells, by eliminating Tregs, and allowing for the activation and expansion of effector T-cells, resulting in tumor cell lysis (see, e.g., Vanamee et al. (2017) Trends in Molecular Medicine 23(11):P1037-P1046).

TNFR2 is abundantly expressed on the surfaces of many types of human cancer cells, including, for example, renal cell carcinoma, colon cancer, Hodgkin's lymphoma, multiple myeloma, cutaneous non-Hodgkin's lymphoma, and ovarian cancer. TNFR2 mutations in cancer are associated with gene duplications and constitutive activation. Murine myeloid-derived suppressor cells (MDSCs) also express TNFR2, and its inhibition has been shown to control metastasis in a murine liver cancer model. Additionally, immune checkpoint inhibitors result in the upregulation of TNFR2 on tumor-infiltrating Tregs, leading to tumor immune escape and drug resistance. Not all patients respond to therapy with immune checkpoint inhibitors, patients can relapse, and serious autoimmune side effects have been observed with checkpoint inhibitor therapy (see, e.g., Vanamee et al. (2017) Trends in Molecular Medicine 23(11):P1037-P1046). Thus, blockade of TNFR2 can be used for the treatment of certain types of cancers, by directly killing tumor cells, via the inhibition of immunosuppressive Tregs, which allows for the proliferation of effector T-cells, and by the inhibition of MDSCs, which can prevent the formation of metastases. Because TNFR2 also is expressed on normal tissues (especially macrophages; see, e.g., proteinatlas.org/ensg00000028137-tnfrsflb/tissue), the TNFR2 antagonist does not have ADCC activity, but does have FcRn activity (or enhanced FcRn activity). Administration is personalized, since, as described herein, to qualify for treatment, the patient's tumor must have a significantly higher level of TNFR2 than adjacent normal tissues. For this purpose, the TNFR2 antagonist will be used together with other therapies, especially immunomodulating treatments that otherwise lead to an accumulation of regulatory T-cells in the tumor.

The TNFR2 agonists, bi-specific TNFR1 antagonist/TNFR2 agonist constructs, and fusion proteins provided herein, thus, also can be used in the treatment of solid cancers, hematological malignancies, and other hyperproliferative diseases and disorders, including, but not limited to, for example, renal cell carcinoma, colon cancer, Hodgkin's lymphoma, multiple myeloma, cutaneous non-Hodgkin's lymphoma, and ovarian cancer.

### 4. Combination Therapies

Combination therapies include administration of the TNFR1 antagonist constructs, TNFR2 agonist constructs, multi-specific, such as bi-specific, TNFR1 antagonist/TNFR2 agonist constructs, fusion proteins, and nucleic acids provided herein, in combination with another agent or treatment, including radiation and surgery. The further agent or therapy can be administered concurrently, before, after, or intermittently with the treatments provided herein. They can be in separate compositions, or in co-formulations.

The TNFR1 antagonist constructs, TNFR2 agonist constructs, multi-specific, such as bi-specific, TNFR1 antagonist/TNFR2 agonist constructs, fusion proteins, and nucleic acids provided herein can be administered before, after, intermittently with, or concomitantly with, one or more other therapeutic regimens or agents, including, but not limited to, TNF antagonists/blockers, antibodies, cytotoxic agents, anti-inflammatory agents, cytokines, growth factors, growth inhibitory agents, cardioprotectants, immunosuppressive agents, chemotherapeutic agents, biologic or non-biologic disease-modifying anti-rheumatic drugs (DMARDs), treatments (including antibodies) for infectious diseases, or other therapeutic agents. The TNFR1 antagonist constructs, TNFR2 agonist constructs, multi-specific, such as bi-specific, TNFR1 antagonist/TNFR2 agonist constructs, and nucleic acids provided herein can be administered to the patient as a first-line treatment, or as a second-line therapy where anti-TNF therapeutics were not effective, either as acute or chronic treatments. Exemplary of anti-TNF therapies that can be used in combination therapies herein include, for example, conventional synthetic DMARDs, such as, for example (generic name and exemplary trademark): methotrexate (MTX), hydroxychloroquine (HCQ; Plaquenil®), sulfasalazine (Azulfidine®), and leflunomide (Arava®); biologic DMARDs, such as, for example, abatacept (Orencia®), anakinra (Kineret®), rituximab (Rituxan®, Truxima®, MabThera®), tocilizumab (atlizumab, Actemra®, RoActemra®), corticosteroids (e.g., dexamethasone, methylprednisolone, prednisolone, prednisone, or triamcinolone), tofacitinib (Xeljanz®), and TNF-inhibitors/anti-TNF agents, such as, for example, certolizumab pegol (Cimzia®), infliximab (Remicade®), adalimumab (Humira®), golimumab (Simponi®), and etanercept (Enbrel®). The combination therapy also can include immunotherapeutic drugs, such as, for example, cyclosporine, methotrexate, adriamycin or cisplatinum, and immunotoxins.

Examples of anti-inflammatory drugs and agents useful for combination therapies include non-steroidal anti-inflammatory drugs (NSAIDs), including salicylates, such as aspirin, traditional NSAIDs, such as ibuprofen, naproxen, ketroprofen, nabumetone, piroxicam, diclofenac, or indomethacin, and Cox-2 selective inhibitors, such as celecoxib (sold under the trademark Celebrex®), or Rotecoxin (sold under the trademark Vioxx®). Other compounds useful in combination therapies include antimetabolites, such as methotrexate and leflunomide; corticosteroids or other steroids, such as cortisone, dexamethasone, or prednisone; analgesics, such as acetaminophen; aminosalicylates, such as mesalamine; and cytotoxic agents, such as azathioprine (sold under the trademark Imuran®), cyclophosphamide (sold under the trademark Cytoxan®), and cyclosporine A.

Additional agents that can be used in combination therapies include biological response modifiers, including, for example, anti-inflammatory cytokines, such as IL-10; B-cell targeting agents, such as anti-CD20 antibodies (e.g., rituximab); compounds targeting T antigens; adhesion molecule blockers; chemokine receptor antagonists; kinase inhibitors, such as inhibitors of mitogen-activated protein (MAP) Kinase, c-Jun N-terminal Kinase (JNK), or NFκB; and peroxisome proliferator-activated receptor-gamma (PPAR-7) ligands. Additional agents that can be used in combination therapies include immunosuppressants. Immunosuppressants can include, for example, tacrolimus or FK-506; mycophenolic acid; calcineurin inhibitors (CNIs); CsA; and sirolimus, or other agents known to suppress the immune system.

The polypeptides and constructs provided herein also can be used in combination with agents that are administered to treat cardiovascular disease and/or administered during procedures to treat cardiovascular disease, such as, for example, anti-coagulants. Exemplary anti-coagulants include, but are not limited to, heparin, warfarin, acenocoumarol, phenindione, EDTA, citrate, oxalate, and direct thrombin inhibitors, such as argatroban, lepirudin, bivalirudin, and ximelagatran.

The polypeptides and constructs provided herein can be administered with an antibody for the treatment of autoimmune or inflammatory disease, transplant rejection, or GvHD. Examples of such antibodies include, but are not limited to, anti-α4β7 integrin antibodies, such as LDP-02; anti-beta2 integrin antibodies, such as LDP-O1; anti-complement (C5) antibodies such as, 5G1.1; anti-CD2 antibodies, such as BTI-322 and MEDI-507; anti-CD3 antibodies, such as OKT3 and SMART anti-CD3; anti-CD4 antibodies, such as IDEC-151, MDX-CD4 and OKT4A; anti-CD11a antibodies; anti-CD14 antibodies, such as IC14; anti-CD18 antibodies; anti-CD23 antibodies, such as IDEC 152; anti-CD25 antibodies, such as daclizumab; anti-CD40L antibodies, such as 5c8, ruplizumab and IDEC-131; anti-CD64 antibodies, such as MDX-33; anti-CD80 antibodies, such as IDEC-114; anti-CD147 antibodies, such as ABX-CBL; anti-E-selectin antibodies, such as CDP850; anti-gpIIb/IIIa antibodies, such as ReoPro®/Abcixima; anti-ICAM-3 antibodies, such as ICM3; anti-ICE antibodies, such as VX-740; anti-FcγR1 antibodies, such as MDX-33; anti-IgE antibodies, such as rhuMAb-E25; anti-IL-4 antibodies, such as SB-240683; anti-IL-5 antibodies, such as SB-240563 and SCH55700; anti-IL-8 antibodies, such as ABX-IL8; anti-interferon gamma antibodies; anti-TNF-α antibodies, such as CDP571, CDP870, D2E7, Infliximab and MAK-195F; and anti-VLA-4 antibodies, such as Antegren®. Examples of other Fc-containing molecules that can be co-administered to treat autoimmune or inflammatory disease, transplant rejection and GvHD include, but are not limited to, the TNFR2-Fc fusion protein Enbrel® (etanercept), and Regeneron's IL-1 trap.

Examples of antibodies that can be co-administered to treat infectious diseases include, but are not limited to, anti-anthrax antibodies, such as ABthrax; anti-CMV antibodies, such as CytoGam and sevirumab; anti-cryptosporidium antibodies, such as CryptoGAM and Sporidin-G; anti-helicobacter antibodies, such as Pyloran; anti-hepatitis B antibodies, such as HepeX-B and Nabi-HB; anti-HIV antibodies, such as HRG-214; anti-RSV antibodies, such as felvizumab, HNK-20, palivizumab, and RespiGam; and anti-staphylococcus antibodies, such as Aurexis, Aurograb, BSYX-A110, and SE-Mab.

In some examples, the TNFR1 antagonist constructs, TNFR2 agonist constructs, multi-specific, such as bi-specific, TNFR1 antagonist/TNFR2 agonist constructs, fusion proteins, and nucleic acids provided herein are administered with one or more chemotherapeutic agents. Examples of chemotherapeutic agents include, but are not limited, to alkylating agents, such as thiotepa and cyclophosphamide (CYTOXAN®); alkyl sulfonates, such as busulfan, improsulfan and piposulfan; androgens, such as calusterone, dromostanolone propionate, epitiostanol, mepitiostane, and testolactone; anti-adrenals, such as aminoglutethimide, mitotane, and trilostane; anti-androgens, such as flutamide, nilutamide, bicalutamide, leuprolide, and goserelin; antibiotics, such as aclacinomycins, actinomycin, anthramycin, azaserine, bleomycins, cactinomycin, calicheamicin, carubicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti estrogens, including, for example, tamoxifen, raloxifene, aromatase inhibiting 4(5)-imidazoles, 4-hydroxytamoxifen, trioxifene, keoxifene, LY 117018, onapristone, and toremifene (Fareston); anti-metabolites, such as methotrexate and 5-fluorouracil (5-FU); folic acid analogs, such as denopterin, methotrexate, pteropterin, and trimetrexate; aziridines, such as benzodepa, carboquone, meturedepa, and uredepa; ethylenimines and methylmelamines, including altretamine, triethylenemelamine, triethylenephosphoramide, triethylenethiophosphoramide and trimethylol melamine; folic acid replenishers, such as folinic acid; nitrogen mustards, such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, and uracil mustard; nitrosoureas, such as carmustine, chlorozotocin, fotemustine, lomustine, nimustine, ranimustine; platinum analogs, such as cisplatin and carboplatin; vinblastine; platinum; proteins, such as arginine deiminase and asparaginase; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, floxuridine, and 5-FU; taxanes, such as paclitaxel (TAXOL®, Bristol-Myers Squibb Oncology, Princeton, N.J.) and docetaxel (TAXOTERE®, Rhone-Poulenc Rorer, Antony, France); topoisomerase inhibitors, such as RFS 2000; thymidylate synthase inhibitors, such as Tomudex; additional chemotherapeutics, including aceglatone; aldophosphamide glycoside; aminolevulinic acid; amsacrine; bestrabucil; bisantrene; edatrexate; defosfamide; demecolcine; diaziquone; difluoromethylornithine (DMFO); eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide K (PSK, Krestin); razoxane; sizofiran; spirogermanium; tenuazonic acid; triaziquone; 2,2′, 2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; chlorambucil; gemcitabine; 6-thioguanine; mercaptopurine; methotrexate; etoposide (VP-16); ifosfamide; mitomycin C; mitoxantrone; vincristine; vinorelbine; Navelbine; Novantrone; teniposide; daunomycin; aminopterin; Xeloda; ibandronate; CPT-11; retinoic acid; esperamycins; capecitabine; and topoisomerase inhibitors, such as irinotecan. Pharmaceutically acceptable salts, acids or derivatives of any of the above can also be used.

A chemotherapeutic agent can be administered as a prodrug. Examples of prodrugs that can be administered with a TNFR1 antagonist constructs, TNFR2 agonist constructs, multi-specific, such as bi-specific, TNFR1 antagonist/TNFR2 agonist constructs, fusion proteins, and nucleic acids provided herein include, but are not limited to, for example, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxy acetamide-containing prodrugs, or optionally substituted phenylacetamide-containing prodrugs, and 5-fluorocytosine and other 5-fluorouridine prodrugs which can be converted into the more active cytotoxic free drug. The TNFR1 antagonist constructs, TNFR2 agonist constructs, multi-specific, such as bi-specific, TNFR1 antagonist/TNFR2 agonist constructs can be provided as prodrugs by, for example, linking them to a targeting agent, which targets a particular tissue or locus of disease, with an in vivo a cleavable linker, whereby the active form of the construct is released.

In some examples, a TNFR1 antagonist constructs, TNFR2 agonist constructs, multi-specific, such as bi-specific, TNFR1 antagonist/TNFR2 agonist constructs, fusion proteins, and nucleic acids provided herein is administered with one or more antibiotics, including, but not limited to: aminoglycoside antibiotics (e.g., apramycin, arbekacin, bambermycins, butirosin, dibekacin, gentamicin, kanamycin, neomycin, netilmicin, paromomycin, ribostamycin, sisomicin, and spectinomycin), aminocyclitols (e.g., spectinomycin), amphenicol antibiotics (e.g., azidamfenicol, chloramphenicol, florfenicol, and thiamphenicol), ansamycin antibiotics (e.g., rifamide and rifampin), carbapenems (e.g., imipenem, meropenem, and panipenem); cephalosporins (e.g., cefaclor, cefadroxil, cefamandole, cefatrizine, cefazedone, cefozopran, cefpimizole, cefpiramide, cefpirome, cefprozil, cefuroxime, cefixime, cephalexin, and cephradine), cephamycins (e.g., cefbuperazone, cefoxitin, cefminox, cefmetazole, and cefotetan); lincosamides (e.g., clindamycin, and lincomycin); macrolide (e.g., azithromycin, brefeldin A, clarithromycin, erythromycin, roxithromycin, and tobramycin), monobactams (e.g., aztreonam, carumonam, and tigemonam); mupirocin; oxacephems (e.g., flomoxef, latamoxef, and moxalactam); penicillins (e.g., amdinocillin, amdinocillin pivoxil, amoxicillin, bacampicillin, benzylpenicillinic acid, benzylpenicillin sodium, epicillin, fenbenicillin, floxacillin, penamecillin, penethamate hydriodide, penicillin o-benethamine, penicillin 0, penicillin V, penicillin V benzoate, penicillin V hydrabamine, penimepicycline, and phenethicillin potassium); polypeptides (e.g., bacitracin, colistin, polymixin B, teicoplanin, and vancomycin); quinolones (e.g., amifloxacin, cinoxacin, ciprofloxacin, enoxacin, enrofloxacin, fleroxacin, flumequine, gatifloxacin, gemifloxacin, grepafloxacin, lomefloxacin, moxifloxacin, nalidixic acid, norfloxacin, ofloxacin, oxolinic acid, pefloxacin, pipemidic acid, rosoxacin, rufloxacin, sparfloxacin, temafloxacin, tosufloxacin, and trovafloxacin); rifampin; streptogramins (e.g., quinupristin, and dalfopristin); sulfonamides (e.g., sulfanilamide, and sulfamethoxazole); and tetracyclines (e.g., chlortetracycline, demeclocycline hydrochloride, demethylchlortetracycline, doxycycline, Duramycin, minocycline, neomycin, oxytetracycline, streptomycin, tetracycline, and vancomycin).

In some examples, the TNFR1 antagonist constructs, TNFR2 agonist constructs, multi-specific, such as bi-specific, TNFR1 antagonist/TNFR2 agonist constructs, fusion proteins, and nucleic acids provided herein can be administered with one or more anti-fungal agents, including, but not limited to, amphotericin B, ciclopirox, clotrimazole, econazole, fluconazole, flucytosine, itraconazole, ketoconazole, miconazole, nystatin, terbinafine, terconazole, and tioconazole. In some examples, a TNFR1 antagonist constructs, TNFR2 agonist constructs, multi-specific, such as bi-specific, TNFR1 antagonist/TNFR2 agonist constructs, and nucleic acids provided herein is administered with one or more antiviral agents, including, but not limited to, protease inhibitors, reverse transcriptase inhibitors, and others, including type I interferons, viral fusion inhibitors, neuraminidase inhibitors, acyclovir, adefovir, amantadine, amprenavir, clevudine, enfuvirtide, entecavir, foscarnet, ganciclovir, idoxuridine, indinavir, lopinavir, pleconaril, ribavirin, rimantadine, ritonavir, saquinavir, trifluridine, vidarabine, and zidovudine.

The TNFR1 antagonist constructs, TNFR2 agonist constructs, multi-specific, such as bi-specific, TNFR1 antagonist/TNFR2 agonist constructs, fusion proteins, and nucleic acids provided herein can be administered in combination with the constructs described below, and also with any of the therapeutic anti-TNF agents and treatments set forth below, for combination therapies with growth factor trap constructs.

Pharmaceutical compositions containing the TNFR1 antagonist constructs, TNFR2 agonist constructs, multi-specific, such as bi-specific, TNFR1 antagonist/TNFR2 agonist constructs, fusion proteins, and nucleic acids provided herein can be used to treat any diseases, disorders, and conditions described herein, or known to those of skill in the art. The diseases, disorders, and conditions include one or more chronic inflammatory, autoimmune, neurodegenerative or demyelinating diseases or conditions. Also provided are combinations of the polypeptides and constructs provided herein, and another treatment or compound, for treatment of a chronic inflammatory, autoimmune, neurodegenerative or demyelinating disease or condition. The TNFR1 antagonist constructs, TNFR2 agonist constructs, multi-specific, such as bi-specific, TNFR1 antagonist/TNFR2 agonist constructs, fusion proteins, and nucleic acids provided herein, and the additional agent(s) can be packaged as separate compositions for administration together, or sequentially, or intermittently. Alternatively, they can be provided as a single composition for administration, or as two compositions for administration as a single composition. The combinations can be packaged as kits, optionally with additional reagents, instructions for use, vials and other containers, syringes and other items for use for treatment.

### K. Examples

The following examples are included for illustrative purposes only and are not intended to limit the scope of the invention.

### Example 1

Expression and Evaluation of Candidate Univalent TNFR1 antagonist Molecules Expression and Purification of Anti-TNFR1 Molecules

Candidate univalent TNFR1 antagonist molecules are expressed in mammalian cells, under control of a CMV promoter, using the expression plasmid depicted in FIG. 1 (where TE19080L is the inserted fragment). For the expression of protein therapeutics, such as the constructs herein, mammalian cells, such as Chinese hamster ovary (CHO) or human embryonic kidney 293 (HEK293) cells, are used to provide post-translation modification, including glycosylation, which can be important for proper protein structure, function and activity. Expression in bacterial, yeast or insect cells results, either in no glycosylation (for bacterial cells), or in different glycosylation patterns compared to expression in mammalian cells (for yeast or insect cells). Expression in bacteria also can result in the contamination of the protein therapeutic with bacterial endotoxins, which can activate innate immune cells, complicate the analysis of cell-based assays, and lead to pyrogenic effects upon in vivo administration.

Expression plasmid construction, as well as transient and stable cell line expression is performed using the methods, such as those described in Vazquez-Lombardi et al. (2018) Nat. Protoc. 13(1):99-117. Five exemplary TNFR1 antagonist molecules are produced for initial evaluation. The TNFR1 antagonist molecules include an H398-derived scFv (SEQ ID NO:678), containing one V_{L} and one V_{H} domain of H398, linked together by a (GGGGS)₃ peptide linker; the TNFR1 antagonist domain antibody (dAb) DOM1 h-574-16 (SEQ ID NO:57); the TNFR1 antagonist dAb DOM1 h-549 (SEQ ID NO:58); a fusion protein (SEQ ID NO:704), containing the TNFR1 antagonist dAb DOM1 h-574-208 (SEQ ID NO:54) from DMS5541 (described elsewhere herein), fused to the anti-serum albumin dAb (albudAb) of DMS5541 (DOM7 h-11-3; SEQ ID NO:52) by a (GGGGS)₃ peptide linker; and a fusion protein (SEQ ID NO:705), containing the anti-TNFR1 dAb designated DOM1 h-131-206 (SEQ ID NO:59), fused to the anti-serum albumin dAb (albudAb or DOM7 h-11-3; SEQ ID NO:52) of DMS5541 by a (GGGGS)₃ peptide linker. The insertion of a (GGGGS)₃ linker in the H398 scFv, and in the DOM1 h-574-208 and DOM1 h-131-206 fusion proteins, provides more flexibility between the V_{H} and VL domains, and the two dAbs, respectively, and increases the stability and resistance to denaturation of the molecules, improving the manufacturing process. The sequences of each of these TNFR1 antagonist molecules is provided in Table 12, below, some with various linker sequences (see, also Enever et al., (2015) Protein Engineering, Design & Selection 28(3):59-66, which describes dAbs and modifications thereof that can be used for further modification and addition of linkers and modifiers).

TABLE 12

| | | SEQ |
|---|---|---|
| TNFR1 antagonist | | ID |
| Molecule | Sequence | NO. |
| H398-derived scFv | QVQLQESGAELARPGASVKLSCKASGYTFTDFYINWVKQRTG | 678 |
| | QGLEWIGEIYPYSGHAYYNEKFKAKATLTADKSSSTAFMQLNS | |
| | LTSEDSAVYFCVRWDFLDYWGQGTTLTVSSGGGGSGGGGSGG | |
| | GGSDIVMTQSPLSLPVSLGDQASISCRSSQSLLHSNGNTYLHWY | |
| | VQKPGQSPKLLIYTVSNRFSGVPDRFSGSGSGTDFTLKISRVEAE | |
| | DLGVYFCSQSTHVPYTFGGGTKLEIKR | |
| DOMIh-574-16 | EVQLLESGGGLVQPGGSLRLSCAASGFTFVKYSMGWVRQAPG | 57 |
| | KGPEWVSQISNTGDRTYYADSVKGRFTISRDNSKNTLYLQMNS | |
| | LRAEDTAVYYCAIYTGRWEPFDYWGQGTLVTVSS | |
| DOMIh-549 | EVQLLESGGGLVQPGGSLRLSCAASGFTFVDYEMHWVRQAPG | 58 |
| | KGLEWVSSISESGTTTYYADSVKGRFTISRDNSKNTLYLQMNSL | |
| | RAEDTAVYYCAKRRFSASTFDYWGQGTLVTVSS | |
| DOM1h-574-208 - | EVQLLESGGGLVQPGGSLRLSCAASGFTFDKYSMGWVRQAPG | 704 |
| albudAb fusion | KGLEWVSQISDTADRTYYAHAVKGRFTISRDNSKNTLYLQMNS | |
| protein | LRAEDTAVYYCAIYTGRWVPFEYWGQGTLVTVSSGGGGSGGG | |
| | GSGGGGSDIQMTQSPSSLSASVGDRVTITCRASRPIGTTLSWYQ | |
| | QKPGKAPKLLILWNSRLQSGVPSRFSGSGSGTDFTLTISSLQPED | |
| | FATYYCAQAGTHPTTFGQGTKVEIKR | |
| DOM1h-131-206 | EVQLLESGGGLVQPGGSLRLSCAASGFTFAHETMVWVRQAPG | 705 |
| dAb - albudAb | KGLEWVSHIPPDGQDPFYADSVKGRFTISRDNSKNTLYLQMNS | |
| fusion protein | LRAEDTAVYHCALLPKRGPWFDYWGQGTLVTVSSGGGGSGG | |
| | GGSGGGGSDIQMTQSPSSLSASVGDRVTITCRASRPIGTTLSWY | |
| | QQKPGKAPKLLILWNSRLQSGVPSRFSGSGSGTDFTLTISSLQPE | |
| | DFATYYCAQAGTHPTTFGQGTKVEIKR | |

Also provided are nanobodies and constructs containing nanobodies that comprise two heavy chains selected from among those set forth in any of SEQ ID NOs: 53-83 and 503-671, such as SEQ ID NOs: 57-59, and variants thereof having at least 9500, 9600, 970%, 980%, 990% sequence identity thereto. Constructs are provided that comprise identical heavy chains. Exemplary of these is the TNFR1 dAb designated DOM1h-131-206. Also provided are constructs containing any of these dAbs, such as those that are linked, directly or, more generally, via a linker, such as a GS linker to human serum albumin or provided as an Fc fusion, or in any of the other constructs described herein.

These and other such dAbs and TNFR1 binding molecules can be modified to increase specificity for TNFR1 by eliminating any antagonistic activity for TNFR2, and/or to increase or add TNFR2 agonist activity, and/or can be modified to reduce or eliminate immunogenic epitopes, and/or can be linked to activity modifiers, such Fc units and modified Fc units/modified Fc dimers, and/or serum half-life extending moieties. The dAbs and other TNFR1 binding molecules are those that do not have TNFR1 agonist activity. They can be identified by the THP1 assay exemplified herein, or they can be identified as those that bind to the same epitope as dom1h-131-206.

The HEK293 cell line is used for transient expression, and, following the in vitro evaluation of the expressed antagonists and the identification of molecules with the desired properties, such as high affinity for TNFR1 (e.g., K_{d}<50 nM, or <10 nM, or < or 5 nM), and potent inhibition of TNFR1 signaling (e.g., IC₅₀<50 nM, or <10 nM, or <5 nM). Generally, it is picomolar (pM) affinity, such as around or about 19 pM affinity or 20 pM, 15 pM, 10 pM, 5 pM, 2 pM, or 1 pM affinity.

Stable cell lines are prepared in a derivative of CHO cells. Transient expression is optimized in CHO DG44 cells (e.g., CHO-DG44 (DHFR-) and FreeStyle™ CHO-S cells, Invitrogen), resulting in transfected pools that are screened to identify or select high expressing clones. No poly-histidine, or other purification tags, are used. Instead, proteins for screening are purified from serum-free medium by HPLC in combination with other well-known methods. The matrix used for HPLC is the Amsphere™ A3 Protein A chromatography resin (JSR Life Sciences), or other similar resin, following the manufacturer's protocols. If the protein is not at least 95% pure, as judged by size exclusion HPLC, further purification is performed (e.g., ion exchange or hydrophobic chromatography).

Endotoxin is removed (see, e.g., Vazquez-Lombardi et al. (2018) for an exemplary protocol). After protein purification, endotoxin levels are determined using a detection kit, such as the QCL-1000 Endpoint Chromogenic LAL Assay Kit (Lonza). For endotoxin removal, the theoretical pI of the purified protein is determined using a sequence analysis tool (e.g., ExPASy ProtParam), and the pH low-endotoxin PBS buffer is adjusted to a pH that is below, but close to, the theoretical pI of the purified protein. The protein sample then is dialyzed against at least 30 volumes of pH-adjusted PBS for at least 2 hours at 4° C. An additional dialysis step is performed overnight, and then again for at least 2 hours the following day. The sample then is purified using anion exchange affinity chromatography, retested to determine endotoxin levels, and the process is repeated until an acceptable level of endotoxin is achieved. The size and purity of the protein products is determined by SDS-PAGE analysis or other suitable method. Alternatively, other methods can be used, such as the Proteus Endotoxin Removal Kit, and accompanying manufacturer's handbook (BIORAD, see, bio-rad-antibodies.com/static/uploads/ifu/pur030.pdf). This step can be repeated until endotoxin reaches desirable levels, typically >0.5 endotoxin units/ml (less than or equal to 0.5 endotoxin units/ml).

### Screening of Purified Proteins

The purified TNFR1 antagonist molecule candidates are screened to measure binding affinity for the extracellular domain of TNFR1, using methods described above in the detailed description, or methods that are known in the art, such as, for example, immunoassays (e.g., ELISA), surface plasmon resonance (SPR), isothermal titration calorimetry (ITC), or other kinetic interaction assays known in the art. SPR can be performed using several commercially available platforms, such as the BIAcore systems (GE Healthcare Life Sciences), for example. Exemplary assays are described, for example, in Lang et al. (2015) J. Biol Chem 291:5022-5037, which describes and compares the various assays to assess binding affinity. Candidates that are selected include those that have a K_{d} value of <5 nM.

The TNFR1 antagonists also are screened to determine whether binding to TNFR1 is competitive or non-competitive with respect to TNF, using methods known in the art, such as SPR. If an inhibitor binds to a receptor (e.g., TNFR1) and blocks binding of the ligand (e.g., TNF), for example, by attaching to the active site, this is competitive inhibition, because the inhibitor “competes” with the substrate for the enzyme; that is, only the inhibitor or the substrate can be bound at a given moment. In non-competitive inhibition, the inhibitor does not block the ligand from binding to the ligand-binding site on the receptor. Instead, it attaches at another site and blocks the receptor from responding to bound ligand. This inhibition is said to be “non-competitive” because the inhibitor and substrate can both be bound at the same time. Thus, if ligand is added to the receptor-binding assay at a saturating concentration and it does not inhibit binding of the antibody, then the two molecules are independent and non-competitive. The reverse is also valid. If the two are competitive, then increasing concentrations of the antibody will prevent TNF from binding to the receptor. This is valid whether the binding assays are done on cells, or with receptors or ligands bound to a surface. For exemplary assays, see, e.g., Frey et al. (2001) Current Protocols in Neuroscience, “Receptor Binding Techniques,” available at doi.org/10.1002/0471142301.ns0104s00.

For example, the ability for TNF to bind human TNFR1 coated on a BIAcore chip first is determined. The TNFR1 surface then is saturated with the TNFR1 antagonist molecule, and TNF subsequently is injected, and the binding of TNF to TNFR1 is re-evaluated. If the binding is non-competitive, they both bind to TNFR1; if it is competitive, they will interfere with each other. If the binding of TNF is unaffected, or only slightly reduced, then binding of the antagonist to TNFR1 is non-competitive with respect to TNF, and if the binding of TNF is abrogated, or significantly reduced, then the binding of the antagonist is considered to be competitive with respect to TNF. For purposes herein, competitive binders are selected.

The TNFR1 antagonist molecules are further screened to determine their capacity to inhibit TNFR1 signaling in the presence of TNF on cells, using methods known in the art, such as, for example, the method described by McFarlane et al. (2002) FEBS Lett. 515(1-3):119-126, which results in the activation of NFxB-luciferase expression (i.e., a gene reporter assay). Cells used in these experiments do not express TNFR1 or TNFR2 (e.g., the myeloma cell lines AMO1, U266, and L363; see, e.g., Rauert et al. (2011) Cell Death Dis. 2(8):e194), unless transfected with a TNFR1- or TNFR2-expressing plasmid. Alternatively, human cell lines in which TNFR1 and/or TNFR2 genes are inactivated or knocked out using CRISPR vectors, antisense RNA expression, or other methods known in the art, can be used. TNFR1⁻ and/or TNFR2⁻ cell lines, which can be obtained from commercial sources (e.g., Genoway and Synthego), also can be used. These cell lines can then be specifically transfected with TNFR1 and/or TNFR2 expression cassettes. For example, cells expressing TNFR1, TNFR2, or TNFR1 and TNFR2, can be used to assess the selectivity of the antagonists for TNFR1, and to determine the potency of inhibition of TNF signaling via TNFR1.

To determine the inhibition of TNFR1 signaling by the TNFR1 antagonists, cells expressing human TNFR1 are transiently transfected with a NF-κB-luciferase reporter construct using Lipofectamine, and receptor-stimulated luciferase transcription is measured 48 hours after transfection. Cells stably expressing TNFR1 and NF-κB-luciferase are plated into 24-well plates at a density of 1×10⁵ cells/ml culture media, and incubated until they reach 80% confluency (~24 hours). The cells then are incubated with 50 ng/ml TNF and varying concentrations of TNFR1 antagonist for 6 hours. NF-κB-stimulated luciferase activity is detected by washing the cells twice with ice-cold PBS, adding 200 μl of ice-cold lysis buffer (25 mM Tris-phosphate pH 7.8, 8 mM MgCl₂, 1 mM DTT, 1% Triton X-100, 15% glycerol), and incubating on ice for 5 min. Cell extracts then are scraped into 1.5 ml Eppendorf tubes, centrifuged to pellet cell debris, and 100 μl of the supernatant is used to measure luciferase induction using a luminometer. The IC₅₀ then is calculated by plotting the relative luminescence units (RLUs) against TNFR1 antagonist concentration, and using a curve fitting software, such as GraphPad Prism. Other similar assays for evaluating the inhibition of TNFR1 signaling include assays that measure the induction of phospho-IκBα, which is indicative of the activation of the classical NF-κB pathway, in cells expressing TNFR1 that are treated with TNF, (see, e.g., Rauert et al. (2011) Cell Death Dis. 2(8):e194).

TNFR1 antagonist candidates that exhibit inhibition of TNFR1 signaling of at least 80%, and IC₅₀ values approximately equal to the K_{d} (i.e., <5 nM), as determined above, are selected for further optimization.

### Example 2

### Optimization of Selected Candidate Univalent TNFR1 Antagonist Molecules Optimization of Affinity for TNFR1 and Potency of TNFR1 Signaling Inhibition

Candidate TNFR1 antagonist molecules, which meet the selection criteria outlined above (i.e., high affinity for TNFR1 and potent inhibition of TNFR1 signaling, with K_{d} and IC₅₀ values of <5 nM), are optimized to increase affinity for TNFR1, and potency of TNFR1 signaling inhibition. For purposes herein the candidates also are selected so that they do not have TNFR1 agonist activity.

These parameters are achieved by methods that include one or more of random mutagenesis, site-directed mutagenesis, molecular modeling, and/or error-prone PCR to achieve K_{d} and IC₅₀ values of as low as <1 nM, generally, at least equal to or <100 nM, <50 nM, <10 nM, < or 5 nM. For example, this can be achieved by the method (see, Tiller et al. (2017) Frontiers Immunol. 8:986) in which amino acids most critical to binding are conserved, while the remaining amino acids of the V_{H} domain are subjected to mutagenesis, and a phage library is prepared, and screened for high affinity binding variants for TNFR1, which are selected. In accord with this method, a phage display library to select such variants is produced. In a first step, computational and experimental alanine scanning mutagenesis identifies sites in the complementarity-determining regions (CDRs) that are permissive to mutagenesis while maintaining antigen binding. Next, the most permissive CDR positions are mutated using degenerate codons to encode wild-type residues, and a small number of the most frequently occurring residues at each CDR position, based on natural antibody diversity. This mutagenesis approach results in antibody libraries with variants that have a wide range of numbers of CDR mutations, including antibody domains with single mutations, and others with tens of mutations. In a last step, the libraries (~10 million variants) displayed on the surface of yeast are sorted to identify CDR mutations with the greatest increases in affinity. Lastly, the candidates are those that do not have TNFR1 agonist activity; they can be selected by appropriate assays (i.e., THP1 assay), and/or by identifying those that bind to the same epitope as the dAb of SEQ ID NO:59, or the construct designated EN-1206, exemplified in the Examples below.

### Half-Life Extension

Optimized molecules then are linked to a half-life extending moiety, for example, by fusion with IgG Fc domains, particularly modified Fc domains, or human serum albumin (HSA), or by PEGylation, as described above in the detailed description, to achieve an in vivo serum half-life of about 1, 2, 3, 4, 5, 6, 7, 8, 9, 10, or more, days, such as 10-12 days, in SCID mice. The modified molecules then are retested in the in vitro assays described above to ensure that the high affinity binding to TNFR1 and potent inhibition of TNFR1 signaling is retained. Production of successful candidates is scaled-up for further in vitro and in vivo assays.

### In Vitro Assays

### Phagocytosis Assays

As discussed and described in the detailed description, existing TNF blockers, which inhibit TNFR1 activity, also inhibit TNFR2. TNF blockers have two types of black box warnings from regulatory agencies. The first is susceptibility of anti-TNF (TNF blocker) treated patients to infection. These infections can involve various organ systems and sites due to bacterial, mycobacterial (e.g., tuberculosis), fungal (e.g., histoplasmosis, aspergillosis, candidiasis, coccidioidomycosis, blastomycosis, and pneumocystis), viral (e.g., hepatitis B), and other opportunistic pathogens (organisms that usually do not cause disease in healthy people, but can cause serious illness when a person's immune system (resistance) has weakened). Another issued black box warning is related to pediatric malignancy (see, e.g., online.epocrates.com/u/10b3301/Humira/Black+Box+Warnings). As discussed in the detailed description, this vulnerability results from the total blockade of signaling by TNFR1 and TNFR2 when their ligand, TNF, is blocked. This suppression of innate immunity effect of TNF is mediated by TNFR2 (see, e.g., Ahmad et al. (2018) Front. Immunol. 9:2572), and is primarily mediated by the transmembrane form of TNF, which preferentially activates TNFR2 (see, e.g., Miller et al. (2015) Journal of Immunology 195(6):2633-2647).

It has been shown that anti-TNF therapeutics, such as adalimumab, infliximab, and etanercept, have an inhibitory effect on IFN-γ-induced phagosome maturation in phorbol myristate acetate-differentiated human THP-1 cells. Adalimumab and infliximab, but not etanercept, suppress phagosome maturation in primary human peripheral blood monocyte-derived macrophages in the presence or absence of IFN-7 (see, e.g., Harris et al. (2008) J. Infect. Dis. 198:1842-1850). In view of the above, an advantage of a specific TNFR1 inhibitor construct is preservation of TNFR2 function, and therefore, macrophage function; macrophages are important in ridding an organism of opportunistic infection. Opportunistic infectious agents include mycobacteria that cause tuberculosis (see, e.g., Fraga et al. (2018) Curr. Issues Mol. Biol. 25:169-198). Macrophage function is disrupted in autoimmune disease in patients treated with TNF blockers. A TNFR1-specific antagonist only inhibits TNFR1 function, thereby avoiding disruption of TNFR2 function, which is needed for normal macrophage function. These TNFR1-specific antagonists can be identified by an assay for anti-TNF effects on macrophage phagocytosis. Among the TNFR1-specific antagonists that are identified, are those that also stimulate TNFR2 function, thus boosting macrophage activity (TNFR1 antagonist and TNFR2 agonist).

Assay to Determine Effects of TNFR1 Antagonists on Macrophage Responses to Mycobacterium tuberculosis Infection

TNF plays an important role in mediating the inflammatory host response to various pathogens, including Mycobacterium tuberculosis; TNF also plays a role in the immunopathology of tuberculosis (TB). Mycobacterial infection induces the secretion of TNF by macrophages. TNF enhances the ability of macrophages to phagocytose and kill mycobacteria. TNF also stimulates macrophage apoptosis, leading to increased killing and presentation of mycobacterial antigens by dendritic cells. TNF also is required for the formation and maintenance of granulomas; neutralization of TNF in mice chronically infected with M. tuberculosis disrupts the integrity of granulomas, worsening infection and increasing mortality. TNF blockers, such as adalimumab and infliximab, increase the susceptibility to infection with various pathogens, including M. tuberculosis, and increase the risk of reactivation of latent tuberculosis, by suppressing mycobacteria-containing phagosome maturation in human macrophages. Phagosome maturation (i.e., phagosome acidification and fusion with lysosomes) is essential for the presentation of mycobacterial antigens to T-cells, and the initiation of adaptive immune responses (see, e.g., Harris et al. (2008) J. Infect. Dis. 198:1842-1850).

In order to identify and/or characterize TNFR1 antagonists, the effects of TNFR1 antagonists provided herein on macrophage responses to infection with M. tuberculosis is assessed. Mycobacteria-containing phagosome maturation in human macrophages is analyzed, and compared to phagosome maturation in TNF blockers, such as adalimumab, using the methods described in Harris et al. (2008) J. Infect. Dis. 198:1842-1850. The TNFR1 antagonists that do not increase susceptibility to infection compared to a known TNF blocker, such as adalimumab, are of interest.

### Preparation of THP-1 Cells and Monocyte-Derived Macrophages (MDMs)

Human THP-1 cells are cultured in RPMI 1640 (Invitrogen) with 10% fetal bovine serum (FBS; Gibco). The cells are differentiated into macrophage-like cells by treatment with 100 nmol/L phorbol myristate acetate (PMA) for 24 hours, and then cultured in normal medium for 3 days. To prepare human monocyte-derived macrophages (MDMs), peripheral blood mononuclear cells (PBMCs) are isolated from the blood of healthy donors using density gradient centrifugation on Histopaque®-1077 (Sigma). Monocytes are isolated by adherence to gelatin-coated culture dishes, and cultured overnight in RPMI 1640 with 5% human AB serum (Sigma). Adherent cells are removed with 10 mmol/L EDTA in PBS, and grown on coverslips in 12-well plates for 10 days. THP-1 cells and MDMs are grown on coverslips at a concentration of 2×10⁵ cells/well.

### Preparation of the Mycobacteria

Green fluorescent protein (GFP)-labeled Mycobacterium bovis bacillus Calmetter-Guerin (GFP-BCG), and the attenuated M. tuberculosis strain H37Ra and its virulent counterpart, H37Rv, are grown in Middlebrook 7H9 broth with 0.5% Tween, 0.2% glycerol, and 10% albumin-dextrose-catalase supplement (BD). The mycobacteria are grown to log phase before use, and are resuspended in RPMI 1640 with 10% FBS before infection. M. tuberculosis strain H37Ra is fluorescently labeled with PKH67 (Sigma), and strain H37Rv is labeled with fluorescein isothiocyanate (FITC, 1 mg/mL; Sigma), following the manufacturer's protocols.

### Determination of Phagosome Maturation

Mycobacteria can inhibit the fusion of phagosomes with lysosomes, preventing the acidification and recruitment of lysosomal hydrolases. This blockade of phagosome maturation can be overcome by pre-treating the macrophages with IFN-7. Treatment of cells infected with mycobacteria with TNF (5 ng/mL) also enhances phagosome acidification. To determine the effects of the TNFR1 antagonist and TNF blockers on the fusion of mycobacterial phagosomes with lysosomes in macrophages, PMA-differentiated THP-1 cells or human peripheral blood MDMs are infected with GFP-BCG, or PKH67-labeled M. tuberculosis strain H37Ra, or FITC-labeled M. tuberculosis strain H37Rv, in the presence of TNFR1 antagonist or TNF blocker, with or without IFN-7 treatment, and phagosome-lysosome fusion is analyzed by confocal microscopy, using LysoTracker® Red (LT) as a marker for acidified phagosomes, and CD63 and cathepsin D as phagolysosomal markers. The inhibition of IFN-γ-induced phagosome maturation/acidification is determined by colocalization of labeled mycobacteria with LysoTracker® Red, CD63 or cathepsin D. For example, a decrease in the percent colocalization of labeled mycobacteria with LT, or with CD63, or with cathepsin D, as compared to the control, is indicative of the inhibition of IFN-γ-induced phagosome acidification.

TNFR1 antagonist or TNF blocker (e.g., adalimumab, infliximab, etanercept, or others; 10 μg/mL), with or without IFN-γ (200 U/mL), are added to the THP-1 cells or MDMs for 24 hours before infection. As a control, cells are treated with medium only, or with 10 μg/mL human IgG1 from patients with myelomas producing IgG1 (Calbiochem). Cells then are infected with M. bovis GFP-BCG, PKH67-labeled M. tuberculosis H37Ra, or FITC-labeled M. tuberculosis H37Rv for 15 min, washed 3 times with PBS to remove unbound mycobacteria, and incubated for 2 hours. The multiplicity of infection (MOI) is recorded microscopically 15 min after infection of macrophages by acid-fast bacilli staining. Cells are infected at an MOI of 1-5 bacilli in approximately 70% of cells.

After the 2 hour incubation, cells are fixed in 2% paraformaldehyde for 20 min at room temperature (RT); for strain H37Rv, cells are fixed in 4% paraformaldehyde overnight. Cells are then permeabilized with 0.1% Triton X-100 in PBS, and blocked with 1% bovine serum albumin and 1% goat serum in PBS for 30 min at RT. Cells are incubated with primary antibody (1 μg/mL mouse monoclonal antibody against CD63 (LAMP-3; Santa Cruz Biotechnology); or 10 μg/mL mouse monoclonal antibody against cathepsin D (Calbiochem)) for 1 hour at RT, followed by secondary antibody (4 μg/mL Alexa Fluor 488- or 568-labeled goat anti-mouse IgG; Invitrogen) for 1 hour at RT. Alternatively, prior to fixation, the cells are incubated with LysoTracker® Red DND-99 (100 nmol/L; Invitrogen) for the final 60 min of incubation with the mycobacteria. LysoTracker® Red DND-99 is a red-fluorescent dye for labeling and tracking acidic organelles (such as acidified phagosomes) in live cells.

The coverslips are mounted onto glass slides with fluorescent mounting medium (Dako), and images are recorded on a laser scanning confocal microscope, such as the Olympus FluoView™ 1000 and Zeiss LSM 510 laser scanning confocal microscope. Images are analyzed and prepared using the appropriate software and Adobe Photoshop.

### Measurement of TNF

THP-1 cells are prepared as described above and infected with BCG or M. tuberculosis H37Ra, with or without IFN-γ pretreatment. The levels of immunoreactive TNF in supernatants (secreted in response to mycobacterial infection) are measured using a commercial ELISA kit (R&D systems), in accordance with the manufacturer's instructions.
Inflammatory Cytokines are Up-regulated in TNF Blockade vs. Specific TNFR1 Inhibition in Collagen-Induced Arthritis

Transgenic mice (C57/BL6N.Q;H-2q/HuTNFR1/Hunt) with collagen-induced arthritis (CIA) (see, e.g., McCann et al. (2014) Arthritis & Rheumatology 66(10):2728-2738) are treated with TNF blocker and the TNFR1-specific antagonist. Serum inflammatory cytokines are evaluated (IFN-gamma, IL-12p70, IL-10, RANTES (CCL5); see, e.g., McCann et al. (2014) Arthritis & Rheumatology 66(10):2728-2738). Antagonists that are specific for blocking TNFR1 induce significantly less of one or more of IFN-gamma, IL-12p70, IL-10, or RANTES (CCL5). This results from sparing TNFR2 function and regulatory T-cell function in the spleen and lymph nodes.

### In Vivo Assays

Studies are performed with humanized (HuTNFR1/HuTNF) transgenic mice. The TNFR1 antagonist molecules are assessed for efficacy in multiple autoimmune disease models. These include the models described above, which express human transgenes for TNFR1 and TNF. Alternative models for autoimmune disease are known. For example, models, including those for RA, are described in detail in Schinnerling et al. ((2019) Front. Immunol 10:203). To establish efficacy, the specific TNFR1 antagonist constructs, as well as other constructs provided herein, are tested in more than one model. Included among the models are at least rheumatoid arthritis (RA), Crohn's disease, and multiple sclerosis (experimental autoimmune encephalitis) models (discussed in the detailed description).

Inflammatory bowel diseases (IBDs, including ulcerative colitis and Crohn's disease) were the first approved indication for TNF Blockers. Numerous mouse models of these diseases are available and have been described by Mueller ((2002) Immunology 105(1):1-8). As discussed in the detailed description, autoimmune neurodegenerative diseases, including multiple sclerosis (MS) and Alzheimer's disease, are important disease targets. Alzheimer's disease (AD) is the leading cause of dementia worldwide, and represents one of the most serious health issues for the elderly. An estimated 5.4 million Americans have AD, and this number is expected to triple by 2050 if there are no medical breakthroughs to stop, prevent, or slow the disease (see, e.g., Chang et al. (2017) J Cent. Nerv. Syst. Dis. 9:1179573517709278). Evidence indicates that TNFR1 antagonist constructs and other constructs provided herein are therapeutic candidates, since subjects who have experienced prolonged treatment with TNF blockers are less likely to develop the disease (see, e.g., Chou et al. (2016) CNS Drugs 30:1111). Data show that up-regulated TNF expression is associated with different neurodegenerative diseases and conditions, such as Alzheimer's disease, Parkinson's disease, stroke, and multiple sclerosis (see, e.g., McCoy et al. (2008) J. Neuroinflammation 5(1):45). Existing TNF blockers, however, do not appear to be effective for treating, ameliorating, preventing, or slowing disease progression (see, e.g., Tortarolo et al. (2015) J. Neurochem. 135:109-124). As described herein, various lines of evidence indicate that TNF blockers do not work in such indications because of co-inhibition of TNFR1 and TNFR2; TNFR2 has neuro-protective properties that are lost by treatment with TNF blockers. Others have attempted to approach this problem with various forms of “TNFR1 inhibitors” or “TNFR2 agonists.” None of these studies included cross-reactivity to determine whether TNFR1 or TNFR2 was selectively targeted, as opposed to being one of many epitopes in the body that could be targeted.

This problem is solved herein. First, a family of anti-TNFR1 antagonists is generated, and tested in the above models, showing that they act as predicted. Then, immunochemistry is used to demonstrate that they are selective. Several contract research laboratories offer this service (e.g., Sino Biological, Inc., and LSBio).

As discussed in the detailed description, other autoimmune and chronic inflammatory disease states are associated with the presence of TNF. These include type II diabetes and endometriosis. Mouse models of these diseases are known, and HuTNFR1/HuTNF transgenic versions of these mice are used to demonstrate efficacy with the specific anti-TNFR1 antagonists provided herein.

### Acute Respiratory Distress Syndrome

Viral pathogens of the respiratory tract (e.g., influenza, SARS viruses/coronaviruses) infect respiratory epithelial cells, and tissue-resident alveolar macrophages are the first responders to viral infection in the lung. They effect clearance through the phagocytosis of opsonized viral particles or infected apoptotic cells and the release of a plethora of inflammatory cytokines and chemokines to initiate an immune response (see, e.g., Herold et al. (2015) Eur. Resp. J. 45:1463-1478). TNF blockers have been shown to extend survival of mice infected with influenza (see, e.g., Shi et al. (2013) Crit. Care 17:R301). TNF blockers have been used for treating SARS-Cov 2 (see, e.g., Feldmann et al. (2020) Lancet 395:1407-1409). As described in the detailed description, a known effect of TNF blockers is to reduce regulatory T-cells (Tregs), which is problematic since Tregs are a natural suppressor of inflammation. Thus, a TNFR1-specific inhibitor, that does not interact with TNFR2 or that agonizes TNFR2, as described and provided herein, is superior for this purpose. To test the constructs provided herein, mice are engineered to lack endogenous TNFR1, and to express HuTNFR1/HuTNF. These mice will manifest the acute respiratory distress syndrome induced by HuTNF/HuTNFR1 following infection with influenza (as described in Shi et al. (2013) Crit. Care 17:R301). TNFR1-specific antagonist constructs that do not antagonize TNFR2 are administered. Efficacy is determined by any of several criteria:
1. Significantly decreased circulating inflammatory cytokines (e.g., IFN-gamma, IL-1alpha, IL1-beta, and IL-17) vs. a TNF blocker;
2. Significantly faster recovery measured by weight gain (see, e.g., Shi et al. (2013) Crit. Care 17:R301), compared to a TNF blocker; and
3. Significantly increased survival (see, e.g., Shi et al. (2013) Crit. Care 17:R301), compared to a TNF blocker.

Transgenic mice that express human TNFR1 and human TNFR2 for use in disease models are generated by standard genetic engineering methods that are known in the art, such as those described by Atretkhany et al. (2018) Proc. Natl. Acad. Sci. U.S.A. 115(51):13051-13056. Particular in vivo assays for various diseases and conditions are as follows. For example, humanized RA mouse models, such as the collagen-induced arthritis (CIA) model of RA, or any other animal models of RA that are known in the art and/or described herein, are used to assess the therapeutic effects of the TNFR1 antagonist molecules provided herein, and compare them to the therapeutic effects of anti-TNF therapies, such as etanercept or adalimumab. The TNFR1 antagonist molecules provided herein, or an anti-TNF therapy, such as etanercept or adalimumab, is administered to the animal daily for a total of 10 days following the onset of clinical arthritis in one or more limbs. The degree of swelling in the initially affected joints is monitored by measuring paw thickness using calipers. Serum is drawn from mice for the measurement of proinflammatory cytokines and chemokines, such as, for example, granulocyte-macrophage colony stimulating factor (GM-CSF), interleukin-10 (IL-10), IL-1β, IL-6, IL-8, RANTES (CCL5) and monocyte chemoattractant protein 1 (MCP-1; also known as CCL2). The regression of RA in the mouse models then is compared between the mice administered the TNFR1 antagonist molecule, and the mice administered etanercept or adalimumab.

The TNFR1 antagonist molecules provided herein also are tested in humanized mouse models of severe acute respiratory syndrome (SARS), and of viral-induced cytokine storm. Mouse models of SARS are generated, for example, by infecting humanized hTNFR1 (or hTNFR1/hTNFR2) knock-in mice with varying doses, such as 10², 10³, 10⁴, and 10⁵ plaque-forming units (PFUs), of SARS-CoV. The TNFR1 antagonist molecule is administered to the infected mice, and survival is evaluated, and compared to survival of mice administered an anti-TNF therapy, such as adalimumab.

Viral-induced mouse models of cytokine storm include, for example, lymphocytic choriomeningitis virus (LCMV)-induced models of cytokine storm syndrome (CSS). LCMV-induced mouse models of CSS are generated by administering 2×10⁵ PFUs of LCMV-Armstrong, intraperitoneally to 8-12 week-old Perforin-deficient (Prf^{-/-}) or Prf-Tmem178 double knockout mice. To deplete monocytes/macrophages, 100 μl of clodronate-liposomes are intravenously injected into Prf^{-/-} mice two days prior to the LCMV infection, and 48 and 96 hours later. Alternatively, 1 mg of the neutralizing anti-CSF1 antibody (Clone 5A1, BioXCell) is administered 2 days prior to infection, and 0.5 mg antibody is administered 48 and 96 hours later. Animals are bled via submandibular vein puncture to measure serum cytokines on days 3 and 8 post-infection (see, e.g., Mahajan et al. (2019) J. Autoimmun. 100:62-74).

### Example 3

Exemplary TNFR1 Antagonist Constructs that Contain Human TNFR1 Antagonist Antibody Fragments (dAbs, scFvs, Fabs)

Provided herein is a TNFR1 antagonist construct that selectively inhibits TNFR1, without inhibiting TNFR2. To avoid TNFR1 receptor clustering, which agonizes TNFR1, the TNFR1 antagonist is monomeric and monovalent. It is shown herein, however, that TNFR1 antagonists can be bivalent. As discussed, these include TNFR1 agonists that bind the same epitope as the construct designed EN-1206.

For monovalent constructions, the TNFR1 antagonist contains a human single domain antibody (dAb) that is specific for TNFR1. The dAb contains a variable region heavy chain (V_{H}) or a variable region light chain (V_{L}) domain. For example, the dAb contains any of the dAbs whose amino acid sequences are set forth in any one of SEQ ID NOs: 54-672, or a dAb with at least or at least about 90% or 95% sequence identity to the dAb of any of SEQ ID NOs: 54-672, that retains binding affinity for TNFR1. Alternatively, the TNFR1 antagonist contains an scFv, a Fab, or other antigen-binding fragment, such as those derived from a human TNFR1 antagonist antibody, such as H398 or ATROSAB. For example, the TNFR1 antagonist contains the H398-derived scFv set forth in SEQ ID NO: 677 or 678; or the ATROSAB-derived scFvs set forth in any one of SEQ ID NOs: 673-676; or the ATROSAB-derived Fab fragment light and heavy chains set forth in any of SEQ ID NOs: 679 and 680, respectively (FabATR), or SEQ ID NOs: 681 or 682, respectively (Fab 13.7); or an scFv or Fab fragment with at least or at least about 90% or 95% sequence identity to the scFvs of any of SEQ ID NOs: 673-678, or the Fab light and heavy chains of any of sequence ID NOs: 679 and 680, respectively (FabATR), or SEQ ID NOs: 681 or 682, respectively (Fab 13.7), and that retain the affinity for TNFR1.

As described herein, the dAb is selected so that it does not agonize TNFR1 or is identified as one that binds the same epitope as the construct designated EN-1206.

The TNFR1 antagonist is fused to a serum half-life extender, such as an IgG Fe, particularly an Fc modified to eliminate or reduce ADCC, ADCP, and/or CDC, human serum albumin (HSA), and/or a poly(ethylene)glycol (PEG) molecule. For example, the C-terminus of the human anti-TNFR1 dAb, scFV, Fab or other antigen-binding fragment, is fused with the N-terminus of the Fc region of a human IgG1 or IgG4 antibody via a linker. An IgG1 Fc region, such as the IgG1 Fc derived from trastuzumab (see, SEQ ID NO:27), or an IgG4 Fc region, such as the IgG4 Fc derived from nivolumab (see, SEQ ID NO:30), is used. The linker includes a portion of the hinge sequence of trastuzumab, containing the sequence of amino acid residues SCDKTH (corresponding to residues 222-227 of SEQ ID NO:26), when the Fc is derived from trastuzumab, or can contain the hinge sequence of nivolumab, containing the sequence of amino acid residues ESKYGPPCPPCP (corresponding to residues 212-223 of SEQ ID NO:29), or a portion thereof that provides flexibility or other structural property, when the Fc is derived from nivolumab. To confer protease resistance and to increase the flexibility of the fusion protein, the SCDKTH or ESKYGPPCPPCP hinge sequence is replaced with a short glycine-serine (GS) peptide linker, such as, for example, (GSGS) or (GGGGS)ₙ (see, e.g., residues 199-202, and 116-120 of SEQ ID NO:707, respectively), where n=1-5 or 1-6, or other combination of Gly and Ser residues, such as, for example, GGGGSGGGGSGGGGS (e.g. residues 116-130 or SEQ ID NO:707). In other embodiments, the C-terminus of the human anti-TNFR1 dAb, scFv, Fab or other antigen-binding fragment, is linked to the GS linker, and the GS linker is connected to all or a portion, sufficient to provide flexibility, of the trastuzumab or nivolumab hinge sequence, which is connected to the N-terminus of the corresponding Fc region. In some embodiments, a second Fc subunit, is linked to the first Fc subunit, to increase the serum half-life and stability of the molecule. Because there are two Fc regions, any resulting construct is not a fusion protein since it contains one non-contiguous Fc region. In some embodiments, the N-terminus of the human TNFR1 antagonistic dAb, scFV, Fab or other antigen-binding fragment, is fused to the C-terminus of the serum half-life extender via a linker, as described above.

Also provided herein are TNFR1 antagonist fusion proteins that contain an anti-TNFR1 dAb, scFv, Fab, or other antigen-binding fragment, fused with human serum albumin (HSA), via a short peptide linker, such as (GSGS)ₙ or (GGGGS)ₙ, where n=1-5 or 6, such as, for example, GGGGSGGGGSGGGGS.

Also provided herein are TNFR1 antagonist molecules that contain an anti-TNFR1 dAb, scFv, Fab, or other antigen-binding fragment, linked to a PEG molecule that is at least 30 kDa in size.

As described herein, these constructs can be modified to reduce or eliminate immunogenicity. The TNFR1 antagonist dAb, scFv, Fab, or other antigen-binding fragment is analyzed by in silico, in vitro and/or in vivo methods to predict or identify immunogenic sequences. Upon identification of immunogenic sequences, such as B-cell and/or T-cell epitopes, the identified sequences are modified by mutagenesis, for example, by alanine scanning, as described elsewhere herein, to de-immunize the epitopes/remove or replace the immunogenic sequences.

The following are exemplary constructs of the TNFR1 antagonist fusion proteins described and provided herein. In all embodiments that include the Fc of trastuzumab or the Fc of nivolumab, the Fc regions optionally are modified to reduce or eliminate immune effector functions, including ADCC, ADCP, and CDC, and also, optionally are modified to enhance binding to FcRn, increasing the serum half-life of the fusion proteins, and also to optionally replace or otherwise modify or remove immunogenic sequences.

Fc modifications that reduce or eliminate immune effector functions are summarized in Table 9, above, and Fc modifications that enhance FcRn binding are summarized in Table 7, above. Any one or a combination of such modifications is/are included in the Fc regions of the fusion proteins provided herein. All of the exemplary constructs provided herein also are prepared with the TNFR1 antagonist at the C terminus of the fusion protein, instead of at the N-terminus.

1s) DOM1 h-574-208-GGGGSGGGGSGGGGS-BSA

Provided herein is a TNFR1 antagonist fusion protein, containing the human TNFR1 antagonist dAb DOM1 h-574-208 (SEQ ID NO:54). The C-terminus of DOM1 h-574-208 is fused to a GGGGSGGGGSGGGGS peptide linker, which is fused to the N-terminus of human serum albumin (HSA) without the signal peptide (corresponding to residues 19-609 of SEQ ID NO:35). The DOM1 h-574-208-GGGGSGGGGSGGGGS-HSA fusion protein has the following sequence (SEQ ID NO:721):

EVQLLESGGGLVQPGGSLRLSCAASGFTFDKYSMGWVRQAPGKGLEWVSQ
ISDTADRTYYAHAVKGRFTISRDNSKNTLYLQMNSLRAEDTAVYYCAIYT
GRWVPFEYWGQGTLVTVSSGGGGSGGGGSGGGGSRGVFRRDAHKSEVAHR
FKDLGEENFKALVLIAFAQYLQQCPFEDHVKLVNEVTEFAKTCVADESAE
NCDKSLHTLFGDKLCTVATLRETYGEMADCCAKQEPERNECFLQHKDDNP
NLPRLVRPEVDVMCTAFHDNEETFLKKYLYEIARRHPYFYAPELLFFAKR
YKAAFTECCQAADKAACLLPKLDELRDEGKASSAKQRLKCASLQKFGERA
FKAWAVARLSQRFPKAEFAEVSKLVTDLTKVHTECCHGDLLECADDRADL
AKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKC
CAAADPHECYAKVFDEFKPLVEEPQNLIKQNCELFEQLGEYKFQNALLVR
YTKKVPQVSTPTLVEVSRNLGKVGSKCCKHPEAKRMPCAEDYLSVVLNQL
CVLHEKTPVSDRVTKCCTESLVNRRPCFSALEVDETYVPKEFNAETFTFH
ADICTLSEKERQIKKQTALVELVKHKPKATKEQLKAVMDDFAAFVEKCCK
ADDKETCFAEEGKKLVAASQAALGL

Alternatively, the GGGGSGGGGSGGGGS linker is replaced by a Gly-Ser linker, such as a (GSGS)ₙ or a (GGGGS)ₙ linker, where n=1, 2, 3, 4 or 5, or any other suitable short peptide linker as described herein or as known in the art.

1t) DOM1 h-574-208-GGGGSGGGGSGGGGS-PEG_{30kDa}

Provided herein is a TNFR1 antagonist fusion protein, containing the human TNFR1 antagonist dAb DOM1 h-574-208 (SEQ ID NO:54). The C-terminus of DOM1 h-574-208 is fused to a GGGGSGGGGSGGGGS peptide linker, which is covalently linked to a PEG molecule of 30 kDa in size.

Alternatively, the GGGGSGGGGSGGGGS linker is replaced by a Gly-Ser linker, such as a (GSGS)ₙ or a (GGGGS)ₙ linker, where n=1, 2, 3, 4 or 5, or any other suitable short peptide linker as described herein or as known in the art. Alternatively or additionally, the PEG molecule can have a molecular weight of at least or more than 30 kDa, such as, for example, 35 kDa, 40 kDa, 45 kDa, or 50 kDa.

1u) DOM1 h-131-206-SCDKTH-Trastuzumab Fc

Provided herein is a TNFR1 antagonist fusion protein, containing the human TNFR1 antagonist dAb DOM1 h-131-206 (SEQ ID NO:59). The C-terminus of DOM1 h-131-206 is fused to a portion of the hinge sequence of trastuzumab, containing at least the sequence of residues SCDKTH (corresponding to residues 222-227 of SEQ ID NO:26), which is fused to the N-terminus of the trastuzumab Fc region (corresponding to residues 234-450 of SEQ ID NO:26; see, also, SEQ ID NO:27). The DOM1 h-131-206-SCDKTH-Trastuzumab Fc fusion protein has the following sequence (SEQ ID NO:722):

EVQLLESGGGLVQPGGSLRLSCAASGFTFAHETMVWVRQAPGKGLEWVSH
IPPDGQDPFYADSVKGRFTISRDNSKNTLYLQMNSLRAEDTAVYHCALLP
KRGPWFDYWGQGTLVTVSSSCDKTHAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFF
LYSKLTVDKSRWQQGNVFSCSVMHEALHNHYTQKSLSLSPGK

Alternatively, the SCDKTH hinge sequence is replaced by a portion containing at least 5, 6, 7, 8, 9, 10, or 11 contiguous residues, up to the full sequence of the hinge region of trastuzumab, containing the sequence EPKSCDKTHTCPPCP (corresponding to residues 219-233 of SEQ ID NO:26).

1v) DOM1 h-131-206-GGGGSGGGGSGGGGS-Trastuzumab Fc

Provided herein is a TNFR1 antagonist fusion protein, containing the human TNFR1 antagonist dAb DOM1 h-131-206 (SEQ ID NO:59). The C-terminus of DOM1 h-131-206 is fused to a GGGGSGGGGSGGGGS peptide linker, which is fused to the N-terminus of the trastuzumab Fc region (corresponding to residues 234-450 of SEQ ID NO:26; see, also, SEQ ID NO:27). The DOM1 h-131-206-GGGGSGGGGSGGGGS-Trastuzumab Fc fusion protein has the following sequence (SEQ ID NO:723):

EVQLLESGGGLVQPGGSLRLSCAASGFTFAHETMVWVRQAPGKGL
EWVSHIPPDGQDPFYADSVKGRFTISRDNSKNTLYLQMNSLRAED
TAVYHCALLPKRGPWFDYWGQGTLVTVSSGGGGSGGGGSGGGGSA
PELLGGPSVFLFPPKPKDTLMISRTPEVTCVVVDVSHEDPEVKFN
WYVDGVEVHNAKTKPREEQYNSTYRVVSVLTVLHQDWLNGKEYKC
KVSNKALPAPIEKTISKAKGQPREPQVYTLPPSREEMTKNQVSLT
CLVKGFYPSDIAVEWESNGQPENNYKTTPPVLDSDGSFFLYSKLT
VDKSRWQQGNVFSCSVMHEALHNHYTQKSLSLSPGK

Alternatively, the GGGGSGGGGSGGGGS linker is replaced by a Gly-Ser linker, such as a (GSGS)ₙ or a (GGGGS)ₙ linker, where n=1, 2, 3, 4 or 5, or any other suitable short peptide linker as described herein or as known in the art.

1w) DOM1 h-131-206-GGGGSGGGGSGGGGS-SCDKTH-Trastuzumab Fc

Provided herein is a TNFR1 antagonist fusion protein, containing the human TNFR1 antagonist dAb DOM1 h-131-206 (SEQ ID NO:59). The C-terminus of DOM1 h-131-206 is fused to a GGGGSGGGGSGGGGS peptide linker, which is fused to a portion of the hinge sequence of trastuzumab, containing at least the sequence of residues SCDKTH (corresponding to residues 222-227 of SEQ ID NO:26), which is fused to the N-terminus of the trastuzumab Fc region (corresponding to residues 234-450 of SEQ ID NO:26; see, also, SEQ ID NO:27). The DOM1 h-131-206-GGGGSGGGGSGGGGS-SCDKTH-Trastuzumab Fc fusion protein has the following sequence (SEQ ID NO:724):

EVQLLESGGGLVQPGGSLRLSCAASGFTFAHETMVWVRQAPGKGL
EWVSHIPPDGQDPFYADSVKGRFTISRDNSKNTLYLQMNSLRAED
TAVYHCALLPKRGPWFDYWGQGTLVTVSSGGGGSGGGGSGGGGSS
CDKTHAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSREEMTK
NQVSLTCLVKGFYPSDIAVEWESNGQPENNYKTTPPVLDSDGSFF
LYSKLTVDKSRWQQGNVFSCSVMHEALHNHYTQKSLSLSPGK

Alternatively, the GGGGSGGGGSGGGGS linker is replaced by another Gly-Ser (GS) linker, such as a (GSGS)ₙ or a (GGGGS)ₙ linker, where n=1, 2, 3, 4 or 5, or any other suitable short peptide linker as described herein or as known in the art.

Alternatively, or additionally, the SCDKTH hinge sequence is replaced by all or a portion containing at least 5, 6, 7, 8, 9, 10, or 11 contiguous residues, up to the full sequence of the hinge region of trastuzumab, containing the sequence EPKSCDKTHTCPPCP (corresponding to residues 219-233 of SEQ ID NO:26).

1al) DOM1 h-131-206-ESKYGPPCPPCP-Nivolumab Fc

Provided herein is a TNFR1 antagonist fusion protein, containing the human TNFR1 antagonist dAb DOM1 h-131-206 (SEQ ID NO:59). The C-terminus of DOM1 h-131-206 is fused to the hinge sequence of nivolumab, containing the sequence ESKYGPPCPPCP (corresponding to residues 212-223 of SEQ ID NO:29), which is fused to the N-terminus of the nivolumab Fc region (corresponding to residues 224-440 of SEQ ID NO:29; see, also, SEQ ID NO:30). The DOM1 h-131-206-ESKYGPPCPPCP-Nivolumab Fc fusion protein has the following sequence (SEQ ID NO:738):

EVQLLESGGGLVQPGGSLRLSCAASGFTFAHETMVWVRQAPGKGL
EWVSHIPPDGQDPFYADSVKGRFTISRDNSKNTLYLQMNSLRAED
TAVYHCALLPKRGPWFDYWGQGTLVTVSSESKYGPPCPPCPAPEF
LGGPSVFLFPPKPKDTLMISRTPEVTCVVVDVSQEDPEVQFNWYV
DGVEVHNAKTKPREEQFNSTYRVVSVLTVLHQDWLNGKEYKCKVS
NKGLPSSIEKTISKAKGQPREPQVYTLPPSQEEMTKNQVSLTCLV
KGFYPSDIAVEWESNGQPENNYKTTPPVLDSDGSFFLYSRLTVDK
SRWQEGNVFSCSVMHEALHNHYTQKSLSLSLGK

Alternatively, all or a portion of the nivolumab hinge sequence, containing at least 5, 6, 7, 8, 9, 10, or 11 contiguous residues of the ESKYGPPCPPCP hinge sequence (corresponding to residues 212-223 of SEQ ID NO:29), is included.

### 1am) DOM1h-131-206-GGGGSGGGGSGGGGS-Nivolumab Fc

Provided herein is a TNFR1 antagonist fusion protein, containing the human TNFR1 antagonist dAb DOM1 h-131-206 (SEQ ID NO:59). The C-terminus of DOM1 h-131-206 is fused to a GGGGSGGGGSGGGGS peptide linker, which is fused to the N-terminus of the nivolumab Fc region (corresponding to residues 224-440 of SEQ ID NO:29; see, also, SEQ ID NO:30). The DOM1 h-131-206-GGGGSGGGGSGGGGS-Nivolumab Fc fusion protein has the following sequence (SEQ ID NO:739):

EVQLLESGGGLVQPGGSLRLSCAASGFTFAHETMVWVRQAPGKGL
EWVSHIPPDGQDPFYADSVKGRFTISRDNSKNTLYLQMNSLRAED
TAVYHCALLPKRGPWFDYWGQGTLVTVSSGGGGSGGGGSGGGGSA
PEFLGGPSVFLFPPKPKDTLMISRTPEVTCVVVDVSQEDPEVQFN
WYVDGVEVHNAKTKPREEQFNSTYRVVSVLTVLHQDWLNGKEYKC
KVSNKGLPSSIEKTISKAKGQPREPQVYTLPPSQEEMTKNQVSLT
CLVKGFYPSDIAVEWESNGQPENNYKTTPPVLDSDGSFFLYSRLT
VDKSRWQEGNVFSCSVMHEALHNHYTQKSLSLSLGK

Alternatively, the GGGGSGGGGSGGGGS linker is replaced by a (GSGS)ₙ or a (GGGGS)ₙ linker, where n=1, 2, 3, 4, or 5, or any other suitable short peptide linker as described herein or as known in the art.

1an) DOM1 h-131-206-GGGGSGGGGSGGGGS-ESKYGPPCPPCP-Nivolumab Fc

Provided herein is a TNFR1 antagonist fusion protein, containing the human TNFR1 antagonist dAb DOM1 h-131-206 (SEQ ID NO:59). The C-terminus of DOM1 h-131-206 is fused to a GGGGSGGGGSGGGGS peptide linker, which is fused to the hinge sequence of nivolumab, containing the sequence ESKYGPPCPPCP (corresponding to residues 212-223 of SEQ ID NO:29), which is fused to the N-terminus of the nivolumab Fc region (corresponding to residues 224-440 of SEQ ID NO:29; see, also, SEQ ID NO:30). The DOM1 h-131-206-GGGGSGGGGSGGGGS-ESKYGPPCPPCP-Nivolumab Fc fusion protein has the following sequence (SEQ ID NO:740):

EVQLLESGGGLVQPGGSLRLSCAASGFTFAHETMVWVRQAPGKGL
EWVSHIPPDGQDPFYADSVKGRFTISRDNSKNTLYLQMNSLRAED
TAVYHCALLPKRGPWFDYWGQGTLVTVSSGGGGSGGGGSGGGGSE
SKYGPPCPPCPAPEFLGGPSVFLFPPKPKDTLMISRTPEVTCVVV
DVSQEDPEVQFNWYVDGVEVHNAKTKPREEQFNSTYRVVSVLTVL
HQDWLNGKEYKCKVSNKGLPSSIEKTISKAKGQPREPQVYTLPPS
QEEMTKNQVSLTCLVKGFYPSDIAVEWESNGQPENNYKTTPPVLD
SDGSFFLYSRLTVDKSRWQEGNVFSCSVMHEALHNHYTQKSLSLS
LGK

Alternatively, the GGGGSGGGGSGGGGS linker is replaced by a (GSGS)ₙ or a (GGGGS)ₙ linker, where n=1, 2, 3, 4, or 5, or any other suitable short peptide linker as described herein or as known in the art. Alternatively, or additionally, all or a portion of the nivolumab hinge sequence, containing at least 5, 6, 7, 8, 9, 10, or 11 contiguous residues of the ESKYGPPCPPCP hinge sequence (corresponding to residues 212-223 of SEQ ID NO:29), is included.

Also synthesized were Vhh (nanobodies), particularly single chain forms thereof, linked to HSA, that comprises the dAbs, such as DOM1 h-131-206. Exemplary Vhh constructs were assessed for binding and inhibition of TNFR1. These are described in Examples 5-6 below.

### Example 4

Exemplary N-Terminal Fusion Protein of a Single Chain dAB with Human Serum Albumin and the Activities and Properties Thereof

Presentation in the context of an antibody presents difficulties in the context of inhibition of a target receptor. A bivalent antibody induces dimerization of a target receptor, which can lead to its activation. In some instances, as discussed in the detailed description, this is desirable, it is undesirable for an inhibitor of TNFR1. As described in the detailed description, even transient activation of TNFR1 can give rise to a cytokine storm and significant toxicity. Thus, in the context of this understanding a monovalent inhibitor should be used. To achieve this, monovalent constructs are provided herein (see description throughout, and Example 3 above). As described in the Example below, it is shown herein that there are bivalent constructs, such as the construct designated 1206-bivalent (see Example 5 below), which contains the Ab Dom1h-131-206 of SEQ ID NO:59, where n=2, linked via a linker to modified Fc's does not exhibit agonist activity, and, hence, does not induce dimerization of TNFR1. In contrast the corresponding construct 1208 does have agonist activity. See description below.

The cytokine network is defined by cascades of cytokines. As described throughout the disclosure herein, tumor necrosis factor a (TNF) is a key cytokine within the network of pro- and anti-inflammatory cytokines. Its role in promoting autoimmune disease is well-documented and discussed throughout the disclosure herein. TNF can trigger its own production as well as the release of interleukin 1 (IL-1), IL-6, IL-8, and other cytokines, which can induce other inflammatory factors. Endotoxins (lipopolysaccharide; LPS) from gram-negative bacteria are potent TNF-α inducers, and can induce sepsis. Viral infection can lead to an immune response that results in a cytokine storm of inflammatory mediators, triggered by TNF. Physical injury, including chemotherapy and surgery, can result in this outcome.

As described herein, these negative effects primarily are mediated through TNF/TNFR1 interactions. Opposing these effect are effects of TNF binding to its other receptor, TNFR2. TNFR1 is regarded as the proinflammatory TNF receptor; and TNFR2 is regarded as the anti-inflammatory TNF receptor. As discussed herein, TNFR2 has other pathophysiological functions. For instance, TNFR2 is critical for defense against opportunistic pathogens like tuberculosis and for maintenance of cardio myocyte function.

Commonly used anti-cytokine drugs are the TNF Blockers, such as, for example adalimumab (Humira®), etanercept (Enbrel®) and infliximab (Remicade®). These antibodies bind to TNF and prevent its binding to TNFR1 and to TNFR2. The severe toxicities associated with TNF Blockers are well-documented, and, as described herein, many of these toxicities result from inhibition of TNFR2 function. Constructs and products provided herein are designed to inhibit TNFR1 function, but not the function of TNFR2.

Constructs provided herein, including Vhh-4, exemplified below, and others are derived from Camelid Vhh domains. Vhh-4 is a Vhh anti-TNFR1 domain fused with the N-terminus of human serum albumin. The resulting construct provides the Vhh domain for blocking TNF, but, unlike prior dAbs, has sufficient half-life in vivo for its use as a therapeutic. Results below demonstrate that constructs, such as Vhh-4, are active in inhibiting TNF effects on cells that is at least as potent as the TNF Blockers adalimumab/Humira® and etanercept/Enbrel®.

This Example exemplifies the activities and properties of an exemplary construct, which is an N-terminal fusion protein of a single chain dAb with human serum albumin.

### A. Construct

This Example provides an exemplary nanobody and activities thereof. Nanobodies, as described in the detailed description, are Vhh domain-containing proteins, include only the heavy chain, and do not require cooperativity from a light chain, as is the case for humans and mice (Harmsen et al. (2007) Appl Microbiol Biotechnol. 77:13-22). Because they are a single chain, they must be presented as fusion proteins, such as grafted-CDRs in an antibody format, because of their short half-life on their own as small proteins (~13-15 KDa).

An exemplary construct was prepared. A phage library was prepared using the method described in Sabir et al. ((2014) Comptes Rendus Biologies 337:244-249). Phage with high affinity binding to the tumor necrosis factor receptor-1 (TNFR1) were recovered and tested for their ability to bind TNFR1, and to compete with binding of human tumor necrosis factor-alpha (TNF-α) for TNFR1 as described in U.S. Published Application No. US20140112929.

Nucleic acid encoding each of the Vhh antibodies, containing a single chain, was expressed in CHO cells (see Sokolowska-Wedzina et al. (2014) Protein Expression and Purification 99:50-57 for description of expression vector), and each was purified by HPLC chromatography. Sample 1 was a control anti-TNFR1 antibody (H398 from ThermoFisher), Vhh1-4 were Vhh antibodies that contain dAbs. The sequences (see SEQ ID NOs: 54, 1478, 58 and 59) are as follows:

Vhh-1:
EVQLLESGGGLVQPGGSLRLSCAASGFTFDKYSMGWVRQAPGKGL
EWVSQISDTADRTYYAHAVKGRFTISRDNSKNTLYLQMNSLRAED
TAVYYCAIYTGRWVPFEYWGQGTLVTVSS
Vhh-2:
EVQLLESGGGLVQPGGSLRLSCAASGFTFSQYRMHWVRQAPGKSL
EWVSSIDTRGSSTYYADPVKGRFTISRDNSKNTLYLQMNSLRAED
TAVYYCAKAVTMFSPFFDYWGQGTLV
Vhh-3:
EVQLLESGGGLVQPGGSLRLSCAASGFTFVDYEMHWVRQAPGKGL
EWVSSISESGTTTYYADSVKGRFTISRDNSKNTLYLQMNSLRAED
TAVYYCAKRRFSASTFDYWGQGTLVTVSS
Vhh-4:
EVQLLESGGGLVQPGGSLRLSCAASGFTFAHETMVWVRQAPGKGL
EWVSHIPPDGQDPFYADSVKGRFTISRDNSKNTLYLQMNSLRAED
TAVYHCALLPKRGPWFDYWGQGTLVTVSS

The highlighted and underlined Cysteines (C) in Vhh-4, and the corresponding cysteines in the other Vhh chains form a loop, whereby the Vhh is a constrained polypeptide. The proline at amino acid residue 14 can be replaced by alanine.

Expression cassettes encoding the Vhh domain antibody fragments were prepared and expressed, and purified by HPLC chromatography. The Table below shows the expression levels of each test Vhh antibody. Two molecules required a His tag for purification. Results are shown in the following Table:

TABLE 13
Expression and Yield Results for Vhh 1-4

| Item | Description | Tag | Harvest | Yield |
|---|---|---|---|---|
| Sample | Anti-human TNFRSF1A therapeutic | His | 146 μg | Abt. 1 |
| 1 | antibody scFv fragment (H398)* | | | mg/L |
| Vhh-1 | Recombinant human anti-TNFRSF1A | None | 500 μg | 30 |
| | single domain antibody | | | mg/L |
| Vhh-2 | Recombinant human anti-TNFRSF1A | His | 1000 μg | 49.4 |
| | single domain antibody | | | mg/L |
| Vhh-3 | Tandem scFV bispecific antibody | None | 500 μg | 42 |
| | | | | mg/L |
| Vhh-4 | Tandem scFV bispecific antibody | None | 500 μg | 51 |
| | (see SEQ ID NO: 1475) | | | mg/L |

*TNF receptor superfamily member 1A antibody H398 (ThermoFisher; H398 comprises SEQ ID NO: 678).

Each of Vhh 1-4 subsequently was tested in binding studies using the surface plasmon resonance (SPR) method (Sciences GL. Biacore Assay Handbook. General Electric Company (2012); and Richter et al. (2019) MAbs 11:166-177). Competition assays of Vhh inhibition of TNF-α binding to TNFR1 extracellular domain also were performed as described in Richter et al. (2019). Inhibition of TNF-induced expression of VCAM or IL8, which are comparable assays, were performed as described by Lin et al., (2015) J Biomed Sci 22: 53; and Sonnier et al. (2010) Journal of Gastrointestinal Surgery 14:1592-1599, respectively.

A summary of the results is presented in the Table below. The results show that Vhh-4 has a very high affinity for the extracellular domain of TNFR-1 (6.6×10⁻¹³ M); it was 100% competitive for TNF binding to TNFR1 (IC50~1 nM), and was the most efficient of the 4 candidates at inhibiting TNF-induced VCAM-1 synthesis (0.3 nM). Of these Vhh dAb antibodies tested, Vhh-4 performed the best and a construct containing the Vhh-4 and HSA was prepared.

The sequence below represents the construct (SEQ ID NO:1475) containing Vhh-4: the dAb portion is residues 20-138 of SEQ ID NO:1475, linked via a Gly-Ser linker (residues 139-147 of SEQ ID NO:1475) to human serum albumin (HSA; residues 148-732 of SEQ ID NO:1475).

EVQLLESGGGLVQPGGSLRLSCAASGFTFAHETMVWVRQAPGKGL
EWVSHIPPDGQDPFYADSVKGRFTISRDNSKNTLYLQMNSLRAED
TAVYHCALLPKRGPWFDYWGQGTLVTVSS . . . HSA. . . .

TABLE 14
Comparative binding and competition assays
(Results with the control antibody not shown*)

| | Kd | TNF competition | TNF Dependent |
|---|---|---|---|
| Sample | (Molar by SPR) | (maximum) | Bioassay (IC50) |
| Vhh-1 | 4.3 × 10⁻¹⁰ | Partial (33%) | IL-8 (500 nM) |
| Vhh-2 | 1.0 × 10⁻⁶ | Non-competitive | VCAM (3 nM) |
| Vhh-3 | 5.7 × 10⁻¹⁴ | Partial (34%) | IL-8 (1 nM) |
| Vhh-4 | 6.6 × 10⁻¹³ | Competitive (100%) | VCAM (0.3 nM) |

*amount of antibody recovered was de minimis

### B. Results

The exemplary construct containing the Vhh-4 antibody linked to HSA was shown to have activity as a TNFR1 antagonist. It blocked TNFR1 as shown by a significant reduction in IL-6 and IL-8 gene expression in THP1 cells stimulated with LPS. 3×10⁵ THP1 cells were treated with 5 μg and 20 μg, and 0 μg as a control, of the construct for 30 min, followed by LPS (10 ng/ml) stimulation for 4 hours. RNA samples were collected, and qPCR analysis was performed to analyze IL-6 and IL-8 gene expression using the housekeeping gene hypoxanthine-guanine phosphoribosyltransferase (HPRT) as an internal control. The results show that both doses significantly reduced IL-6 and IL-8 expression (n=3, mean±SEM; *p<0.05, **p<0.01, ***p<0.001).

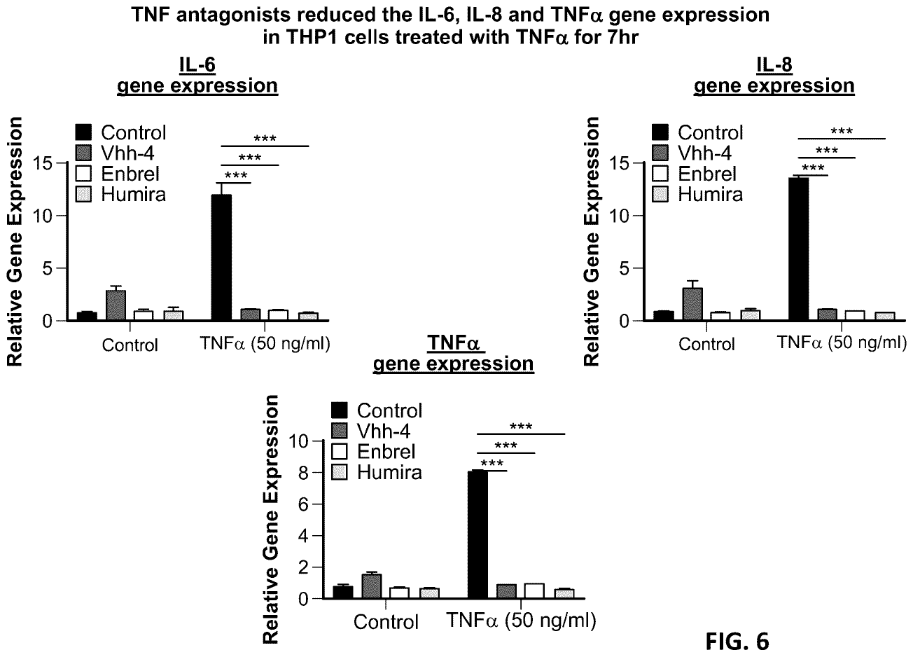

In other experiments, the effects on inflammatory cytokine expression of Vhh-4 on TNF-α stimulated THP-1 cells were compared to the effects of each of etanercept/Enbrel® and adalimumab/Humira©. 3×10⁵ THP1 cells were treated with 20 μg of Vhh-4, etanercept/Enbrel®, or adalimumab/Humira©, followed by TNF-α (50 ng/mL) simulation for 7 hours. RNA samples were performed and qPCR analysis was performed to IL-6, IL-8, and TNF-α gene expression, using the HPRT, a housekeeping gene product, as an internal control. Results, which are shown in FIG. 6 demonstrate that the Vhh-4 construct is at least as potent as etanercept/Enbrel® and adalimumab/Humira©. In other experiments, this construct had an IC₅₀ of about 9 nM.

### Example 5

Exemplary Vhh Domains and Fusion Proteins their Preparation and Properties Thereof

Various additional Vhh domains and fusion proteins were prepared to assess their properties. The results explain the clinical failure of prior art constructs, such as the dAbs (discussed and described in the Detailed Description) that contain a Vhh linked to anti-human serum albumin to bind the Vhh to human serum albumin.

### Materials

Synthesized Vhh domain proteins had the following sequences. The signal peptide in each construct is residues 1-19, linked for expression to a Vhh domain as follows:

Signal Peptide for secretion, MEWSWVFLFFLSVTTGVHS SEQ ID NO:1476, is a mouse immunoglobulin heavy-chain leader sequence (Uniprot: A0N1R). Other signal sequences can be used in its place as appropriate for expression in a cell.

### Constructs

206
(SEQ ID NO: 1493)
MEWSWVFLFFLSVTTGVHSEVQLLESGGGLVQPGGSLRLSCAASG
FTFAHETMVWVRQAPGKGLEWVSHIPPDGQDPFYADSVKGRFTIS
RDNSKNTLYLQMNSLRAEDTAVYHCALLPKRGPWFDYWGQGTLVT
VSSGGGGAGGGGHHHHHHHHHH
Residues 20-138 are amino acid residues 1-119
of SEQ ID NO: 59
541(208)
(SEQ ID NO: 1494)
MEWSWVFLFFLSVTTGVHSEVQLLESGGGLVQPGGSLRLSCAASG
FTFDKYSMGWVRQAPGKGLEWVSQISDTADRTYYAHAVKGRFTIS
RDNSKNTLYLQMNSLRAEDTAVYYCAIYTGRWVPFEYWGQGTLVT
VSSGGGGAGGGGHHHHHHHHHH
Residues 20-138 correspond to amino acid residues
1-119 of SEQ ID NO: 38
208a
(SEQ ID NO: 1495)
MEWSWVFLFFLSVTTGVHSEVQLLESGGGLVQPGGSLRLSCAASG
FTFDKYSMGWVRQAPGKGLEWVSQISDTADRTYYAHAVKGRFTIS
RDNSKNTLYLQMNSLRAGGGGAGGGGHHHHHHHHHH
Residues 20-107 correspond to amino acid residues
1-88 of SEQ ID NO: 38
019
(SEQ ID NO: 1496)
MEWSWVFLFFLSVTTGVHSQVQLQESGGGVVQPGGSLTLSCTRTG
LTPSTGAVGWYRQAPGKKCELVSYITIPSGRTTYTDSVKGRFAIS
RDKAKNTVFLQMNSLKPEDTALYYCGDVPYSTIQAMCTDDGPWGQ
GTQVTVSSGGGGAGGGGHHHHHHHHHH
Residues in italics are the same as SEQ ID NO: 40
in WO2021/256254 with one
change, a V, which is underlined.

### Methods

### Protein Expression

His-tagged proteins were expressed using a high expression mammalian vector transfected in suspension CHO cells and purified using Immobilized Metal Affinity Chromatography. Each completed construct was sequence-confirmed before proceeding to DNA scale-up. Suspension CHO cells (TunaCHO™) were seeded in a shake flask and expanded using a serum-free and chemically defined medium. On the day of transfection, the expanded cells were seeded into a new vessel with fresh medium. After transfection, the cells were maintained as a batch-fed culture until the end of the production run. There was no detectable 208a fragment in the medium of the cultured cells transfected with the corresponding construct. The Vhh domain in the 206 construct is the same as the domain in the Vhh-4-human serum albumin construct in Example 4.

### Plasmid DNA Scale-up

Each DNA expression construct was scaled up for transfection. Uncut plasmid DNA was analyzed by agarose gel electrophoresis and quality was assessed. Plasmid DNA was sequence-confirmed before proceeding to transfection.

### CHO Transient Transfection (TunaCHO™ Process)

Suspension CHO cells were seeded in a shake flask and expanded using a serum-free and chemically defined medium. On the day of transfection, the expanded cells were seeded into a new vessel with fresh medium. After transfection, the cells were maintained as a batch-fed culture until the end of the production run.

### IMAC (Immobilized Metal Affinity Chromatography) Purification of His Tagged PROTEIN

Clarified and buffered conditioned medium from the production run was loaded onto an IMAC column pre-equilibrated with binding buffer. Washing buffer containing 40 mM imidazole was passed through the column until the OD280 value returned to baseline. The target protein was eluted with a linear gradient of increasing imidazole concentration up to 0.5 M. The eluate was collected in fractions, and the OD280 value of each fraction was recorded. Denaturing capillary electrophoresis (CE-SDS, LabChip GXII, Perkin Elmer) of each fraction was performed and analyzed. Fractions containing the target protein were pooled and dialyzed into the client-specified buffer. The protein was filtered through a 0.2 μm membrane filter and the protein concentration was calculated using the OD280 value and the calculated extinction coefficient. Refer to the “Proteins Produced and Aliquots” section for a summary of the protein yield and corresponding information.

CE-SDS (Capillary Electrophoresis using Sodium Dodecyl Sulfate) Analysis

CE-SDS analysis of the target protein was performed using a LabChip GXII (Perkin Elmer). Refer to the Certificate of Analysis for results.

### SE-UPLC (Size Exclusion-Ultra High Pressure Liquid Chromatography) Analysis

SE-UPLC analysis of the target protein was performed. SEC standards (MEDNA, Y3101) were chromatographed as a reference for protein sizes. Refer to the Sample Analysis Report for more details and results.

### Protein Purification

Clarified and buffered conditioned medium from each production run was loaded onto an IMAC column pre-equilibrated with binding buffer. Washing buffer containing 40 mM imidazole was passed through the column until the OD280 value returned to baseline. The target protein was eluted with a linear gradient of increasing imidazole concentration up to 0.5 M. The eluate was collected in fractions, and the OD280 value of each fraction was recorded. Denaturing capillary electrophoresis (CE-SDS, LabChip GXII, Perkin Elmer) of each fraction was performed and analyzed. Fractions containing the target protein were pooled and dialyzed into the client-specified buffer (PBS (137 mM NaCl, 2.7 mM KCl, 10 mM Na2HPO4, 2 mM KH2PO4, pH 7.4). The protein was filtered through a 0.2 m membrane filter and the protein concentration was calculated using the OD280 value and the calculated extinction coefficient.

The pI of each of the proteins was calculated:
206: theoretical pI of 6.69
541 (208): theoretical pI of 7.22
208a
019: theoretical pI of 7.79
The Vhh-4-HSA fusion protein (SEQ ID NO:1475) has a theoretical pI of 5.75.

This is of interest because at the pI (isoelectric point) the negative and positive charges in a protein are balanced reducing repulsive electrostatic forces, and the attraction forces predominate, causing aggregation and precipitation (see, e.g., Proteomic Profiling and Analytical Chemistry (Second Edition), 2016). Proteins that have a pI close to the pH of blood (pH 7.4) will aggregate; hence the Vhh domains, which have a pH close to that of blood, will aggregate upon administration. The fusion protein with HSA has much lower pI, and will not aggregate.

### Results

### Vhh Proteins

Post IMAC purification the proteins were buffer exchanged to 20 mM Histidine 150 mM NaCl pH 6.0 and yield obtained were ~19 mg of 206, ~4 mg of 541(208) and ~23 mg of 019; however, no target protein was captured and enriched for208a. No detectable level of 208a in the CM was observed based on CE-SDS analysis.

Proteins were buffer exchanged to PBS pH 7.4. Post buffer exchange the final yield obtained were 10.76 mg of 206, 1.04 mg of 541(208), and 20.67 mg of 019. SE-UPLC analysis was performed and 019 was observed to contain >99% monomers. SE-UPLC results 206 and 541(208) were inconclusive, which might have been caused by column interactions. SE-UPLC analysis was performed and all proteins. The results showed a very significant loss of proteins post buffer exchange for 206 due to aggregation.

### Vhh-4 Human Serum Albumin Construct

The Vhh-4 construct in Example 4, contains the same Vhh domain as 206 above, which has a pI close to that of blood, and which aggregated. The DNA encoding the construct described in Example 4 (Vhh-4 linked to human serum albumin) was cloned into the high expression mammalian vector and the DNA sequences of the gene insert were confirmed. Each DNA construct was scaled up for transfection and the DNA sequence was confirmed after DNA scale-up. A 0.1 liter transient production was completed in CHO cells (TunaCHO™ extended 14-day process). The protein was purified by Anti-Albumin purification and 26.75 mg (1.07 mg/mL) the Vhh-4 fusion protein were obtained. CE-SDS analysis was performed. SE-UPLC analysis was performed and all proteins were observed to contain >97% monomers. No aggregates were observed indicating that fusion Vhh-4-HSA maintains the unaggregated form of Vhh-4.

These results provide insights into the clinical failure of the prior art constructs that contained the Vhh domains linked to an anti-human serum albumin. As occurred in buffer at pH 7.4, for 206 and the other domains, as described above, exposure to serum, which has a pH of about 7.4, in vivo can induce aggregation, resulting in rapid clearance and very little or no association of a Vhh-4 protein with the targeted free human serum albumin. Constructs provided herein in which the unaggregated form of the Vhh-4 protein is maintained, solve this problem.

### Example 6

Engineering and Production of EN1206 Variants Protein engineering

EN1206-MV and EN1206-BV are VHH-Fc fusion proteins that were engineered for in vivo half-life extension. To do so, a triple mutation of YTE (M252Y/S254T/T256E) was engineered in the Fc portion of the fusion protein. A second triple mutation was also made in the Fc for the purpose of silencing the antibody effector function. These mutations are known as “LALAPG,” which refers to the re[placements, by EU numbering: L234A/L235A/P329G. A review article for YTE and LALAPG is (frontiersin.org/articles/10.3389/fimmu.2019.01296/full). The Fc portions of EN1206-MV has the Knob-into-Hole mutations to facilitate its heterodimer pairing (see FIG. 8A for a depiction of the structures of EN1206-MV and -BV).

To eliminate crosslinking of FcγRs on T cells to alleviate the cytokine induction from T cells the Fc region of antibodies has been modified to eliminate FcγR binding (see, e.g., Saunders (2019) Front. Immunol., Sec. Comparative Immunology 10 doi.org/10.3389/fimmu.2019.012) A single mutation of Leu235Glu is sufficient to knockout binding to Fc receptors. A 100-fold reduction in binding to FcγR also results in lower T cell activation and proliferation in the presence of the Leu235Glu Fc mutant IgG1. The combination of Leu234Ala and Leu235Ala (called LALA mutations) eliminates FcγRIIa binding (see Table below). These two mutations eliminate detectable binding to FcγRI, IIa, and IIIa for IgG1 and for IgG4. LALA is more effective than either Leu234Ala or Leu235Ala alone. Some groups have seen that the Leu235Glu mutation knocks out FcγRI. Single and double mutations at position 234 and 235 reduced ADCC activity mediated by PBMCs and nearly ablated ADCC mediated by monocytes, the LALA mutations have been tested in humans in a phase I clinical trial. A pairing, in addition to the LALA pairing, is Ser228Pro paired with Leu235Glu, which has been called the SPLE or PE mutations. The SPLE mutations have been introduced into IgG4, which has low binding to FcγR initially perhaps due to the Phe234 residue that differs from Leu234 found in IgG1. (This combination has been shown by surface plasmon resonance to dramatically reduce FcγRI binding to IgG Fc to barely detectable levels without reducing circulating half-life in rats. The effect of the Ser228Pro improves stability of IgG4. Table 3 reproduced from Saunders (2019) Front. Immunol., Sec. Comparative Immunology Volume 10, doi.org/10.3389/fimmu.2019.01296:

| | | | Reduced |
|---|---|---|---|
| | Abbreviated | | effector |
| Modifications or mutations | name | Phenotype | function |
| Leu235Glu | LE | Decreases binding to cell surface | ADCC |
| | | FcyRs | |
| Leu234Ala/Leu235Ala | LALA | Decreases binding to FcyRI, II, III | ADCC |
| | | | ADCP |
| | | | CDC |
| Ser228Pro/Leu235Glu | SPLE in IgG4 | Decrease FcyRI binding | |
| | | Half-life unchanged | |
| Leu234Ala/Leu235Ala/Pro329Gly | LALA-PG | Eliminates binding to FcyR1, II, | ADCP |
| | | Ill, C1q | |
| Pro331Ser/Leu234Glu/Leu235Phe | TM | Decreases binding to FcyR1, II, III | CDC |
| | | and | |
| | | C1q | |
| Asp265Ala | DA | Decreases binding to FcyR1, II, III | ADCC |
| | | | ADCP |
| Gly237Ala | | Decreases binding to FcyRII | ADCP |
| Glu318Ala | | Decreases binding to FcyRll | ADCP |
| Glu233Pro | | Decreases binding to FcyRI, II, | |
| | | and III | |
| Gly236Arg/Leu328Arg | GRLR | Decreases binding to all FcyR | ADCC |
| IgG2-IgG4 cross-subclass | IgG2/G4 | Decreases binding to FcyRs and | |
| | | C1q | |
| His268Gln/Val309Leu/Ala330Ser/Pro3 | IgG2m4 | Decreases binding to all FcyR | ADCC |
| 31Ser | | | |
| | | Decreases Cig binding | ADCP |
| | | | CDC |
| Va1234A1a/Gly237Ala/Pro238Ser/ | IgG2a | Near complete elimination of | ADCC |
| His268AlaNa1309Leu/Ala330Ser/Pro331Ser | | FcyRI, Ila, Ilb, and Ills binding | ADCP |
| | | Decreases C1q binding | CDC |
| | | Binds FcRn | |
| Leu234Ala/1235Ala/Gly237Ala/P238 | IgGla | Near complete elimination of | ADCC |
| Ser/His268Ala/Ala330Ser/Pro331Ser | | FcyR1, Ila, Ilb, and Ills binding | CDC |

A Pro329Gly mutation has been added to the LALA mutations; this mutation inhibits binding to murine FcγRI, II, and III by IgG2a Fe. The amino acid at 329 was changed, because this residue makes contact with Trp108 and Trp131 of FcγRIIIa. The LALA-PG is an improvement over LALA mutations alone because they nullify Fc function in mouse and human IgG; LALA alone retains murine FcγRIII binding to murine IgG2a. As a result, the LALA-PG mutations are that observed results in murine models more accurately translate to humans since the mutations confer a similar phenotype for both murine IgG2a and human IgG1.

### Production of Variants Provided Herein

The constructs designated EN1206-MV (KiH-YTE-LALAPG) and EN1206-By (YTE-LALAPG) were produced through transient expression in CHO cells at 1 liter scale. Their expression levels, purity, and total yields were excellent. The proteins are currently stored at WuXi for future delivery. A summary table is shown below to demonstrate the protein sizes and their purities.

| | | | | Final | |
|---|---|---|---|---|---|
| | | | | Concentration | mL/ |
| | Total yield/L | Purity | Endotoxin | (mg/mL) | vial |
| EN1206-MV | 1445.76 mg | 99.6% | <0.11 EU | 10.4 | 4 |
| EN1206-BV | 2228.08 mg | 98.2% | <0.07 EU | 7.96 | 4 |

Four VHH and VHH-Fc fusion proteins were tested to determine the extent of TNFR1 and TNFR2 binding (See exemplary schematic constructs in FIGS. 8A and 8B). Sequences are set forth below. The leader sequences, which are underlined, are included for trafficking upon expression, and are cleaved, and not included in the constructs when isolated.
1) EN-1000-206 (‘206’—a VhH single domain polypeptide); see FIG. 8A, top left panel.
2) EN-1206-Fc-MV (a knobs in hole construct pairing an IgG1 Fc-only domain to a fusion of 206-IgG1-Fc); see FIG. 8B, left-most panel.
3) EN-1206-Fc-BV (homodimeric/bivalent 206-IgG1-Fc); see FIG. 8B, center panel.
4) EN-1208-Fc-BV: Same as EN-1206-Fc-BV except the VhH domain was EN-1000-208 (containing the dAb of SEQ ID NO:54), a different VhH domain fused with IgG1-Fc to form a bivalent antibody-like protein. 1208 is binds to a different epitope on TNFR1 than EN-1206 constructs. See FIG. 8B, right most panel.

### Summary of Results and Methods

Experiment Section 1: Engineering, Production and Target Binding of the Constructs

Three VHH/Fc fusion proteins were engineered and produced; about 30 mg each was prepared. The constructs are the one-armed EN-1206-Fc-MV (also referred to as 206 VHH-KiH Fc or EN1206-MV), EN-1206-Fc-BV (206VHH Fc or EN1206-BV), and EN-1208-Fc-BV (also referred to as 208 VHH-Fc or EN1208-BV). Schematic constructs are depicted in FIGS. 8A and 8B. In the table below the constructs are collectively referred to as EN1000.

Three of the EN1000 proteins were manufactured by CreativeBio. The manufacturing specifications, purity and formulation are presented in the Table below:

TABLE EN1000
Protein Yield and Purity

| | Yield (mg) | mg/vial | Total vial | Purity | Endotoxin | Formulation |
|---|---|---|---|---|---|---|
| EN1206-OA | 30 | 1 | 30 | 94 | <1 EU/mg | PBS, pH 7.4 |
| EN1206 | 30 | 1 | 30 | 94 | <1 EU/mg | PBS, pH 7.5 |
| EN1208 | 30 | 1 | 30 | 99 | <1 EU/mg | PBS, pH 7.6 |

SEQ ID NO: 1488
>EN1206 monovalent seq1_-hole
Fc only, Hole (T366S, L368A, Y407V), YTE (M252Y, S254T, T256E),
LALAPG (L234A, L235A, P329G)
MGWSCIILFLVATATGVHSDKTHTCPPCPAPEAAGGPSVFLFPPKPKDTLYITREPEVT
CVVVDVSHEDPEVKFNWYVDGVEVHNAKTKPREEQYNSTYRVVSVLTVLHQDWL
NGKEYKCKVSNKALGAPIEKTISKAKGQPREPQVYTLPPSRDELTKNQVSLSCAVKG
FYPSDIAVEWESNGQPENNYKTTPPVLDSDGSFFLVSKLTVDKSRWQQGNVFSCSVM
HEALHNHYTQKSLSLSPGK
SEQ ID NO: 1489
>EN1206 monovalent seq2_-knob
One-armed VHH-Fc, Knob (T366W), YTE (M252Y, S254T, T256E),
LALAPG
(L234A, L235A, P329G)
MGWSCIILFLVATATGVHSEVQLLESGGGLVQPGGSLRLSCAASGFTFAHETMVWV
RQAPGKGLEWVSHIPPDGQDPFYADSVKGRFTISRDNSKNTLYLQMNSLRAEDTAVY
HCALLPKRGPWFDYWGQGTLVTVSSDKTHTCPPCPAPEAAGGPSVFLFPPKPKDTL
YITREPEVTCVVVDVSHEDPEVKFNWYVDGVEVHNAKTKPREEQYNSTYRVVSVLT
VLHQDWLNGKEYKCKVSNKALGAPIEKTISKAKGQPREPQVYTLPPSRDELTKNQVS
LWCLVKGFYPSDIA VEWESNGQPENNYKTTPPVLDSDGSFFLYSKLTVDKSRWQQG
NVFSCSVMHEALHNHYTQKSLSLSPGK
SEQ ID NO: 1490
>EN1206 bivalent
YTE (M252Y, S254T, T256E) LALAPG (L234A, L235A, P329G)
MGWSCIILFLVATATGVHSEVQLLESGGGLVQPGGSLRLSCAASGFTFAHETMVW
VRQAPGKGLEWVSHIPPDGQDPFYADSVKGRFTISRDNSKNTLYLQMNSLRAEDTAV
YHCALLPKRGPWFDYWGQGTLVTVSSDKTHTCPPCPAPEAAGGPSVFLFPPKPKDT
LYITREPEVTCVVVDVSHEDPEVKFNWYVDGVEVHNAKTKPREEQYNSTYRVVSVL
TVLHQDWLNGKEYKCKVSNKALGAPIEKTISKAKGQPREPQVYTLPPSRDELTKNQV
SLTCLVKGFYPSDIAVEWESNGQPENNYKTTPPVLDSDGSFFLYSKLTVDKSRWQQG
NVFSCSVMHEALHNHYTQKSLSLSPGK
SEQ ID NO: 1491
>EN1208 YTE-LALAPG
YTE (M252Y, S254T, T256E) LALAPG (L234A, L235A, P329G)
MGWSCIILFLVATATGVHSEVQLLESGGGLVQPGGSLRLSCAASGFTFDKYSMGWV
RQAPGKGLEWVSQISDTADRTYYAHAVKGRFTISRDNSKNTLYLQMNSLRAEDTAV
YYCAIYTGRWVPFEYWGQGTLVTVSSDKTHTCPPCPAPEAAGGPSVFLFPPKPKDTL
YITREPEVTCVVVDVSHEDPEVKFNWYVDGVEVHNAKTKPREEQYNSTYRVVSVLT
VLHQDWLNGKEYKCKVSNKALGAPIEKTISKAKGQPREPQVYTLPPSRDELTKNQVS
LTCLVKGFYPSDIAVEWESNGQPENNYKTTPPVLDSDGSFFLYSKLTVDKSRWQQGN
VFSCSVMHEALHNHYTQKSLSLSPGK
SEQ ID NO 54:
Polypeptide sequence of Dom 1h-574-208
SEQ ID NO: 54 (also SEQ NO: 651 and 667)
EVQLLESGGGLVQPGGSLRLSCAASGFTFDKYSMGWVRQAPGKGLEWVSQISDTAD
RTYYAHAVKGRFTISRDNSKNTLYLQMNSLRAEDTAVYYCAIYTGRWVPFEYWGQ
GTLVTVSS
SEQ ID NO: 1492 Polypeptide sequence of DOM1h-131-206
(also SEQ ID NO: 59)
EVQLLESGGGLVQPGGSLRLSCAASGFTFAHETMVWVRQAPGKGLEWVSHI
PPDGQDPFYADSVKGRFTISRDNSKNTLYLQMNSLRAEDTAVYHCALLPKRG
PWFDYWGQGTLVTVSS

Knob-into-hole mutations are engineered into the monovalent EN1206-MV for its heterodimer paring. YTE mutations are engineered into the Fe to extend its in vivo half-life of the constructs. The LALAPG mutations also are engineered into the Fes to minimize its in vivo Fe effector function.

Target Binding with Protein A Sensor Chip

The following shows that these three VHH/Fc proteins above bind only the TNF receptor type 1 (TNFR1) but not TNF receptor type 2 (TNFR2), and that the binding to TNFR1 blocks the binding of the TNFR1 ligands (TNF-α and lymphotoxin (TNF-β)) to TNFR1.

### SPR Binding Format

Series S Sensor Chip Protein A (Cytiva #29127556) was used to immobilize the Fc portion of the VHH/Fc proteins. The human soluble TNFR1 and TNFR2 are used for the SPR binding flow.

### Results:

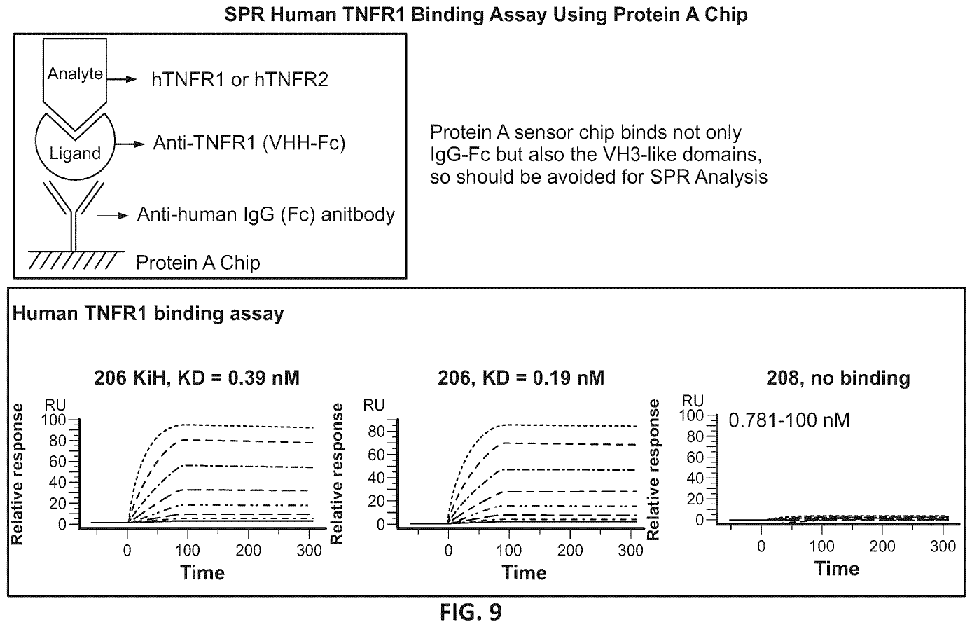

EN1206-MV and EN1206-BV bind TNFR1 with high affinity, while EN1208 shows no binding to TNFR1 (FIG. 9 and Table A below). The EN1208 results were repeated with a non-protein A Chip to rule out a known interaction of protein A with VH3-like domain (in which the VhH antigen-binding regions are embedded). There is no binding of the three EN1000 proteins to TNFR2 (data not shown).

TABLE A
SPR Human TNFR1 Binding Assay Results Using Protein A Chip

| Immobilized | Analyte | | | | |
|---|---|---|---|---|---|
| VHH-Fc | (Concentration) | Ka | Kd | KD(M) | Fit Model |
| EN1206-MV | TNFR1 | 4.27E+05 | 1.66E−04 | 3.88E−10 | 1:1 binding |
| | (0.781-100 nM) | | | | |
| EN1206-BV | TNFR1 | 3.24E+05 | 5.99E−05 | 1.85E−10 | 1:1 binding |
| | (0.781-100 nM) | | | | |
| EN1208-BV | TNFR1 | n/a | n/a | No Binding | — |

### ELISA Competitive Binding

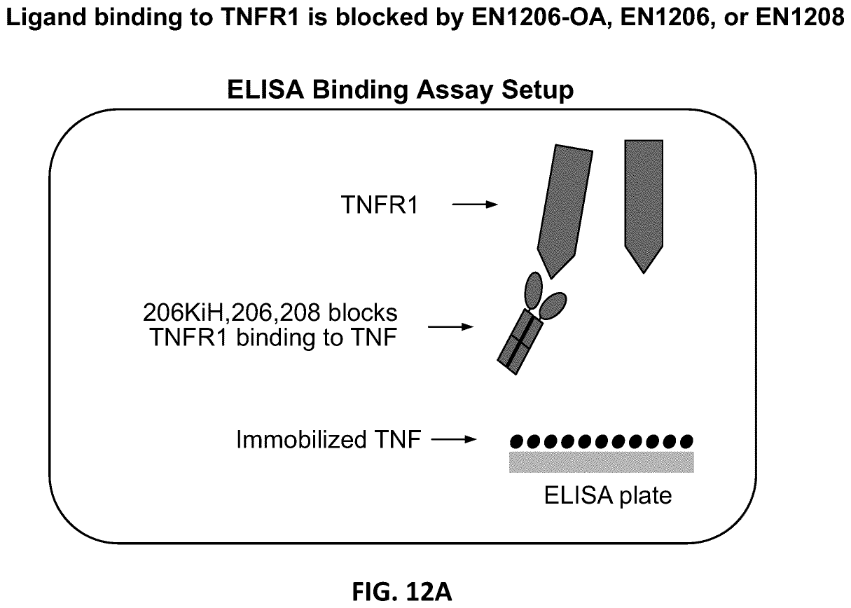

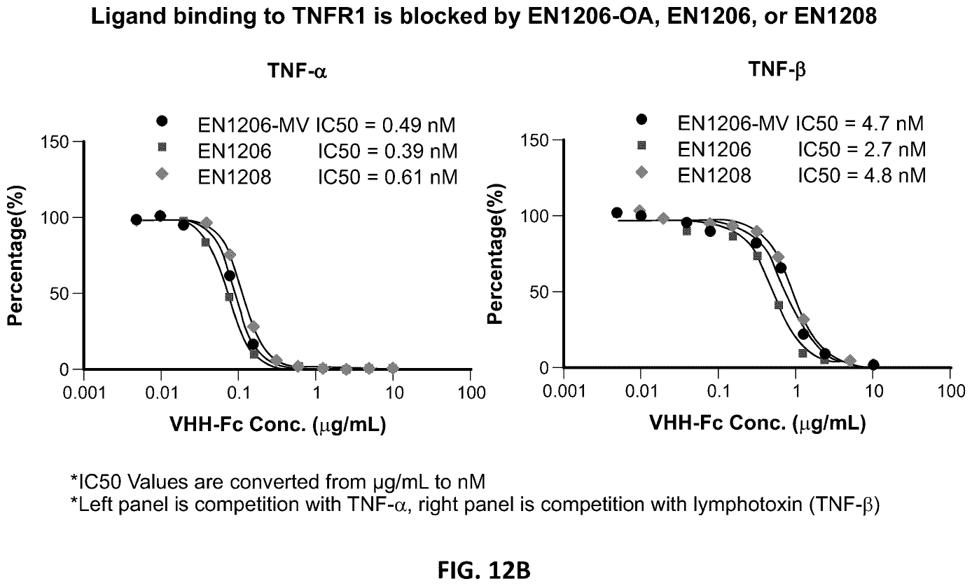

The TNFR1 ligands TNF-α and lymphotoxin (TNF-β) are immobilized on the ELISA plate. The TNFR1 binding was performed in the presence of increasing amounts of the VHH/Fc proteins. It is demonstrated that the binding of TNFR1 to the immobilized TNF-α or lymphotoxin (TNF-β) can be blocked by either EN1206-MV (also referred to EN1206-OA for “one-armed), or EN1206, or EN1208 in a dose-dependent manner (FIG. 12B). This result further suggests that there is an artifact resulting in the apparent non-binding of EN-1208-Fc-BV to TNFR1 as otherwise it is difficult to understand how it can compete with TNF binding to TNFR1.

Target Binding using a CM5 Sensor Chip

### Background

SPR binding starts with choosing a matching sensor chip. Previous SPR binding of EN1206 and EN1208 used a protein A sensor chip. The known interaction between protein-A and V_{H}3-like domains (cytivalifesciences.com/en/us/solutions/bioprocessing/knowledge-center/vh3-and-fc-fragment-binding-to-protein-a). Therefore, the binding assay was repeated with a non-protein A sensor chip, CM5.

### Materials and Methods

CM5 sensor chip (Cytiva #BR100530) was coated with an anti-human IgG (Fc) antibody (Cytiva #29234600) to capture the EN1206-BV and EN1208-BV. The human soluble TNFR1 was used as analyte for binding flow.

### Results and Discussion

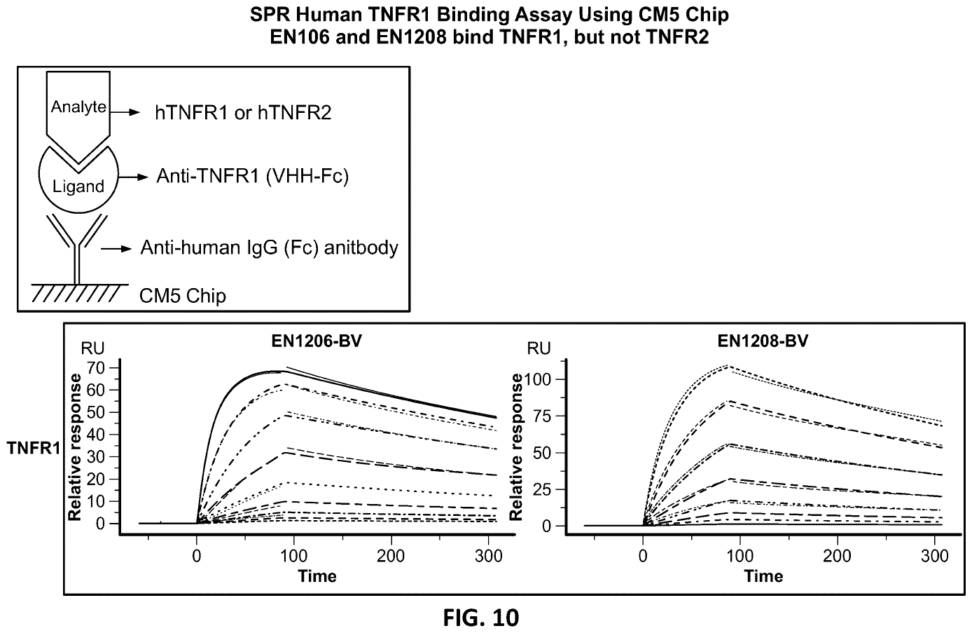

The SPR binding of EN1206 and EN1208 to the soluble TNFR1 using the CM5 sensor chip is shown in FIG. 10 and Table B below. Exemplary data in FIG. 10 and Table B below show that the constructs EN-1206-BV and EN-1208-BV bind TNFR1 but not TNFR2. EN-1206-BV demonstrates higher binding affinity for hTNFR-1 than EN-1208-BV. EN1208-BV has a slower on-rate resulting in a ~100× poorer affinity (KD) than that of EN1206-BV.

TABLE B
SPR Human TNFR1 Binding Assay Results Using CM5 Chip

| Immobilized | | | | |
|---|---|---|---|---|
| VHH-Fc | Analyte | Ka | Kd | KD(M) |
| EN1206-BV | TNFR1 | 1.15E+06 | 1.71E−03 | 1.49E−09 |
| EN1208-BV | TNFR1 | 9.46E+03 | 2.12E−03 | 2.24E−07 |
| EN1206-BV | TNFR2 | n/a | n/a | No Binding |
| EN1208-BV | TNFR2 | n/a | n/a | No Binding |

Experiment Section 2: EN1206 is a potent TNFR1 antagonist Background

Anti-TNF-α treatment such as Adalimumab blocks both TNFR1 (inflammatory) and TNFR2 (anti-inflammatory) resulting in serious side effects. TNFR1-specific antagonistic constructs provided herein benefit broader patient populations.

### Materials and Methods

THP1 monocytic leukemia cell line is the test cell line and is used in experiments that measure the expression of inflammatory cytokines in vitro under laboratory conditions. Anti-TNFR1 agents used are 1) EN206 (206): a humanized single domain VHH (nanobody) that binds to domain 4 of TNFR1; 2) EN1206: a bivalent 206/Fc fusion protein; 3) EN1208: a bivalent 208/Fc fusion protein, wherein 208 is a humanized nanobody binding to domain 3 of TNFR1; and 4) EN1206-MV: a one-armed (monovalent) 206/Fc bearing the Knob-into-Hole mutations. TNF-α was purchased. The treated cells were subject to RNA extraction followed by qPCR analysis of the TNF-α-induced gene expression of CXCL8 (IL-8) and TNF-α.

The 5×10⁵ THP1 cells were treated with indicated dose of anti-TNFR1 agents for 30 min, followed by TNF-α (100 ng/ml) stimulation for 6 hr. The RNA samples were collected, and qPCR analysis was performed to analyze genes expression using HPRT as internal control (n=3, mean±SEM). The treated cells were subject to RNA extraction followed by qPCR analysis of the TNF-α-induced gene expression of CXCL8 (IL-8) and TNF-α.

### Results and Discussion

### THP1 Assay

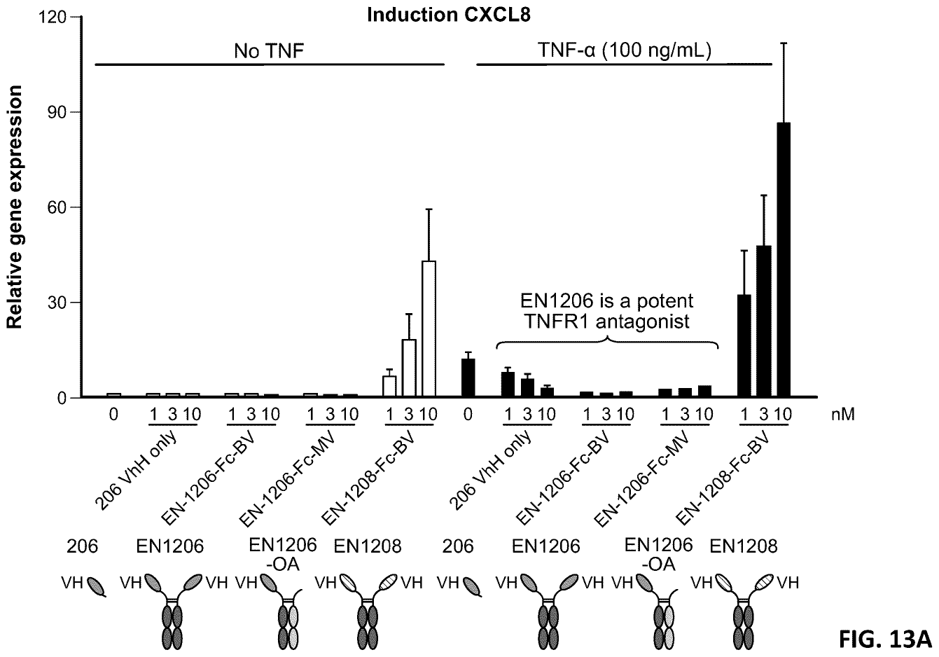

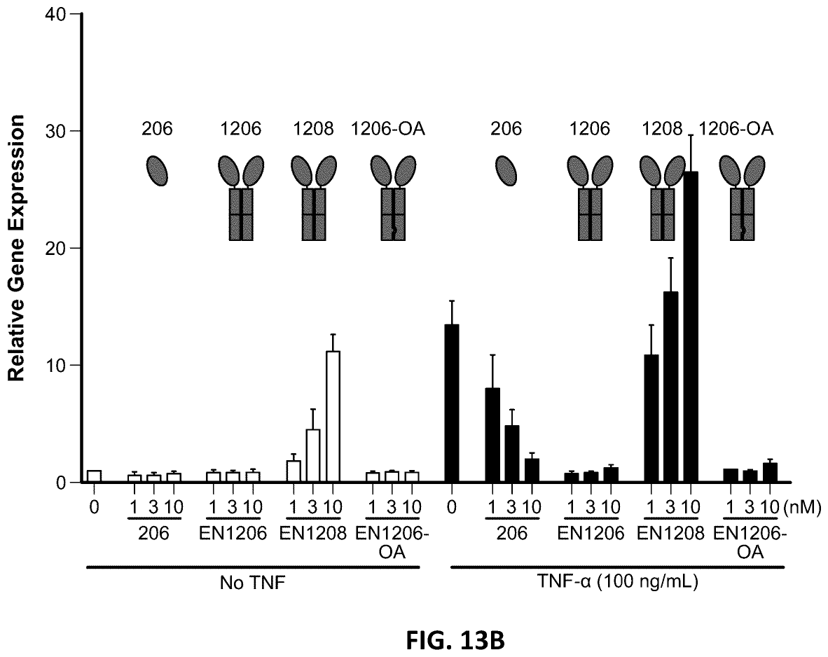

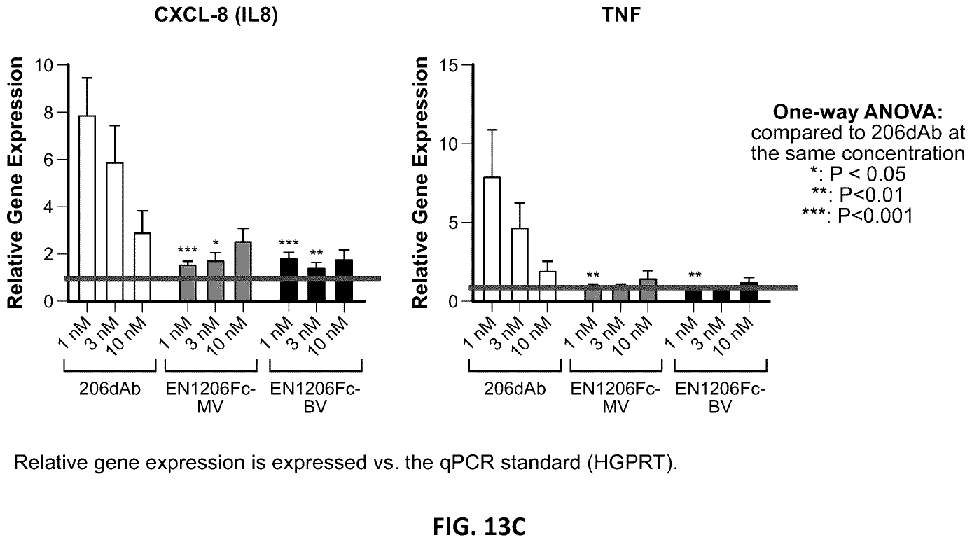

The THP1 cell line, an in vitro cell model for immune modulation study, was treated with the anti-TNFR1 agents including the 206, EN1206-Fc-BV (IgG1 Fc fusion in bivalent format; EN1208-Fc-BV and EN1206-Fc-MV. After 30 min, the cells were further treated with or without TNF-α for 6 hours. Expression of the TNF-a-inducible genes CXCL8(IL8) and TNF-α were analyzed by qPCR using HGPRT as internal control (FIG. 13C).

The results (FIGS. 13A-13C) showed that EN1206 and EN1206-OA are more potent than the 206 (the dAb) in inhibiting CXCL8 and TNF-α gene expression following TNF-α stimulation. The results also show that EN1208 alone induces CXCL8 and TNF-α gene expression. The results show that EN1206, such as EN-1206-FC-BV and EN-1206-FC-MV, are potent TNFR1 antagonists. Such antagonists can be used as next generation treatments for autoimmune diseases, disorders, and/or conditions in which TNF-α plays a role. In contrast, the results show that EN-1208 is an agonist of the TNF-α mediated gene expression. A difference between the EN1206s and the EN1208s is the epitope with which each interacts (see, Example below).

FIG. 13C shows data plotted as relative gene expression of either IL8/CXCL8 or TNF of 206 dAb, EN1206-Fc-MV, and EN1206-Fc-BV, as measured by qPCR. THP1 cells (5×10⁵) were treated with test reagents at concentrations of 1 nM, 3 nM, and 10 nM for 30 minutes, followed by TNF (100 ng/ml) stimulation for 6H. RNA was extracted and qPCR analysis was performed to analyze gene expression of IL-8 or TNF using HPRT as an internal control (n=3, mean+/−SEM). For I1L8, 1 nM 206dAB is 8-fold the baseline (Value=1 horizontal line). The 1 nM value for EN-1206-Fc-MV is 1.75, and EN-1206-Fc-BV is 1.75. The background subtracted values are shown in the Table inserted into FIG. 13C. CXCL8(IL8) was inhibited by EN-1206-Fc-MV and EN-1206-Fc-BV, while EN-1000-206-VhH (EN206) showed inhibition of gene expression to a lesser extent. EN-1208 acted as an agonist by inducing both CXCL8 and TNF.

All three molecules, EN-1000-206 dAb, EN-1206-Fc-MV and EN-1206-Fc-BV, suppress TNF-induced IL8 and TNF gene expression in THP1 monocytic leukemia cells in vitro in a dose dependent manner. The 206 dAb has been employed characterized previously in a double-domain antibody (ddAb) with the structure anti-TNFR1 dAb-(spacer)-anti-HSA dAb (Jakopin et al., Int. J. Mol. Sci. 2019, 20, 4265). The affinity of the anti-HSA portion is reported as 34 nM. The purpose of ddAb binding to HSA is to obtain an HSA-like half-life for the fusion protein. Cordy et al., Clinical and Experimental Immunology, 182: 139-148 (2015), report that the half-life in patients was 20-30 hours, which is likely due to the low affinity (34 nM) of the ddAb for HSA, and other factors.

This problem is addressed herein by fusing the anti-TNFR1 206 dAb to the Fc domain of IgG1. This was also done to avoid the reactivity of the variable heavy chain (dAb)C-terminus of 206 dAb with pre-existing anti-Vh (variable heavy chain) protein sequences (Rossotti et al. The FEBS Journal 289 (2022) 4304-4327). This resulted in the higher potency of EN-1206-Fc-MV and EN-1206-Fc-BV.

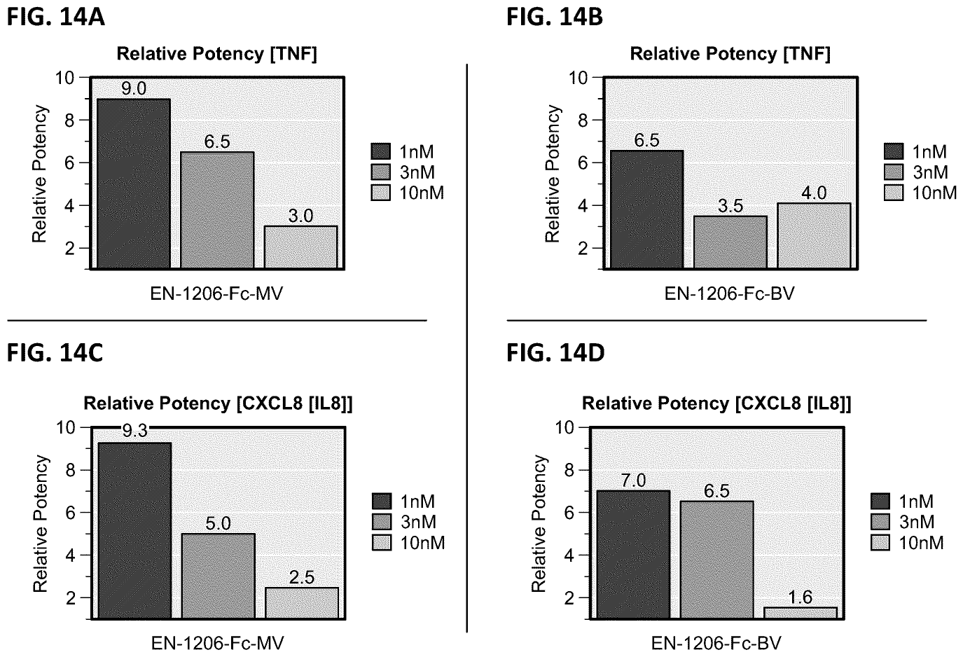

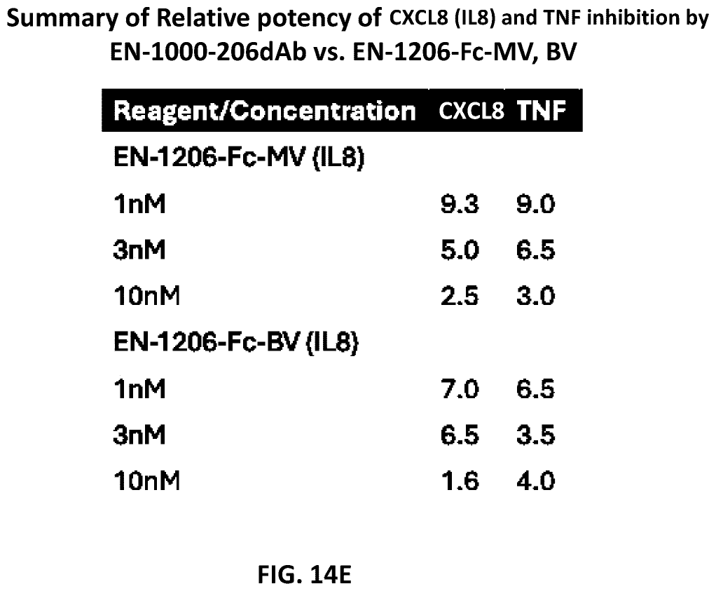

FIG. 14E summarizes the data shown in FIG. 14 (A, B, C, and D). For suppression of TNF-induced expression of IL8/CXCL8, EN-1206-MV (1 nM) is 9.3 fold more potent than 206dAb, while 1 nM EN-1206-Fc-BV is 7-fold more potent. Similar results are shown for TNF-induced expression of TNF. EN-1206-Fc-MV (1 nM) is 9-fold more potent than dAb206, and 1 nM EN-1206-Fc-BV is 6.5-fold more potent than 206dAb.

Improved potency occurs at all concentrations of the EN-1206-Fc molecules, although the differences are reduced with higher concentrations of inhibitor. This result could not have been predicted from previous work with these molecules, such as described by Enever et al (Enever et al. Protein Engineering, Design & Selection, 2015, vol. 28 no. 3, pp. 59-66) and Cordy et al (Cordy et al, Clinical and Experimental Immunology, 182: 139-148 (2015). Because higher potency occurs both in the monovalent and bivalent forms of EN-1206-Fc it cannot be the result of multivalency but the because of or resulting from stabilization of a more potent conformation of the variable heavy chain nanobody.

### Additional Results

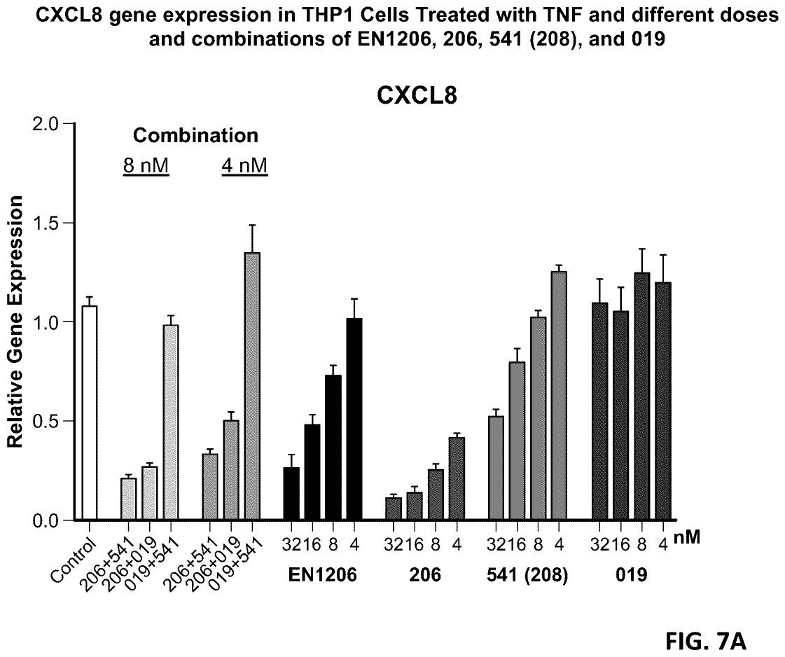

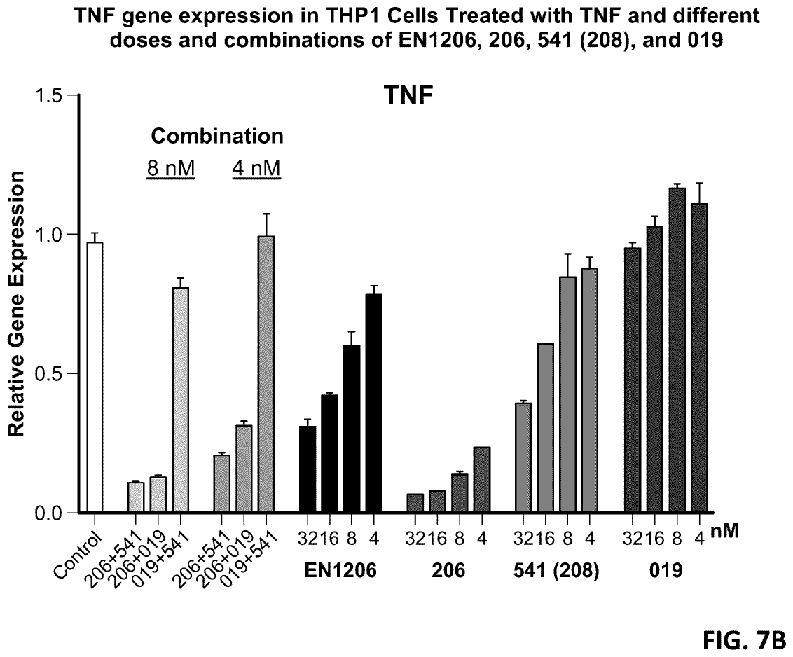

Exemplary data in FIGS. 7A and 7B demonstrate that three (3) out of five (5) VhH/VhH-serum albumin candidate molecules suppress the TNF-induced inflammatory cytokine release in vitro. Anti-TNFR1 constructs and combinations of constructs were evaluated for their ability to suppress induction of expression of downstream inflammatory cytokines, CXCL8(IL8) and TNF. The constructs tested included: 1) EN1206, an anti-TNFR1 VhH (206) fusion protein with human serum albumin; 2) EN206 (206), a humanized single domain VHH (nanobody) that binds to domain 4 of TNFR1; 3) 541 (designated elsewhere as 208), a humanized nanobody binding to domain 3 of TNFR1; and 4) 019 a dAB that binds TNFR1.

3×10⁵ THP1 cells were treated with En-1206, 206, 541, 019 alone (at 32, 16, 8, and 4 nM concentrations), or with combination treatments: 206+541, 206+019, and 541+019 (at 8 ant 4 nM concentrations), for 30 minutes. After anti-TNFR1 treatment cell were given an TNF-α (50 ng/mL) stimulation for 7 hr. The RNA samples were collected and qPCR analysis was performed to analyze the IL-8 ant TNF-α gene expression using HPRT as an internal control (n=3, mean+SEM).

The results show that the 206 peptide (EN206) is more potent than the fusion protein, indicating that the HSA portion of the fusion protein can interfere with folding of the VhH domain. The 541(208) peptide showed lower activity than the 206 VhH, indicating that it appears to bind to another epitope on TNFR1. The 019 peptide showed little activity and was not investigated further. Because 208(541) and 206 containing constructs bind to different epitopes on TNFR1, different combinations were tested to identify any synergistic effects. No synergy was observed.

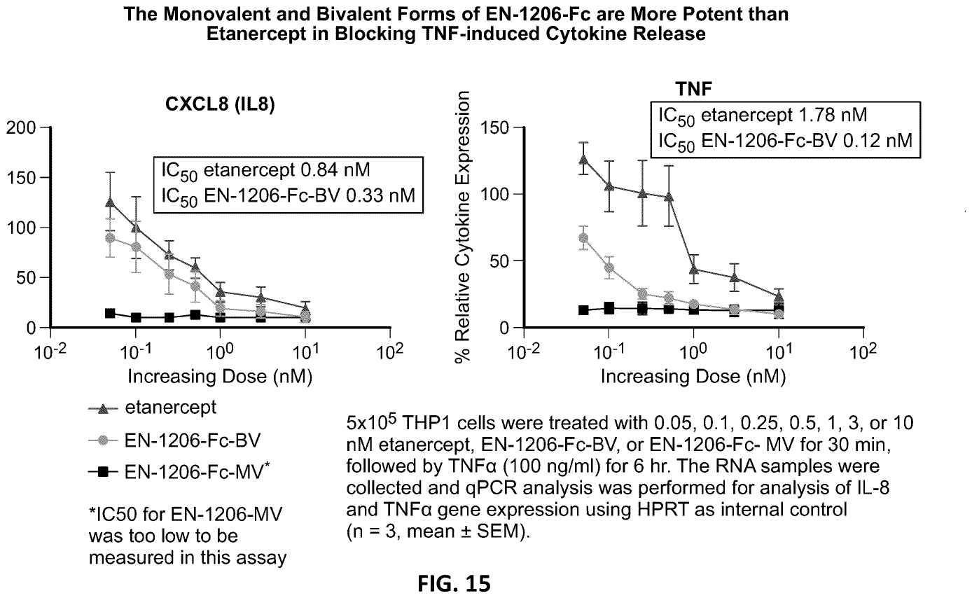

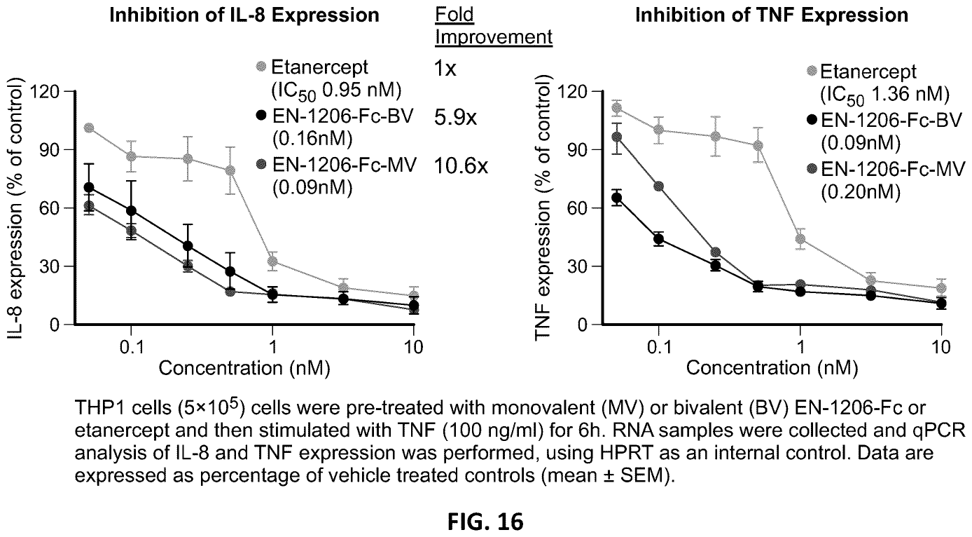

The 206 and 208 peptides were examined in additional experiments (see Examples above and below) and in other constructs (discussed and provided herein) that contain, as detailed herein, the dAbs linked to Fc's modified for increased half-life and to minimize in vivo Fc effector functions. Exemplary data in FIGS. 15 and 16 demonstrate a dose-dependent inhibition of TNF-α-induced gene expression in the THP1 cells.

In the first experiment, the ability of monovalent and bivalent constructs of EN-1206-Fc to inhibit TNF-induced cytokine release was compared to Etanercept (Enbrel®), a drug marketed for treating autoinflammatory conditions such as rheumatoid arthritis. 5×10⁵ THP1 cells were treated with 0.05, 0.1, 0.25, 0.5, 1, 3, or nanomolar concentration (nM) of Etanercept, EN-1206-Fc-Bv, or EN-1206-Fc-MV for 30 minutes followed by TNF-α (100 ng/mL) for 6 hours. The RNA samples were collected and qPCR analysis was performed to detect expression levels of IL-8 and TNF-α. HPRT was used as an internal control. FIG. 15 demonstrates the monovalent and bivalent forms of EN-1206-Fc are more potent than etanercept in blocking TNF-induced cytokine release.

A second experiment was performed to confirm the first, see FIG. 16. THP1 cells (5×10⁵) cells were pre-treated with monovalent (MV) or bivalent (BV) EN-1206-Fc or Etanercept and then stimulated with TNF (100 ng/ml) for 6h. RNA samples were collected, and qPCR analysis of IL-8 and TNF expression was performed, using HPRT as an internal control. Data are expressed as percentage of vehicle treated controls (mean±SEM). The results show that EN206-MV and EN1206-BV are more potent in inhibiting IL-8 and TNF expression than the positive control Etanercept (sold under the trademark Enbrel®). EN-1206-Fc-BV demonstrated 5.9-fold and 15-fold improvements in inhibition of IL-8 and TNF expression, respectively. En-1206-Fc-MV demonstrated a 10.6× and 6.8× fold improvement in inhibition of IL-8 and TNF expression, respectively.

An advantage of the EN-1206-Fc constructs provided herein is that, Etanercept and other TNF Blockers bind membrane-associated TNF and induce outside to inside signaling, resulting in expression of inflammatory cytokines; this does not occur with the EN-1206-Fc molecules.

### Example 6

### Comparative Data and Additional Description of Differences from the Prior Art

In these experiments, molecules tested were: 206 (anti-TNFR1 VHH domain only, EN-1000-206, the HSA construct (the 206 dAb linked to human serum albumin), 206 fused with the modified Fc (EN-1206-Fc-MV, monovalent), EN-1206-Fc-BV (bivalent), and EN-1208-BV. Previous work leads those of skill in the field of autoimmune disease to expect that bivalency in anti-TNFR1 antibody constructs leads undesirable to activation of TNFR1 (agonist activity) because of receptor clustering by the antibody construct. It is known that some antibodies are partially agonistic, while others are fully agonistic (see, e.g., Richter et al., PMID 23977237, Richter et al., PMID 30252601, Belka et al., PMID 7720706, Mandik-Nayak et al., doi.org/10.4049/jimmunol.167.4.1920, Yu et al., doi.org/10.1038/s42003-021-02309-5).

As discussed above, U.S. publication 2014/0112929A1 (Batuwangala et al.) descries and details the agonistic activity of bivalent TNFR1 binding antibody constructs, states that such antibodies are not useful as inhibitors of TNFR1. The published application states: “[a]ccordingly, multivalent agents that bind TNFR1 are generally not effective antagonists of TNFR1 even if they block the binding of TNF-α to TNFR1.” The publication describes monovalent inhibitors of TNFR1, which are derived from a VhH library. Because the conformation of the VhH domain is dependent upon its context of presentation (VhH peptide alone; VhH affinity linked to HSA; VhH fused with human serum albumin (HSA); VhH fused with IgG Fc in either a monovalent or bivalent configuration), it is described and shown herein that the same VhH domain has different interactions with its TNFR1 target.

As described and shown herein various presentation modalities (VhH alone, VhH fused with HSA, VhH fused with IgG1 Fc in a monovalent and bivalent format) have been explored. Agonism vs. antagonism can be epitope dependent. Some mAbs bind to TNFR1 causing a conformational change in the receptor such that it cannot activate downstream pathways, even in the presence of TNF or lymphotoxin (LT or TNF-P). This mechanism has been reported for the trastuzumab-HER2 receptor tyrosine kinase complex, which mechanism leads to inhibition of signal transduction because of conformational changes that prevent receptors from activating the receptor-associated tyrosine kinase. The TNF receptors belong to the cytokine receptor family and do not have an inherent tyrosine kinase. They, thus, are regulated differently from HER2. As a result, cytokine receptors do not suffer suppression of signaling with monoclonal antibody binding. US application publication 2014/0112929 states that:
agents that bind TNFR1, such as anti-TNFR1 antibodies, have additionally been shown to induce TNFR1 clustering and signal transduction in the absence of TNF and are commonly used as TNFR1 agonists. (See, e.g., Belka et al., EMBO, 14(6): 1156-1165(1995); Mandik-Nayak et al., J. Immunol, 167:1920-1928(2001). Accordingly, multivalent agents that bind TNFR1 generally are not effective antagonists of TNFR1 even if they block the binding of TNF to TNFR1.

Thus, according to the prior art anti-TNFR1 constructs are not considered as candidate therapeutic agents for treatment of any of the myriad of diseases, disorders, or conditions in which TNF plays a role in the etiology or in which TNFR1 is a target. In contrast, however, it is shown herein, that contrary to this dogma, TNFR1 constructs that do not have agonist activity can be prepared. The EN1206 constructs, including the 1206 bivalent construct exemplified herein are TNFR1 antagonists, and do not have TNFR1 agonist activity.

Having shown herein that it is possible to prepare TNFR1 antagonist constructs that do not have TNFR1 agonist activity, other constructs that contain TNFR1 binding moieties, such as dAbs can be prepared by identifying dAbs that bind to the same epitope as that to which the dAb in the 1206 constructs (SEQ ID NO:59) binds, and/or by preparing constructs and, using the assays and methods herein, identifying those that do not have agonist activity. As detailed below, numerous dAbs that bind to the same epitope as the 1206 constructs are identified; constructs containing those dAbs, particularly constructs containing the dAbs linked to the modified Fc's in bivalent and monovalent forms as detailed herein,

Using the SPR binding format with the extracellular domains of TNFR1 and TNFR2, dose-dependent and high affinity and specificity for TNFR1 was demonstrated; the latter by showing there was no binding to TNFR2. EN-1206-Fc-MV and EN-1206-Fc-BV also were shown to block the binding of TNFR1 ligands (TNF-α and TNF-β/lymphotoxin). EN1206-Fc MV and BV were shown to inhibit TNF-α-induced gene expression in human THP1 cells. These results indicate that EN-1206-Fc-MV and EN-1206-Fc-BV are potent TNFR1 antagonists. In contrast, EN-1208-Fc, on the other hand, despite its ability to block TNF binding to TNFR1, was agonistic and induced TNF-α-mediated activation.

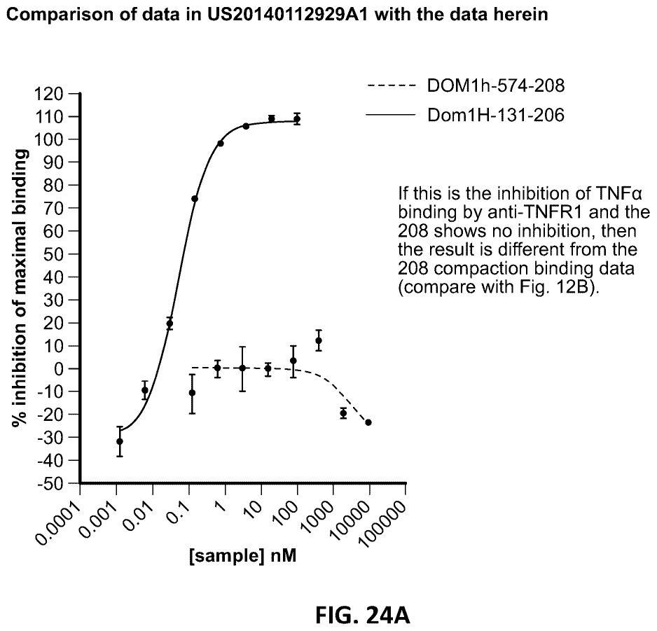

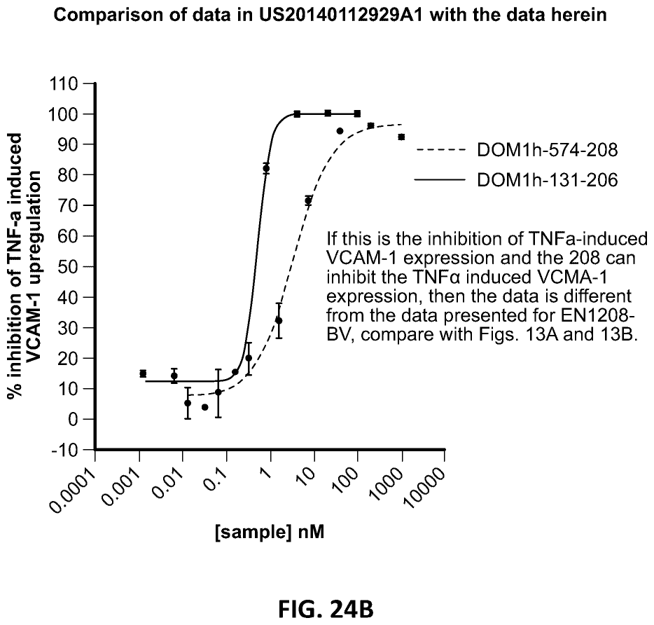

US publication 2014/0112929 (Batuwangala et al.), discussed above, reports that DOM1H-574-208 (SEQ ID NO:667, containing the 208 dAb) does not compete with TNF-α for binding to TNFR1 (FIG. 24A). This is in contrast to results herein that show that that EN1208-Fc-BV construct competes with TNF-α and with lymphotoxin (TNF-0) for binding to TNFR1 (see, FIG. 12B compare with FIG. 24A). In a TNF-α functional assay, Batuwangala et al. show that the DOM1H-574-208 is capable of inhibiting TNF-α signal transduction and inhibited the TNF-α-induced VCAM-1 upregulation (see, FIG. 24B). These results show that DOM1H-574-208 acts as a TNFR1 antagonist. This should be compared with the results herein that show that in the context of the constructs herein, EN208-Fc-BV BV acts as an agonist (see, FIGS. 13A and 13B and compare with FIG. 24B).

Batuwangala describes that one immunoglobulin single variable domain, identified in International PCT publication No. WO2011051217 as DOM1 h-574-208 (SEQ ID NO:2 therein and SEQ ID NO:54 herein), was identified as an example of a non-competitive TNFR1-specific binding protein. An example of a competitive TNFR1 binding protein is the heavy chain (Vh) dAb DOM1 h-131-206 (SEQ ID NO:54 herein), identified in WO2008149148.

Thus, provided herein are constructs that are TNFR1 antagonists, that are not TNFR1 agonists, and also those that do not antagonize TNFR2. They can be prepared from known components, including dAbs and other VhH antibodies, and linked directly or optionally via a linker, to Fc's, modified for improved properties, as described herein, to produce bivalent constructs that do not agonize TNFR1. Other dAbs are identified herein that can be included in constructs in place of the 206 dAb.

### Example 7

### Determination of TNFR1 Epitope Binding for EN1206 and EN1208

### SUMMARY

An important part of antibody drug discovery and development efforts is finding the target binding specificity and epitope diversity.EN1206 and EN1208 are VHH-Fc fusion proteins that target TNF Receptor Type 1 (TNFR1). The epitope binning results detailed herein demonstrated that EN1206 and EN1208 bind to separate, non-overlapping epitopes on TNFR1. Such differential binding can contribute to the different properties of the EN1206 and EN1208 constructs.

### Materials

A summary of candidate molecules tested and the Human TNFR1 protein used for binding is shown in the table below. Notably, recombinant TNFR1 used for these experiments was expressed in HEK293 cells to ensure binding is most representative of binding to native human protein.

| | Manufactured | Commercial |
|---|---|---|
| Reference | for Applicant | catalog |
| EN1206-mv | 70269_1W230508 | \ |
| EN1208-bv | 70329_1W230519 | \ |
| Human TNFR1/CD120a | \ | SinoBiologicals: |
| protein, His & Avi tag | | 10872-H27H-B |

### Methods

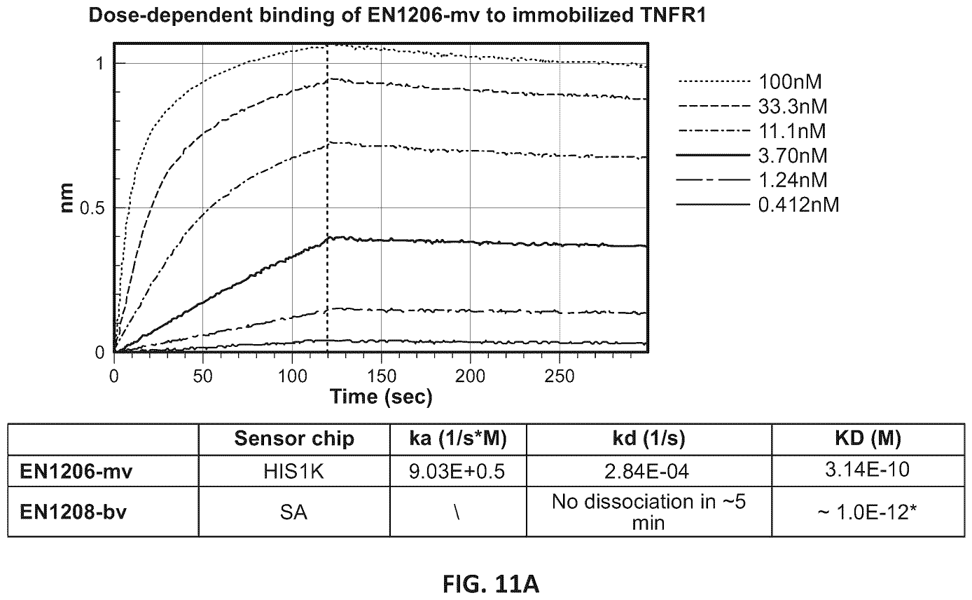

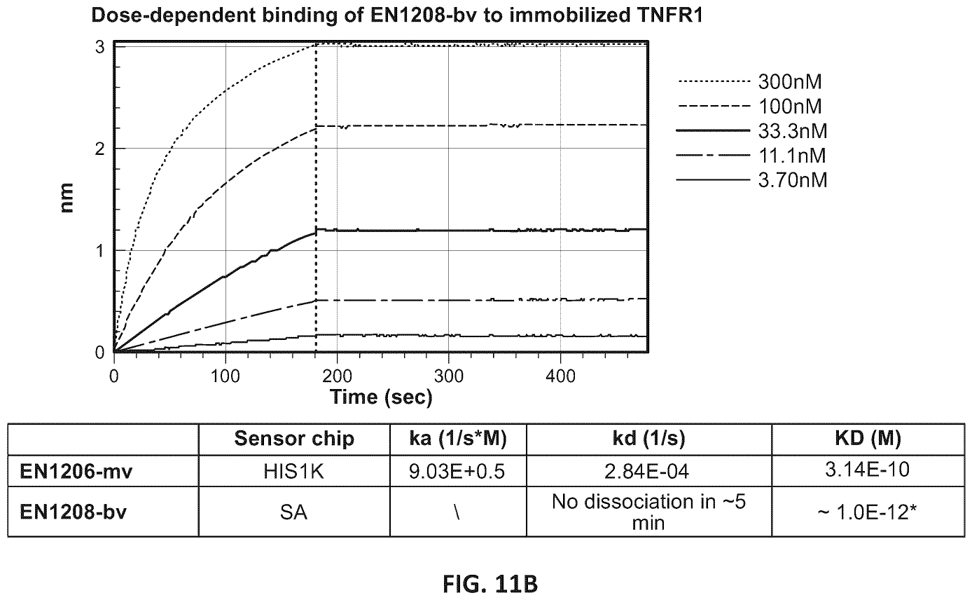

An Octet instrument RED96 was used for the assay. Octat anti-penta His biosensor (HIS1K) and Octet Streptavidin (SA) biosensor were used to immobilize the TNFR1-His-avi tag protein. An SA sensor was used for EN1208 binding since EN1208 showed non-specific binding to the HIS1K sensor. Octet RED96 was used to measure the binding affinity of EN1206-mv and EN1208-bv. A dose-dependent binding of EN1206-mv to the immobilized TNFR1 on HIS1K chip was detected within the concentration range of 0.412 nM to 100 nM (FIG. 11A). A dose-dependent binding of EN1208-bv to the immobilized TNFR1 on SA chip was detected within the concentration range of 3.7 nm to 300 nM (FIG. 11B).

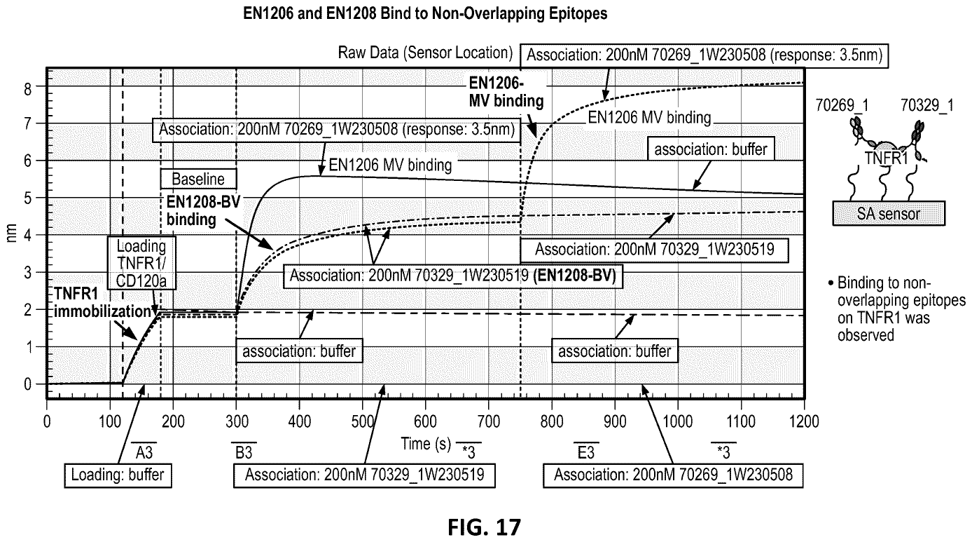

Epitope binning immunoassays using surface plasmon resonance (SPR) was used to determine if EN1206-mv and EN1208-bv bind to the same epitope or non-overlapping epitopes. For the epitope binning assay shown in FIG. 17, the Streptavidin (SA) biosensor chip was used to immobilize the TNFR1. This was followed by binding of the EN1208-bv (the 1st antibody) and EN1206-mv (the 2nd antibody), sequentially, to immobilized TNFR1 on the sensor surface. As can be seen, EN1208-BV and EN1206-MV generated non-overlapping sensor response curves demonstrating binding to non-overlapping epitopes.

### Results and Discussion

The experiments started with the determination of the binding specificity and affinity of EN1206 and EN1208 to the immobilized human soluble TNFR1. A concentration-dependent binding of EN1206 and EN1208 to the TNFR1 was observed, respectively (Table 15, FIGS. 11A-11B). Due to a lack of detectable dissociation of EN1208-BV after its binding to the immobilized TNFR1 within the dissociation assay time frame (~5 min), the on-rate (ka) and off-rate (kd) could not be calculated. Thus, an affinity (K_{D}) for EN1208 was estimated (Table 15).

TABLE 15
The binding affinity of EN1206 and EN1208 to the immobilized
soluble human TNFR1

| | Sensor chip | ka (1/s*M) | kd (1/s) | KD (M) |
|---|---|---|---|---|
| EN1206-mv | HIS1K | 9.03E+0.5 | 2.84E−04 | 3.14E−10 |
| EN1208-bv | SA | \ | No dissociation in | ~1.0E−12* |
| | | | ~5 min | |

*No dissociation was detected in the assay time frame, so the KD for EN1208 was estimated.

The results demonstrate that EN1206/Fc-mv and EN1208/Fc-BV bind to the non-overlapping, separate epitopes. Early-stage Ab drug development efforts often generate many leads.

### Example 8

### Suppression of TNF-Induced Cytokine Expression by EN-1206 SUMMARY

The results herein show that EN-1206 suppress cytokine release almost to baseline by preserving TNFR2 activity. Specific down regulation of TNFR1 by EN-1206-Fc-MV gives a more complete response rate.

### Materials and Methods

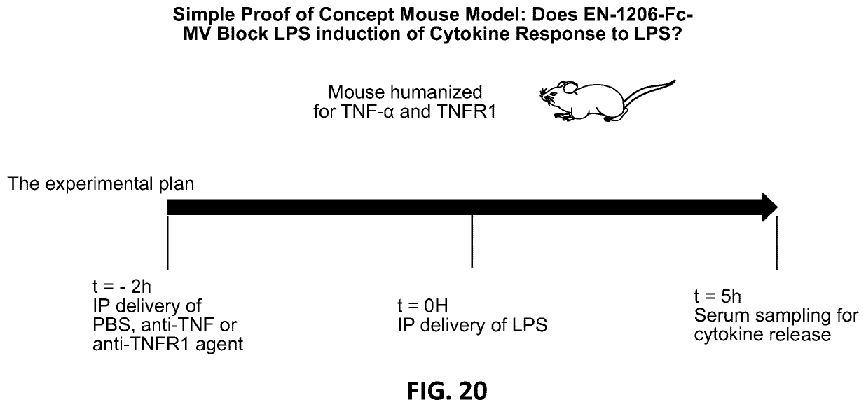

Studies are performed with humanized (HuTNFR1/HuTNF) transgenic mice, n=4-8 per treatment group. Mice were treated with the following: vehicle (PBS, phosphate buffer saline), 10 mg/kg of anti-TNF agent (the antibody sold under the trademark Inflectra® antibody, a generic of anti-TNF antibody Infliximab sold as Remicade®), or 30 mg/kg or 3 mg/kg anti-TNFR1 agent (EN-1206-Fc-MV). Vehicle and treatments were administered via intraperitoneal injection (i.p.) 2 hours prior to administration of 10 μg lipopolysaccharide (LPS) to stimulate TNF-Induced cytokine release. Serum was collected 5 hours following LPS stimulation. See FIG. 20.

### Results and Discussion

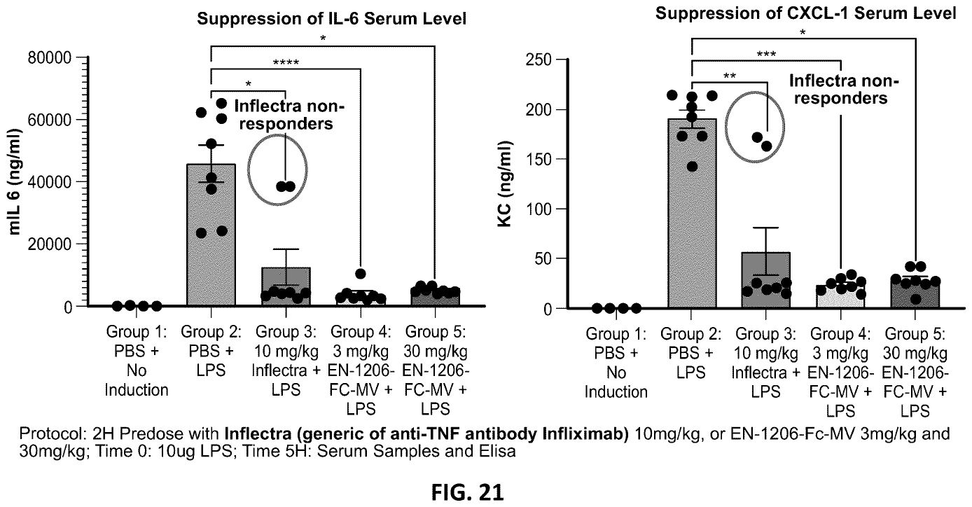

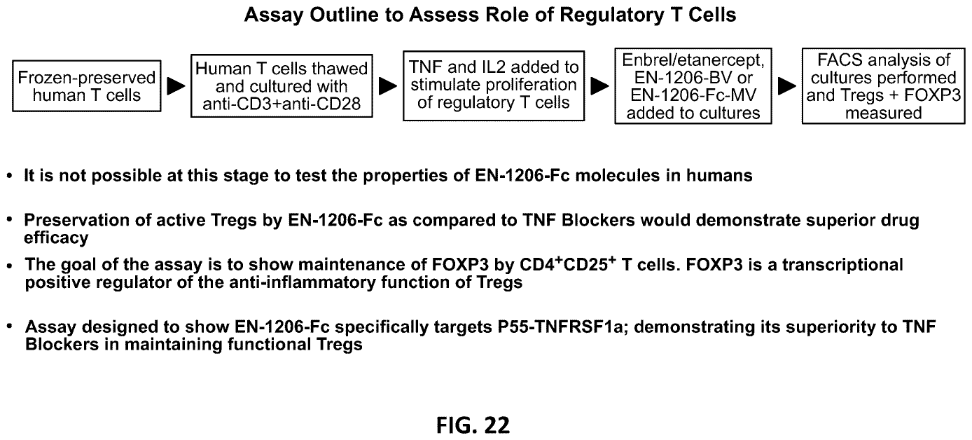

The results demonstrate that EN-1206-Fc-MV at high and low doses inhibits TNF-Induced cytokine release in vivo, see FIG. 21. EN-1206-Fc-MV treatment effected a complete response, whereas 2/8 mice were non-responsive the Infliximab (Inflectra®) treatment groups. Inflectra® antibody is a biosimilar of Infliximab/Remicade® antibody, which is known to leave behind about 20% non-responders. The incomplete response observed in previous anti-TNF agents is due to the co-repression of TNFR2. Commonly used anti-TNF agents, such as, for example adalimumab (sold under the trademark Humira®), etanercept (sold under the trademark Enbrel®) and infliximab (trademark Remicade®) bind to TNF and prevent its binding to TNFR1 and TNFR2. The severe toxicities associated with TNF Blockers are well-documented, and, as described herein, many of these toxicities result from inhibition of TNFR2 function. As demonstrated herein, constructs provided herein do no inhibit TNFR2 function.

### Example 9

Effect of EN-1206BV-Fc, EN-1206MV-Fc, and Entanercept (Enbrel® antibody) on maintaining Treg populations induced by the TNF-α cytokine.

### Summary

Known anti-TNF agents, such as, for example adalimumab (Humira®), etanercept (Enbrel®) and infliximab (Remicade®), bind to TNF and prevent its binding to TNFR1 and TNFR2. Preventing the binding of TNF and TNFR2 is important for the generation and function of T reg cells (Tseng W et al. Proceedings of the National Academy of Sciences, 2019 116 (43) 21666-21672). The purpose of this experiment was to investigate whether EN-1206-Fc-MV (monovalent) and EN-1206-Fc-BV (bivalent) and their suppression of TNFR1, is superior to Etanercept (TNF Blocker) in preserving the human regulatory T cell population in cultures of human T cells. This example demonstrates that the number of Tregs (CD4⁺CD25⁺FOXP3⁺) and the level of FOXP3 are higher in human immune cells treated with EN-1206-Fc-BV and EN-1206-Fc-MV compared to same dose of the anti-TNF agent Etanercept (sold as Enbrel®).

### Materials and Methods

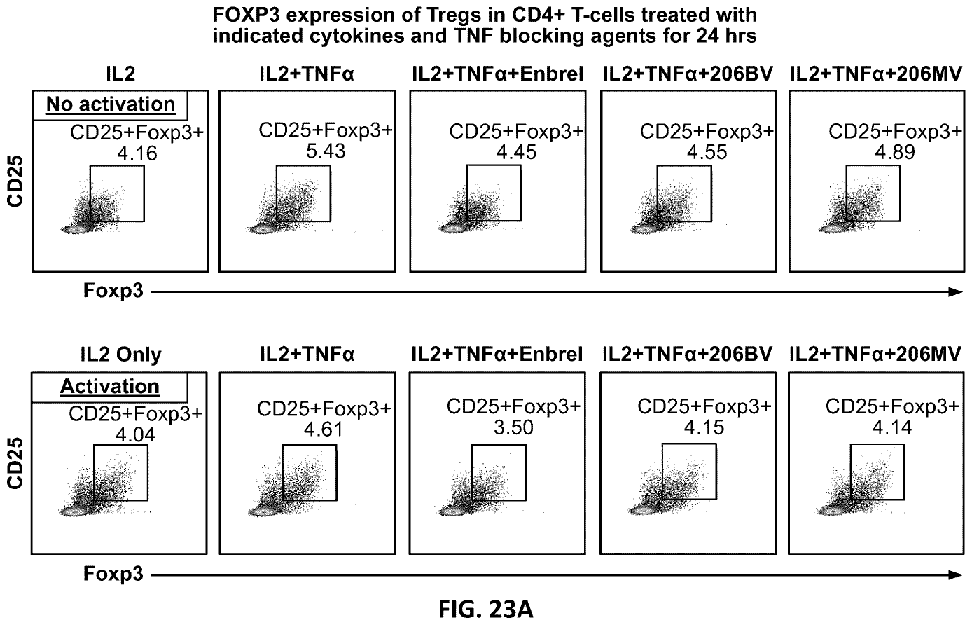

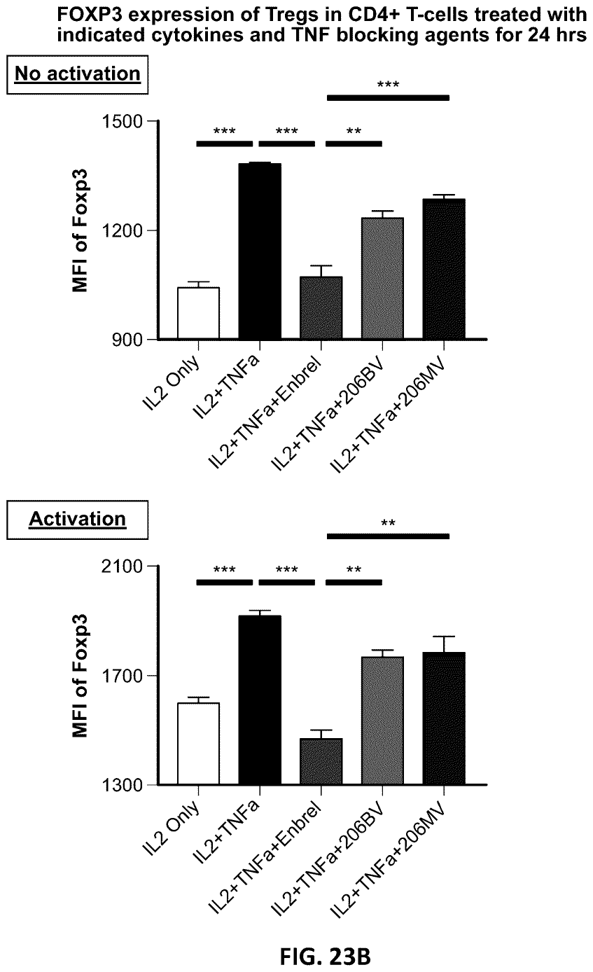

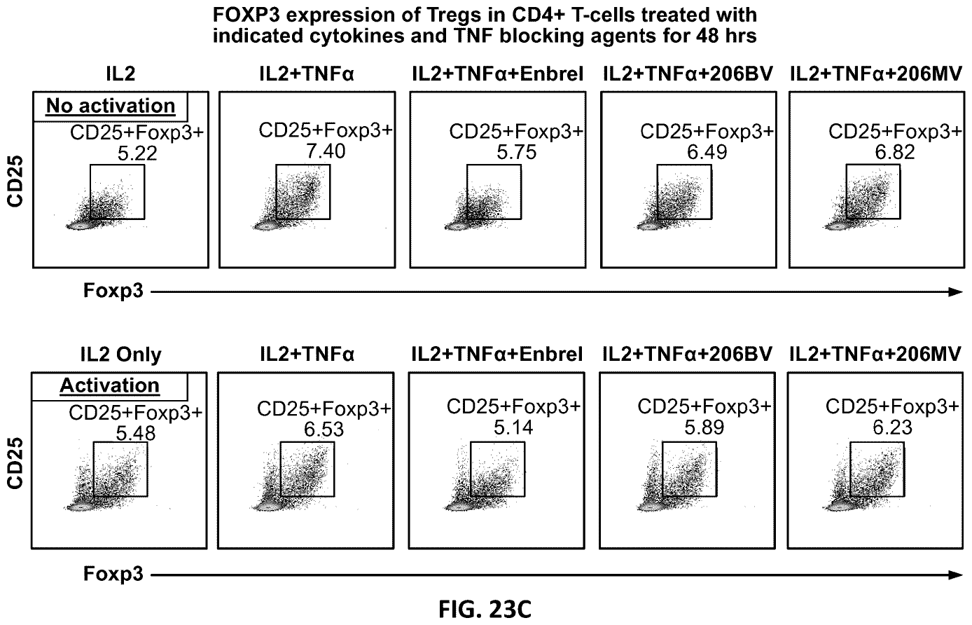

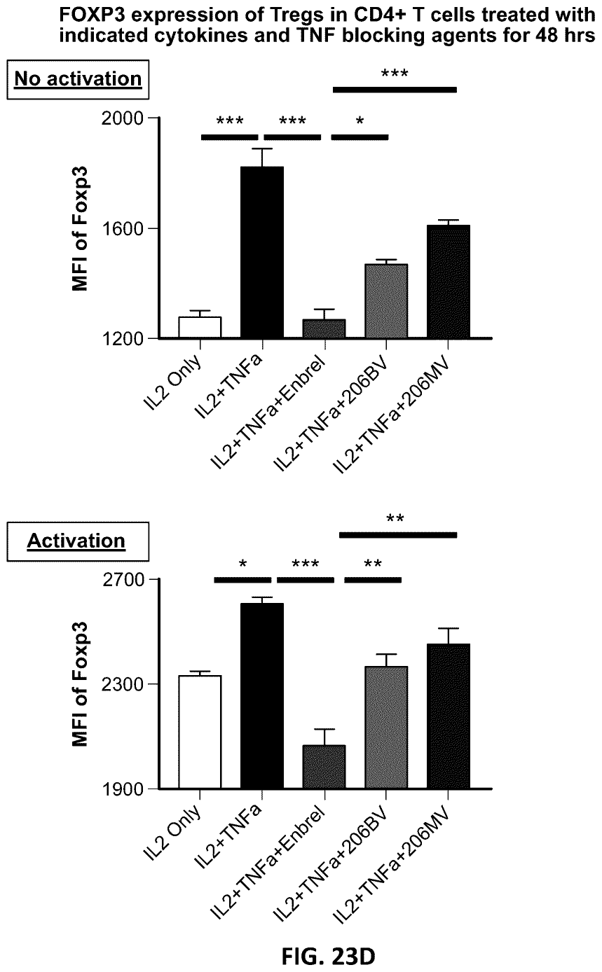

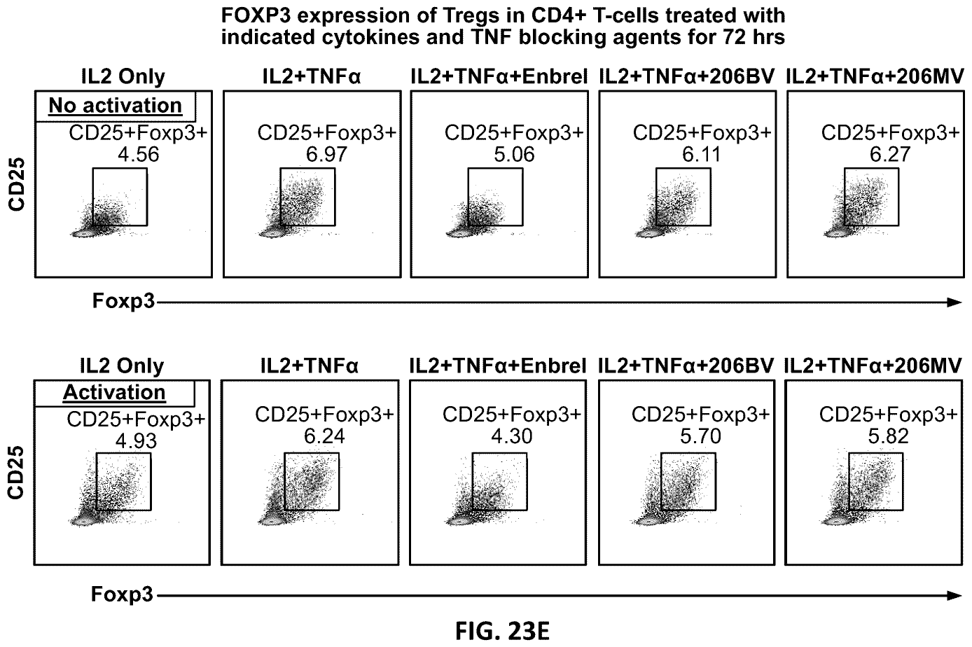

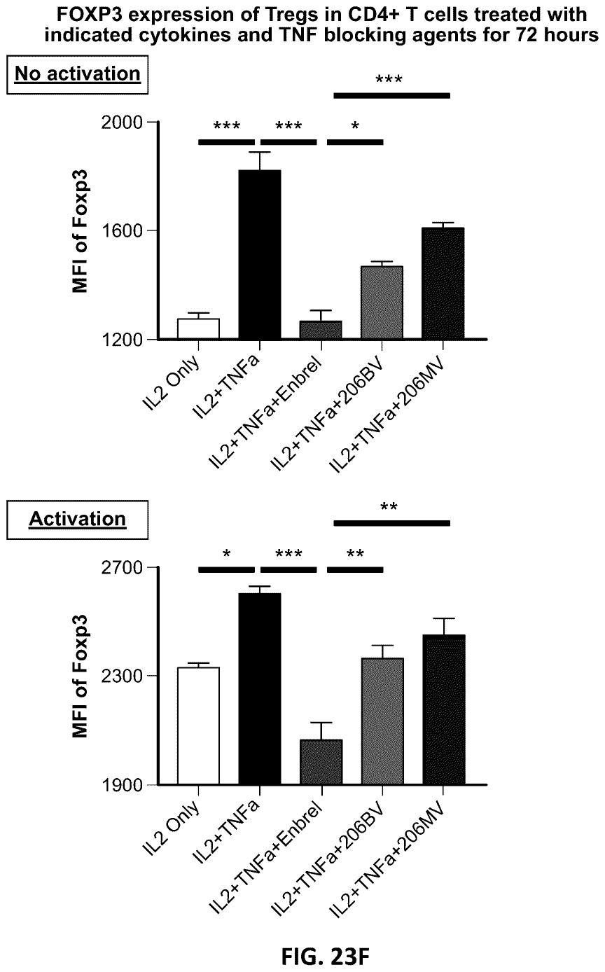

Frozen-preserved human T cells were thawed and cultured. CD4⁺ T cells were thawed and maintained in the presence of IL2 (10 ng/ml) for 2 days. In separate cultures, 2×10⁵ CD4⁺ T cells were treated with TNF-α (100 ng/ml) alone or combined with 10 nM of TNF-α Blocker etanercept (Enbrel®) or 10 nM of either TNFR1 inhibitor EN-1206-Fc-BV (bivalent) or EN-1206-Fc-MV (monovalent) in the absence or presence of 1 g/ml anti-CD3 and anti-CD28 monoclonal antibodies (activating reagents to amplify proliferation of T cells) for 24, 48, and 72 hrs. Results, shown in FIGS. 23A-F were obtained (CD25⁺FOXP3⁺ T cells) using FACS analysis (n=3, mean±SEM). FIGS. 23A-23F show the scatter plots and bar graphs generated from a FACS analysis that demonstrate that EN-1206-Fc-MV and EN-1206-Fc-BV are superior to etanercept (Enbrel®; TNF Blocker) in preserving the human regulatory T cell population in cultures of human T cells (CD25⁺FOXP3⁺ T-Cells, n=3, mean SEM). For the bar-graph representation of the FACS data statistical significance is represented as: *<p.05. **<p.01, ***<p.001 for differences between the samples. The solid bar under each ‘*’ indicates the samples being compared.

### Results and Discussion

Cultures were analyzed by FACS gated for CD25 hi FOXP3⁺(FIG. 23A, 23C, and 23E) without activation (top panels) or with activation (bottom panels). Background levels of staining intensity were lower in the non-activated samples, but in each sample the highest intensity of staining for Treg cells was present in samples treated with TNF+IL2 as shown on the FACS diagram. Median fluorescence intensity for each treatment group at 24, 48, and 72 hrs is depicted in FIGS. 23B, 23D, and 23F. In all instances, human immune cells treated with EN-1206-Fc-BV and EN-1206-Fc-MV resulted in higher levels of Treg cells compared same dose of the anti-TNF agent, etanercept (Enbrel®). These data demonstrate that preferential targeting of TNFR1 (and preservation of TNFR2 function) versus TNF Trapping with etanercept (Enbrel® antibody) sustains the majority of Tregs and results in higher levels of FOXP3, which is a surrogate marker of anti-inflammatory Treg function. This results from higher levels of free TNF as well as lymphotoxin (LT or TNF-0) in the medium, since etanercept (Enbrel®) depletes TNF and LT. Circulating TNF/LT are required for optimal maintenance of Tregs and high levels of FOXP3.

### Example 10

Identification of TNFR1 Binding dAbs

### Summary

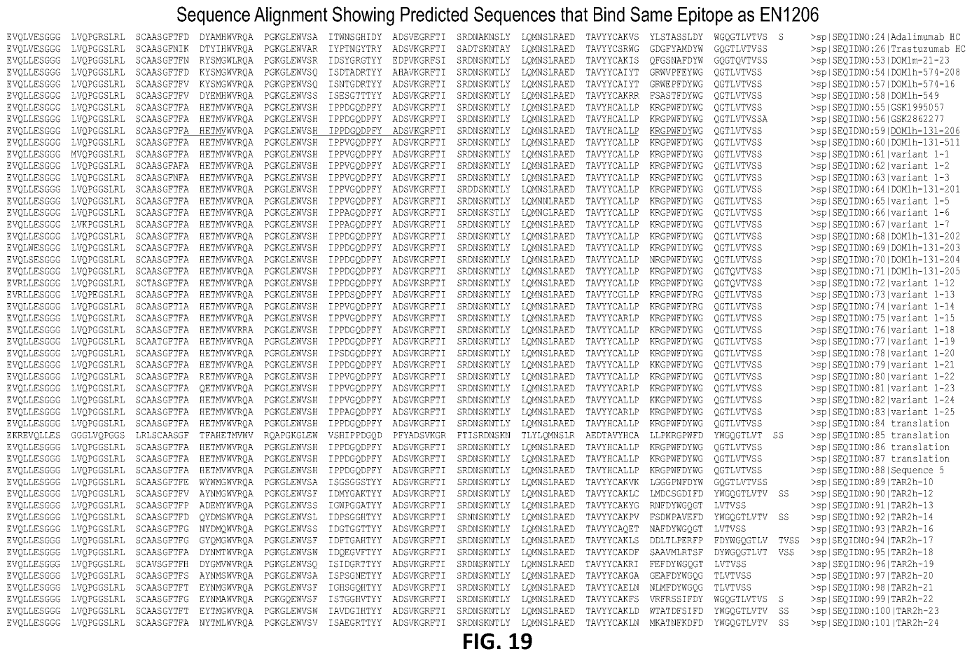

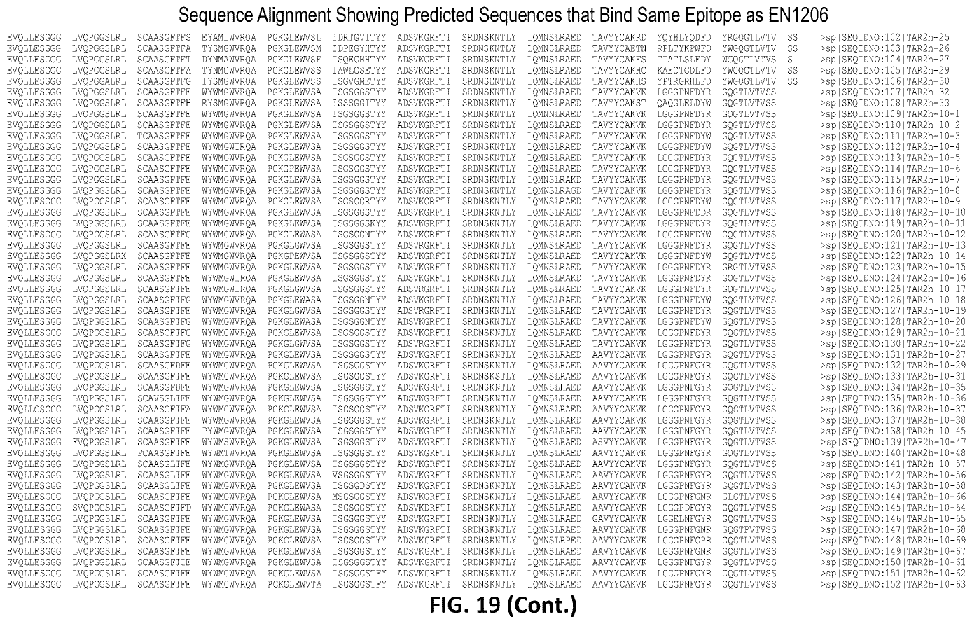

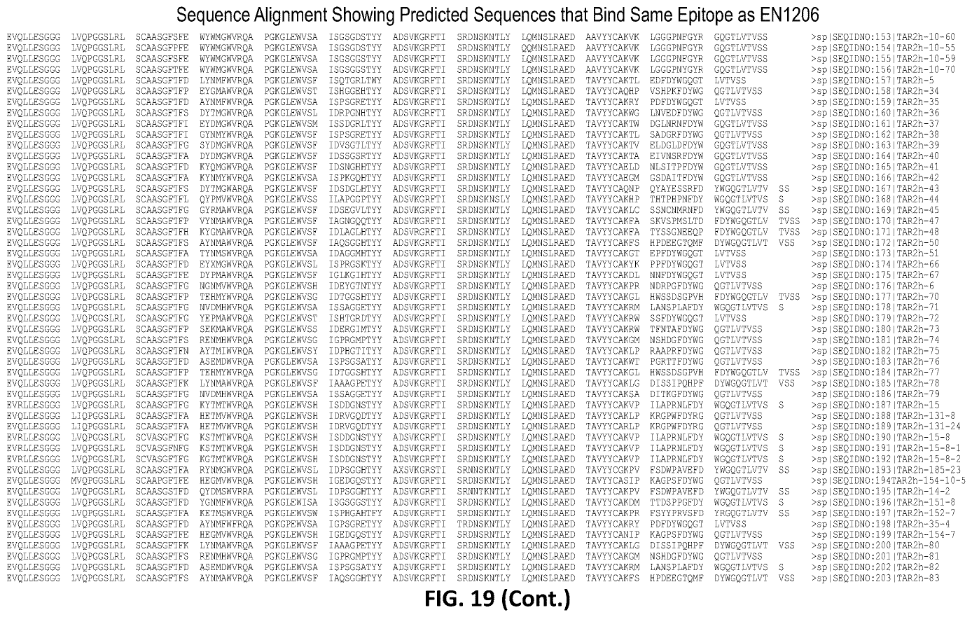

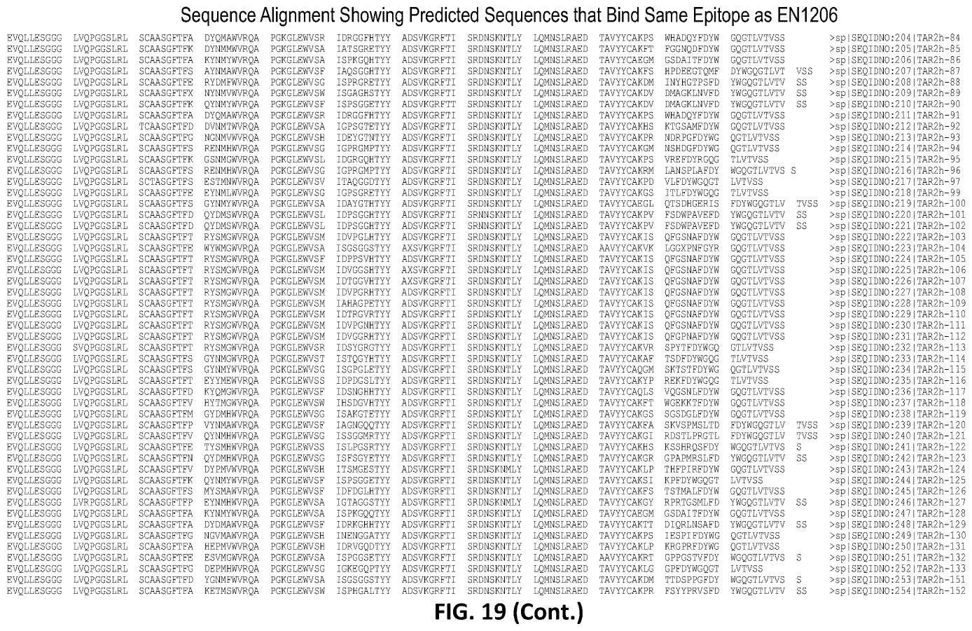

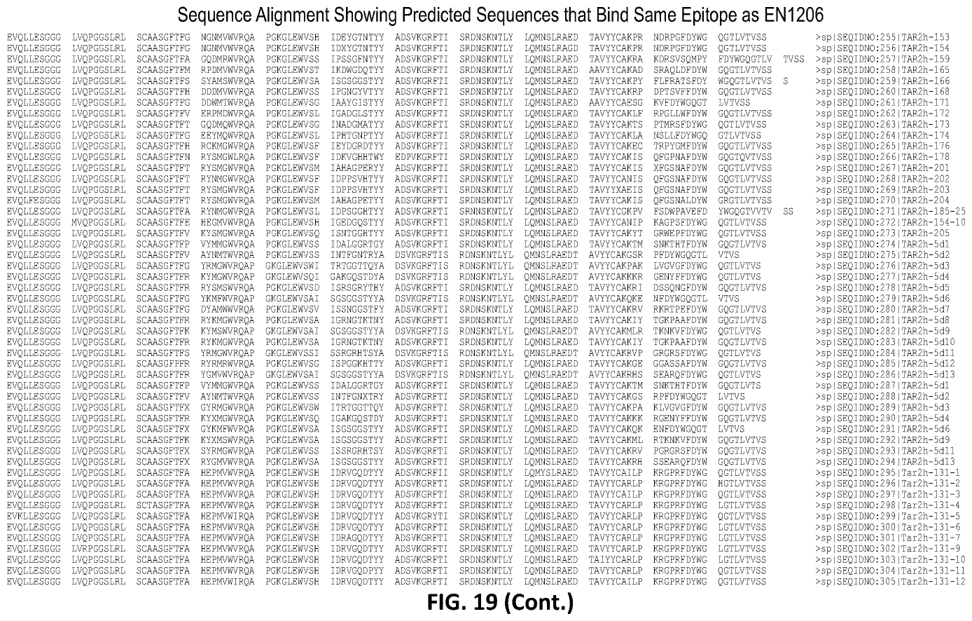

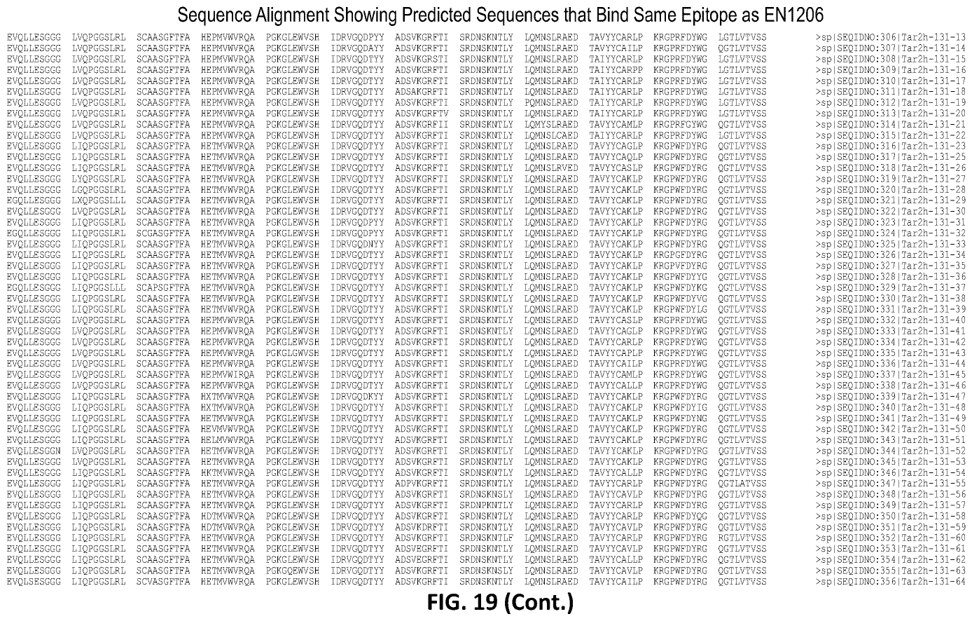

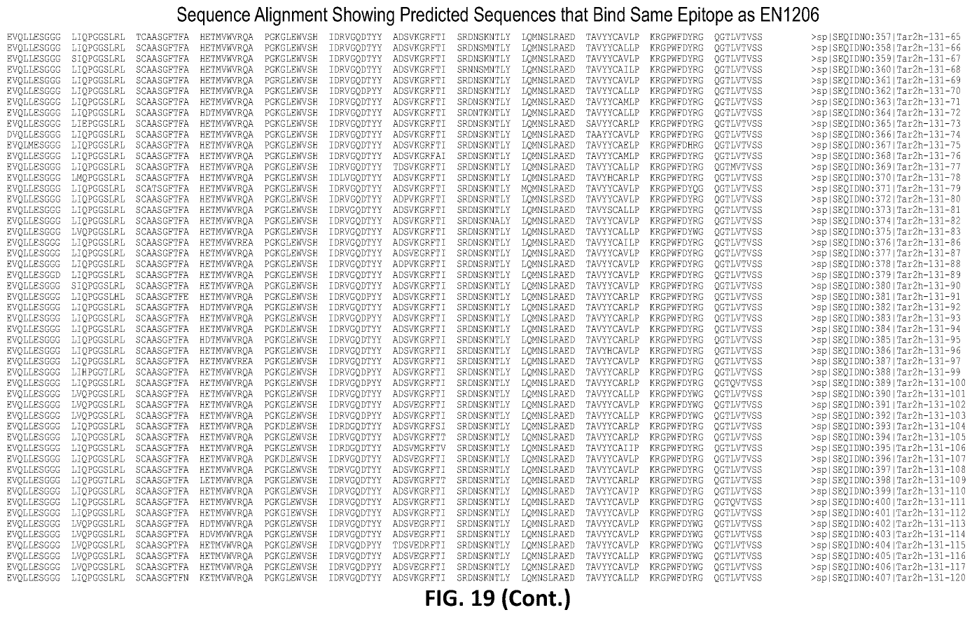

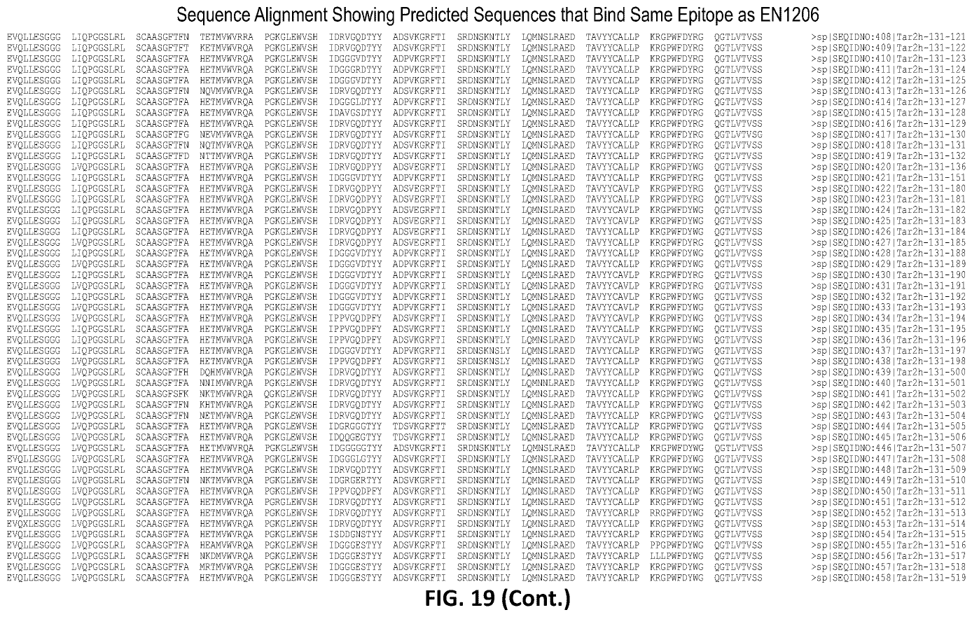

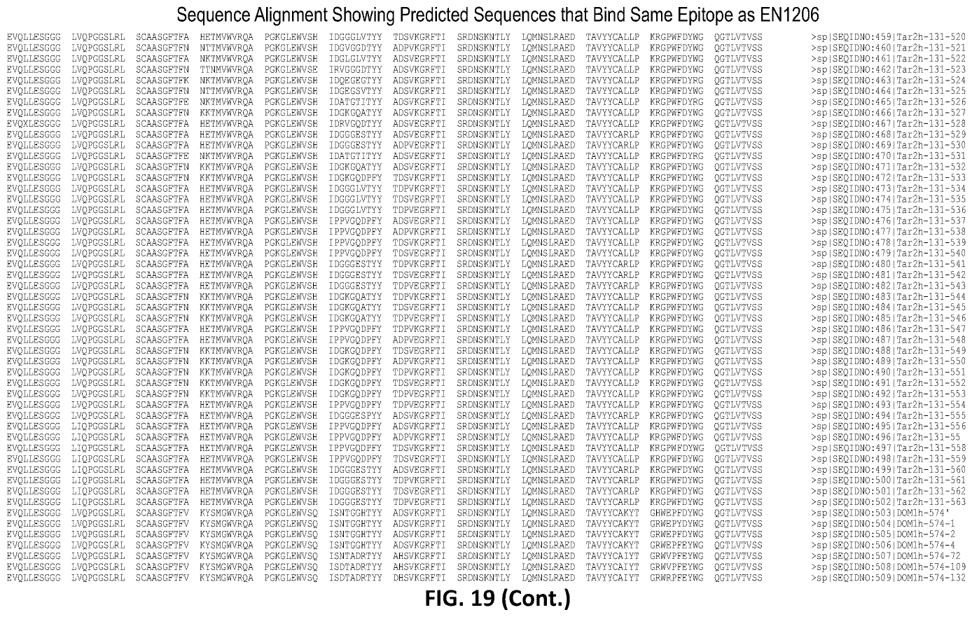

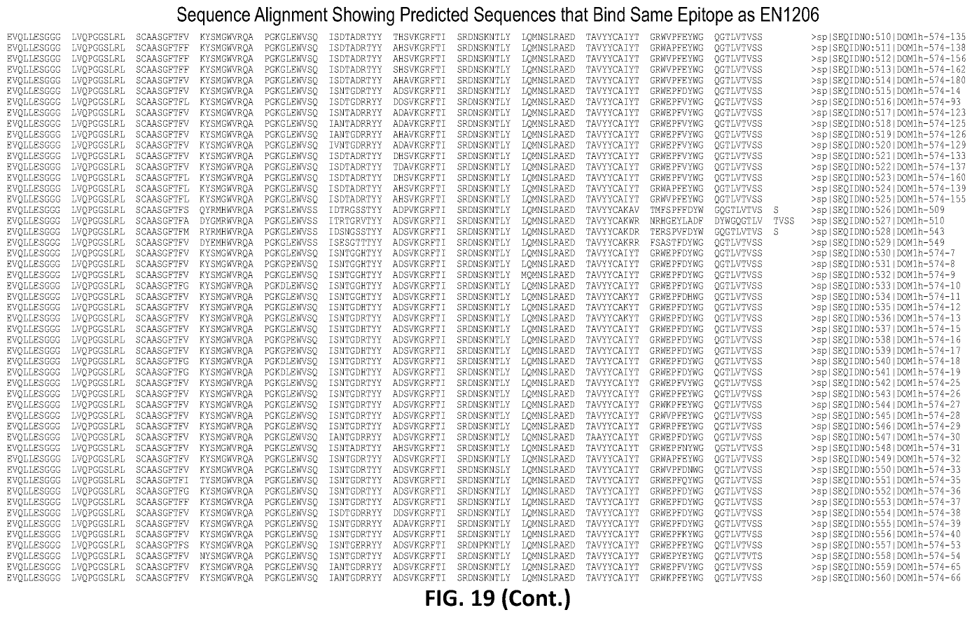

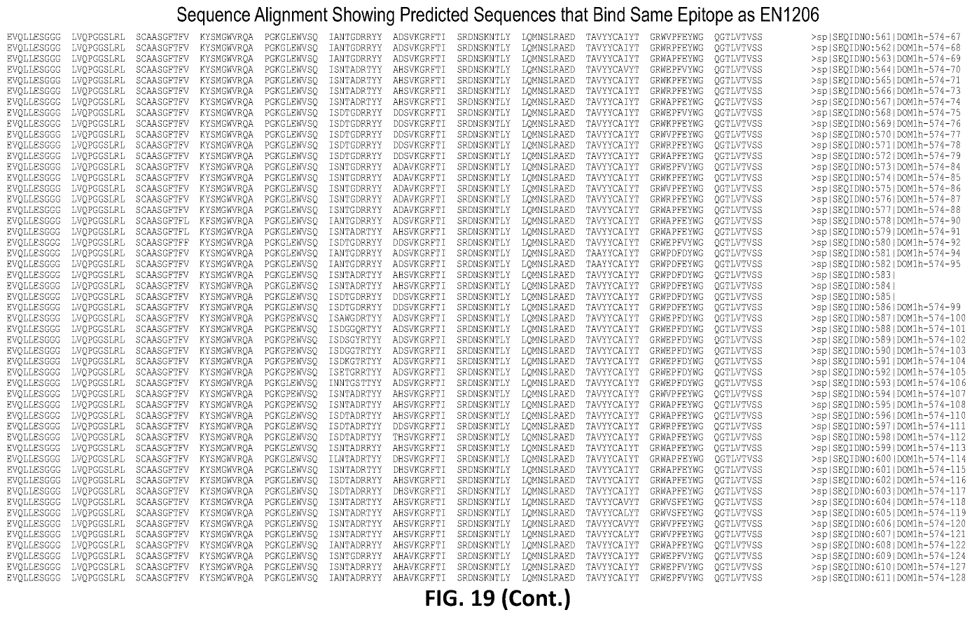

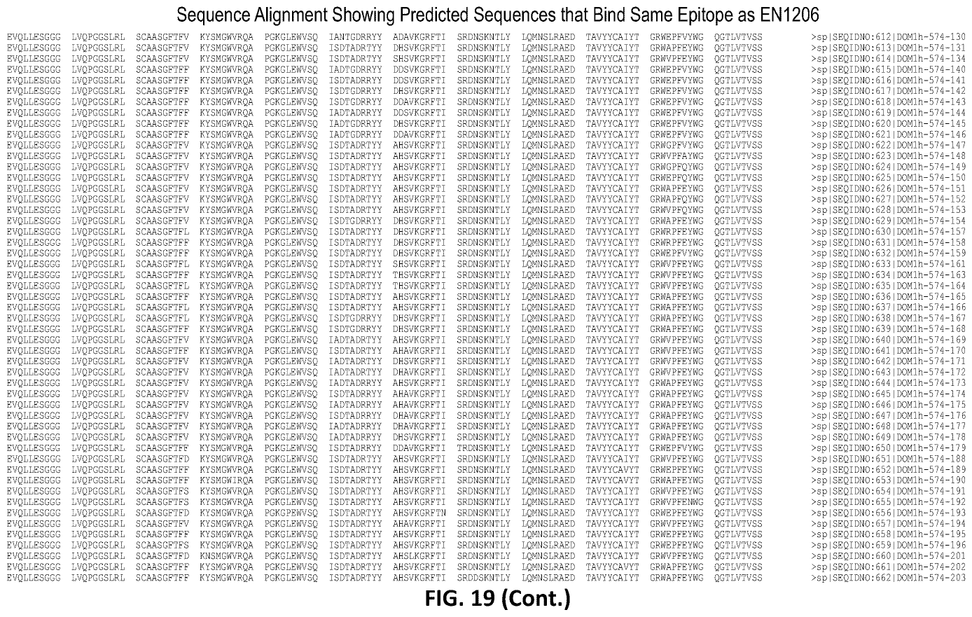

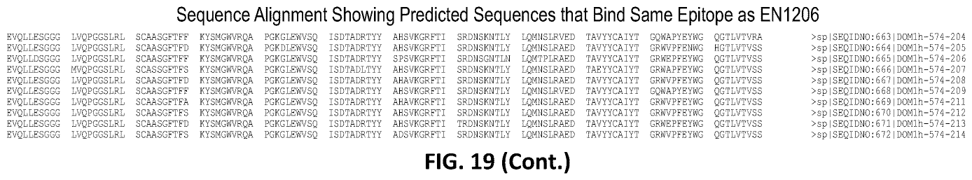

DOM1 h-131-511 (SEQ ID NO: 60) is a V_{H} dAb which binds domain three of human TNFR1 extracellular domain (ECD); EN1206 (DOM1 h-131-206) (SEQ ID NO: 59) is a variant of DOM1 h-131-511 optimized for protease resistance. A multiple sequence alignment (FIG. 19) was performed with each of the polypeptides of SEQ ID Nos: 54-672 to identify which among them bind to the same epitope on TNFR1 as the dAb of SEQ ID NO:59.

### Results and Discussion

EN1206 (DOM1 h-131-206; SEQ ID NO: 59) binds to the domain 3 of TNFR1 ECD with an affinity of 407 pM measured by SPR. EN1206 (DOM1 h-131-206) is an affinity and stability optimized clone derived from the parental clone DOM1 h-131-511. By contrast, EN1208 (DOM1 h-574-208; SEQ ID NO: 54) binds to the domain 4 of TNFR1 ECD with an affinity of affinity is 270 pM measured by SPR. EN1206 and EN1208 bind to separate, non-overlapping epitopes on TNFR1. While EN1208 shows TNFR1 agonist activity.

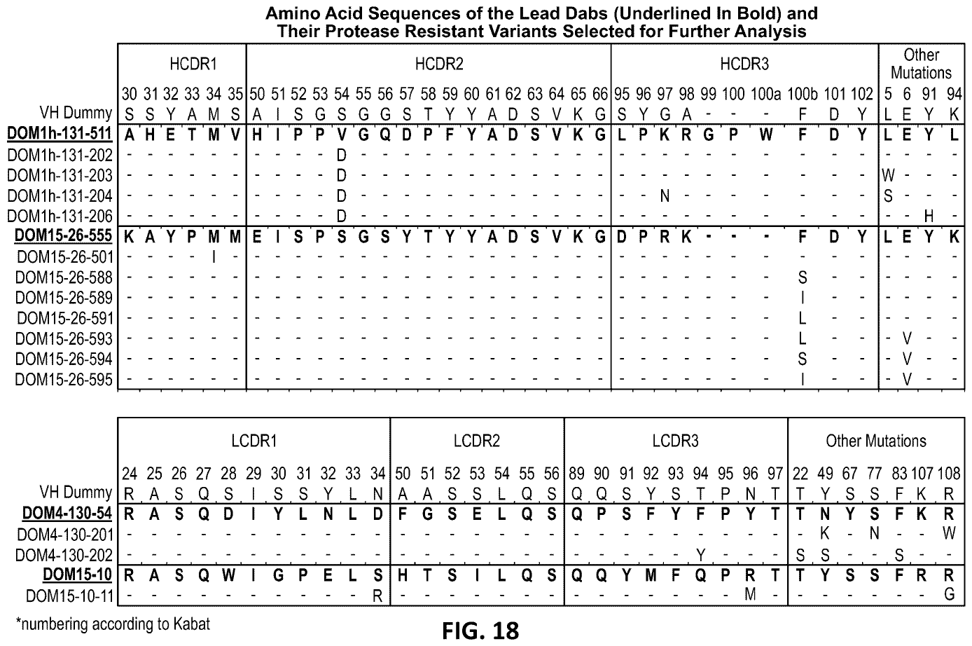

Thirty-six (36) dAbs were identified based on the combined amino acid similarity in the three CDRs (HCDR1, HCDR2, and HCDR3; see FIG. 18) of each sequence to that of DOM1h-131-511 and EN1206 (DOM1 h-131-206). The amino acid sequences of the human CDRs binding domain 3 of TNFR1 ECD are as follows:

HCDR1:
(SEQ ID NO: 1510)
AHETMV
HCDR2:
(SEQ ID NO: 1513)
HIPPDGQDPFYADSVKG
HCDR3:
(SEQ ID NO: 1512)
LPKRGPWFDY

Any sequences having the CDR identity equal or higher than 95% were identified as binding to the same epitope. The DOM1 h-131-511 (parental)-derived variants can bind to the same epitope with very different affinity, but will not bind to a different epitope if their CDRs are highly identical (>90%). The identified dAbs are those of SEQ ID NOs: 55-84, 86-88, 450, 495-498. Constructs containing each of these dAbs in place of the 206 dAb of SEQ ID NO:59 have TNFR1 antagonistic activity, and lack agonist activity.

Since modifications will be apparent to those of skill in the art, it is intended that this invention be limited only by the scope of the appended claims.

What is claimed:

1. A construct that is a tumor necrosis factor receptor 1 (TNFR1) antagonist, comprising two polypeptide chains that are the same or different; wherein:
each chain has the formula 1: 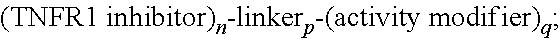
each of n and q is an integer, where n is 0 or 1, and q is 1;
p is 0, 1, 2 or 3;
at least one chain comprises a TNFR1 inhibitor molecule;
the TNFR1 inhibitor molecule binds TNFR1 to inhibit (antagonize) TNFR1;
a linker increases flexibility of the construct, and/or moderates or reduces steric effects of the construct or its interaction with a receptor, and/or increases solubility of the construct in aqueous medium;
the TNFR1 inhibitor comprises a domain antibody (dAb);
the dAb comprises a sequence set forth in any of SEQ ID NOs: 55-84, 86-88, 450, 495-498, which each bind to the same epitope on TNFR1 as the dAb of SEQ ID NO:59; and
an activity modifier is a moiety that modulates or alters the activity or the pharmacological property of the construct compared to the construct in the absence of the activity modifier;
the activity modifier is a modified Fc;
the modified Fc comprises one or more of a)-d):
a) one or more amino acid mutations to increase serum half-life, and, when the construct has one TNFR1 inhibitor and q=2, the Fc has modification(s) to introduce knobs-into-holes;
b) a modification(s) to increase or enhance neonatal Fc receptor (FcRn) recycling;
c) a modification(s) to reduce or eliminate immune effector functions, selected from among one or more of complement-dependent cytotoxicity (CDC), antibody-dependent cell-mediated cytotoxicity (ADCC) and antibody-dependent cell-mediated phagocytosis (ADCP).

2. The construct of claim 1, wherein the dAb binds to the domain 3 of the TNFR1 extracellular domain (ECD) with an affinity of about 300-500 pM as measured by SPR.

3. The construct of claim 1, wherein n is 1 on each chain, whereby the construct is bivalent; or
for one chain, n is 0 and the modified Fc comprises a knob or hole, and for the other chain n is 1, and the Fc comprises a knob or hole whereby construct forms two chains with a single dAb.

4. The construct of claim 1, wherein the modified Fc comprises the mutations designated YTE (M252Y,S254T,T256E with reference to SEQ ID NO:1503) to extend half-life, and the mutations designed LALAPG (L234A,L235A,P329G with reference to residue positions in SEQ ID NO:1503) to minimize its in vivo Fc effector function.

5. The construct of claim 3 that is designated EN-1206 bivalent or EN-1206, wherein the constructs comprise the dAb of SEQ ID NO:59 and do not have TNFR1 agonist activity.

6. The construct of claim 1, that comprises two chains, wherein for one chain n is 1, and for the other chain n is 0, whereby the resulting construct comprises one chain that contains the dAb and modified Fc, and the other chain is a modified Fc, whereby the resulting construct is monovalent.

7. The construct of claim 1, wherein n is 1 for each chain, whereby the construct is bivalent; and the resulting the construct does not have TNRF1 agonist activity.

8. A polypeptide chain for forming a construct of claim 1, wherein a chain is selected from:
a) SEQ ID NO:1488, which is an Fc only comprising, by EU numbering, T366S, L368A,Y407V to form a hole, M252Y, S254T, T256E for increased half-life, L234A, L235A, P329G for minimizing, eliminating, or reducing Fc effector function, and includes a leader sequence, MGWSCIILFLVATATGVHS (SEQ ID NO:1509), that is cleaved off in a cell in which the polypeptide is expressed and comprises the sequence:
MGWSCIILFLVATATGVHSDKTHTCPPCPAPEAAGGPSVFLFPPK
PKDTLYITREPEVTCVVVDVSHEDPEVKFNWYVDGVEVHNAKTKP
REEQYNSTYRVVSVLTVLHQDWLNGKEYKCKVSNKALGAPIEKTI
SKAKGQPREPQVYTLPPSRDELTKNQVSLSCAVKGFYPSDIAVEW
ESNGQPENNYKTTPPVLDSDGSFFLVSKLTVDKSRWQQGNVFSCS
VMHEALHNHYTQKSLSLSPGK;
b) SEQ ID NO:1489, which comprises a dAb and Fc, where the Fc comprises the modifications, by EU numbering, T366W to form a knob, M252Y, S254T, T256E for increased half-life, and L234A, L235A, P329G for minimizing, reducing, or eliminating Fc effector function, and the chain includes a leader sequence, MGWSCIILFLVATATGVHS (SEQ ID NO:1509) that is cleaved off in a cell in which is the polypeptide is expressed:
MGWSCIILFLVATATGVHSEVQLLESGGGLVQPGGSLRLSCAASG
FTFAHETMVWVRQAPGKGLEWVSHIPPDGQDPFYADSVKGRFTIS
RDNSKNTLYLQMNSLRAEDTAVYHCALLPKRGPWFDYWGQGTLVT
VSSDKTHTCPPCPAPEAAGGPSVFLFPPKPKDTLYITREPEVTCV
VVDVSHEDPEVKFNWYVDGVEVHNAKTKPREEQYNSTYRVVSVLT
VLHQDWLNGKEYKCKVSNKALGAPIEKTISKAKGQPREPQVYTLP
PSRDELTKNQVSLWCLVKGFYPSDIAVEWESNGQPENNYKTTPPV
LDSDGSFFLYSKLTVDKSRWQQGNVFSCSVMHEALHNHYTQKSLS
LSPGK;
c) SEQ ID NO:1505, which is an Fc with the modifications, by EU numbering, T366S, L368A, Y407V to form a hole, M252Y, S254T, T256E for increased half-life, L234A, L235A, P329G for minimizing, reducing, or eliminating Fc effector function, and comprises the sequence:
DKTHTCPPCPAPEAAGGPSVFLFPPKPKDTLYITREPEVTCVVVD
VSHEDPEVKFNWYVDGVEVHNAKTKPREEQYNSTYRVVSVLTVLH
QDWLNGKEYKCKVSNKALGAPIEKTISKAKGQPREPQVYTLPPSR
DELTKNQVSLSCAVKGFYPSDIAVEWESNGQPENNYKTTPPVLDS
DGSFFLVSKLTVDKSRWQQGNVFSCSVMHEALHNHYTQKSLSLSP
GK.
and
d) SEQ ID NO:1506, which comprises a dAb and Fc, where the Fc comprises the modifications, by EU numbering, T366W to form a knob, M252Y, S254T, T256E for increased half-life, and L234A, L235A, P329G for minimizing, reducing, or eliminating Fc effector function, and comprises the sequence:
EVQLLESGGGLVQPGGSLRLSCAASGFTFAHETMVWVRQAPGKGL
EWVSHIPPDGQDPFYADSVKGRFTISRDNSKNTLYLQMNSLRAED
TAVYHCALLPKRGPWFDYWGQGTLVTVSSDKTHTCPPCPAPEAAG
GPSVFLFPPKPKDTLYITREPEVTCVVVDVSHEDPEVKFNWYVDG
VEVHNAKTKPREEQYNSTYRVVSVLTVLHQDWLNGKEYKCKVSNK
ALGAPIEKTISKAKGQPREPQVYTLPPSRDELTKNQVSLWCLVKG
FYPSDIAVEWESNGQPENNYKTTPPVLDSDGSFFLYSKLTVDKSR
WQQGNVFSCSVMHEALHNHYTQKSLSLSPGK.

9. A chain for forming the construct of claim 1 that is a monovalent construct comprising two chains, where one chain comprises EN1206 and the Fc with hole, and the other chain comprises EN1206 and the Fc with knob, wherein:
each chain, when expressed in a cell, comprises the leader sequence MGWSCIILFLVATATGVHS that is cleaved when expressed in a cell and is not present in the resulting construct; and
each chain, when expressed in a cell, comprises the leader sequence and comprises the sequences:
a) the first chain is an Fc with modified residues, by EU numbering, T366S, L368A,Y407V to form a hole, M252Y, S254T, T256E for increasing half-life and L234A, L235A, P329G for minimizing, reducing, or eliminating Fc effector function and having the sequence (SEQ ID NO:1488):
MGWSCIILFLVATATGVHSDKTHTCPPCPAPEAAGGPSVFLFPPK
PKDTLYITREPEVTCVVVDVSHEDPEVKFNWYVDGVEVHNAKTKP
REEQYNSTYRVVSVLTVLHQDWLNGKEYKCKVSNKALGAPIEKTI
SKAKGQPREPQVYTLPPSRDELTKNQVSLSCAVKGFYPSDIAVEW
ESNGQPENNYKTTPPVLDSDGSFFLVSKLTVDKSRWQQGNVFSCS
VMHEALHNHYTQKSLSLSPGK,
wherein MGWSCIILFLVATATGVHS (SEQ ID NO:1509) is a leader sequence present when the chain is expressed, but is not present in the two chain monovalent construct; and
b) the second chain comprises the dAb and a modified Fc with T366W to form a knob, M252Y, S254T, T256E for increasing half-life, and L234A, L235A, P329G minimizing, reducing, or eliminating Fc effector function and having the sequence (SEQ ID NO:1489):
MGWSCIILFLVATATGVHSEVQLLESGGGLVQPGGSLRLSCAASGF
TFAHETMVWVRQAPGKGLEWVSHIPPDGQDPFYADSVKGRFTISRD
NSKNTLYLQMNSLRAEDTAVYHCALLPKRGPWFDYWGQGTLVTVSS
DKTHTCPPCPAPEAAGGPSVFLFPPKPKDTLYITREPEVTCVVVDV
SHEDPEVKFNWYVDGVEVHNAKTKPREEQYNSTYRVVSVLTVLHQD
WLNGKEYKCKVSNKALGAPIEKTISKAKGQPREPQVYTLPPSRDEL
TKNQVSLWCLVKGFYPSDIAVEWESNGQPENNYKTTPPVLDSDGSF
FLYSKLTVDKSRWQQGNVFSCSVMHEALHNHYTQKSLSLSPGK,
wherein MGWSCIILFLVATATGVHS (SEQ ID NO:1509) is a leader sequence present when the chain is expressed, but is not present in the two chain construct.

10. The construct of claim 1, comprising two chains, wherein:
a) the construct is a monovalent construct designated EN1206-MV, wherein:
i) chain 1 in the construct comprises the polypeptide of SEQ ID NO:1505 that is a modified Fc with modifications numbered by reference to SEQ ID NO:1503 by EU numbering: T366S,L368A,Y407V, to form a hole; YTE (M252Y,S254T,T256E), for increased half-life, and LALAPG (L234A,L235A,P329G), for minimized in vivo Fc effector function; wherein the resulting chain comprises the sequence:
DKTHTCPPCPAPEAAGGPSVFLFPPKPKDTLYITREPEVTCVVVDV
SHEDPEVKFNWYVDGVEVHNAKTKPREEQYNSTYRVVSVLTVLHQD
WLNGKEYKCKVSNKALGAPIEKTISKAKGQPREPQVYTLPPSRDEL
TKNQVSLSCAVKGFYPSDIAVEWESNGQPENNYKTTPPVLDSDGSF
FLVSKLTVDKSRWQQGNVFSCSVMHEALHNHYTQKSLSLSPGK;
and
ii) chain 2 comprises a monovalent dAb with an Fe knob (T366W) by EU numbering, and is modified for increased half-life and minimized Fc effector function including, respectively, YTE (M252Y,S254T,T256E, by EU numbering), and LALAPG (L234A,L235A,P329G, by EU numbering), wherein the chain comprises the polypeptide of SEQ ID NO:1506:
EVQLLESGGGLVQPGGSLRLSCAASGFTFAHETMVWVRQAPGKGLE
WVSHIPPDGQDPFYADSVKGRFTISRDNSKNTLYLQMNSLRAEDTA
VYHCALLPKRGPWFDYWGQGTLVTVSSDKTHTCPPCPAPEAAGGPS
VFLFPPKPKDTLYITREPEVTCVVVDVSHEDPEVKFNWYVDGVEVH
NAKTKPREEQYNSTYRVVSVLTVLHQDWLNGKEYKCKVSNKALGAP
IEKTISKAKGQPREPQVYTLPPSRDELTKNQVSLWCLVKGFYPSDI
AVEWESNGQPENNYKTTPPVLDSDGSFFLYSKLTVDKSRWQQGNVF
SCSVMHEALHNHYTQKSLSLSPGK;
b) the construct is a bivalent construct, designated EN1206-BV, comprising identical chains, wherein each chain comprises the dAb and Fc with modifications—YTE (M252Y,S254T,T256E, by EU numbering) LALAPG (L234A,L235A,P329G, by EU numbering), wherein each chain comprising the sequence, (SEQ ID NO:1507):
EVQLLESGGGLVQPGGSLRLSCAASGFTFAHETMVWVRQAPGKGLE
WVSHIPPDGQDPFYADSVKGRFTISRDNSKNTLYLQMNSLRAEDTA
VYHCALLPKRGPWFDYWGQGTLVTVSSDKTHTCPPCPAPEAAGGPS
VFLFPPKPKDTLYITREPEVTCVVVDVSHEDPEVKFNWYVDGVEVH
NAKTKPREEQYNSTYRVVSVLTVLHQDWLNGKEYKCKVSNKALGAP
IEKTISKAKGQPREPQVYTLPPSRDELTKNQVSLTCLVKGFYPSDI
AVEWESNGQPENNYKTTPPVLDSDGSFFLYSKLTVDKSRWQQGNVF
SCSVMHEALHNHYTQKSLSLSPGK.

11. A polypeptide chain for forming a construct of claim 1 selected from:
a) SEQ ID NO:1488, which is an Fc only comprising T366S, L368A,Y407V, by EU numbering, to form a hole, M252Y, S254T, T256E, by EU numbering, for increased half-life, L234A, L235A, P329G, by EU numbering, for minimizing, eliminating, or reducing Fc effector function, and includes a leader sequence, MGWSCIILFLVATATGVHS (SEQ ID NO:1509), that is optional and is cleaved off in a cell in which is the polypeptide is expressed and comprises the sequence:
MGWSCIILFLVATATGVHSDKTHTCPPCPAPEAAGGPSVFLFPPKP
KDTLYITREPEVTCVVVDVSHEDPEVKFNWYVDGVEVHNAKTKPRE
EQYNSTYRVVSVLTVLHQDWLNGKEYKCKVSNKALGAPIEKTISKA
KGQPREPQVYTLPPSRDELTKNQVSLSCAVKGFYPSDIAVEWESNG
QPENNYKTTPPVLDSDGSFFLVSKLTVDKSRWQQGNVFSCSVMHEA
LHNHYTQKSLSLSPGK;
b) SEQ ID NO:1489, which comprises a dAb and Fe, where the Fc comprises the modifications T366W to form a knob, M252Y, S254T, T256E, by EU numbering, for increased half-life, and L234A, L235A, P329G, by EU numbering, for minimizing, reducing, or eliminating Fc effector function, and the chain includes a leader sequence, MGWSCIILFLVATATGVHS (SEQ ID NO:1509) that is optional and is cleaved off in a cell in which is the polypeptide is expressed:
MGWSCIILFLVATATGVHSEVQLLESGGGLVQPGGSLRLSCAASGF
TFAHETMVWVRQAPGKGLEWVSHIPPDGQDPFYADSVKGRFTISRD
NSKNTLYLQMNSLRAEDTAVYHCALLPKRGPWFDYWGQGTLVTVSS
DKTHTCPPCPAPEAAGGPSVFLFPPKPKDTLYITREPEVTCVVVDV
SHEDPEVKFNWYVDGVEVHNAKTKPREEQYNSTYRVVSVLTVLHQD
WLNGKEYKCKVSNKALGAPIEKTISKAKGQPREPQVYTLPPSRDEL
TKNQVSLWCLVKGFYPSDIAVEWESNGQPENNYKTTPPVLDSDGSF
FLYSKLTVDKSRWQQGNVFSCSVMHEALHNHYTQKSLSLSPGK;
c) SEQ ID NO:1505, which is an Fc with T366S, L368A, Y407V, by EU numbering, to form a hole, M252Y, S254T, T256E, by EU numbering, for increased half-life, L234A, L235A, P329G, by EU numbering, for minimizing, reducing, or eliminating Fc effector function, and comprises the sequence:
DKTHTCPPCPAPEAAGGPSVFLFPPKPKDTLYITREPEVTCVVVDV
SHEDPEVKFNWYVDGVEVHNAKTKPREEQYNSTYRVVSVLTVLHQD
WLNGKEYKCKVSNKALGAPIEKTISKAKGQPREPQVYTLPPSRDEL
TKNQVSLSCAVKGFYPSDIAVEWESNGQPENNYKTTPPVLDSDGSF
FLVSKLTVDKSRWQQGNVFSCSVMHEALHNHYTQKSLSLSPGK;
d) SEQ ID NO:1506, which comprises a dAb and Fc, where the Fc comprises, by EU numbering, T366W to form a knob, M252Y, S254T, T256E, by EU numbering, for increased half-life, and L234A, L235A, P329G, by EU numbering, for minimizing, reducing, or eliminating Fc effector function, and comprises the sequence:
EVQLLESGGGLVQPGGSLRLSCAASGFTFAHETMVWVRQAPGKGLE
WVSHIPPDGQDPFYADSVKGRFTISRDNSKNTLYLQMNSLRAEDTA
VYHCALLPKRGPWFDYWGQGTLVTVSSDKTHTCPPCPAPEAAGGPS
VFLFPPKPKDTLYITREPEVTCVVVDVSHEDPEVKFNWYVDGVEVH
NAKTKPREEQYNSTYRVVSVLTVLHQDWLNGKEYKCKVSNKALGAP
IEKTISKAKGQPREPQVYTLPPSRDELTKNQVSLWCLVKGFYPSDI
AVEWESNGQPENNYKTTPPVLDSDGSFFLYSKLTVDKSRWQQGNVF
SCSVMHEALHNHYTQKSLSLSPGK.

12. A chain for forming a construct of claim 1, comprising the sequence:
(SEQ ID NO: 1507)
EVQLLESGGGLVQPGGSLRLSCAASGFTFAHETMVWVRQAPGKGLE
WVSHIPPDGQDPFYADSVKGRFTISRDNSKNTLYLQMNSLRAEDTA
VYHCALLPKRGPWFDYWGQGTLVTVSSDKTHTCPPCPAPEAAGGPS
VFLFPPKPKDTLYITREPEVTCVVVDVSHEDPEVKFNWYVDGVEVH
NAKTKPREEQYNSTYRVVSVLTVLHQDWLNGKEYKCKVSNKALGAP
IEKTISKAKGQPREPQVYTLPPSRDELTKNQVSLTCLVKGFYPSDI
AVEWESNGQPENNYKTTPPVLDSDGSFFLYSKLTVDKSRWQQGNVF
SCSVMHEALHNHYTQKSLSLSPGK.

13. A construct of claim 1, comprising two chains, wherein:
a) one chain comprises the polypeptide of SEQ ID NO:1505; and
b) the second chain comprises the polypeptide of SEQ ID NO:1506, whereby the construct comprises a dAb of SEQ ID NO:59 linked to the Fc comprising a knob, T366W, by EU numbering, and is modified to have increased half-life and reduced or eliminated Fc effector function as in the first chain.

14. A construct of claim 1, comprising a dAb of any of SEQ ID NOs: 55-59, 60-84, 86-88, 450, or 495-498 linked directly or via a linker to a modified Fc that comprises M252Y, S254T, T256E, by EU numbering, for increasing half-life and L234A, L235A, P329G by EU numbering, for minimizing, reducing, or eliminating Fc effector function.

15. A TNFR1 antagonist construct of claim 1, comprising a dAb, an optional linker, and a modified Fc, wherein the dAb is DOM1 h-131-206 SEQ ID NO:59:
EVQLLESGGGLVQPGGSLRLSCAASGFTFAHETMVWVRQAPGKGLE
WVSHIPPDGQDPFYADSVKGRFTISRDNSKNTLYLQMNSLRAEDTA
VYHCALLPKRGPWFDYWGQGTLVTVSS.

16. The construct of claim 1 that comprises a linker, wherein the linker is selected from among a chemical linker, a polypeptide linker, and combinations thereof.

17. The construct of claim 16 that comprises a linker between the dAb and Fc portions, wherein the linker is selected from among:
a) a linker that comprises all or a portion of the hinge sequence of trastuzumab, SCDKTH (corresponding to residues 222-227 of SEQ ID NO:26) or up to the full sequence of the hinge region of trastuzumab, that contains or has the sequence EPKSCDKTHTCPPCP (corresponding to residues 219-233 of SEQ ID NO:26), or at least 5, 6, 7, 8, 9, 10, or 11 contiguous residues thereof, or residues ESKYGPPCPPCP, set forth as residues 212-223 of SEQ ID NO:29, or a sequence having at least 98% or 99% sequence identity thereto that is a linker;
b) a linker that is or comprises a glycine-serine (GS) linker;
c) a GS linker selected from among (GlySer)ₙ, where n=1-10; (GlySer₂); (Gly₄Ser)ₙ, where n=1-10; (Gly₃Ser)ₙ, where n=1-5; (SerGly₄)ₙ, where n=1-5; (GlySerSerGly)ₙ, where n=1-5; GSGGSSGG; GSSSGSGSGSSG; GSSSGSGSGSSGG; GGSSGG; GGSSGGSGGSSSG; GSSSGSGSGGSSSGSGSG; GGSSGGSSGGGSSGGSSG; and GSSSGS;
d) a linker that comprises a GS linker and all or a portion of the hinge sequence of trastuzumab, corresponding to residues EPKSCDKTHTCPPCP, set forth as residues 219-233 of SEQ ID NO:26;
e) a linker that comprises a GS linker and comprises the sequence SCDKTH, corresponding to residues 217-222 of SEQ ID NO:31; and
f) a linker that comprises a GS linker and all or a portion of the hinge sequence of nivolumab, corresponding to residues 212-223 of SEQ ID NO:29.

18. The construct of claim 1, wherein a chain comprises a modified Fc, wherein:
the unmodified Fc is an IgG1 or IgG4 Fc;
the IgG1 Fc is selected from the IgG1 Fc of human IgG1, set forth in SEQ ID NO:10, or the IgG1 Fc of trastuzumab, set forth in SEQ ID NO:27;
the IgG4 Fc is selected from the IgG4 Fc of human IgG4, set forth in SEQ ID NO:16, or the IgG4 Fc of nivolumab, set forth in SEQ ID NO:30; and
optionally, the Fc includes one or more modifications to introduce knobs-into-holes, and/or increase or enhance neonatal Fc receptor (FcRn) recycling, and/or reduce or eliminate immune effector functions.

19. The construct of claim 1, wherein the Fc is selected from among:
a) an Fc that comprises knobs-into-holes modifications, wherein:
the knob mutation is selected from among one or more of S354C, T366Y, T366W, and T394W by EU numbering; and
the hole mutation is selected from among one or more of Y349C, T366S, L368A, F405A, Y407T, Y407A, and Y407V by EU numbering;
b) an Fc that comprises modifications to increase or enhance FcRn recycling that is/are selected from among one or more of:
T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering;
c) an Fe that comprises modifications to immune effector functions that are selected from among one or more of complement-dependent cytotoxicity (CDC), antibody-dependent cell-mediated cytotoxicity (ADCC) and antibody-dependent cell-mediated phagocytosis (ADCP);
d) a construct that comprises modification(s) in the Fc to reduce or eliminate immune effector functions, wherein the Fc and modifications are selected from among one or more of:
in IgG1: L235E, L234A/L235A, L234E/L235F/P331S, L234F/L235E/P331S, L234A/L235A/P329G, L234A/L235A/G237A/P238S/H268A/A330S/P331S, G236R/L328R, G237A, E318A, D265A, E233P, N297A, N297Q, N297D, N297G, N297G/D265A, A330L, D270A, P329A, P331A, K322A, V264A, and F241A, by EU numbering; and
in IgG4: L235E, F234A/L235A, S228P/L235E, and S228P/F234A/L235A, by EU numbering;
e) an Fc that is an IgG Fc that comprises one or more of the following modifications:
i) a modification(s) to introduce knobs-into-holes, wherein:
the knob mutation is selected from among one or more of S354C, T366Y, T366W, and T394W by EU numbering; and
the hole mutation is selected from among one or more of Y349C, T366S, L368A, F405A, Y407T, Y407A, and Y407V by EU numbering;
ii) a modification(s) to increase or enhance neonatal Fc receptor (FcRn) recycling, wherein the modification is selected from among one or more of:
T250Q, T250R, M252F, M252W, M252Y, S254T, T256D, T256E, T256Q, V259I, V308F, E380A, M428L, H433K, N434F, N434A, N434W, N434S, N434Y, Y436H, M252Y/T256Q, M252F/T256D, M252Y/S254T/T256E, H433K/N434F/Y436H, N434F/Y436H, T250Q/M428L, T250R/M428L, M428L/N434S, V259I/V308F, V259I/V308F/M428L, E294del/T307P/N434Y, and T256N/A378V/S383N/N434Y, by EU numbering; and
iii) a modification(s) to increase or enhance immune effector functions, wherein:
the immune effector functions are selected from among one or more of CDC, ADCC and ADCP; and
the modification(s) to increase or enhance immune effector functions is selected from among one or more of:
in IgG1: S239D; 1332E; S239D/I332E; S239D/A330L/I332E; S298A/E333A/K334A; F243L/R292P/Y300L/V305I/P396L; L235V/F243L/R292P/Y300L/P396L; F243L/R292P/Y300L; L234Y/G236W/S298A in the first heavy chain and S239D/A330L/I332E in the second heavy chain; L234Y/L235Q/G236W/S239M/H268D/D270E/S298A in the first heavy chain and D270E/K326D/A330M/K334E in the second heavy chain; A327Q/P329A; D265A/S267A/H268A/D270A/K326A/S337A; T256A/K290A/S298A/E333A/K334A; G236A; G236A/I332E; G236A/S239D/I332E; G236A/S239D/A330L/I332E; introduction of a biantennary glycan at residue N297; introduction of an afucosylated glycan at residue N297; K326W; K326A; E333A; K326A/E333A; K326W/E333 S; K326M/E333 S; K222W/T223W; K222W/T223W/H224W; D221W/K222W; C220D/D221C; C220D/D221C/K222W/T223W; H268F/S324T; S267E; H268F; S324T; S267E/H268F/S324T; G236A/I332E/S267E/H268F/S324T; E345R; and E345R/E430G/S440Y, by EU numbering; and
f) an Fc that is modified to increase binding to the inhibitory Fcγ receptor (FcγR) FcγRIIb; and
g) combinations of a)-f).

20. The construct of claim 1 that comprises the dAb of SEQ ID NO:59 or a variant thereof having at least 95% sequence identity thereto and binding to the same epitope as SEQ ID NO:59 linked directly or indirectly via a linker to the modified Fc.

21. The construct of claim 1, comprising two of polypeptide chains of SEQ ID NOs:1505-1507 linked, whereby they form monovalent or divalent constructs, or comprising variants of the chains of SEQ ID NOs:1505-1507 having at least 95% sequence identity thereto and that bind TNRF1, have anti-TNFR1 antagonist activity, and retain the half-life of constructs.

22. A pharmaceutical composition, comprising a construct of claim 1.

23. A method of treating a disease, disorder, or condition that is treated by inhibiting TNFR1, comprising administering a pharmaceutical composition of claim 22.

24. The method of claim 23, wherein the disease, disorder, or condition is one or more of a chronic inflammatory, autoimmune, neurodegenerative, demyelinating, or respiratory disease or disorder, or a disease, condition or disorder characterized by overexpression of TNF or deregulated TNFR1 signaling in its etiology.

25. The method of claim 23, wherein the disease, disorder, or condition is selected from: rheumatoid arthritis (RA), psoriasis, psoriatic arthritis, juvenile idiopathic arthritis (JIA), spondylarthritis, ankylosing spondylitis, Crohn's disease, ulcerative colitis, inflammatory bowel disease (IBD), uveitis, fibrotic diseases, endometriosis, lupus, multiple sclerosis (MS), congestive heart failure, cardiovascular disease, myocardial infarction (MI), atherosclerosis, metabolic diseases, cytokine release syndrome, septic shock, sepsis, acute respiratory distress syndrome (ARDS), severe acute respiratory syndrome (SARS), SARS-CoV-2, influenza, acute and chronic neurodegenerative diseases, demyelinating diseases and disorders, stroke, Alzheimer's disease, Parkinson's disease, Behget's disease, Dupuytren's disease, Tumor Necrosis Factor Receptor-Associated Periodic Syndrome (TRAPS), pancreatitis, type I diabetes, chronic obstructive pulmonary disease (COPD), chronic bronchitis, emphysema, graft rejection, graft versus host disease (GvHD), lung inflammation, pulmonary diseases and conditions, asthma, cystic fibrosis, idiopathic pulmonary fibrosis, acute fulminant viral or bacterial infections, pneumonia, genetically inherited diseases with TNF/TNFR1 as the causative pathologic mediator, periodic fever syndrome, or cancer.

26. A nucleic acid molecule encoding a polypeptide chain in the construct of claim 1.

27. A vector comprising the nucleic acid molecule of claim 26.

28. A cell, comprising the nucleic acid molecule of claim 26, or a vector comprising the nucleic acid.

29. A method for producing a construct or chain, comprising culturing the cell of claim 28 and isolating the resulting construct or chain.

30. The method of claim 29, wherein the chains in the construct are produced in separate cells and the constructs produced following expression in the cell, or a plurality of chains are produced in the same cell and dimers comprising two chains are isolated.
